(19) United States
(12) Patent Application Publication
(10) Pub. No.: US 2026/0028313 A1
(43) Pub. Date: Jan. 29, 2026

(54) ONIUM SALT, CHEMICALLY AMPLIFIED RESIST COMPOSITION, AND PATTERN FORMING PROCESS

(71) Applicant: Shin-Etsu Chemical Co., Ltd., Tokyo (JP)

(72) Inventor: Masahiro Fukushima, Joetsu-shi (JP)

(73) Assignee: Shin-Etsu Chemical Co., Ltd., Tokyo (JP)

(21) Appl. No.: 19/268,097

(22) Filed: Jul. 14, 2025

(30) Foreign Application Priority Data
Jul. 24, 2024 (JP) 2024-118718

(51) Int. Cl. C07C 381/12; G03F 7/00; G03F 7/029; G03F 7/039

(52) U.S. Cl. CPC C07C 381/12; G03F 7/029; G03F 7/0392; G03F 7/70025; G03F 7/70033; C07C 2602/02

(57) ABSTRACT

Provided are an onium salt, a chemically amplified resist composition comprising the onium salt, and a pattern forming process using the chemically amplified resist composition, the chemically amplified resist composition exhibiting a high sensitivity, high resolution, improved lithography properties including EL, LWR, CDU and DOF, and resist pattern collapse resistance, when processed by photolithography using high-energy radiation independent of whether it is of positive or negative tone. An onium salt has the formula (1):

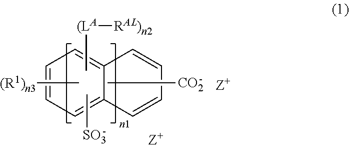

### CROSS-REFERENCE TO RELATED APPLICATION

This non-provisional application claims priority under 35 U.S.C. § 119(a) on Patent Application No. 2024-118718 filed in Japan on Jul. 24, 2024, the entire contents of which are hereby incorporated by reference.

### TECHNICAL FIELD

The present invention relates to an onium salt, a chemically amplified resist composition, and a pattern forming process.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using extreme ultraviolet (EUV) of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To ensure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post exposure bake (PEB) fails, resulting in drastic reductions of sensitivity and contrast.

A triangular tradeoff relationship among sensitivity, resolution, and edge roughness (LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Resist compositions adapted for the ArF lithography are typically based on (meth)acrylate polymers having acid labile groups. These acid labile groups undergo deprotection reaction when an acid generator capable of generating a sulfonic acid which is substituted at α-position with fluorine (referred to as “α-fluorinated sulfonic acid”, hereinafter) is used, but not when an acid generator capable of generating a sulfonic acid which is not substituted at α-position with fluorine (referred to as “α-non-fluorinated sulfonic acid,” hereinafter) or carboxylic acid is used. When a sulfonium or iodonium salt capable of generating α-fluorinated sulfonic acid is mixed with a sulfonium or iodonium salt capable of generating α-non-fluorinated sulfonic acid, the sulfonium or iodonium salt capable of generating α-non-fluorinated sulfonic acid undergoes ion exchange with the α-fluorinated sulfonic acid. Through the ion exchange, the α-fluorinated sulfonic acid once generated upon light exposure is converted back to the sulfonium or iodonium salt. Then the sulfonium or iodonium salt of α-non-fluorinated sulfonic acid or carboxylic acid functions as a quencher. Patent Document 3 discloses a resist composition comprising a sulfonium or iodonium salt capable of generating carboxylic acid as the quencher.

Sulfonium salt type quenchers capable of generating carboxylic acid are known. Proposed thus far are sulfonium salts of salicylic acid and β-hydroxycarboxylic acid (Patent Document 4), salicylic acid derivatives (Patent Documents 5 and 6), fluorosalicylic acids (Patent Document 7), hydroxynaphthoeic acid (Patent Document 8), salicylic acids having an iodized aromatic substituent group introduced therein (Patent Document 9), and salicylic acids having a cyclic acetal structure introduced therein (Patent Document 10). Salicylic acid is quite effective for suppressing acid diffusion due to the intramolecular hydrogen bond of carboxy group and hydroxy group.

It is pointed out that the agglomeration of a quencher causes to degrade the dimensional uniformity or CDU of resist patterns. It is thus expected that the CDU of resist patterns is improved by preventing the quencher from agglomerating in the resist film, for achieving a uniform distribution of the quencher.

In connection with the demand for further miniaturization, there is a problem that upon development of a positive resist film in an alkaline developer, the resist film is swollen with the developer so that pattern collapse may occur upon small-size pattern formation. To solve the problem associated with miniaturization, the development of an effective material for a new resist composition is important. It is desired to have an onium salt type quencher having a high sensitivity, fully controlled acid diffusion, acceptable solvent solubility, and effective prevention of pattern collapse.

Citation List

| | |
|---|---|
| Patent Document 1: | JP-A 2006-45311 |
| Patent Document 2: | JP-A 2006-178317 |
| Patent Document 3: | JP-A 2007-114431 |
| Patent Document 4: | WO 2018/159560 |
| Patent Document 5: | JP-A 2020-203984 |
| Patent Document 6: | JP-A 2020-91404 |
| Patent Document 7: | JP-A 2020-91312 |
| Patent Document 8: | JP-A 2019-120760 |
| Patent Document 9: | JP-A 2022-77505 |
| Patent Document 10: | WO 2023/189502 |
| Non-Patent Document 1: | SPIE Vol. 6520 65203L-1 (2007) |

### SUMMARY OF THE INVENTION

In conjunction with the demand for higher resolution of resist patterns, a prior art resist composition comprising an acid generator of sulfonium salt type and a quencher fails to fully suppress acid diffusion. This raises the problem that lithography properties including contrast, exposure latitude (EL), LWR, CDU, and depth of focus (DOF) are degraded. Another problem arising upon formation of small-size patterns is pattern collapse by swell.

An object of the present invention is to provide an onium salt and a chemically amplified resist composition comprising the onium salt, the chemically amplified resist composition exhibiting a high sensitivity, high resolution, improved lithography properties including EL, LWR, CDU and DOF, and resist pattern collapse resistance, when processed by photolithography using high-energy radiation such as deep UV, EB, or EUV, independent of whether it is of positive or negative tone. Another object is to provide a pattern forming process using the chemically amplified resist composition.

As a result of intensive studies to achieve the above object, the present inventors have found that an onium salt containing an anion having a dianion structure having a carboxylate anion and a sulfonate anion on one aromatic ring and having a hydroxy group or a hydroxy group protected by an acid labile group in a partial structure and an onium cation is excellent in solvent solubility, a chemically amplified resist composition using the onium salt as a quencher has a high sensitivity, high contrast, and improved lithography properties including EL, LWR, CDU, and DOF, and is extremely effective in suppressing pattern collapse during formation of small-size patterns, thereby completing the present invention.

That is, the present invention provides an onium salt, a chemically amplified resist composition, and a pattern forming process.
1. An onium salt having the formula (1):

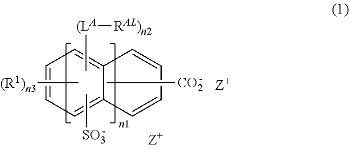

wherein n1 is 0 or 1, n2 is 1, 2, or 3, n3 is 0, 1, or 2, provided that 1≤n2+n3≤4 when n1 is 0, and 1≤n2+n3≤6 when n1 is 1,
   R¹ is a halogen atom, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n3 is 2, two R¹ may be identical or different, and two R¹ may bond together to form a ring with the carbon atoms to which they are attached,
   R^{AL} is a hydrogen atom or an acid labile group,
   L^{A} is an oxygen atom when R^{AL} is a hydrogen atom, and is an ether bond, an ester bond, or a carbonate bond when R^{AL} is an acid labile group, and
   Z⁺ is each independently an onium cation.
2. The onium salt of 1 wherein R^{AL} is an acid labile group having the formula (AL-1) or (AL-2):

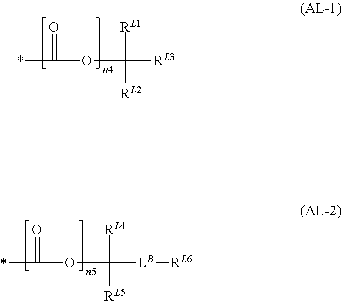

wherein n4 is 0 or 1, n5 is 0 or 1,
   R^{L1}, R^{L2}, and R^{L3} are each independently a C₁-C₁₂ hydrocarbyl group in which some —CH₂— may be replaced by —O— or —S—, when the hydrocarbyl group contains an aromatic ring, some or all of the hydrogen atoms on the aromatic ring may be substituted by a halogen atom, a cyano group, a nitro group, an optionally halogenated C₁-C₄ alkyl group, or an optionally halogenated C₁-C₄ alkoxy group, R^{L1} and R^{L2} may bond together to form a ring with the carbon atom to which they are attached, some —CH₂— in the ring may be replaced by —O— or —S—,
   R^{L4} and R^{L5} are each independently a hydrogen atom or a C₁-C₁₀ hydrocarbyl group, R^{L6} is a C₁-C₂₀ hydrocarbyl group in which some —CH₂— may be replaced by —O— or —S—, R^{L5} and R^{L6} may bond together to form a C₃-C₂₀ heterocyclic group with the carbon atom and L^{B} to which they are attached, some —CH₂— in the heterocyclic group may be replaced by —O— or —S—,
   L^{B} is —O— or —S—, and
   * designates a point of attachment to the adjacent —O—.
3. The onium salt of 1 or 2 having the formula (1A):

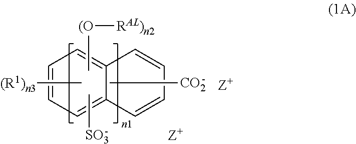

wherein R¹, R^{AL}, n1 to n3, and Z⁺ are as defined above.
4. The onium salt of 3 having the formula (1B):

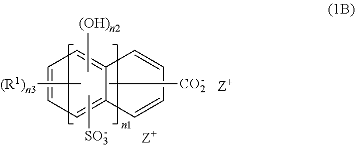

wherein R¹, n1 to n3, and Z⁺ are as defined above.
5. The onium salt of any one of 1 to 4 wherein Z⁺ is a sulfonium cation having the formula (Z-1), an iodonium cation having the formula (Z-2), or an ammonium cation having the formula (Z-3):

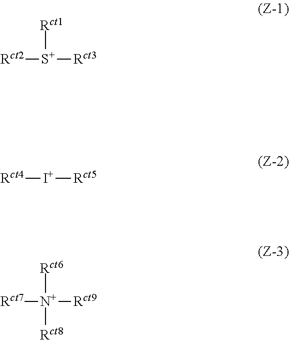

wherein R^{ct1} to R^{ct9} are each independently a halogen atom or a C₁-C₃₀ hydrocarbyl group which may contain a heteroatom, R^{ct1} and R^{ct2} may bond together to form a ring together with the sulfur atom to which they are attached, and any two of R^{ct6} to R^{ct9} may be bond together to form a ring together with the nitrogen atom to which they are attached.
6. The onium salt of any one of 1 to 4 wherein Z⁺ is a sulfonium cation having the formula (Z-4):

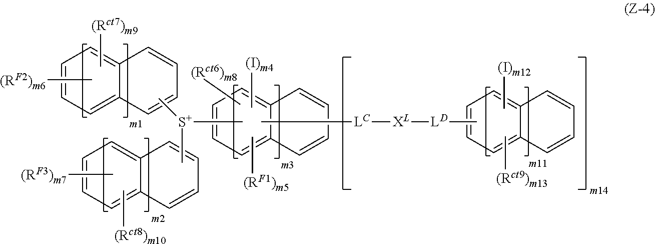

wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, provided that 0≤m6+m9≤4 when m1 is 0 and 0≤m6+m9≤6 when m1 is 1, 0≤m7+m10≤4 when m2 is 0 and 0≤m7+m10≤6 when m2 is 1, 1≤m4+m5+m8+m14≤4 when m3 is 0 and 1≤m4+m5+m8+m14≤6 when m3 is 1, 0≤m12+m13≤4 when m11 is 0 and 0≤m12+m13≤6 when m11 is 1, and m4+m12≥1,
   R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, a plurality of R^{F1} may be identical or different when m5 is 2 or more, a plurality of R^{F2} may be identical or different when m6 is 2 or more, a plurality of R^{F3} may be identical or different when m7 is 2 or more,
   R^{ct6} to R^{ct9} each are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6} may be identical or different and two R^{ct6} may bond together to form a ring with the carbon atoms to which they are attached, when m9 is 2, two R^{ct7} may be identical or different and two R^{ct7} may bond together to form a ring with the carbon atoms to which they are attached, when m10 is 2, two R^{ct8} may be identical or different and two R^{ct8} may bond together to form a ring with the carbon atoms to which they are attached, and when m13 is 2, two R^{ct9} may be identical or different and two R^{ct9} may bond together to form a ring with the carbon atoms to which they are attached,
   the aromatic rings directly bonded to S⁺ in the sulfonium cation may bond together to form a ring with S⁺,
   L^{C} and L^{D} are each independently a single bond, an ether bond, an ester bond, an amide bond, a sulfonate ester bond, a sulfonamide bond, a carbonate bond, or a carbamate bond, and
   X^{L} is a single bond or a C₁-C₄₀ hydrocarbylene group which may contain a heteroatom.
7. The onium salt of 6 wherein the sulfonium cation having formula (Z-4) has the formula (Z-4-1):

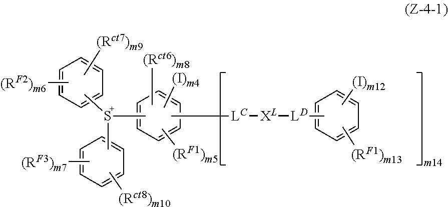

wherein m4 to m10, m12 to m14, R^{F1} to R^{F3}, R^{ct6} to R^{ct9}, L^{C}, L^{D}, and X^{L} are as defined above.
8. The onium salt of 7 wherein the sulfonium cation having formula (Z-4-1) has the formula (Z-4-2):

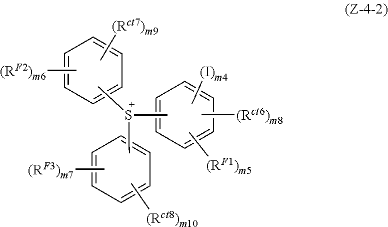

wherein m4 to m10, R^{F1} to R^{F3}, and R^{ct6} to R^{ct8} are as defined above.
9. A quencher comprising the onium salt of any one of 1 to 8.
10. A chemically amplified resist composition comprising the quencher of 9.
11. The chemically amplified resist composition of 10, further comprising a base polymer comprising repeat units of at least one type selected from repeat units having the formula (a1), repeat units having the formula (a2), and repeat units having the formula (a3):

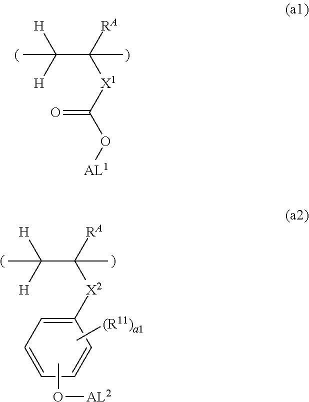

wherein R^{A} is each independently a hydrogen atom, a fluorine atom, a methyl group, or a trifluoromethyl group,
   X¹ is a single bond, a phenylene group, a naphthylene group, *—C(═O)—O—X¹¹—, or *—C(═O)—NH—X¹¹—, the phenylene group or the naphthylene group may be substituted by a hydroxy group, a nitro group, a cyano group, an optionally fluorinated C₁-C₁₀ saturated hydrocarbyl group, an optionally fluorinated C₁-C₁₀ saturated hydrocarbyloxy group, or a halogen atom, X¹¹ is a C₁-C₁₀ saturated hydrocarbylene group, a phenylene group, or a naphthylene group which may contain a hydroxy group, an ether bond, an ester bond, or a lactone ring,
   X² is a single bond, *—C(═O)—O—, or *—C(═O)—NH—,
   * designates a point of attachment to the carbon atom in the backbone,
   R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R¹¹ may be identical or different when a1 is 2, 3, or 4,
   AL¹ and AL² are each independently an acid labile group,
   a1 is 0, 1, 2, 3, or 4,

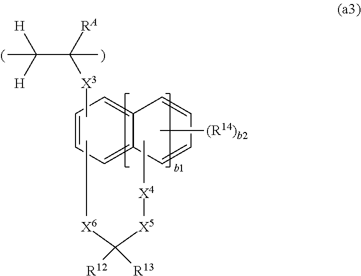

wherein b1 is 0 or 1, b2 is 0, 1, 2, or 3 when b1 is 0, and is 0, 1, 2, 3, 4, or 5 when b1 is 1,
   R^{A} is a hydrogen atom, a fluorine atom, a methyl group, or a trifluoromethyl group,
   X³ is a single bond, *—C(═O)—O—, or *—C(═O)—NH—, * designates a point of attachment to the carbon atom in the backbone,
   X⁴ is a single bond, a C₁-C₄ aliphatic hydrocarbylene group, a carbonyl group, a sulfonyl group, or a group obtained by combining the foregoing,
   X⁵ and X⁶ are each independently an oxygen atom or a sulfur atom, provided that X⁴ and X⁶ are bonded to vicinal carbon atoms on the aromatic ring,
   R¹² and R¹³ are each independently a hydrogen atom or a C₁-C₂₀ hydrocarbyl group which may contain a heteroatom, R¹² and R¹³ may bond together to form a ring with the carbon atom to which they are attached,
   R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, and a plurality of R¹⁴ may be identical or different and the plurality of R¹⁴ may bond together to form a ring with the carbon atoms on the aromatic ring to which they are attached when b2 is 2 or more.
12. The chemically amplified resist composition of 11 wherein the base polymer comprises repeat units of at least one type selected from repeat units having the formula (b1) and repeat units having the formula (b2):

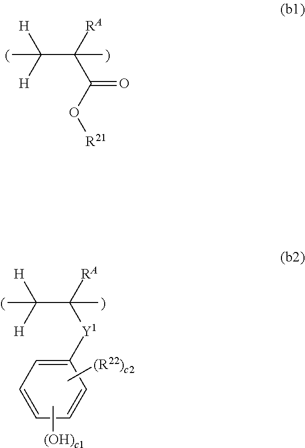

wherein R^{A} is each independently a hydrogen atom, a fluorine atom, a methyl group, or a trifluoromethyl group,
   Y¹ is a single bond or *—C(═O)—O—, * designates a point of attachment to the carbon atom in the backbone,
   R²¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
   R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²² may be identical or different when c2 is 2, 3, or 4,
   c1 is 1, 2, 3, or 4, c2 is 0, 1, 2, 3, or 4, and provided that 1≤c1+c2≤5.
13. The chemically amplified resist composition of 11 or 12 wherein the base polymer comprises repeat units of at least one type selected from repeat units having the formula (c1), repeat units having the formula (c2), repeat units having the formula (c3), repeat units having the formula (c4), and repeat units having the formula (c5):

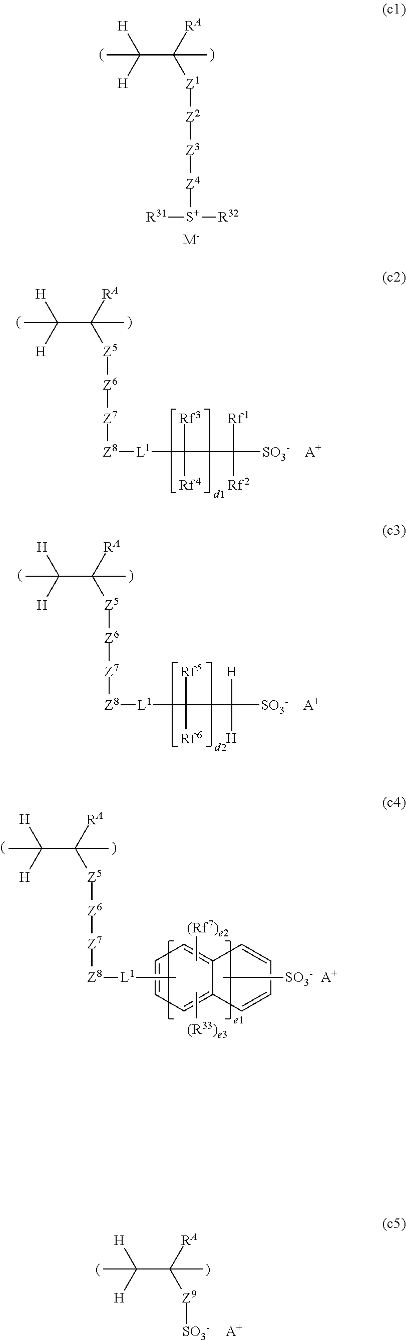

wherein d1 and d2 are each independently 0, 1, 2, or 3, e1 is 0 or 1, e2 is 0, 1, 2, 3, or 4, e3 is 0, 1, 2, 3, or 4, provided that 0≤e2+e3≤4 when e1 is 0 and 0≤e2+e3≤6 when e1 is 1,
   R^{A} is each independently a hydrogen atom, a fluorine atom, a methyl group, or a trifluoromethyl group,
   Z¹ is a single bond or an optionally substituted phenylene group,
   Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining the foregoing, which may contain a halogen atom, a carbonyl group, an ester bond, an ether bond, or a hydroxy group,
   Z³ is a single bond, an ether bond, an ester bond, an amide bond, a sulfonate ester bond, a sulfonamide bond, a carbonate bond, or a carbamate bond,
   Z⁴ is a single bond, or a C₁-C₆ aliphatic hydrocarbylene group, a phenylene group, or a divalent group obtained by combining the foregoing, which may contain a halogen atom, a carbonyl group, an ester bond, an ether bond, or a hydroxy group,
   Z⁵ is each independently a single bond, an optionally substituted phenylene group, a naphthylene group, or *—C(═O)—O—Z⁵¹—, Z⁵¹ is a C₁-C₁₀ aliphatic hydrocarbylene group, a phenylene group, or a naphthylene group which may contain a halogen atom, a hydroxy group, an ether bond, an ester bond, or a lactone ring,
   Z⁶ is a single bond, an ether bond, an ester bond, an amide bond, a sulfonate ester bond, a sulfonamide bond, a carbonate bond, or a carbamate bond,
   Z⁷ is each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
   Z⁸ is each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom,
   Z⁹ is a single bond, a methylene group, an ethylene group, a phenylene group, a fluorinated phenylene group, a trifluoromethyl-substituted phenylene group, *—C(═O)—O—Z⁹¹—, *—C(═O)—N(H)—Z⁹¹—, or *—O—Z⁹¹—, Z⁹¹ is a C₁-C₆ aliphatic hydrocarbylene group, a phenylene group, a fluorinated phenylene group, or a trifluoromethyl-substituted phenylene group, which may contain a carbonyl group, an ester bond, an ether bond, or a hydroxy group,
   * designates a point of attachment to the carbon atom in the backbone, ** designates a point of attachment to Z¹, *** designates a point of attachment to Z⁶, **** designates a point of attachment to Z⁷,
   L¹ is a single bond, an ether bond, an ester bond, a carbonyl group, a sulfonate ester bond, a sulfonamide bond, a carbonate bond, or a carbamate bond,
   Rf¹ and Rf² are each independently a fluorine atom or a C₁-C₆ fluorinated saturated hydrocarbyl group,
   Rf³ and Rf⁴ are each independently a hydrogen atom, a fluorine atom, or a C₁-C₆ fluorinated saturated hydrocarbyl group,
   Rf⁵ and Rf⁶ are each independently a hydrogen atom, a fluorine atom, or a C₁-C₆ fluorinated saturated hydrocarbyl group, excluding that all Rf⁵ and Rf⁶ are hydrogen atoms at the same time,
   Rf⁷ is a fluorine atom, a C₁-C₆ fluorinated alkyl group, a C₁-C₆ fluorinated alkoxy group, or a C₁-C₆ fluorinated alkylthio group,
   R³¹ and R³² are each independently a C₁-C₂₀ hydrocarbyl group which may contain a heteroatom, R³¹ and R³² may bond together to form a ring with the sulfur atom to which they are attached,
   R³³ is a halogen atom other than a fluorine atom or a C₁-C₂₀ hydrocarbyl group which may contain a heteroatom, when e3 is 2, 3, or 4, a plurality of R³³ may be identical or different, and the plurality of R³³ may bond together to form a ring with the carbon atoms to which they are attached,
   M⁻ is a non-nucleophilic counter ion, and
   A⁺ is an onium cation.
14. The chemically amplified resist composition of any one of 10 to 13, further comprising an organic solvent.
15. The chemically amplified resist composition of any one of 10 to 14, further comprising an acid generator capable of generating an acid with pKa≤−2.0.
16. The chemically amplified resist composition of any one of 10 to 15, further comprising a quencher other than the quencher of 9.
17. The chemically amplified resist composition of any one of 10 to 16, further comprising a surfactant.
18. A pattern forming process comprising the steps of applying the chemically amplified resist composition of any one of 10 to 17 onto a substrate to form a resist film thereon, exposing the resist film to high-energy radiation, and developing the exposed resist film in a developer.
19. The pattern forming process of 18 wherein the high-energy radiation is KrF excimer laser radiation, ArF excimer laser radiation, EB, or EUV of wavelength 3 to 15 nm.

### Advantageous Effects of Invention

When a pattern is formed using a chemically amplified resist composition comprising the onium salt of the present invention as a quencher, a resist pattern having a high contrast, high sensitivity, and improved lithography properties including EL, LWR, CDU, or DOF can be formed.

### DESCRIPTION OF THE PREFERRED EMBODIMENT

### Onium Salt

The present invention provides an onium salt having the formula (1).

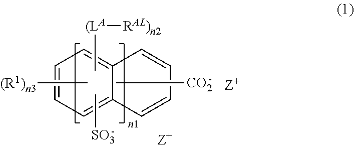

In formula (1), n1 is 0 or 1. The relevant structure is a benzene ring when n1=0, and a naphthalene ring when n1=1. From the viewpoint of solvent solubility, the benzene ring corresponding to n1=0 is preferred. n2 is 1, 2, or 3. From the viewpoint of reactant availability, n2 is preferably 1 or 2. n3 is 0, 1, or 2. From the viewpoint of reactant availability, n3 is preferably 0 or 1. Provided that 1≤n2+n3≤4 when n1 is 0, and 1≤n2+n3≤6 when n1 is 1.

In formula (1), R¹ is a halogen atom, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. Examples of the halogen atom include a fluorine atom, a chlorine atom, a bromine atom, and an iodine atom, and a fluorine atom and an iodine atom are preferred. The hydrocarbyl group may be saturated or unsaturated and straight, branched, or cyclic. Specific examples thereof include C₁-C₂₀ alkyl groups such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and combinations thereof. Among them, aryl groups are preferred. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. Two R¹ may be identical or different when n3 is 2. Two R¹ may bond together to form a ring with the carbon atoms to which they are attached when n3 is 2. The ring is preferably a 5- to 8-membered ring.

In formula (1), R^{AL} is a hydrogen atom or an acid labile group. The acid labile group is preferably a group having the formula (AL-1) or (AL-2).

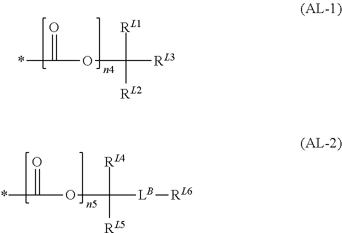

In formula (AL-1), n4 is 0 or 1. R^{L1}, R^{L2}, and R^{L3} are each independently a C₁-C₁₂ hydrocarbyl group in which some —CH₂— may be replaced by —O— or —S—, and when the hydrocarbyl group contains an aromatic ring, some or all of the hydrogen atoms on the aromatic ring may be substituted by a halogen atom, a cyano group, a nitro group, an optionally halogenated C₁-C₄ alkyl group, or an optionally halogenated C₁-C₄ alkoxy group. R^{L1} and R^{L2} may bond together to form a ring with the carbon atom to which they are attached, and some —CH₂— in the ring may be replaced by —O— or —S—.

In formula (AL-2), n5 is 0 or 1. R¹⁴ and R¹⁵ are each independently a hydrogen atom or a C₁-C₁₀ hydrocarbyl group. R^{L6} is a C₁-C₂₀ hydrocarbyl group in which some —CH₂— may be replaced by —O— or —S—. R^{L5} and R^{L6} may bond together to form a C₃-C₂₀ heterocyclic group with the carbon atom and L^{B} to which they are attached, and some —CH₂— in the heterocyclic group may be replaced by —O— or —S—. L^{B} is-O— or —S—.

Specific examples of the acid labile group having formula (AL-1) are shown below. but are not limited thereto. Herein * designates a point of attachment to the adjacent —O—.

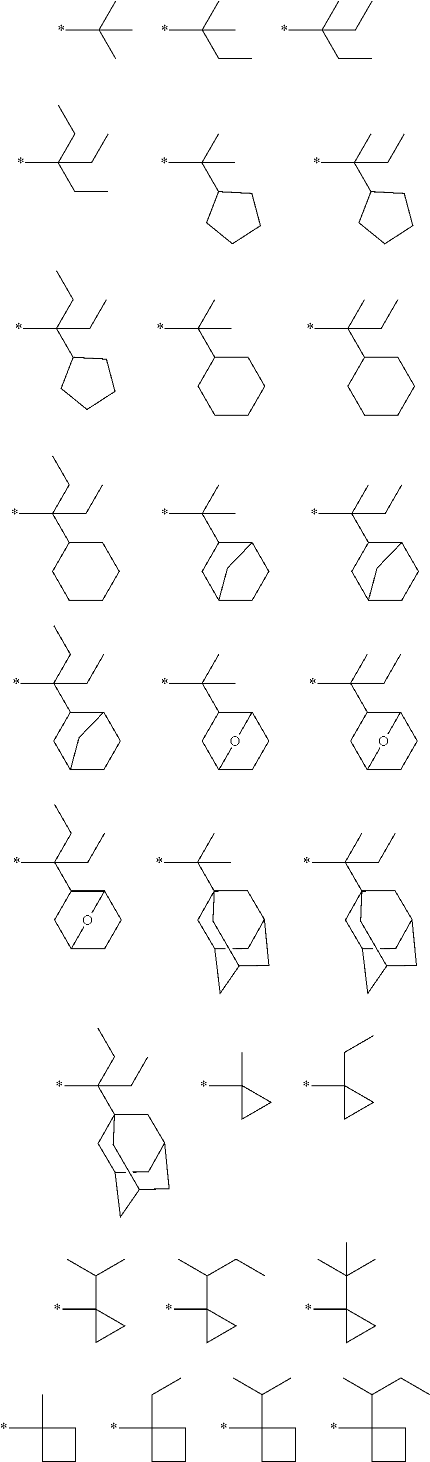

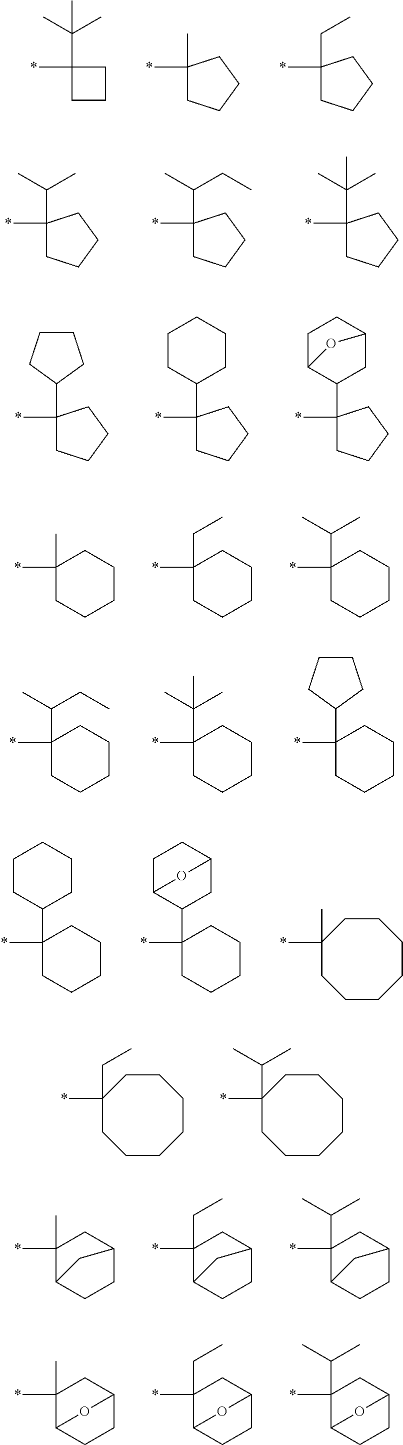

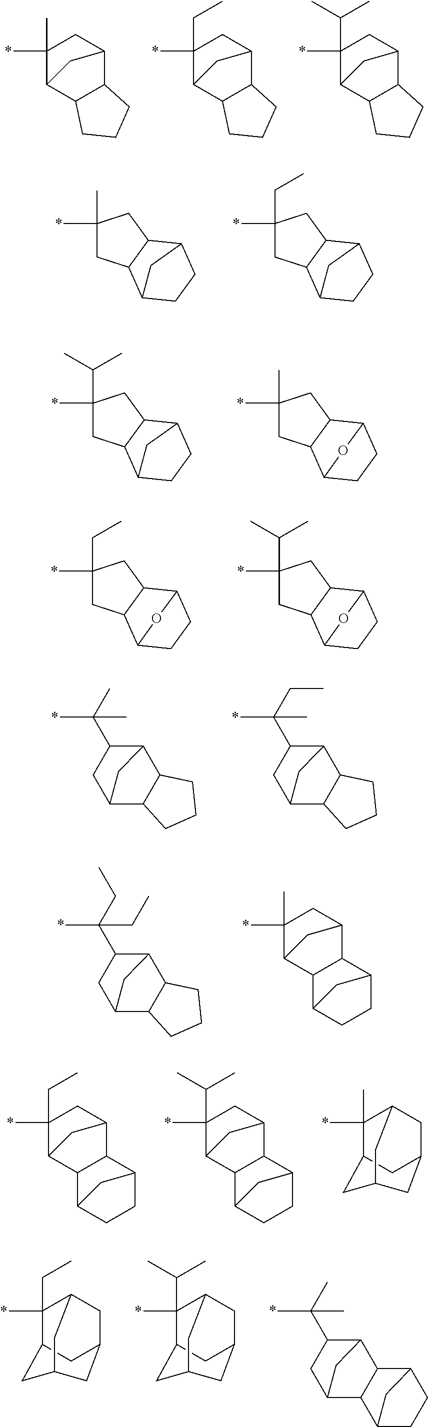

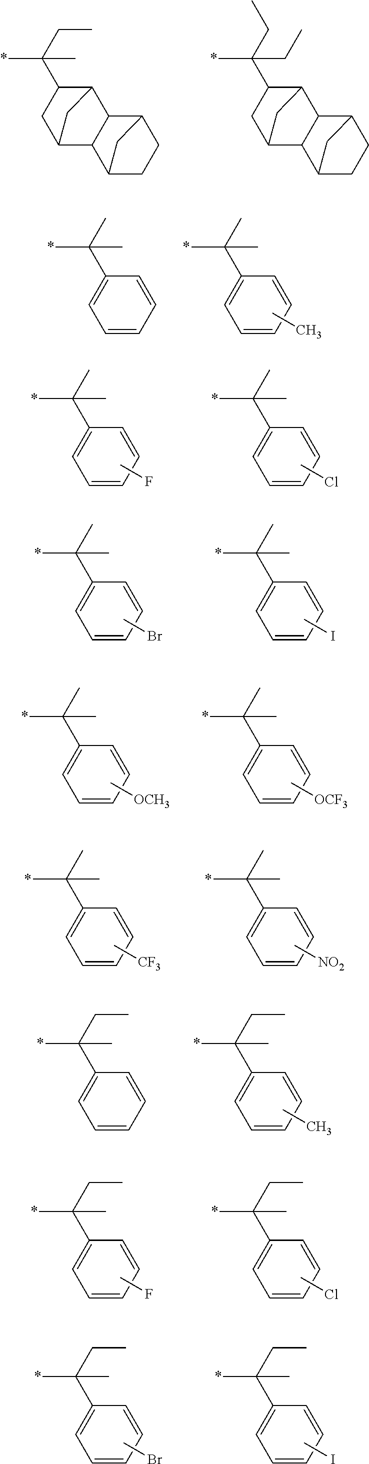

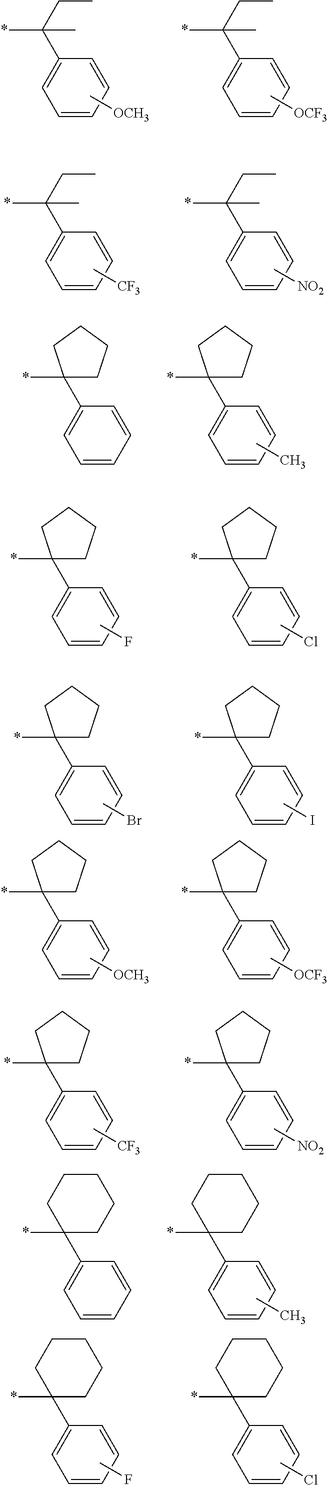

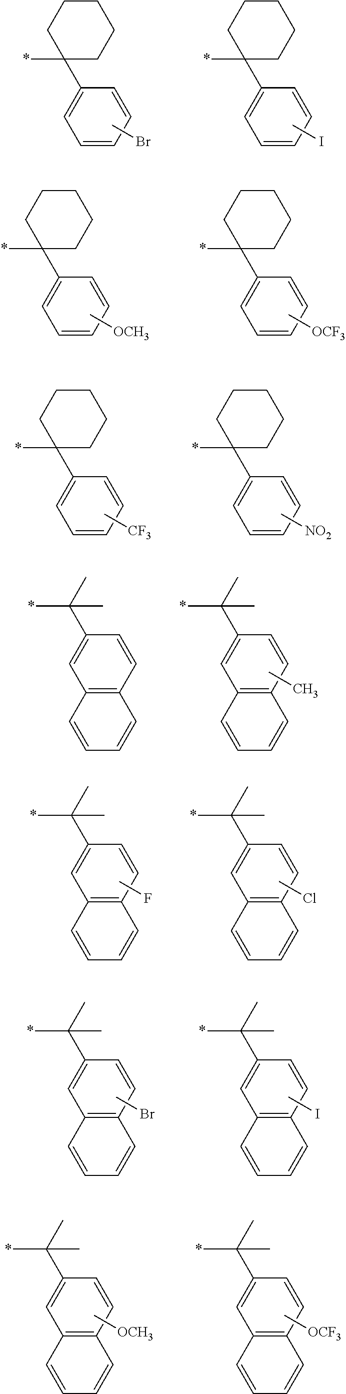

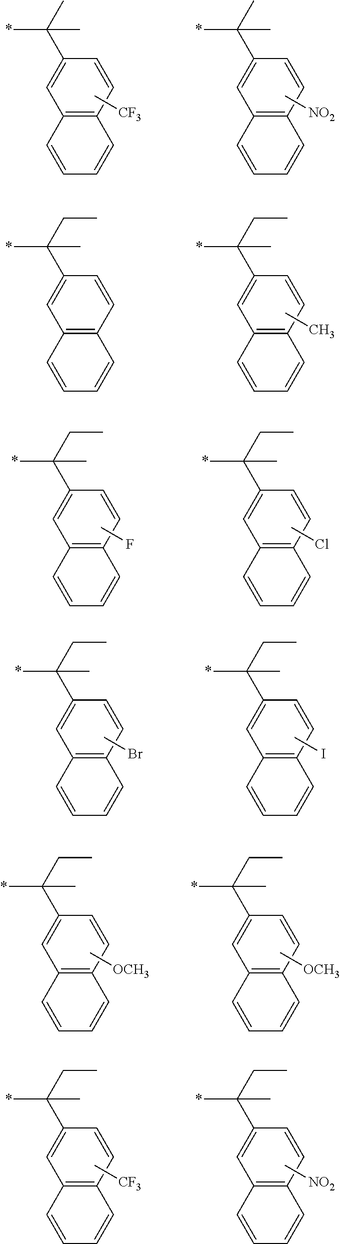

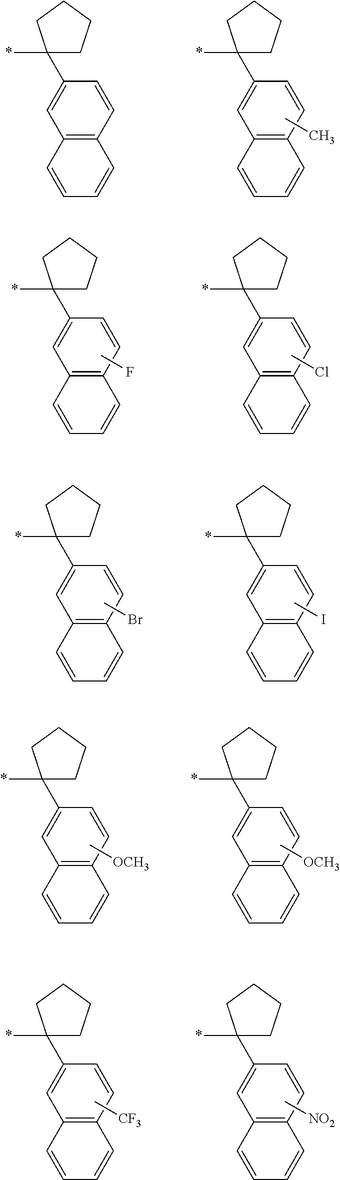

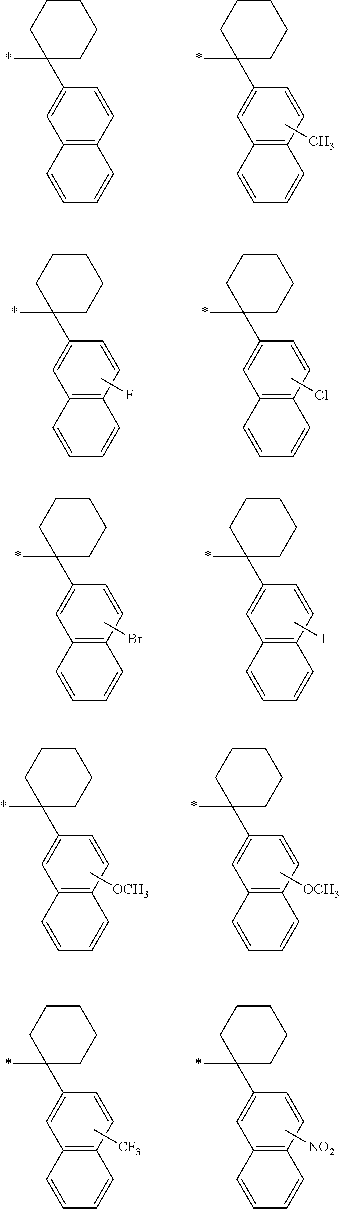

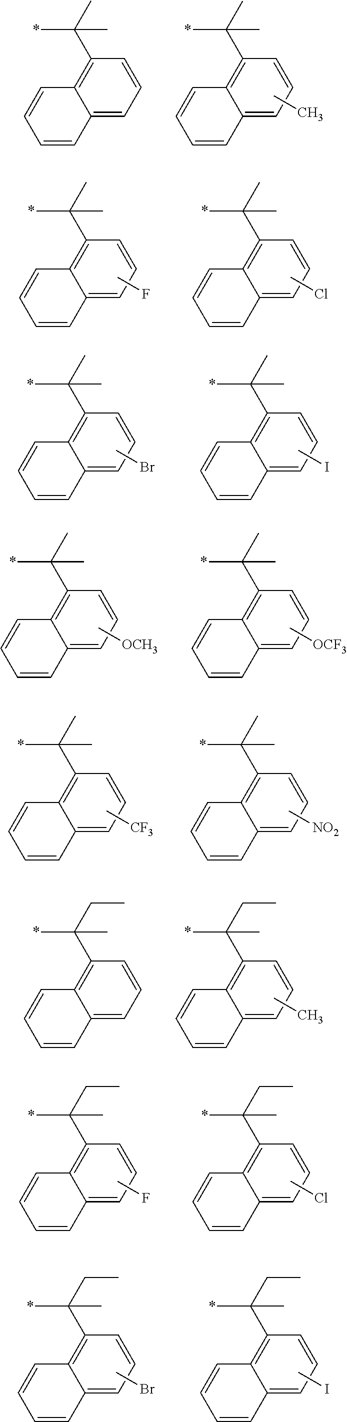

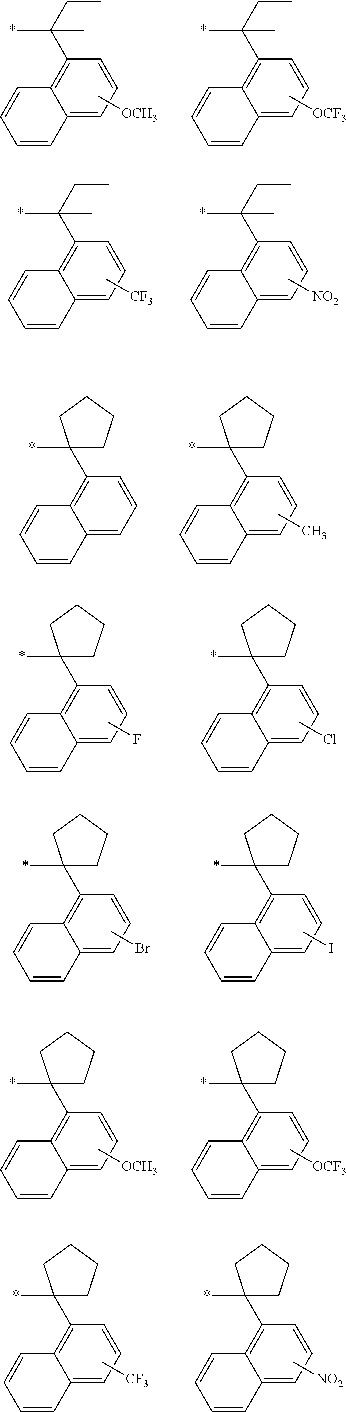

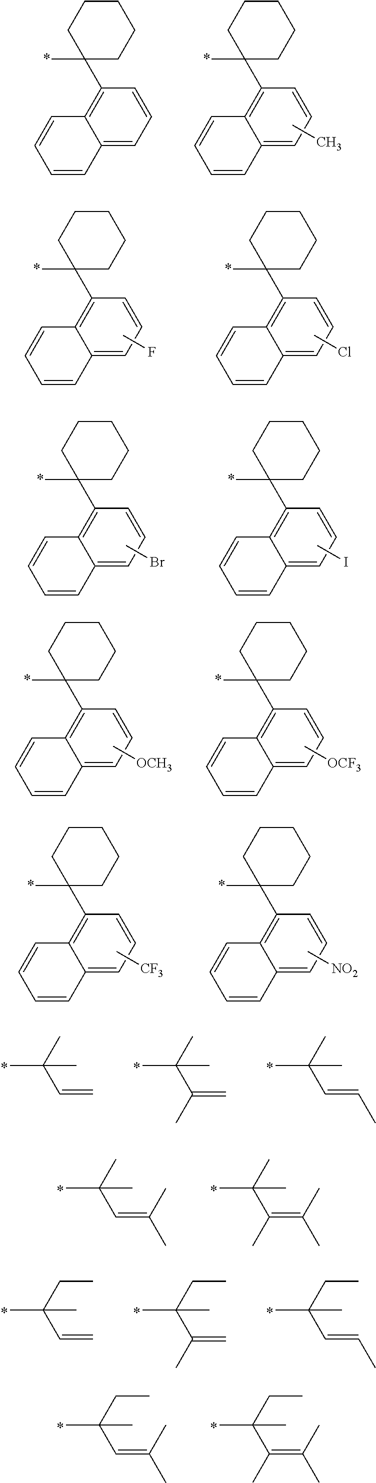

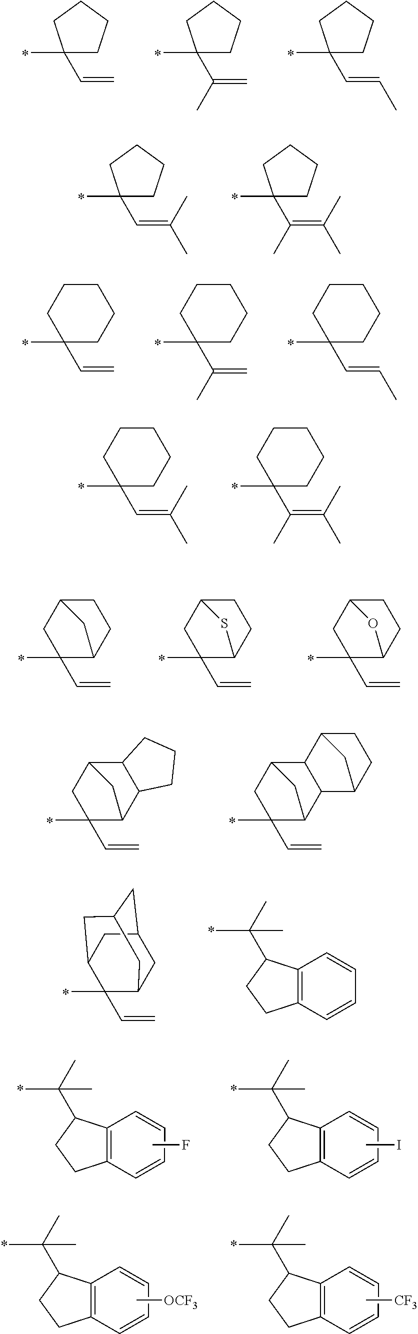

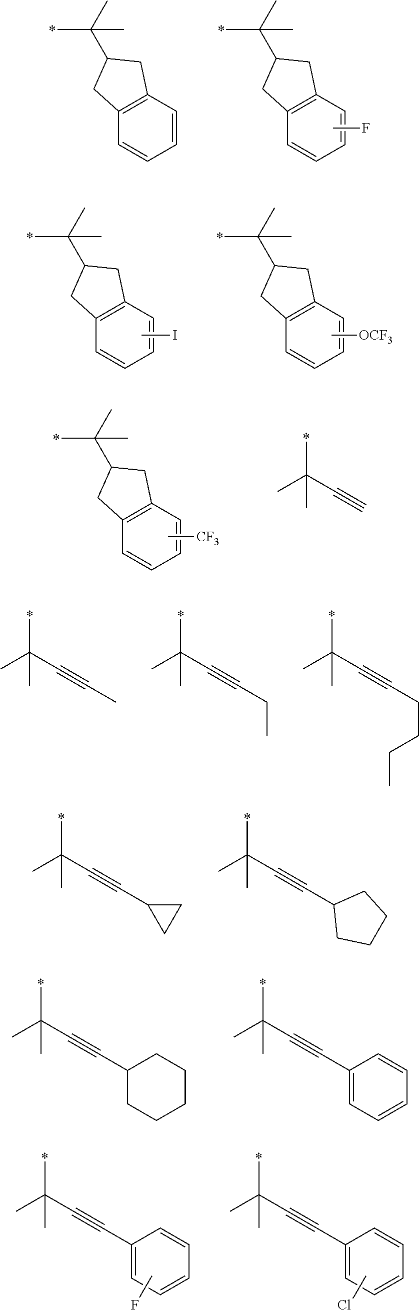

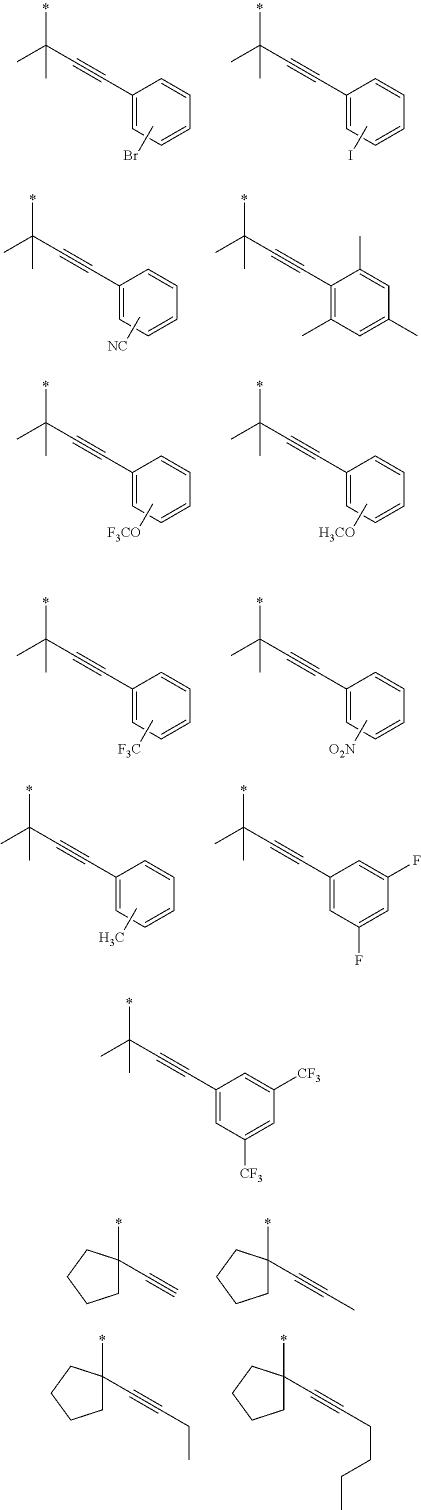

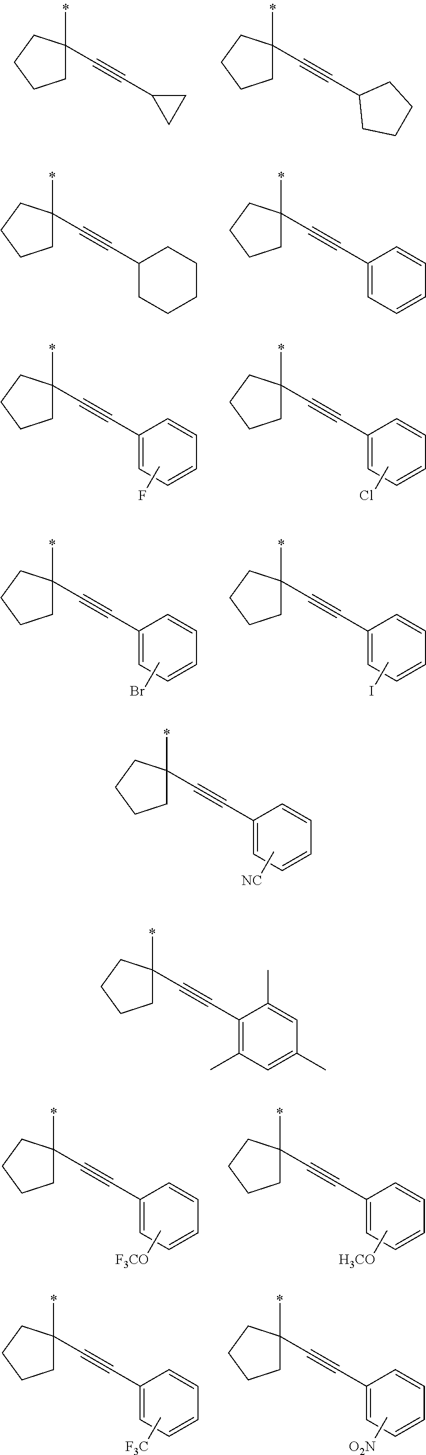

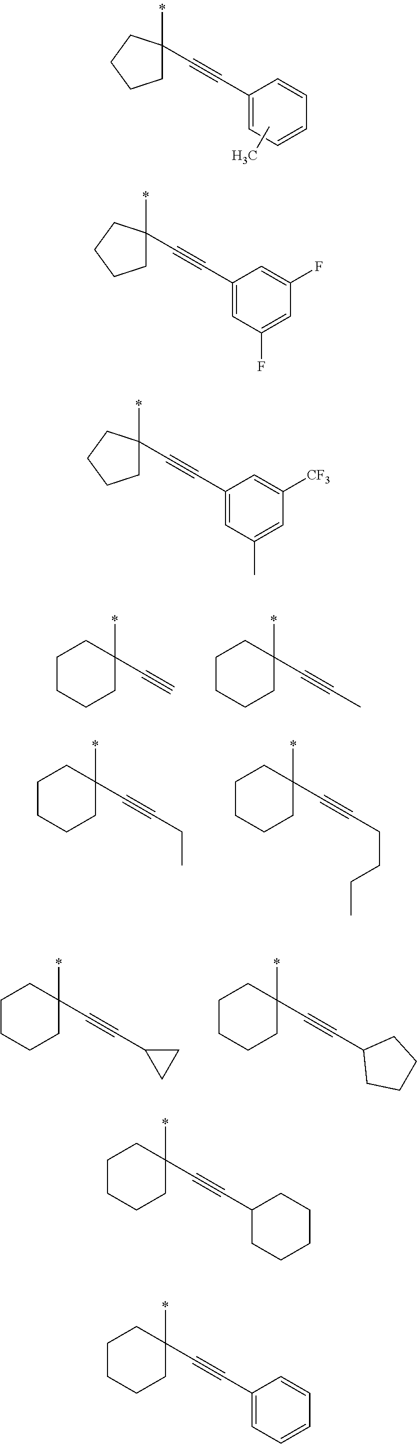

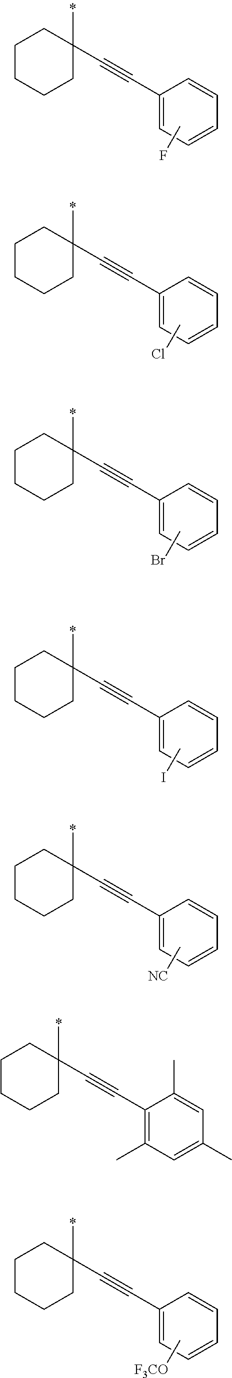

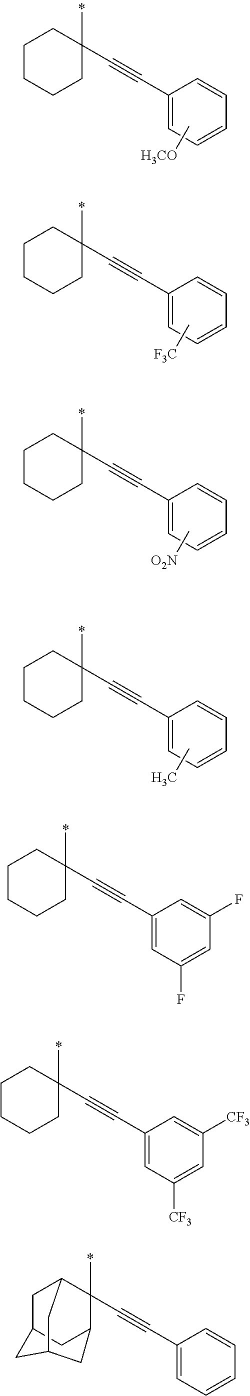

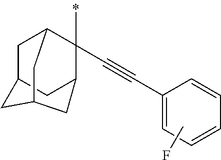

Specific examples of the acid labile group having formula (AL-2) are shown below. but are not limited thereto. Herein * designates a point of attachment to the adjacent —O—.

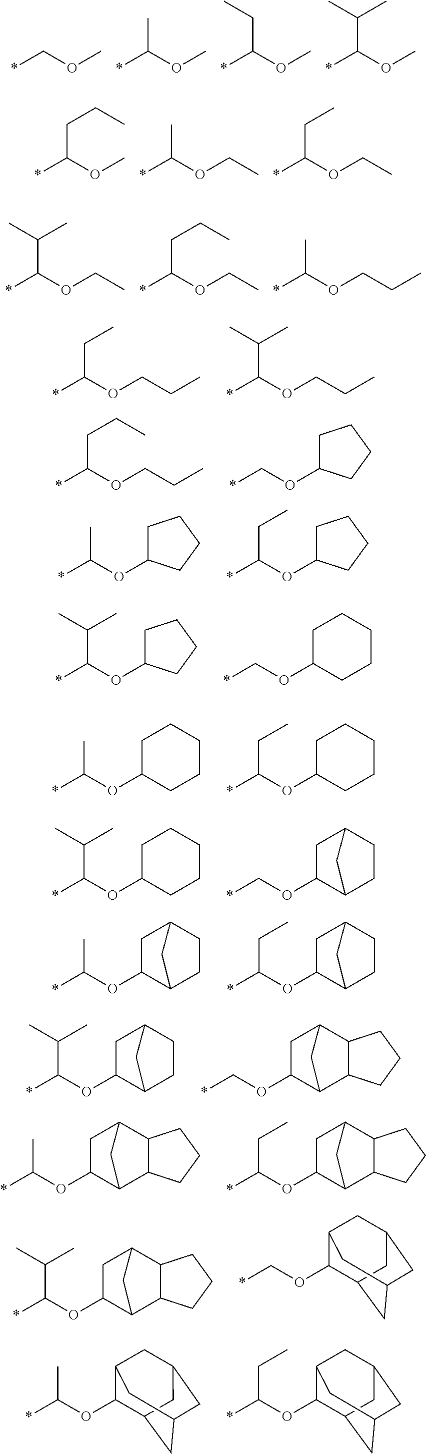

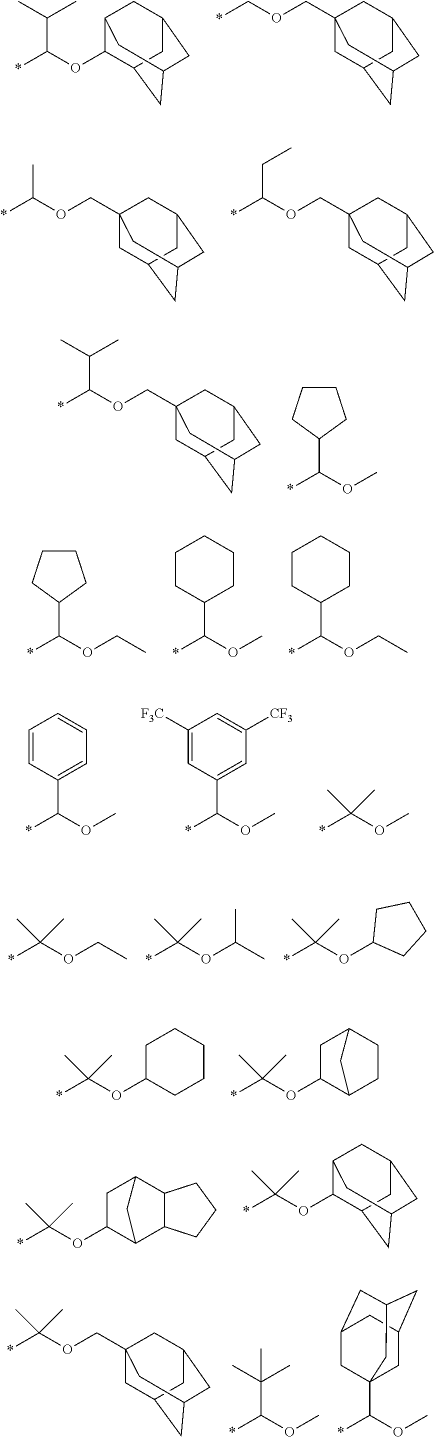

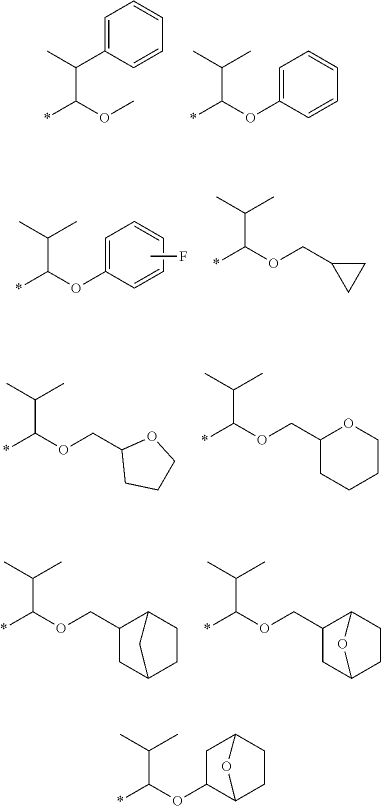

In formula (1), L^{A} is an oxygen atom when R^{AL} is a hydrogen atom, and is an ether bond, an ester bond, or a carbonate bond when R^{AL} is an acid labile group. Among them, an ether bond and an ester bond are preferred, and an ether bond is more preferred.

The onium salt having formula (1) is preferably an onium salt having the formula (1A):

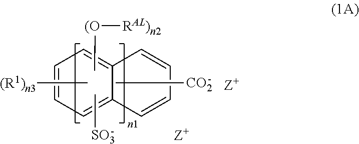

wherein R¹, R^{AL}, n1 to n3, and Z⁺ are as defined above.

The onium salt having formula (1A) is preferably an onium salt having the formula (1B):

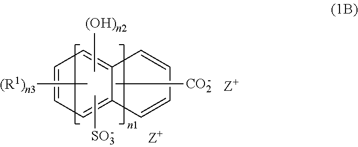

wherein R¹, n1 to n3, and Z⁺ are as defined above.

Examples of the anion of the onium salt having formula (1) are shown below, but are not limited thereto. Herein Me is a methyl group. As for the substitution position of the substituent on the aromatic ring, it is preferable that-O—R^{AL} and —CO₂⁻ are disposed adjacent to each other.

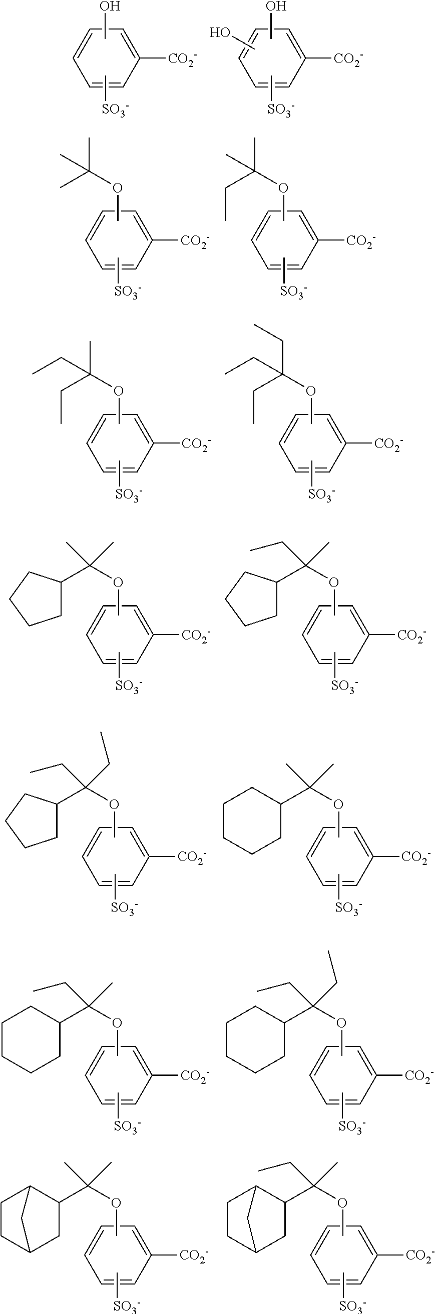

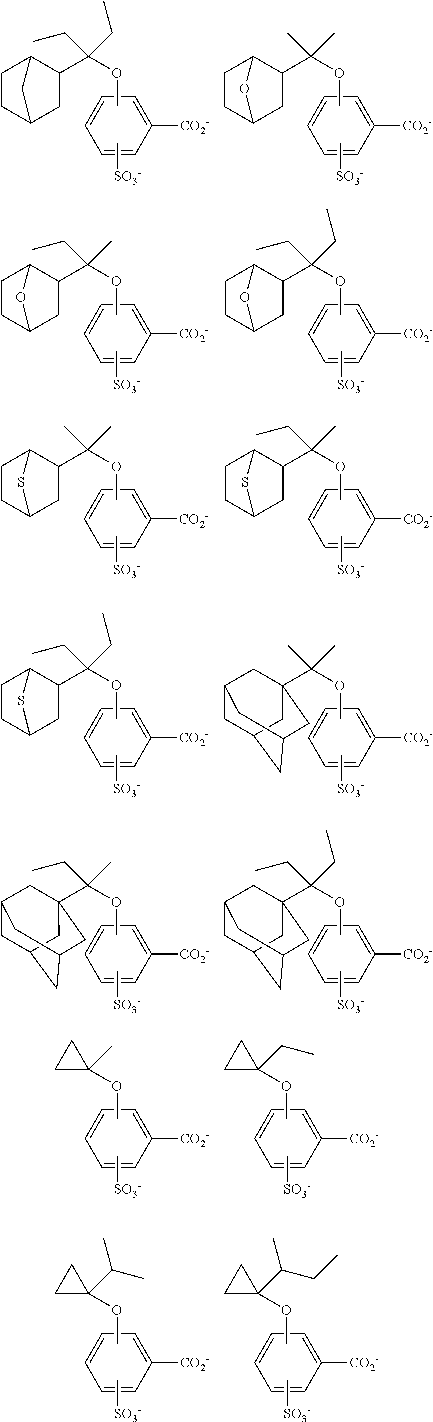

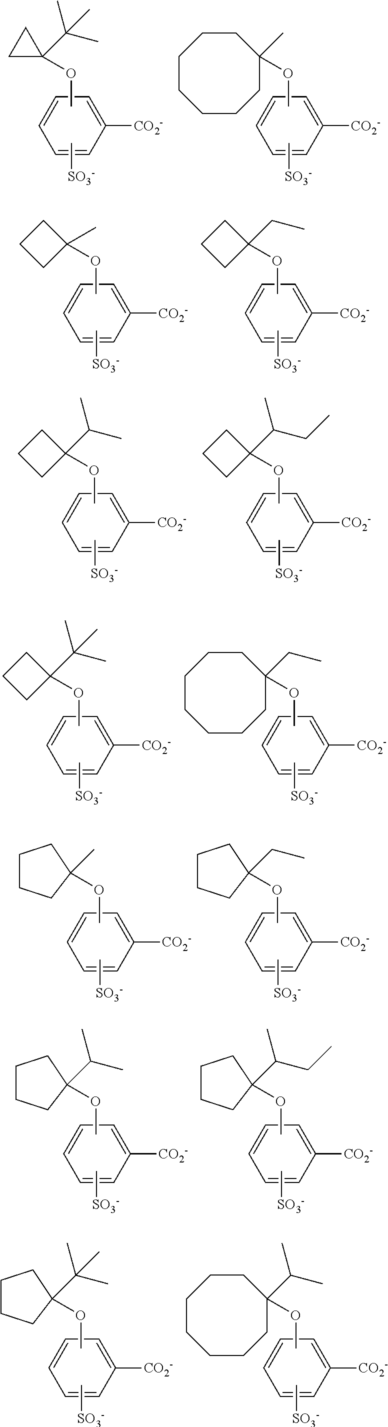

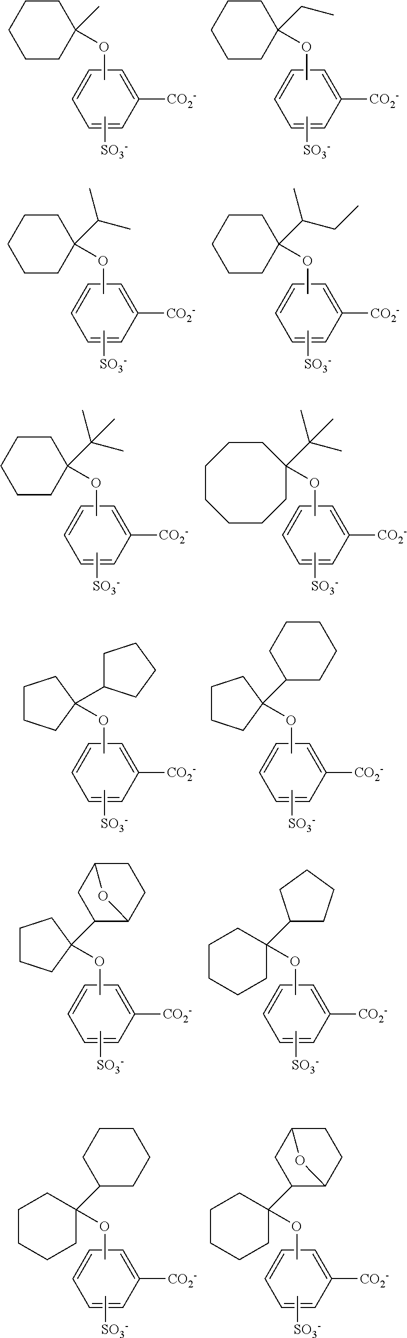

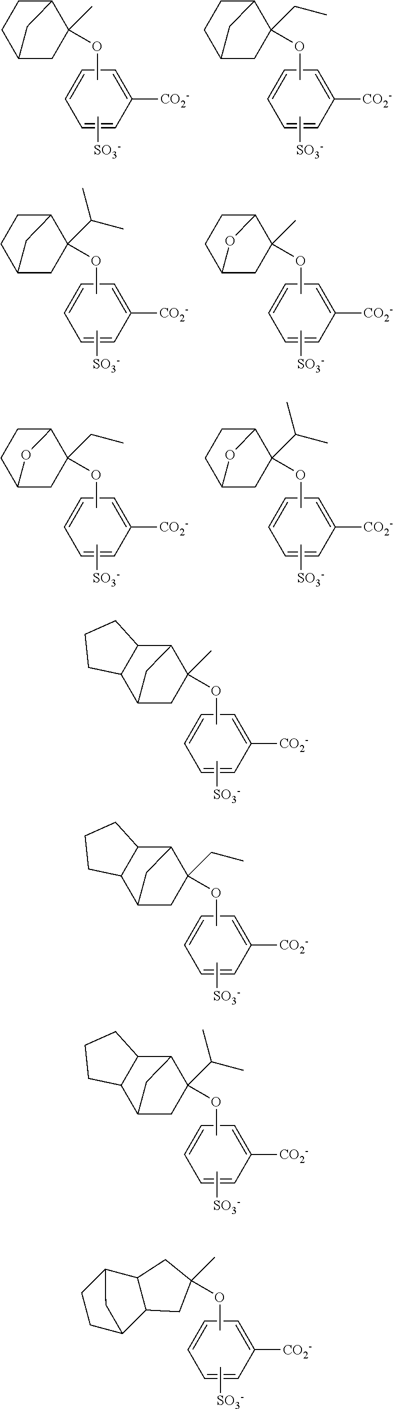

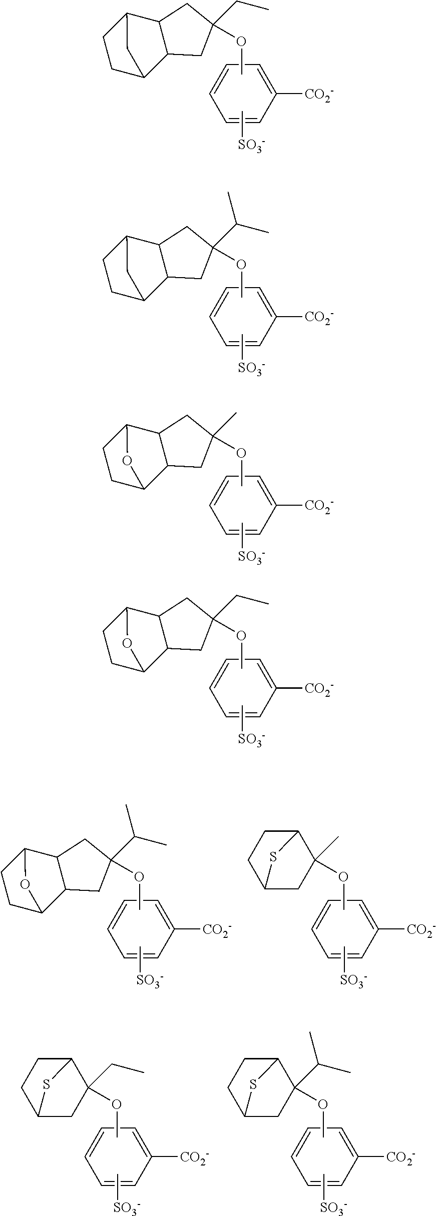

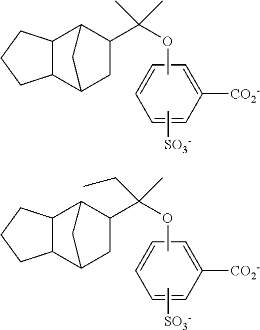

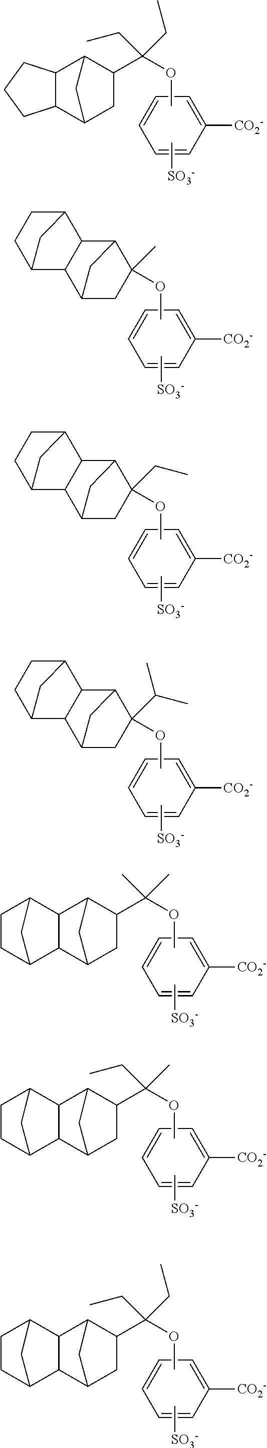

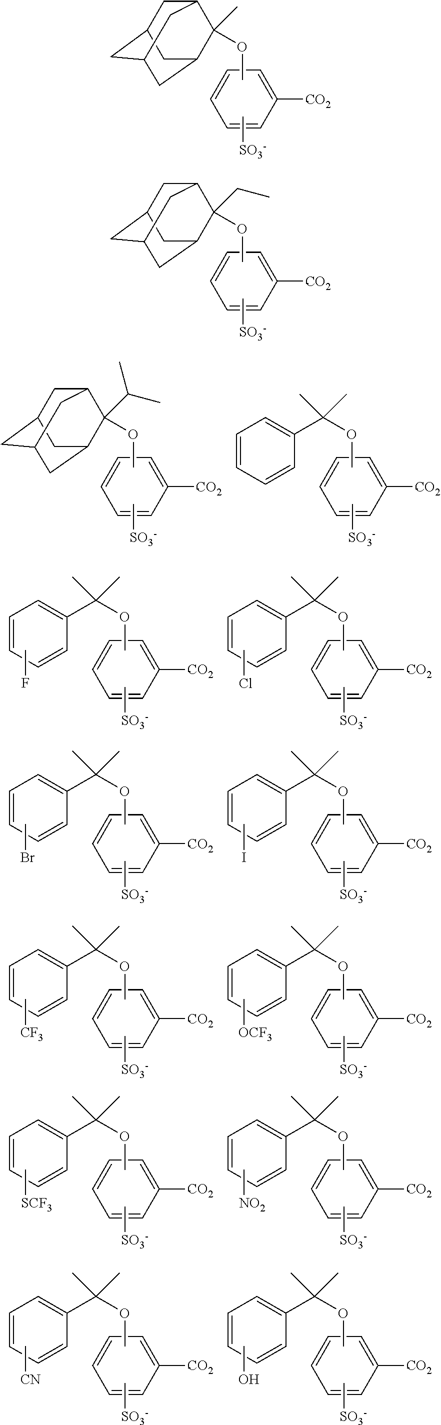

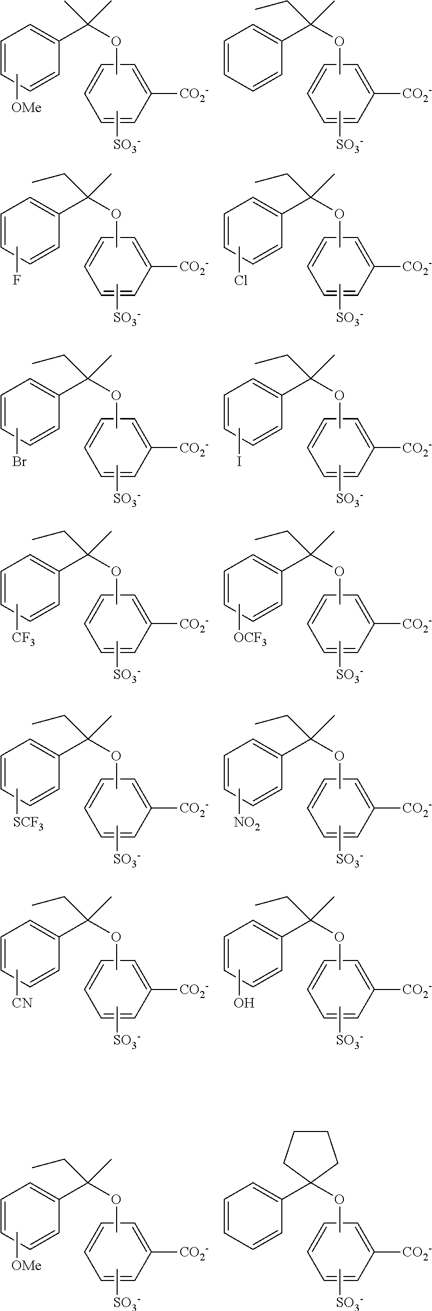

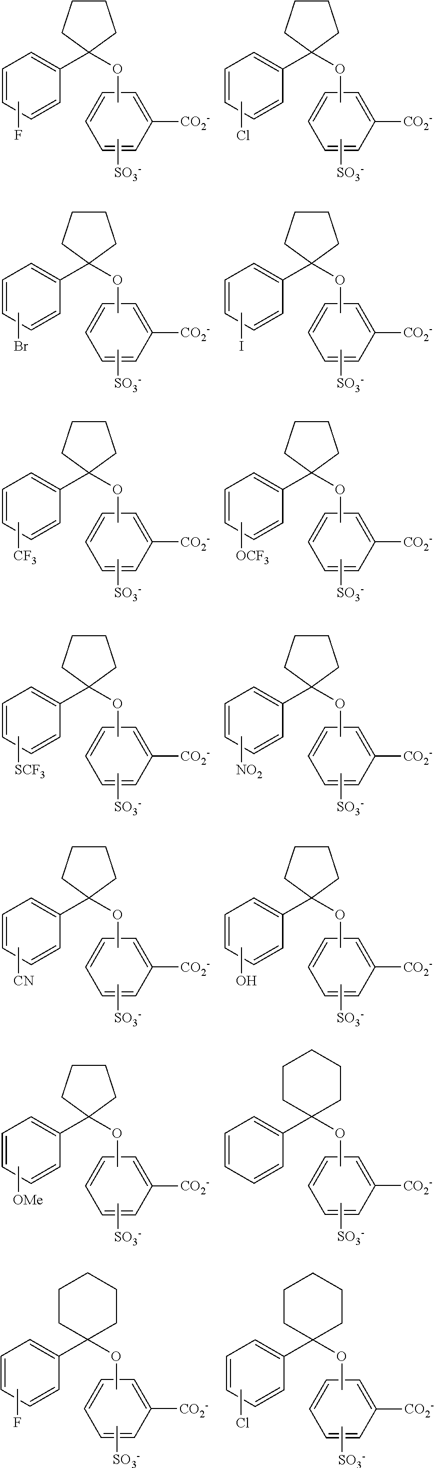

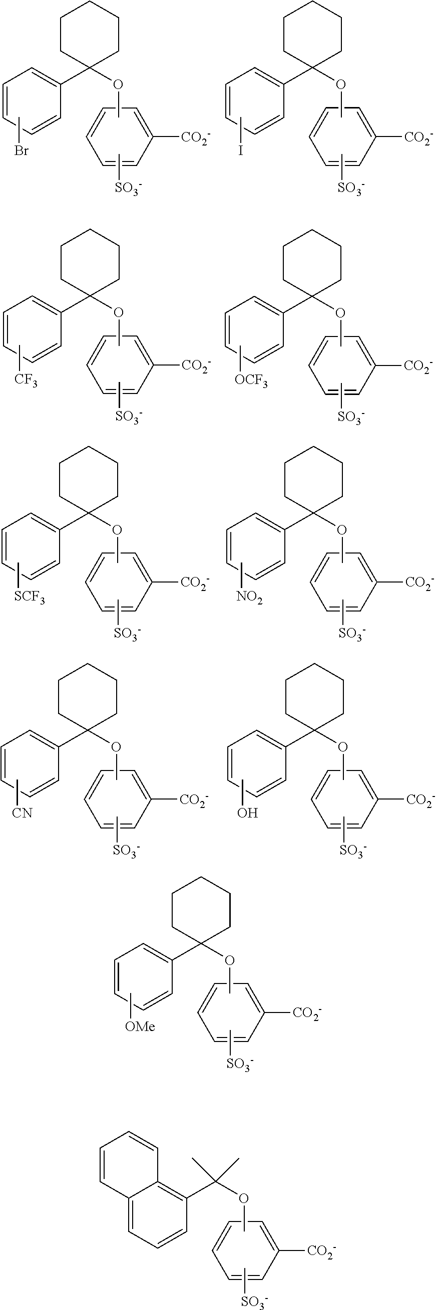

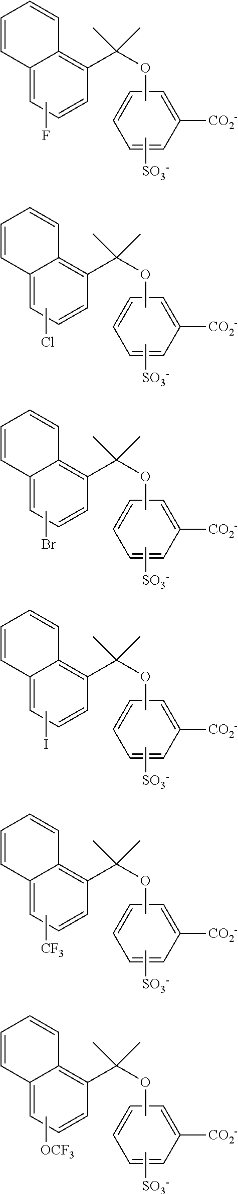

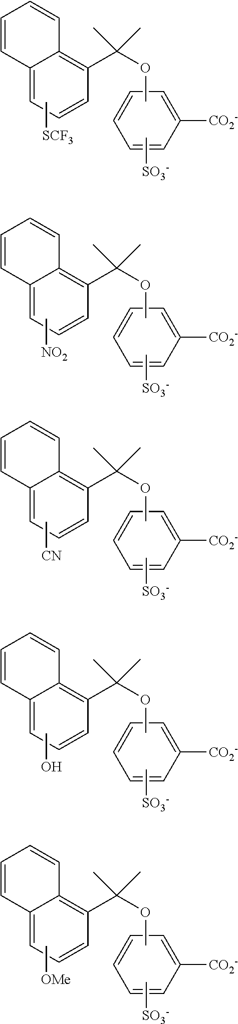

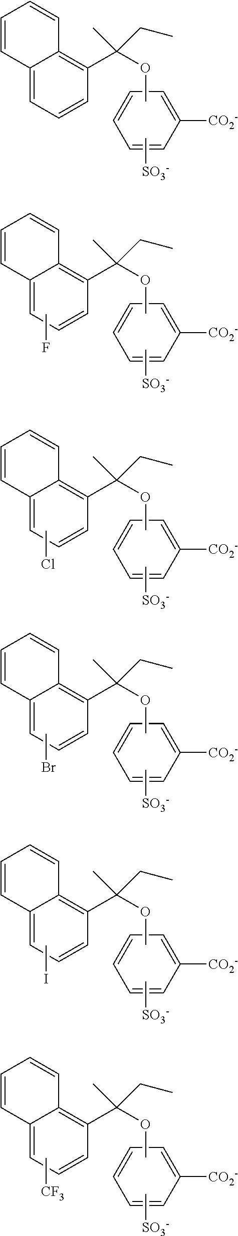

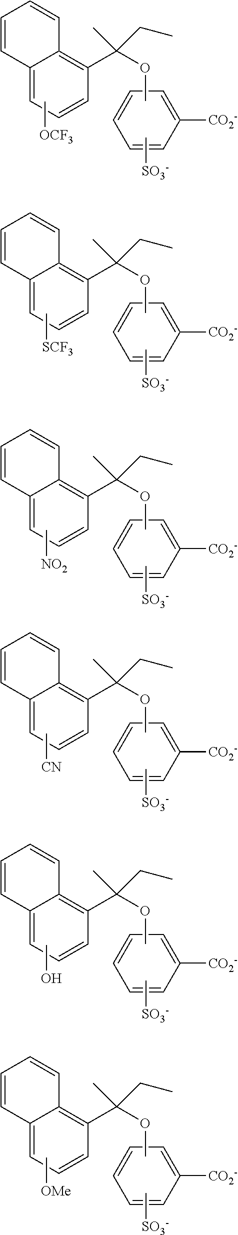

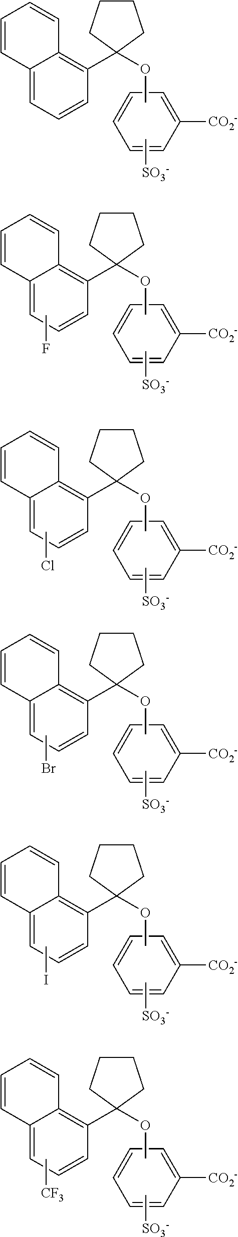

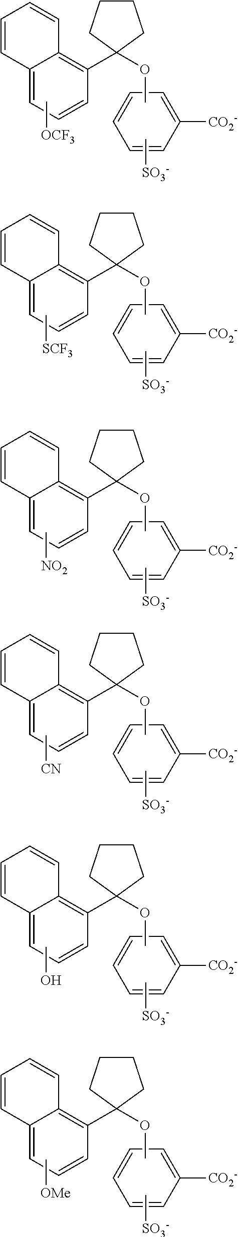

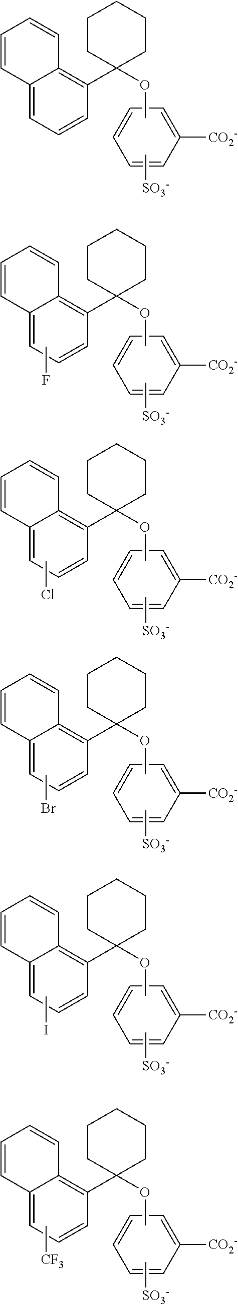

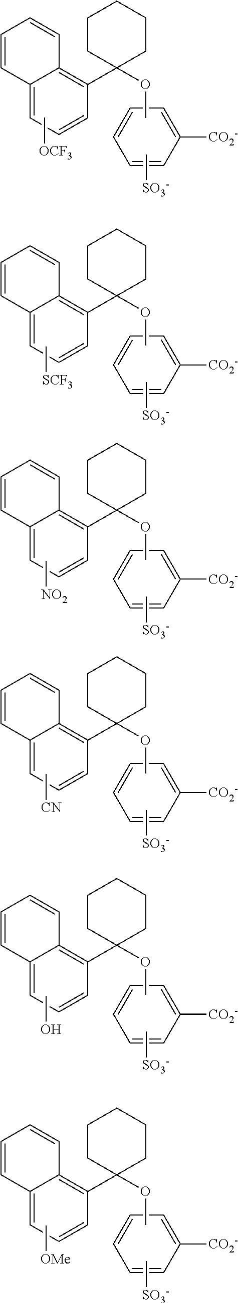

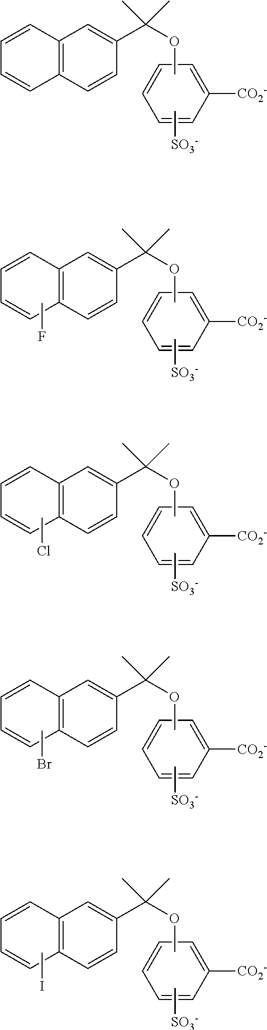

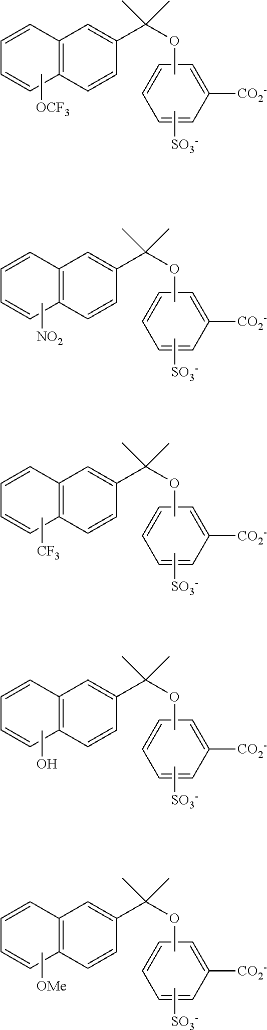

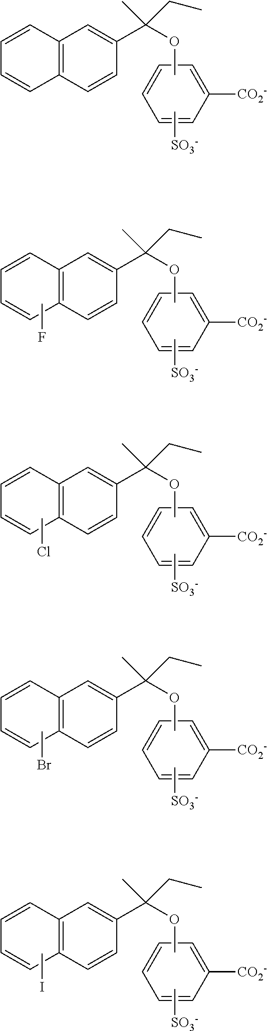

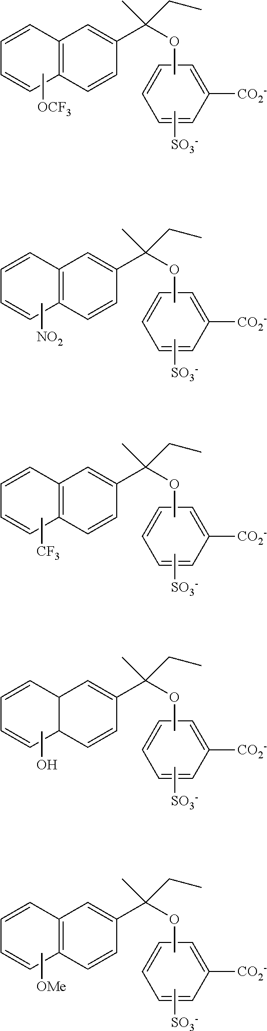

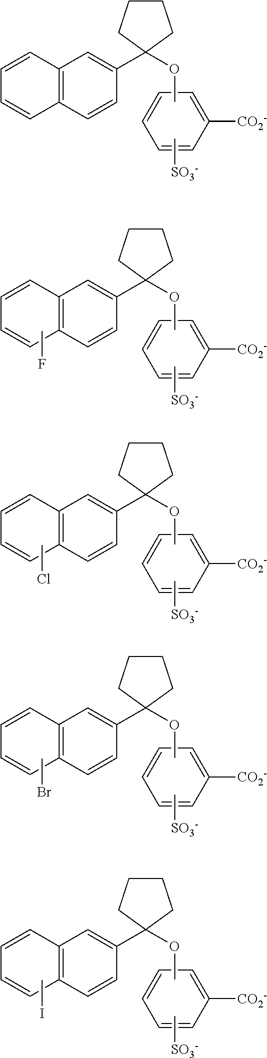

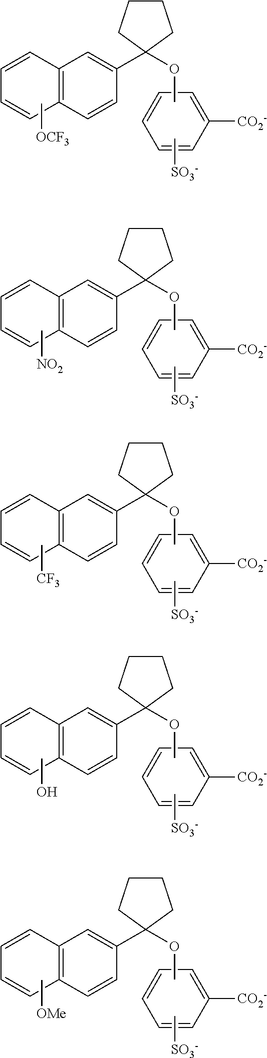

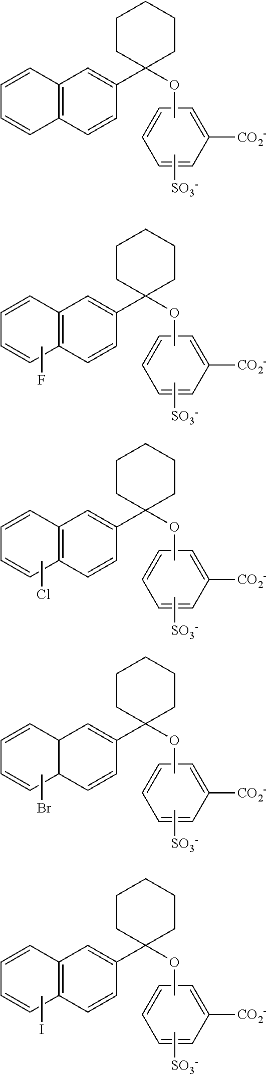

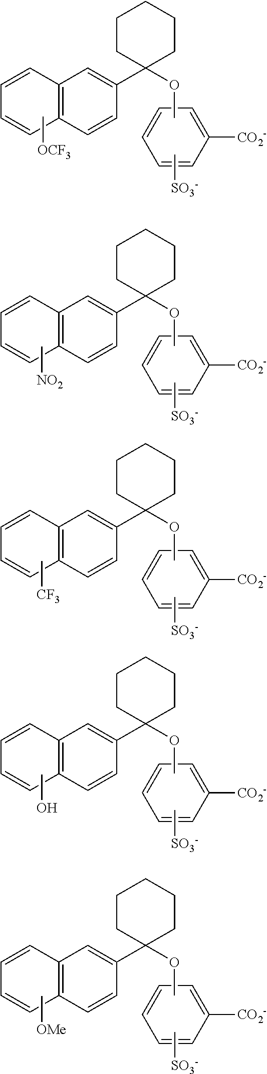

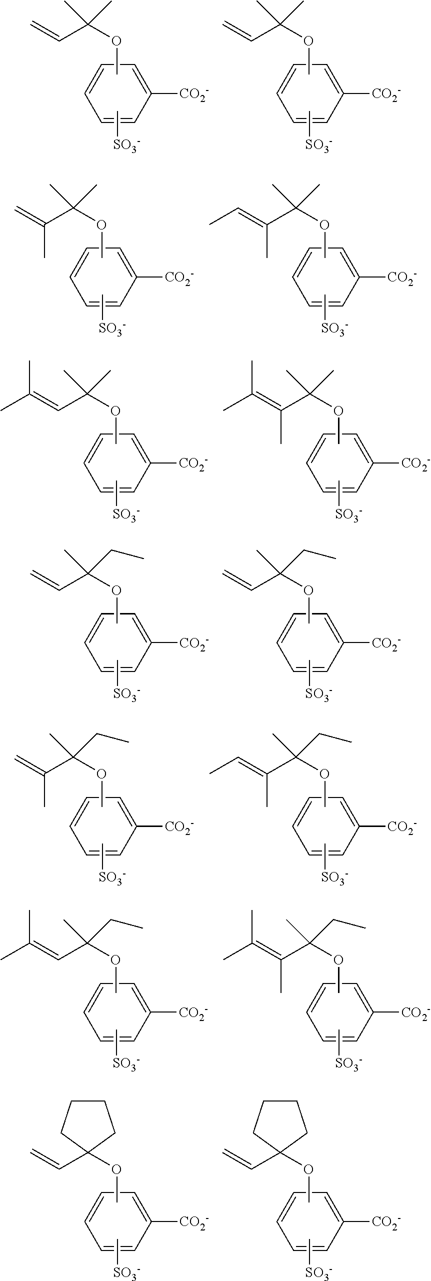

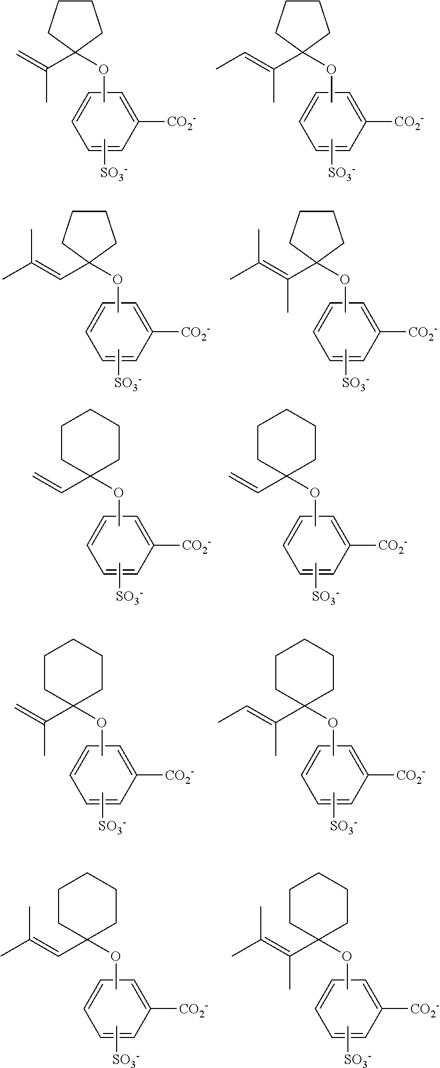

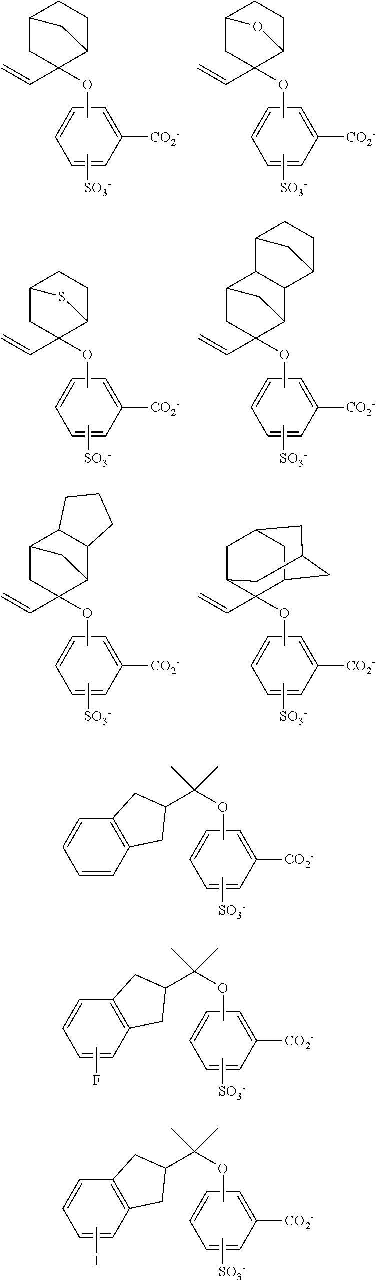

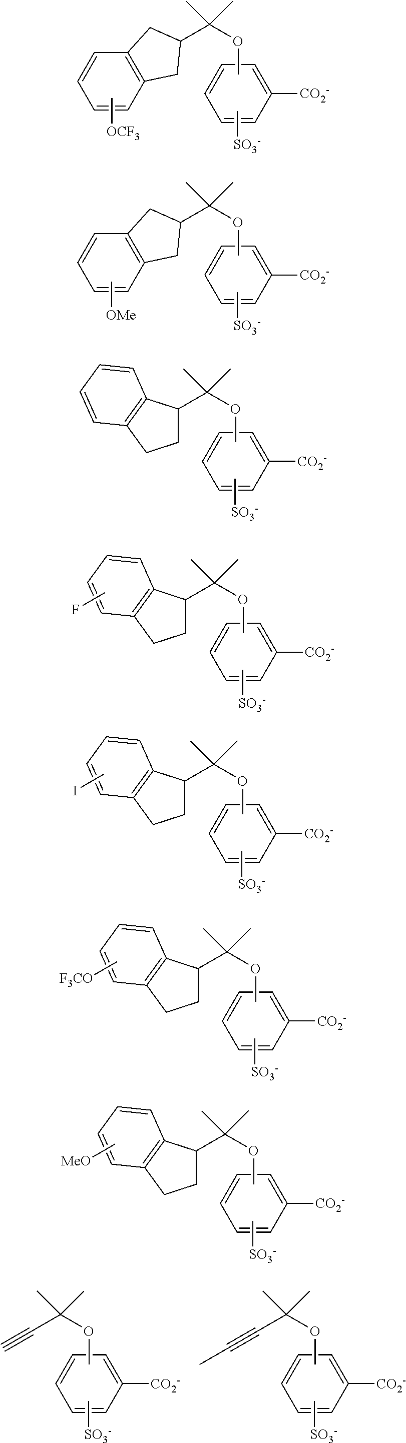

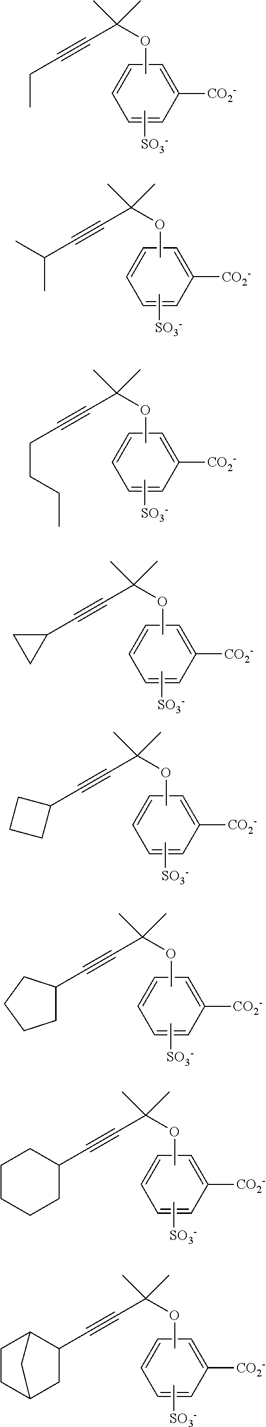

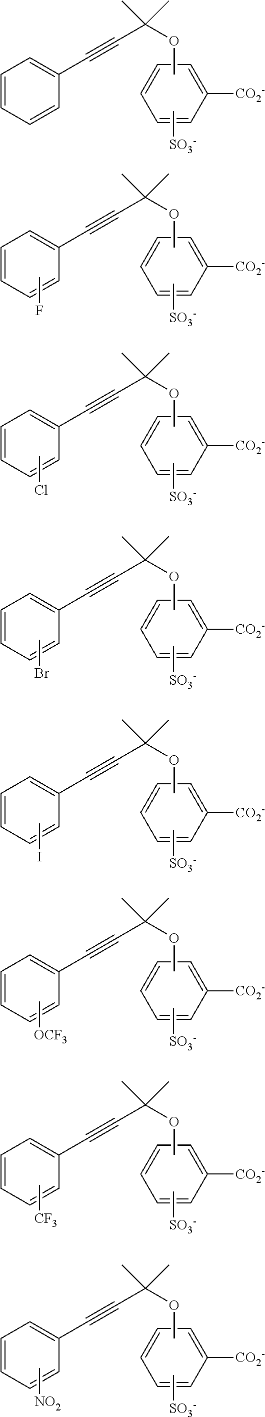

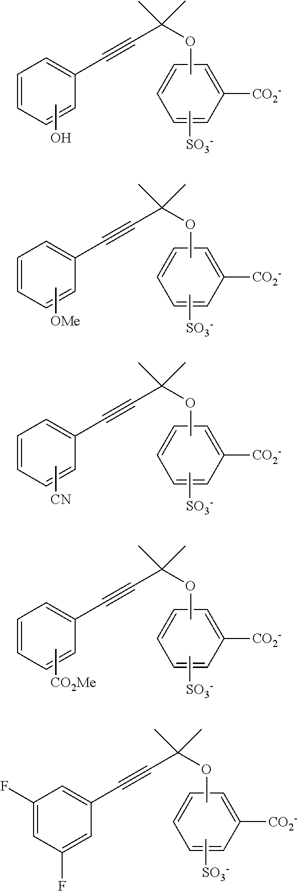

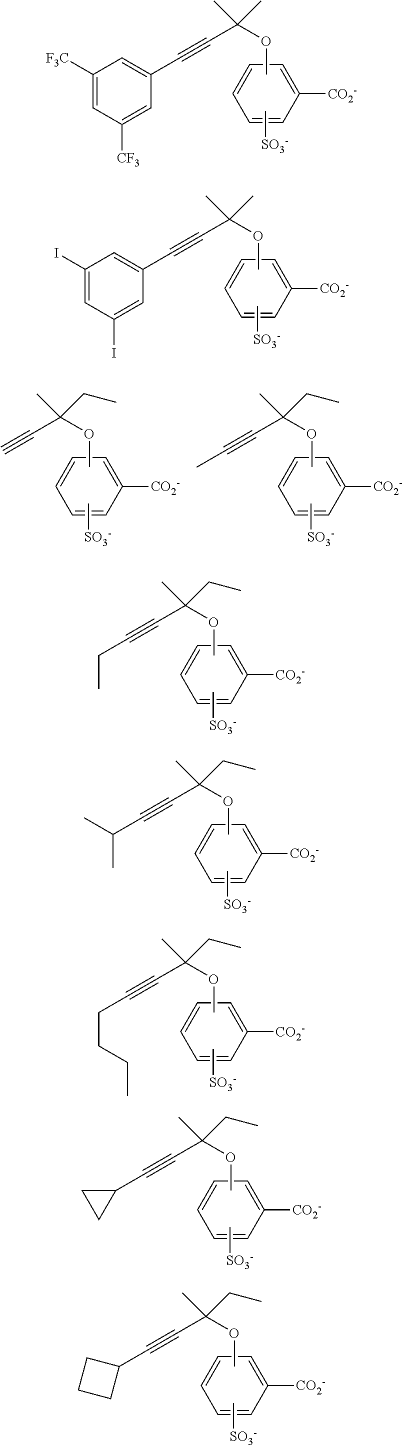

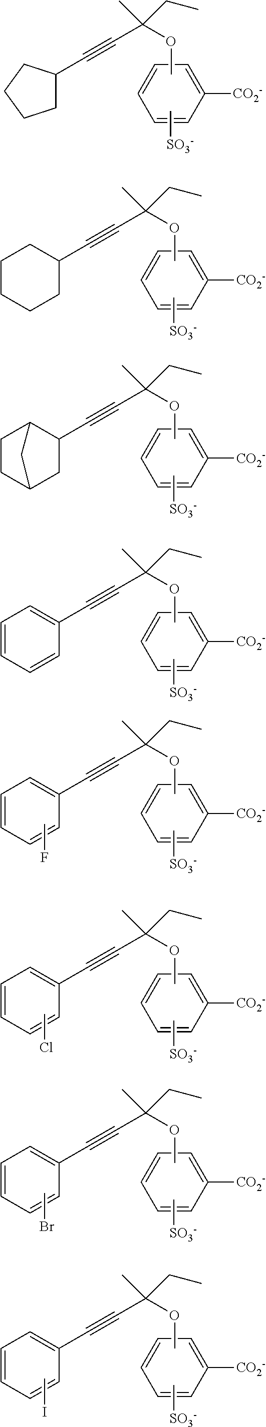

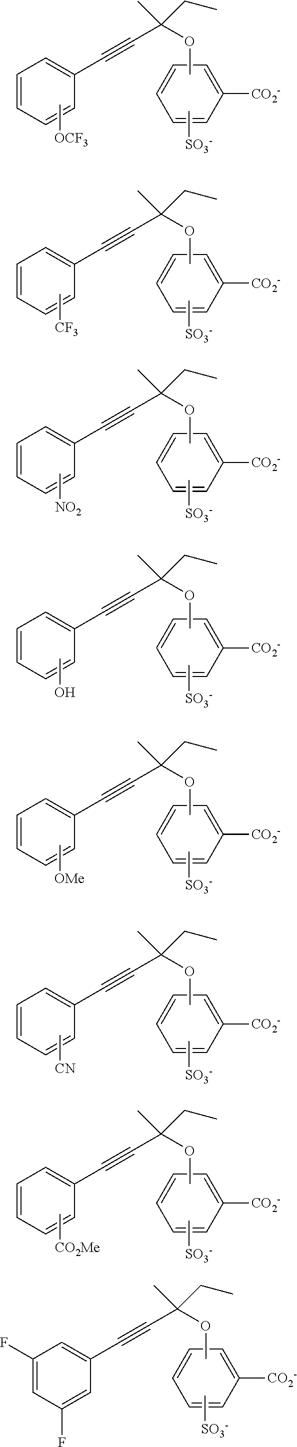

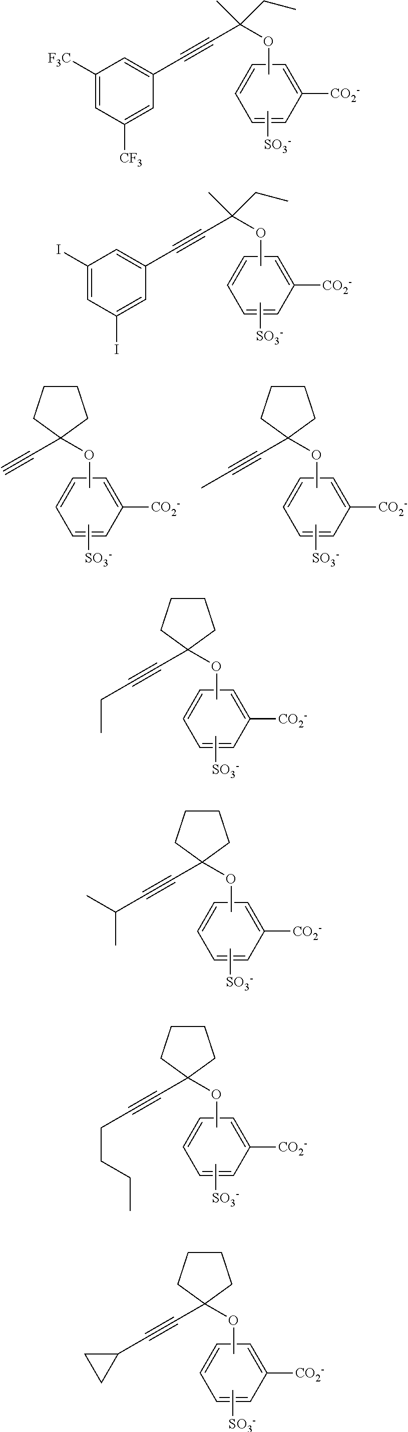

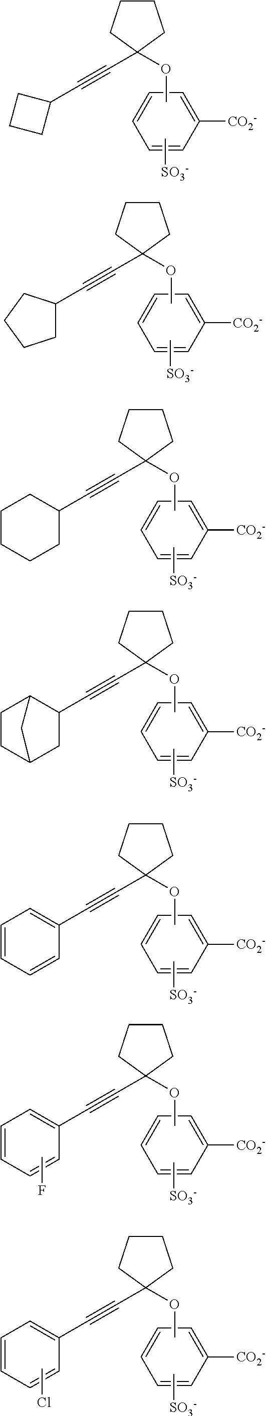

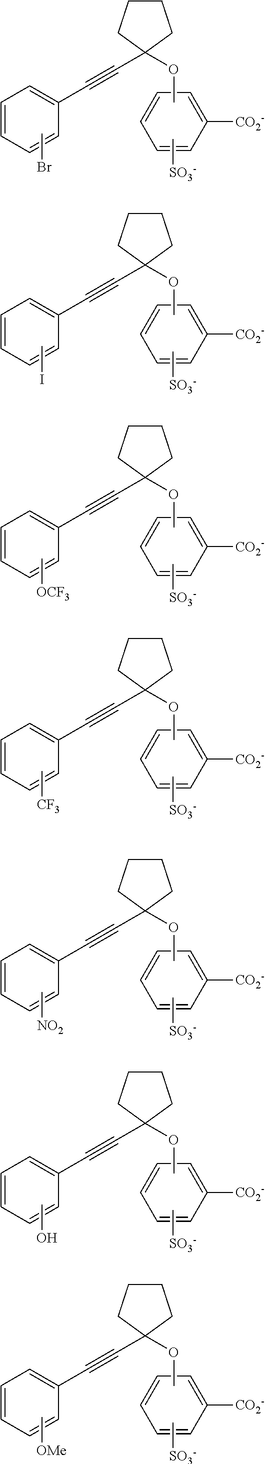

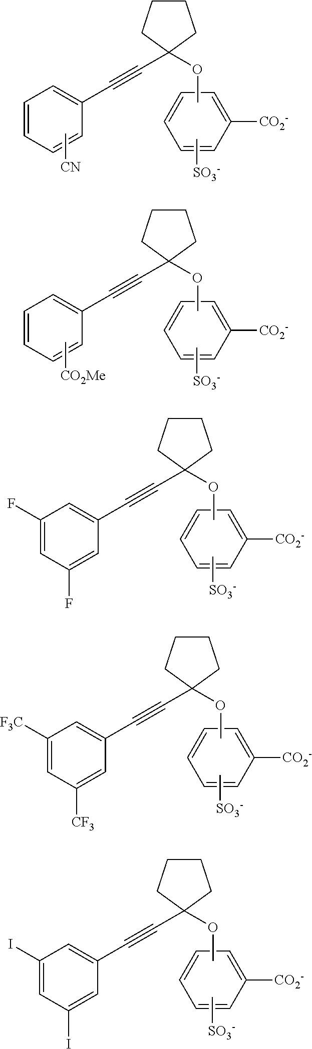

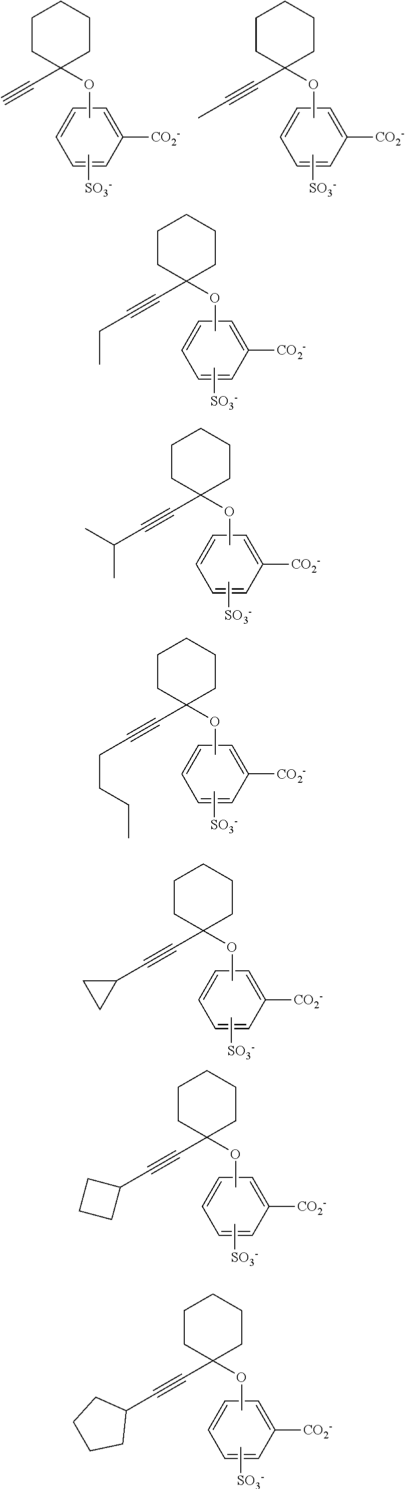

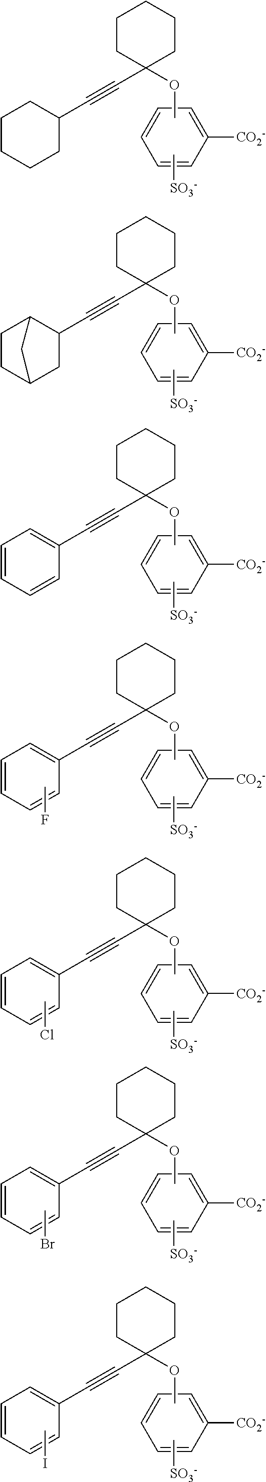

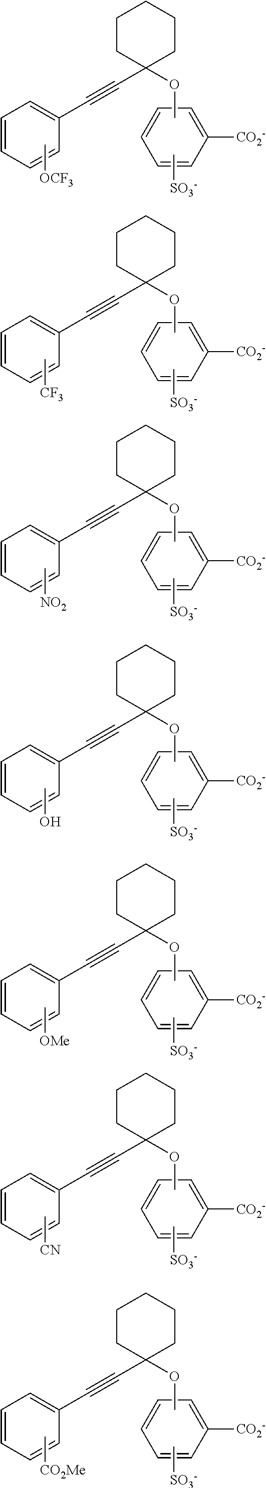

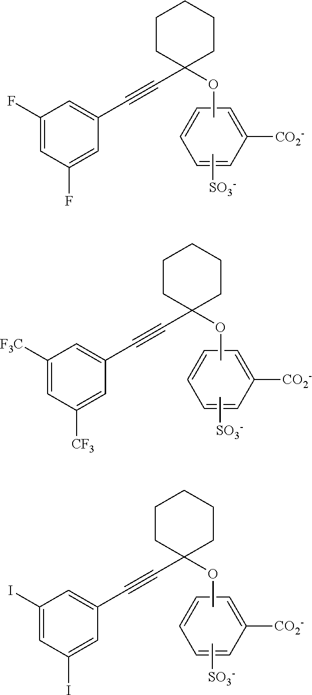

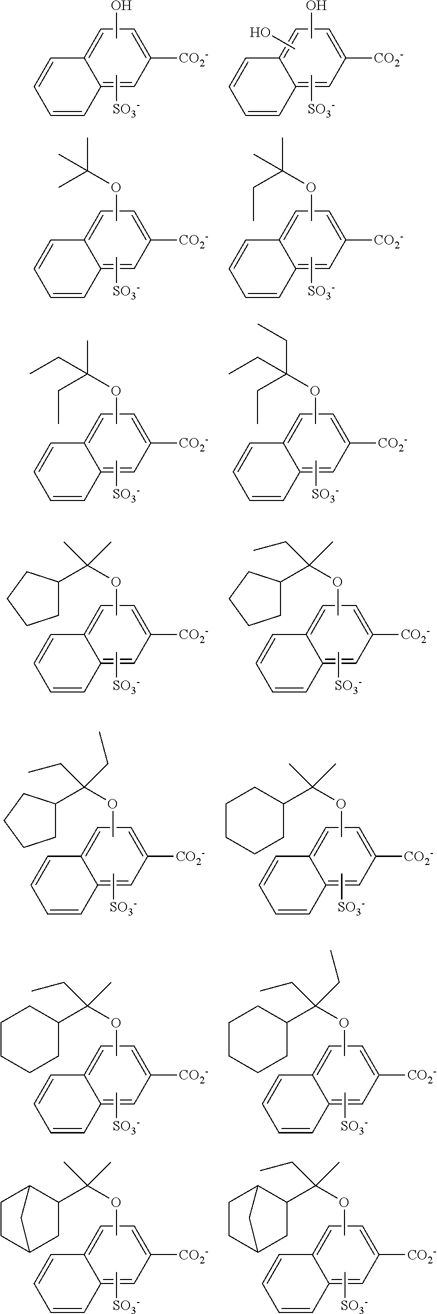

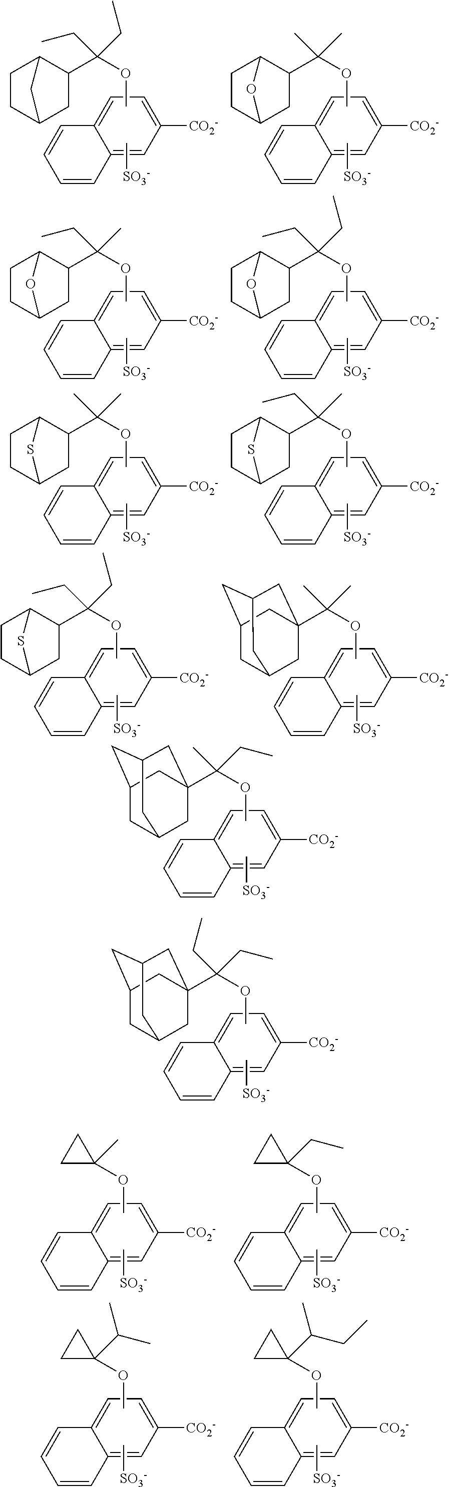

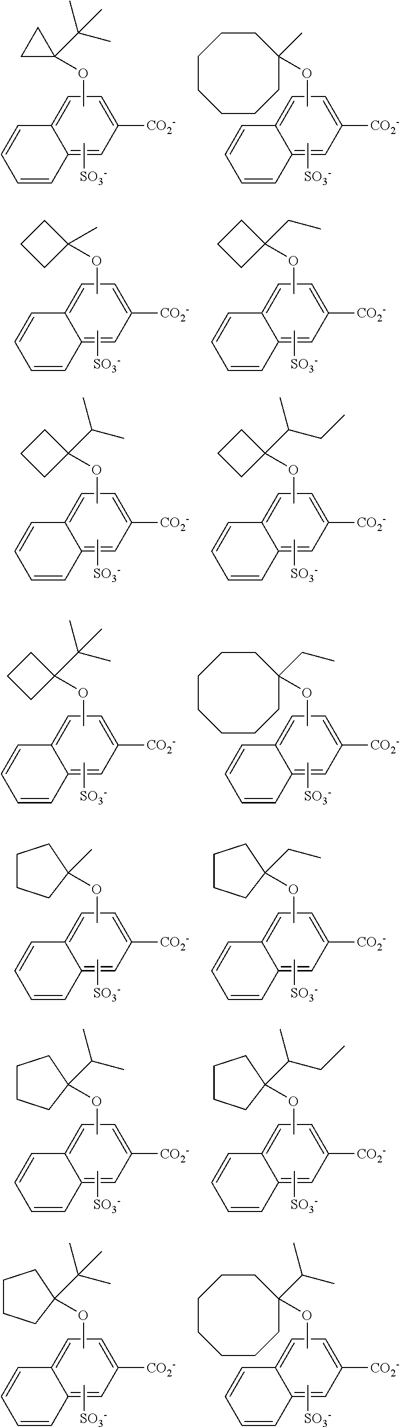

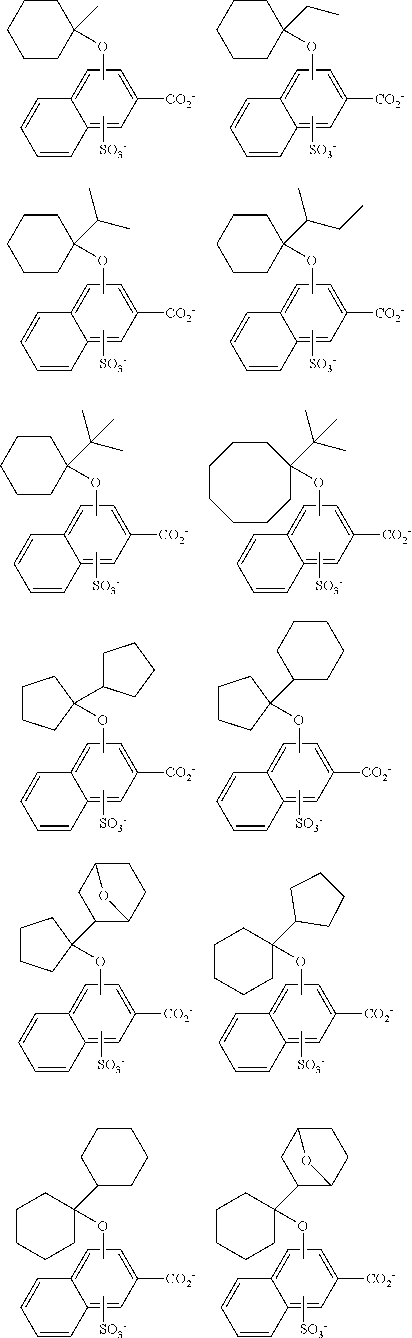

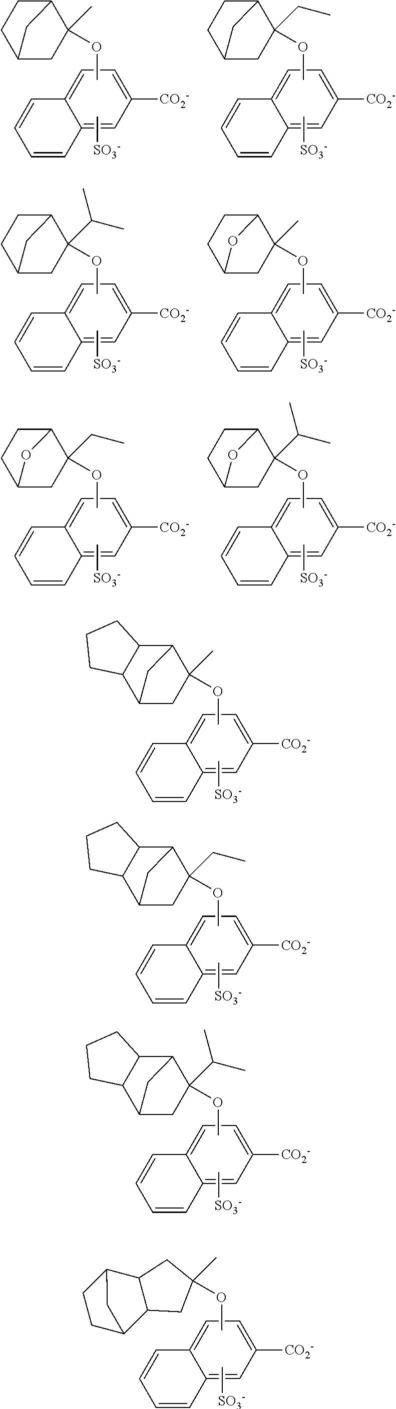

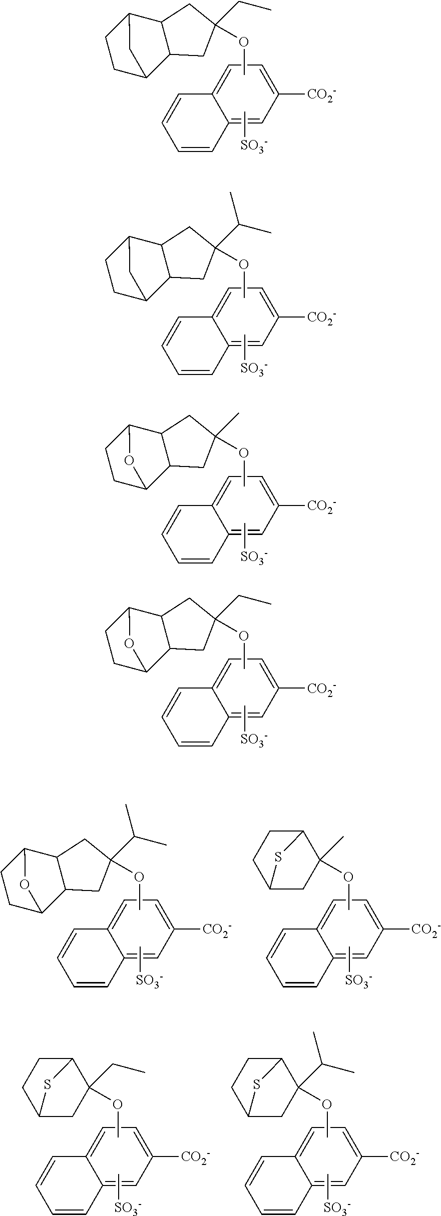

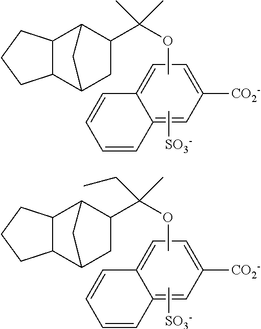

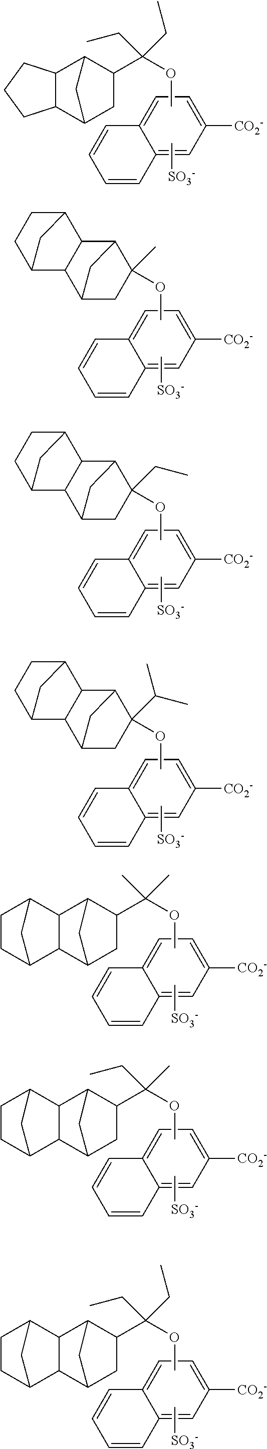

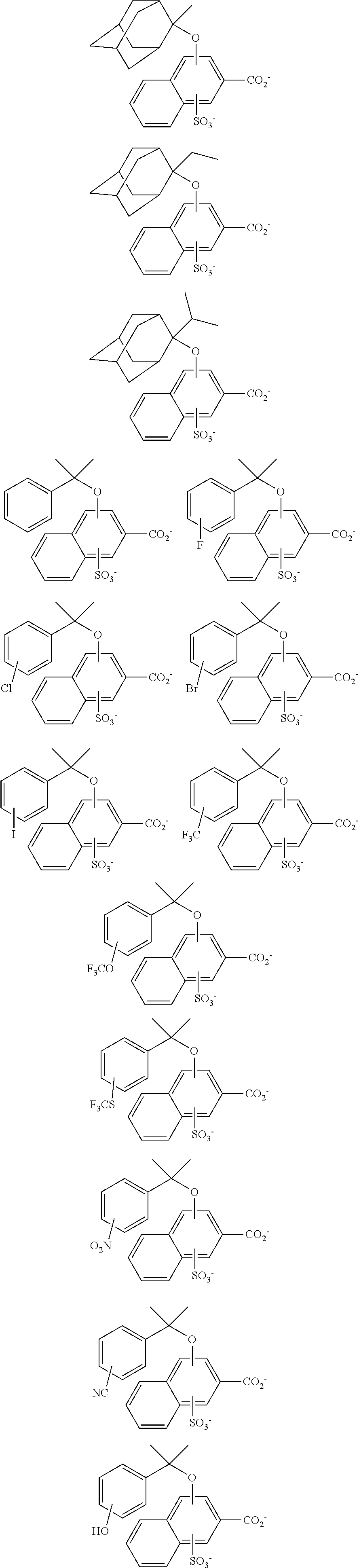

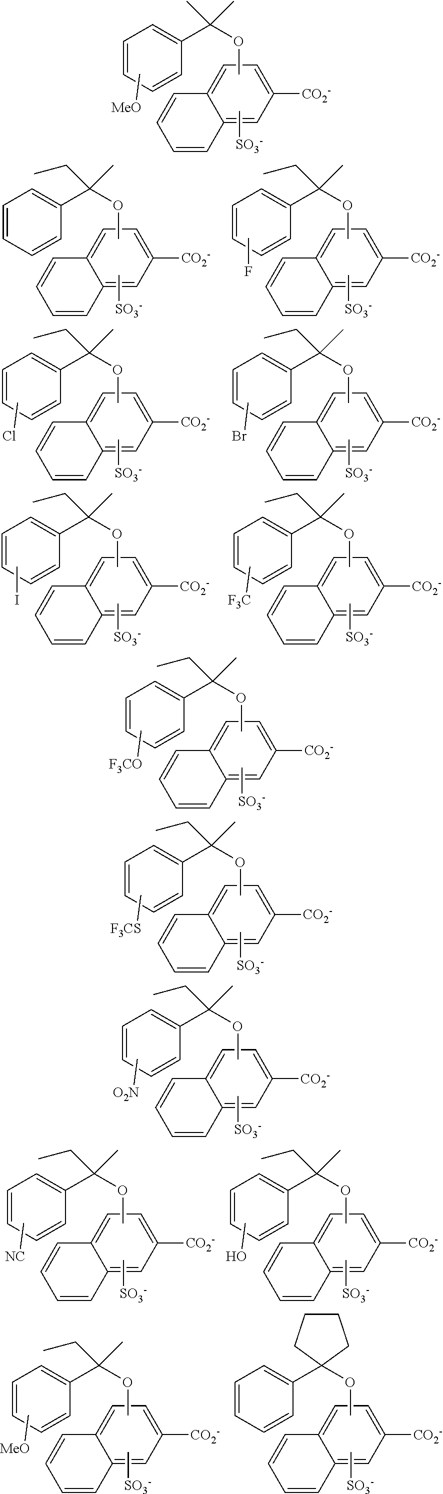

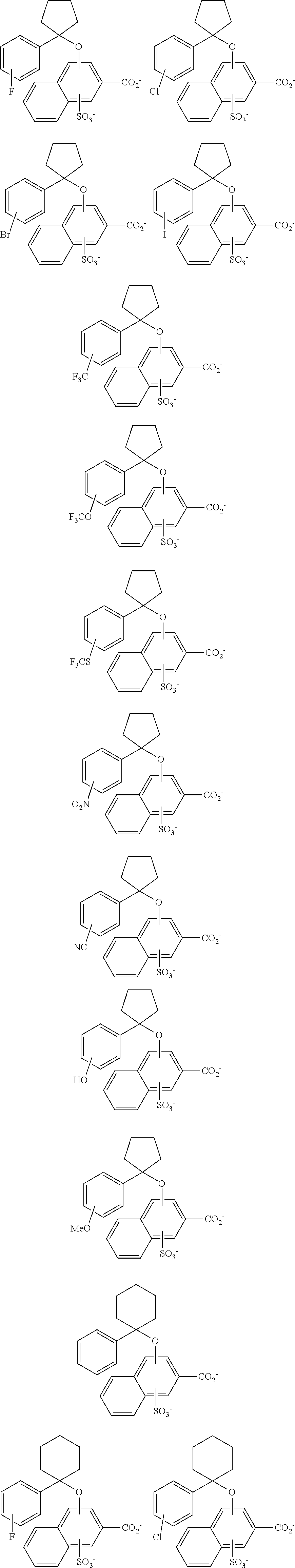

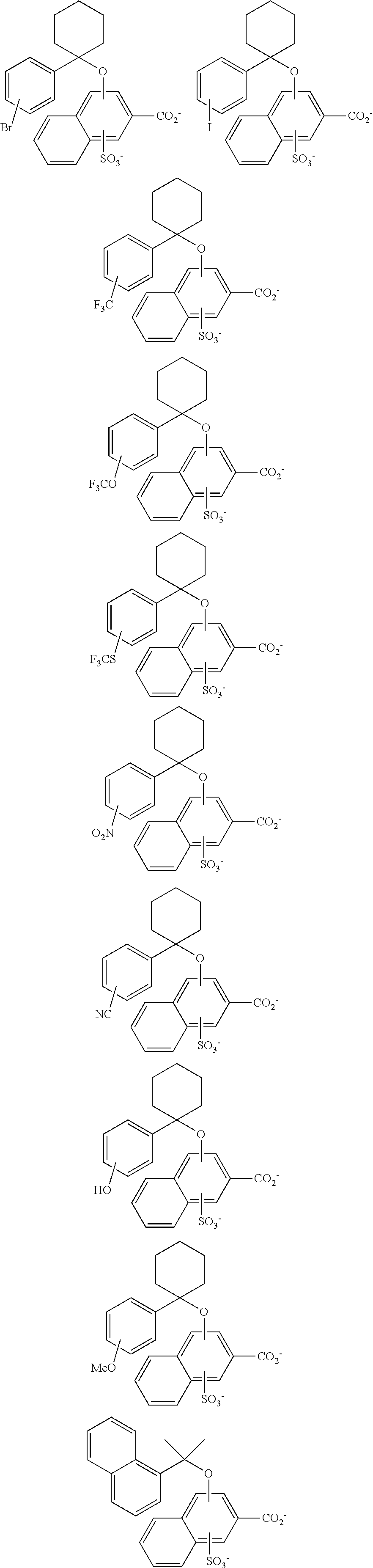

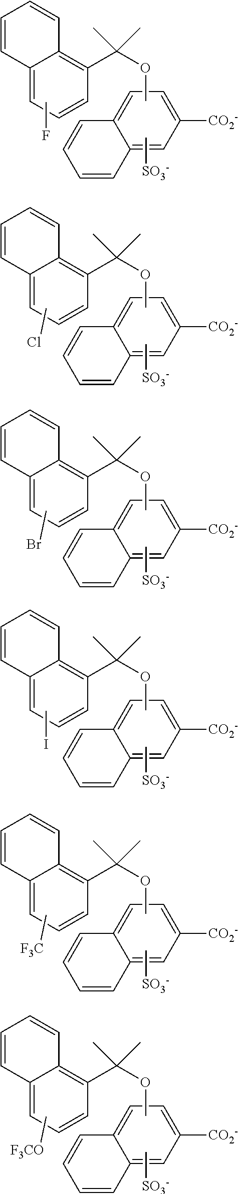

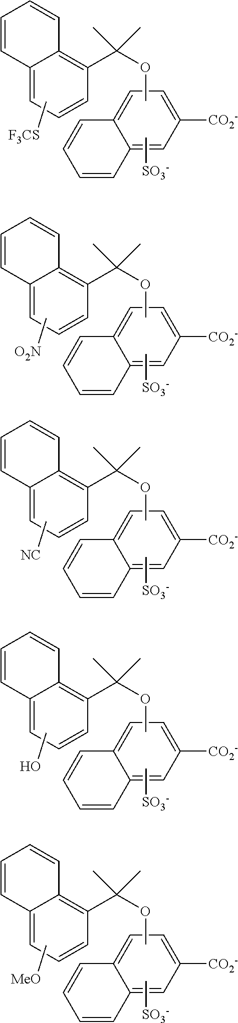

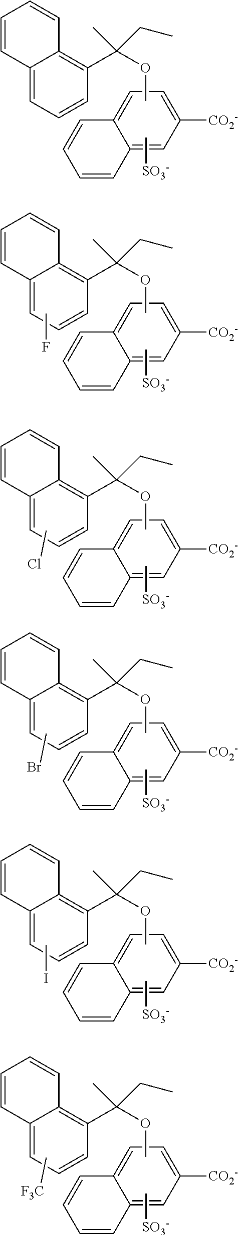

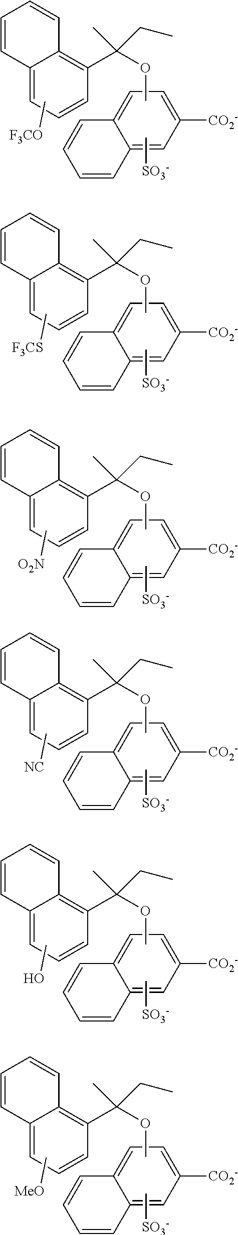

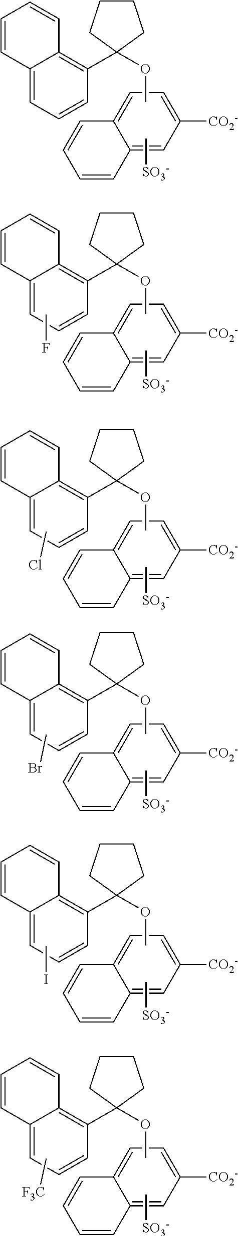

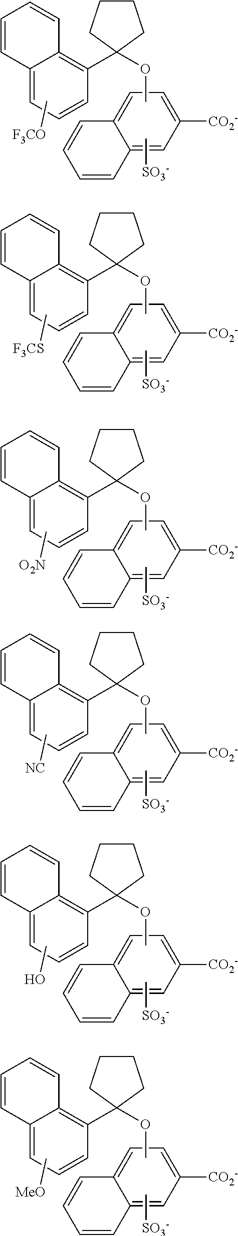

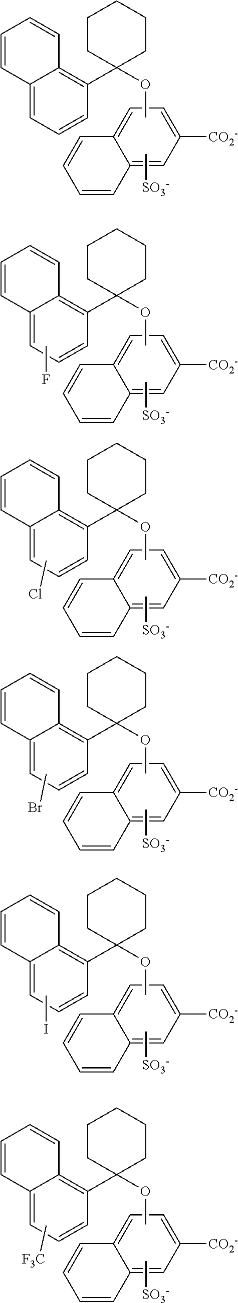

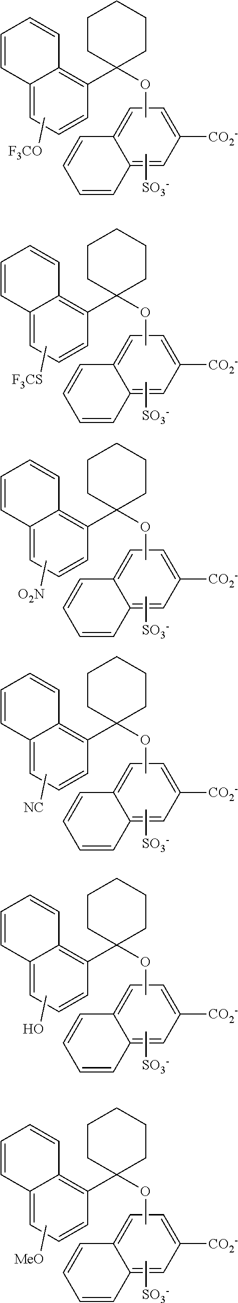

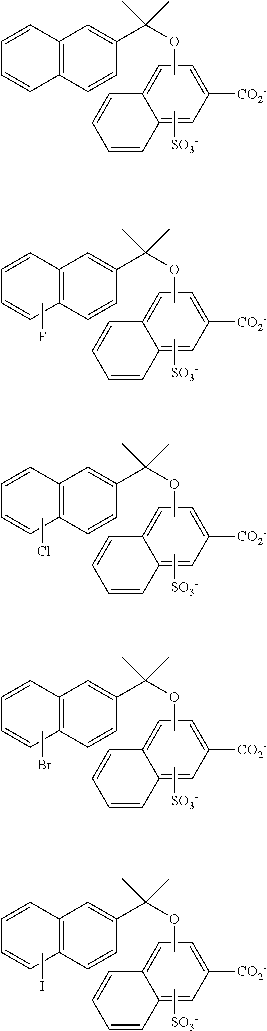

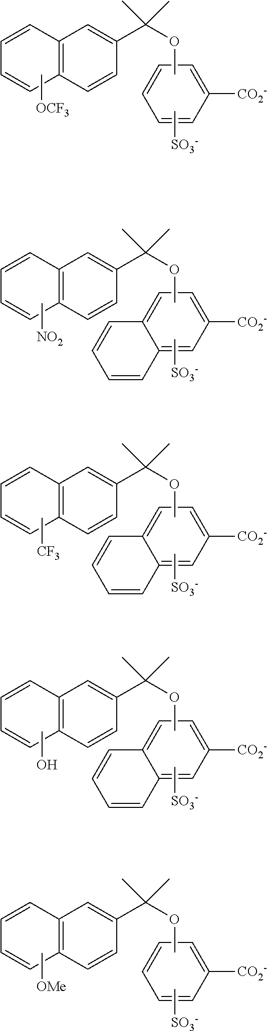

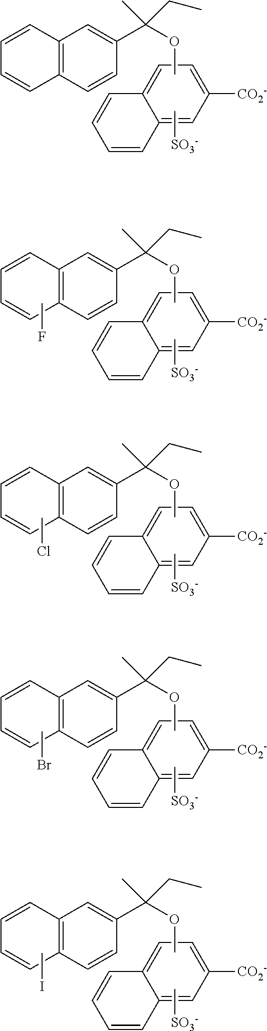

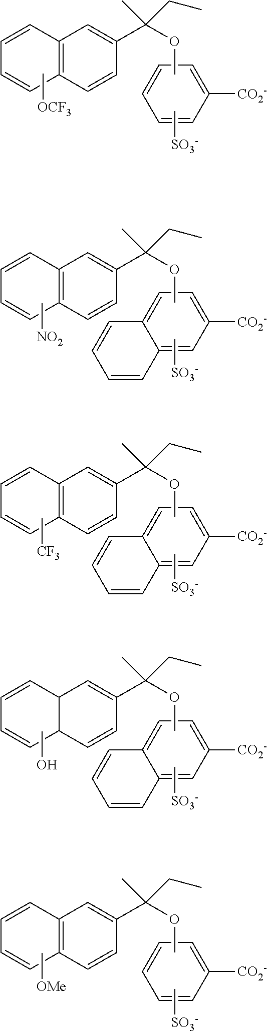

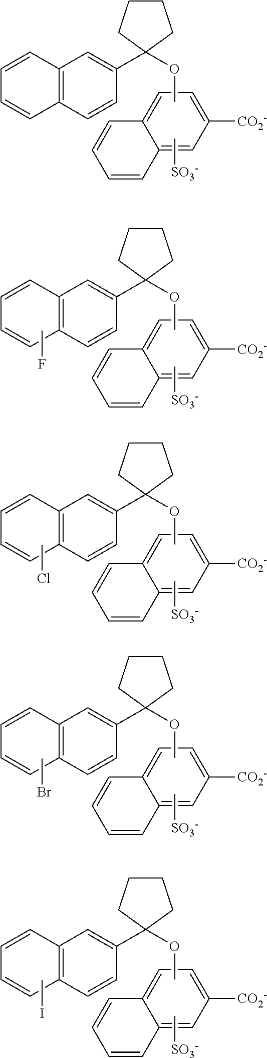

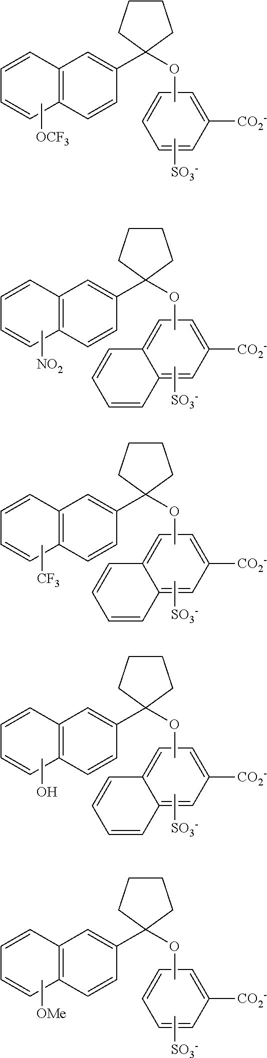

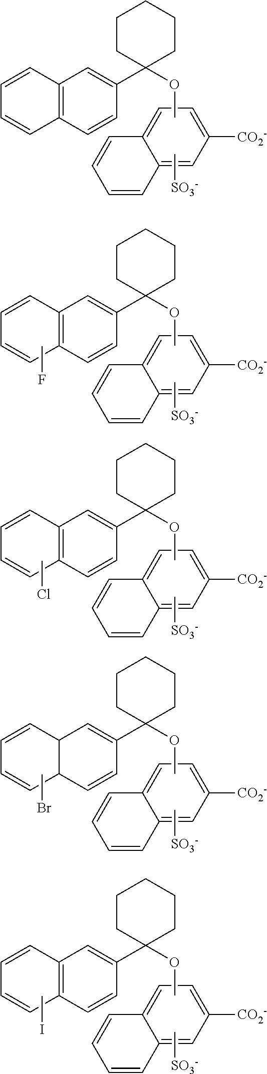

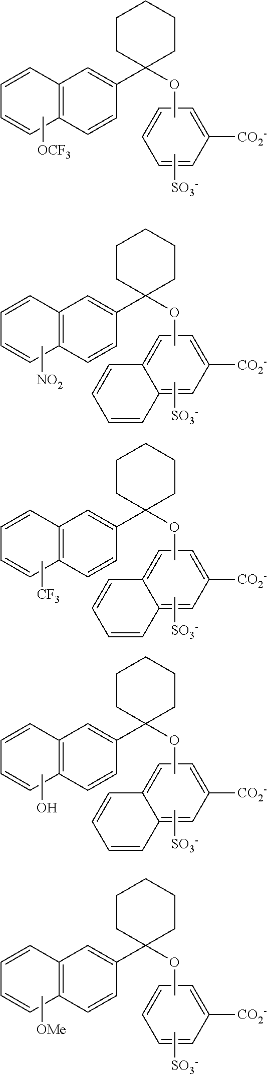

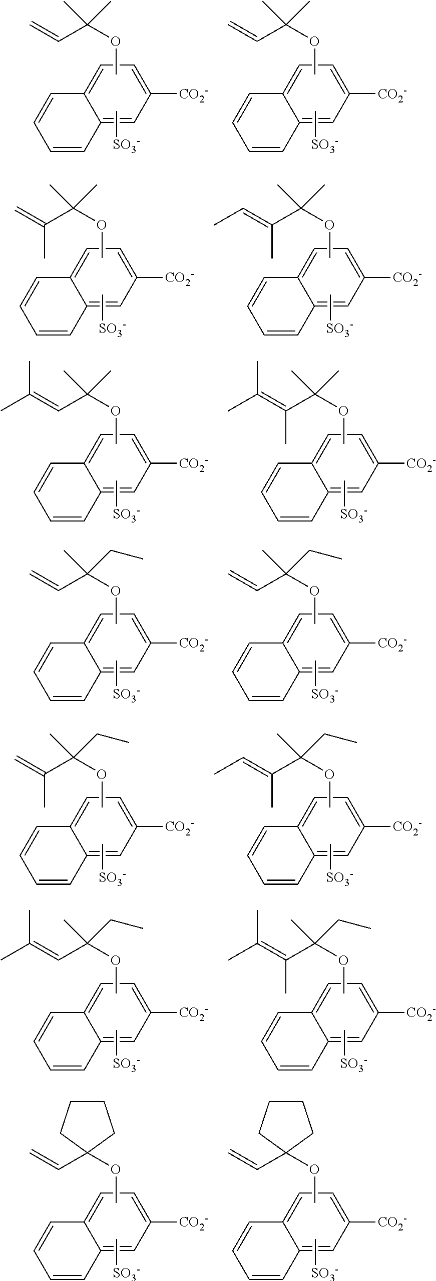

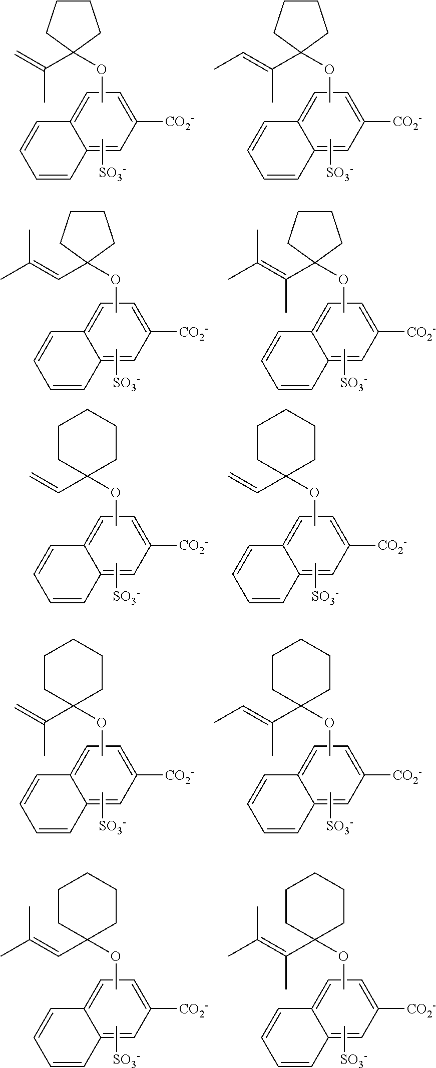

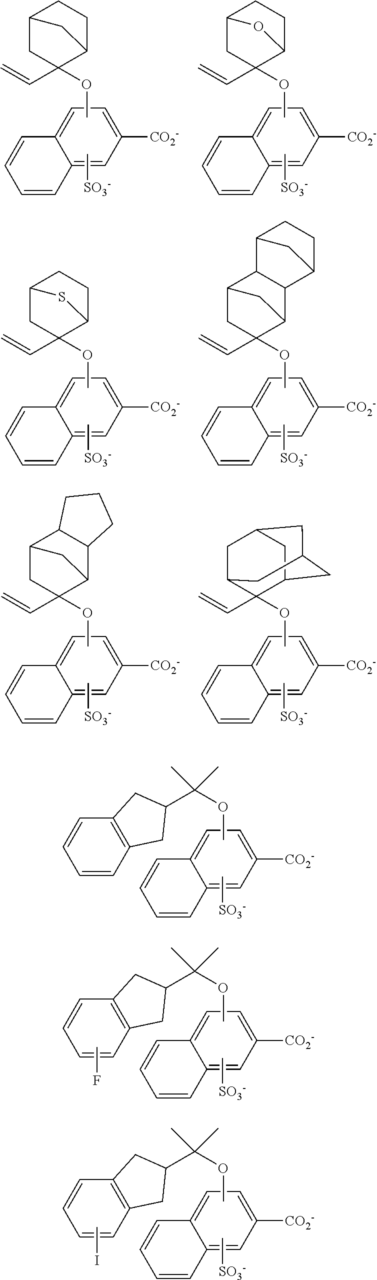

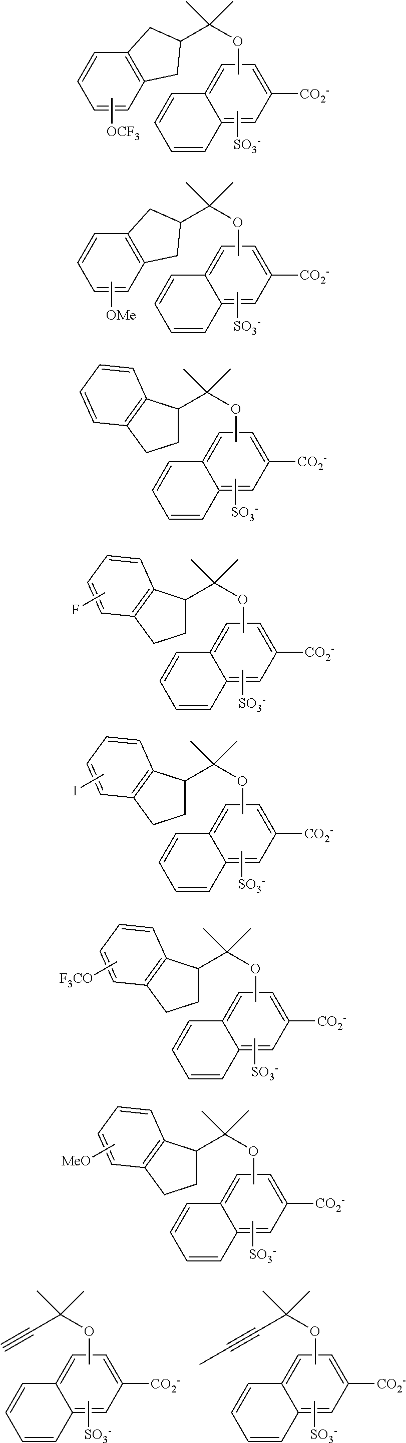

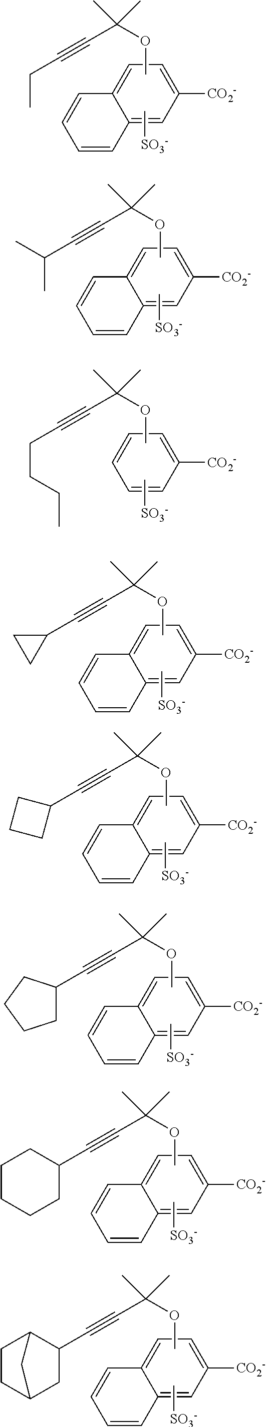

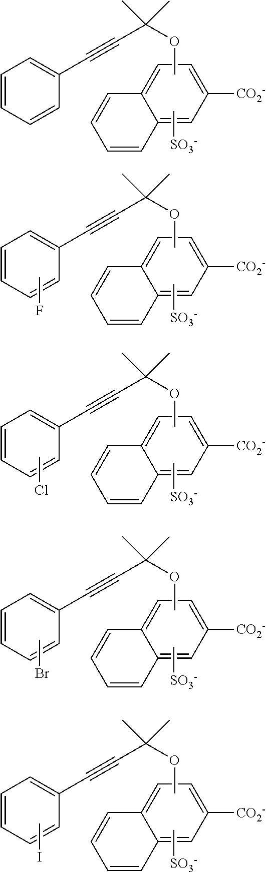

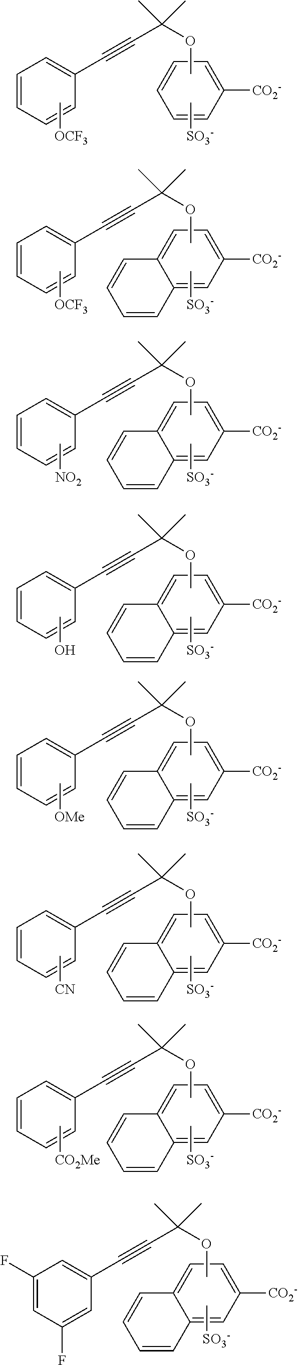

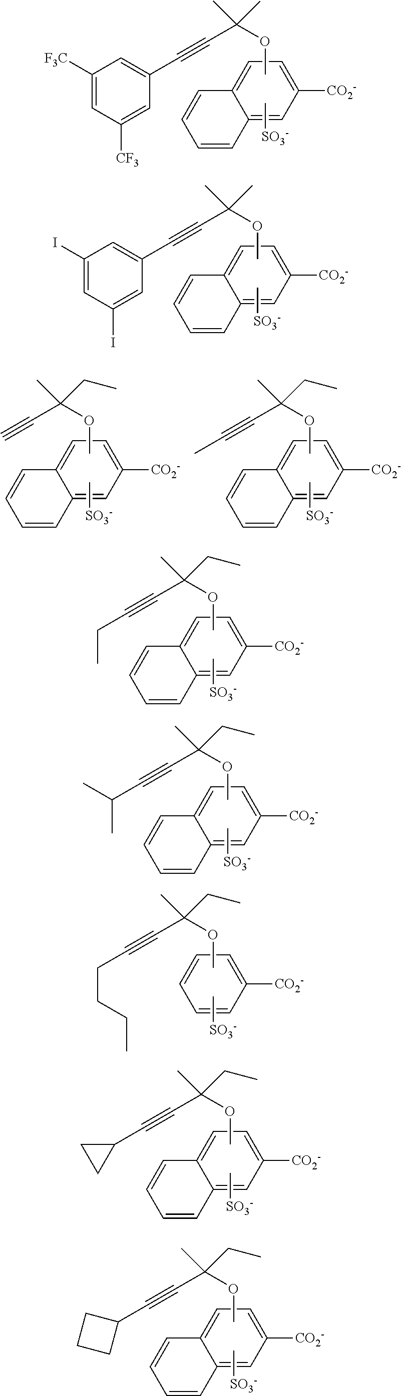

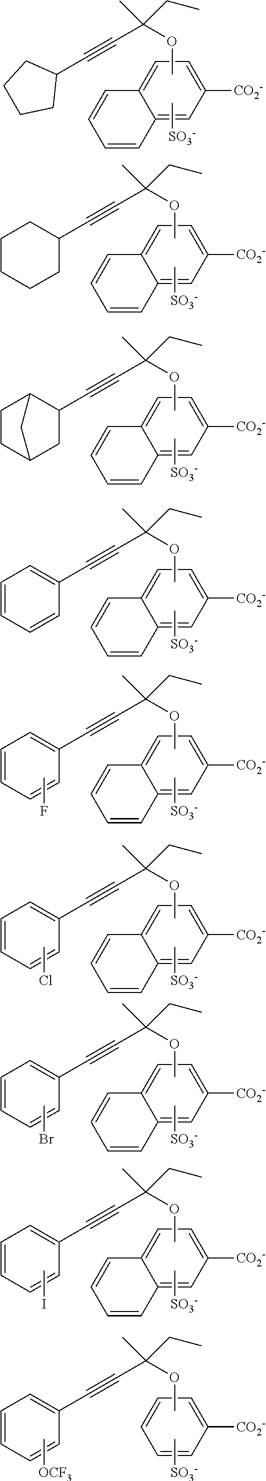

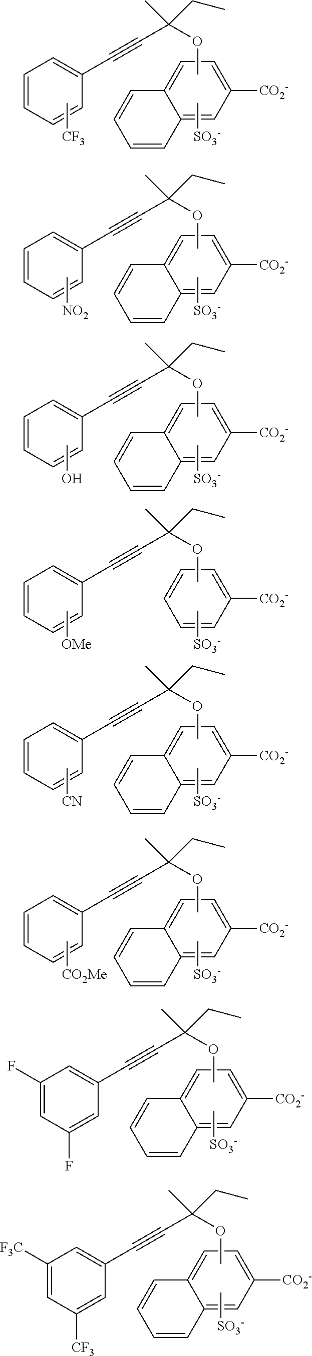

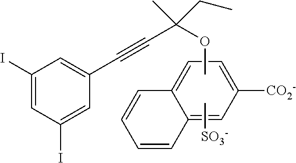

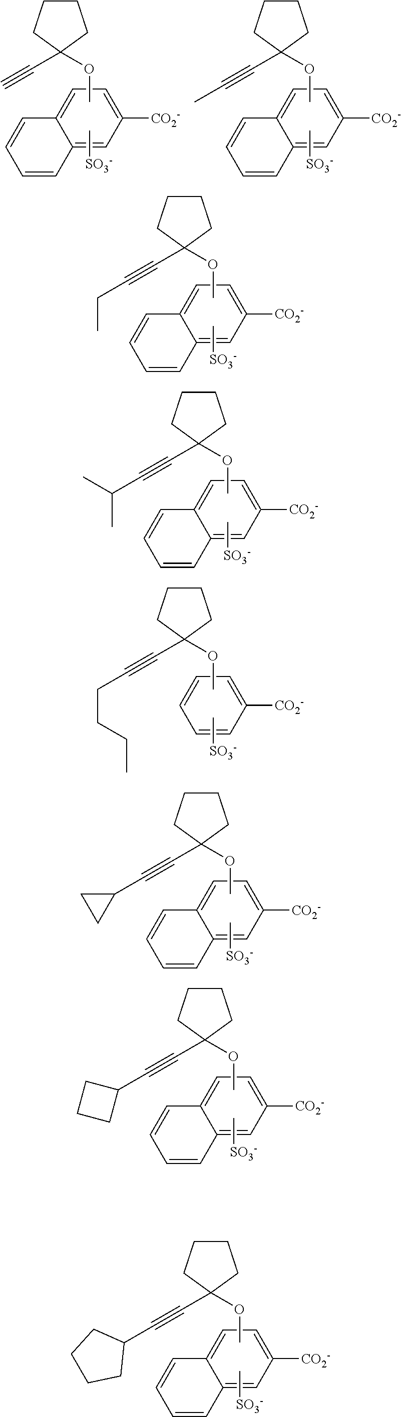

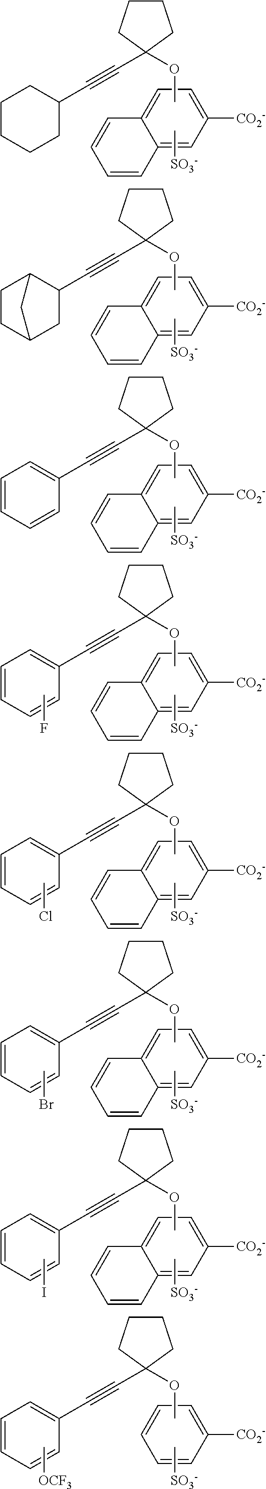

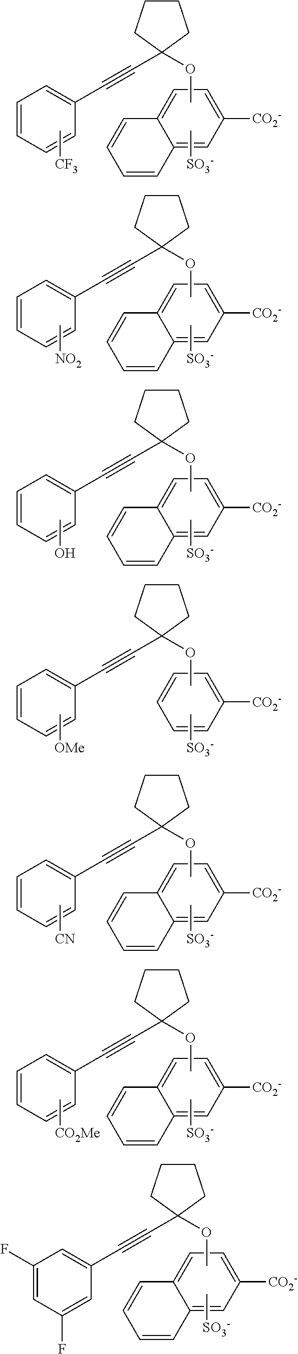

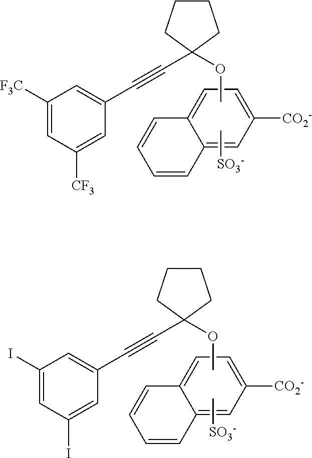

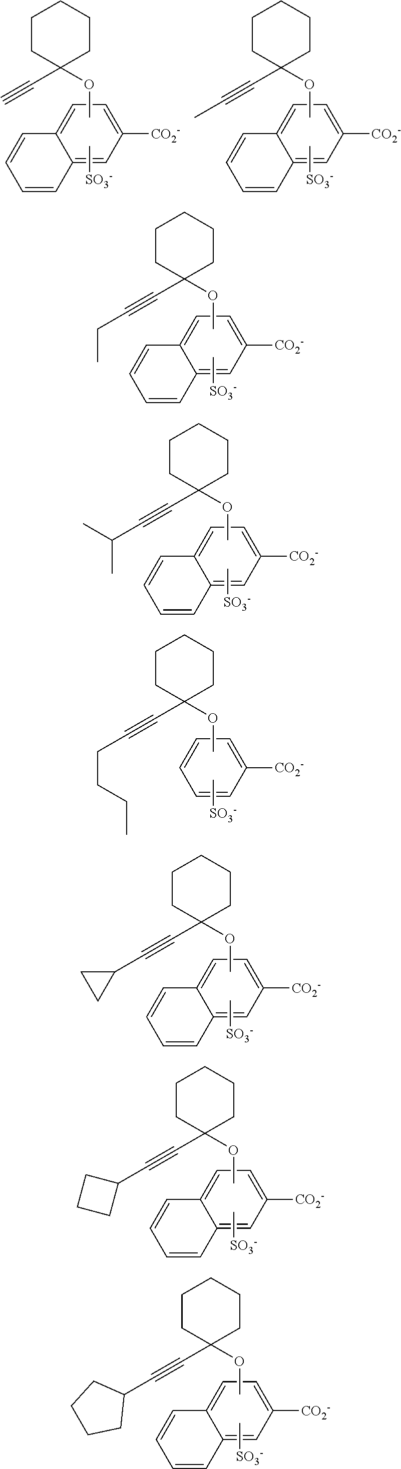

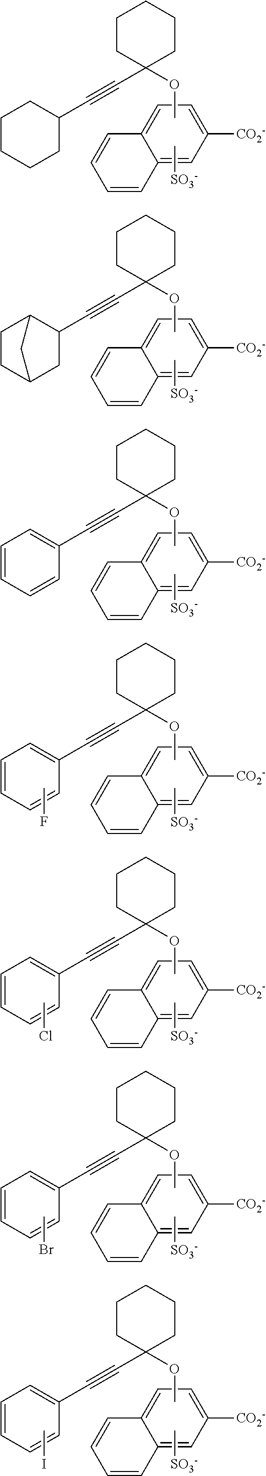

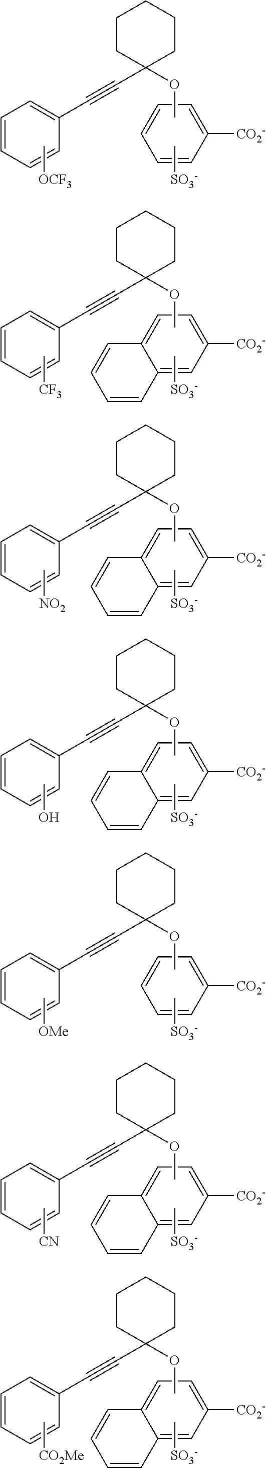

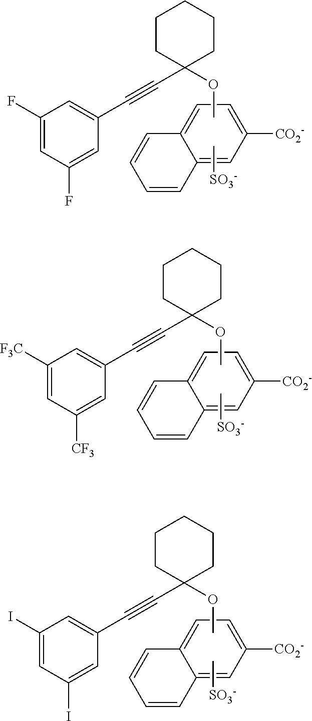

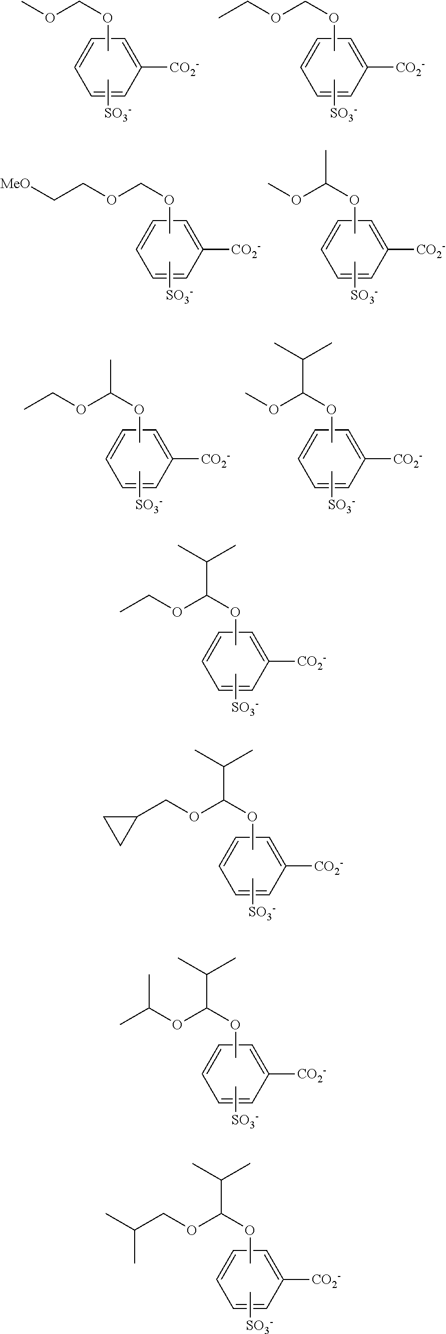

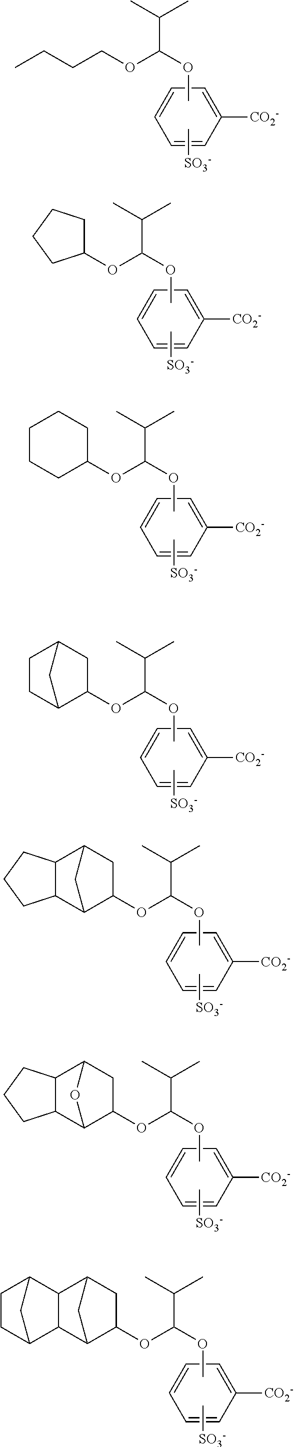

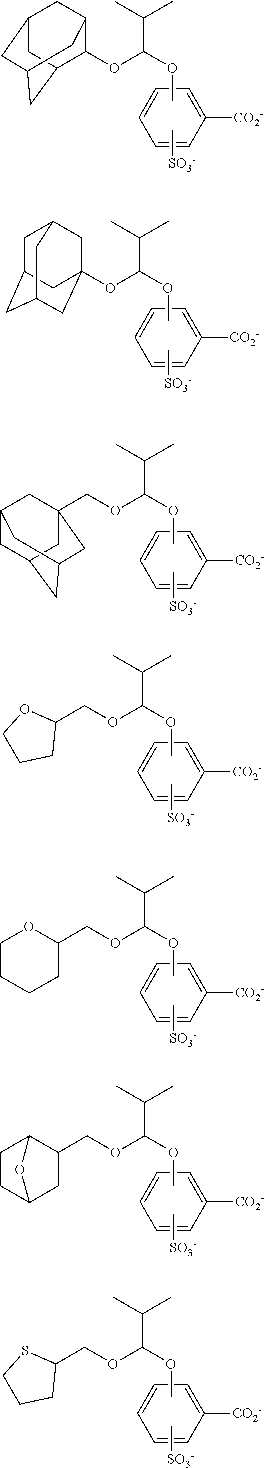

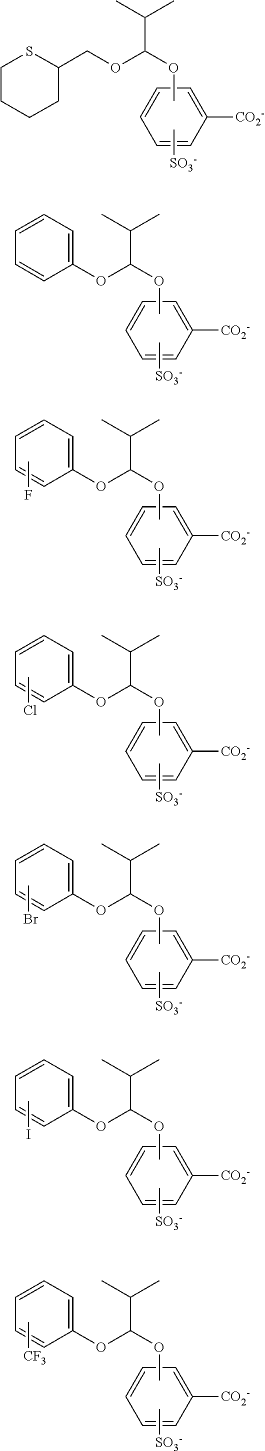

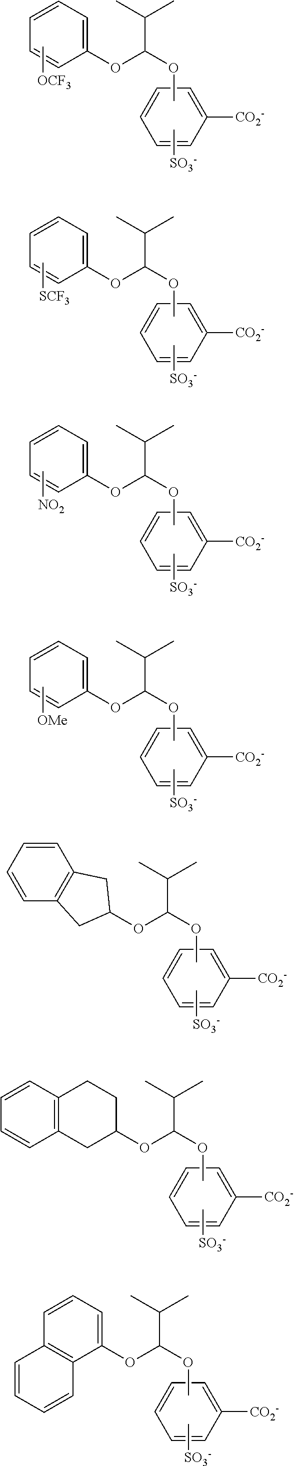

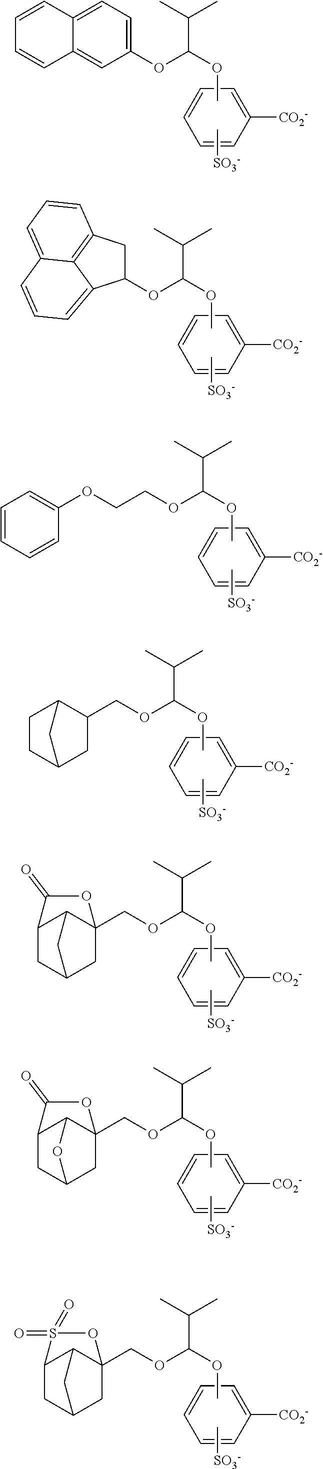

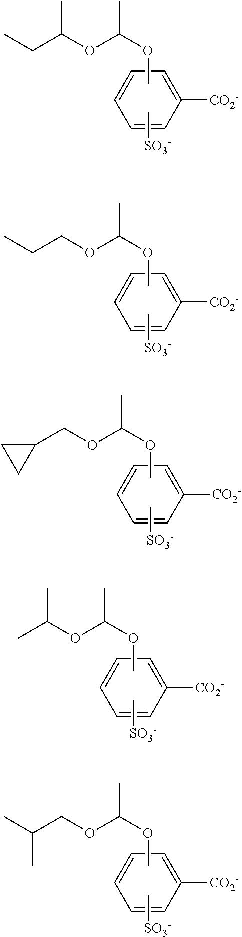

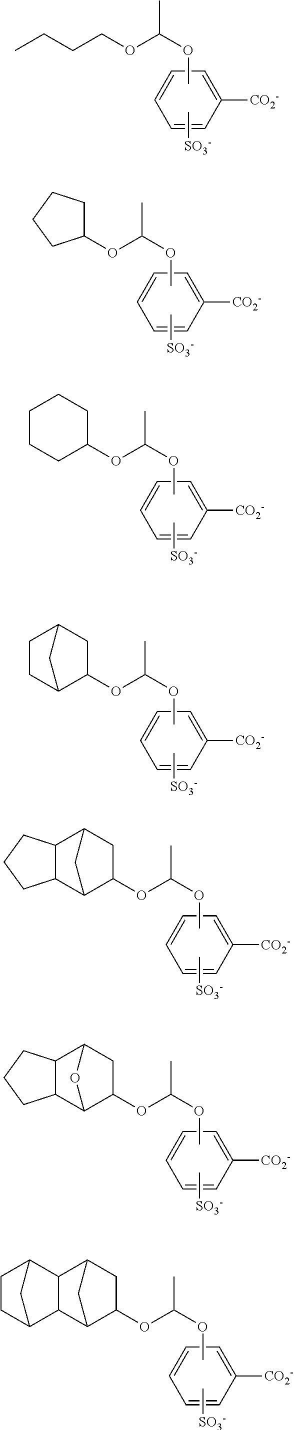

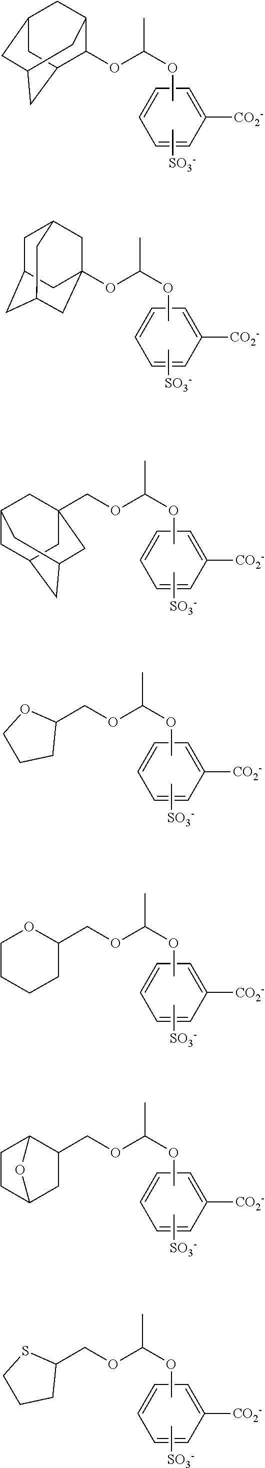

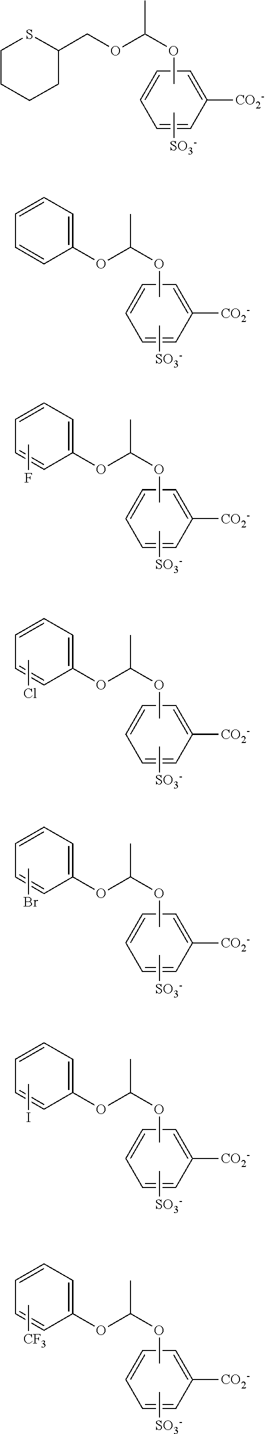

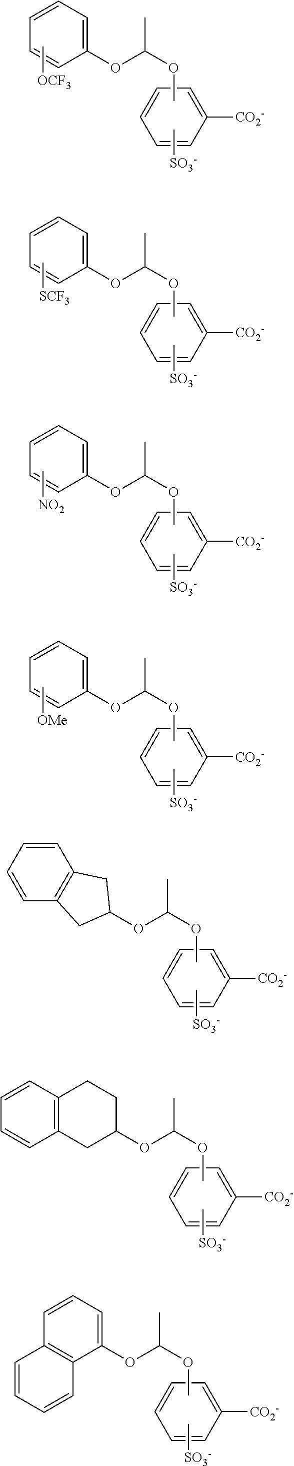

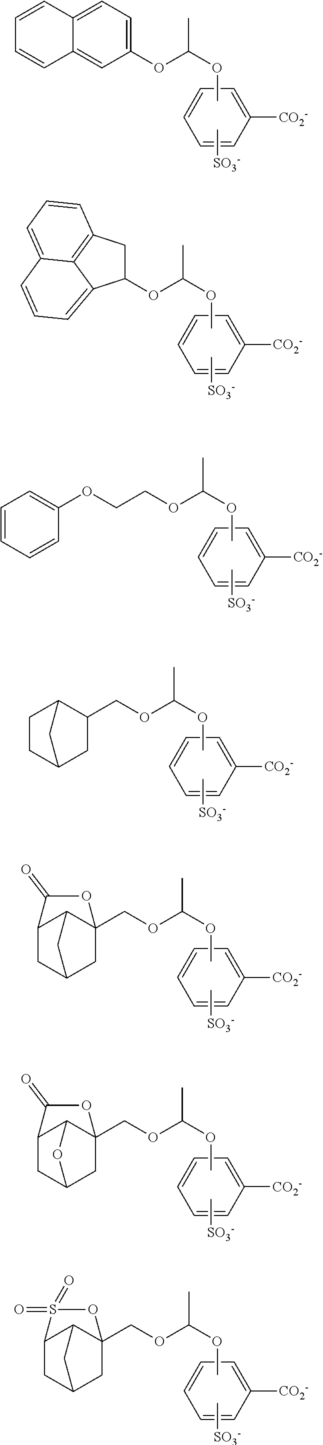

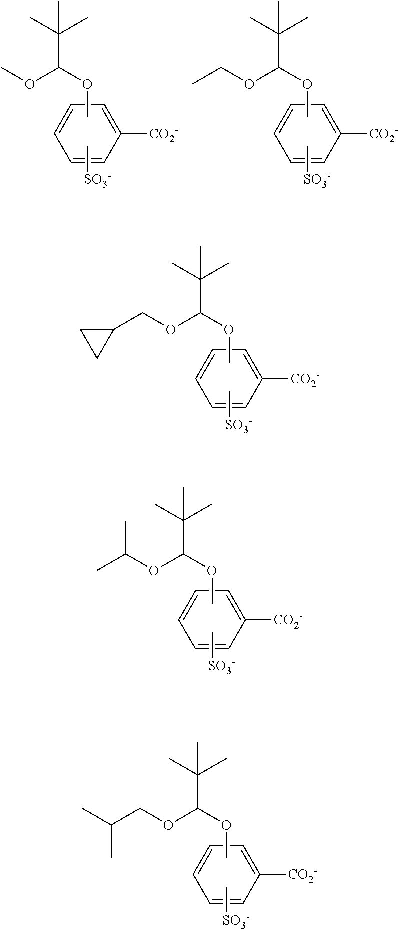

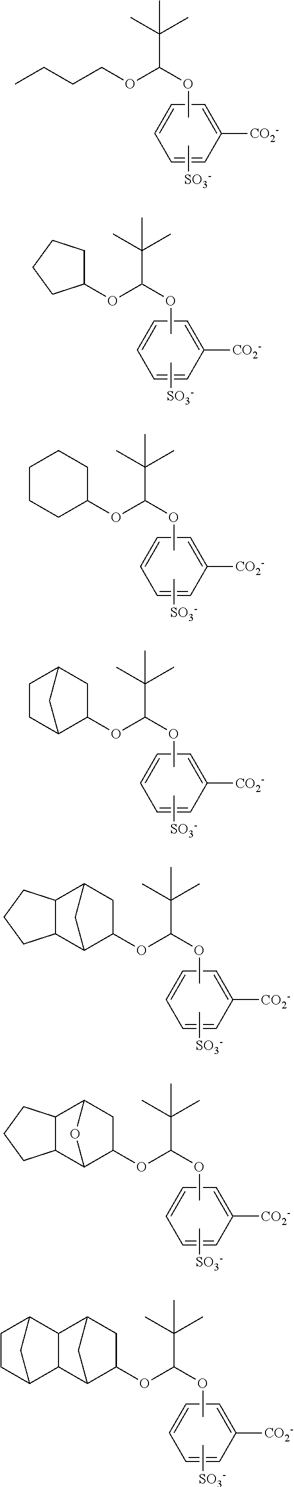

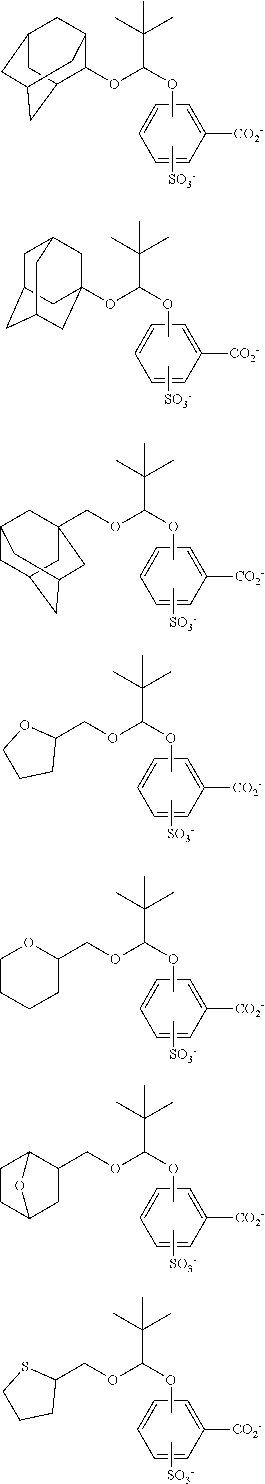

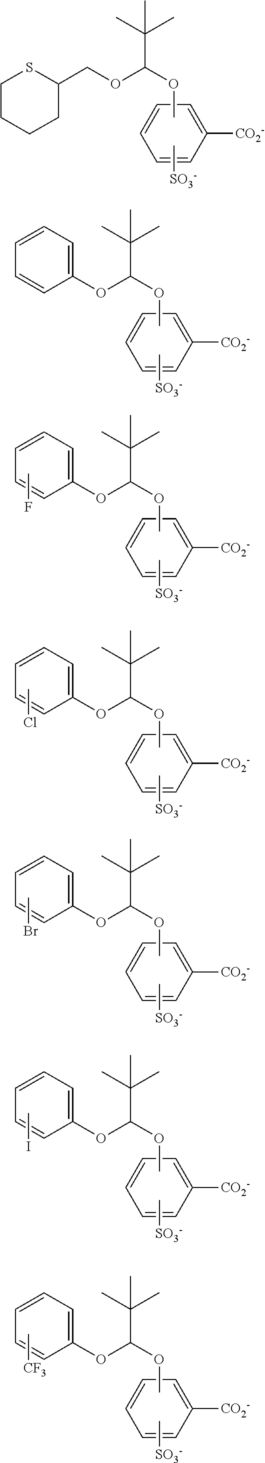

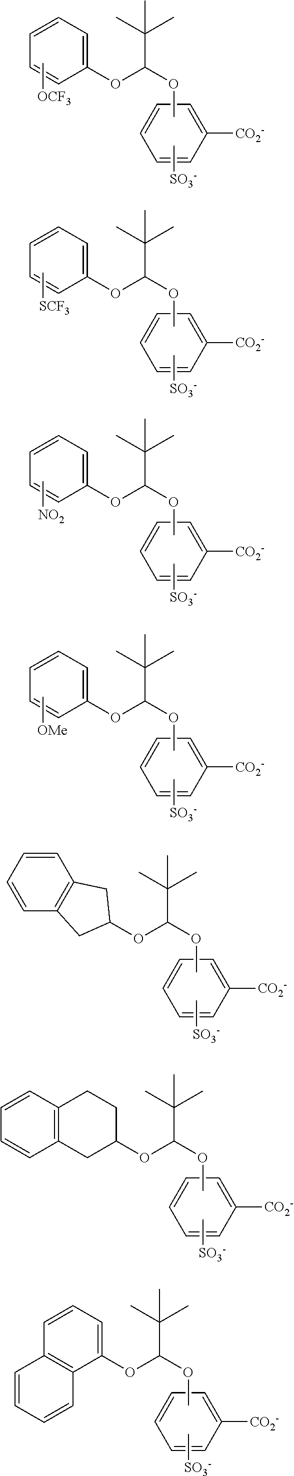

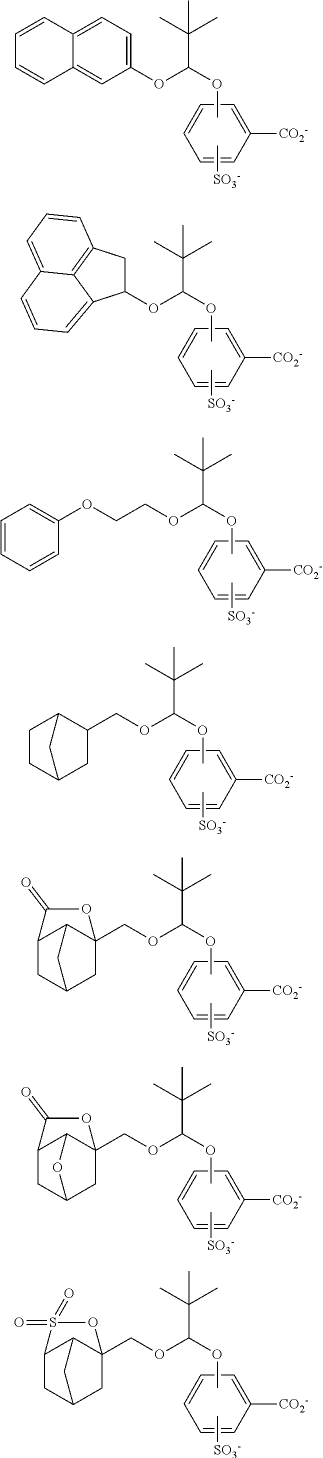

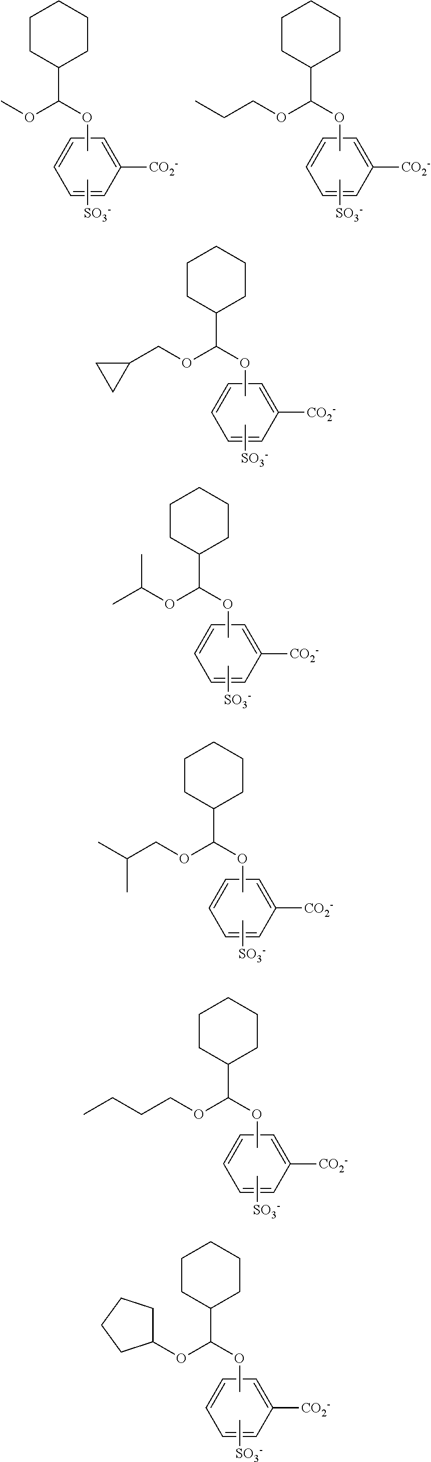

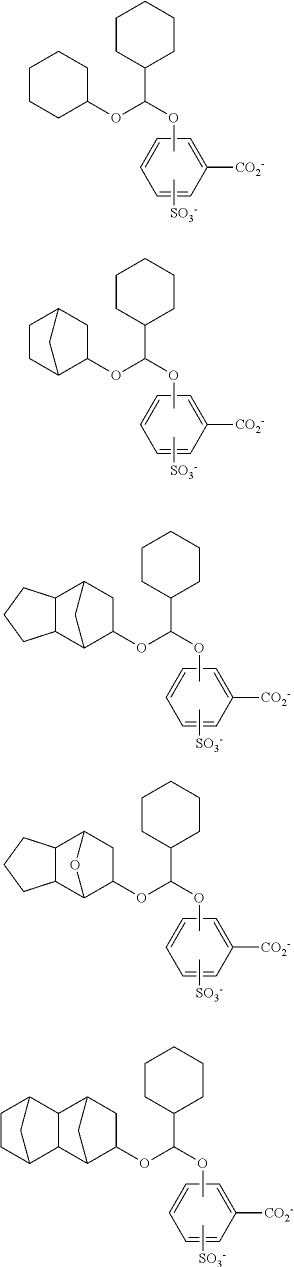

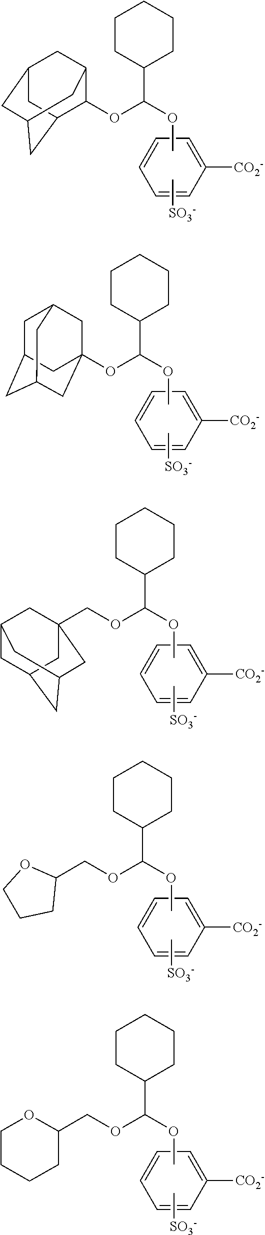

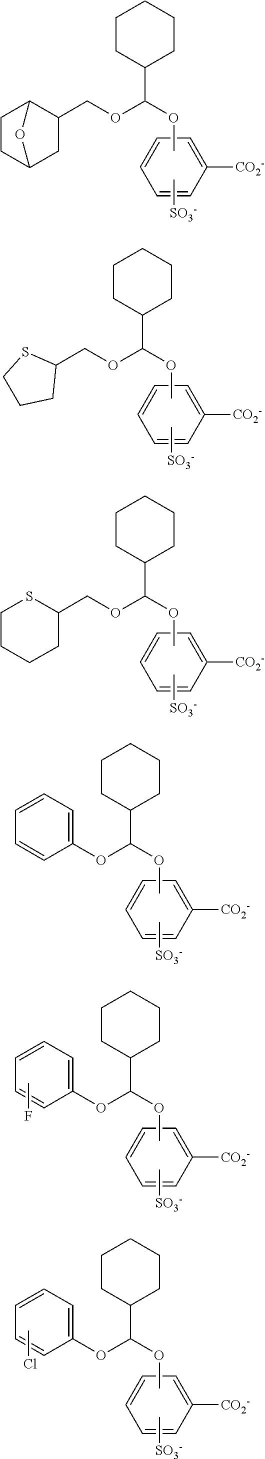

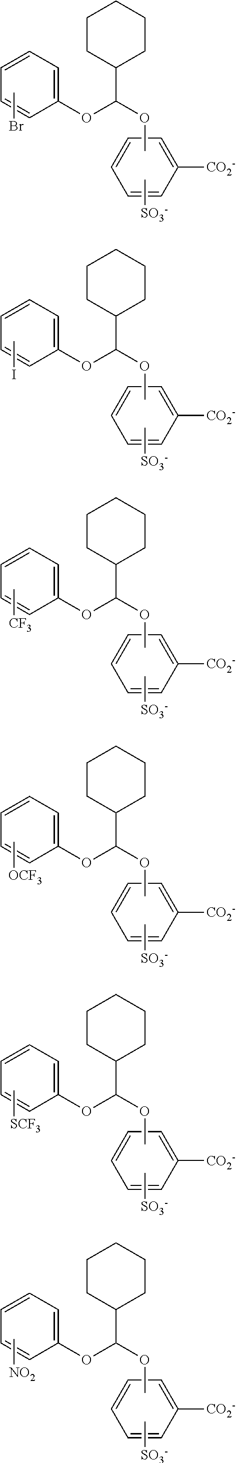

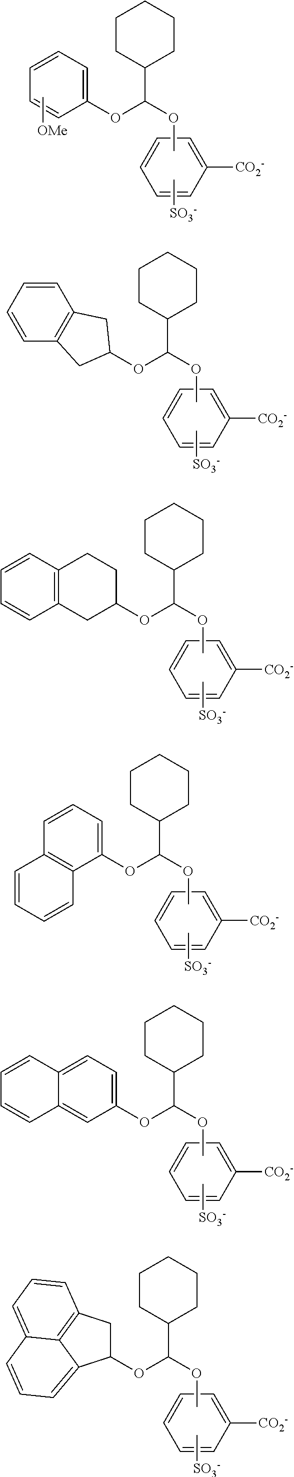

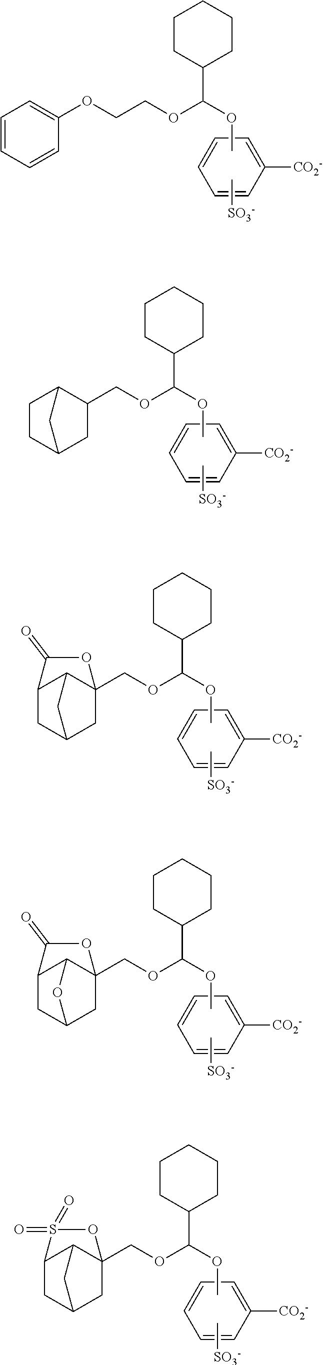

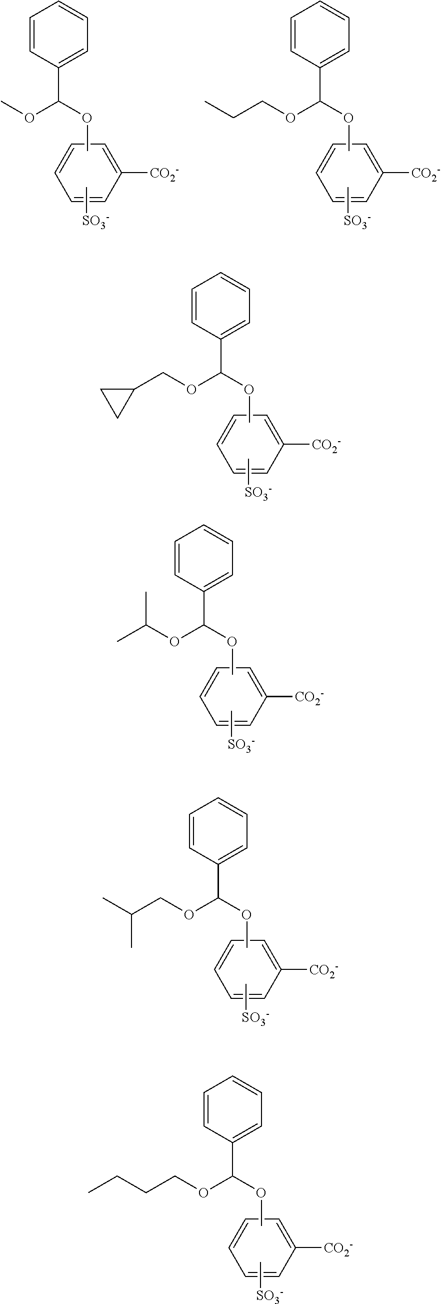

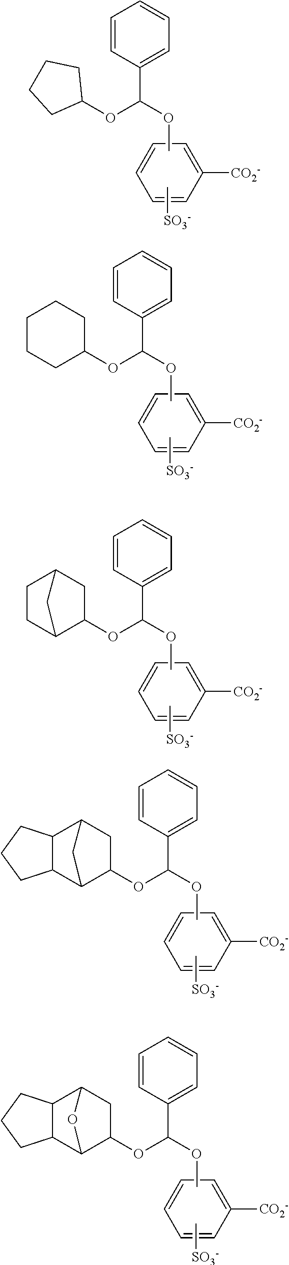

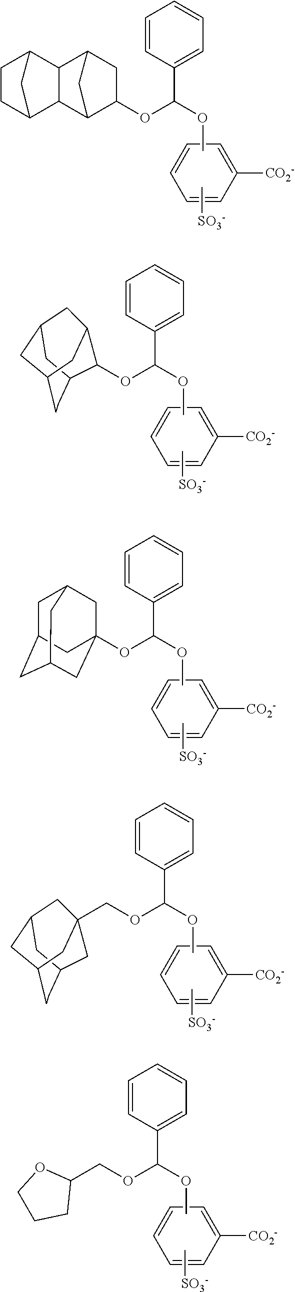

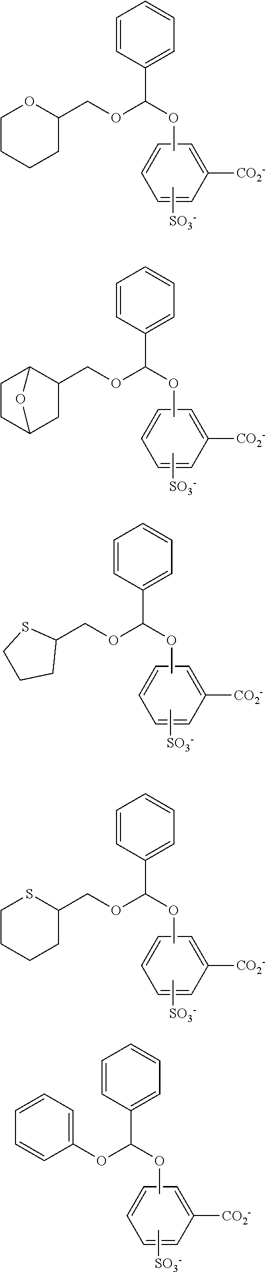

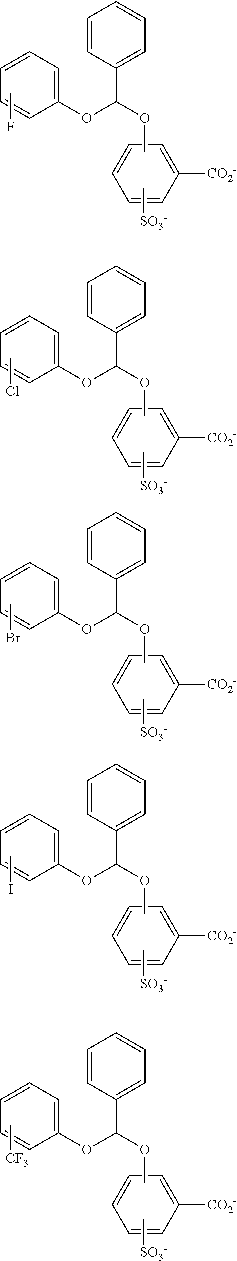

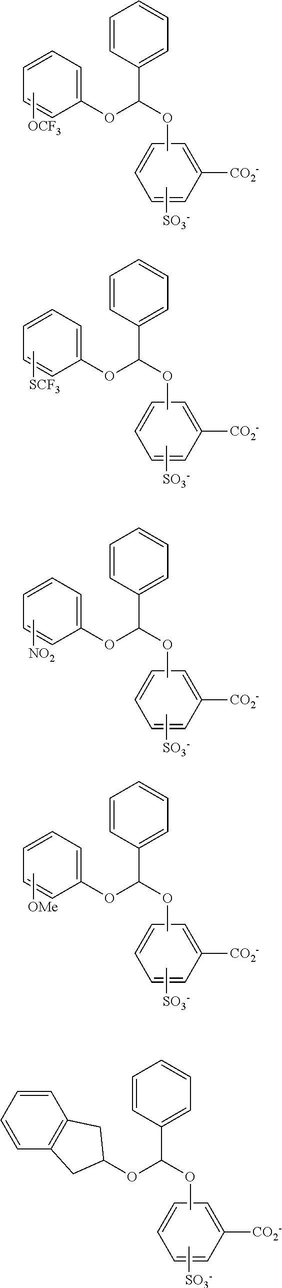

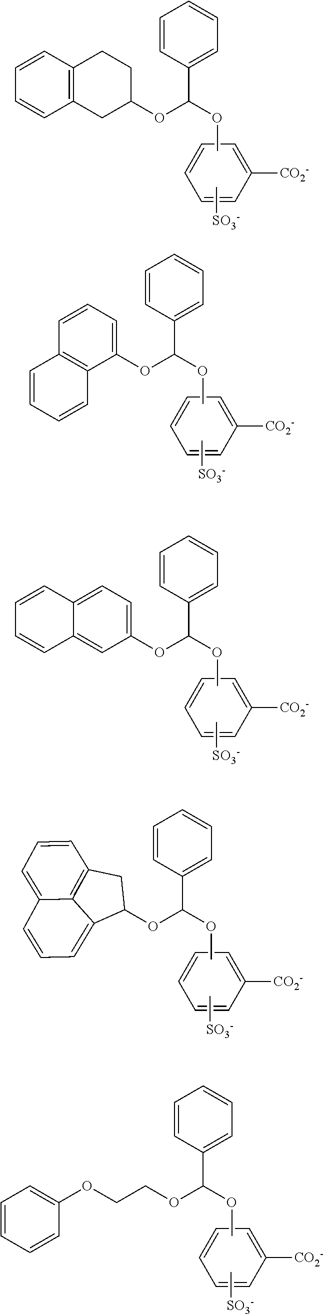

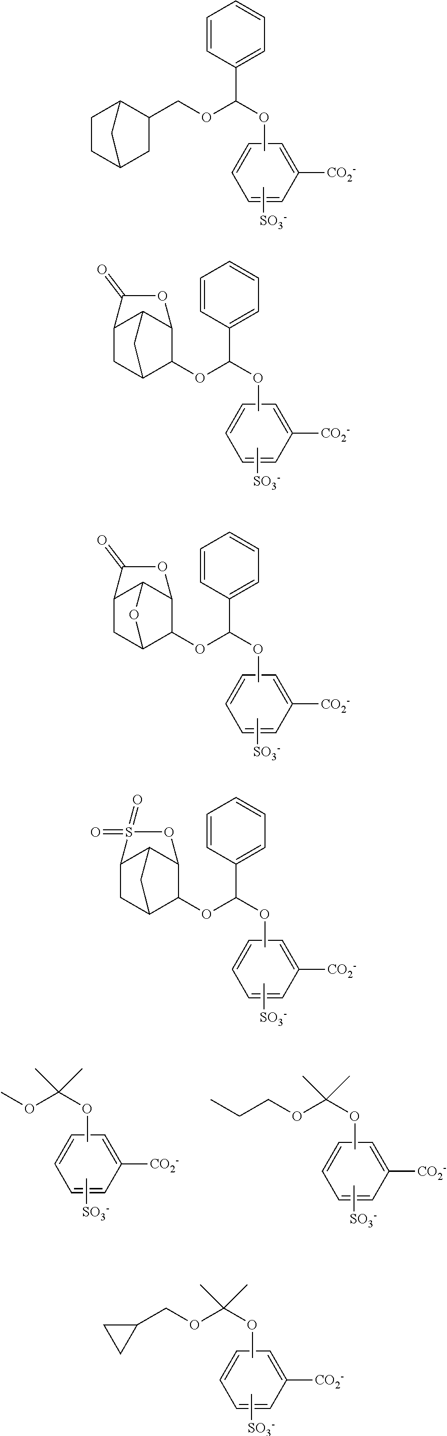

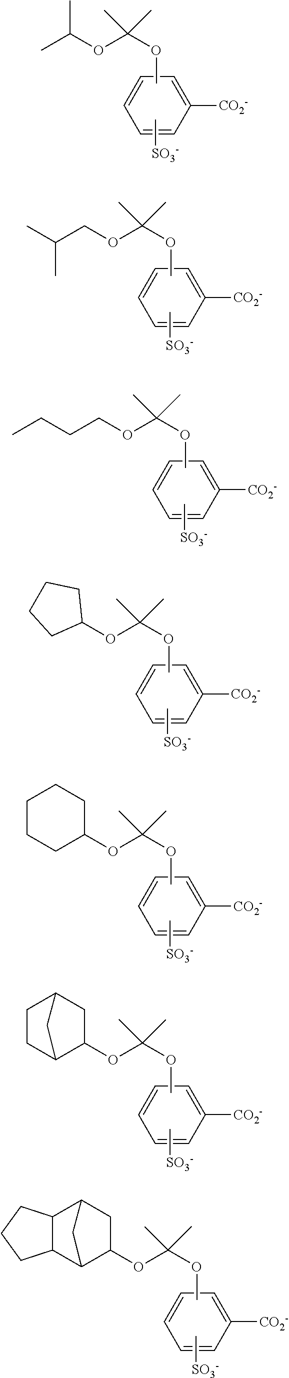

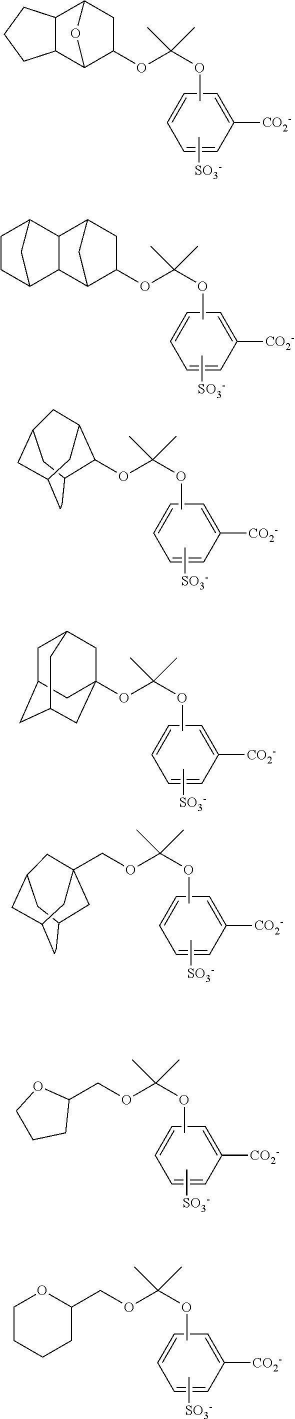

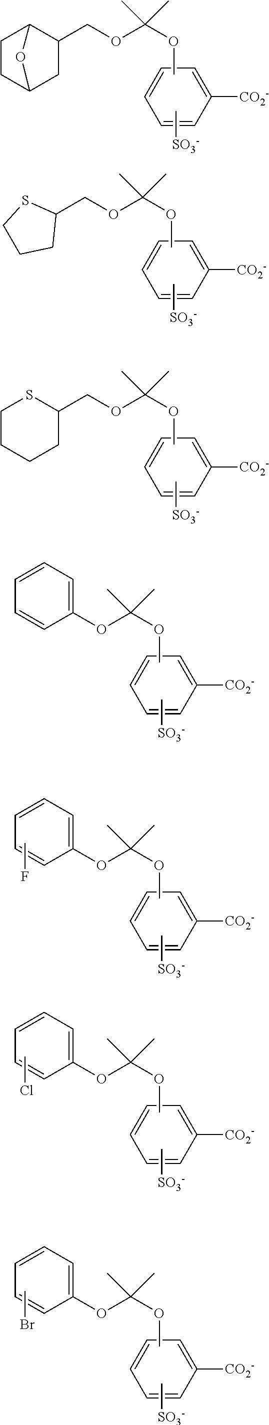

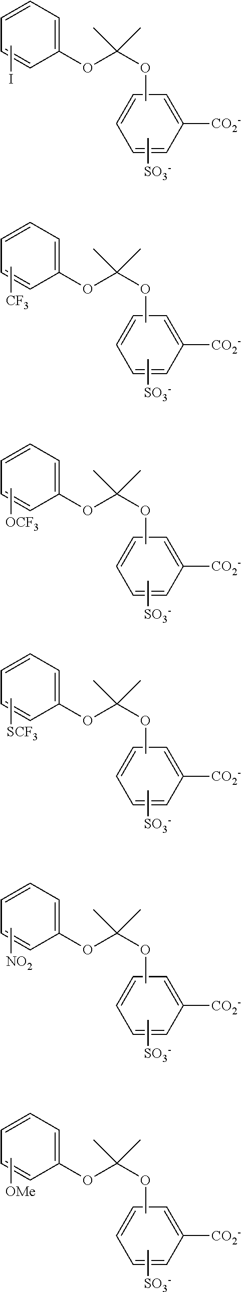

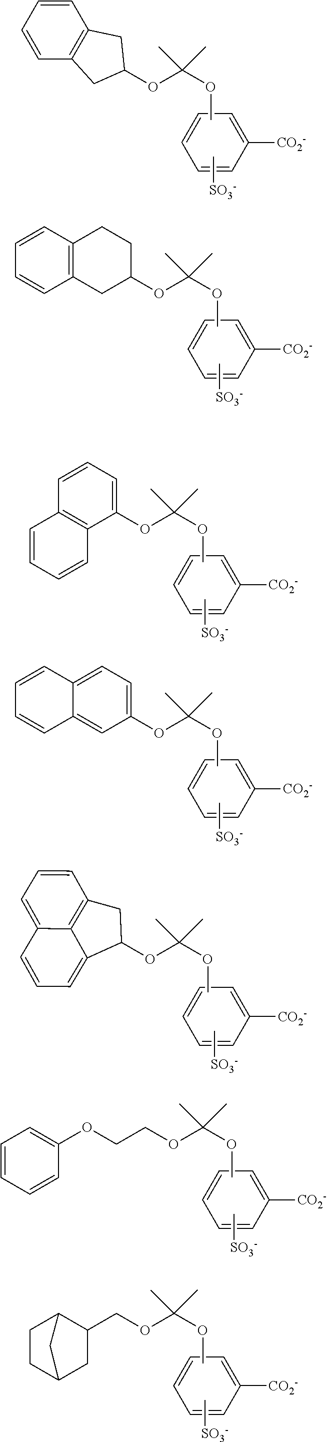

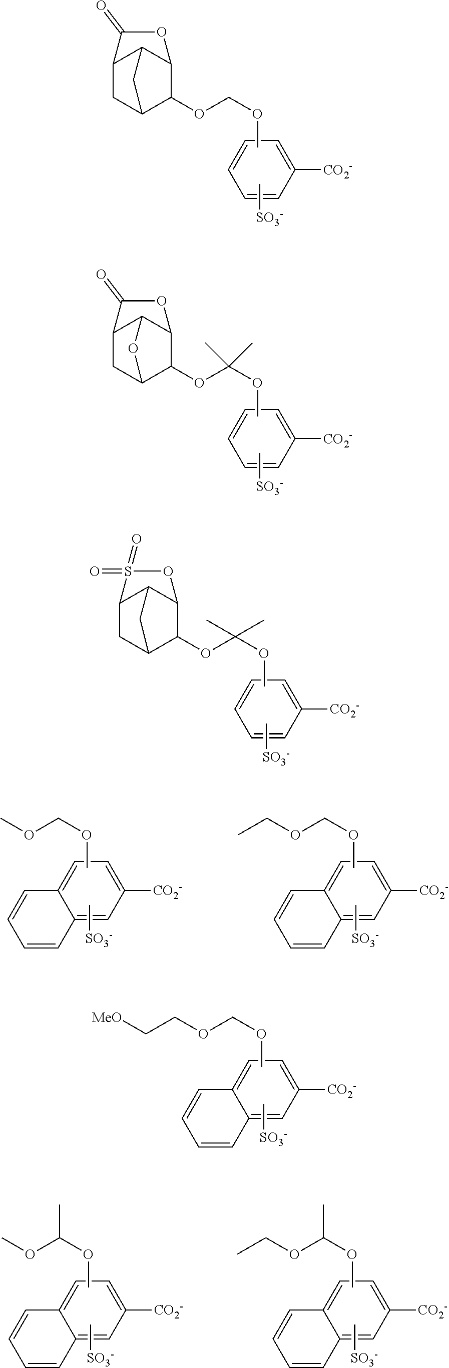

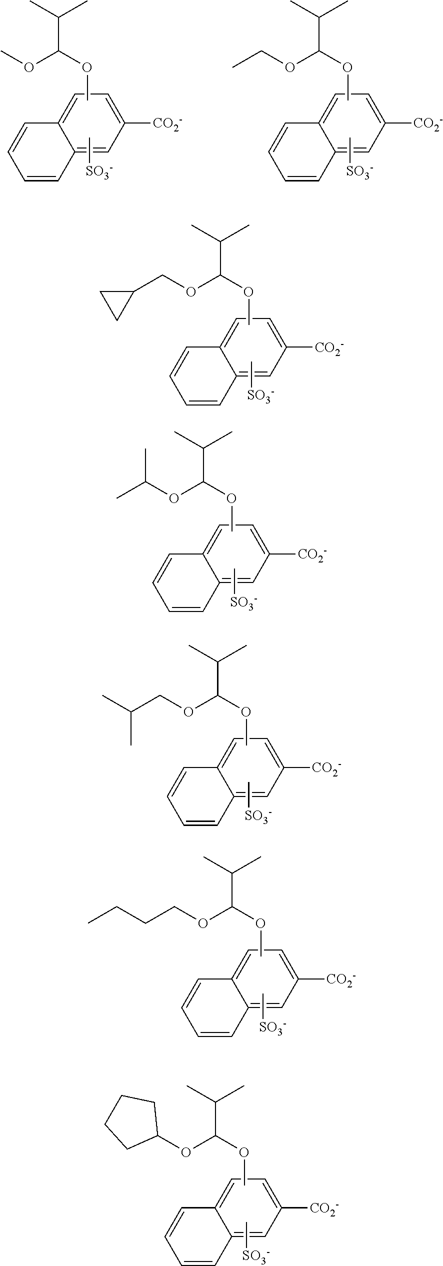

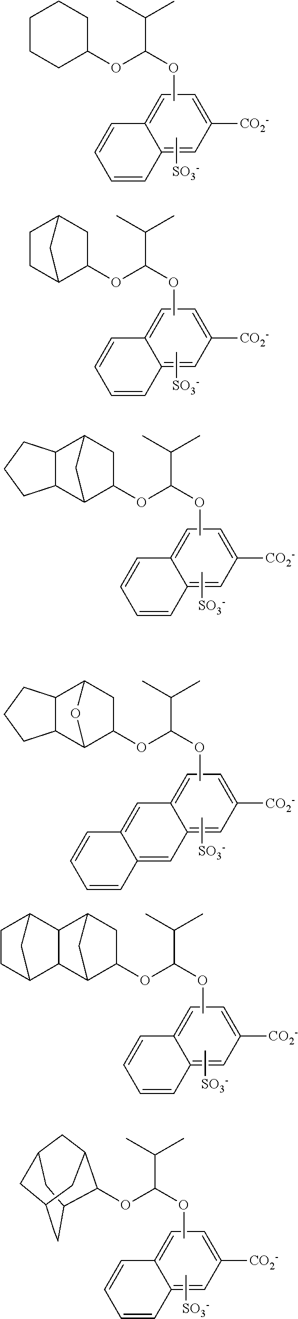

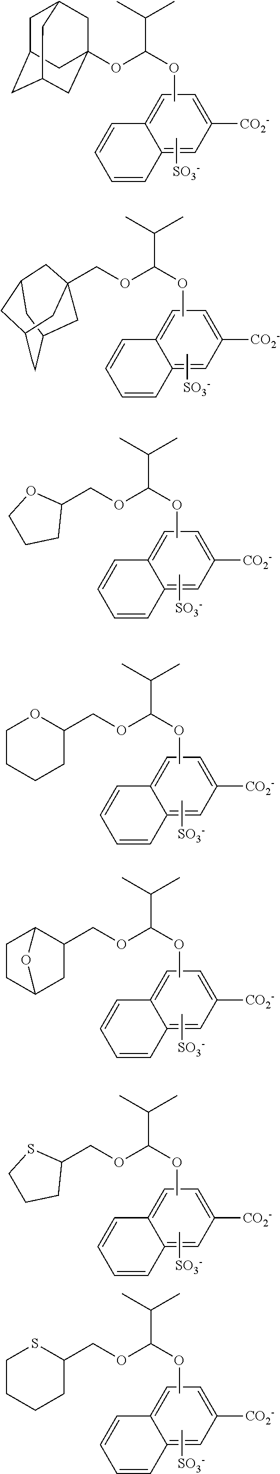

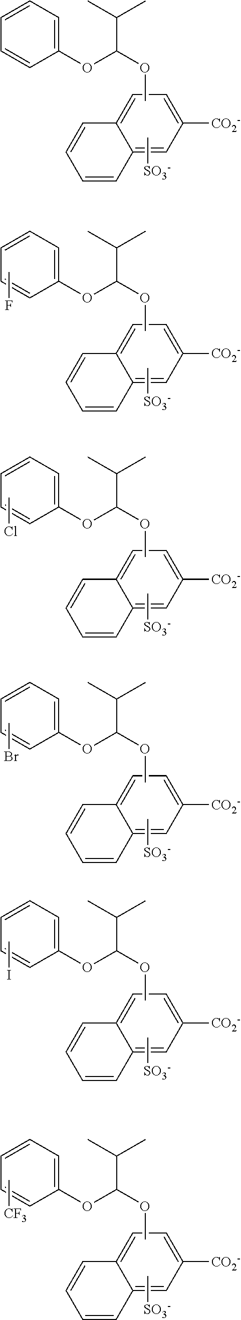

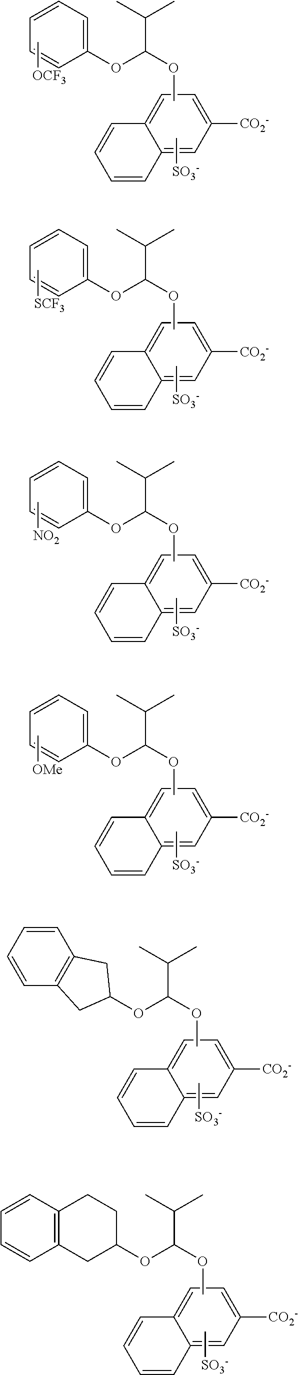

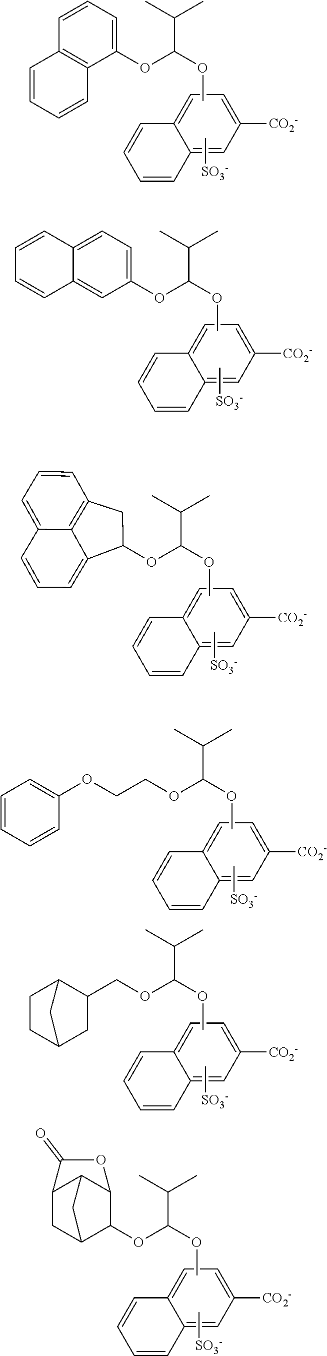

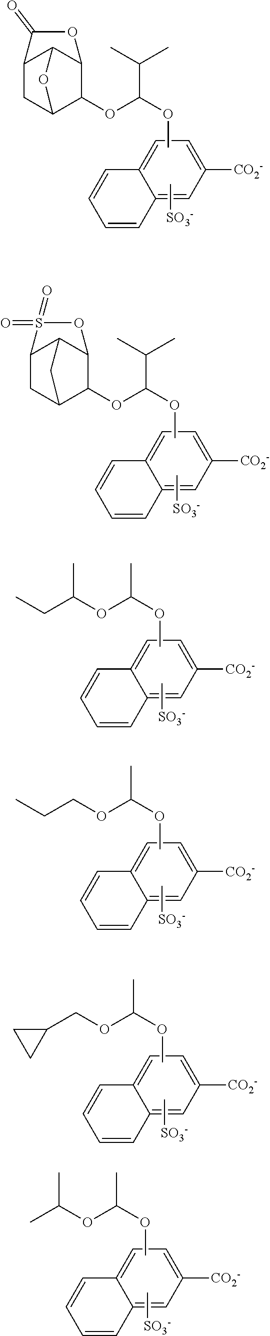

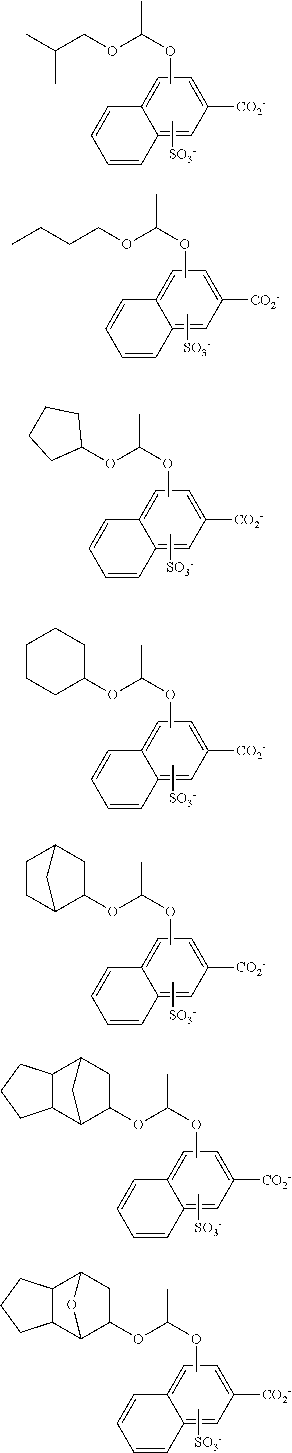

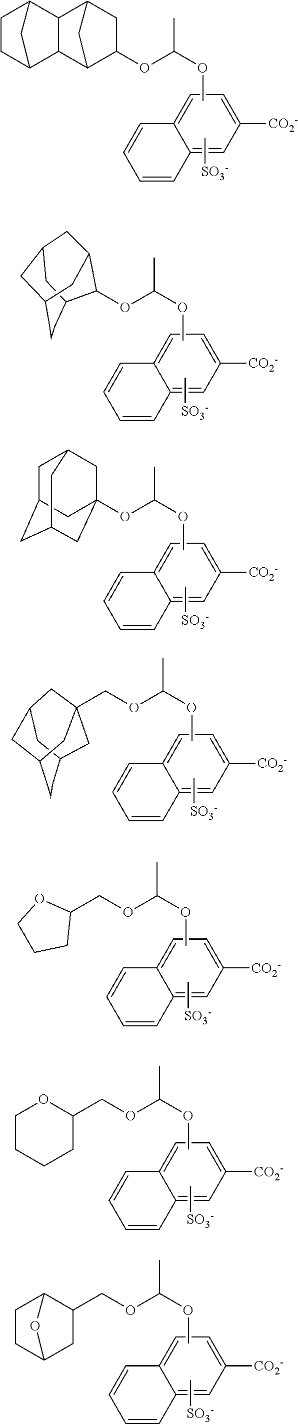

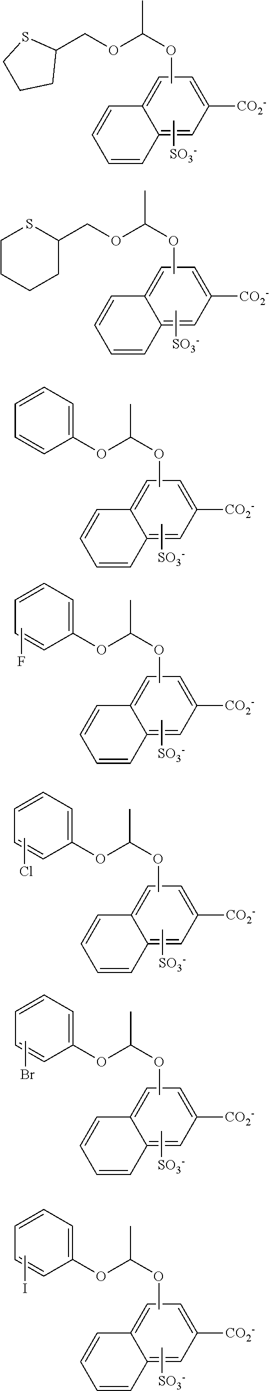

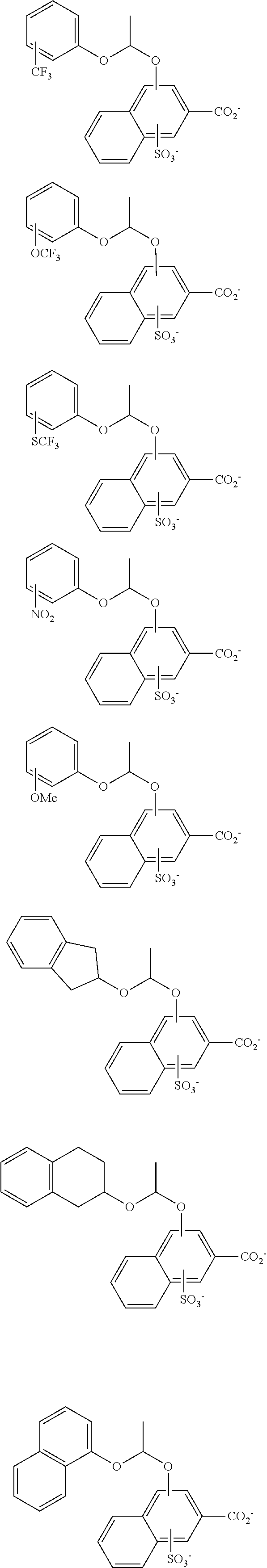

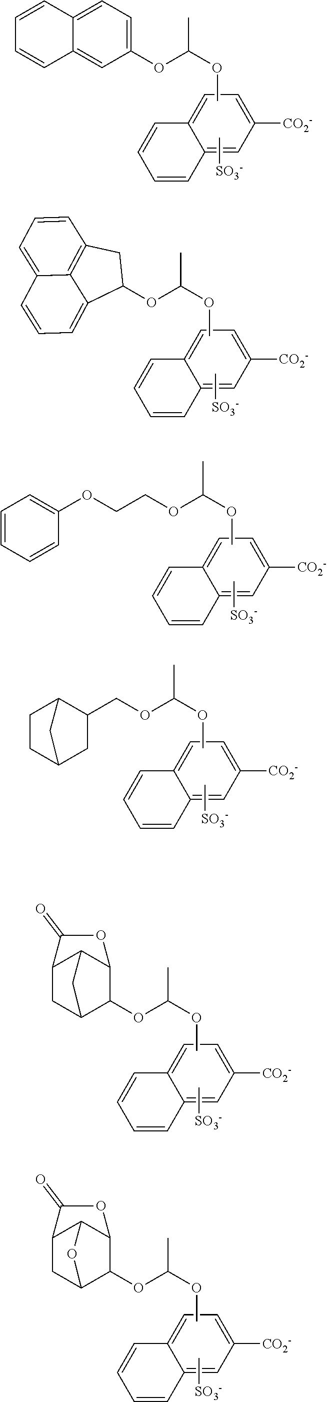

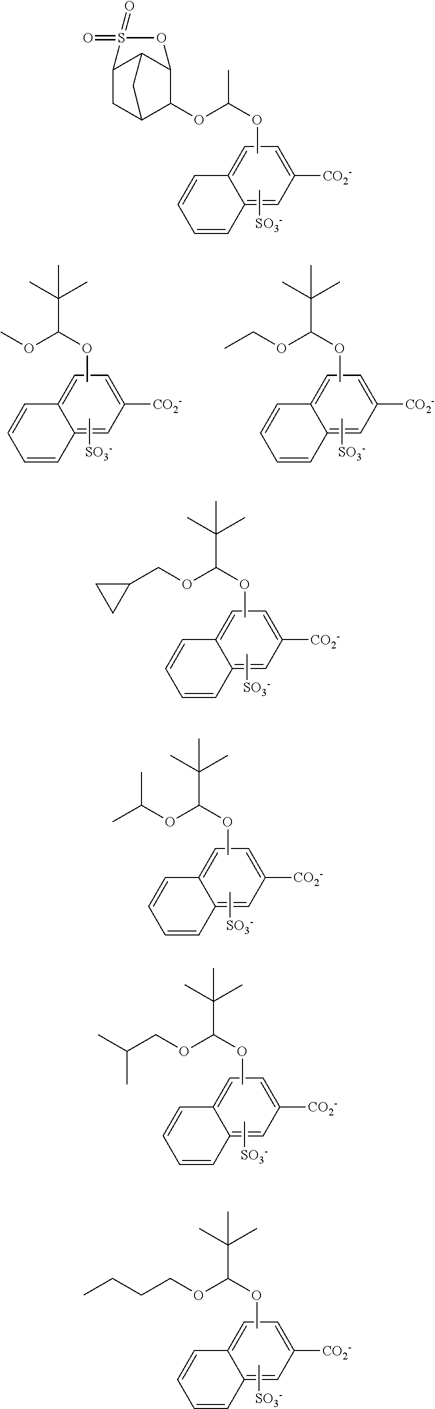

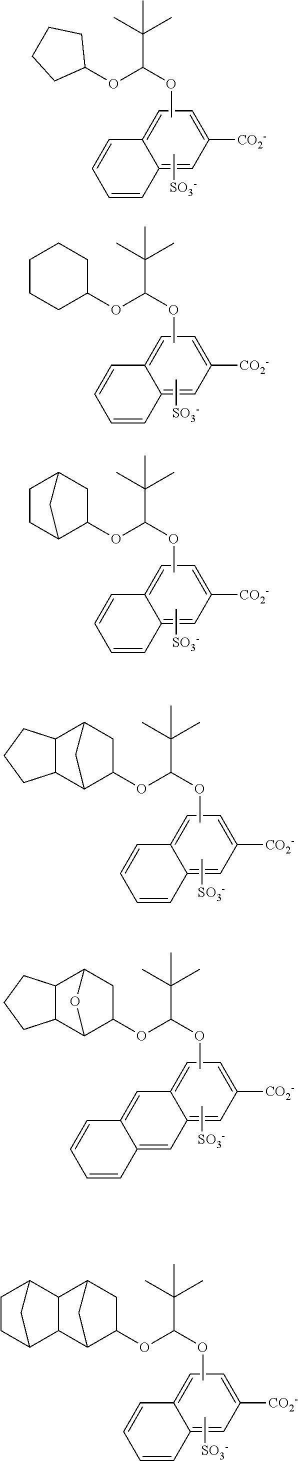

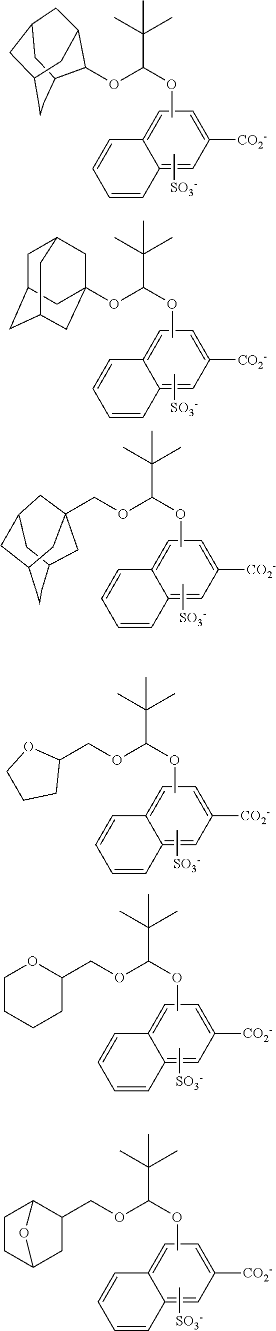

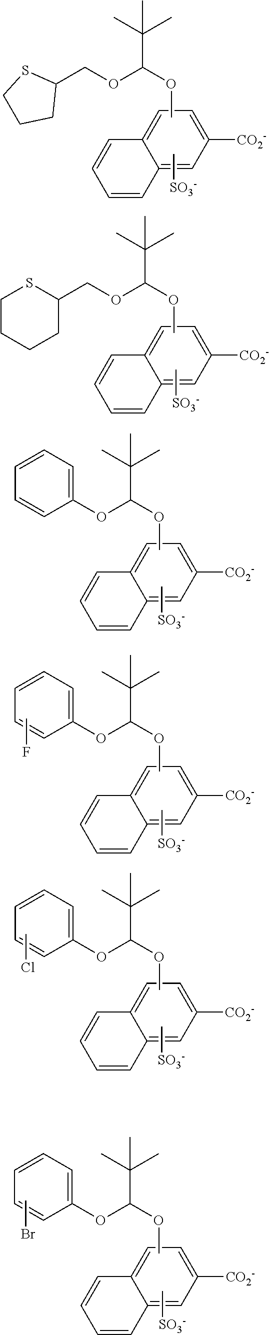

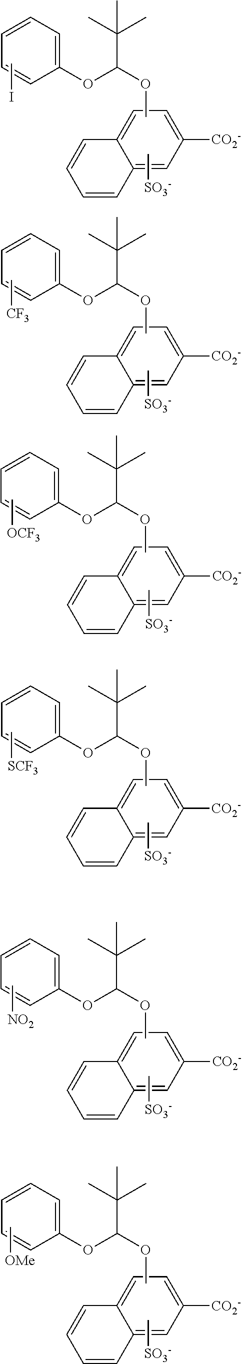

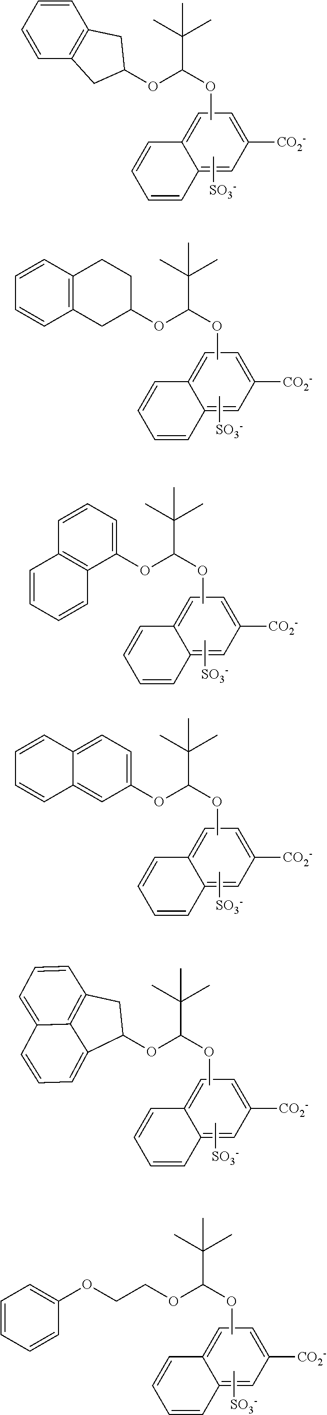

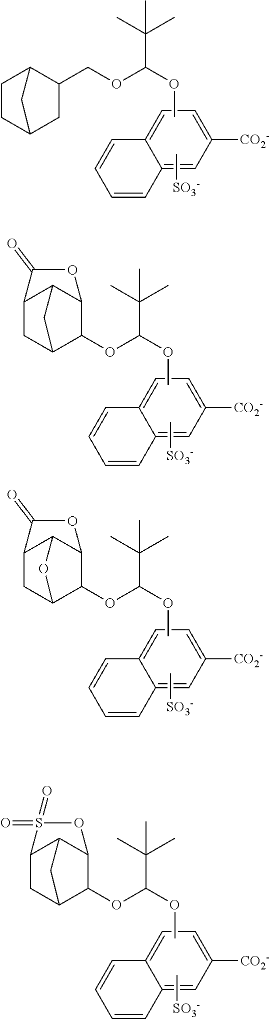

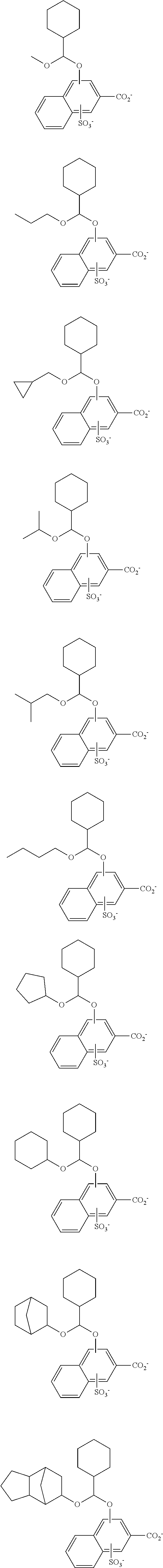

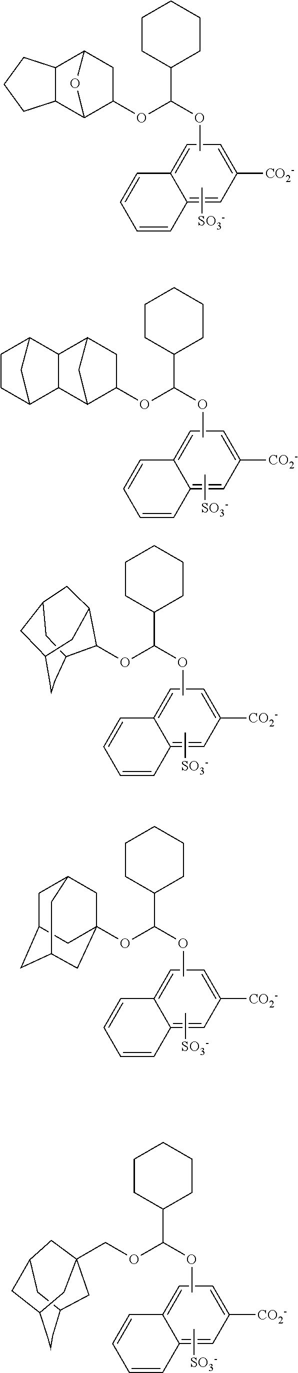

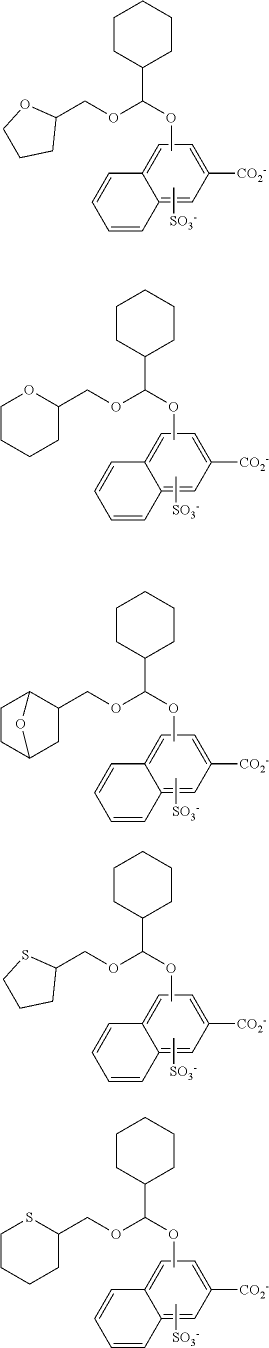

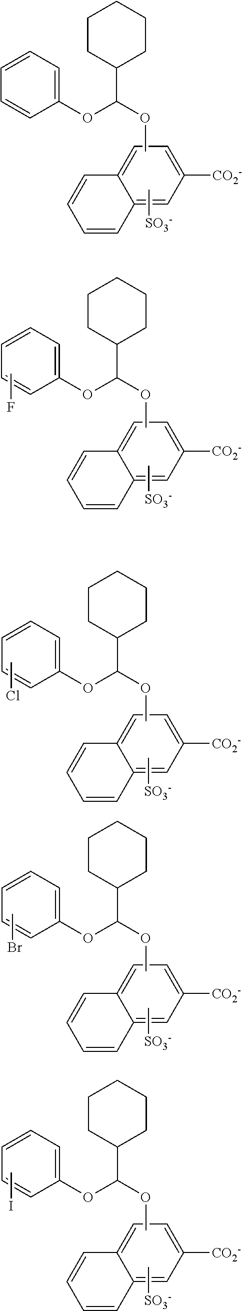

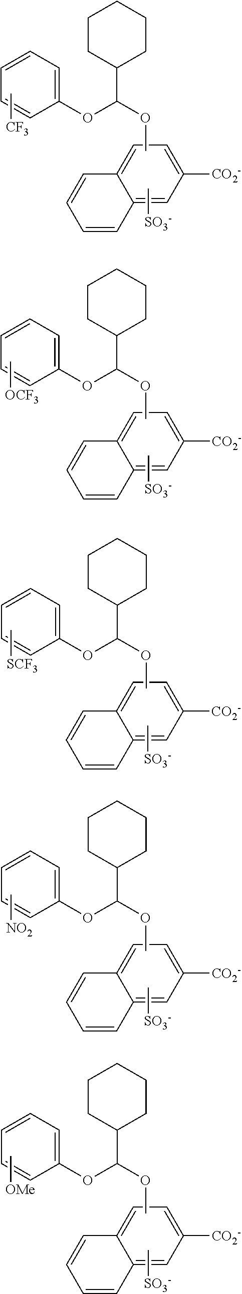

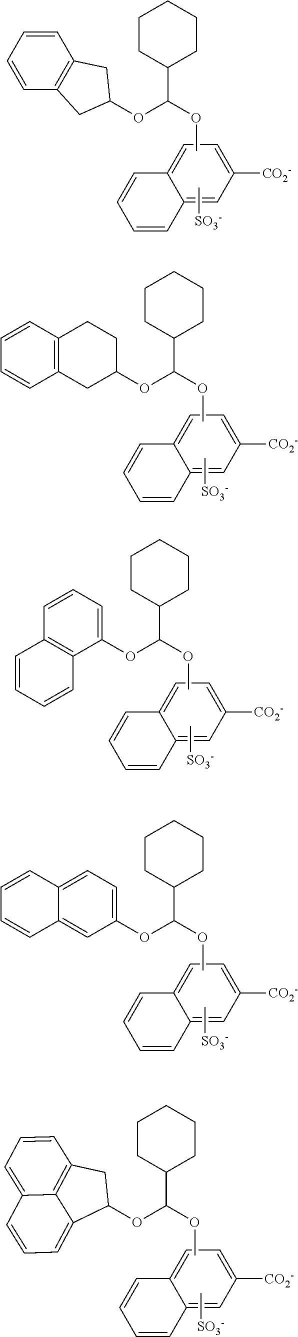

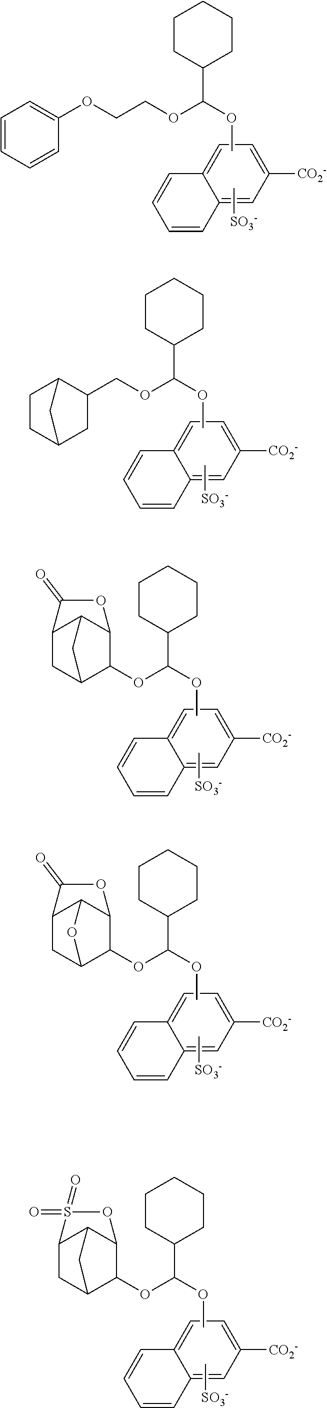

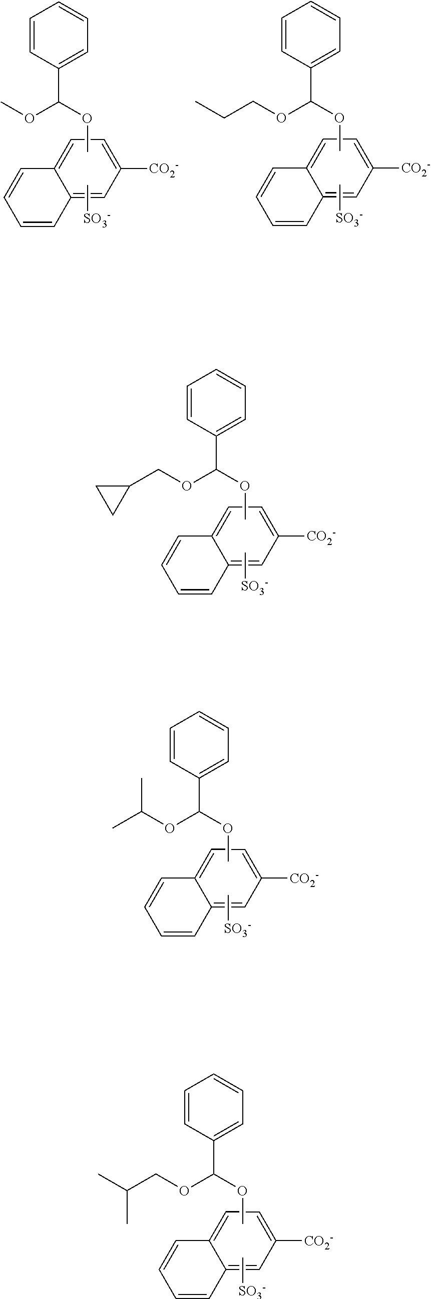

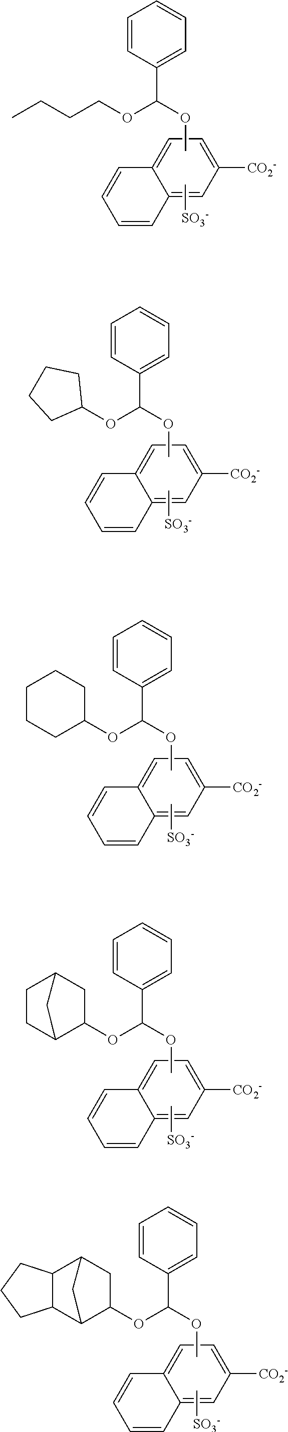

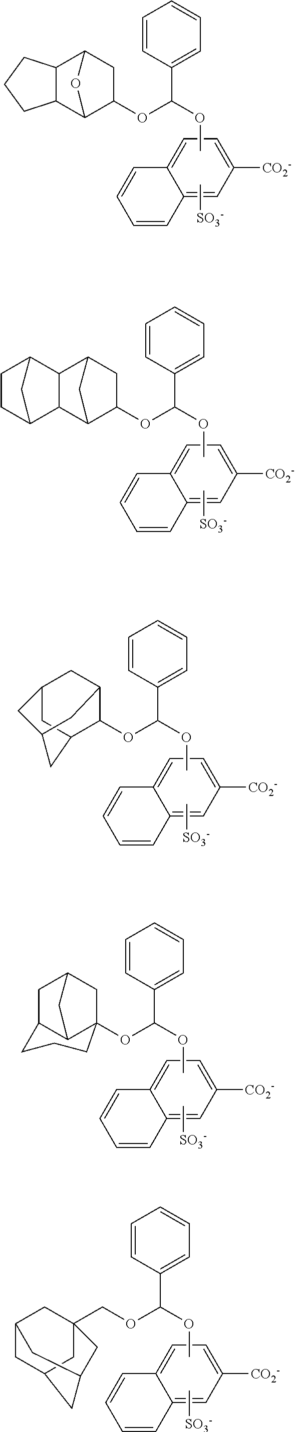

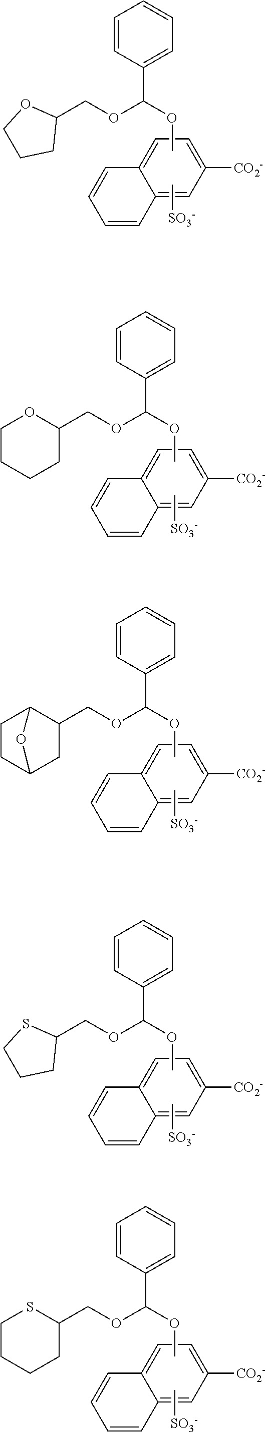

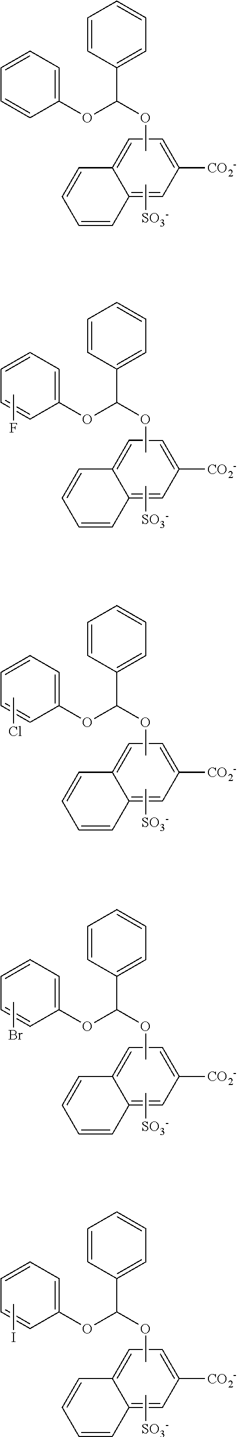

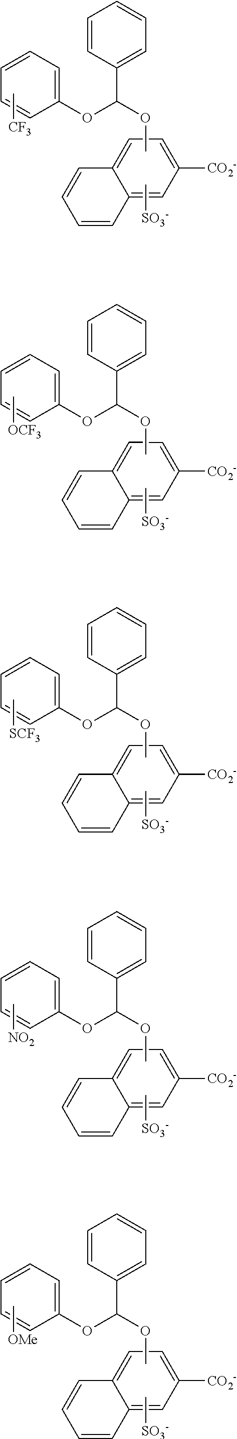

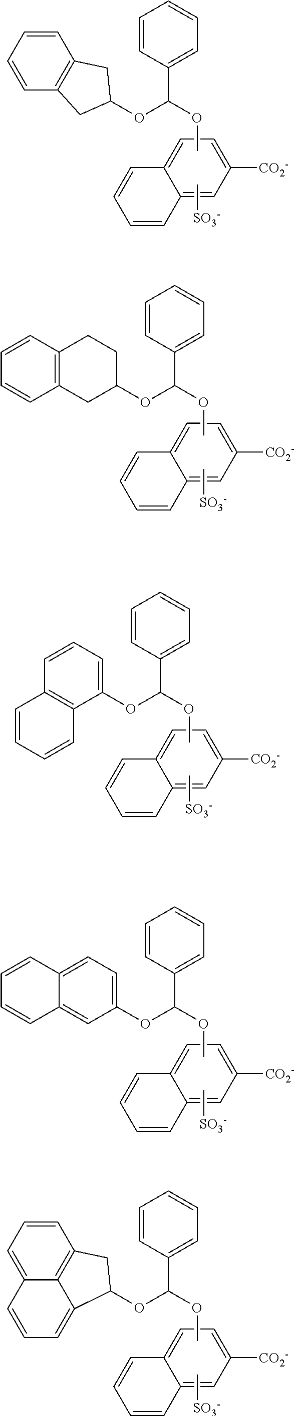

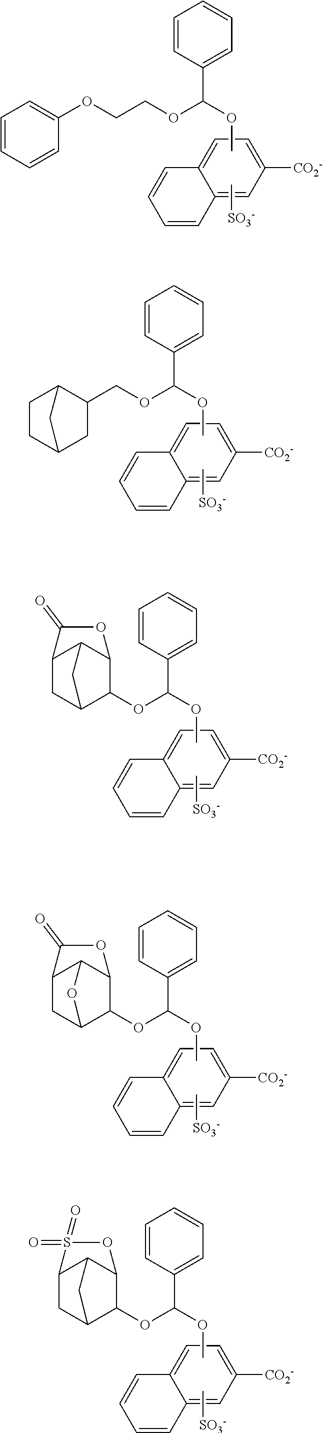

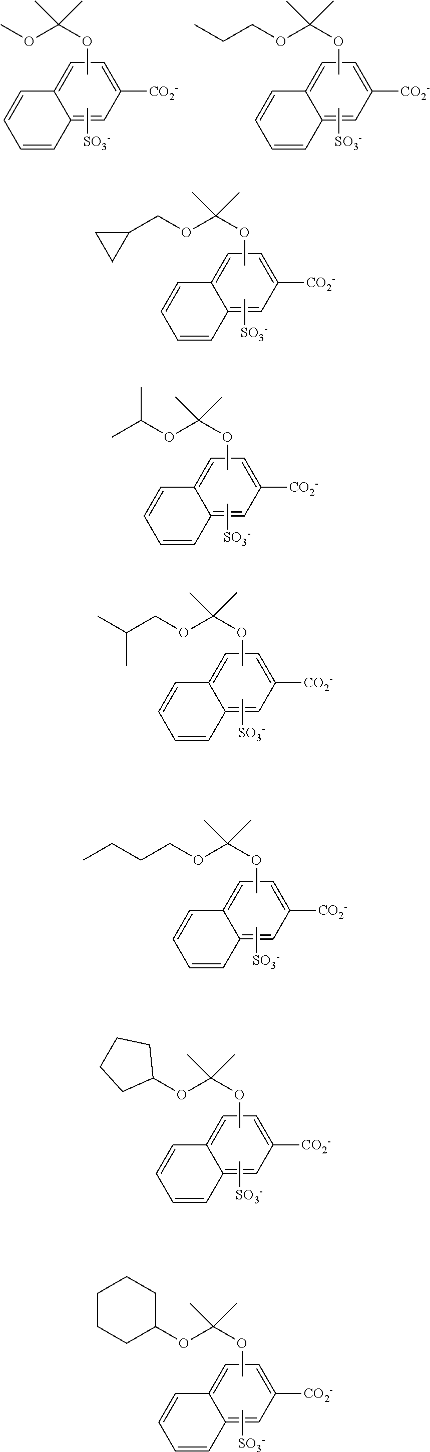

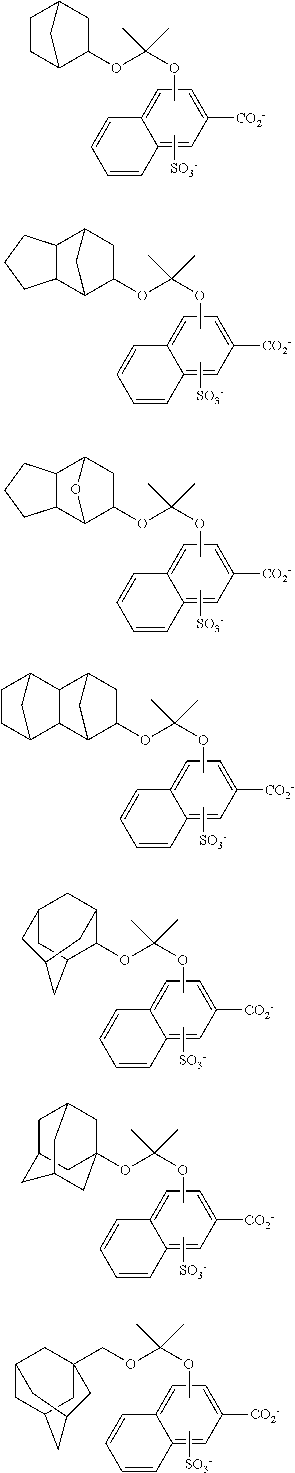

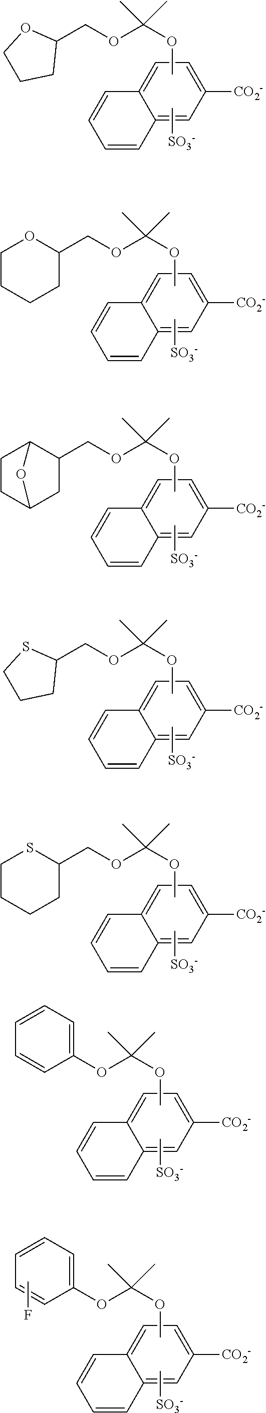

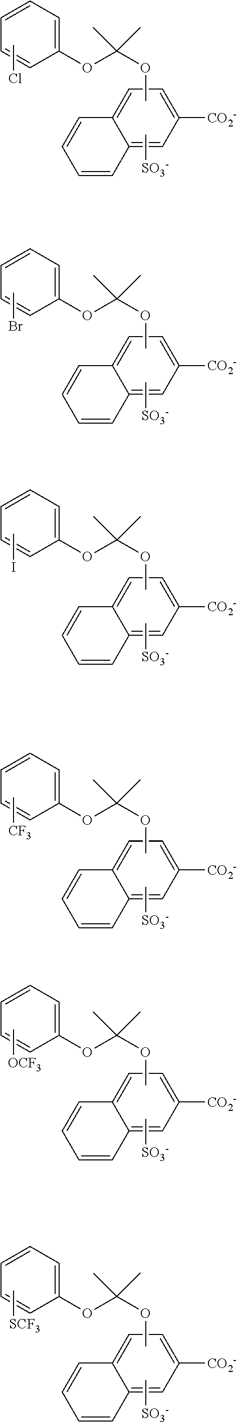

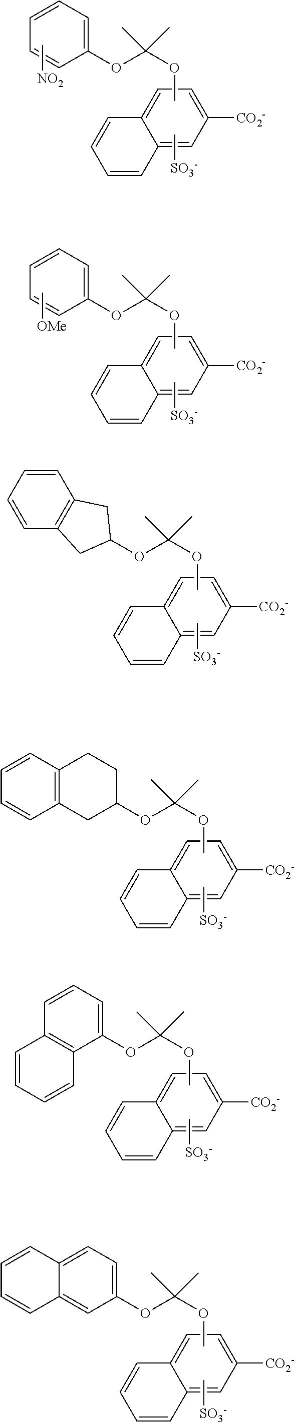

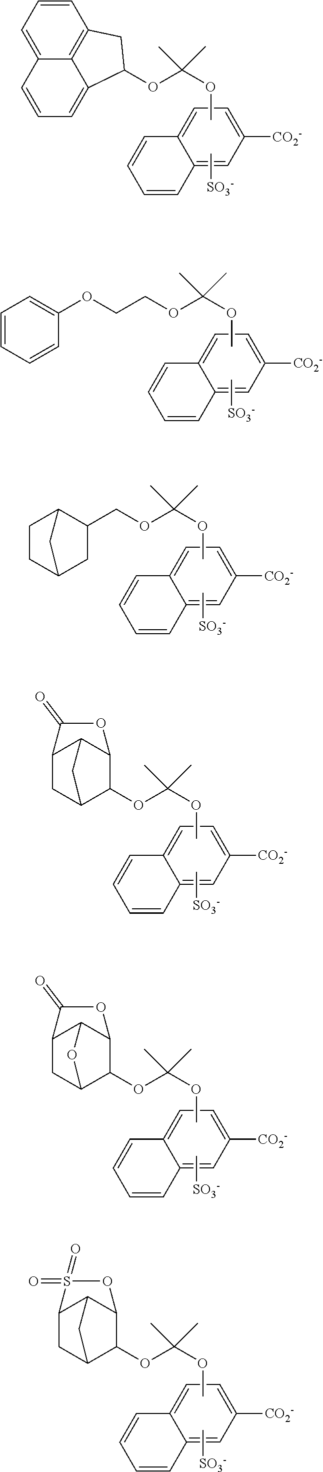

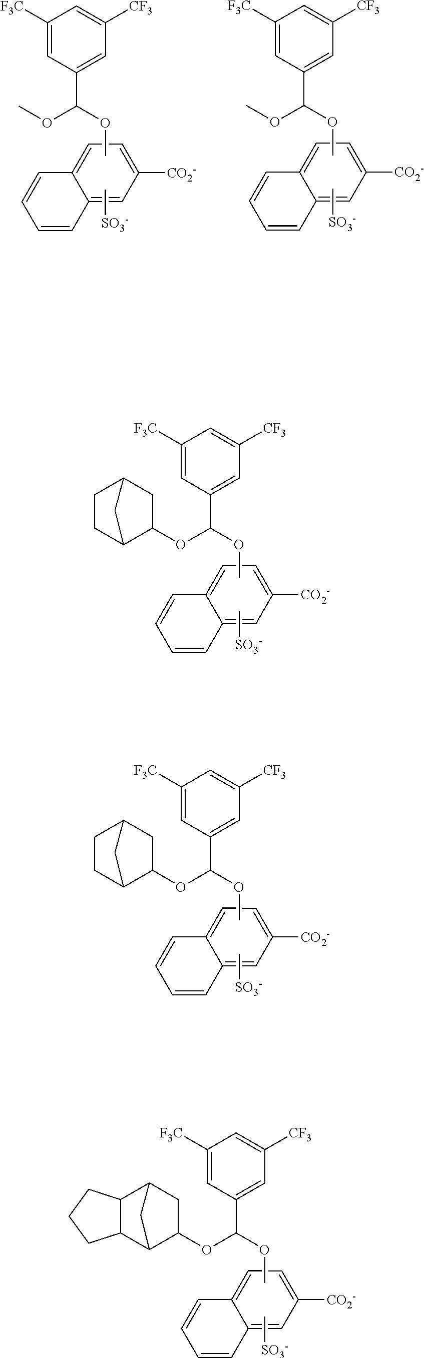

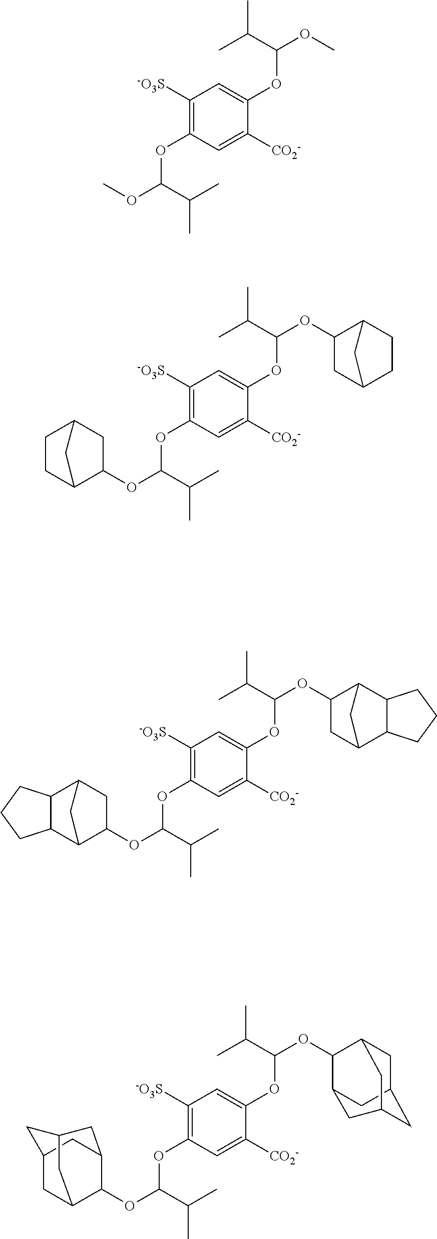

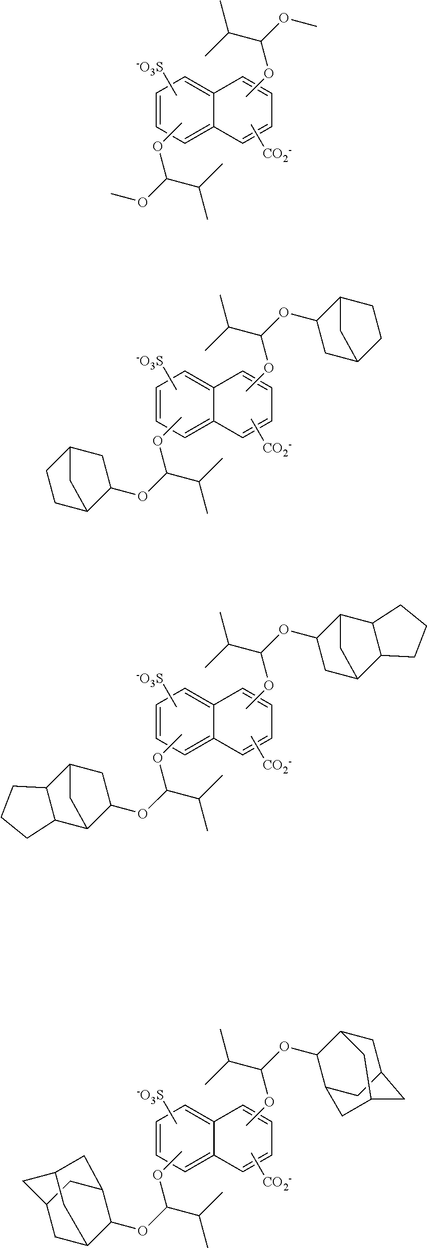

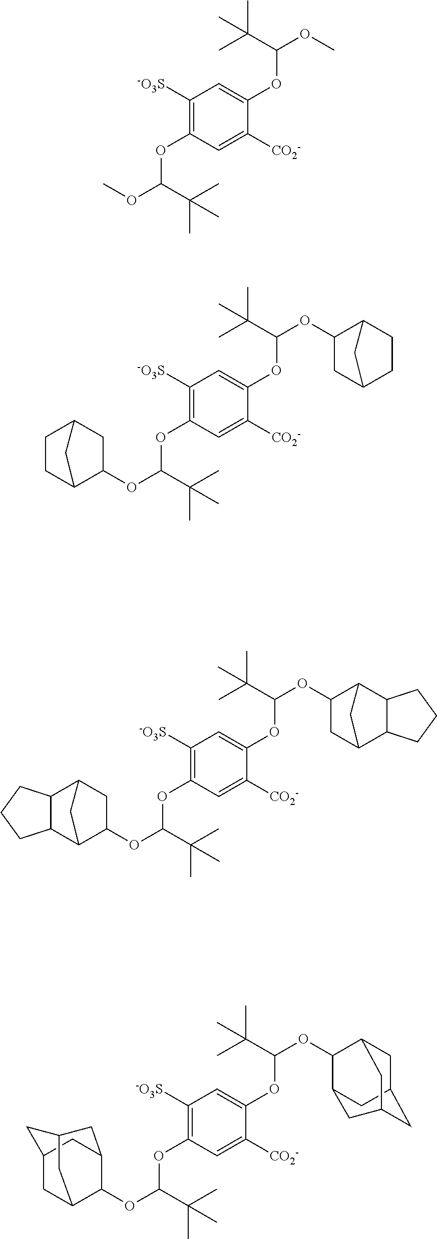

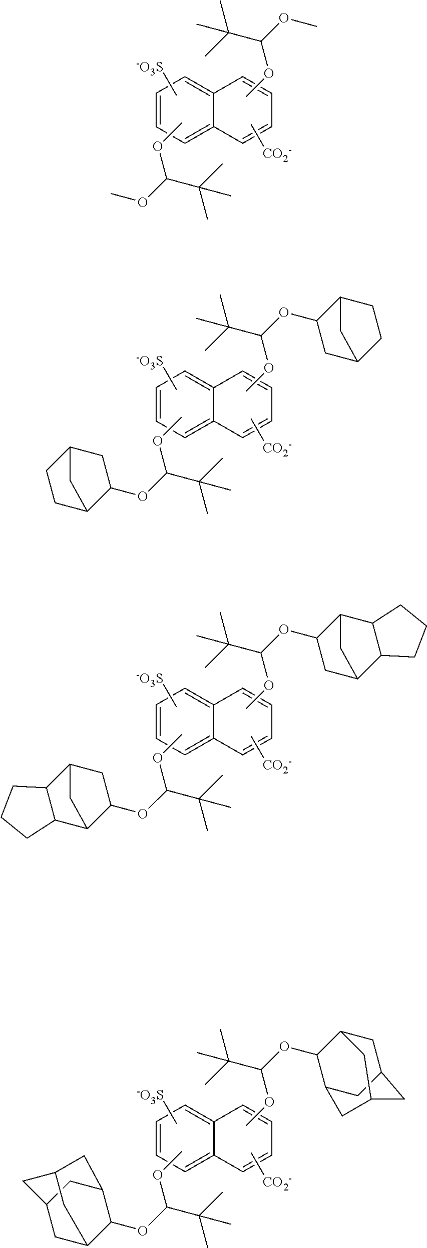

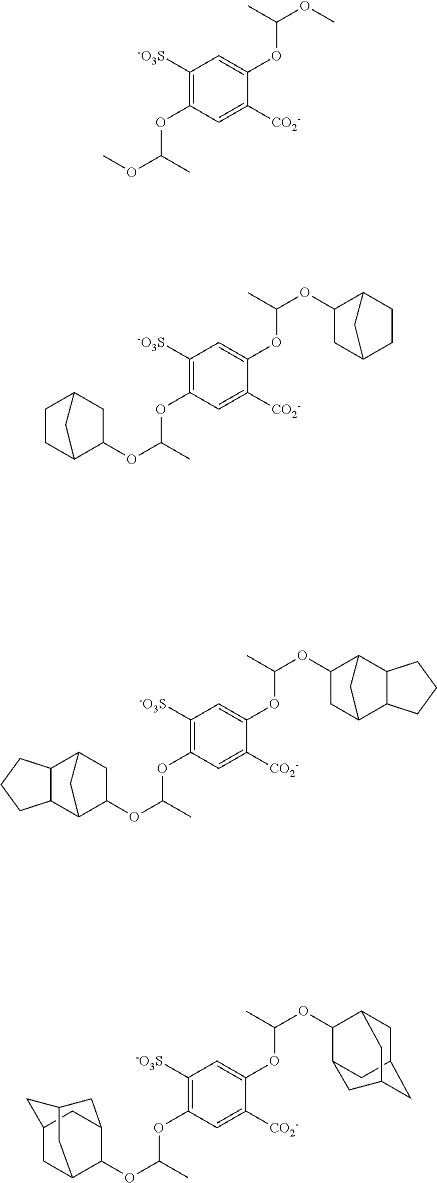

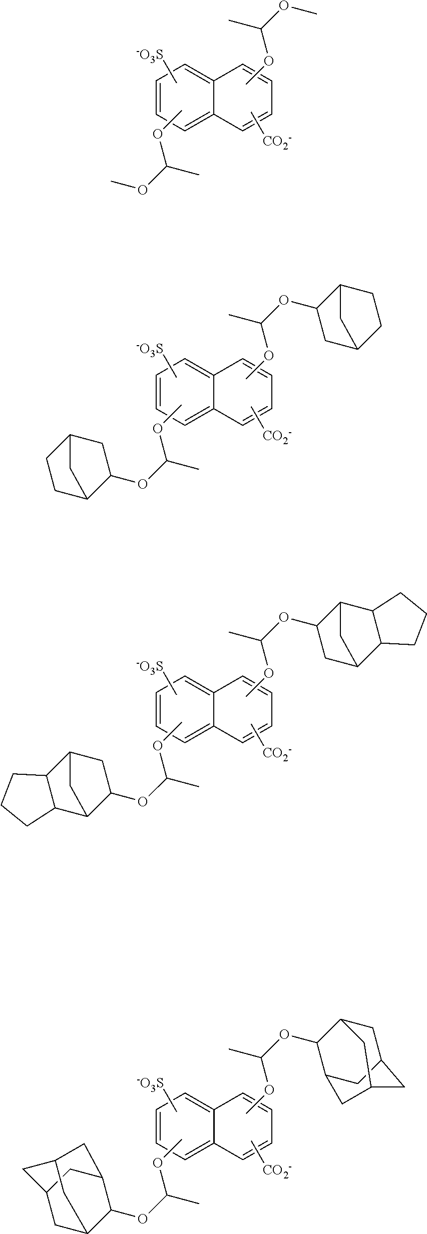

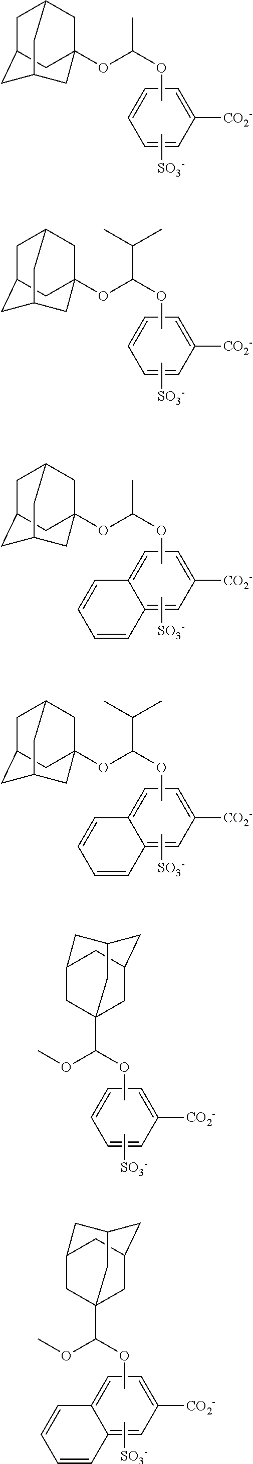

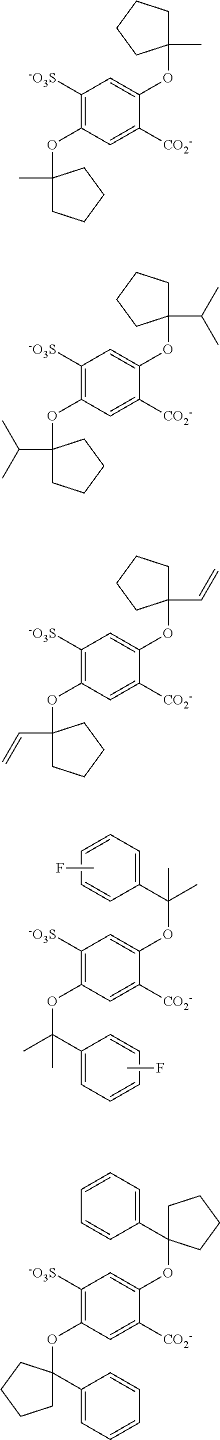

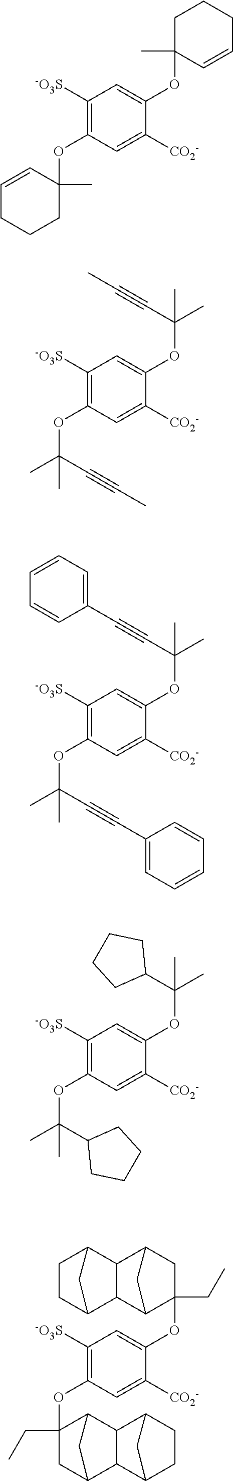

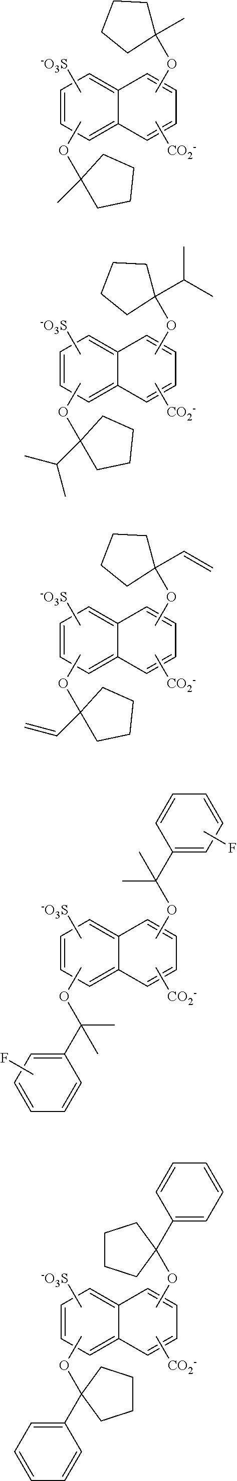

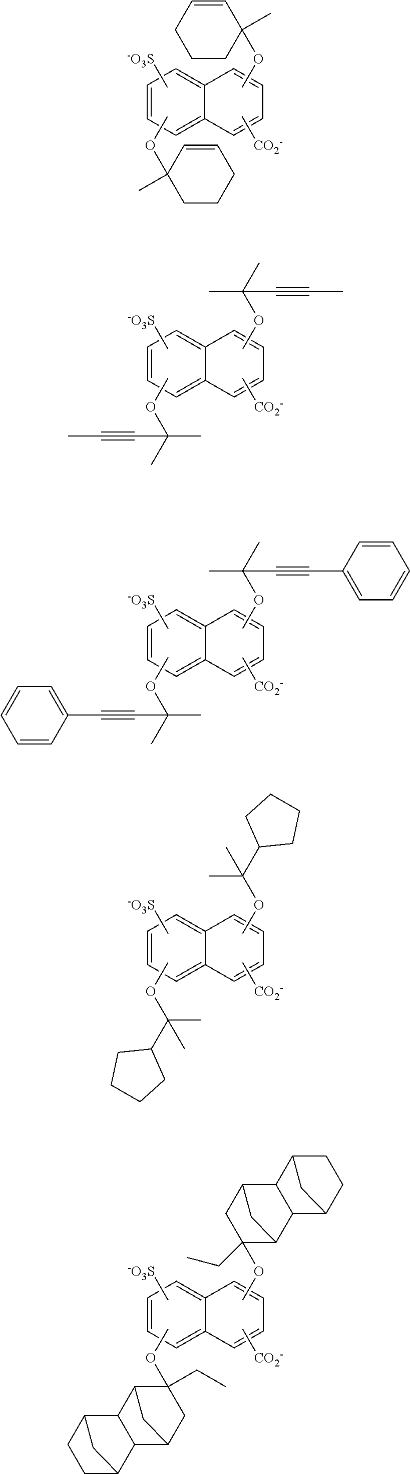

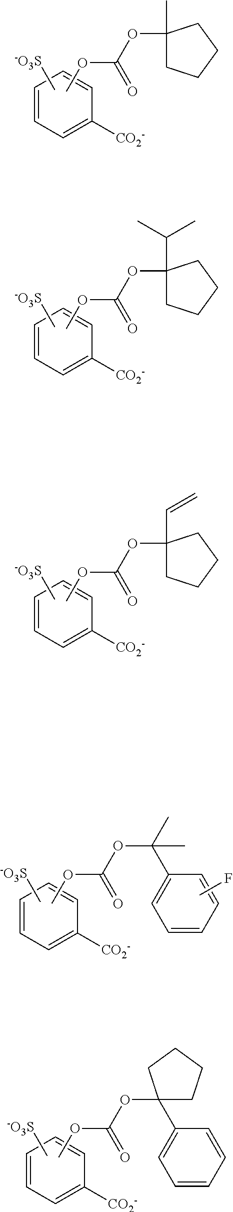

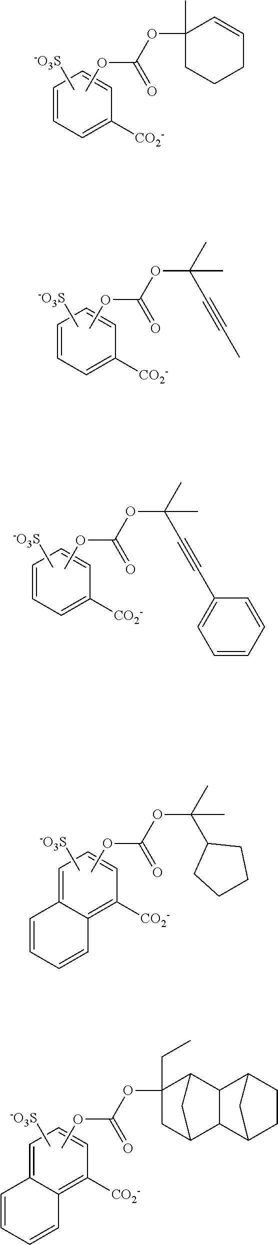

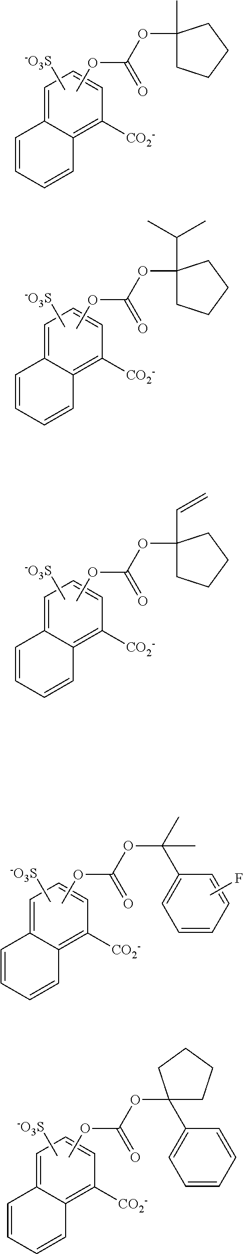

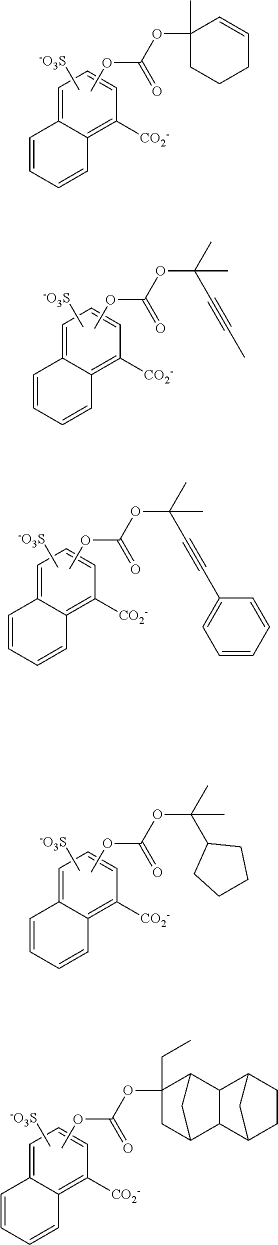

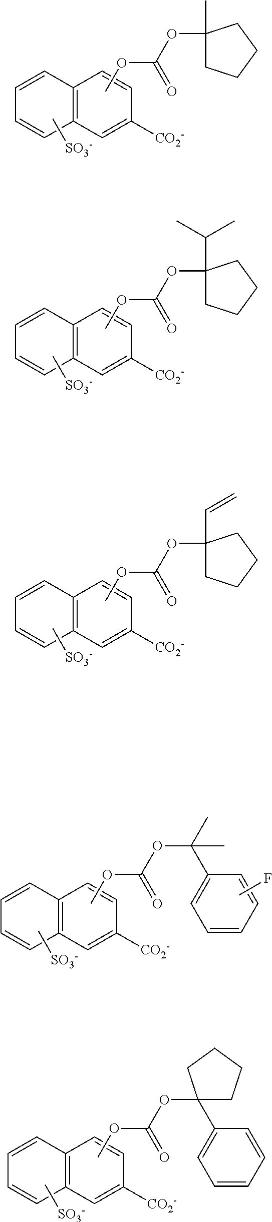

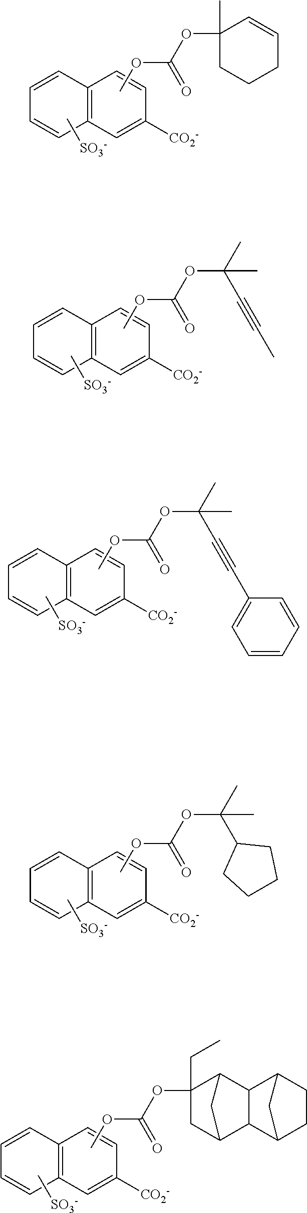

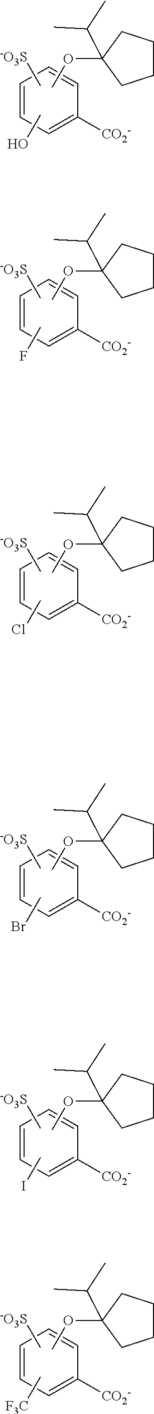

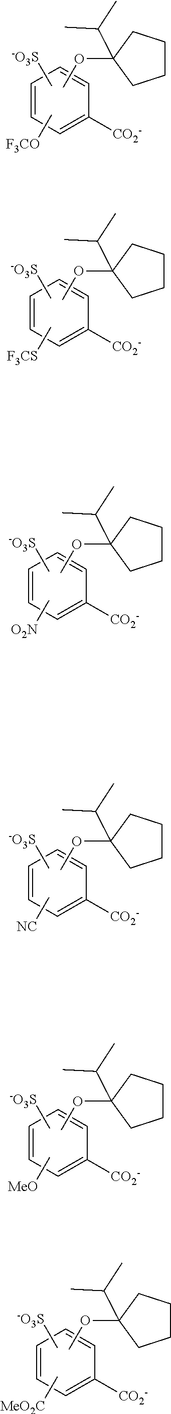

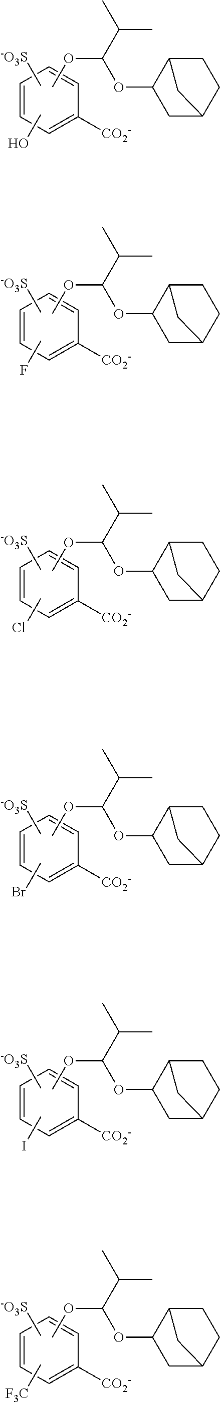

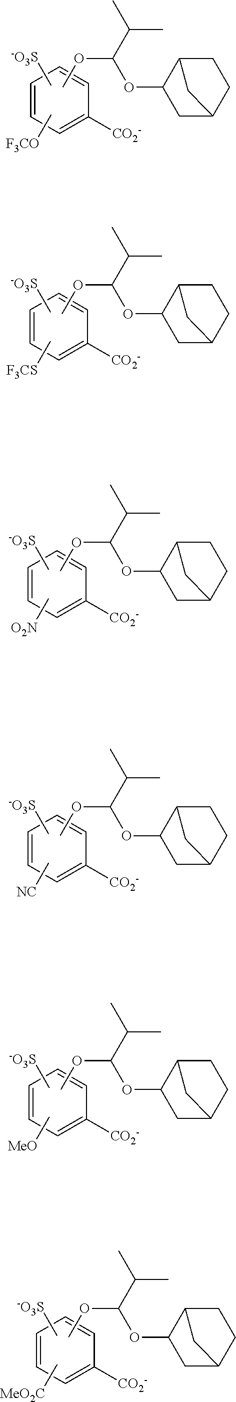

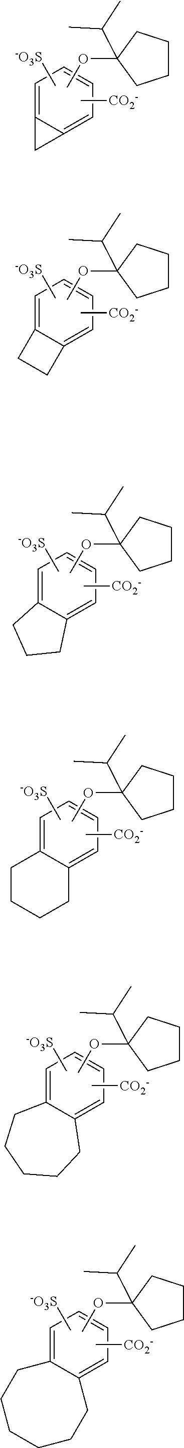

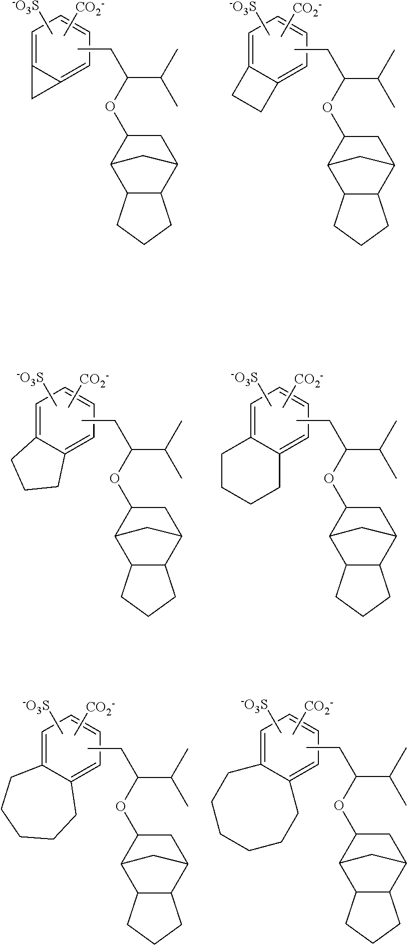

In formula (1), Z⁺ is each independently an onium cation. Examples of the onium cation include a sulfonium cation, an iodonium cation, an ammonium cation, and a phosphonium cation, and in particular, a sulfonium cation having the formula (Z-1), an iodonium cation having the following formula (Z-2), or an ammonium cation having the formula (Z-3) is preferred.

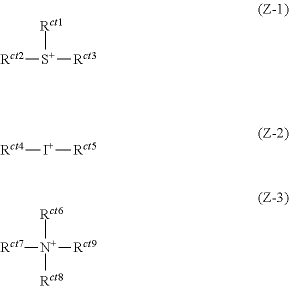

In formulae (Z-1) to (Z-3), R^{ct1} to R^{ct9} are each independently a halogen atom or a C₁-C₃₀ hydrocarbyl group which may contain a heteroatom.

Specific examples of the halogen atoms represented by R^{ct1} to R^{ct9} include a fluorine atom, a chlorine atom, a bromine atom, and an iodine atom.

The hydrocarbyl groups R^{ct1} to R^{ct9} may be saturated or unsaturated and straight, branched, or cyclic. Specific examples thereof include C₁-C₃₀ alkyl groups such as a methyl group, an ethyl group, an n-propyl group, an isopropyl group, an n-butyl group, an isobutyl group, a sec-butyl group, and a tert-butyl group; C₃-C₃₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₃₀ alkenyl groups such as a vinyl group, a 1-propenyl group, a 2-propenyl group, a butenyl group, and a hexenyl group; C₃-C₃₀ cyclic unsaturated hydrocarbyl groups such as a cyclohexenyl group; C₆-C₃₀ aryl groups such as a phenyl group, a naphthyl group, and a thienyl group; C₇-C₃₀ aralkyl groups such as a benzyl group, a 1-phenylethyl group, and a 2-phenylethyl group; and combinations thereof, and aryl groups are preferred. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

R^{ct1} and R^{ct2} may bond together to form a ring with the sulfur atom to which they are attached. Specific examples of the structure of the ring are shown below.

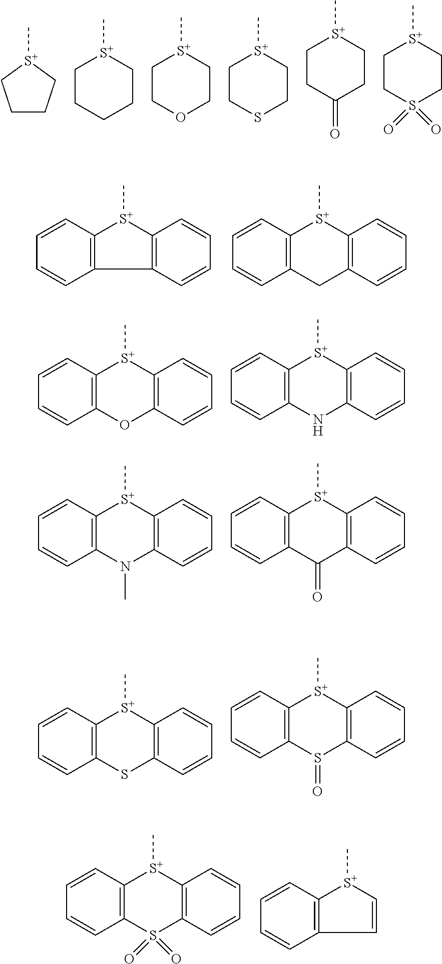

Herein the broken line designates a point of attachment to R^{ct3}.

Any two of R^{ct6} to R^{ct9} may bond together to form a ring with the nitrogen atoms to which they are attached.

Specific examples of the sulfonium cation having formula (Z-1) include those described in paragraphs [0102] to [0125] of JP-A 2024-003744 and those described in paragraphs [0070] to [0085] of JP-A 2023-169812, but are not limited thereto.

Specific examples of the iodonium cation having formula (Z-2) include those described in paragraph [0181] of JP-A 2024-000259, but are not limited thereto.

Specific examples of the ammonium cation having formula (Z-3) include are shown below, but not limited thereto.

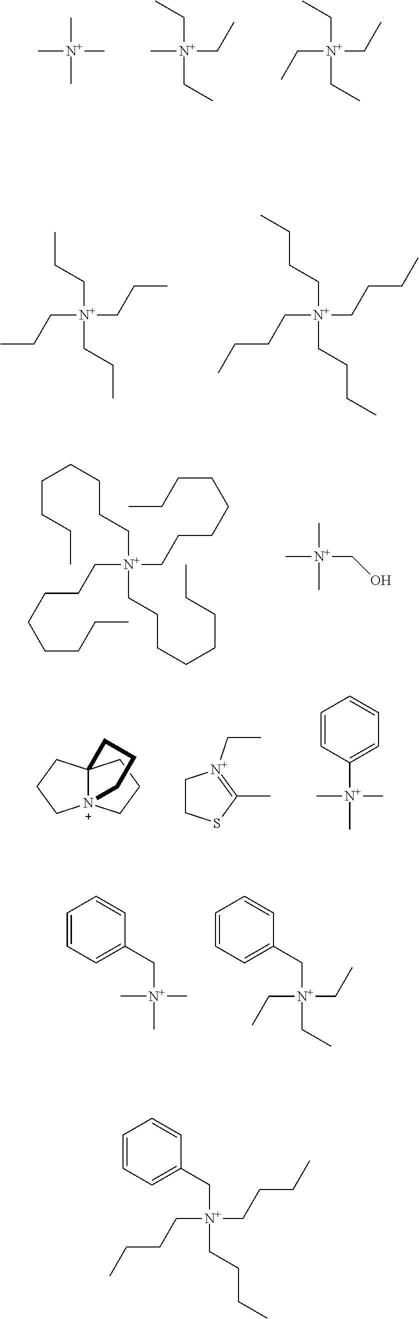

As the onium cation represented by Z⁺, a sulfonium cation having the formula (Z-4) is also preferred.

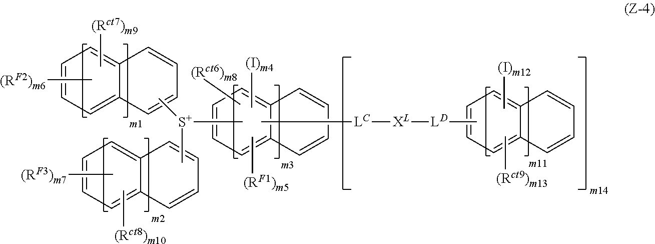

In formula (Z-4), m1 is 0 or 1. The relevant structure is a benzene ring when m1=0, and a naphthalene ring when m1=1. From the viewpoint of solvent solubility, the benzene ring corresponding to m1-0 is preferred. m2 is 0 or 1. The relevant structure is a benzene ring when m2=0, and a naphthalene ring when m2=1. From the viewpoint of solvent solubility, the benzene ring corresponding to m2=0 is preferred. m3 is 0 or 1. The relevant structure is a benzene ring when m3-0, and a naphthalene ring when m3=1. From the viewpoint of solvent solubility, the benzene ring corresponding to m3=0 is preferred.

In formula (Z-4), m4 is 0, 1, 2, 3, or 4. Since a cation structure containing more iodine atoms is more absorptive to EUV, but loses solvent solubility so that it may precipitate in a resist composition, m4 is preferably 0, 1, 2 or 3, and more preferably 0, 1 or 2.

In formula (Z-4), m5 is 0, 1, 2, 3, or 4. From the viewpoint of reactant availability, m5 is preferably 0, 1, 2, or 3, and more preferably 0, 1, or 2. m6 is 0, 1, 2, 3, 4, 5, or 6. From the viewpoint of reactant availability, m6 is preferably 0, 1, 2, or 3, and more preferably 0, 1, or 2. m7 is 0, 1, 2, 3, 4, 5, or 6. From the viewpoint of reactant availability, m7 is preferably 0, 1, 2, or 3, and more preferably 0, 1, or 2.

In formula (Z-4), m8 is 0, 1, or 2. From the viewpoint of reactant availability, m8 is preferably 0 or 1. m9 is 0, 1, or 2. From the viewpoint of reactant availability, m9 is preferably 0 or 1. m10 is 0, 1, or 2. From the viewpoint of reactant availability, m10 is preferably 0 or 1.

In formula (Z-4), m11 is 0 or 1. The relevant structure is a benzene ring when m11=0, and a naphthalene ring when m11=1. From the viewpoint of solvent solubility, the benzene ring corresponding to m11=0 is preferred.

In formula (Z-3), m12 is 0, 1, 2, 3, or 4. Since a cation structure containing more iodine atoms is more absorptive to EUV, but loses solvent solubility so that it may precipitate in a resist composition, m12 is preferably 0, 1, 2 or 3, and more preferably 0, 1 or 2.

In formula (Z-4), m13 is 0, 1, or 2. From the viewpoint of reactant availability, m13 is preferably 0 or 1. m14 is 0, 1, or 2. From the viewpoint of synthesis, m14 is preferably 0 or 1.

Provided that 0≤m6+m9≤4 when m1 is 0, and 0≤m6+m9≤6 when m1 is 1, 0≤m7+m10≤4 when m2 is 0 and 0≤m7+m10≤6 when m2 is 1, 1≤m4+m5+m8+m14≤4 when m3 is 0 and 1≤m4+m5+m8+m14≤6 when m3 is 1, 0≤m12+m13≤4 when m11 is 0 and 0≤m12+m13≤6 when m11 is 1, and m4+m12≥1.

In formula (Z-4), R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group. Among them, a trifluoromethyl group, a trifluoromethoxy group, and a trifluorothiomethoxy group are preferred. A plurality of R^{F1} may be identical or different when m5 is 2 or more, a plurality of R^{F2} may be identical or different when m6 is 2 or more, a plurality of R^{F3} may be identical or different when m7 is 2 or more.

In formula (Z-4), R^{ct6} to R^{ct9} each are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom. The hydrocarbyl group and the hydrocarbyl moiety in the hydrocarbyloxy group and the hydrocarbylthio group may be saturated or unsaturated and straight, branched, or cyclic. Specific examples thereof are as exemplified above for the hydrocarbyl group R¹ in the description of formula (1). In the hydrocarbyl group and the hydrocarbyl moiety in the hydrocarbyloxy group and the hydrocarbylthio group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

When m8 is 2, two R^{ct6} may be identical or different and two R^{ct6} may bond together to form a ring with the carbon atoms to which they are attached, when m9 is 2, two R^{ct7} may be identical or different and two R^{ct7} may bond together to form a ring with the carbon atoms to which they are attached, when m10 is 2, two R^{ct8} may be identical or different and two R^{ct8} may bond together to form a ring with the carbon atoms to which they are attached, and when m13 is 2, two R^{ct9} may be identical or different and two R^{ct9} may bond together to form a ring with the carbon atoms to which they are attached. Specific examples of the ring thus formed include a cyclopropane ring, a cyclobutane ring, a cyclopentane ring, a cyclohexane ring, a norbornane ring, and an adamantane ring. In the ring, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

The aromatic rings directly bonded to S⁺ in the sulfonium cation having formula (Z-4) may bond together to form a ring with S⁺. Specific examples of the structure of the ring are shown below.

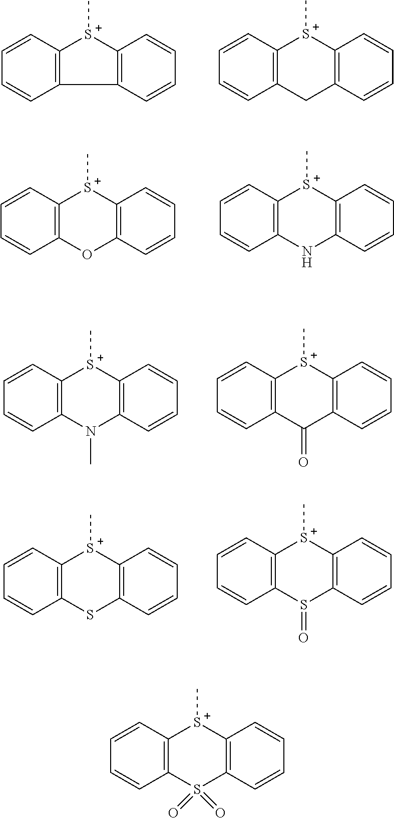

Herein the broken line designates a point of attachment.

In formula (Z-4), L^{C} and L^{D} are each independently a single bond, an ether bond, an ester bond, an amide bond, a sulfonate ester bond, a sulfonamide bond, a carbonate bond, or a carbamate bond. Among them, L^{C} is preferably a single bond, an ether bond, an ester bond, or a sulfonate ester bond, and more preferably an ester bond or a sulfonate ester bond. L^{D} is preferably a single bond, an ether bond, or an ester bond, and more preferably a single bond.

In formula (B), X^{L} is a single bond or a C₁-C₄₀ hydrocarbylene group which may contain a heteroatom. The hydrocarbylene group may be straight, branched, or cyclic, and specific examples thereof include an alkanediyl group, a cyclic saturated hydrocarbylene group, and an arylene group. Specific examples of the heteroatom include an oxygen atom, a nitrogen atom, and a sulfur atom.

Specific examples of the C₁-C₄₀ hydrocarbylene group which may contain a heteroatom represented by X^{L} are shown below, but are not limited thereto. Herein * each designates a point of attachment to L^{C} or L^{D}.

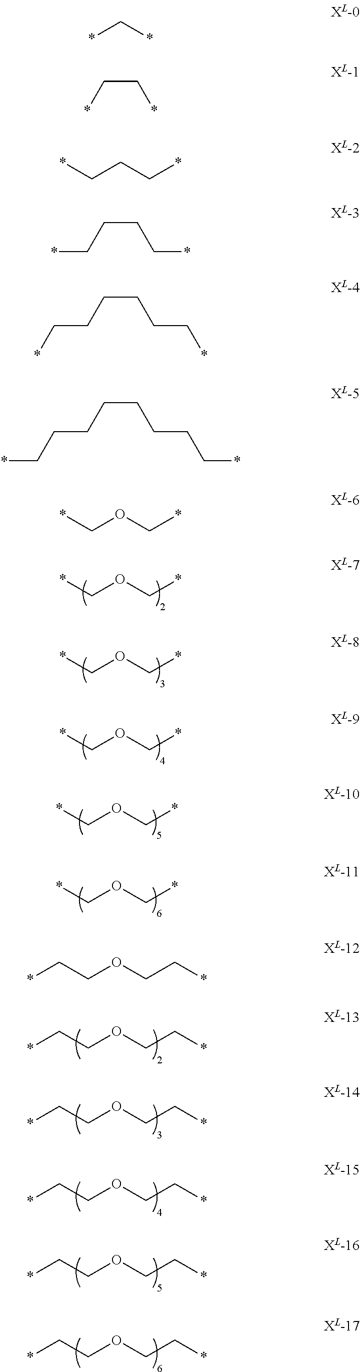

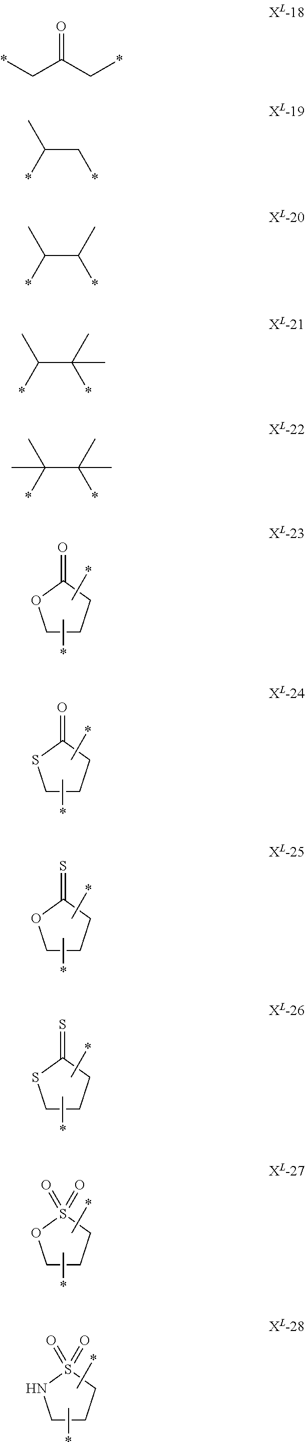

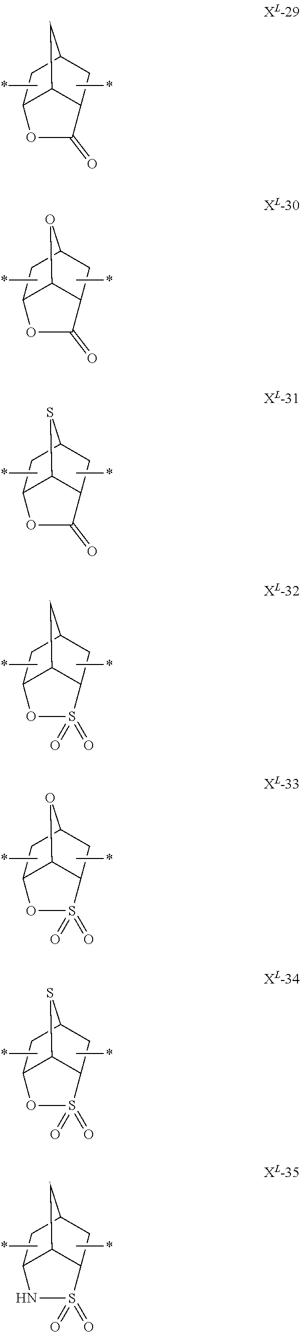

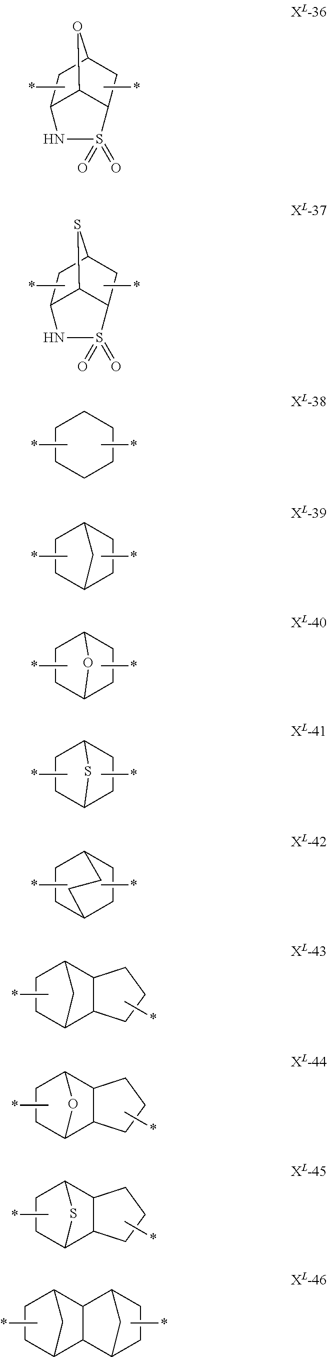

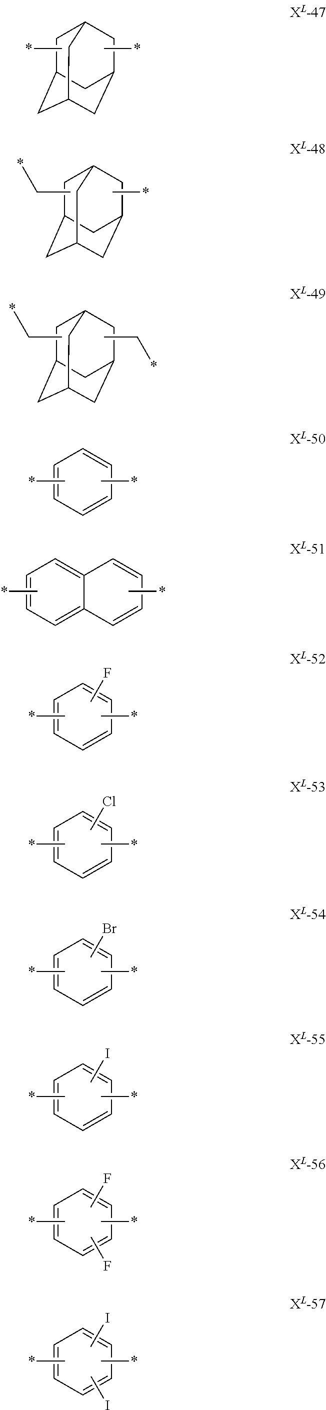

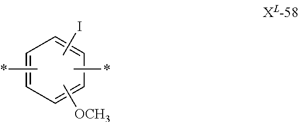

Among them, X^{L}-0 to X^{L}-22, X^{L}-29 to X^{L}-34, and X^{L}-47 to X^{L}-58 are preferred.

The sulfonium cation having formula (Z-4) is preferably a sulfonium cation having formula (Z-4-1):

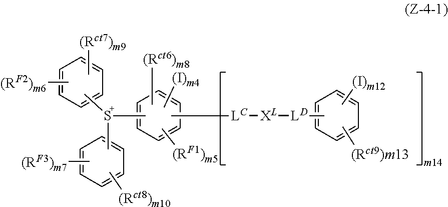

wherein m4 to m10, m12 to m14, R^{F1} to R^{F3}, R^{ct6} to R^{ct9}, L^{C}, L^{D}, and X^{L} are as defined above.

The sulfonium cation having formula (Z-4-1) is preferably a sulfonium cation having formula (Z-4-2):

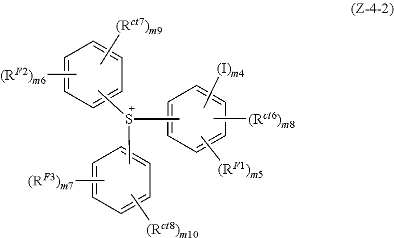

wherein m4 to m10, R^{F1} to R^{F3}, and R^{ct6} to R^{ct8} are as defined above.

Specific examples of the sulfonium cation having formula (Z-4) include are shown below, but not limited thereto. Herein Me is a methyl group.

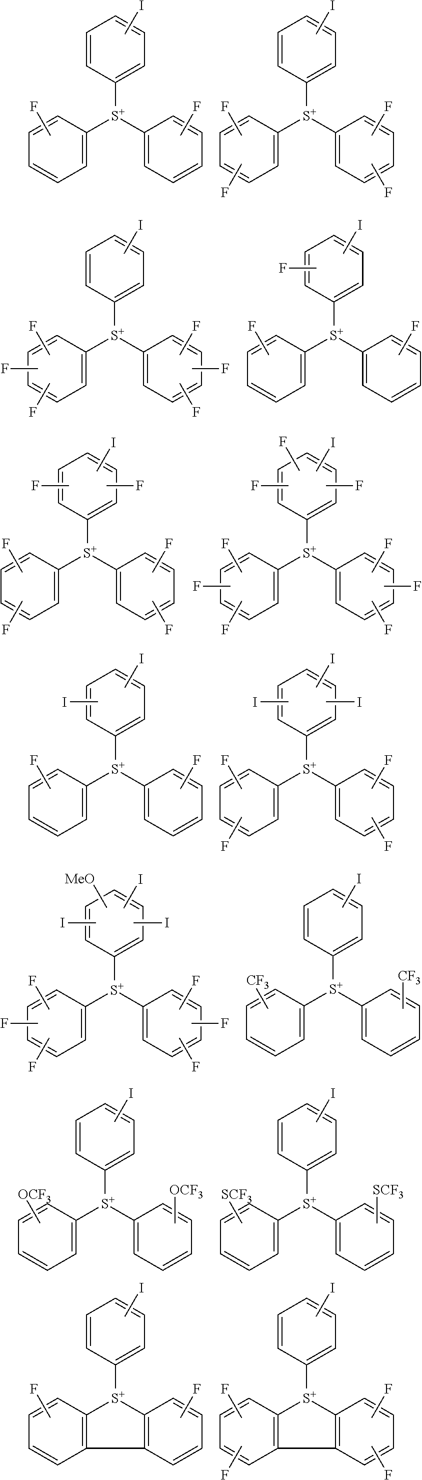

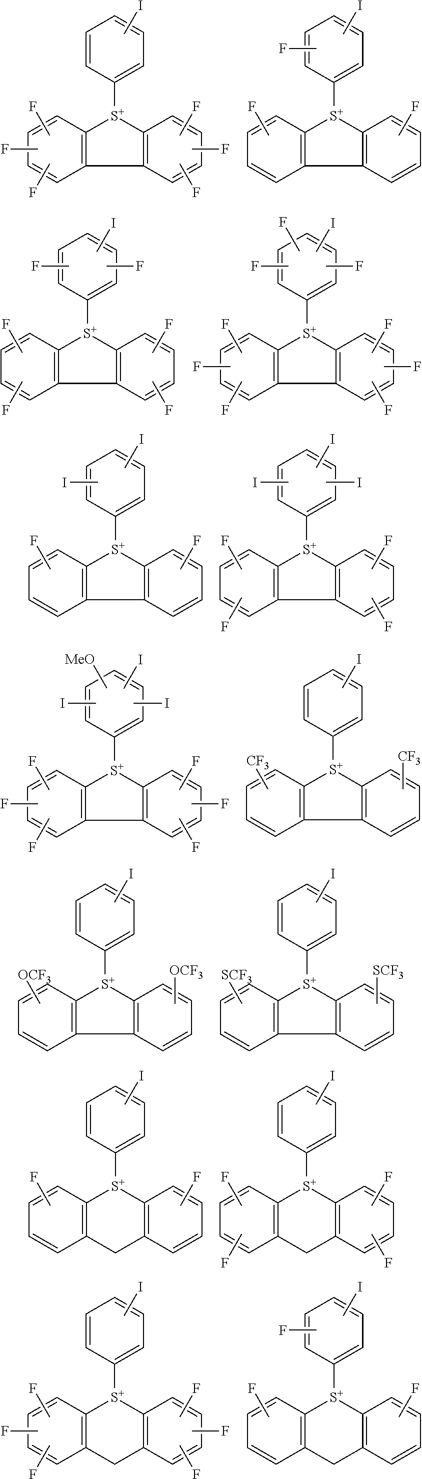

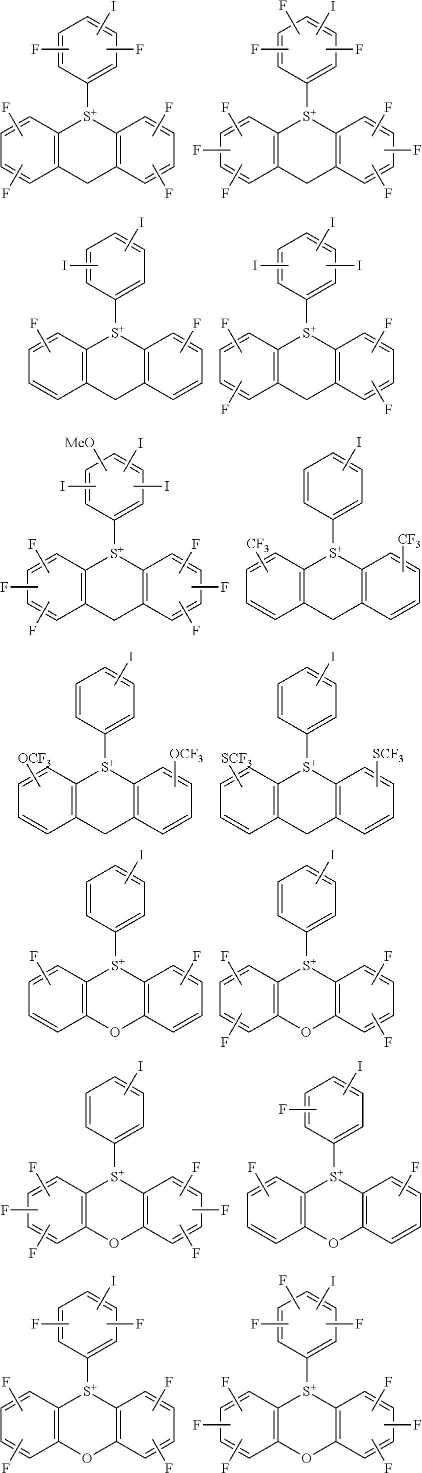

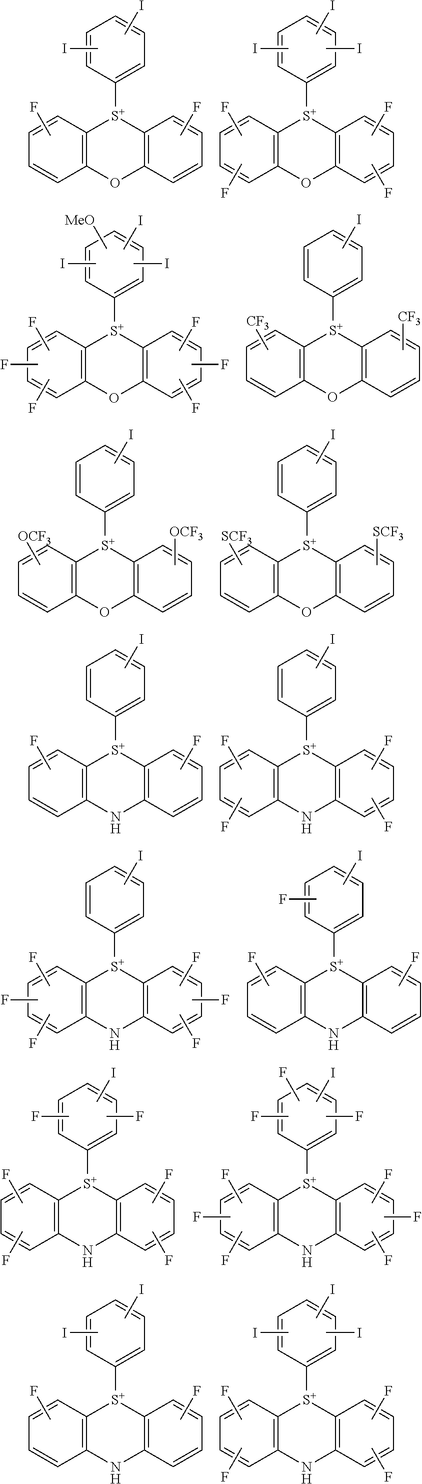

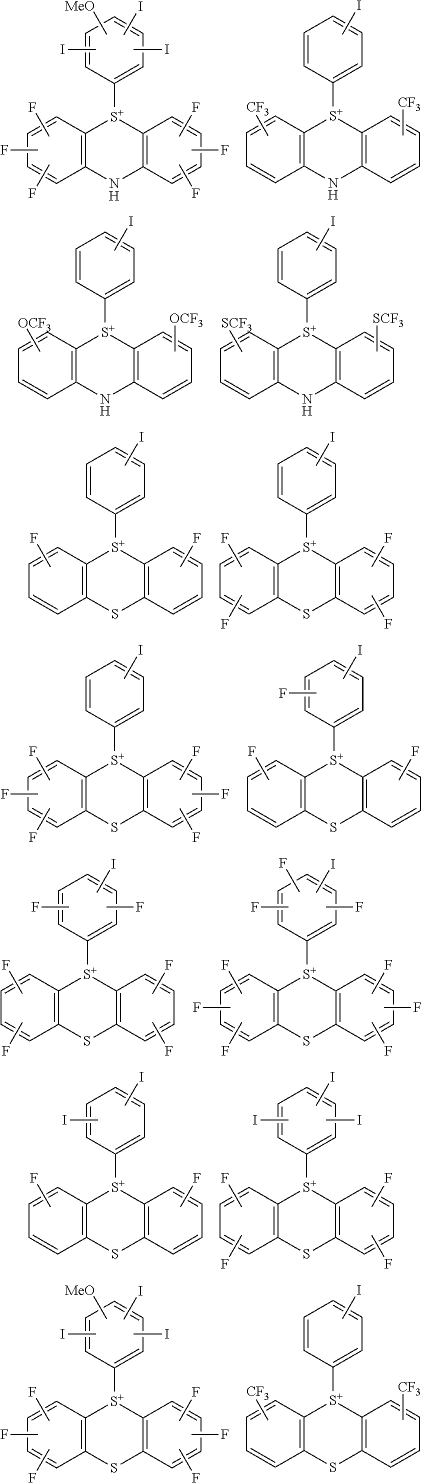

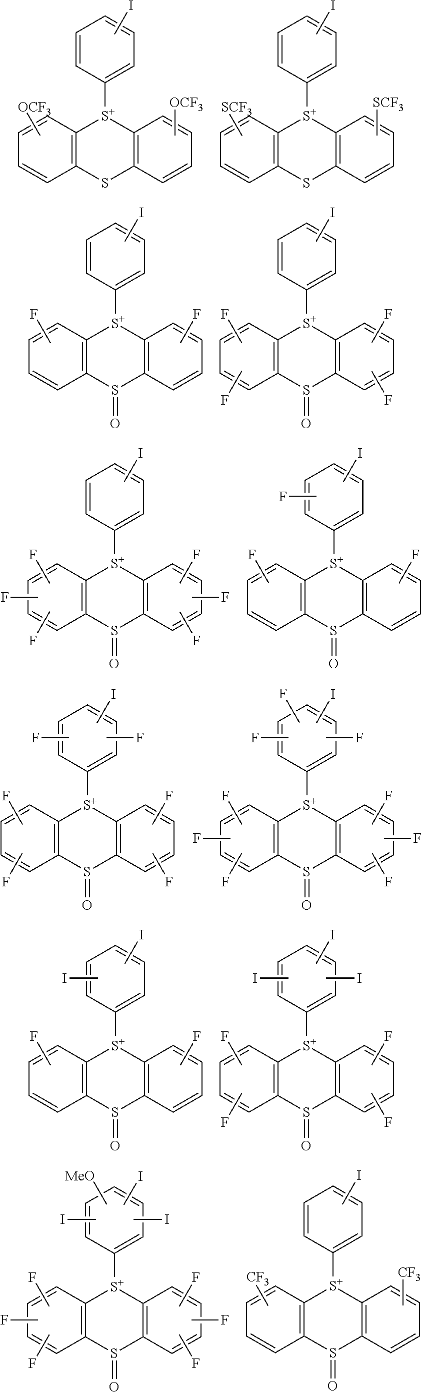

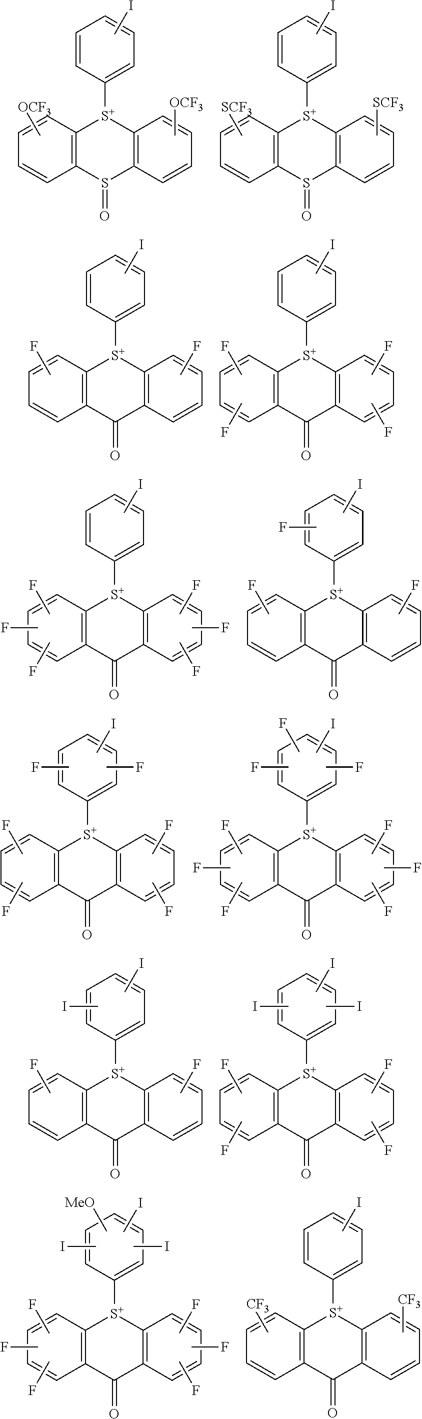

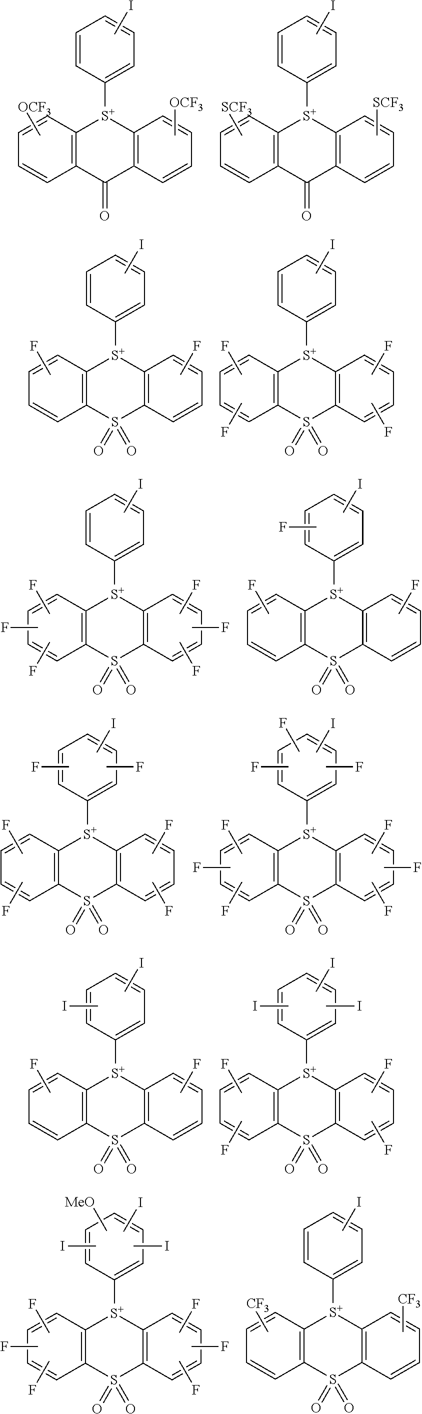

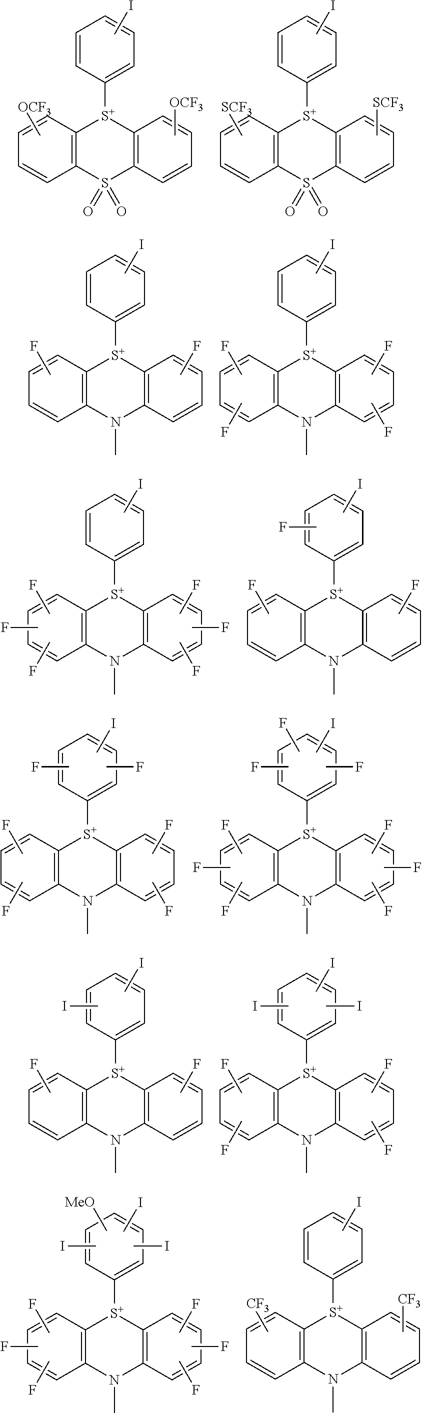

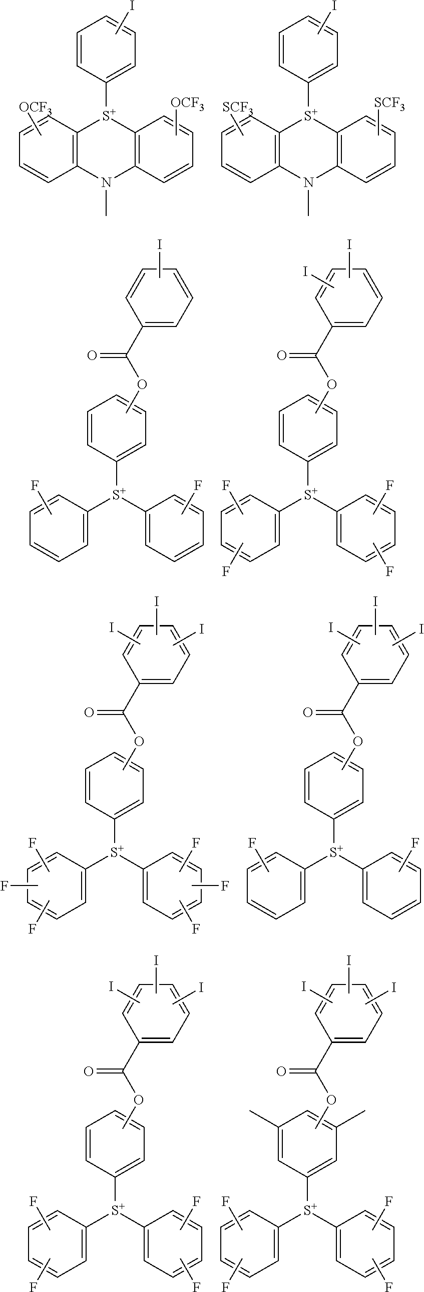

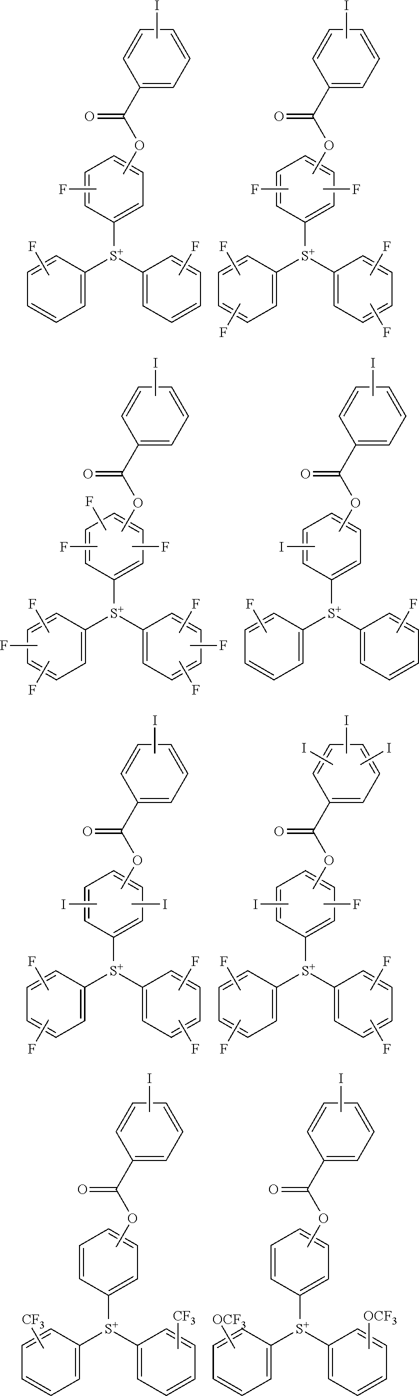

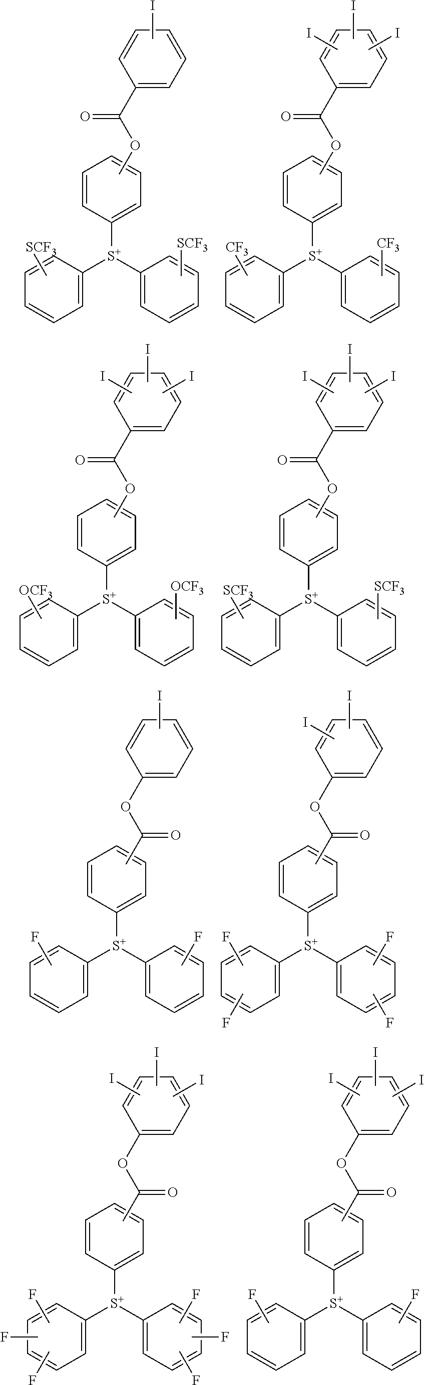

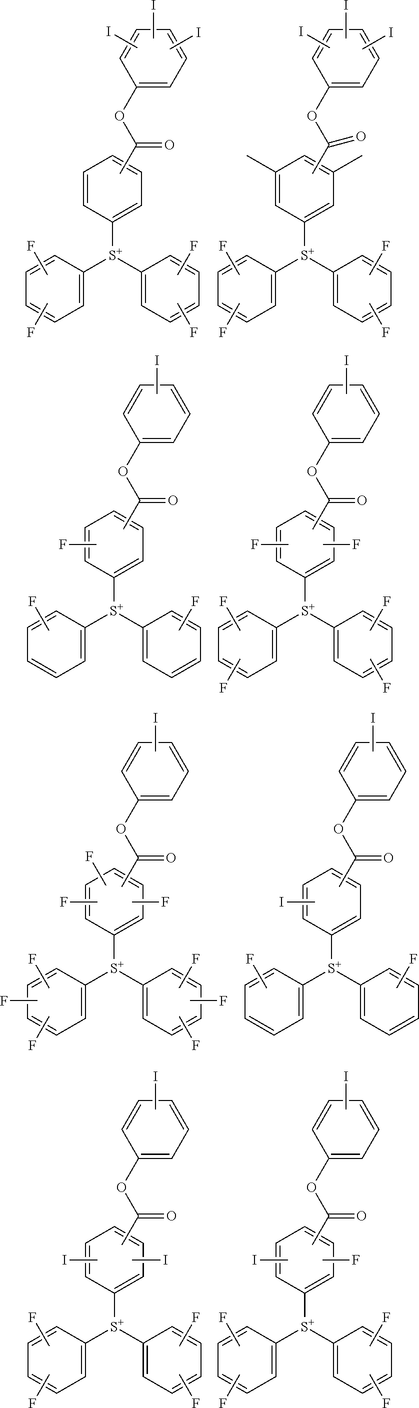

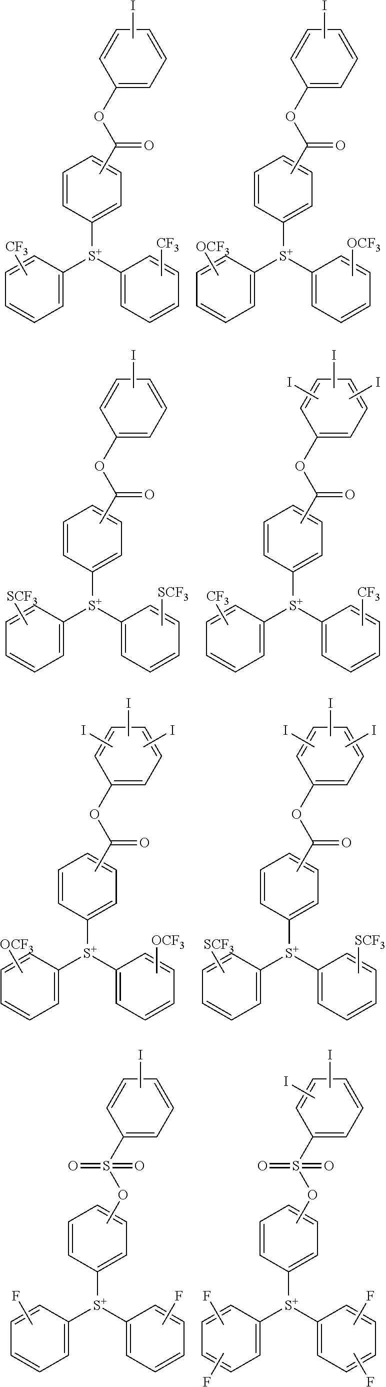

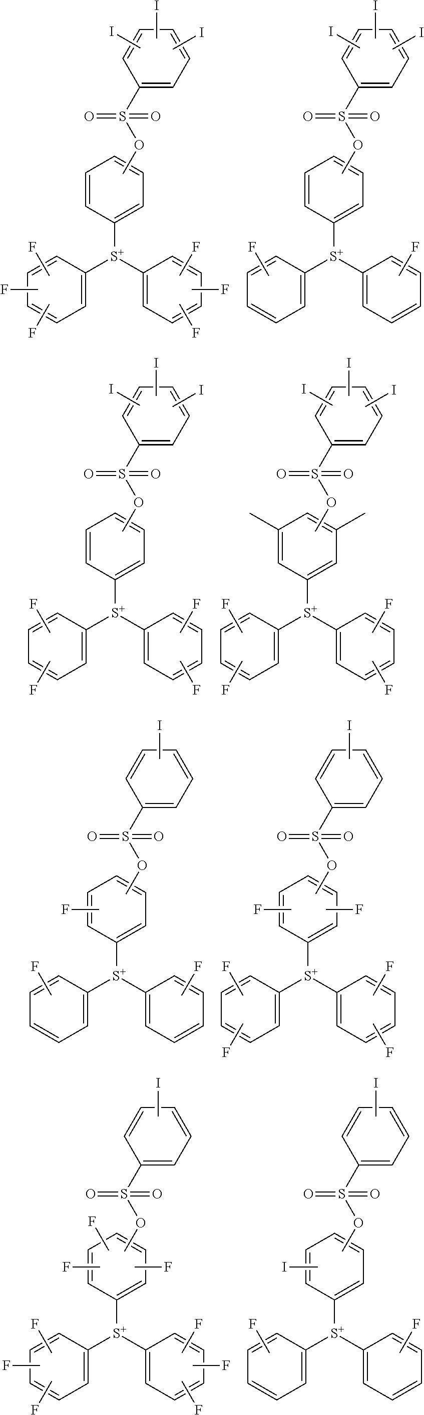

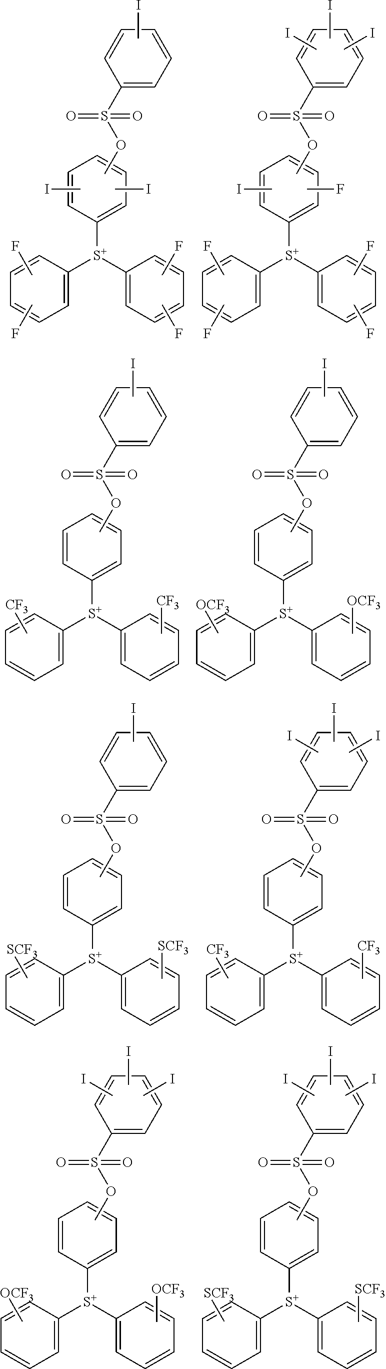

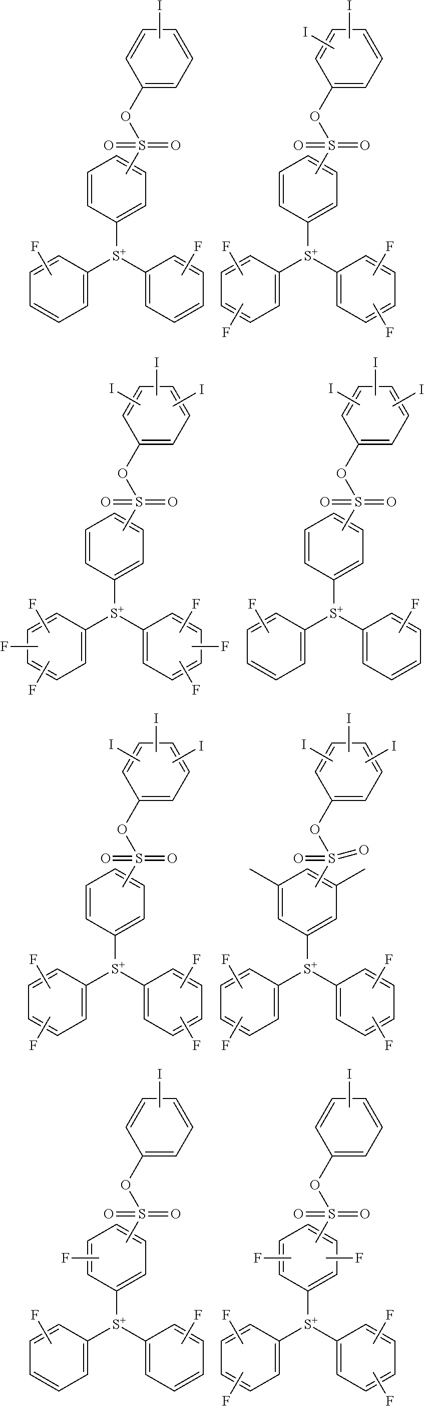

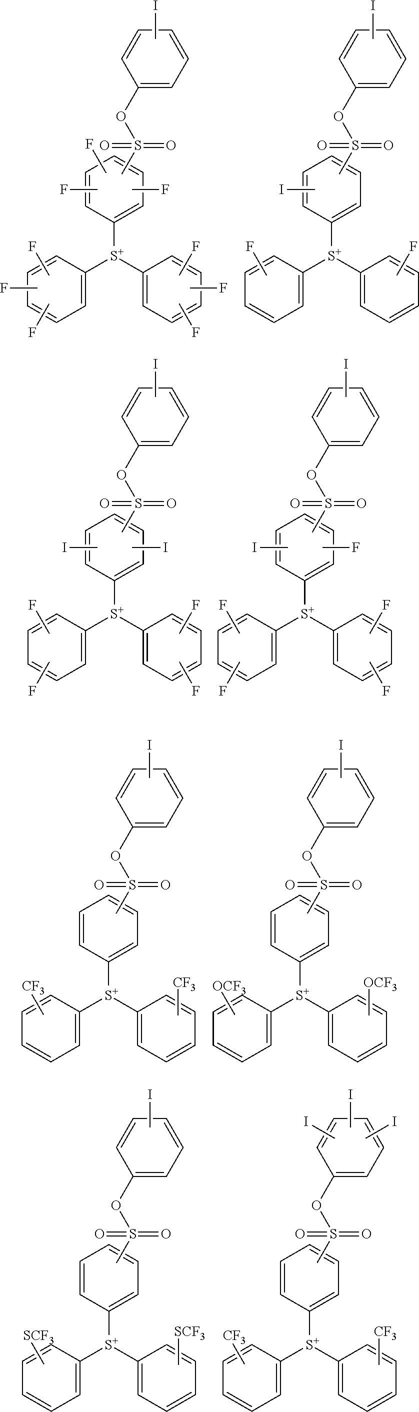

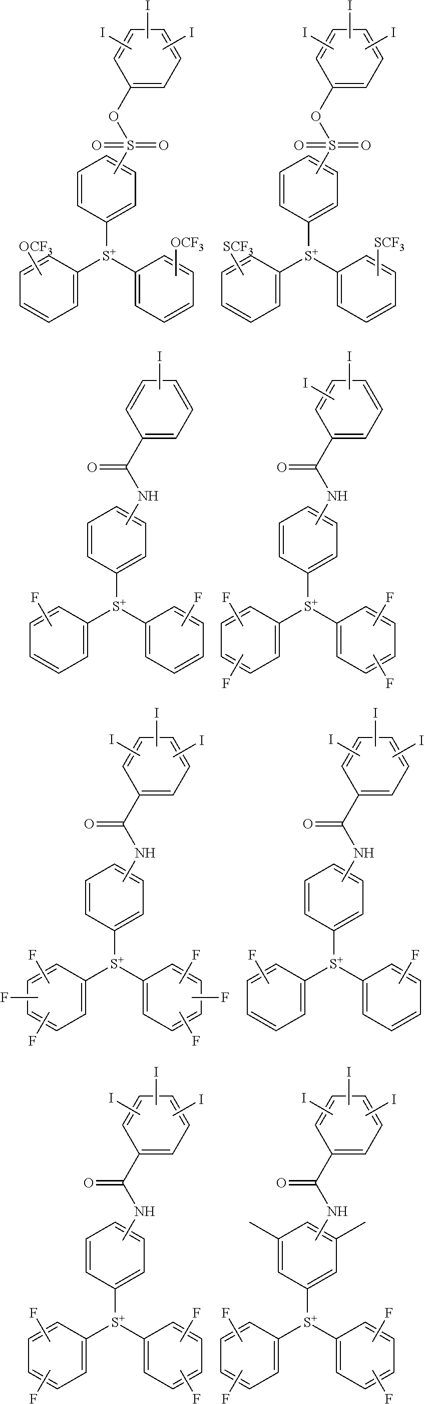

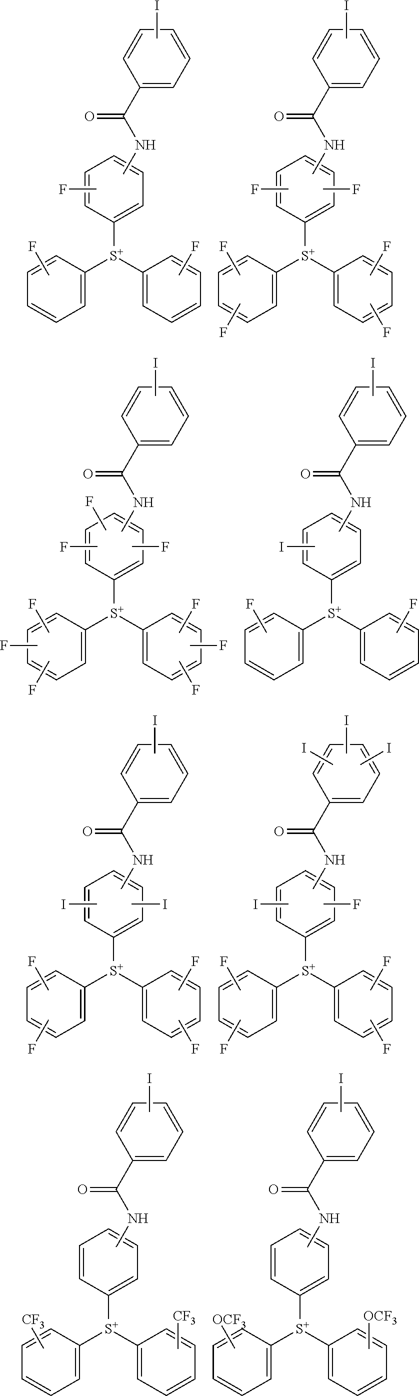

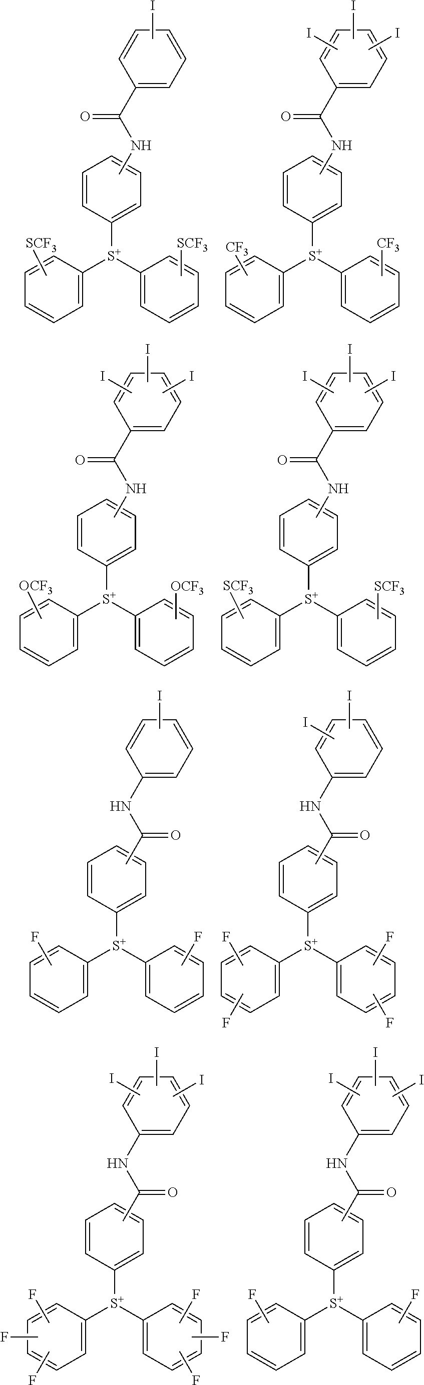

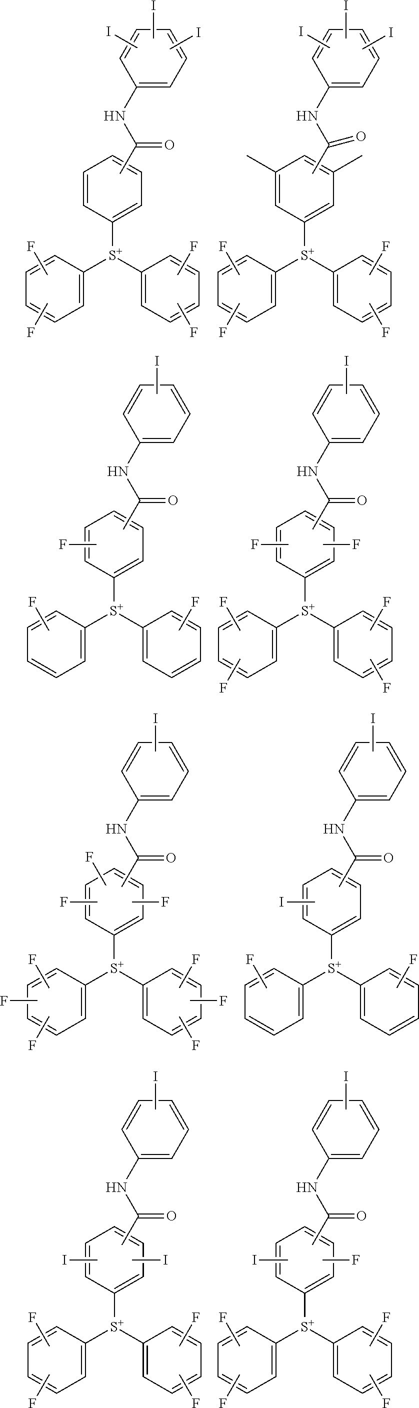

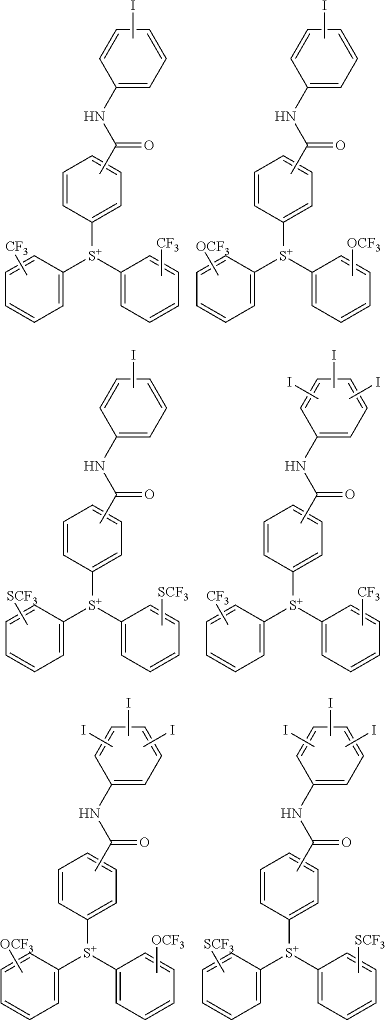

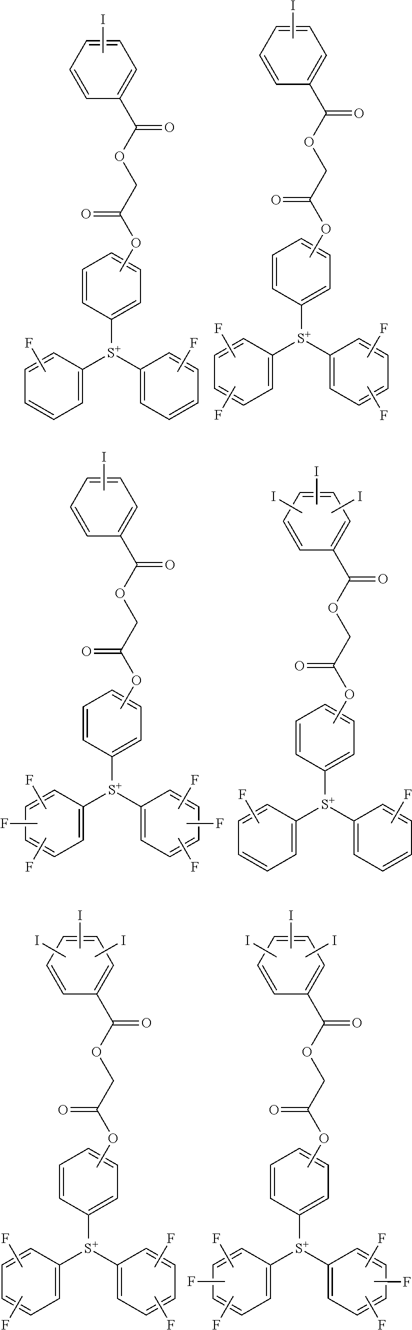

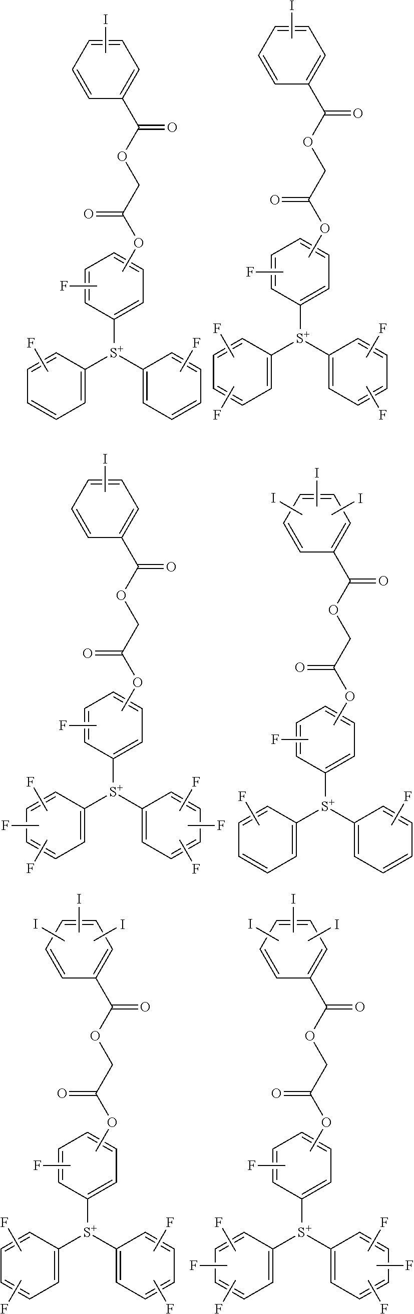

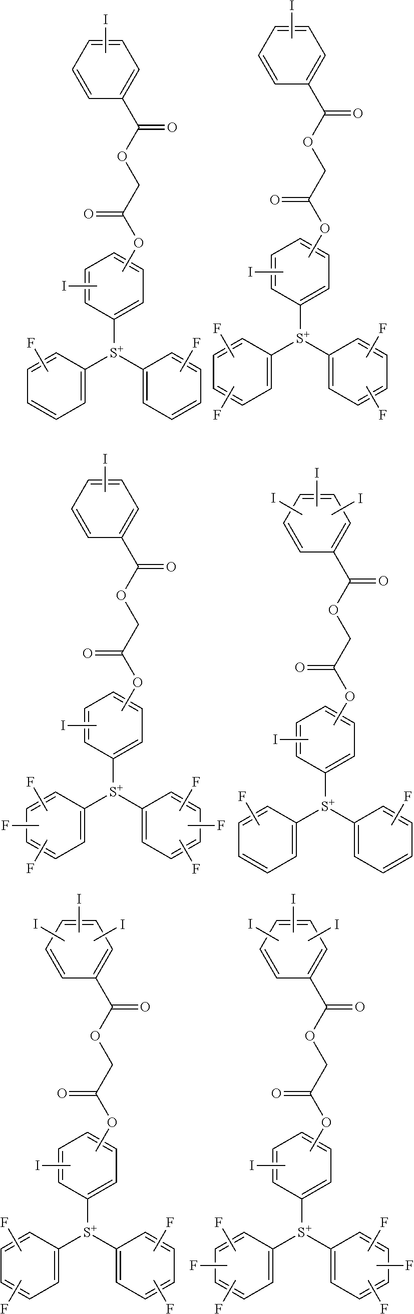

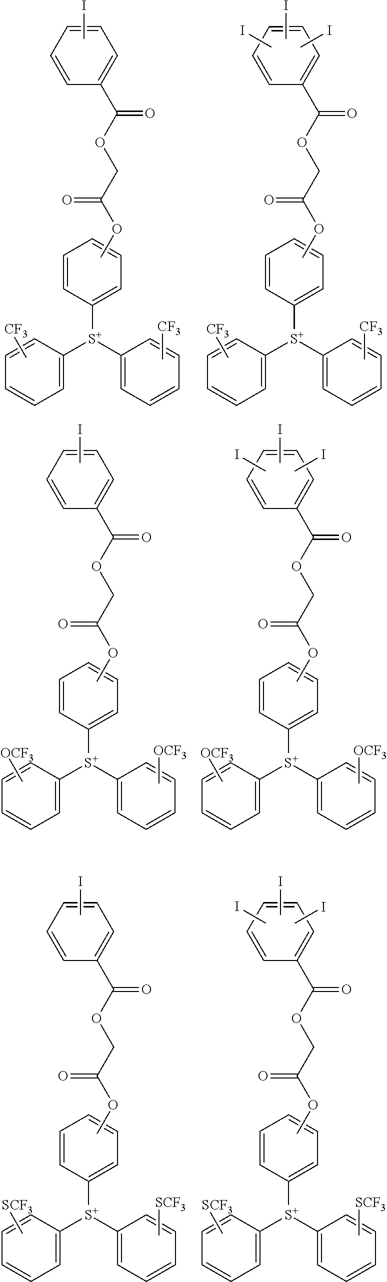

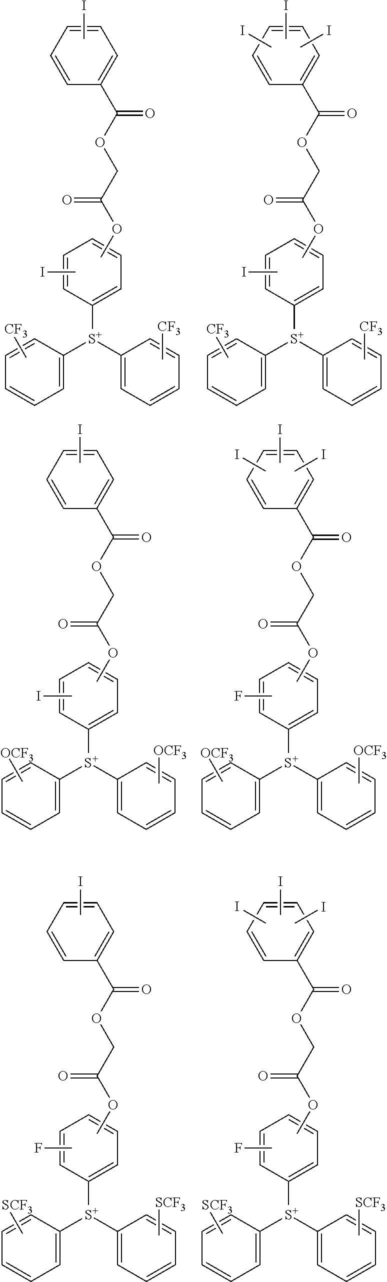

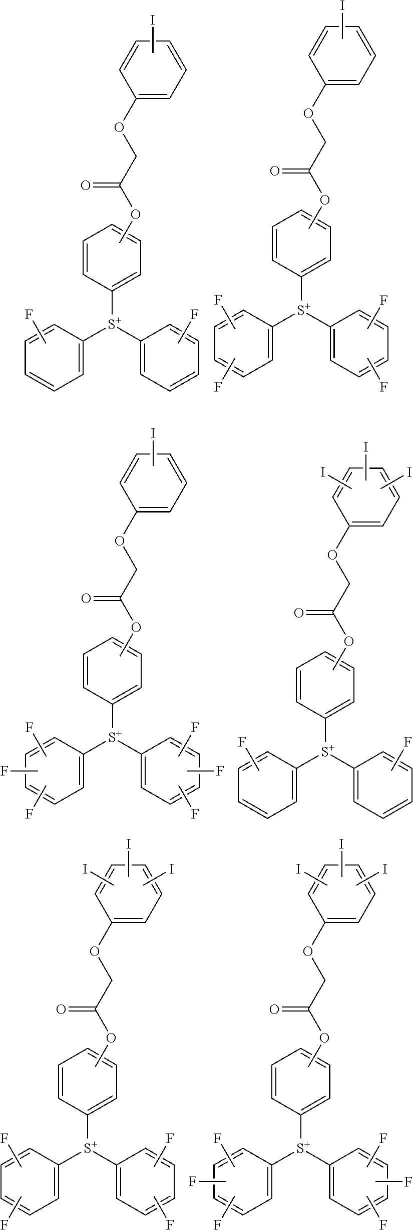

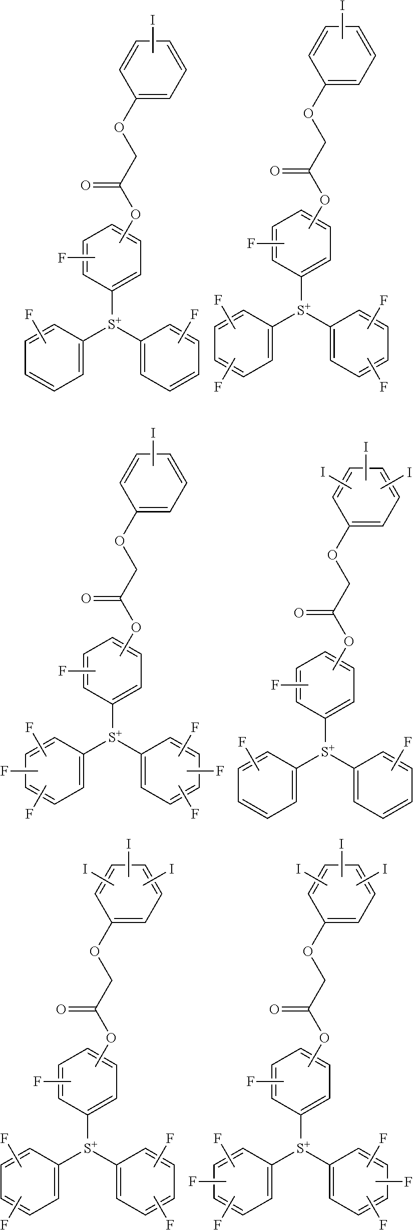

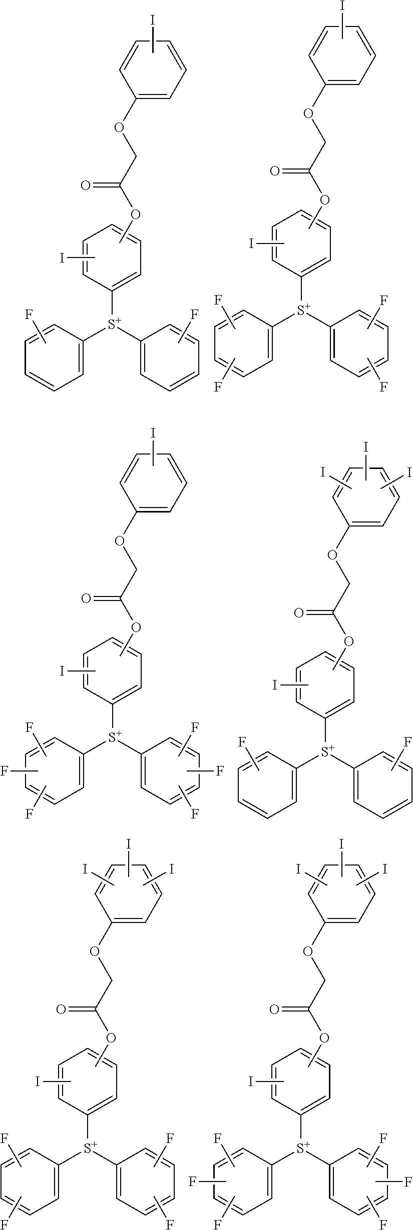

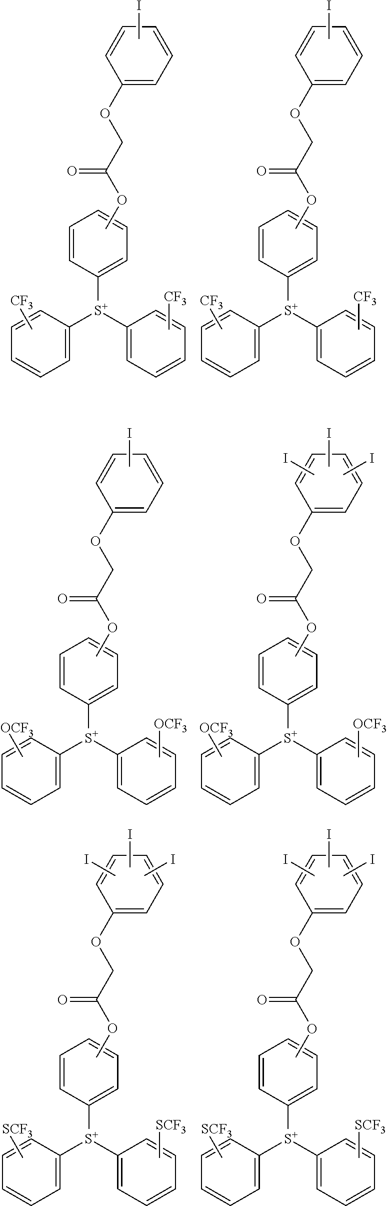

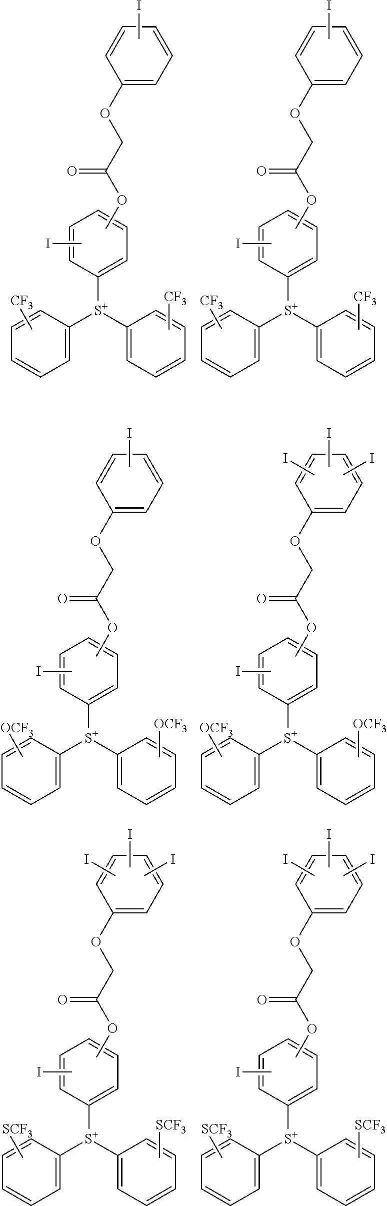

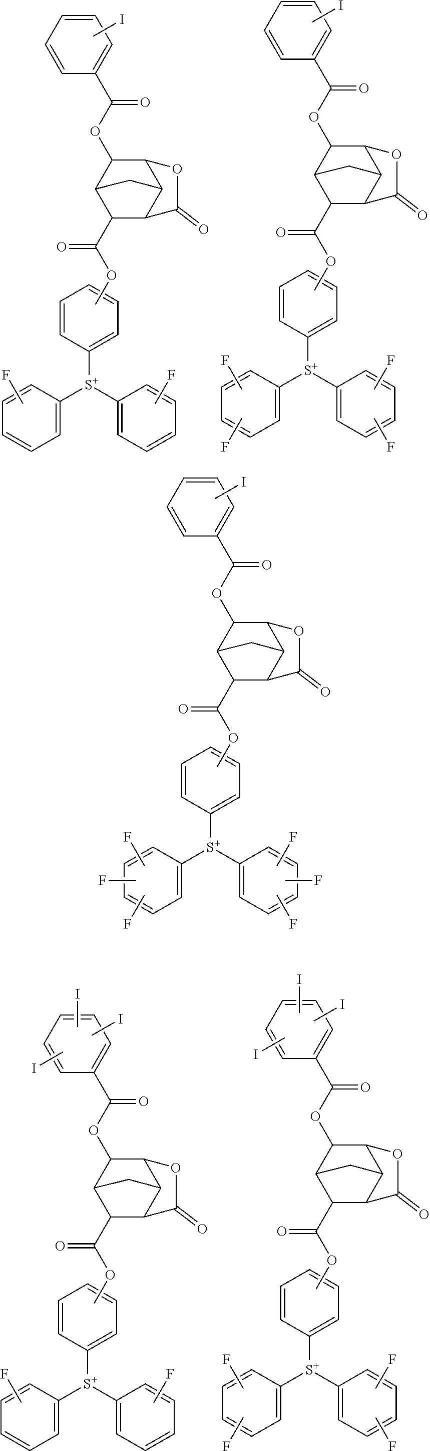

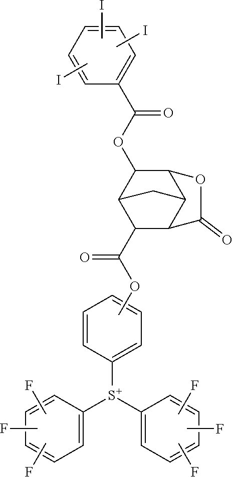

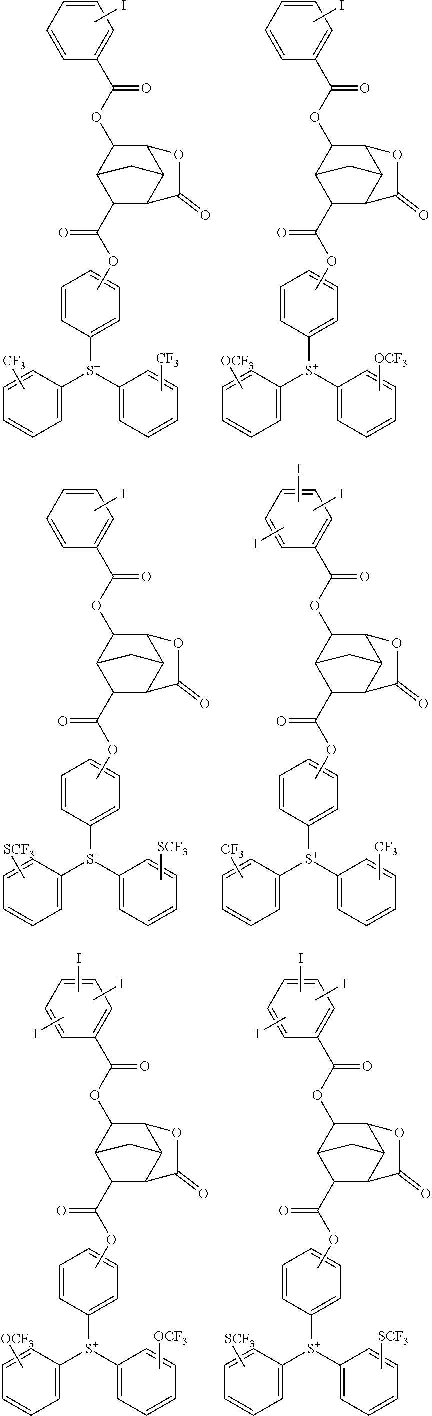

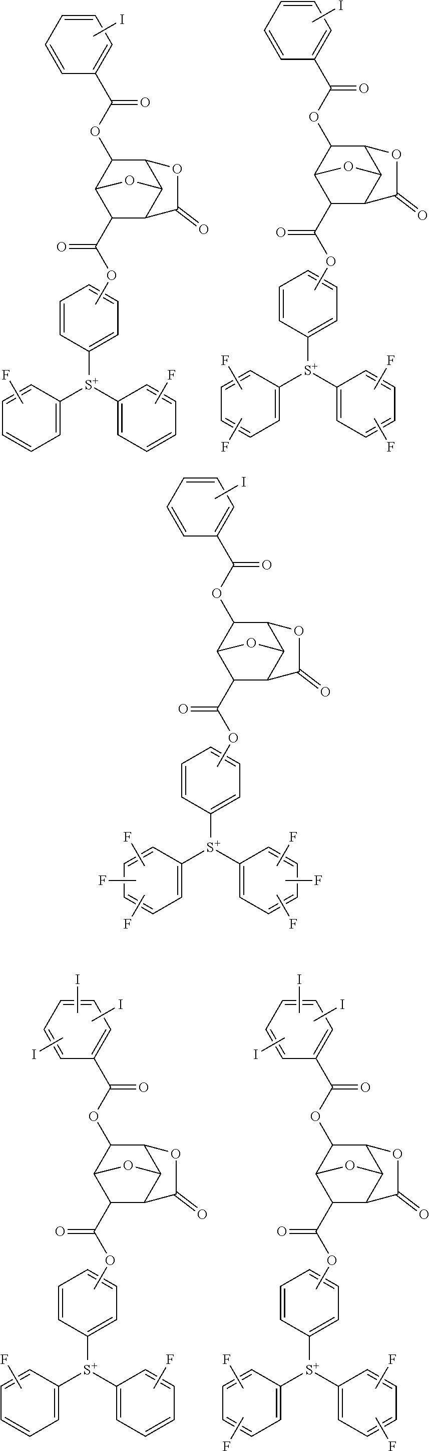

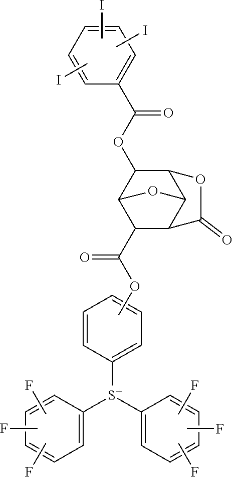

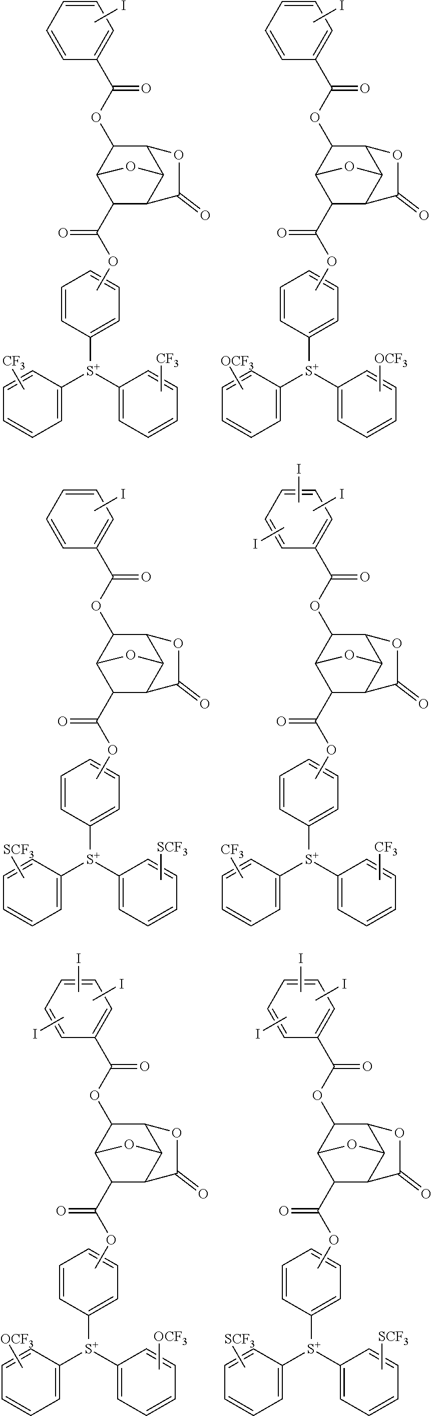

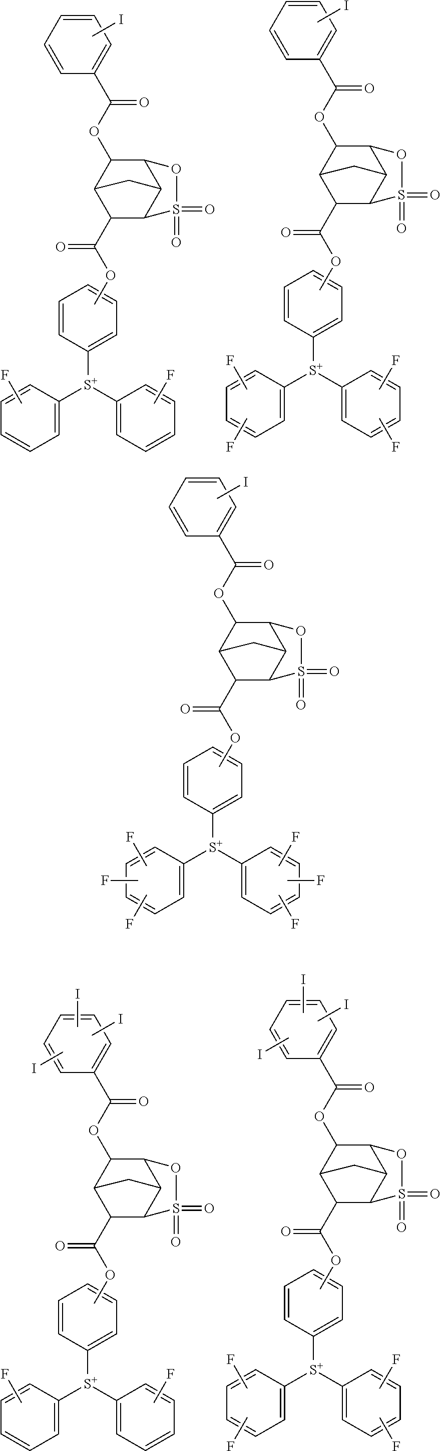

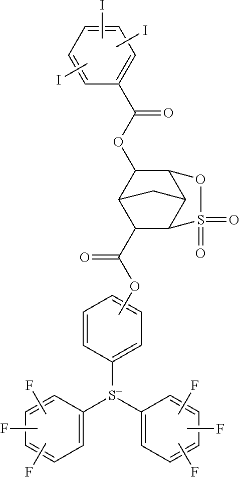

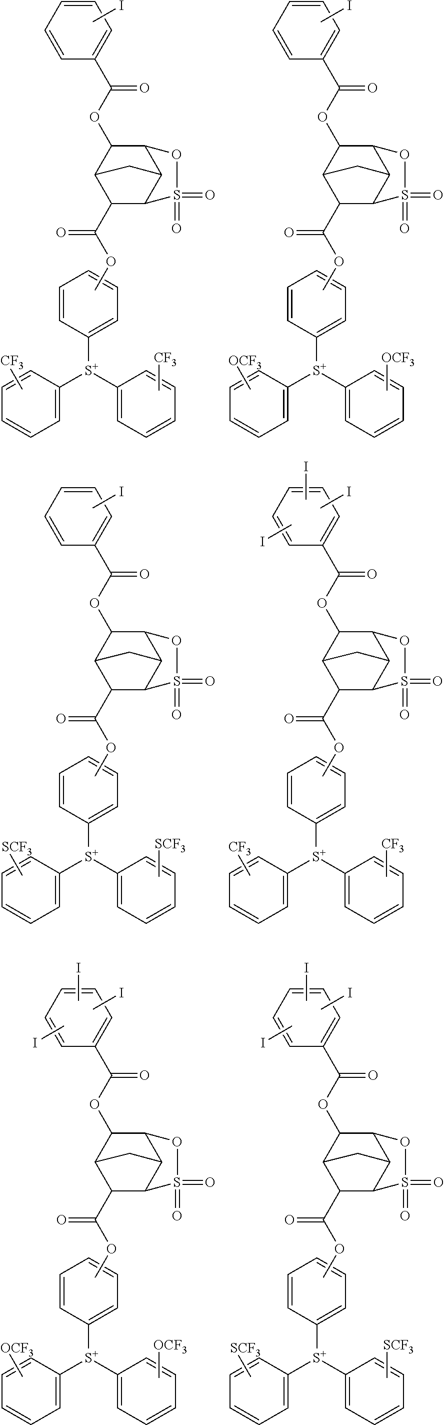

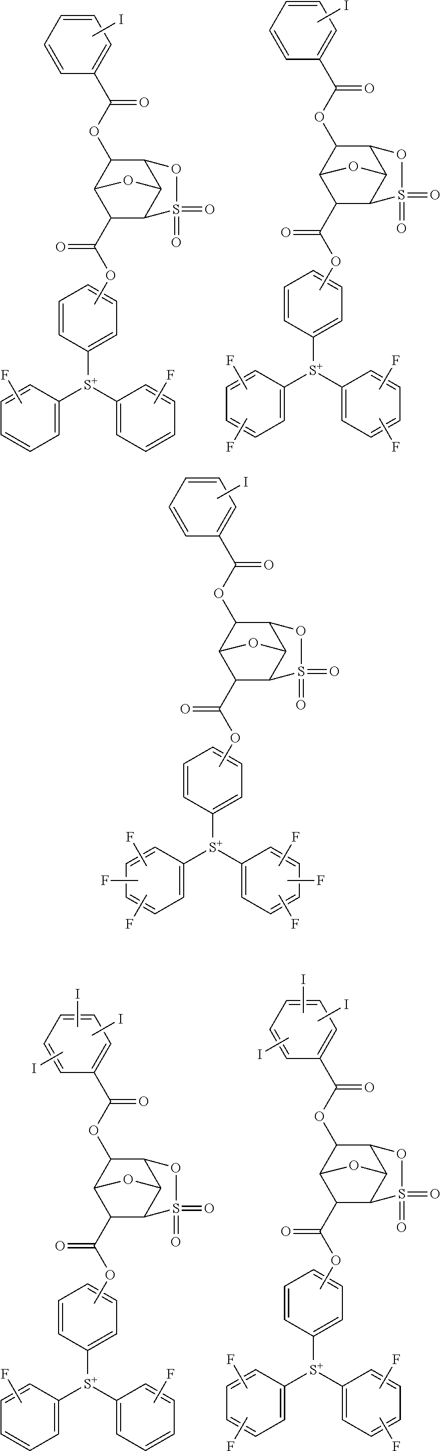

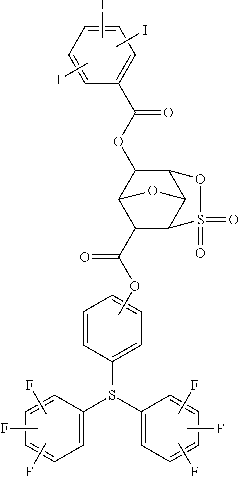

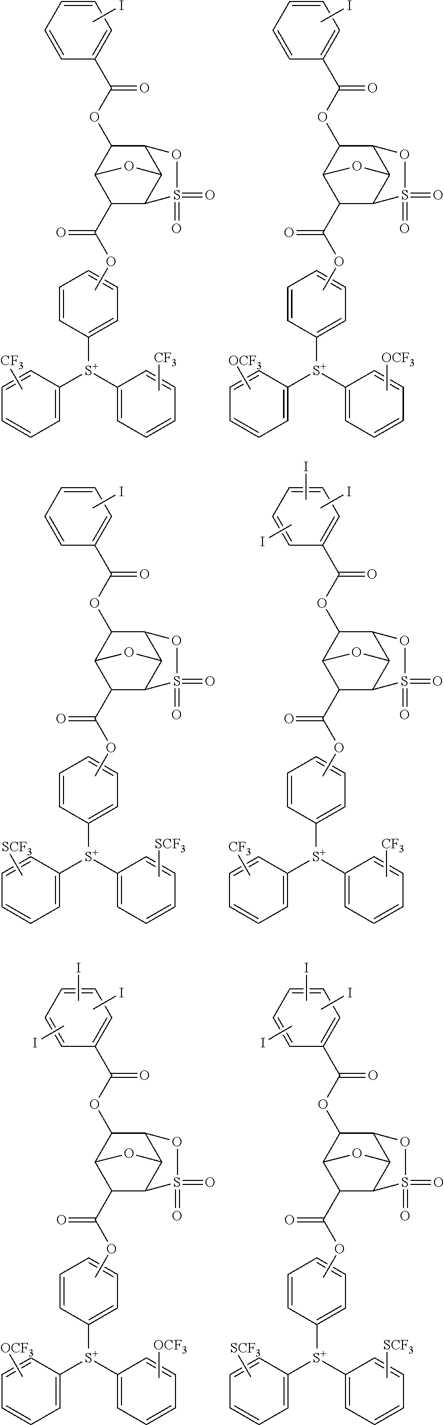

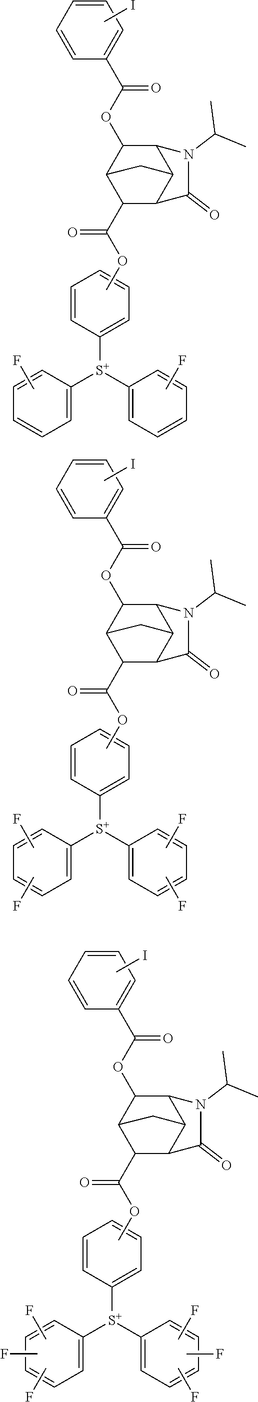

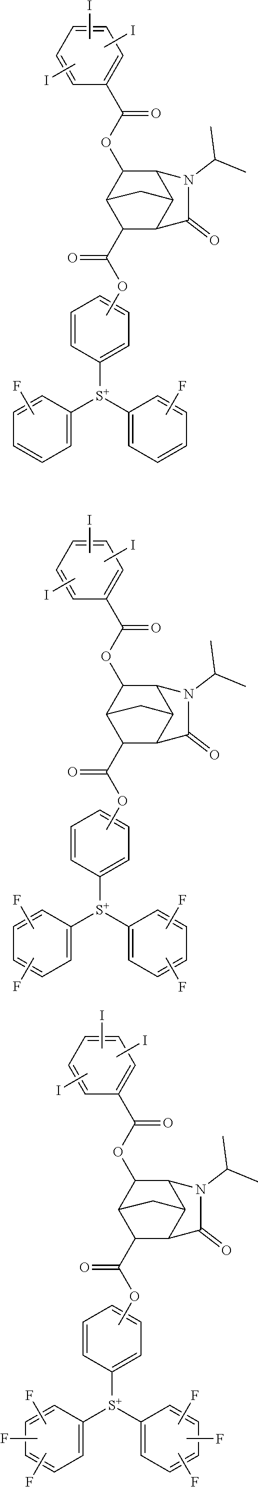

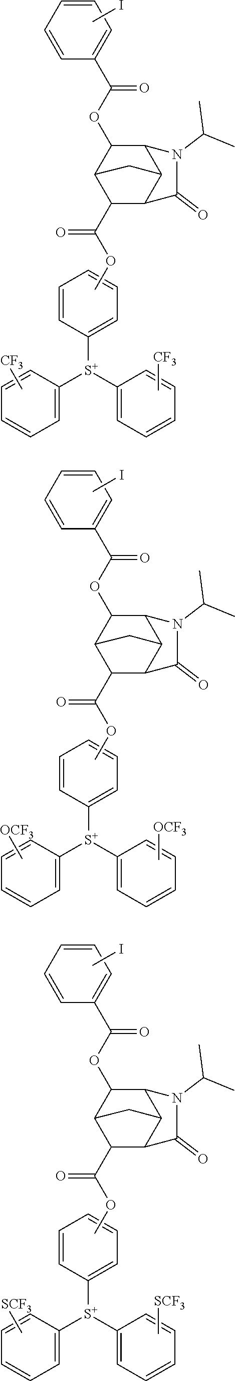

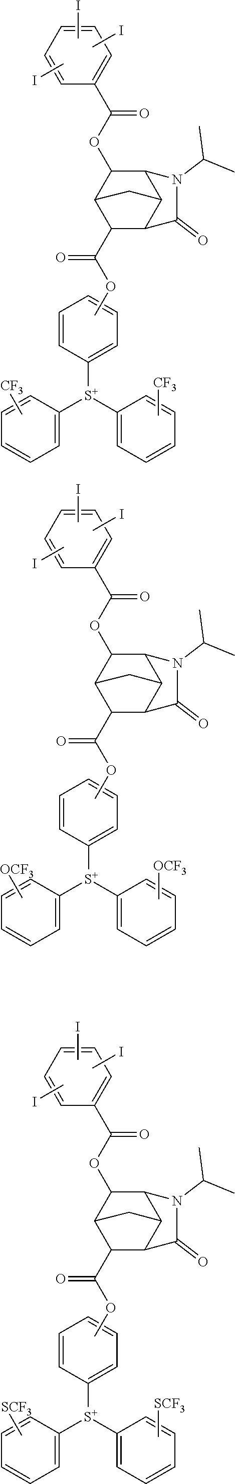

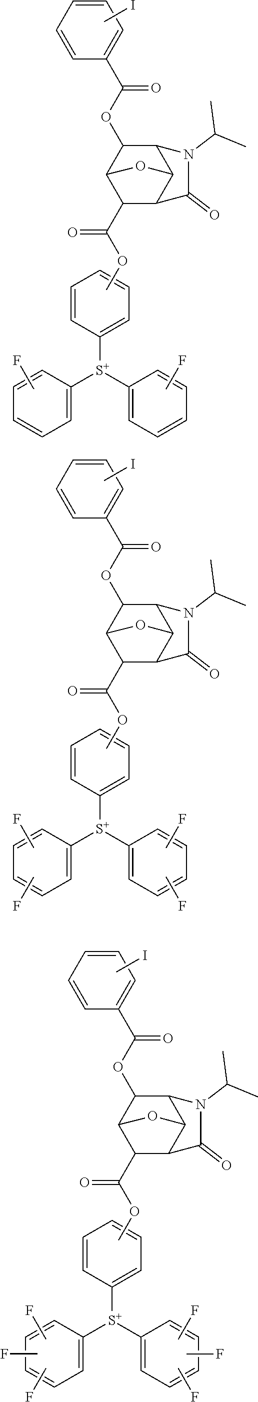

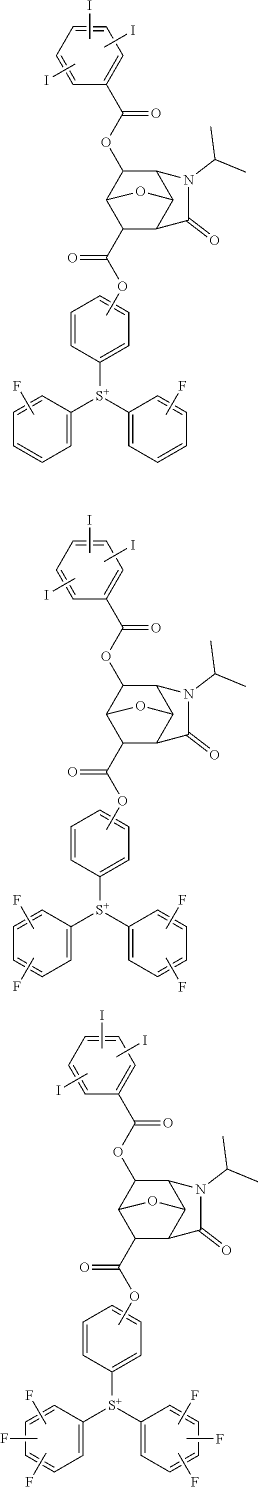

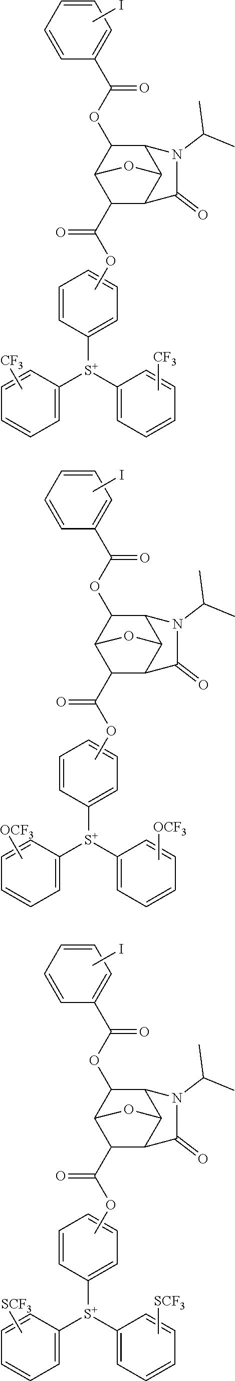

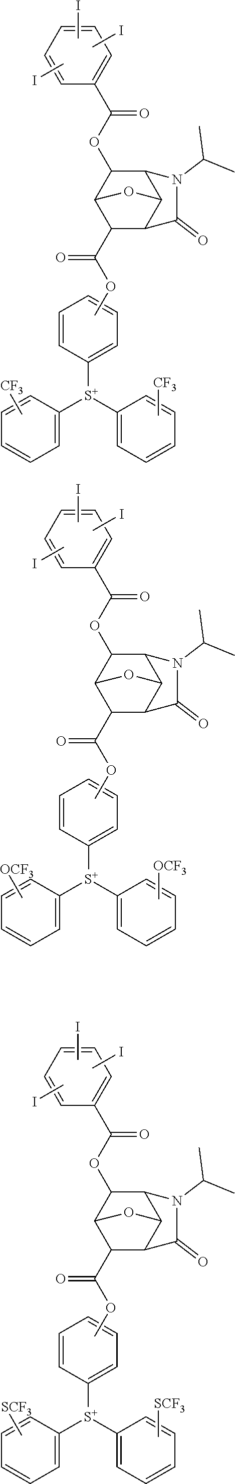

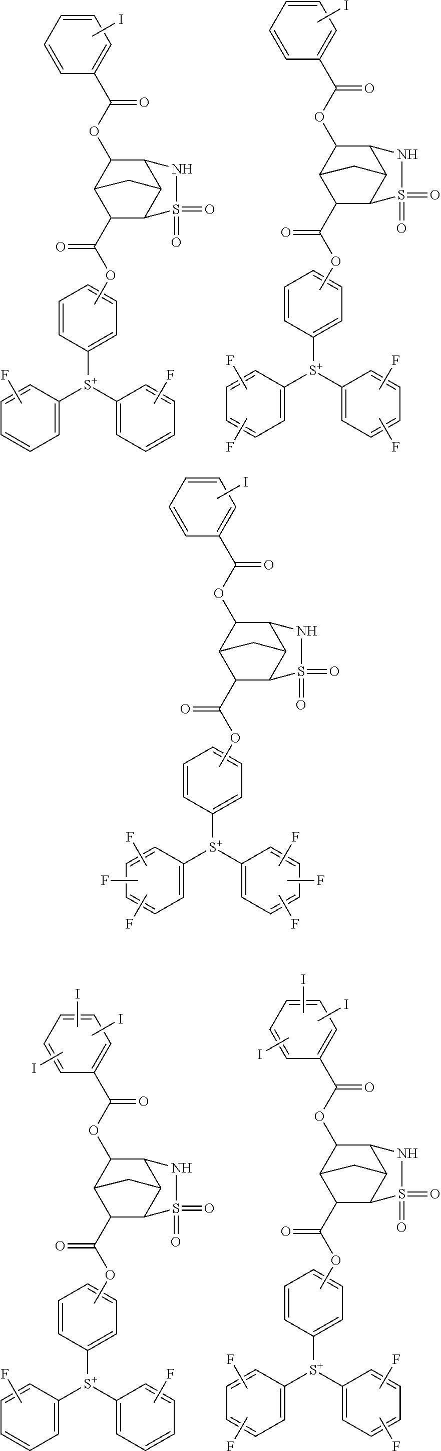

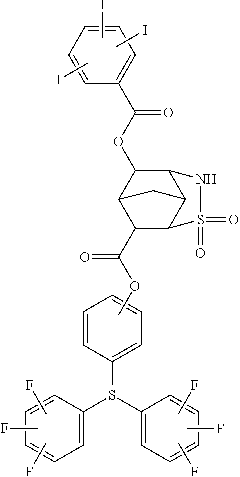

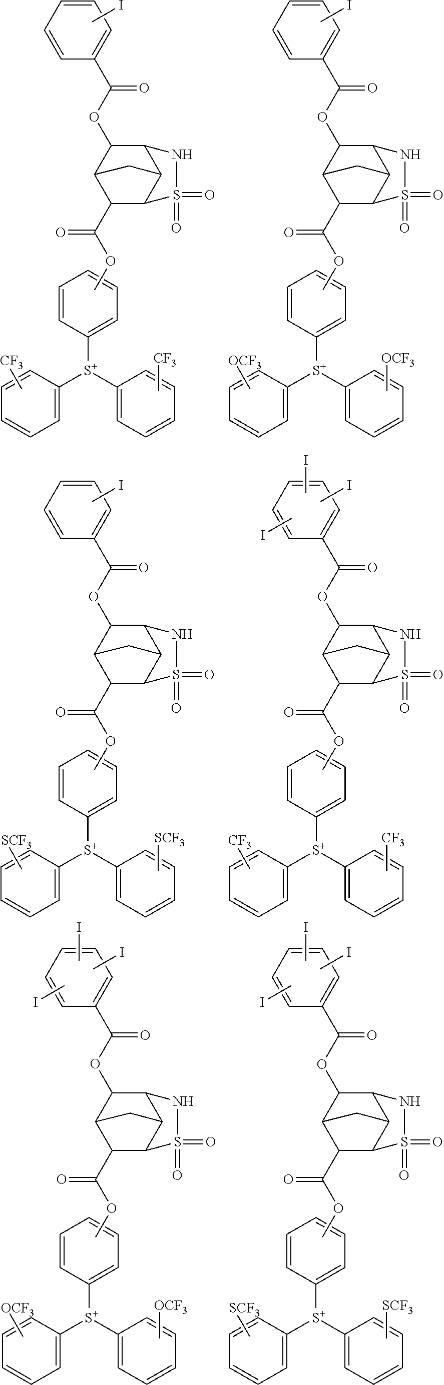

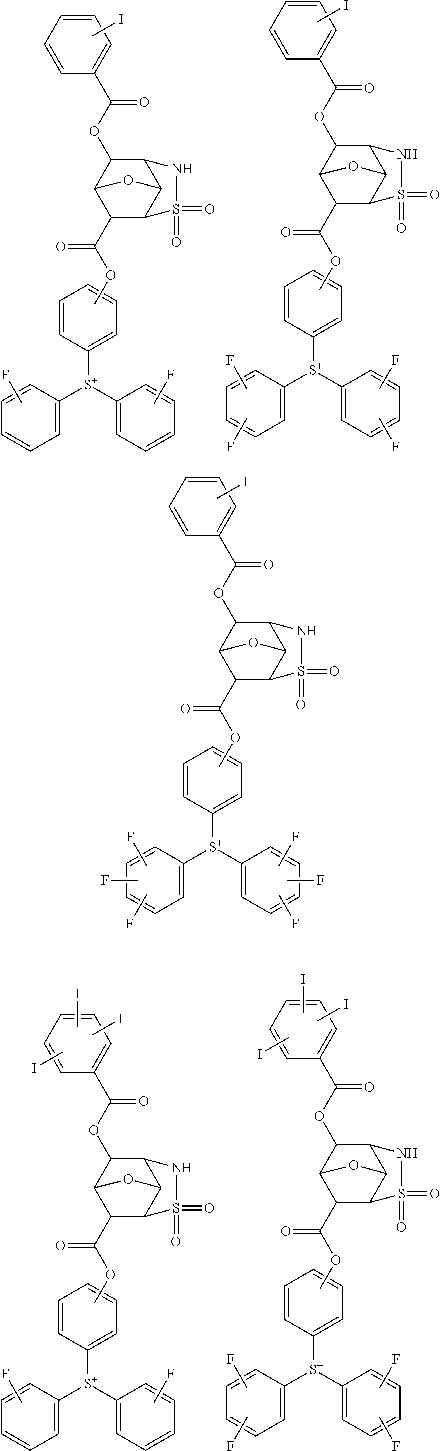

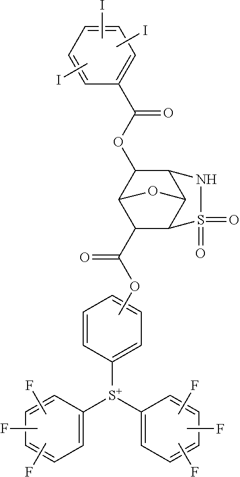

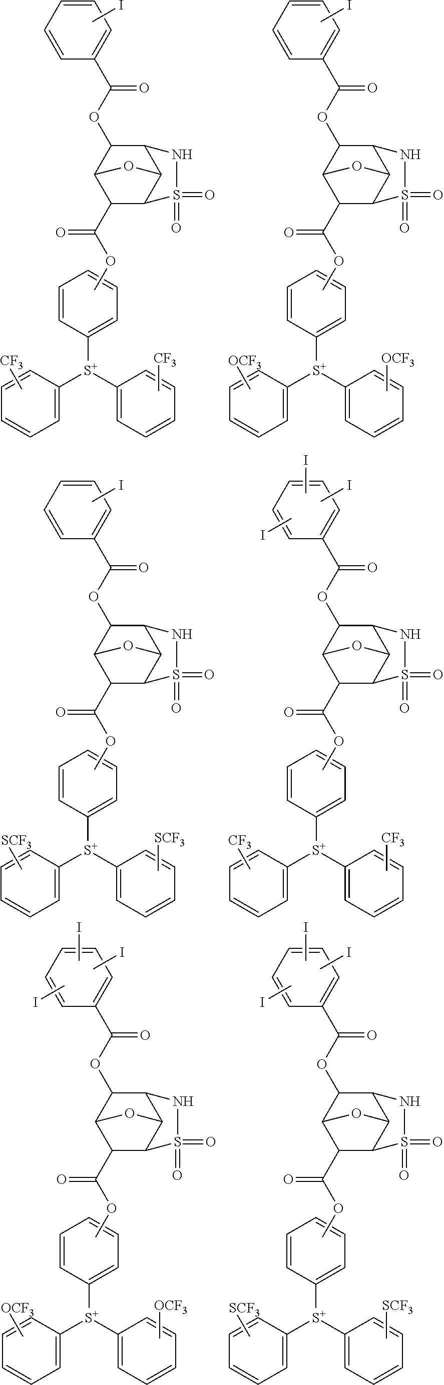

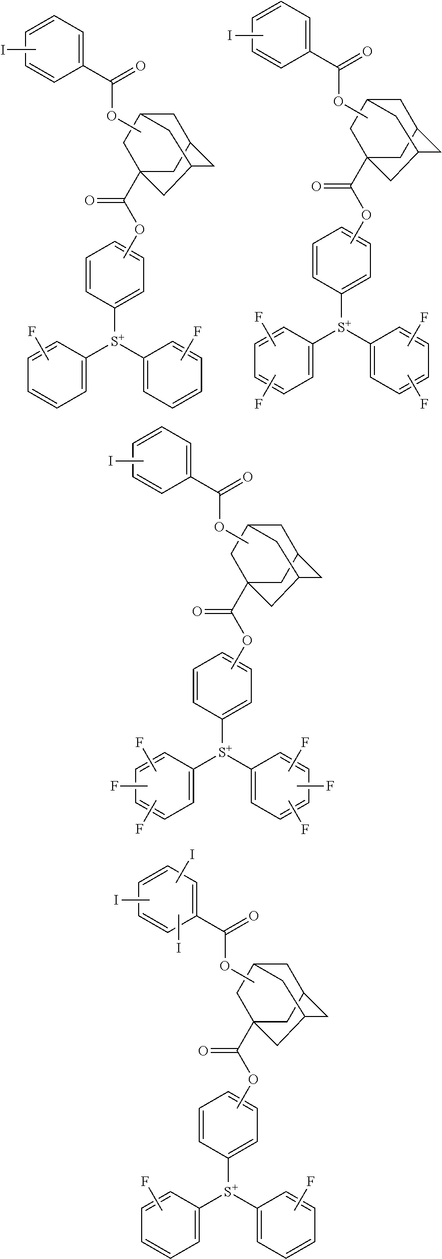

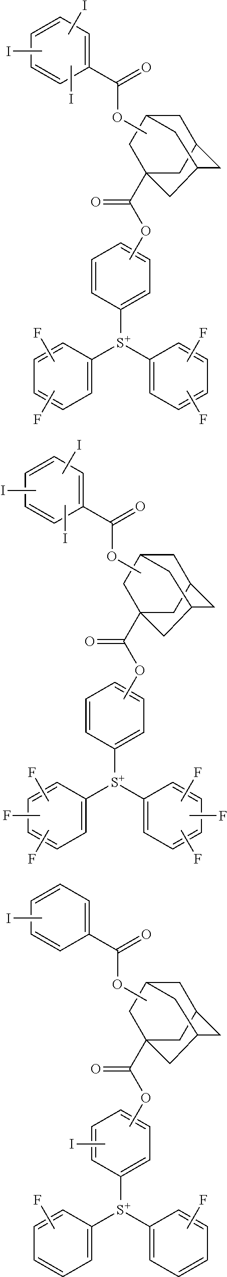

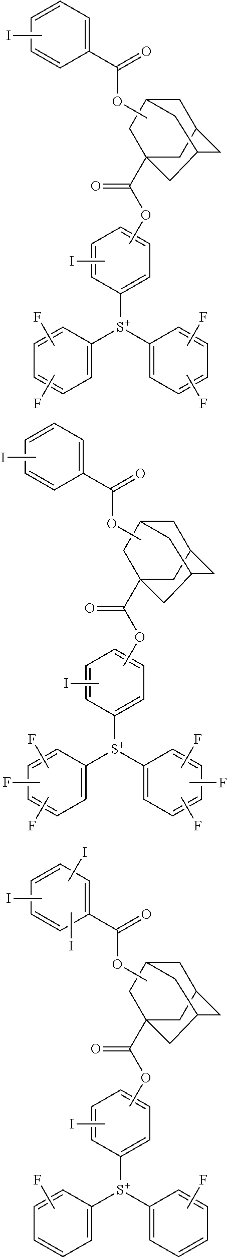

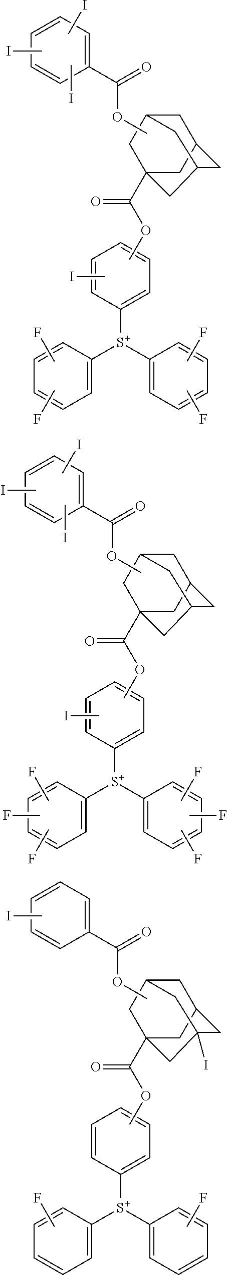

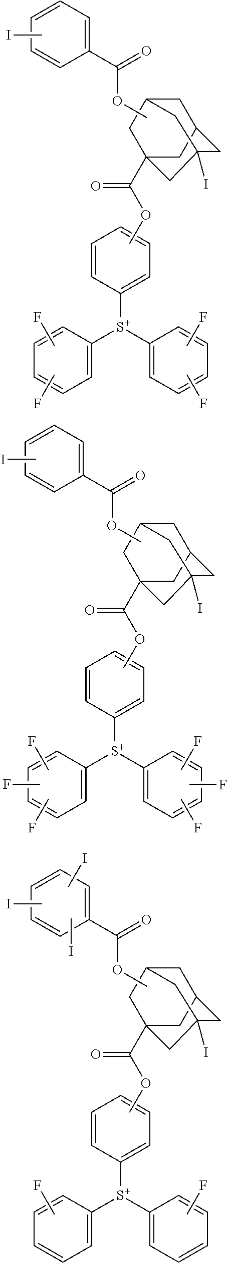

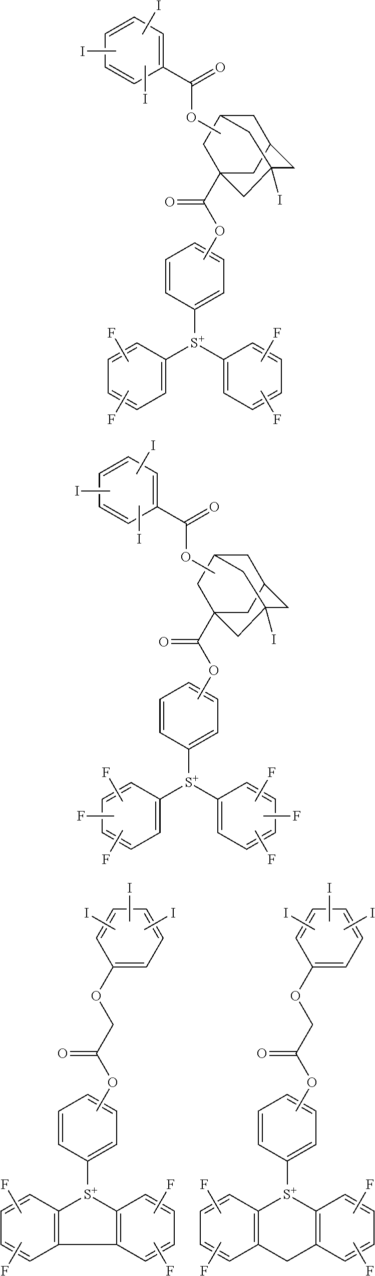

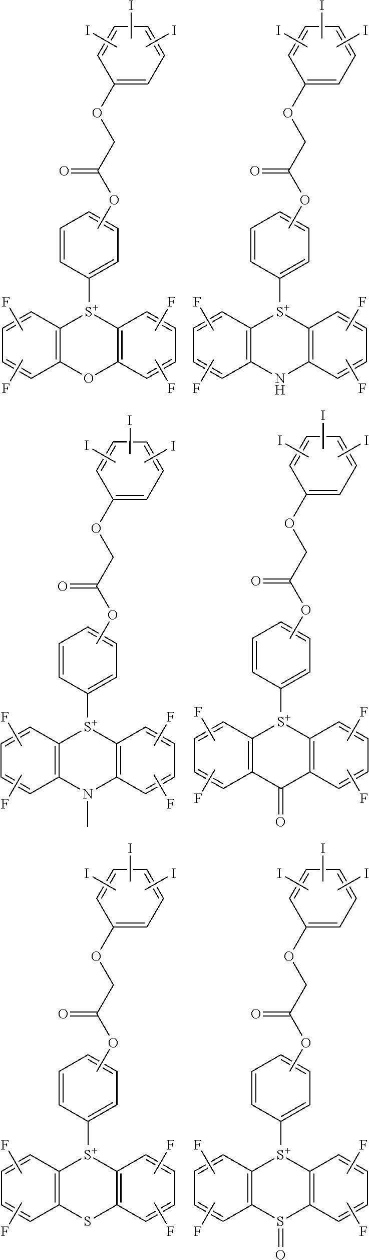

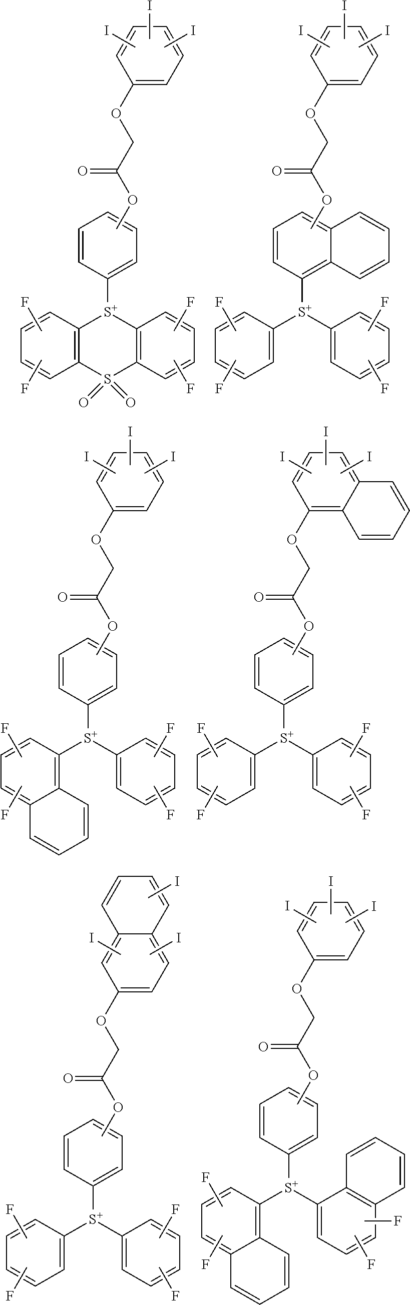

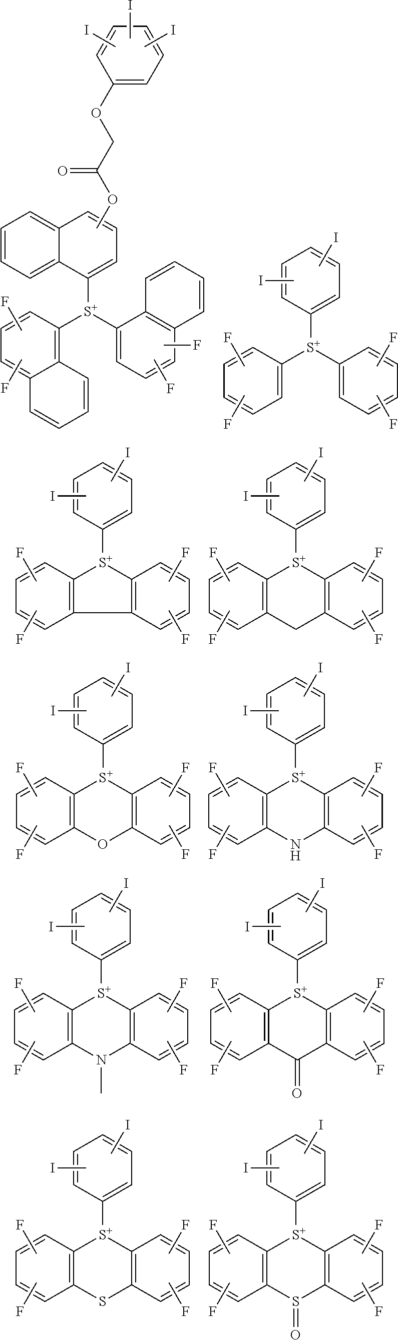

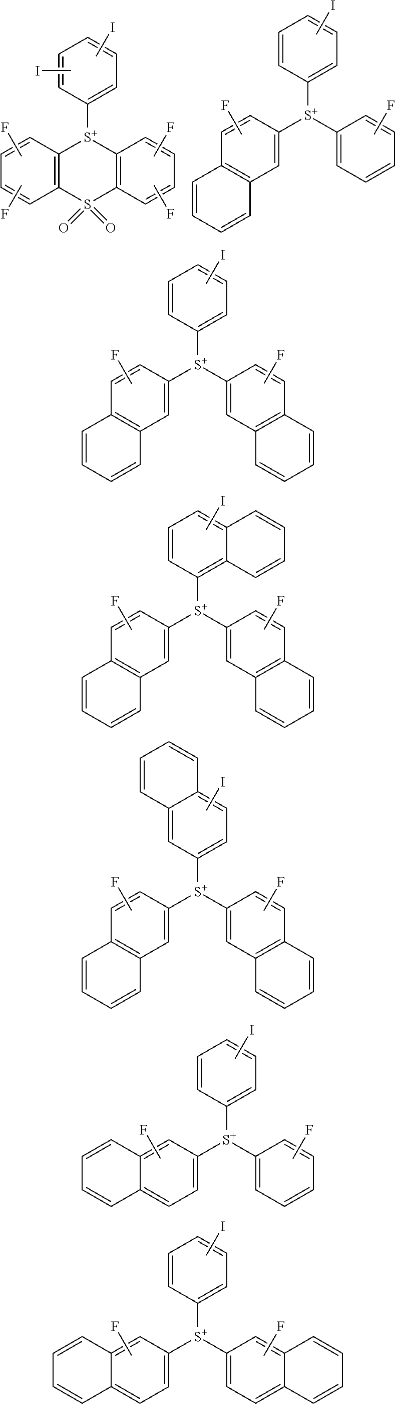

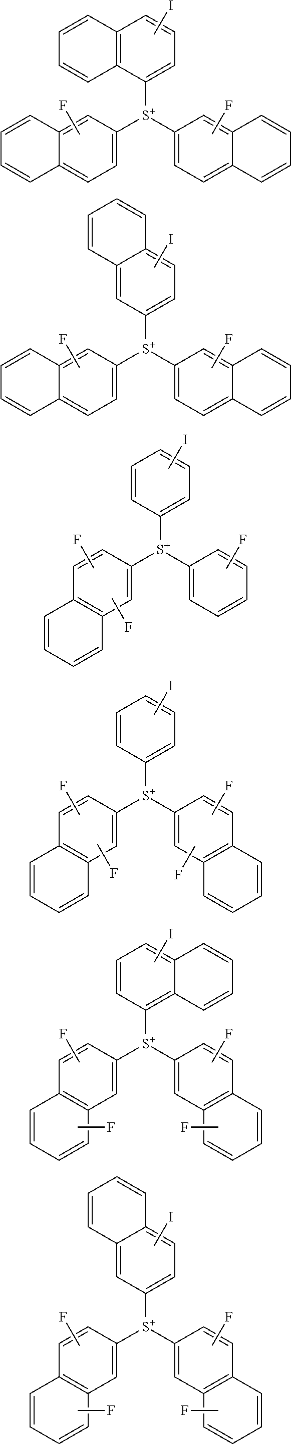

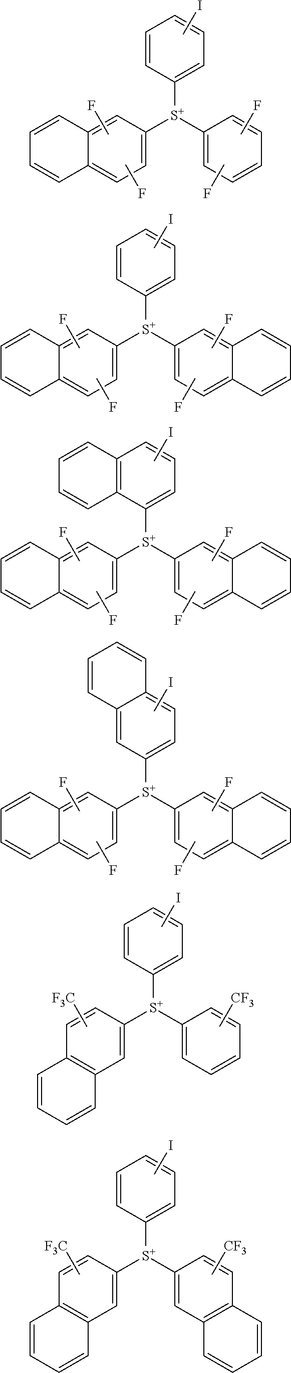

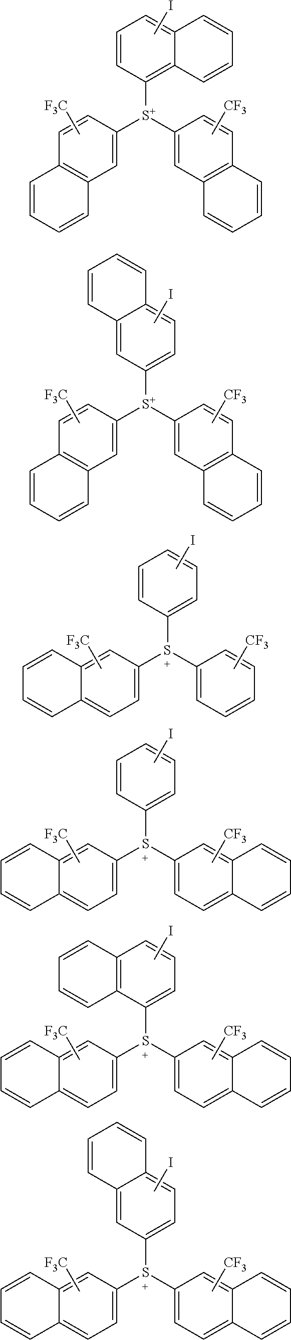

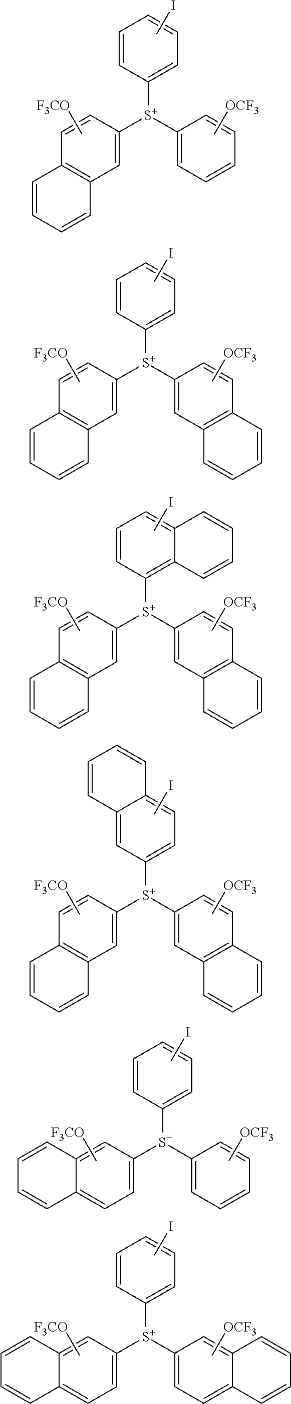

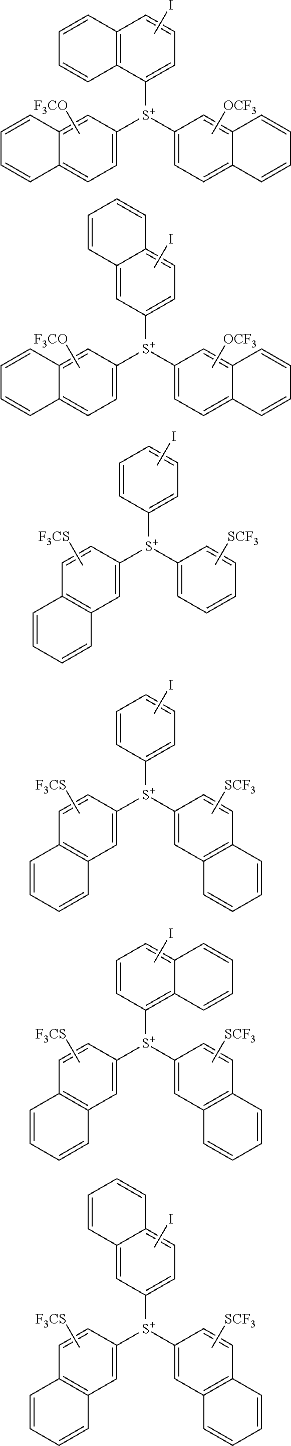

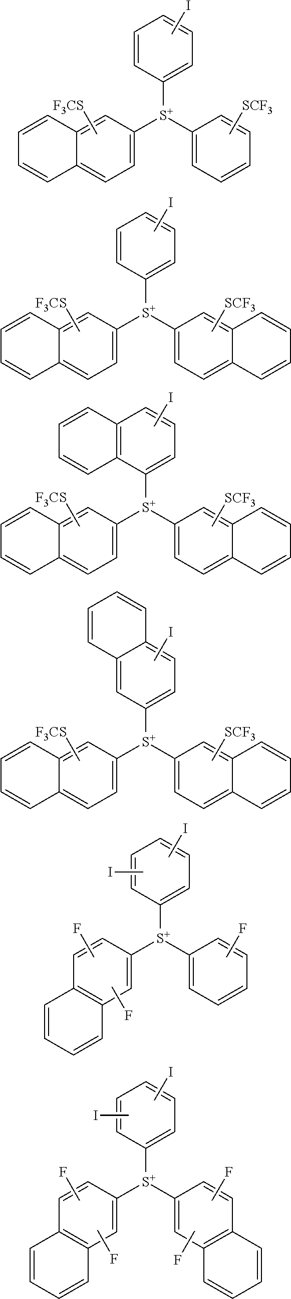

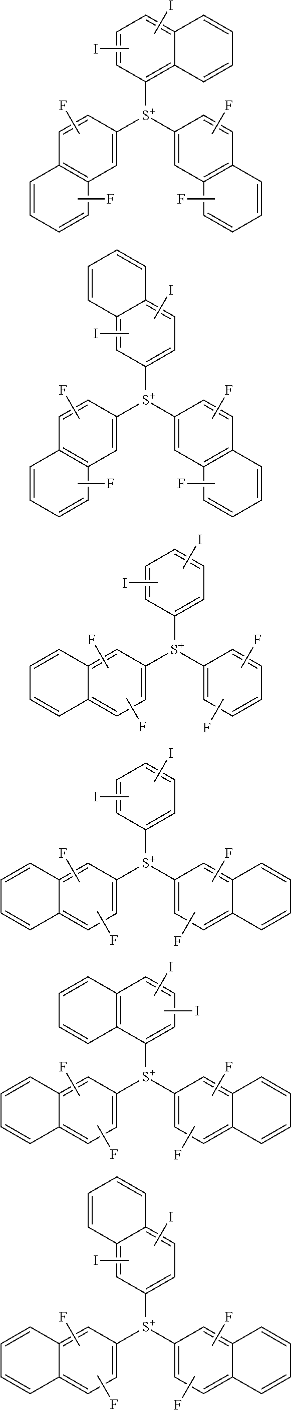

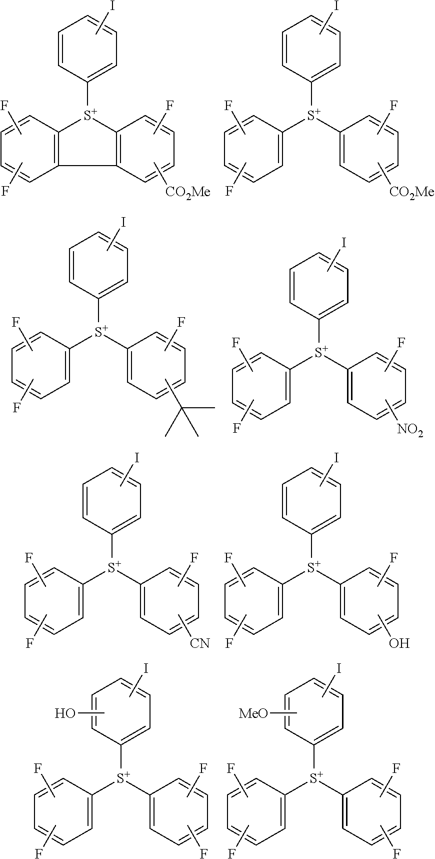

Specific examples of the onium salt of the present invention include any combination of the anion and the cation described above.

The onium salt of the present invention can be synthesized, for example, by ion-exchanging a hydrochloride or carbonate having an onium cation with a corresponding sulfoaromatic carboxylic acid derivative.

When the onium salt of the present invention and a strong acid-generating onium salt such as an α-fluorinated sulfonic acid, imide acid, or methide acid (hereinafter, these are collectively defined as a strong acid) coexist, a corresponding aromatic sulfonic acid (defined as a medium strong acid) and an aromatic carboxylic acid (defined as a weak acid) are generated from the onium salt of the present invention by light irradiation, and on the other hand, a strong acid is generated from the α-fluorinated sulfonic acid, imide acid, or methide acid. In a portion where the exposure dose is small, many onium salts that are not decomposed exist. The strong acid functions as a catalyst for causing the deprotection reaction of the base polymer, but in the weak acid and the medium strong acid generated from the onium salt of the present invention, the deprotection reaction hardly occurs. The strong acid is ion-exchanged with the remaining medium strong acid, and then ion-exchanged with a sulfonium carboxylate having the weakest acid strength to become an onium salt of the strong acid, and the carboxylic acid is released instead. In this case, when a medium strong acid exhibiting acid strength between the strong acid and the weak acid is present, proton exchange between the strong acid and the weak acid is mediated, and smooth proton exchange proceeds. When the weak acid and the medium strong acid are in the same molecule, proton transfer efficiently proceeds, which is preferable. That is, the onium salt of the present invention functions as a quencher because the strong acid is neutralized by the onium carboxylate by ion exchange. In the onium salt type quencher, LWR of a resist pattern generally tends to be smaller than that of a quencher using an amine compound. When each of the three components of the strong acid, the medium strong acid, and the weak acid is added, the proton exchange described above proceeds, but the improvement range of LWR is small since the efficiency is poor.

The salt exchange of the strong acid with the medium strong acid and the onium carboxylate is repeated indefinitely. The location where the strong acid is generated at the end of the light exposure is different from the location where the strong acid generating onium salt is present at the beginning. It is presumed that the acid generation point is averaged by repeating the cycle of acid generation and salt exchange by light many times and smoothly, whereby LWR of the resist pattern after development is reduced.

The onium salt of the present invention has an aromatic hydroxy group or an acid labile group protecting the aromatic hydroxy group in an anion as a structural feature in addition to the function as a quencher. The acid labile group in the exposed region causes a deprotection reaction by the generated strong acid to generate an aromatic hydroxy group. As a result, a contrast between the exposed region and the unexposed region is improved. In addition, it is preferable that the acid labile group is present at a position adjacent to a carbon atom to which a carboxylic acid anion is bonded, and in this case, a salicylic acid structure is formed when a deprotection reaction of the acid labile group proceeds. Salicylic acid is stabilized by a hydrogen bond between a hydroxy group and a carboxy group, and reduces swelling with respect to an alkaline developer in the exposed region. As a result, collapse of the resist pattern generated in the unexposed region is suppressed. In addition, in the exposed region, a sulfonic acid is also generated in the same molecule, and three polar groups of a sulfo group, a carboxy group, and a hydroxy group are generated, such that affinity to an alkaline developer is increased, and the sulfonic acid is effectively removed while swelling is suppressed, thereby suppressing development defects. Due to these synergistic effects, when the onium salt of the present invention is used, the dissolution contrast is high, LWR of the line pattern and CDU of the hole pattern are excellent, and pattern formation resistant to pattern collapse becomes possible.

### Chemically Amplified Resist Composition

The chemically amplified resist composition of the present invention comprises (A) a quencher consisting of an onium salt having formula (1).

In the chemically amplified resist composition of the present invention, the content of the quencher consisting of the onium salt having formula (1) as the component (A) is preferably 0.001 to 30 parts by weight, and more preferably 0.01 to 20 parts by weight per 80 parts by weight of a base polymer to be described below. The quencher (A) may be used alone or in admixture.

### (B) Base Polymer

The chemically amplified resist composition of the present invention may comprise a base polymer as a component (B). Examples of the base polymer (B) include a base polymer comprising repeat units having the formula (a1) (hereinafter, also referred to as repeat units a1) or repeat units having the formula (a2) (hereinafter, also referred to as repeat units a2).

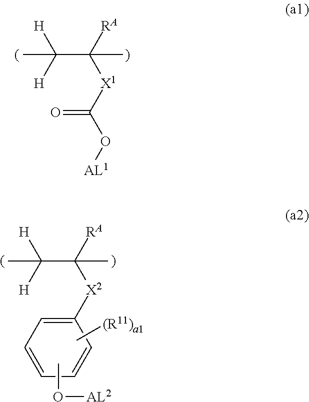

In formulae (a1) and (a2), R^{A} is each independently a hydrogen atom, a fluorine atom, a methyl group, or a trifluoromethyl group.

In formula (a1), X¹ is a single bond, a phenylene group, a naphthylene group, *—C(═O)—O—X¹¹—, or *—C(═O)—NH—X¹¹—, and the phenylene group or the naphthylene group may be substituted by a hydroxy group, a nitro group, a cyano group, an optionally fluorinated C₁-C₁₀ saturated hydrocarbyl group, an optionally fluorinated C₁-C₁₀ saturated hydrocarbyloxy group, or a halogen atom. X¹¹ is a C₁-C₁₀ saturated hydrocarbylene group, a phenylene group, or a naphthylene group which may contain a hydroxy group, an ether bond, an ester bond, or a lactone ring. * designates a point of attachment to the carbon atom in the backbone.

In formula (a2), X² is a single bond, *—C(═O)—O—, or *—C(═O)—NH—. * designates a point of attachment to the carbon atom in the backbone. R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. a1 is 0, 1, 2, 3, or 4, and preferably 0 or 1.

In formulae (a1) and (a2), A^{L1} and A^{L2} are each independently an acid labile group. Specific examples of the acid labile group include those described in JP-A 2013-80033 and JP-A 2013-83821.

Typically, specific examples of the acid labile group include those having the formulae (AL-3) to (AL-5).

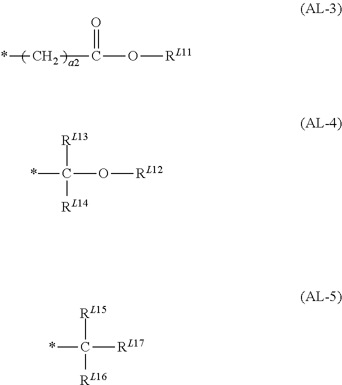

Herein the broken line designates a point of attachment.

In formulae (AL-3) and (AL-4), R^{L11} and R^{L12} are each independently a C₁-C₄₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyl group.

In formula (AL-3), a2 is an integer of 0 to 10, and is preferably 1, 2, 3, 4 or 5.

In formula (AL-4), R^{L13} and R^{L14} are each independently a hydrogen atom or a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyl group. Any two of R^{L12}, R^{L13}, and R^{L14} may bond together to form a C₃-C₂₀ ring with the carbon atom or the carbon atom and the oxygen atom to which they are attached. The ring is preferably a C₄-C₁₆ ring, and particularly preferably an alicyclic ring. In formula (AL-5), R^{L15}, R^{L16}, and R^{L17} are each independently a C₁-C₂₀ hydrocarbyl group, and may contain a heteroatom such as an oxygen atom, a sulfur atom, a nitrogen atom, or a fluorine atom. The hydrocarbyl group may be saturated or unsaturated and straight, branched, or cyclic. The hydrocarbyl group is preferably a C₁-C₂₀ hydrocarbyl group. Any two of R^{L15}, R^{L16}, and R^{L17} may bond together to form a C₃-C₂₀ ring with the carbon atom to which they are attached. The ring is preferably a C₄-C₁₆ ring, and particularly preferably an alicyclic ring.

Specific examples of the repeat units a1 are shown below, but are not limited thereto. Herein R^{A} and AL¹ are as defined above.

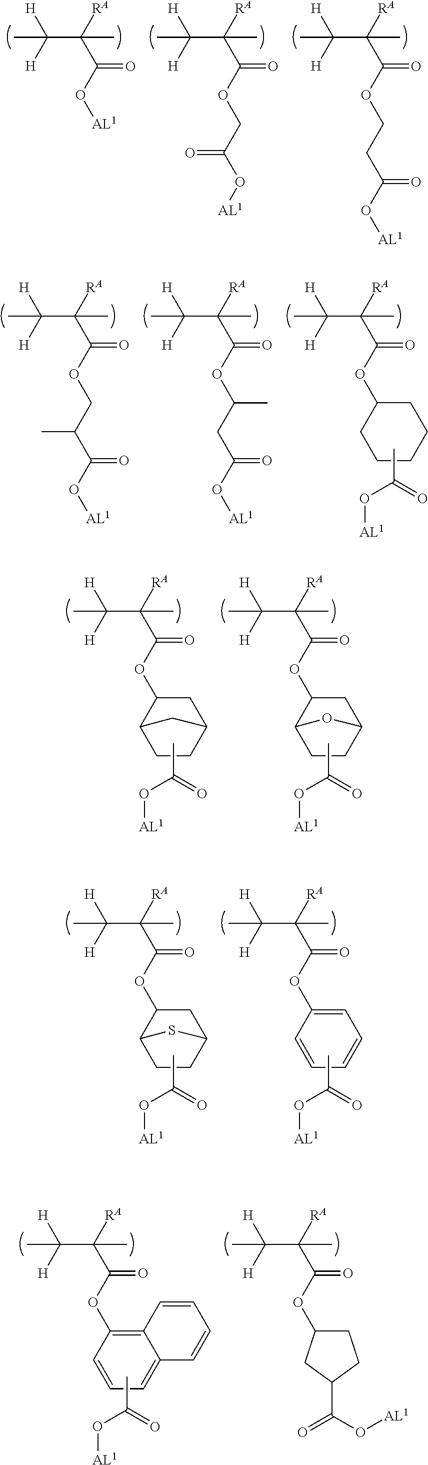

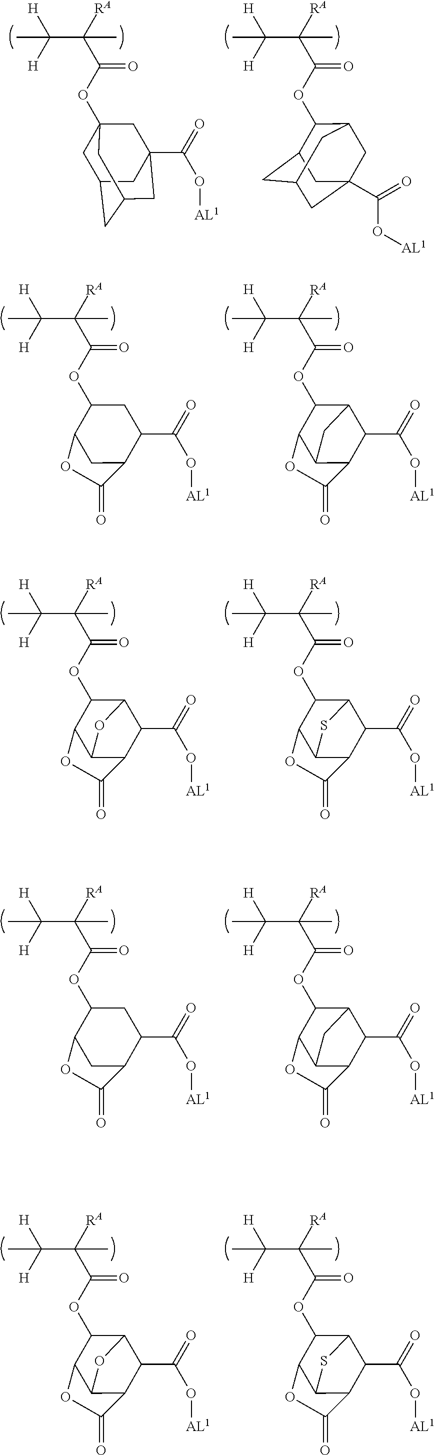

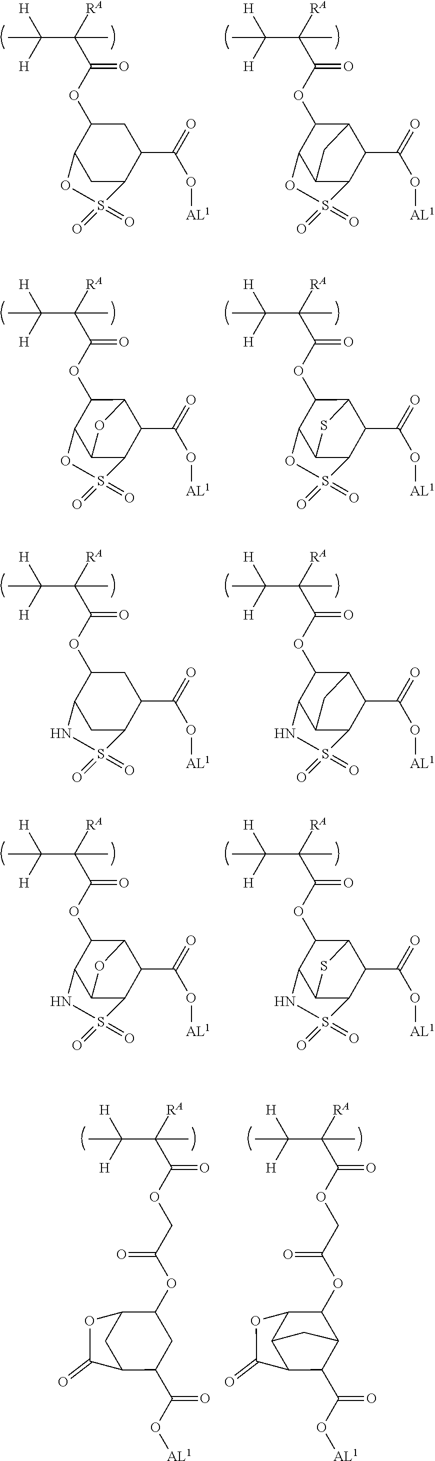

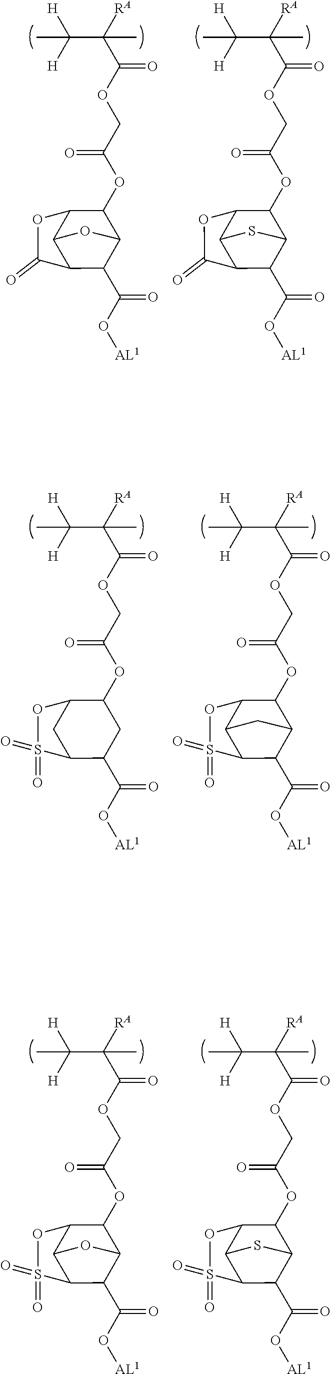

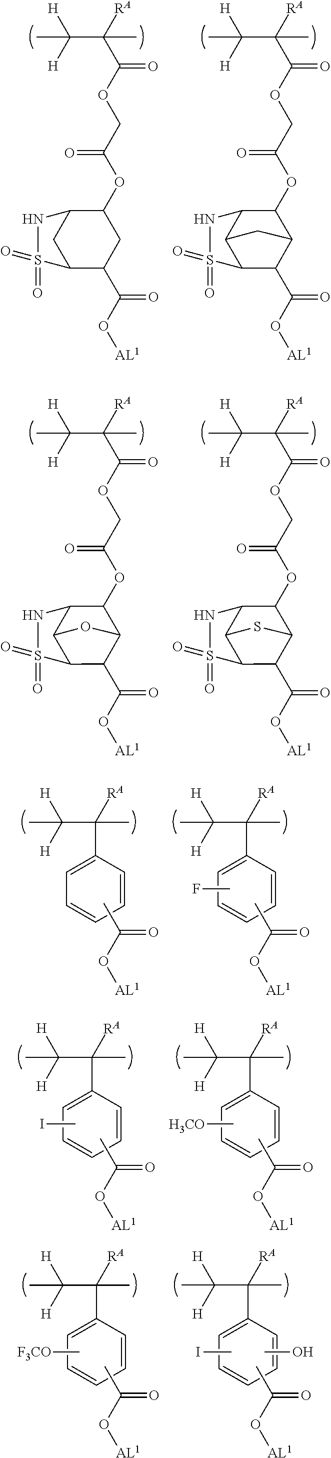

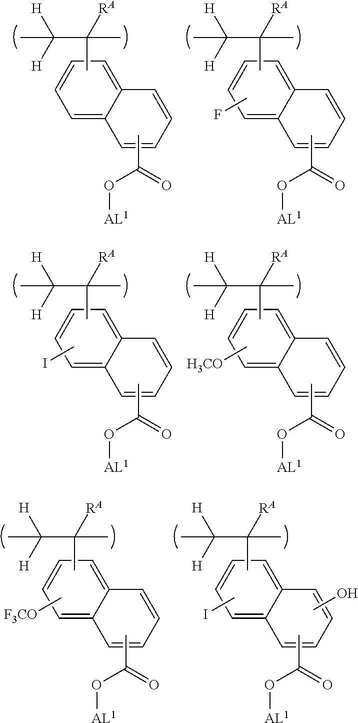

Specific examples of the repeat units a2 are shown below, but are not limited thereto. Herein R^{A} and AL² are as defined above.

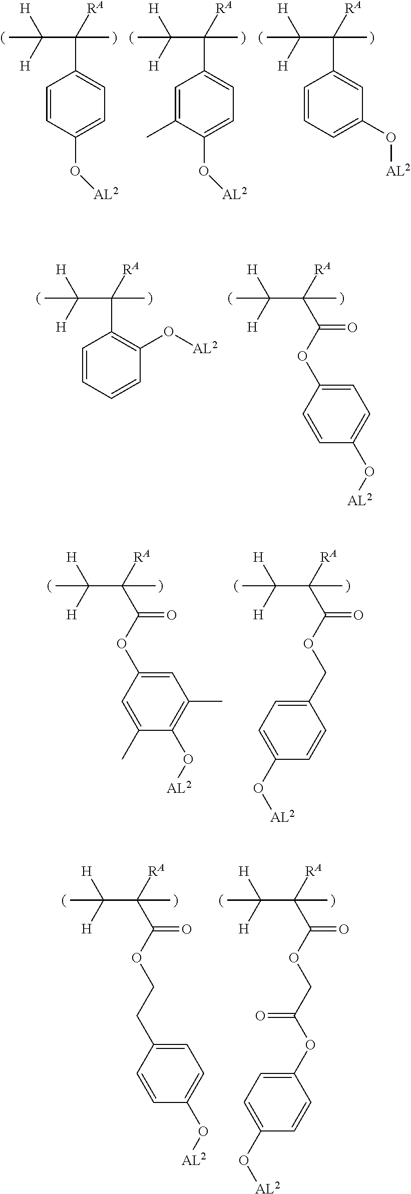

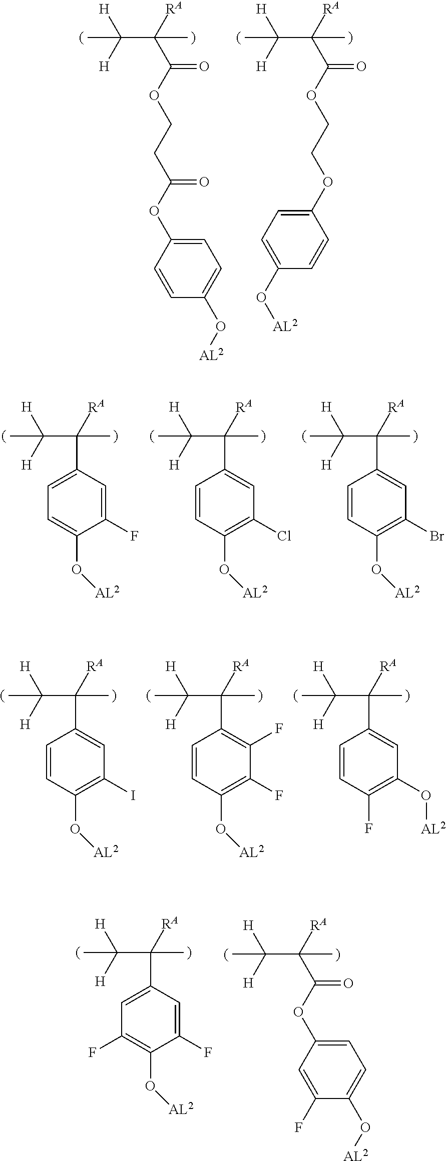

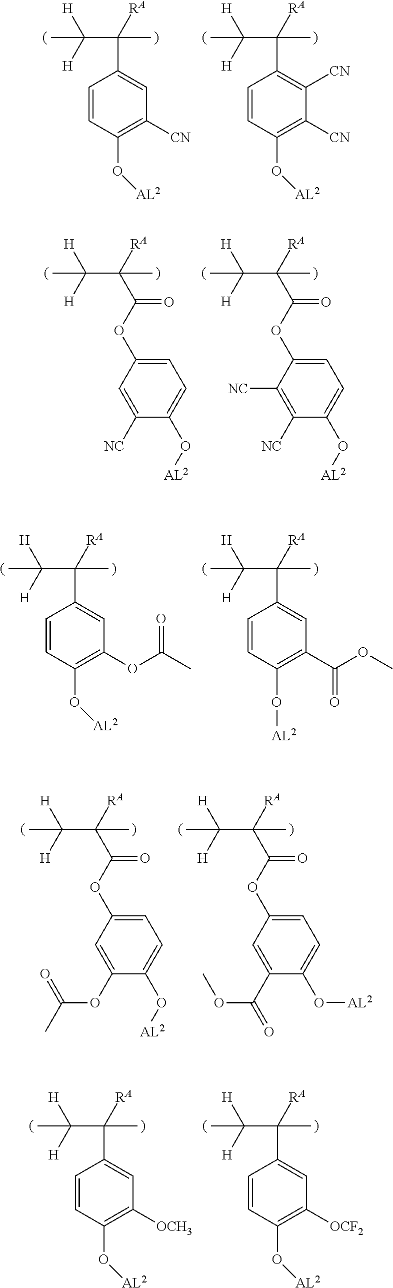

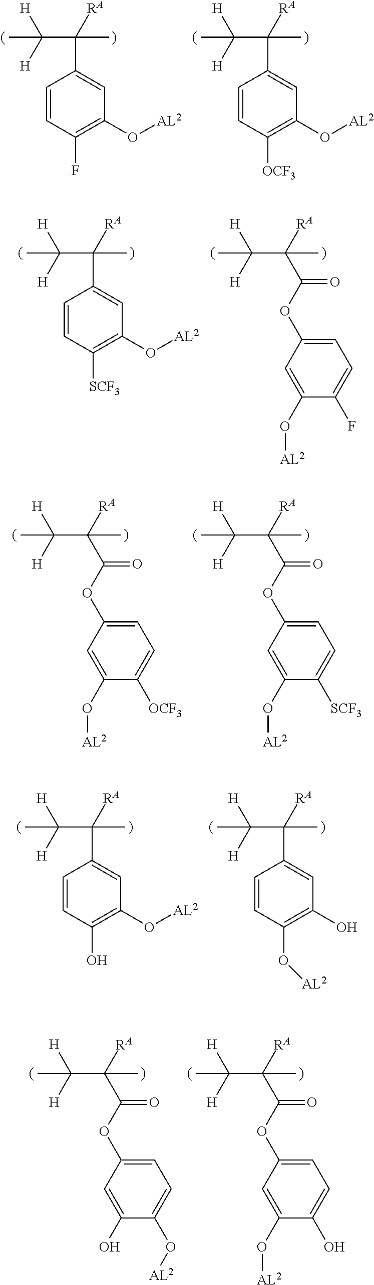

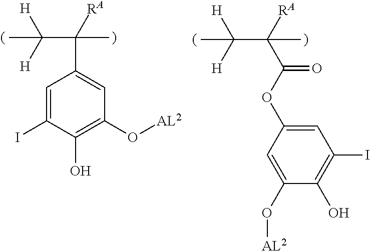

When the chemically amplified resist composition of the present invention is a positive type, the base polymer may comprise repeat units having the formula (a3) (hereinafter, also referred to as repeat units a3).

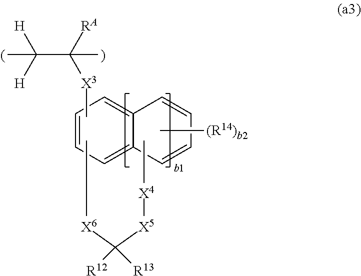

In formula (a3), b1 is 0 or 1. The relevant structure is a benzene ring when b1=0, and a naphthalene ring when b1=1. From the viewpoint of solvent solubility, the benzene ring corresponding to b1=0 is preferred. b2 is 0, 1, 2, or 3 when b1 is 0, and is 0, 1, 2, 3, 4, or 5 when b1 is 1. From the viewpoint of reactant availability, b2 is preferably 0, 1, 2, or 3, and more preferably 0, 1, or 2.

In formula (a3), R^{A} is a hydrogen atom, a fluorine atom, a methyl group, or a trifluoromethyl group. Among them, a hydrogen atom and a methyl group are preferred, and a hydrogen atom is more preferred.

In formula (a3), X³ is a single bond, *—C(═O)—O—, or *—C(═O)—NH—. * designates a point of attachment to the carbon atom in the backbone. Among them, a single bond and *—C(═O)—O— are preferred, and a single bond is more preferred.

In formula (a3), X⁴ is a single bond, a C₁-C₄ aliphatic hydrocarbylene group, a carbonyl group, a sulfonyl group, or a group obtained by combining the foregoing. Among them, a single bond, a carbonyl group, or a sulfonyl group is preferred from the viewpoint of reactant availability, and a single bond or a carbonyl group is more preferred from the viewpoint of a polar group generated after the reaction.

In formula (a3), X⁵ and X⁶ are each independently an oxygen atom or a sulfur atom. Provided that X⁴ and X⁶ are bonded to vicinal carbon atoms on the aromatic ring. X⁵ and X⁶ may be identical or different, but X⁵ and X⁶ are both preferably an oxygen atom from the viewpoint of reactivity.

In formula (a3), R¹² and R¹³ are each independently a hydrogen atom or a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as a methyl group, an ethyl group, an n-propyl group, an isopropyl group, an n-butyl group, an isobutyl group, a sec-butyl group, a tert-butyl group, an n-pentyl group, an n-hexyl group, an n-octyl group, an n-nonyl group, an n-decyl group, an undecyl group, a dodecyl group, a tridecyl group, a tetradecyl group, a pentadecyl group, a heptadecyl group, an octadecyl group, a nonadecyl group, and an icosyl group; C₃-C₂₀ cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group and a naphthyl group; C₇-C₂₀ aralkyl groups such as a benzyl group, a 1-phenylethyl group, and a 2-phenylethyl group; and combinations thereof. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

R¹² and R¹³ may bond together to form a ring with the carbon atom to which they are attached. Specific examples of the ring thus formed include a cyclopropane ring, a cyclobutane ring, a cyclopentane ring, a cyclohexane ring, a norbornane ring, and an adamantane ring. In the ring, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

In formula (a3), R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}). R^{14A} and R^{14B} are each independently a hydrogen atom or a C₁-C₆ hydrocarbyl group. The halogen atom is preferably a fluorine atom, a chlorine atom, a bromine atom, or an iodine atom, and more preferably a fluorine atom or an iodine atom. The hydrocarbyl group and the hydrocarbyl moiety in the hydrocarbyloxy group, the hydrocarbyloxycarbonyl group, and the hydrocarbylthio group may be saturated or unsaturated and straight, branched, or cyclic. Specific examples thereof are as exemplified above for the hydrocarbyl groups R¹² and R¹³. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. A plurality of R¹⁴ may be identical or different when b2 is 2 or more.

The plurality of R¹⁴ may bond together to form a ring with the carbon atoms of the aromatic ring to which they are attached when b2 is 2 or more. Specific examples of the ring thus formed include a cyclopropane ring, a cyclobutane ring, a cyclopentane ring, a cyclohexane ring, a norbornane ring, and an adamantane ring. In the ring, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Specific examples of the repeat units a3 are shown below, but are not limited thereto. Herein R^{A} is as defined above, and Me is a methyl group. The bond positions of various substituents on the aromatic ring are interchangeable.

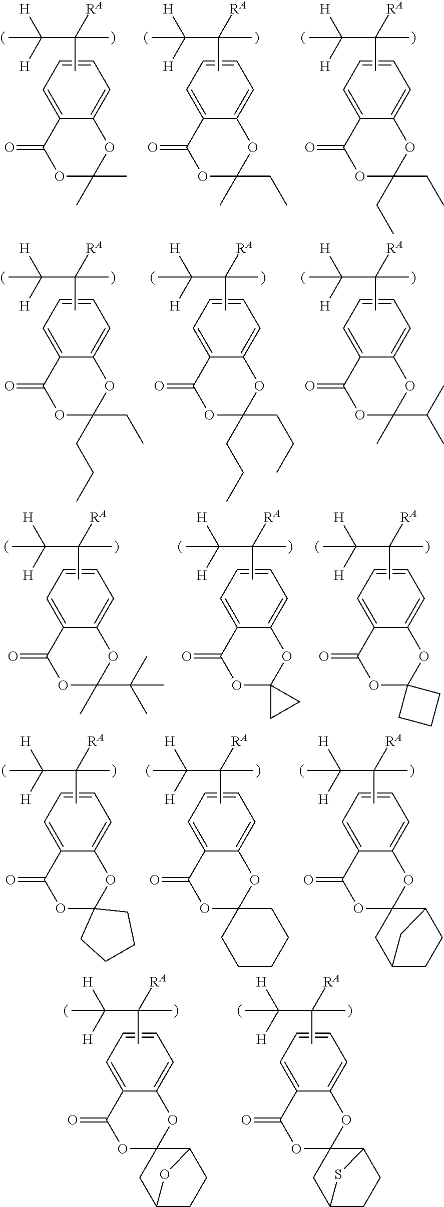

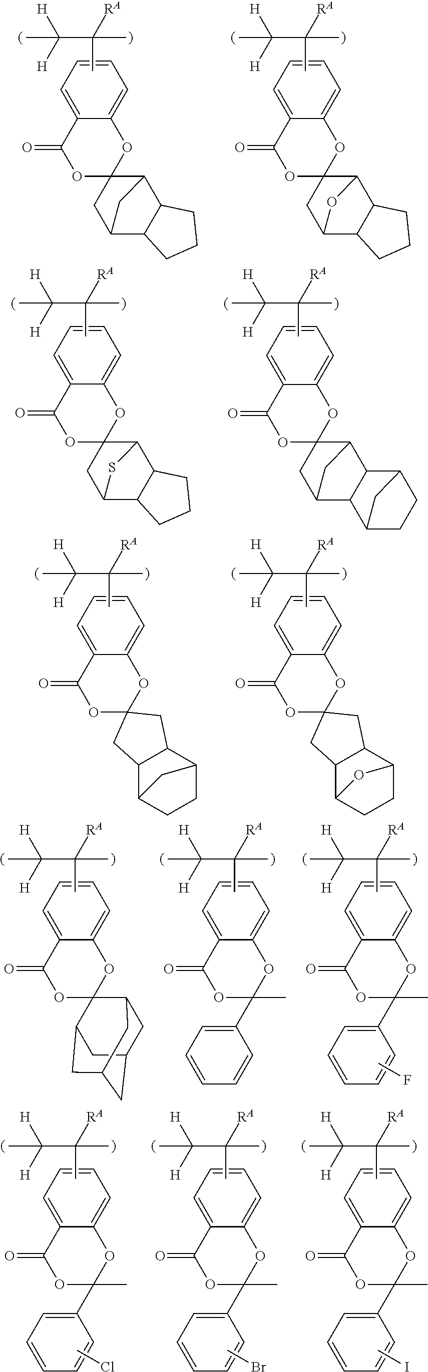

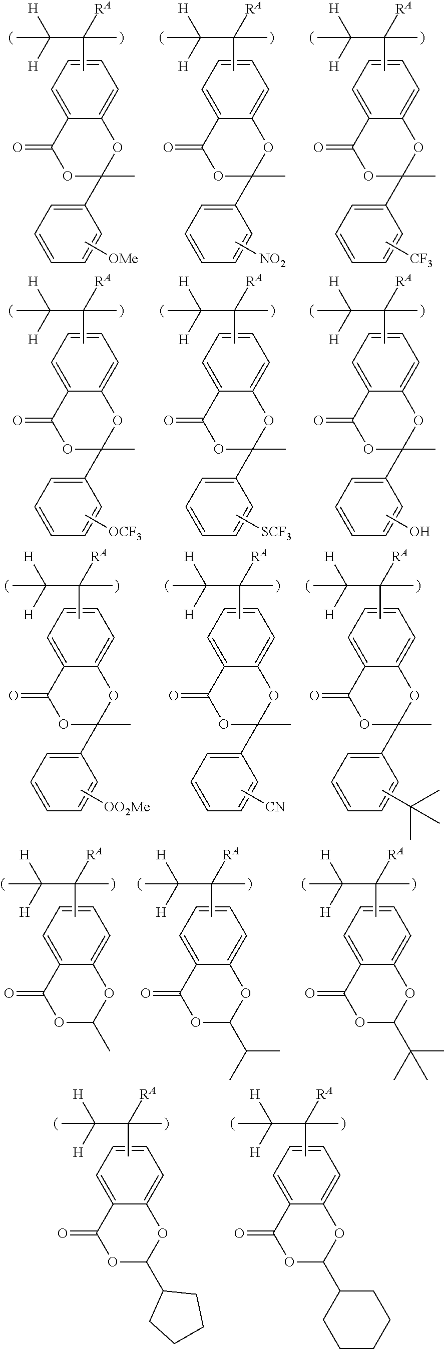

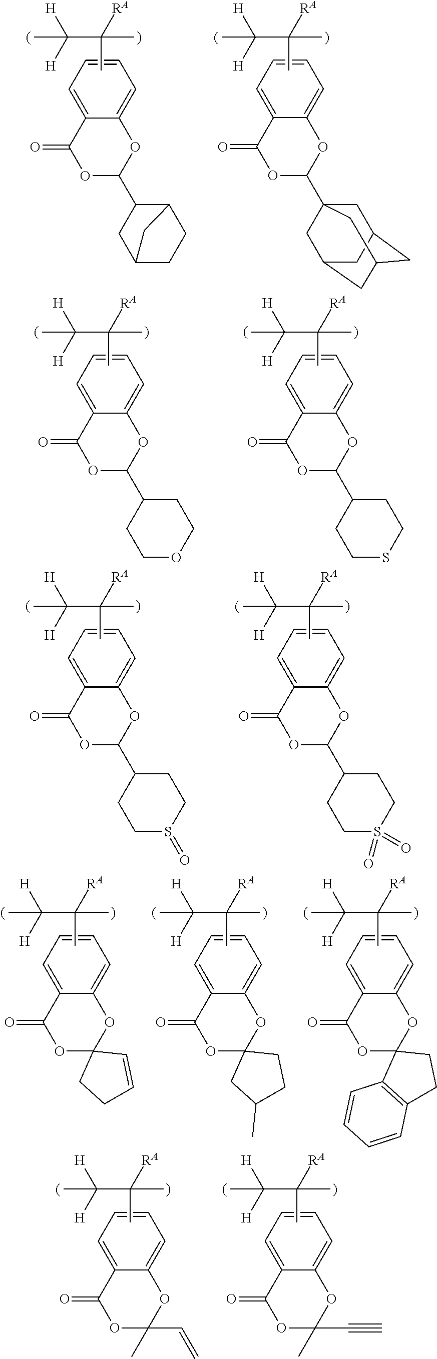

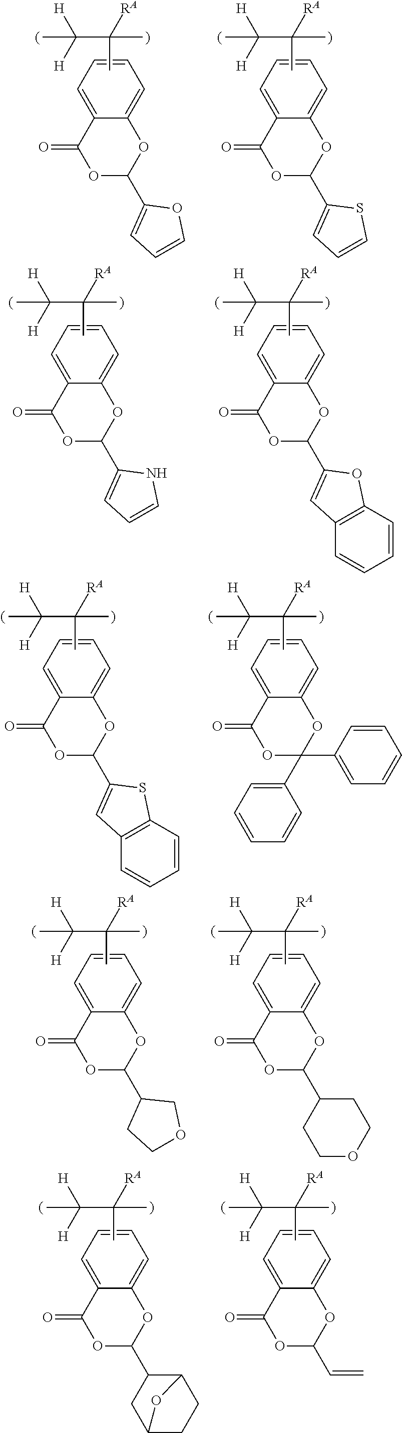

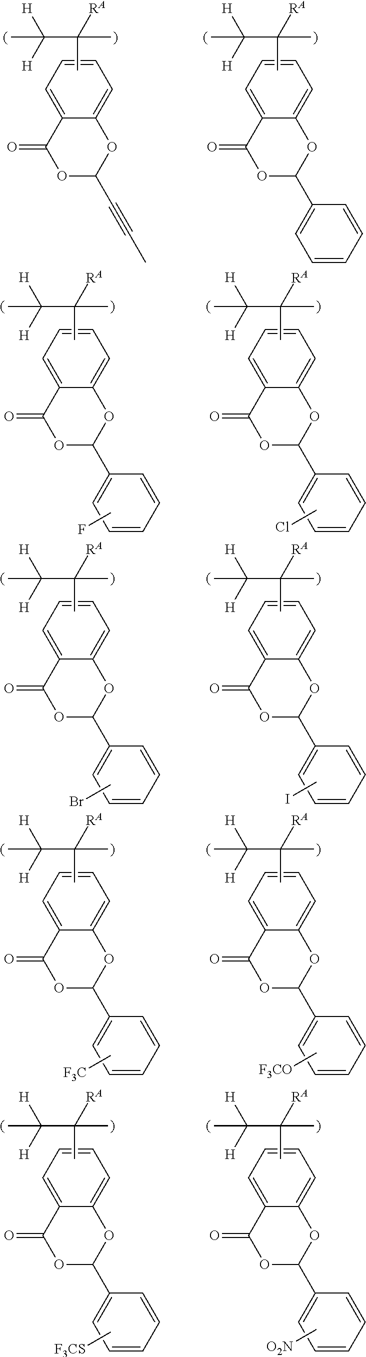

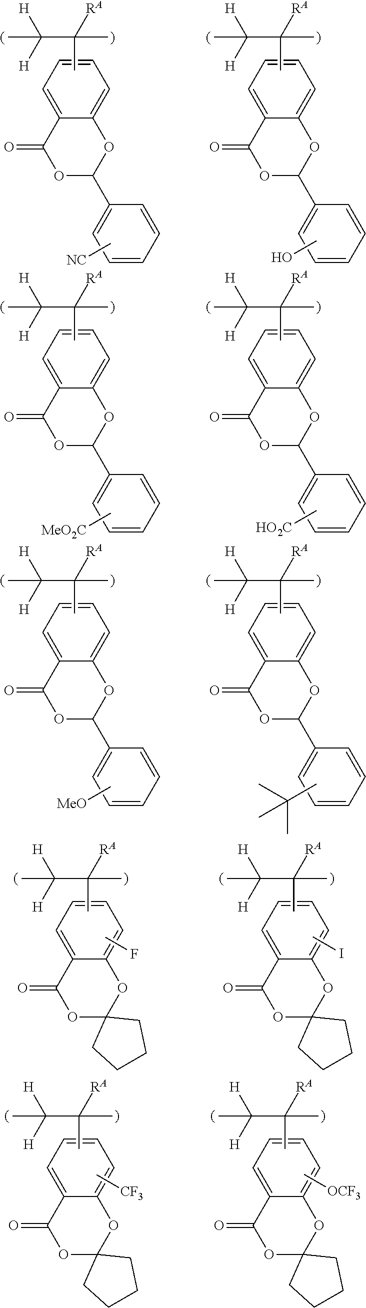

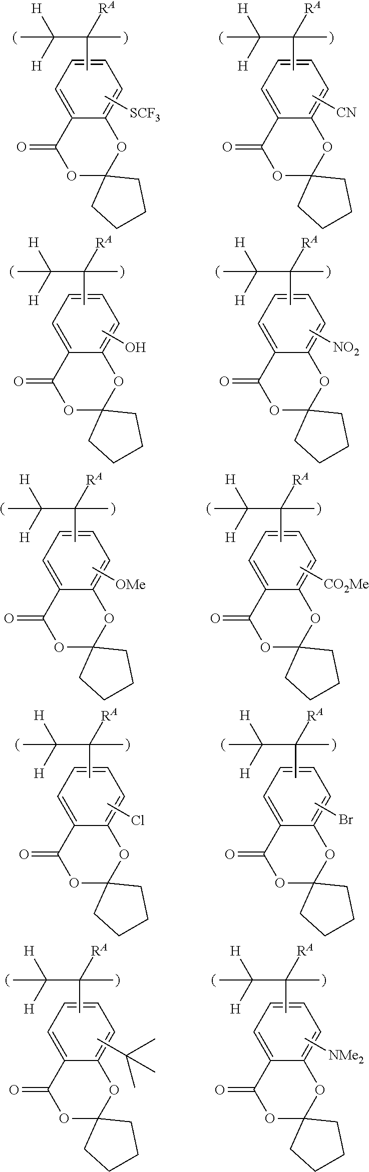

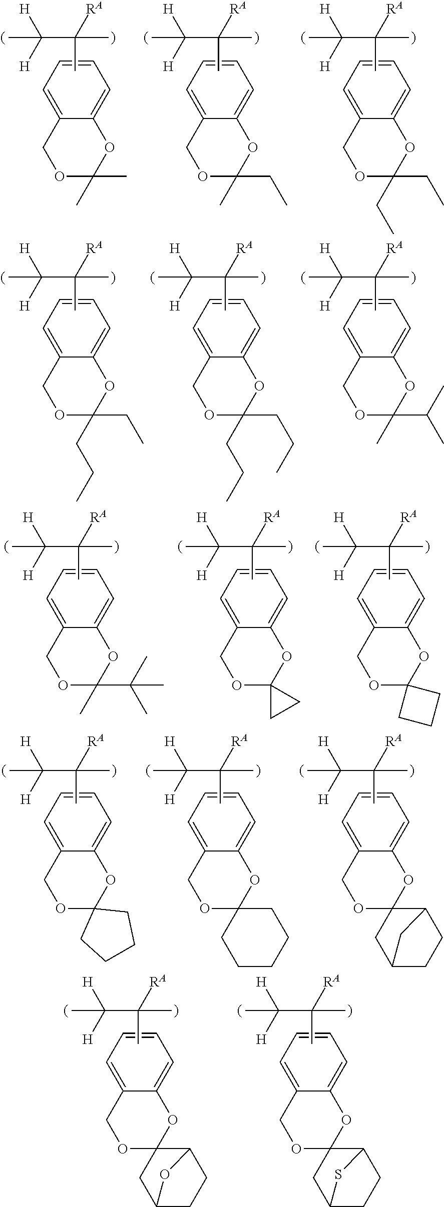

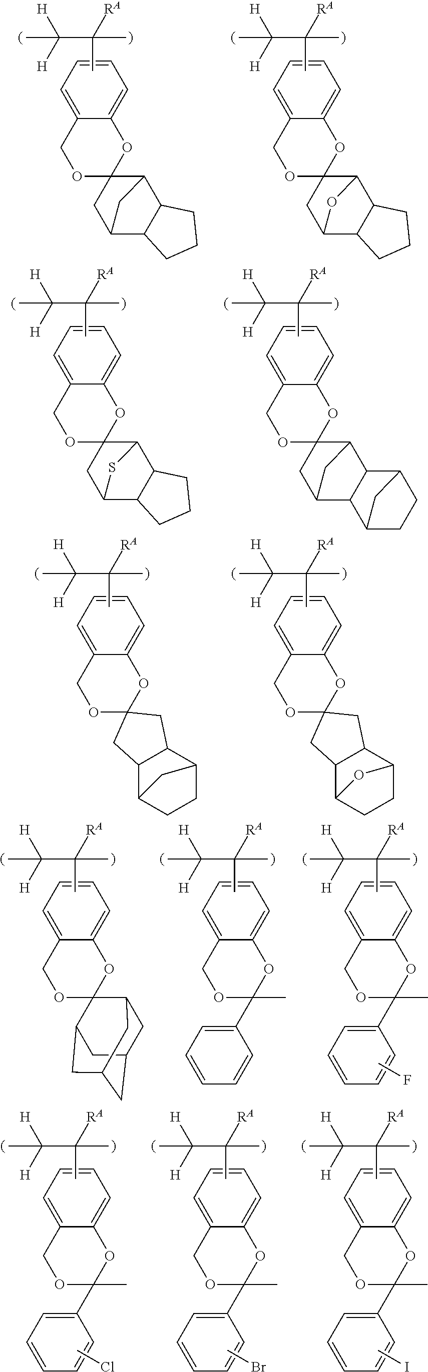

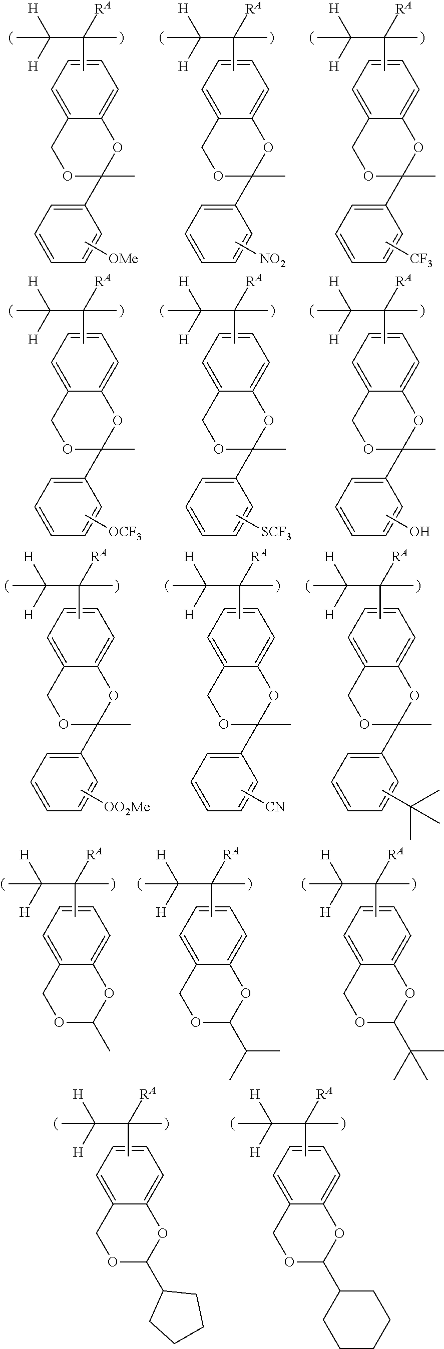

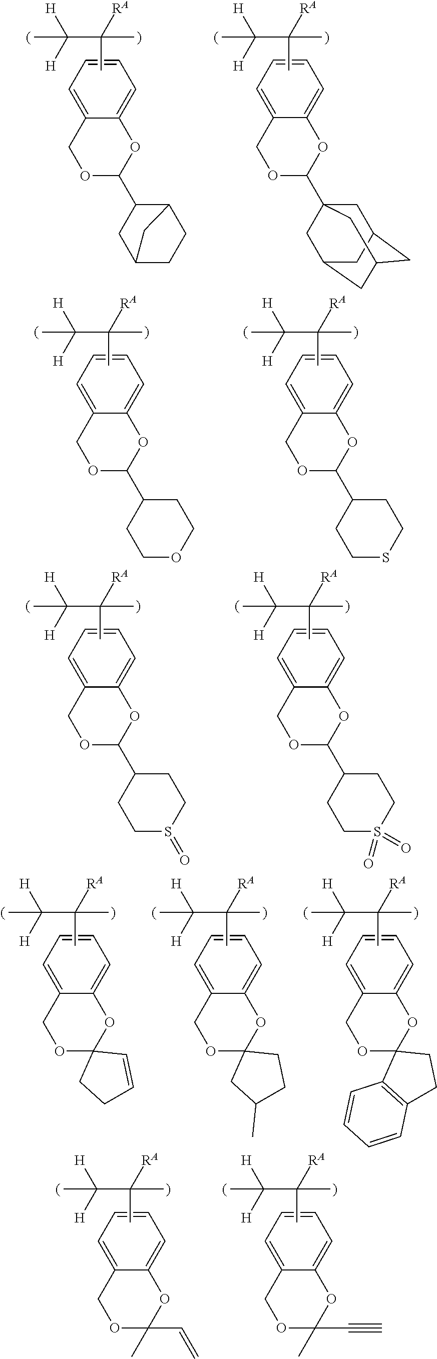

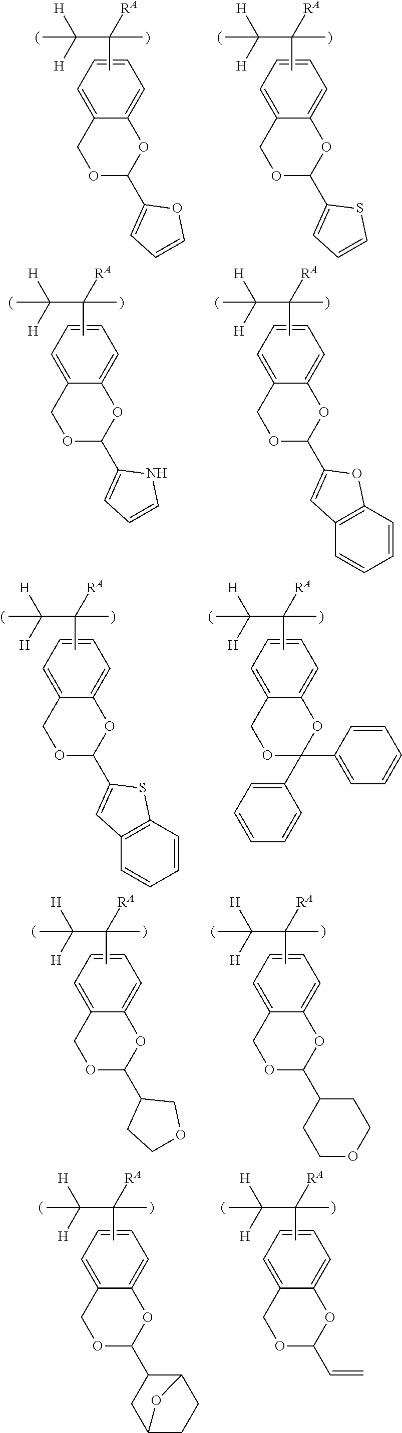

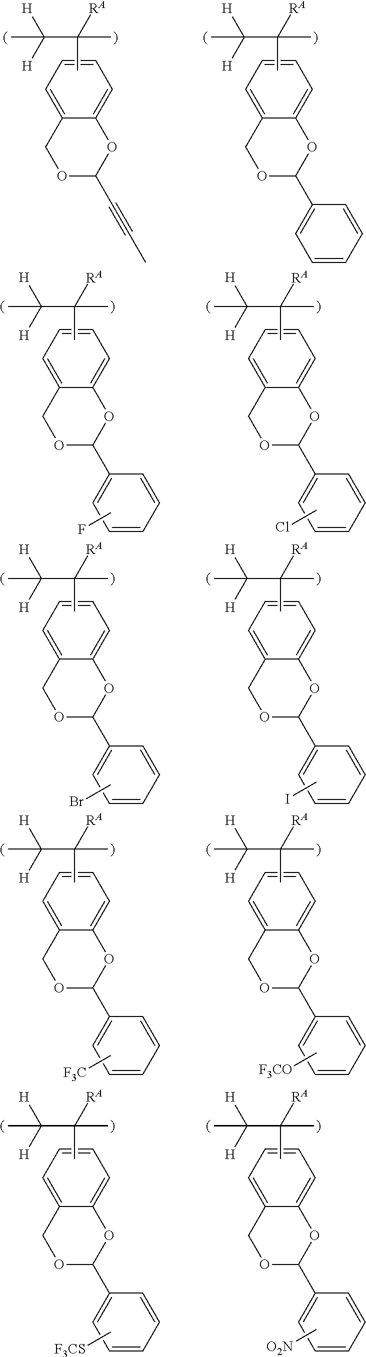

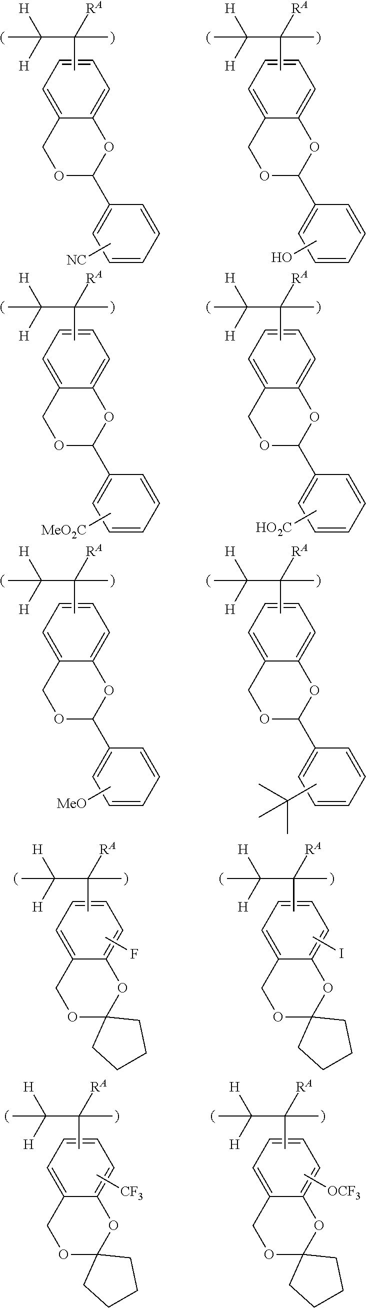

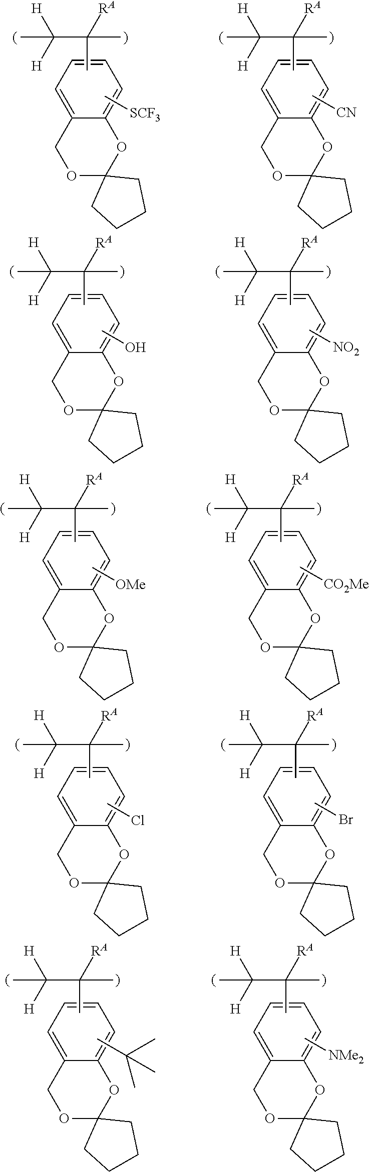

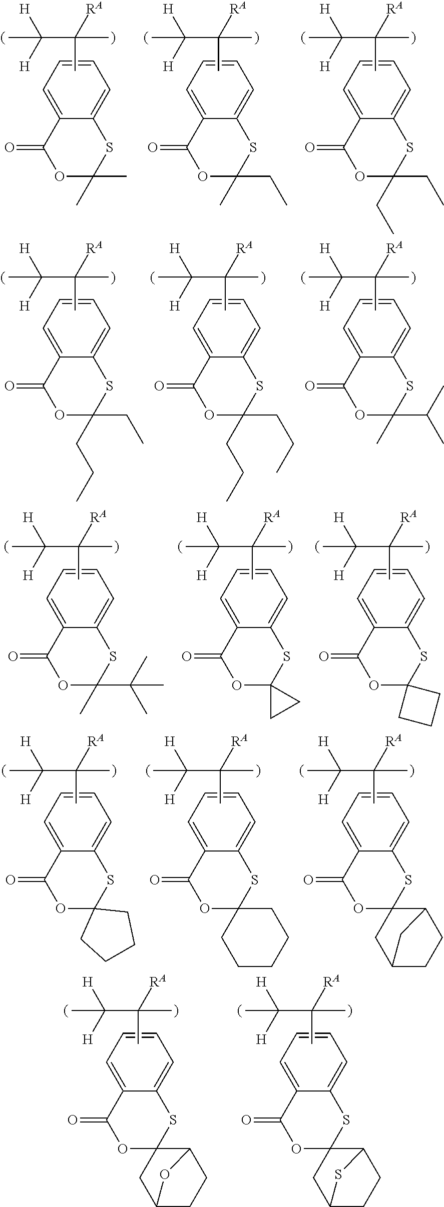

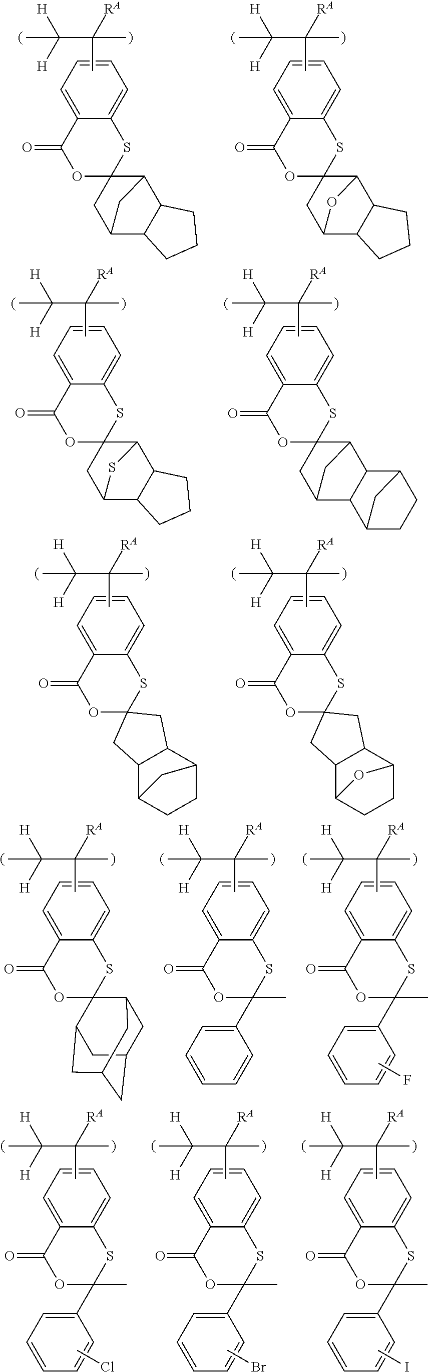

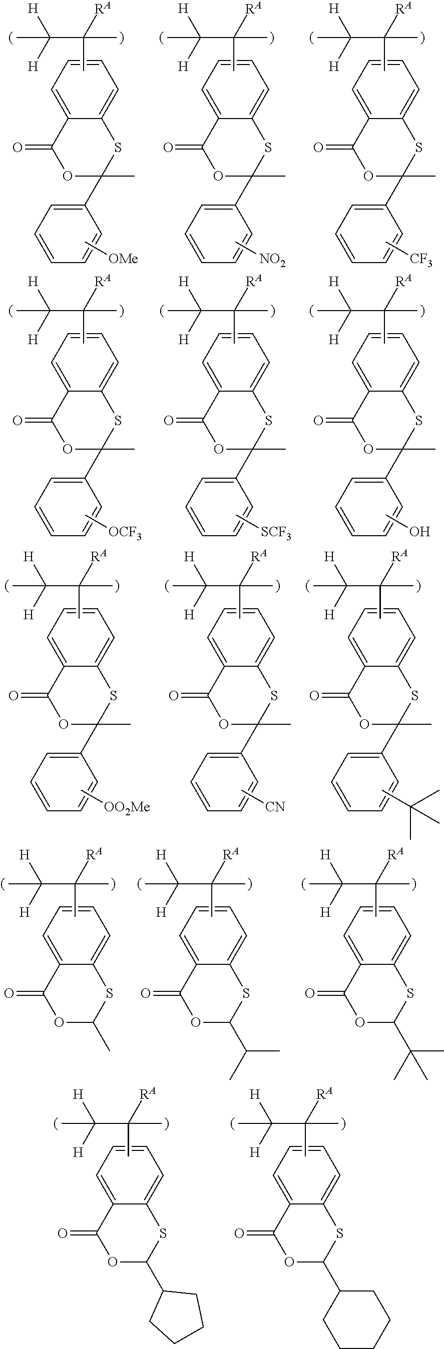

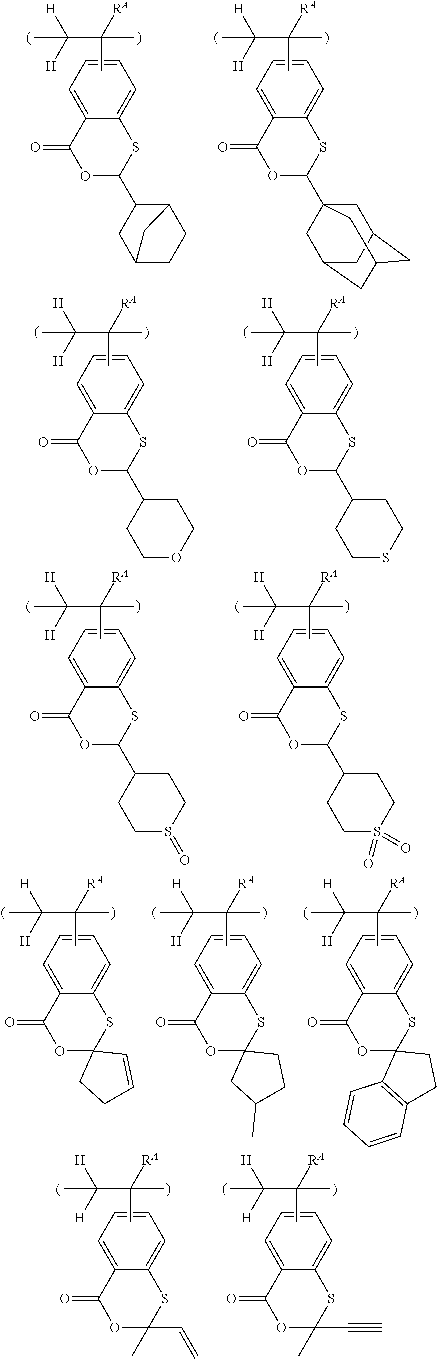

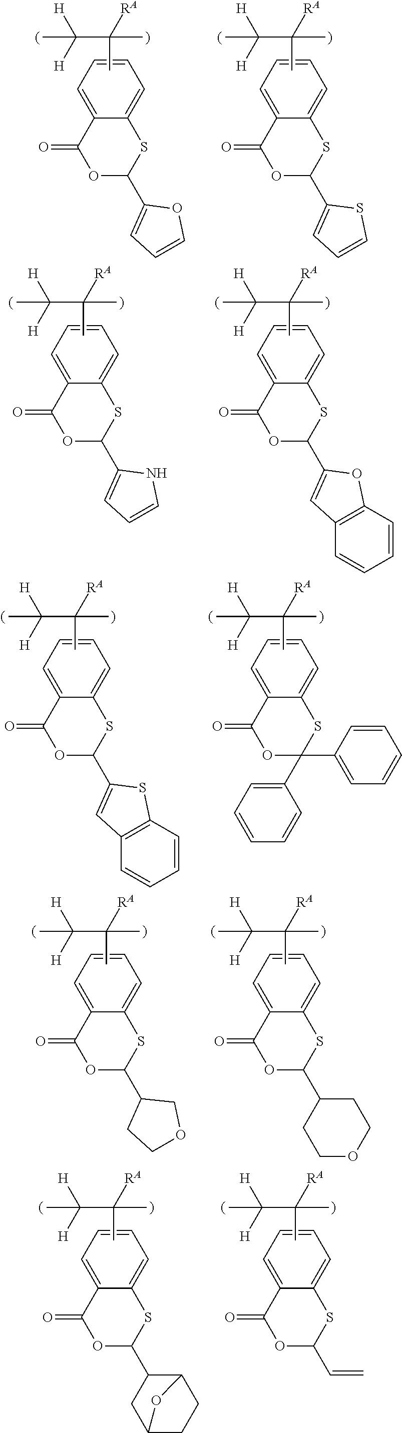

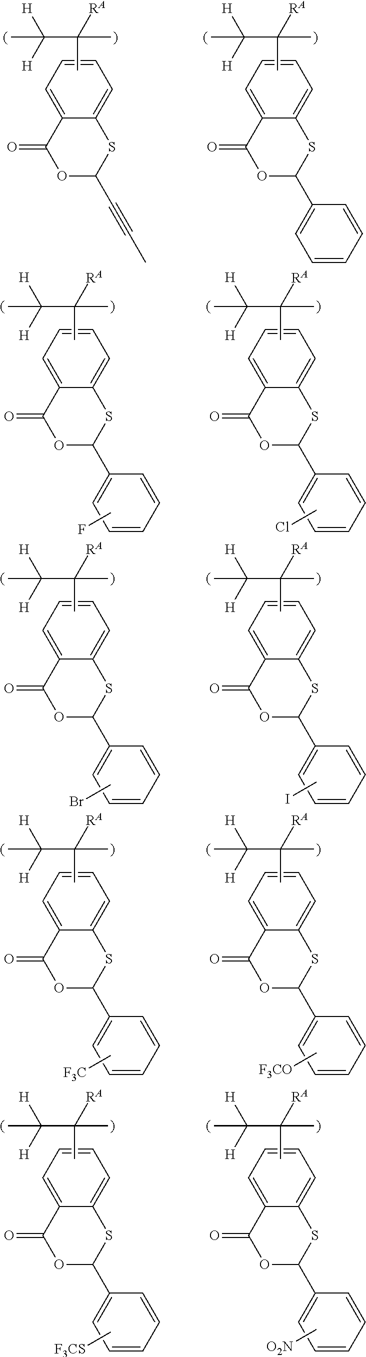

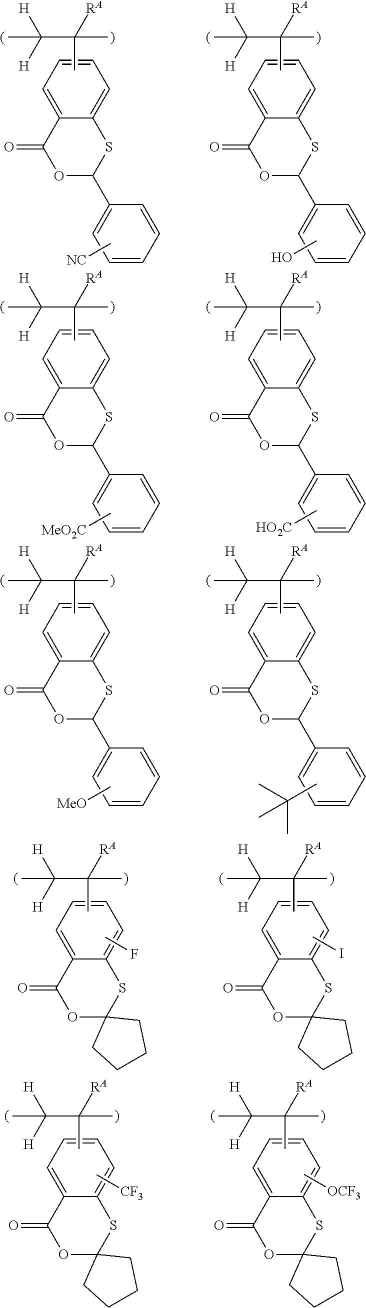

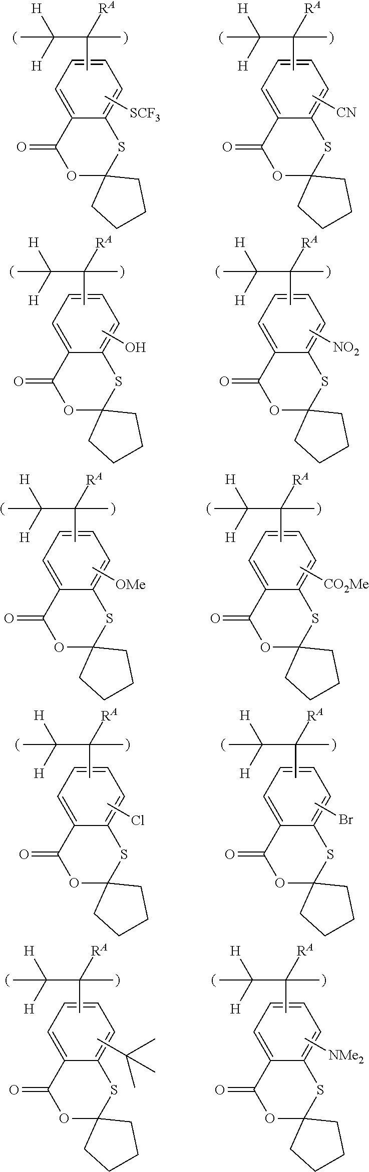

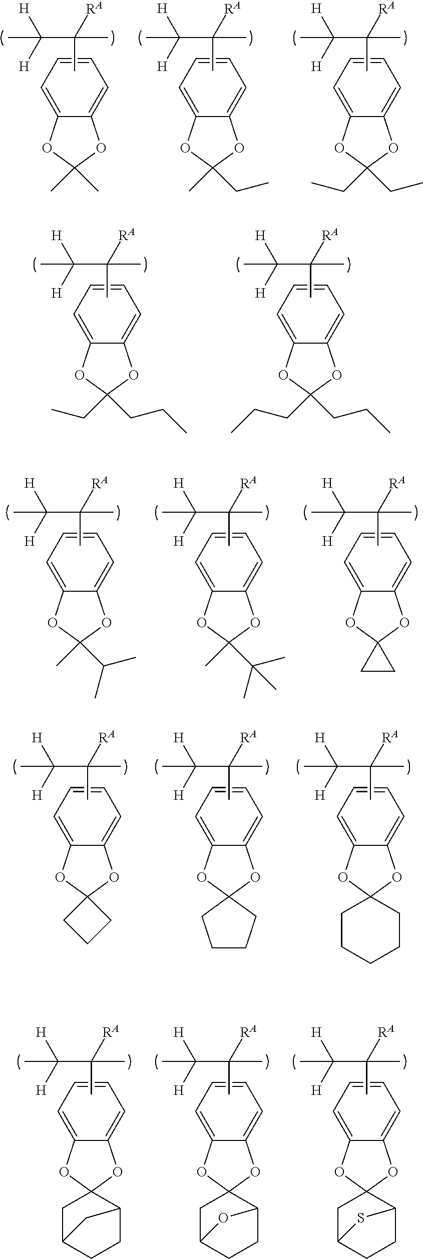

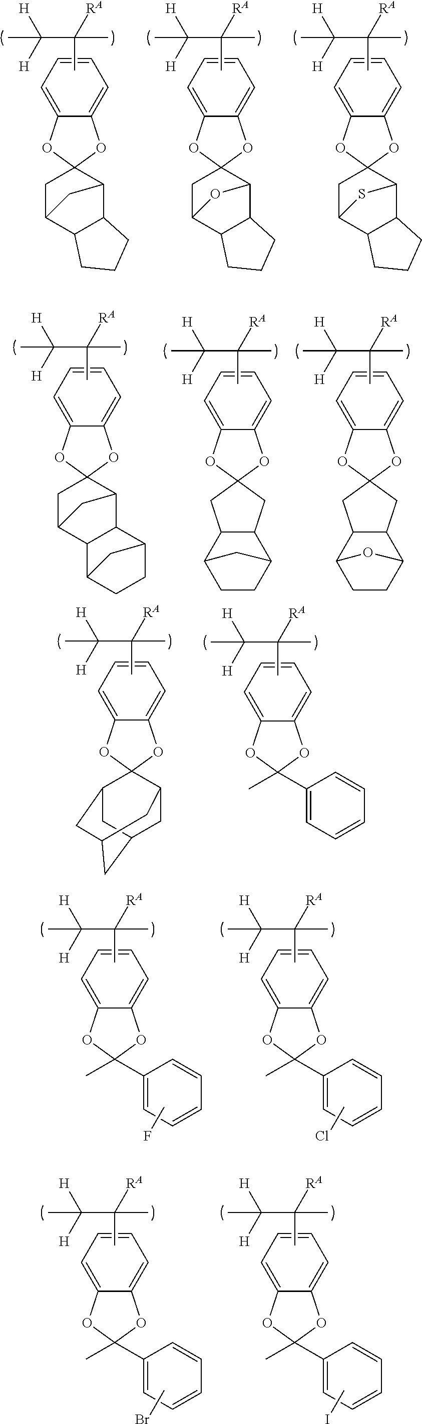

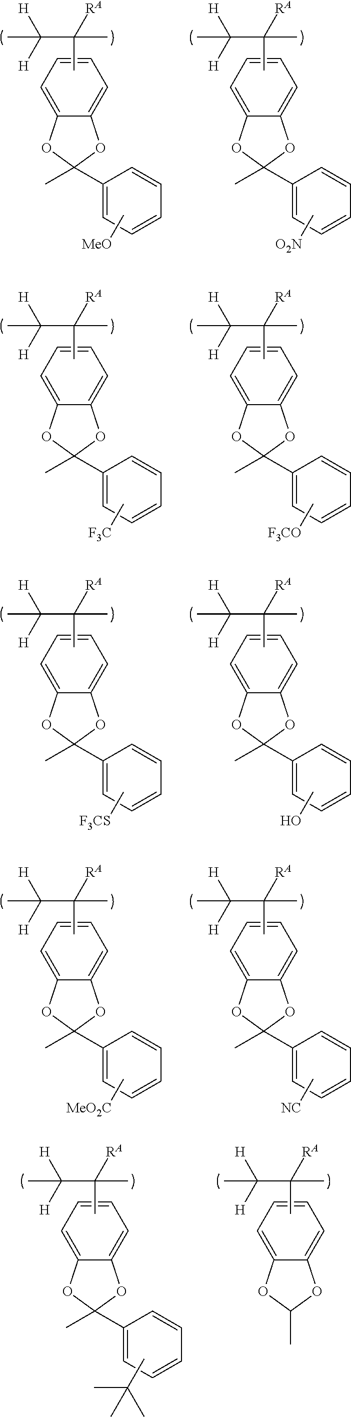

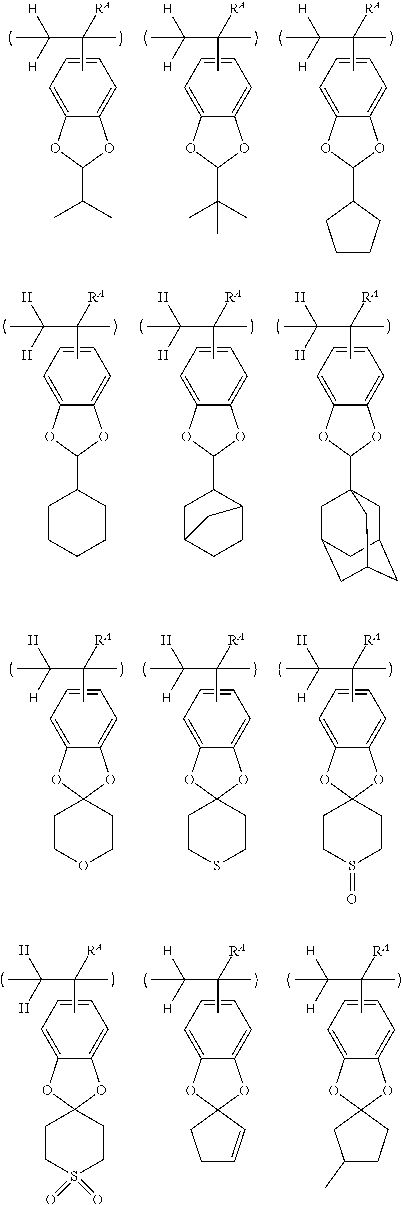

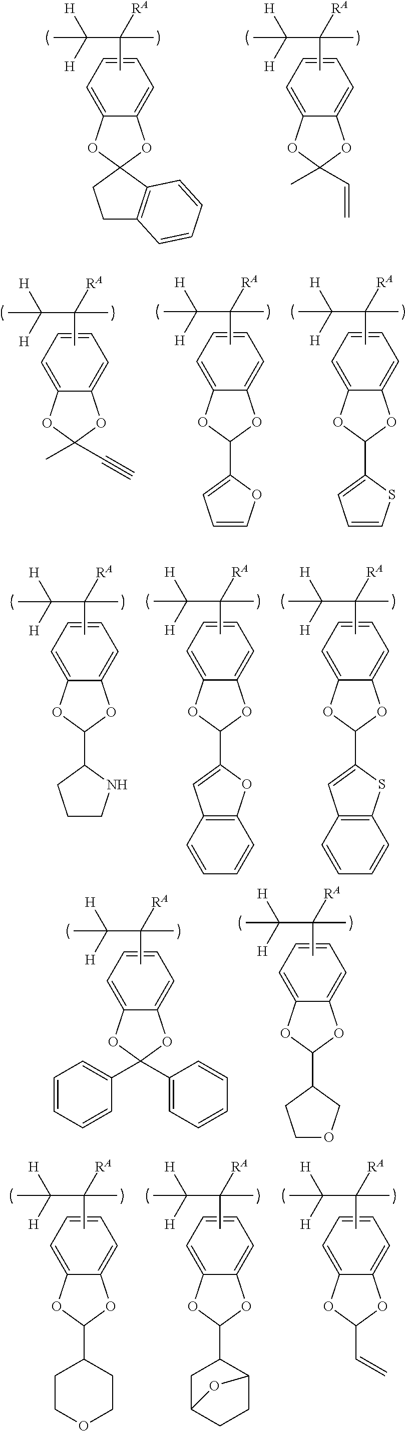

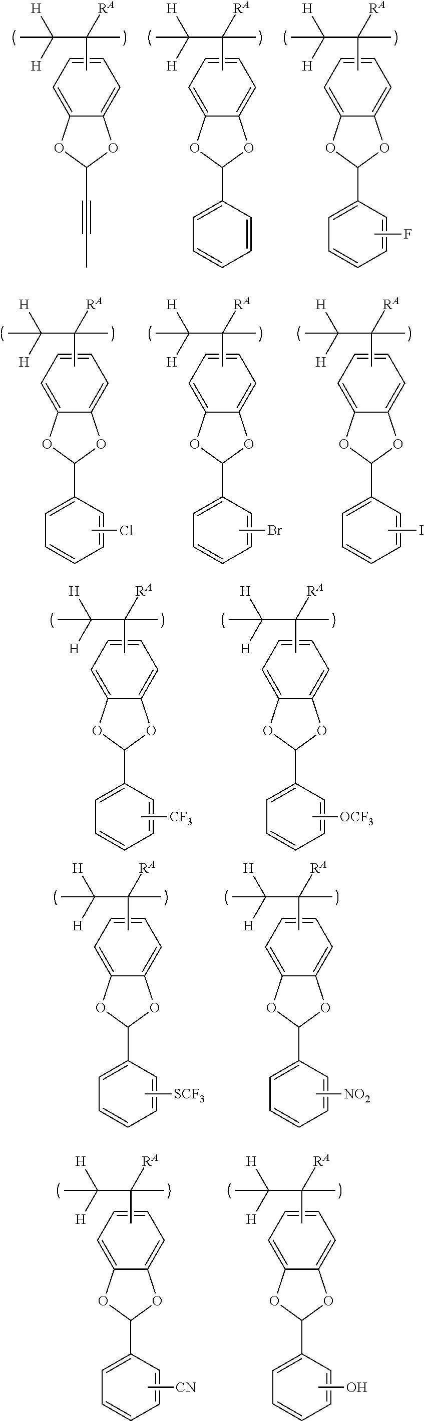

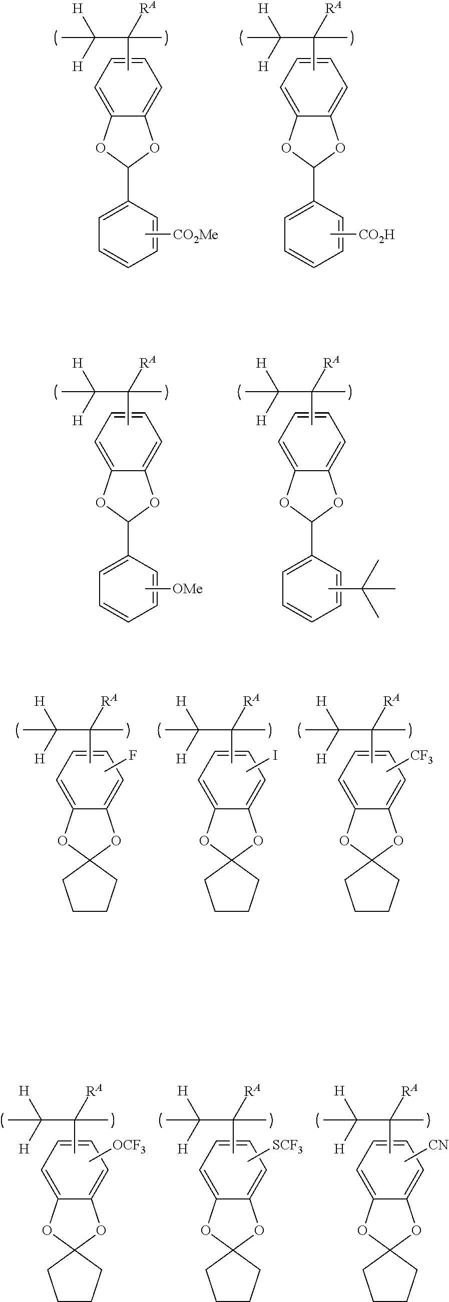

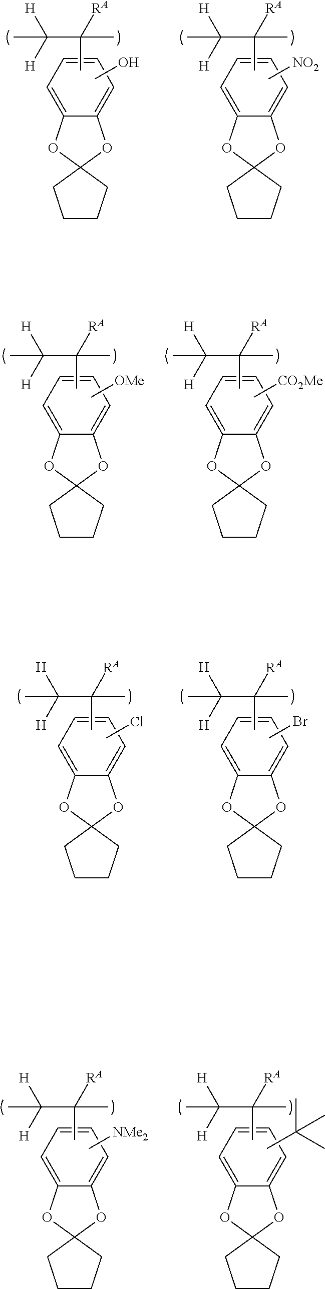

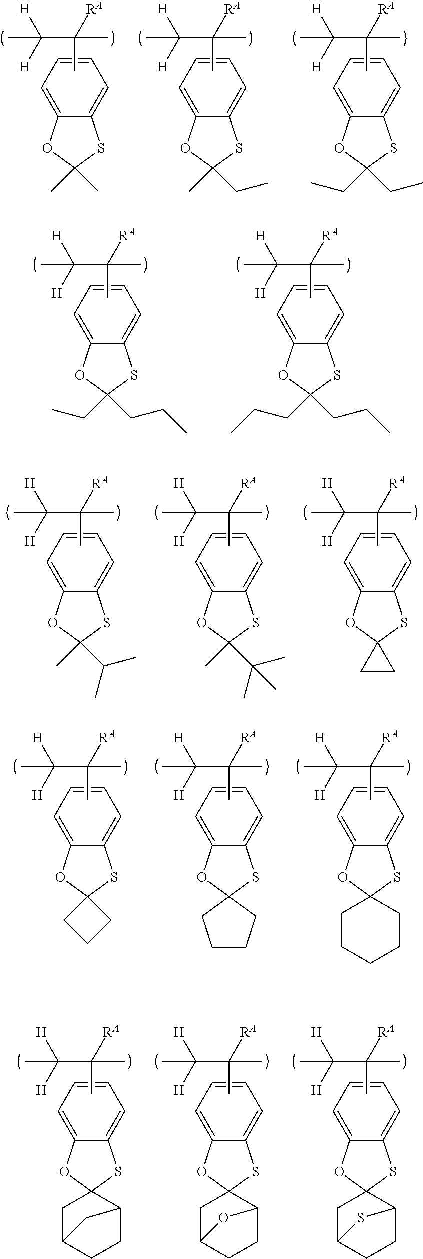

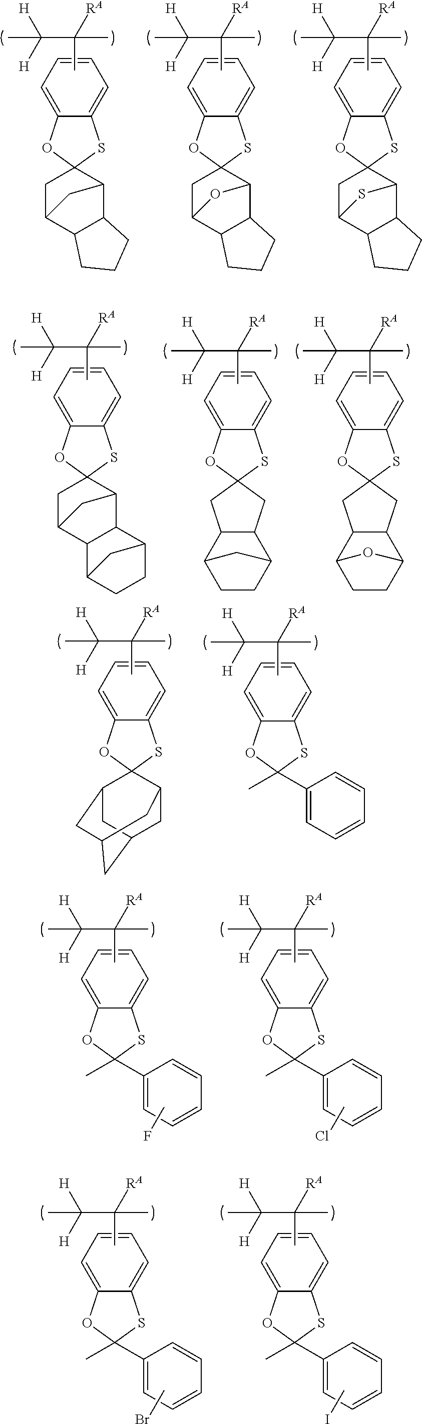

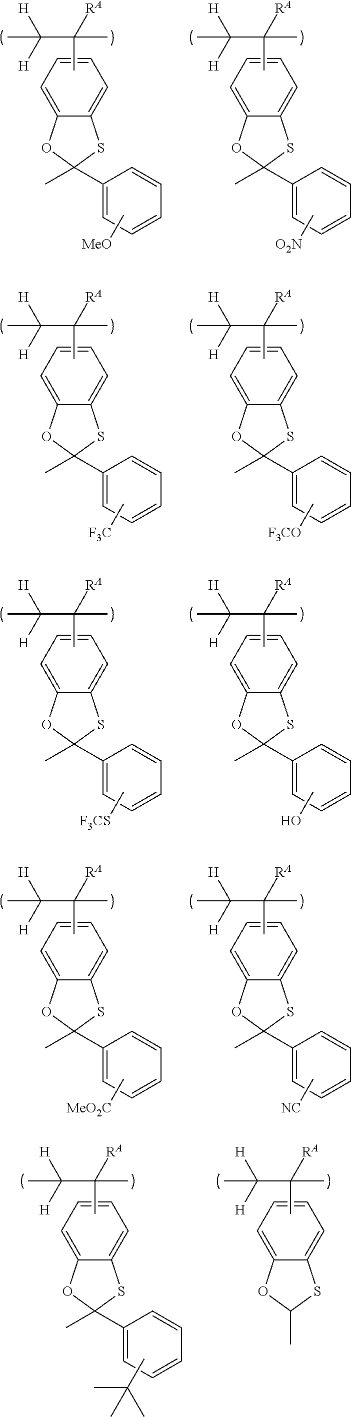

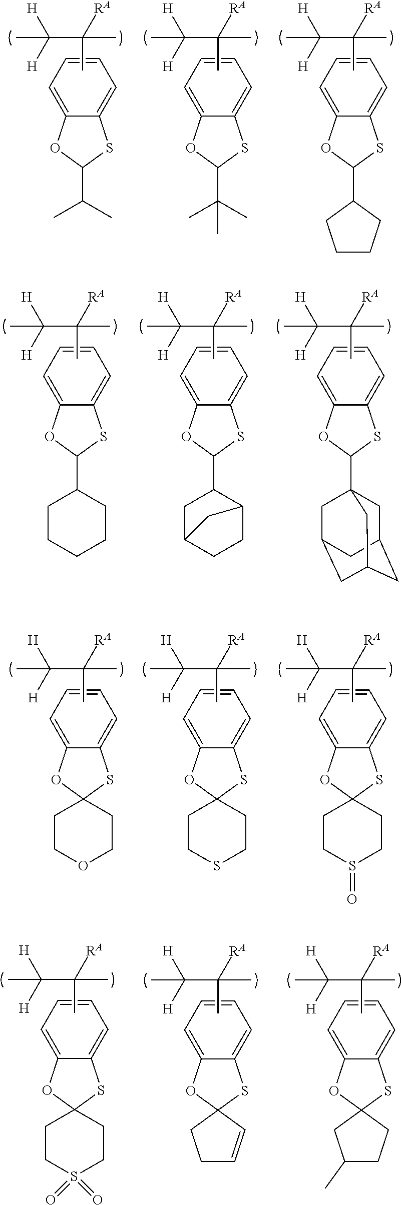

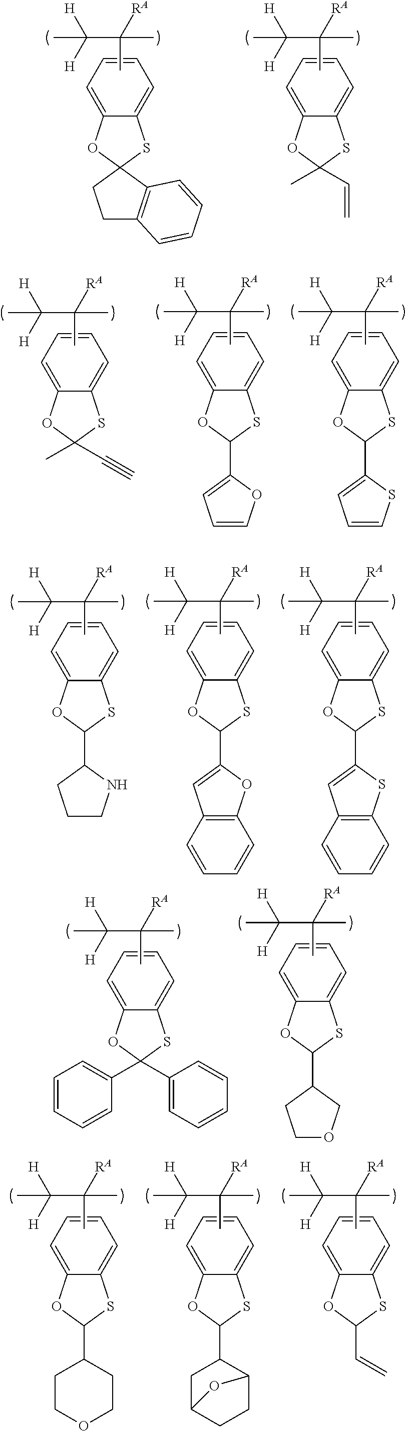

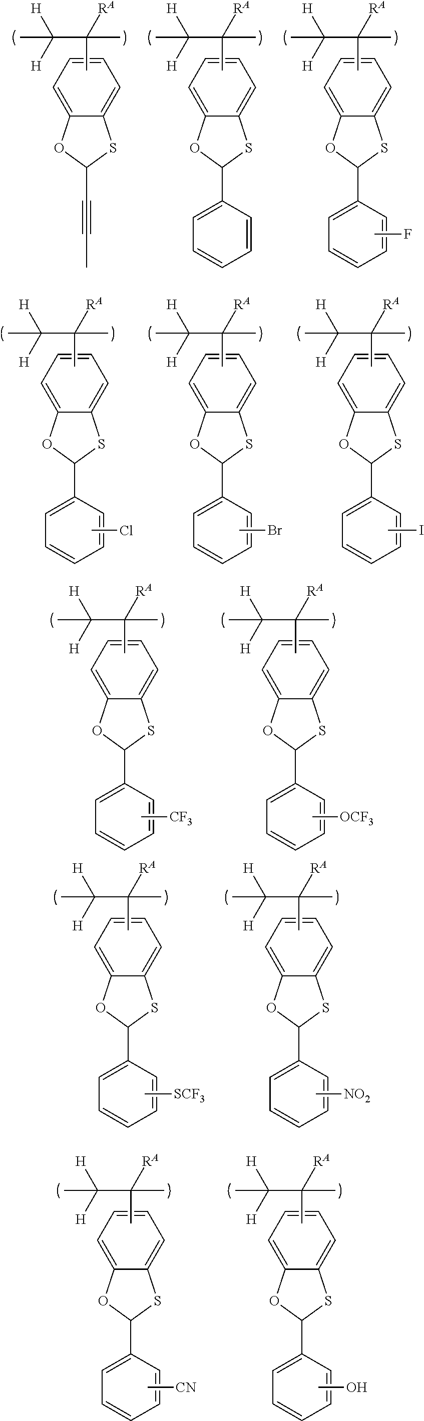

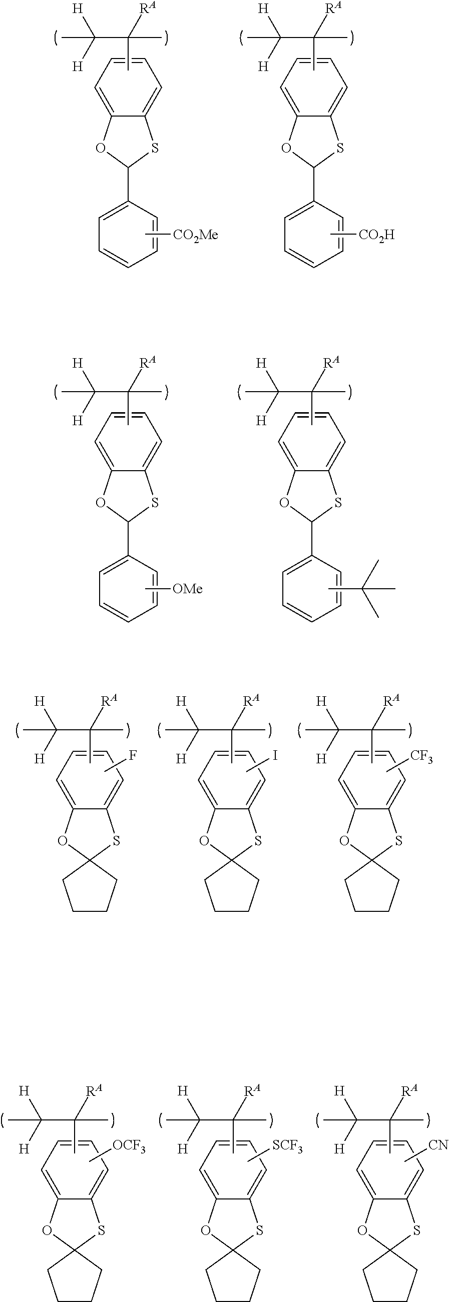

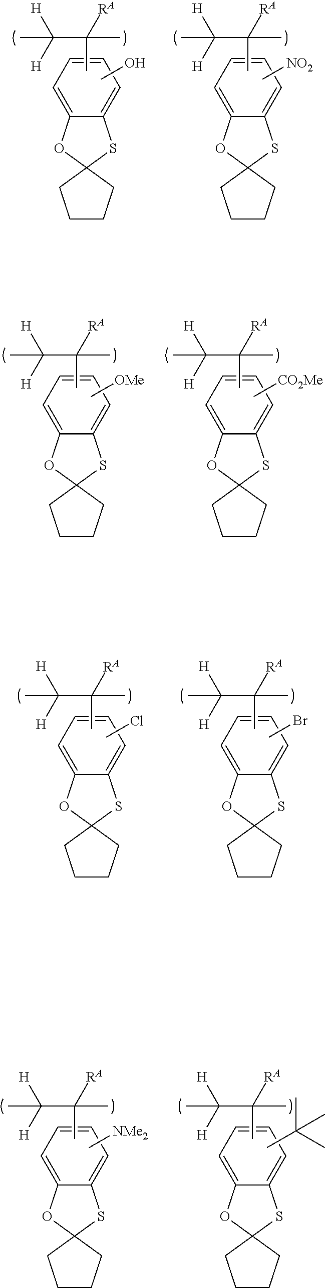

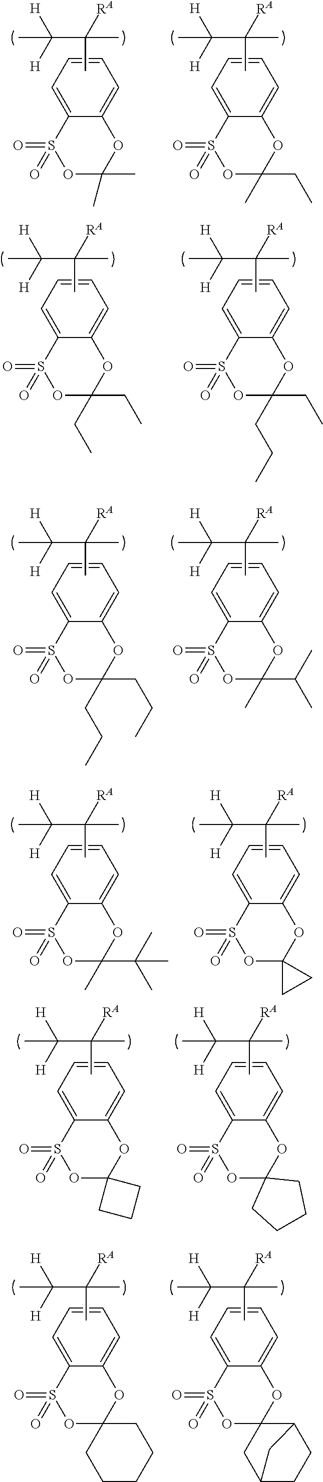

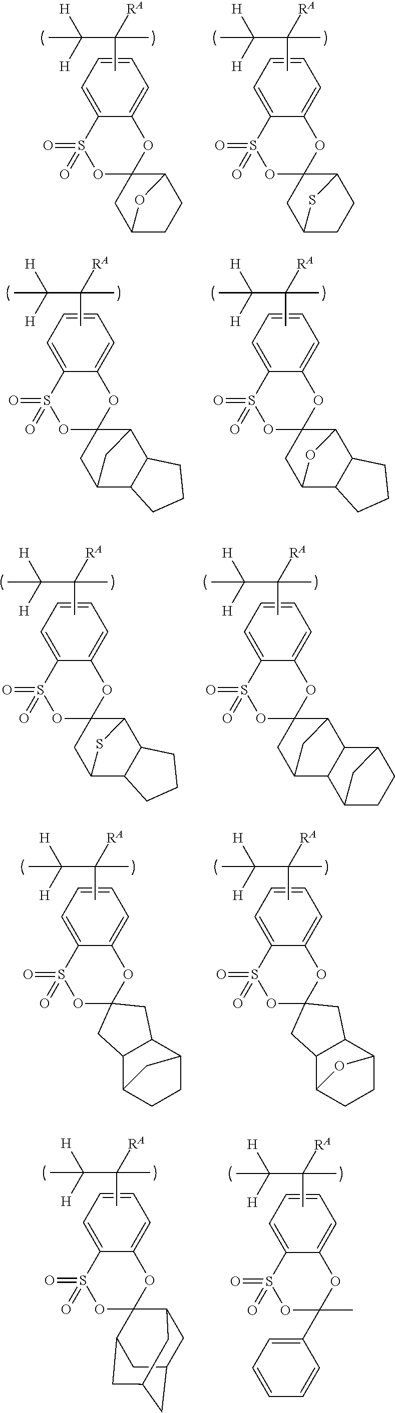

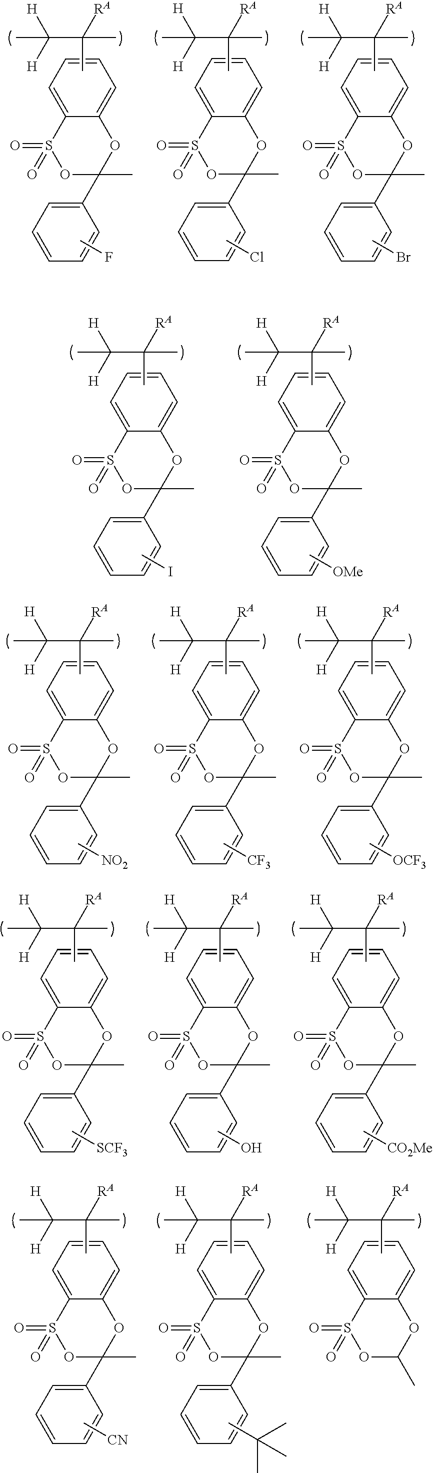

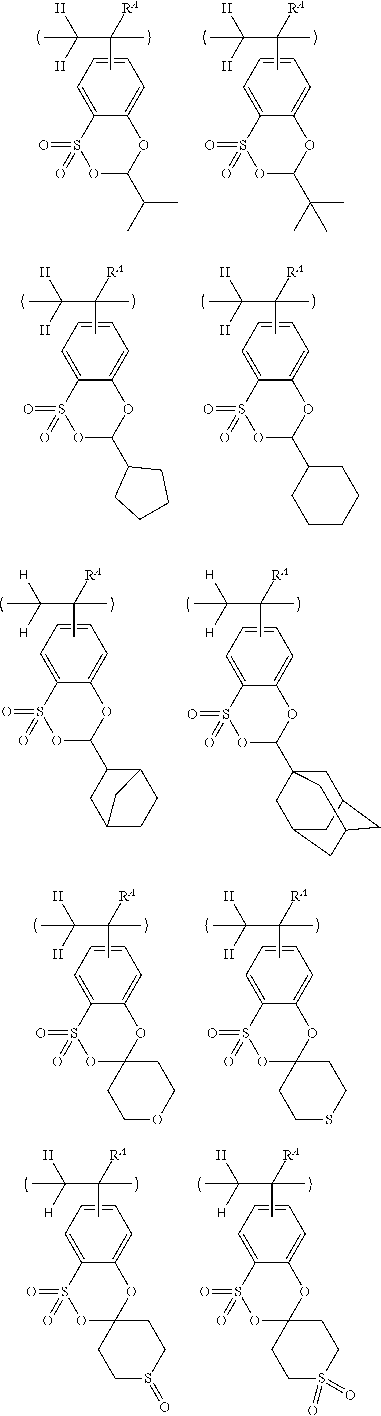

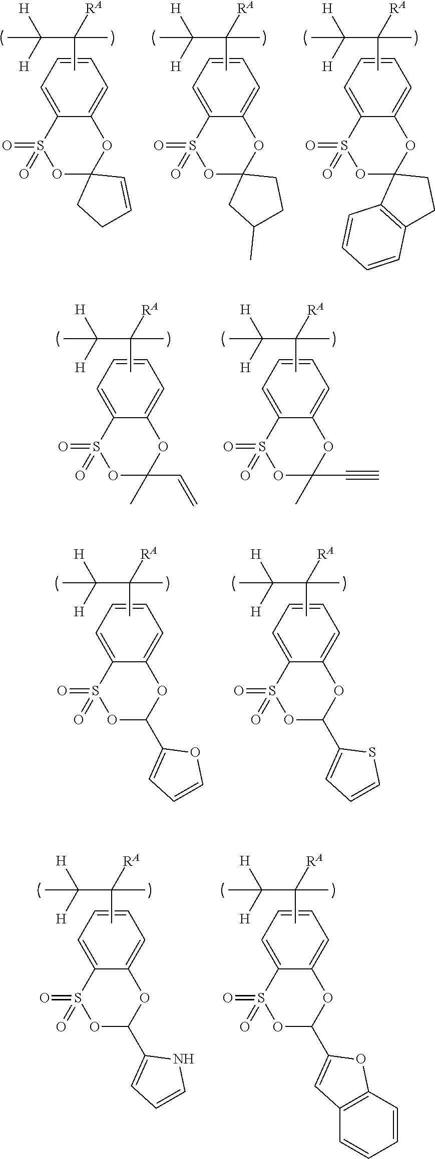

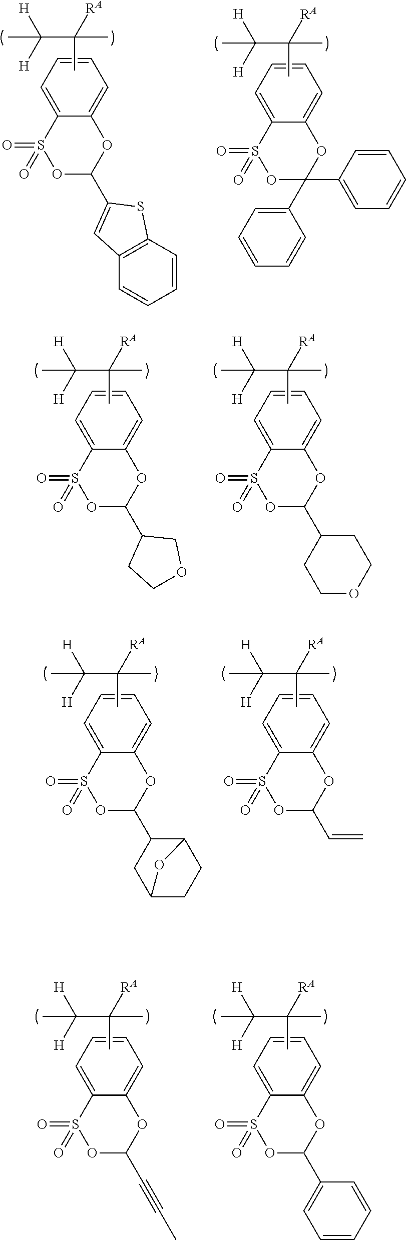

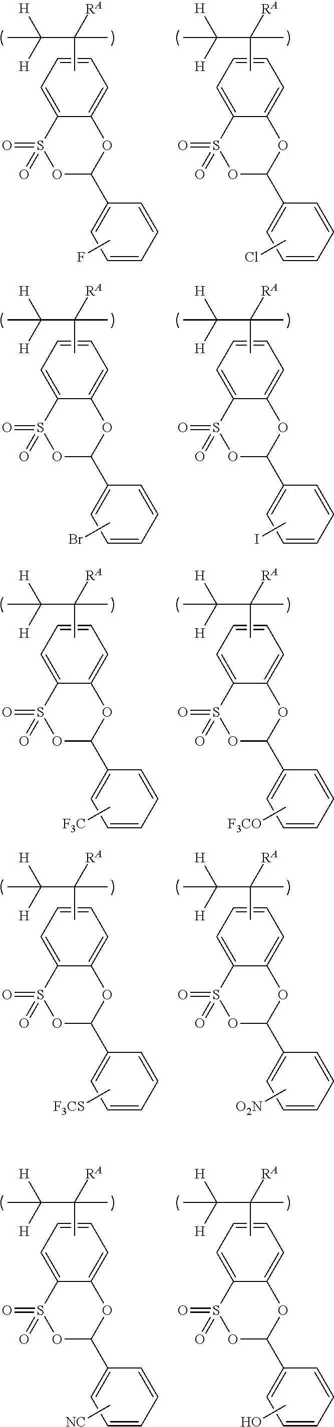

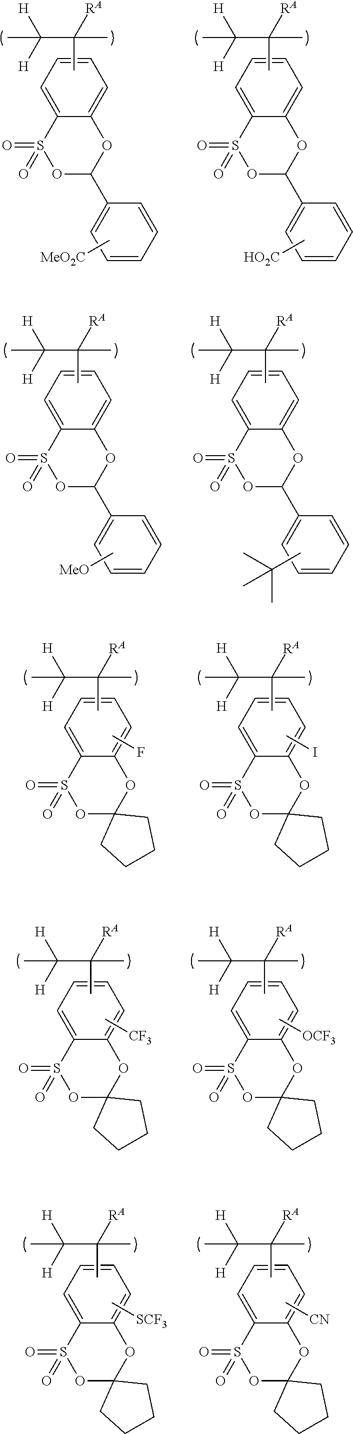

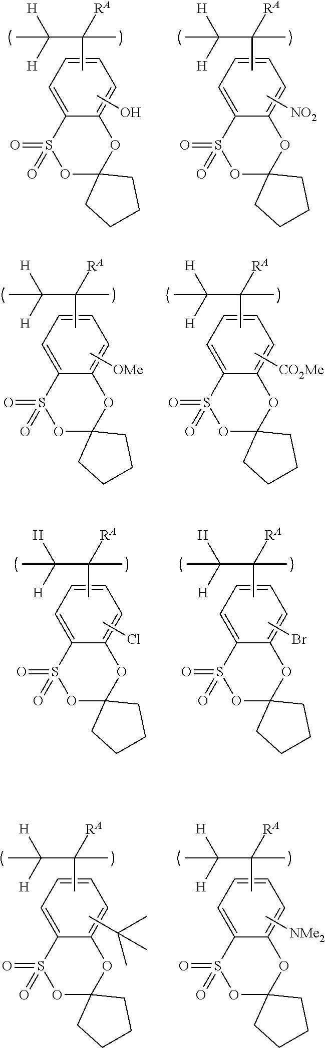

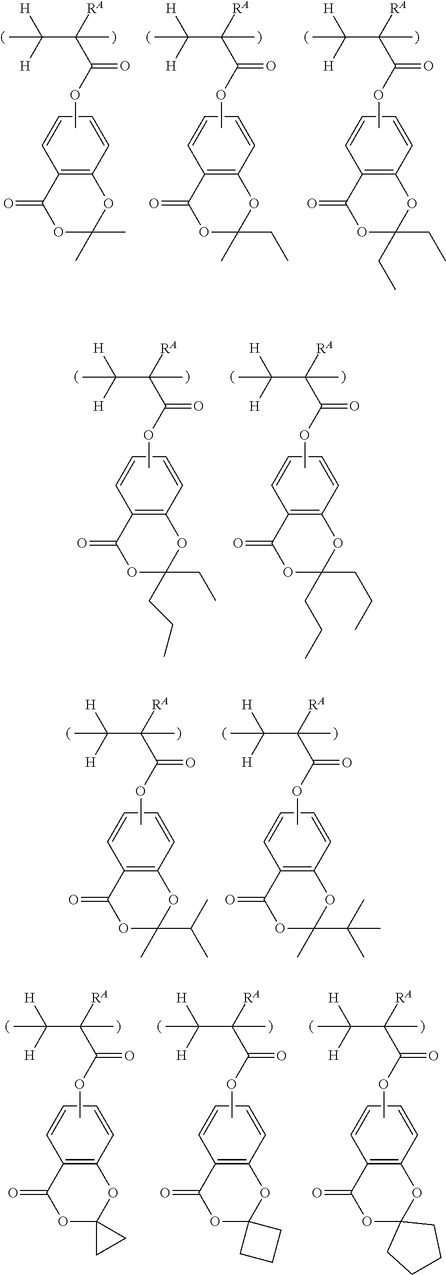

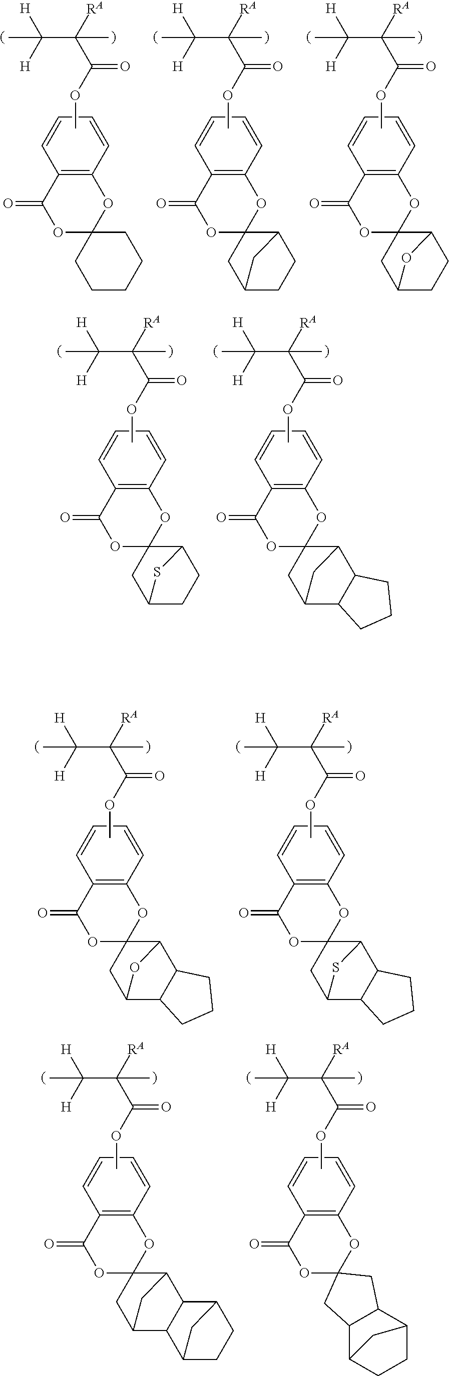

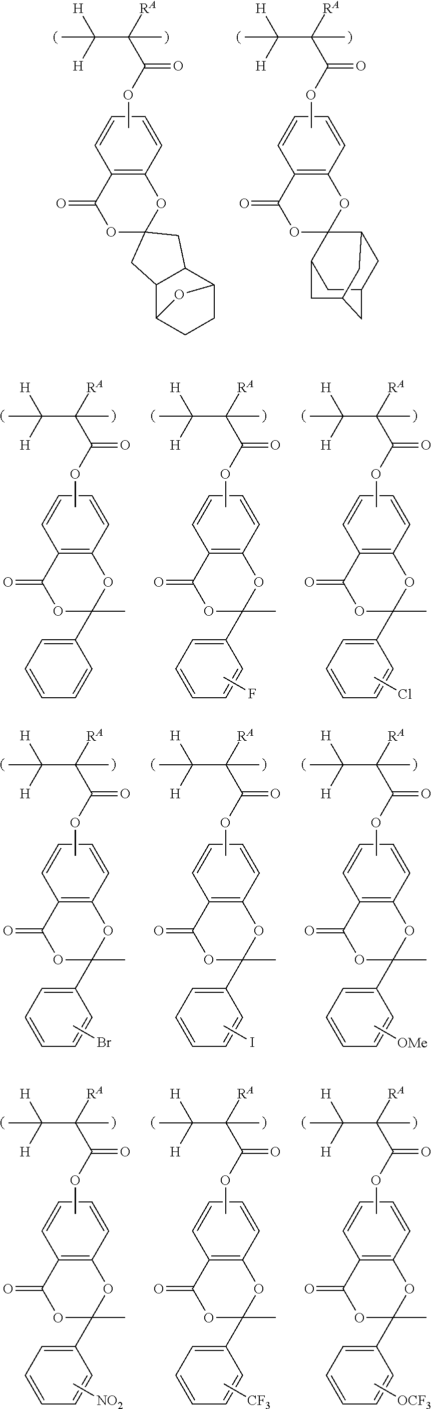

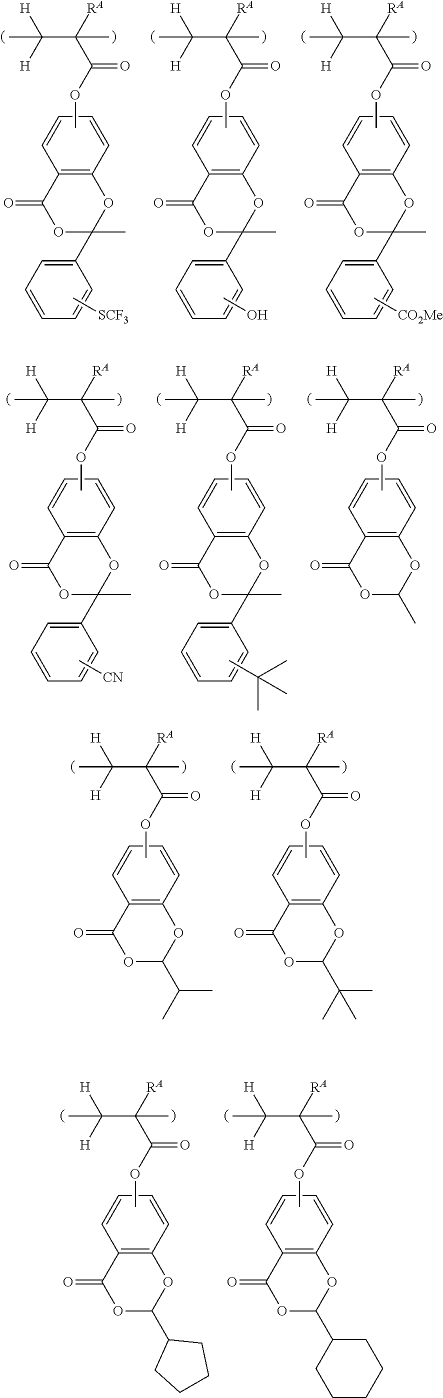

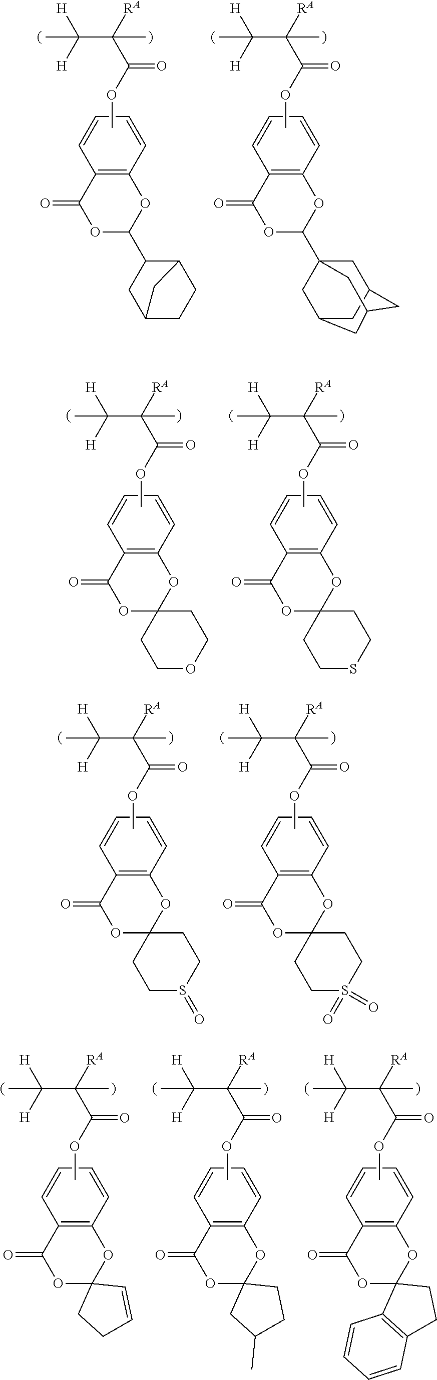

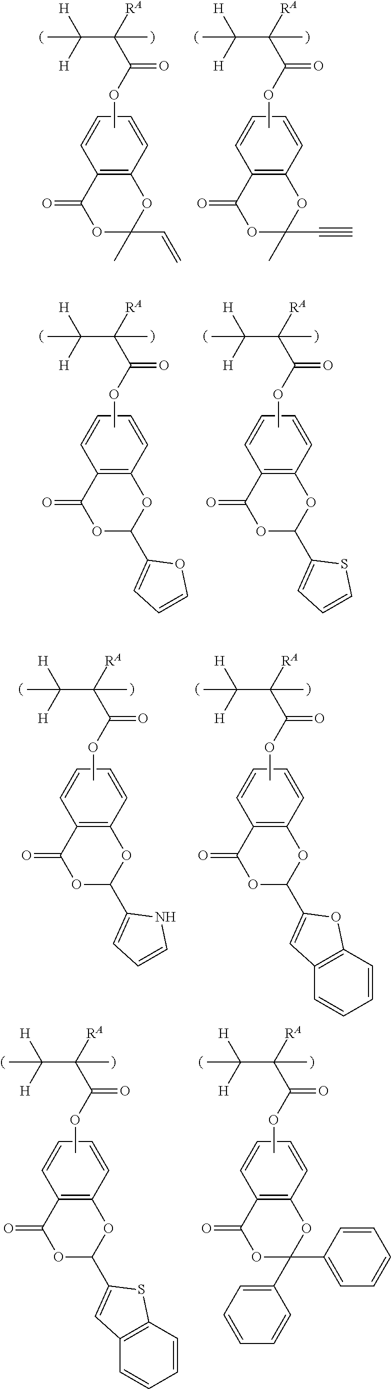

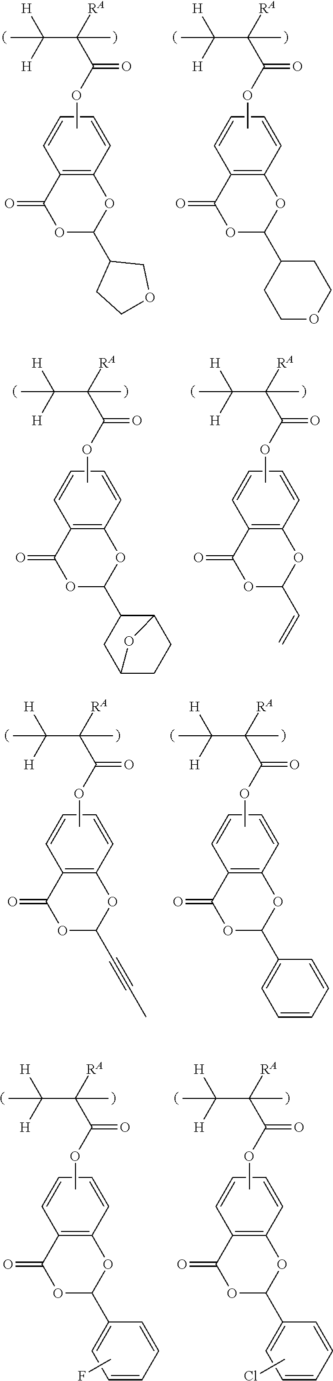

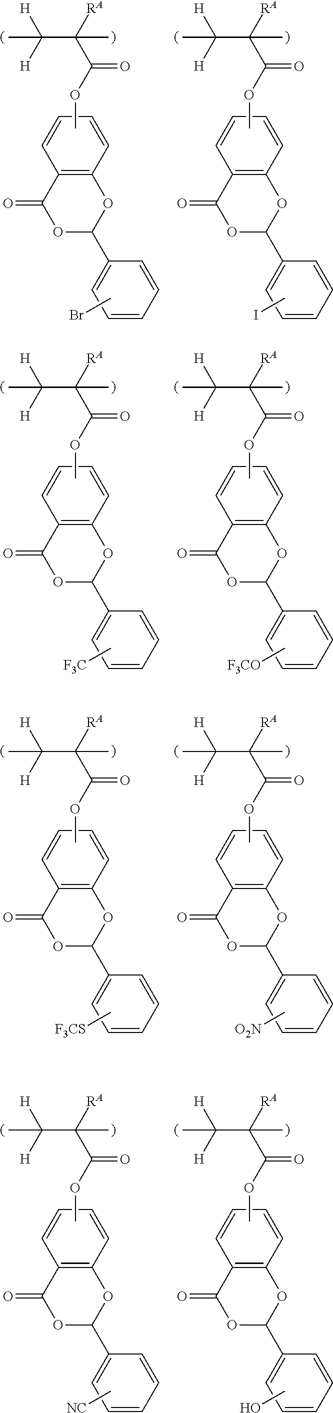

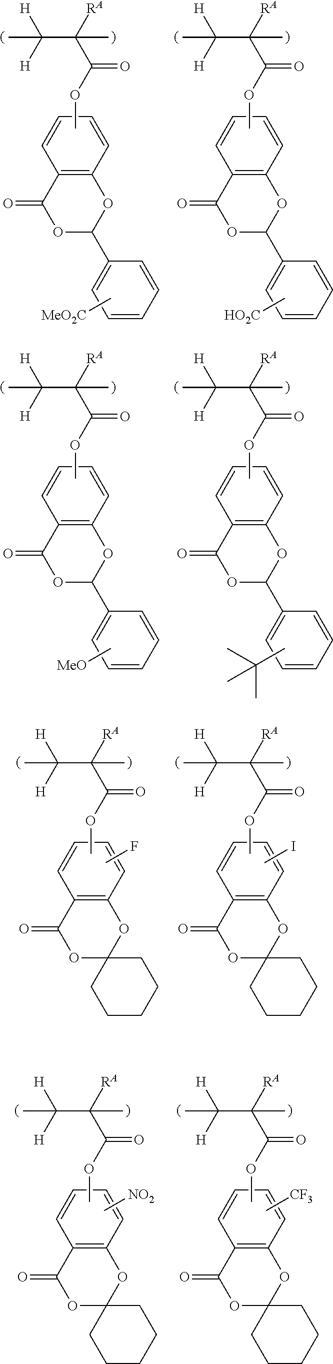

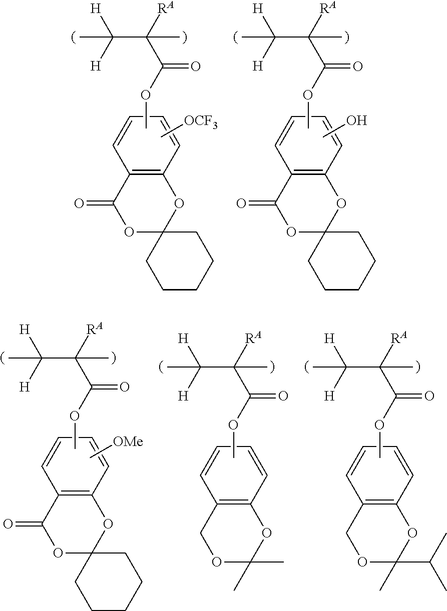

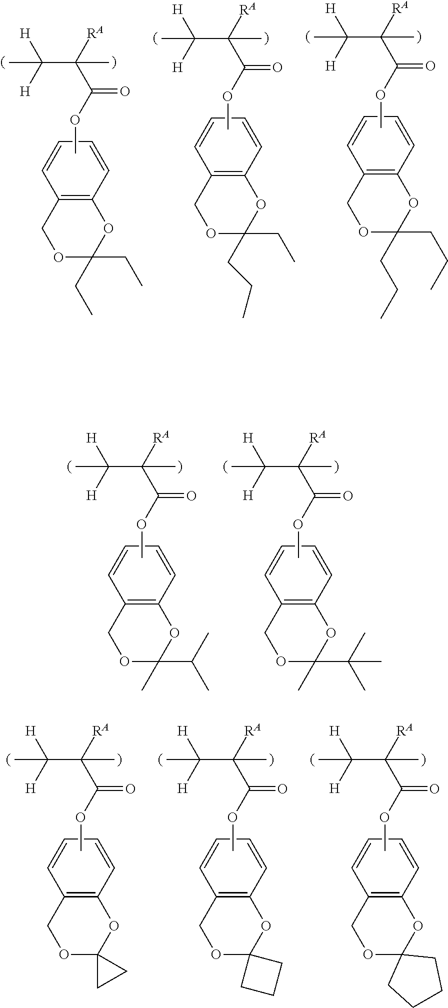

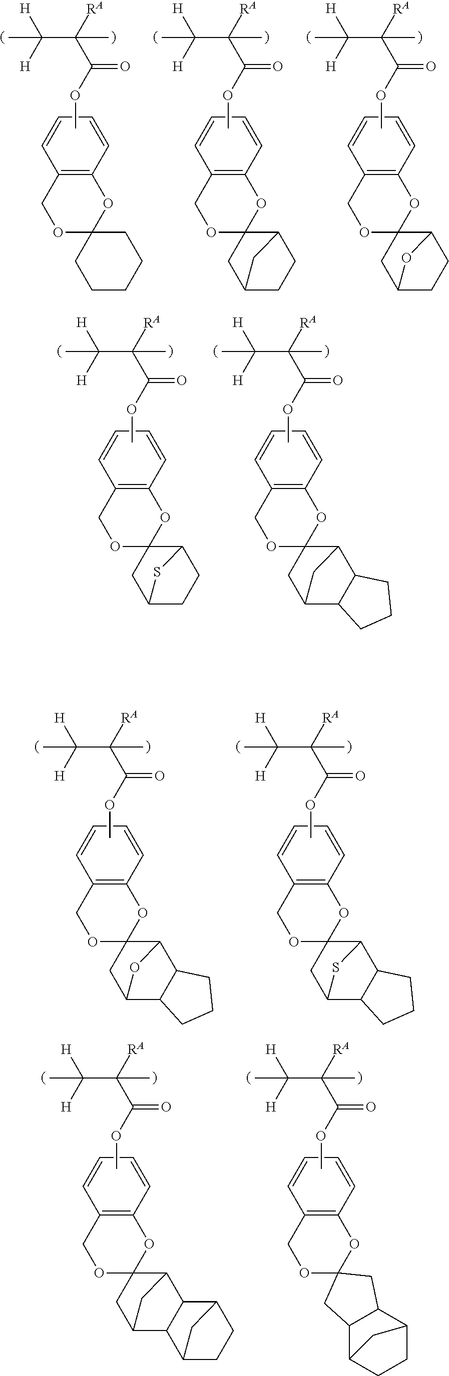

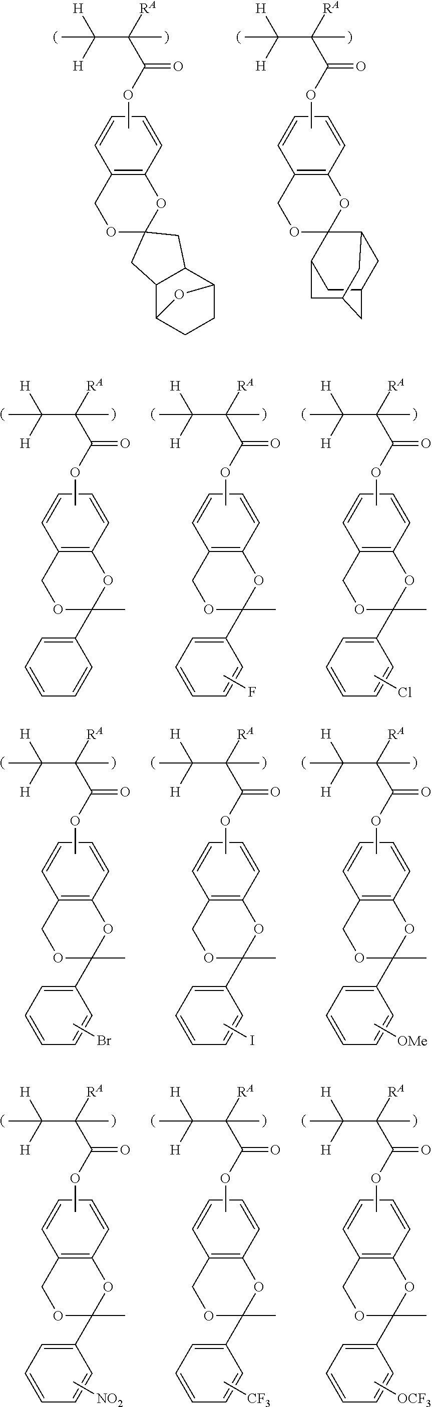

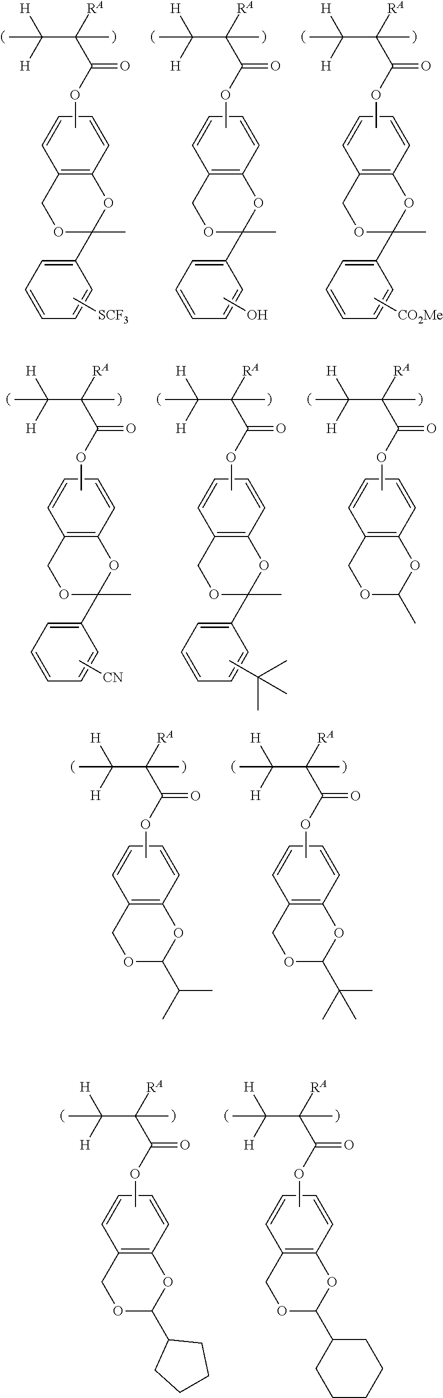

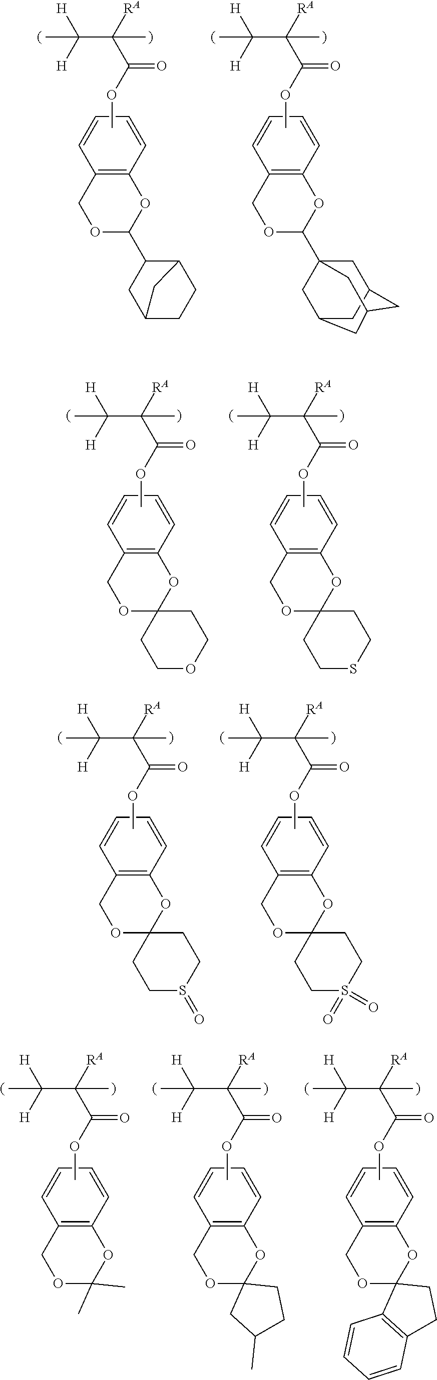

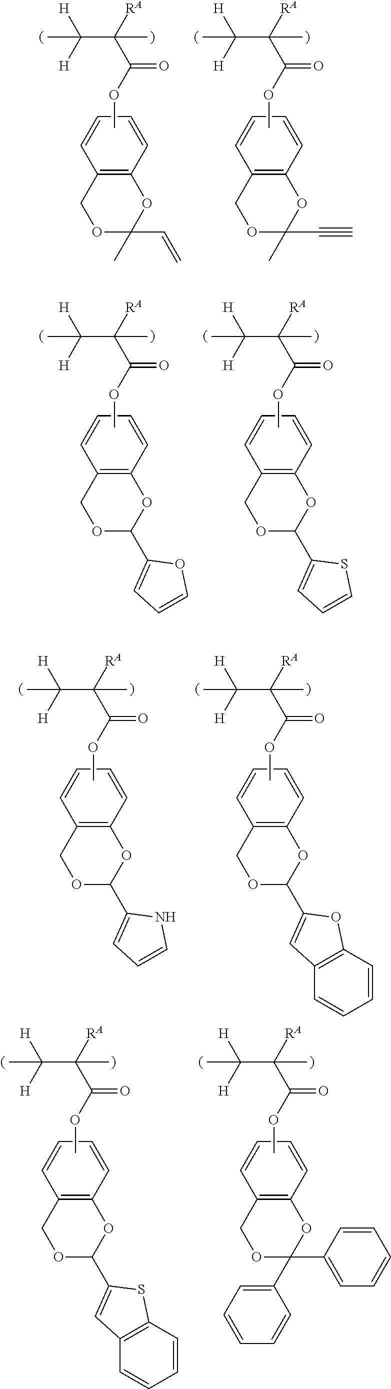

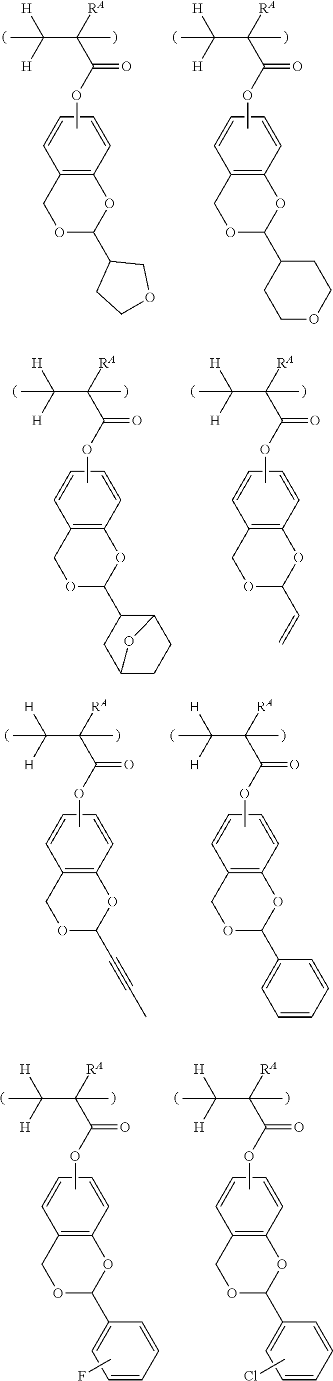

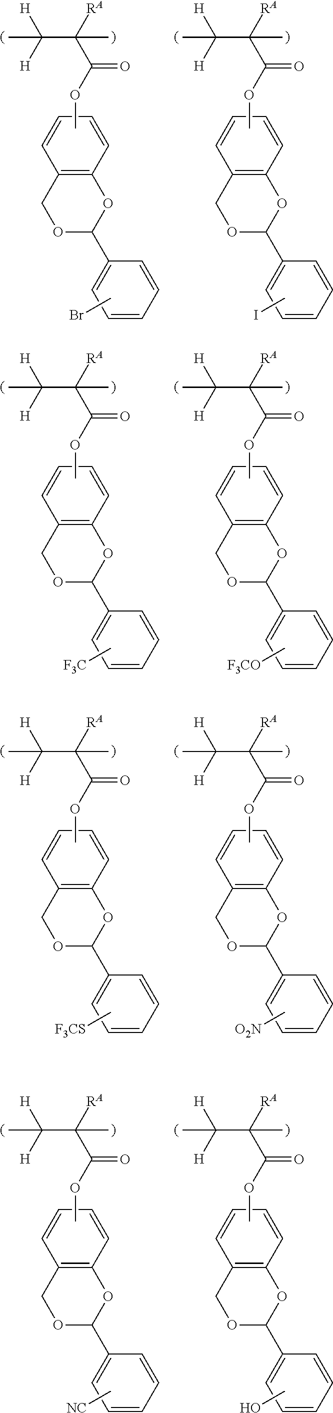

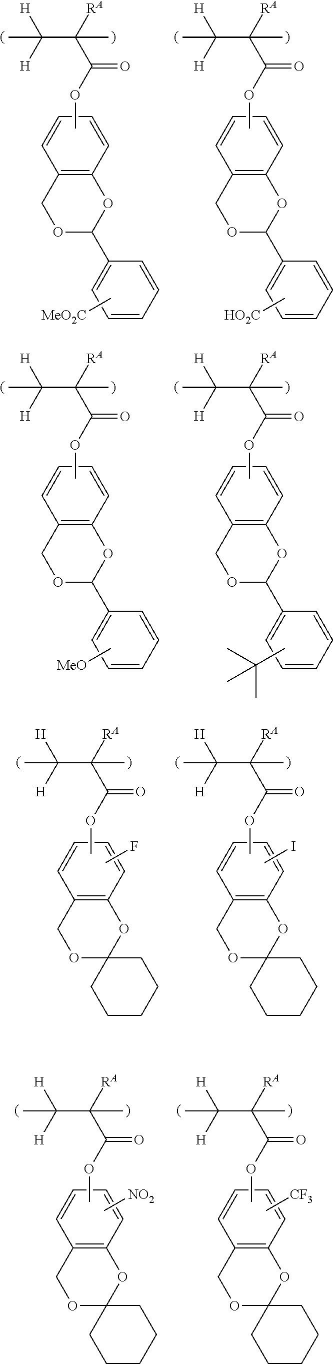

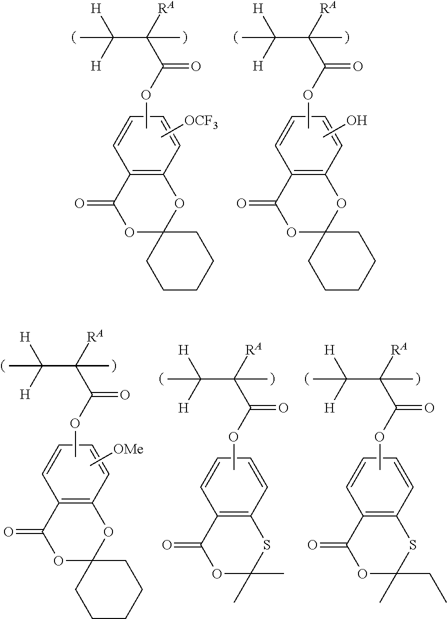

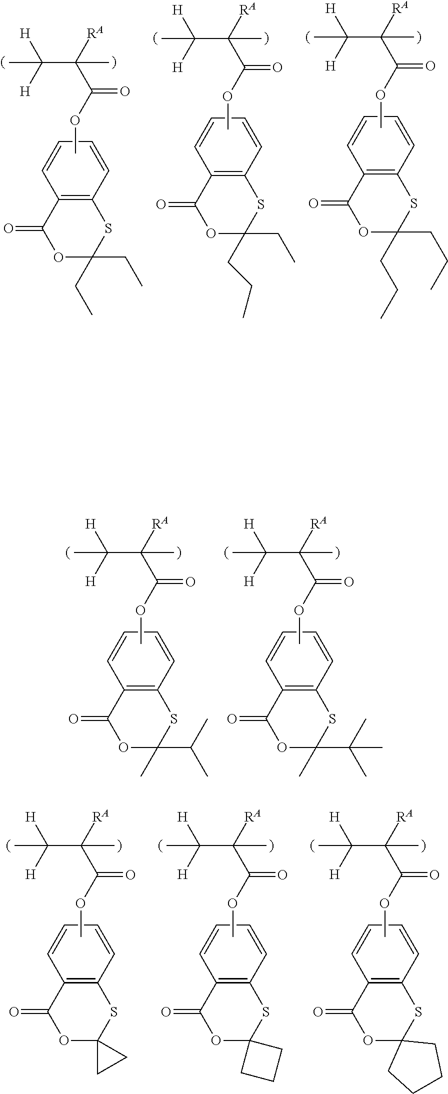

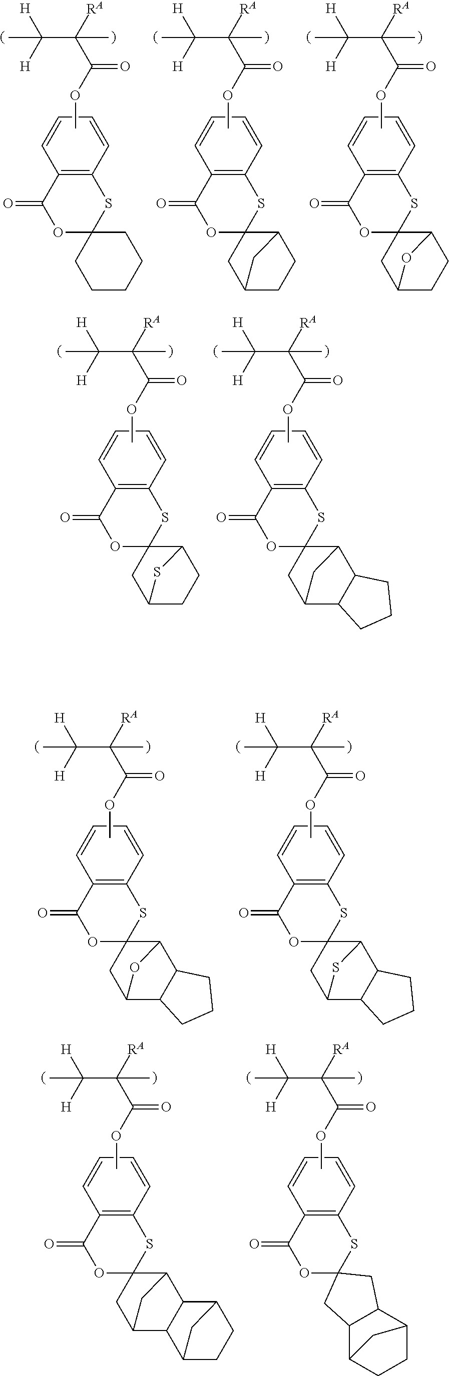

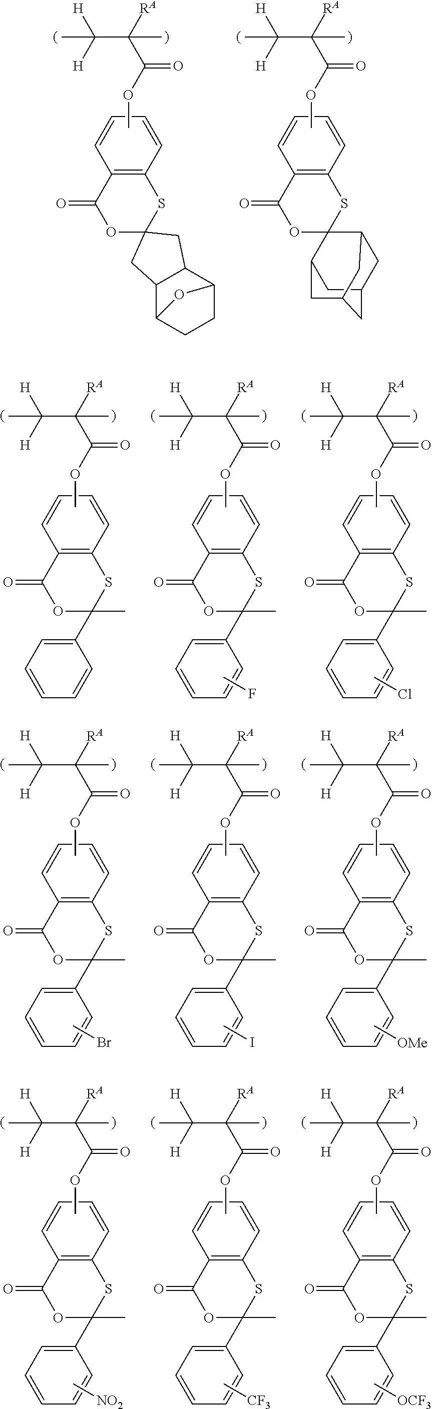

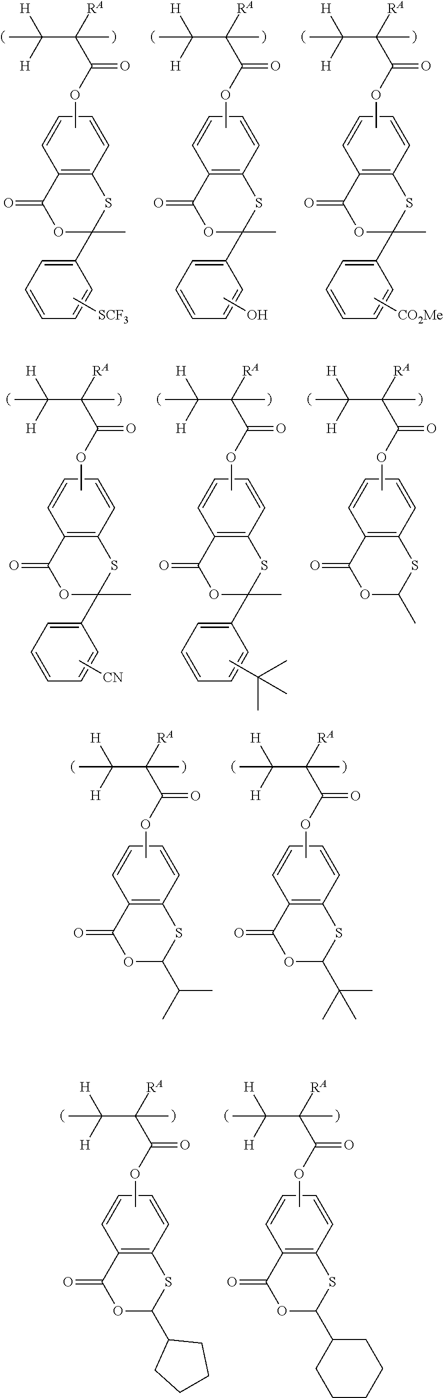

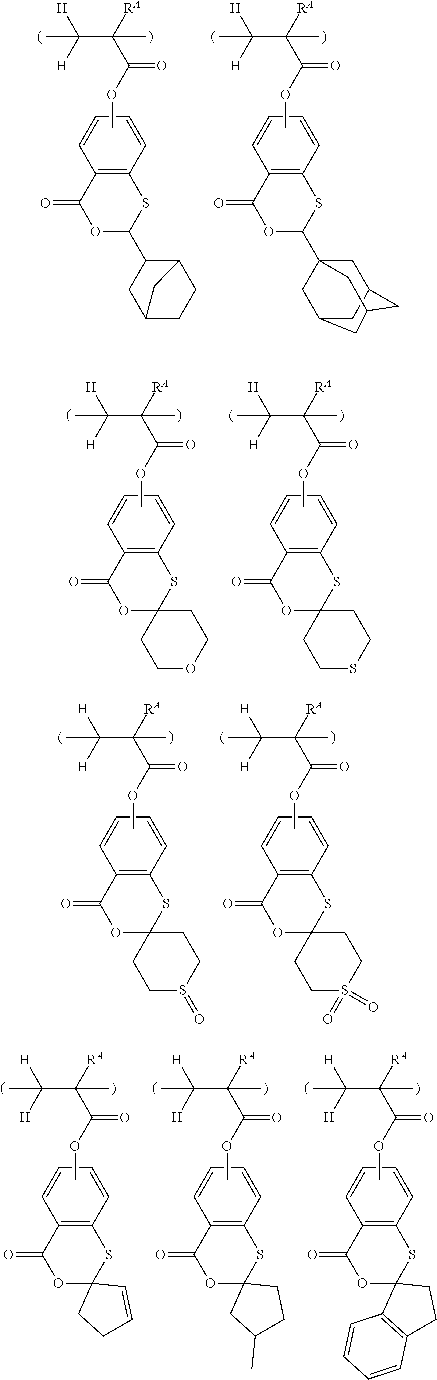

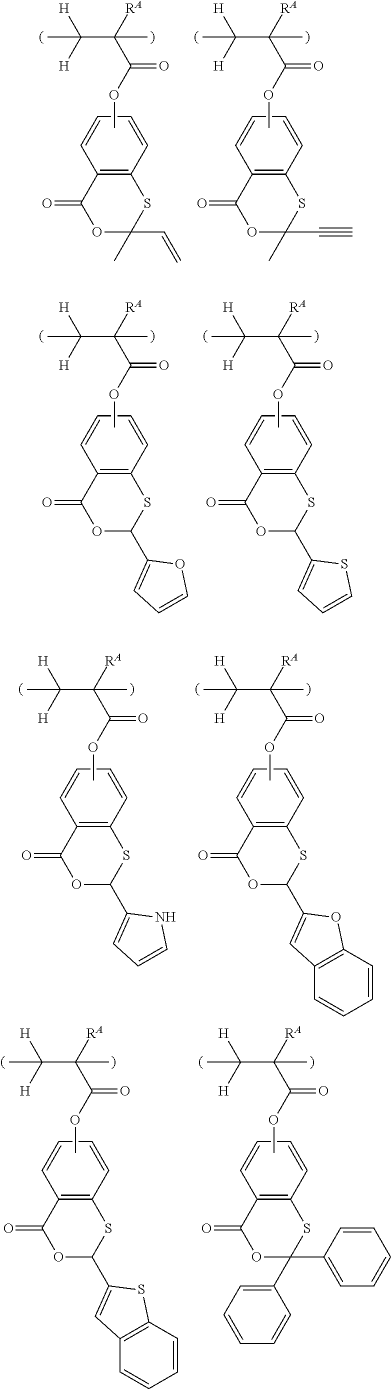

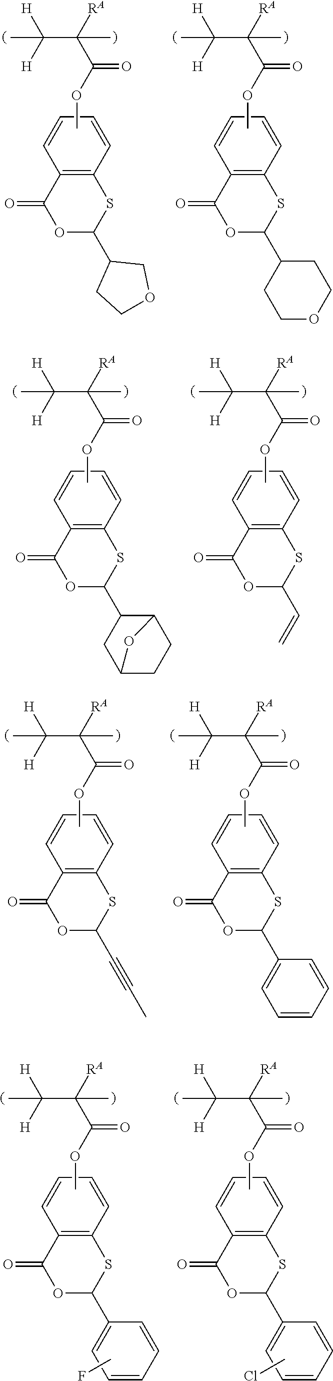

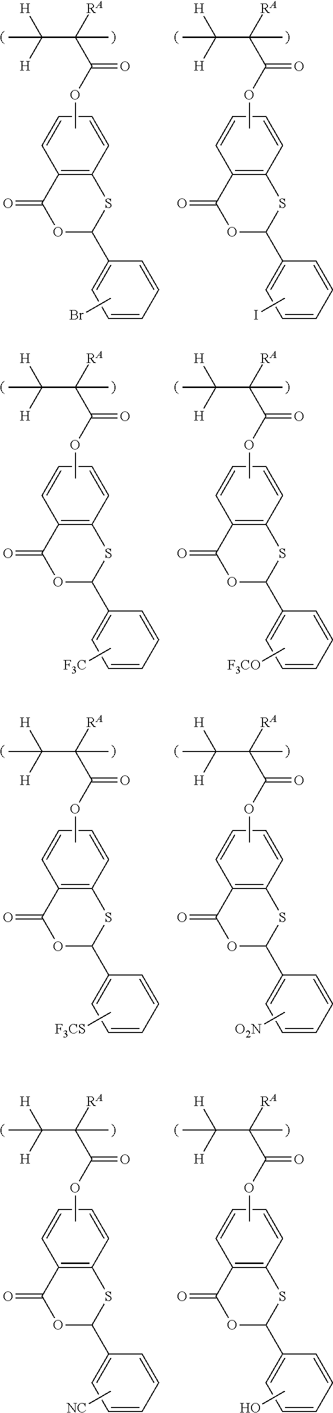

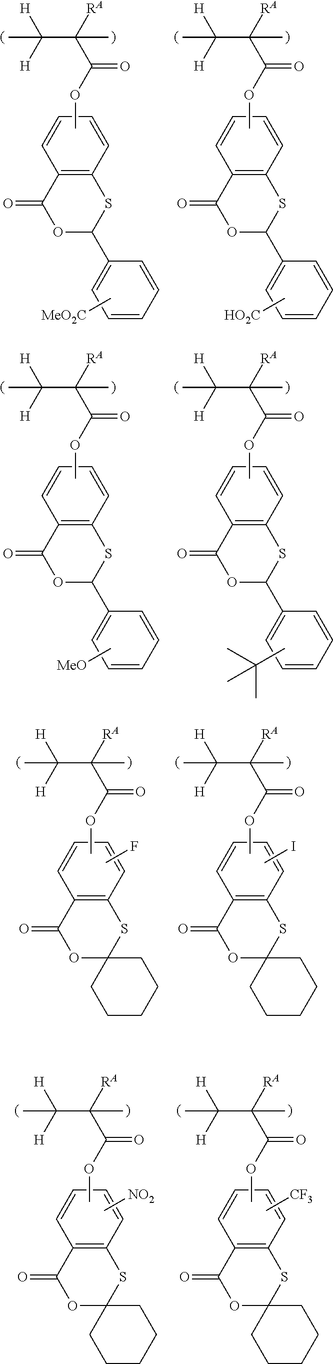

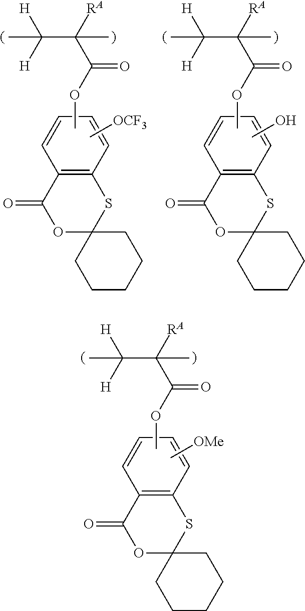

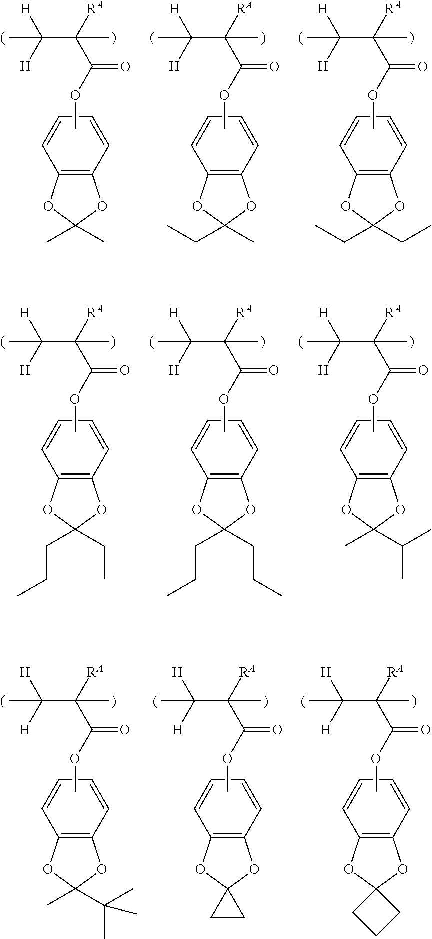

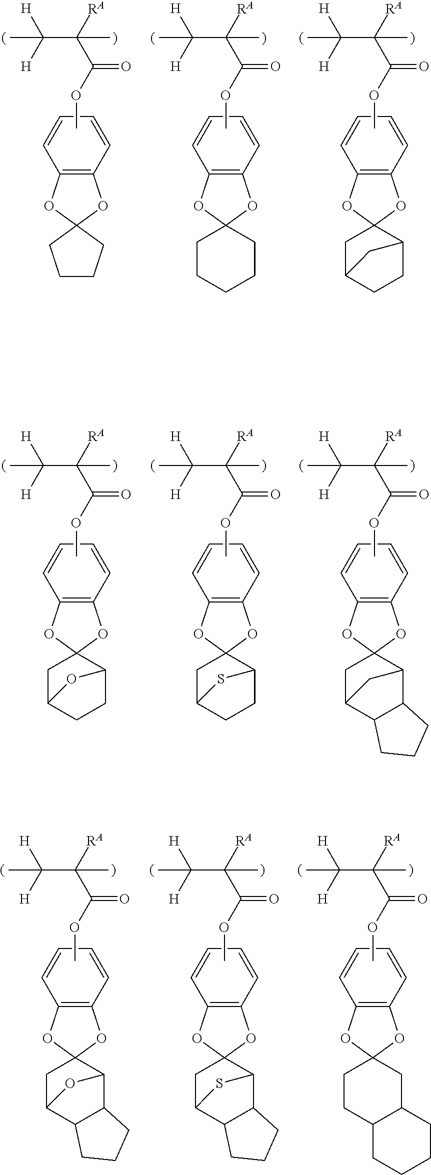

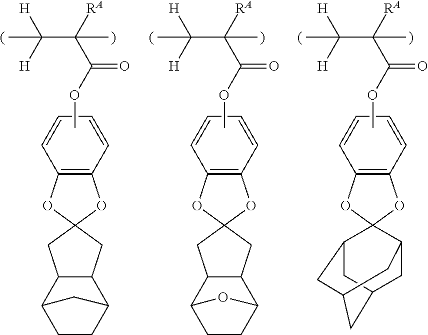

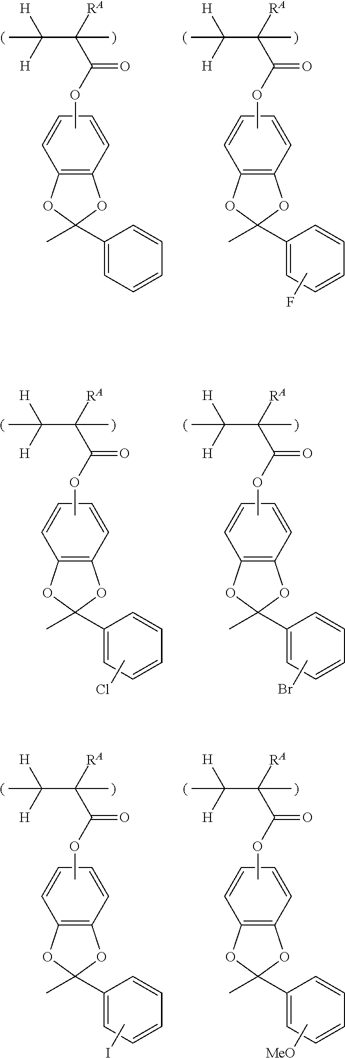

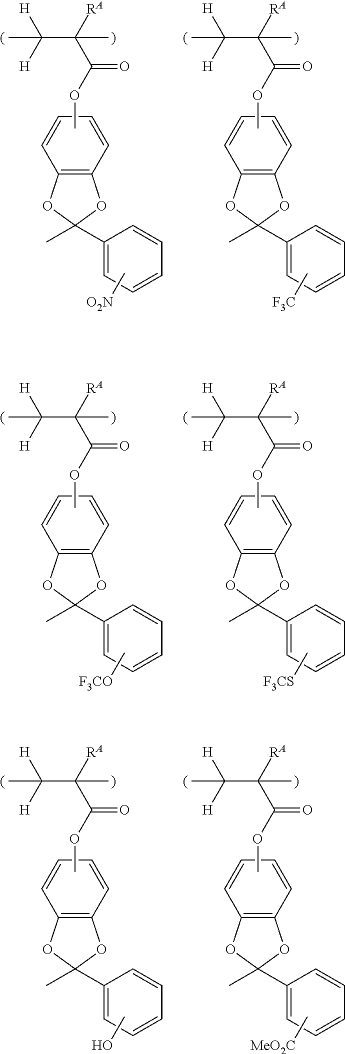

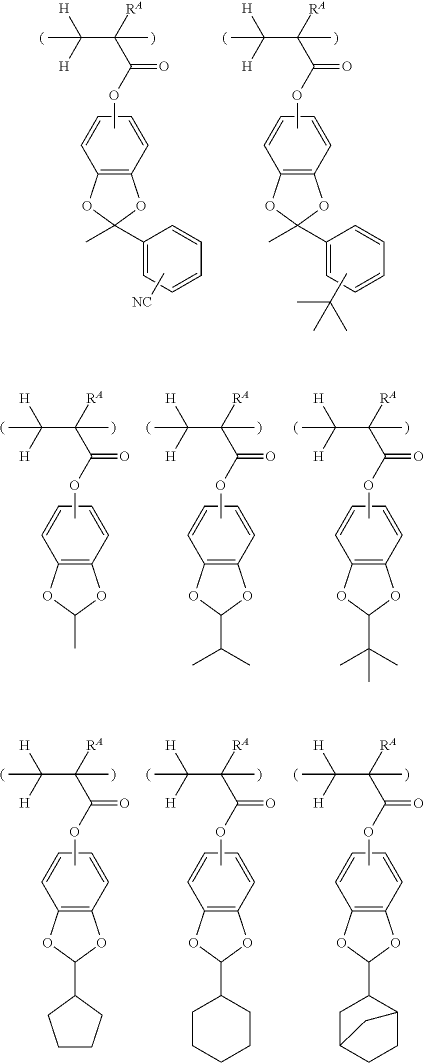

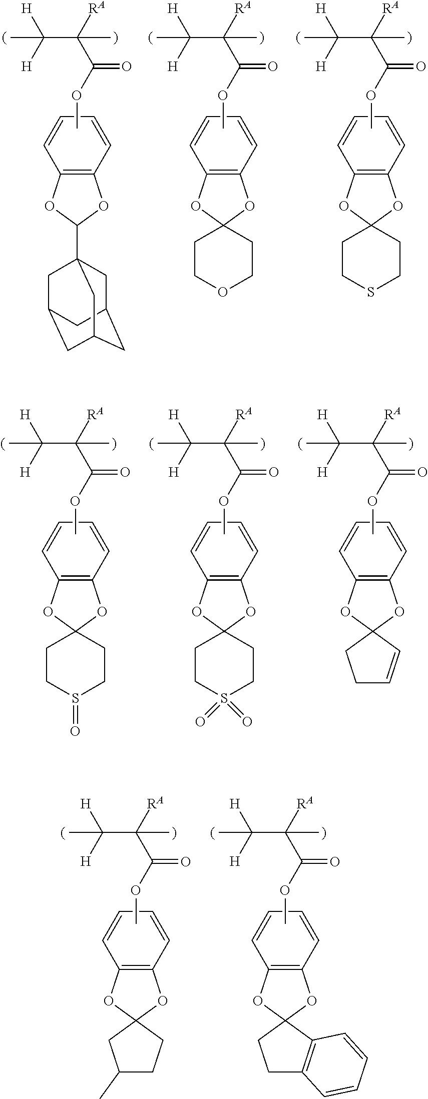

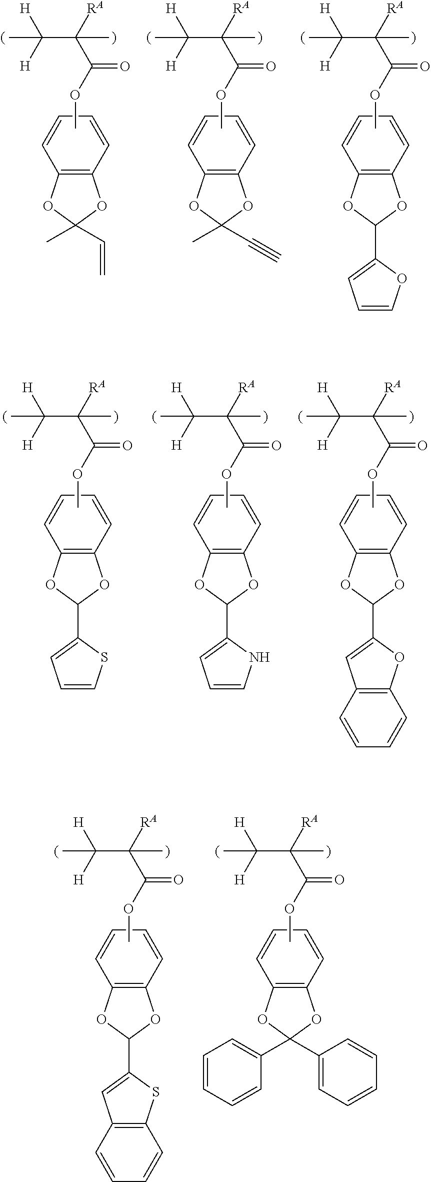

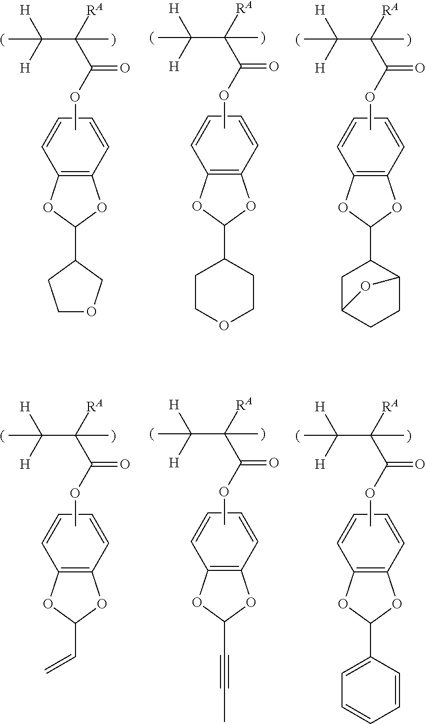

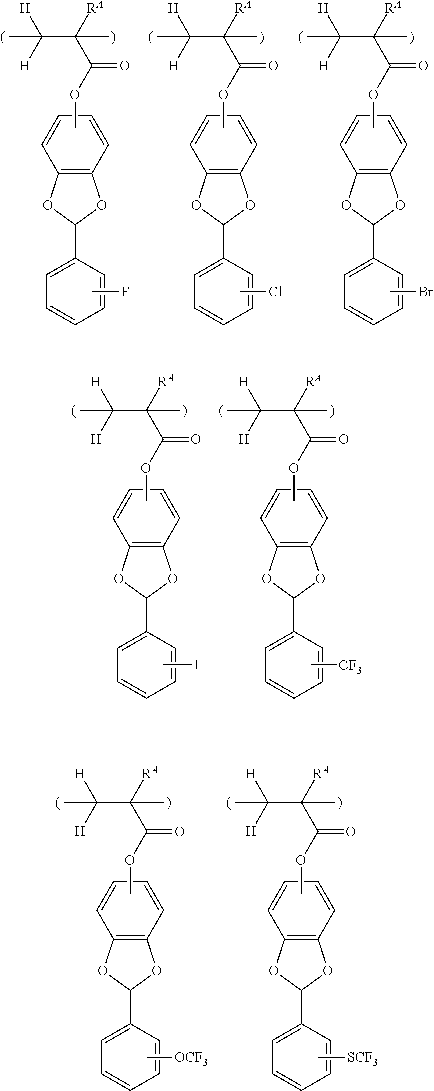

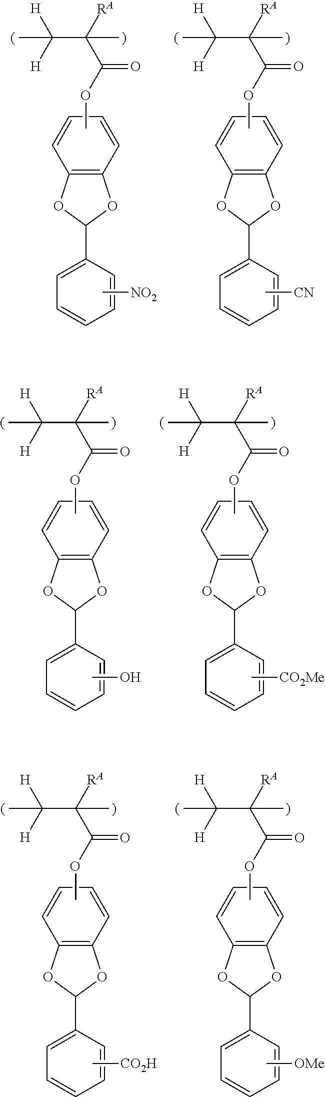

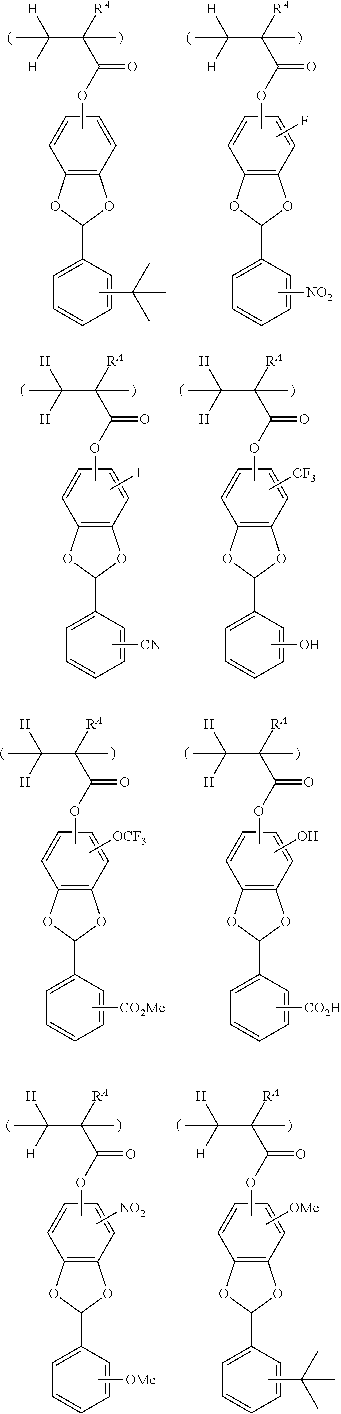

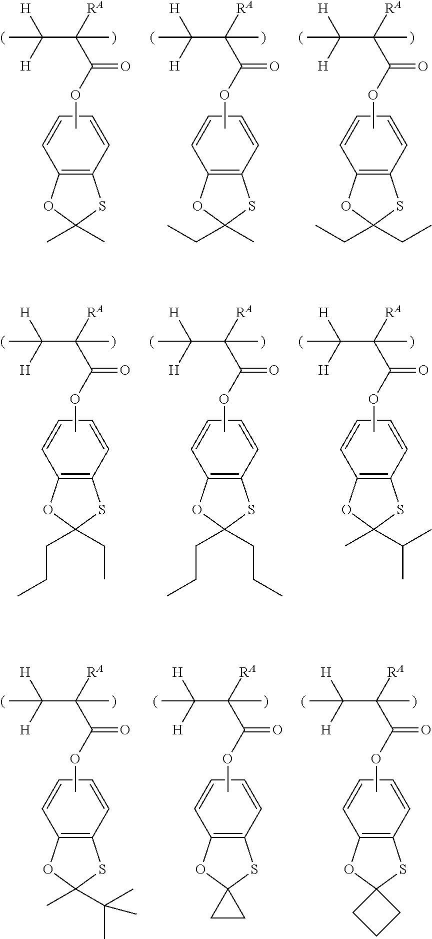

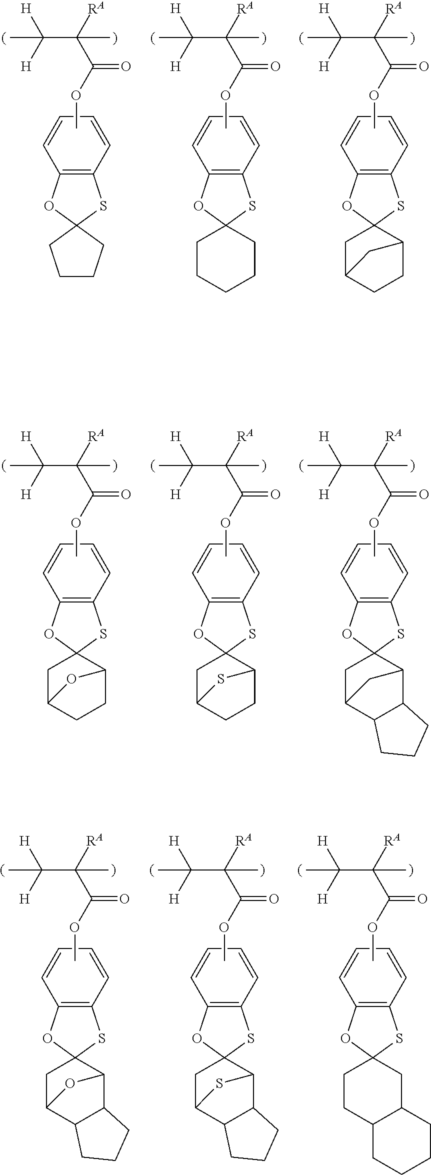

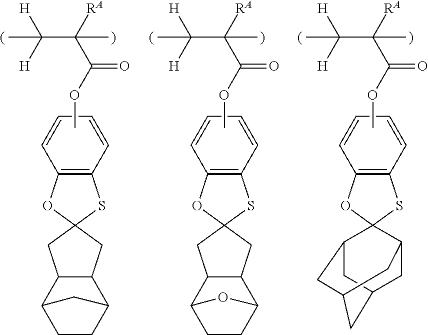

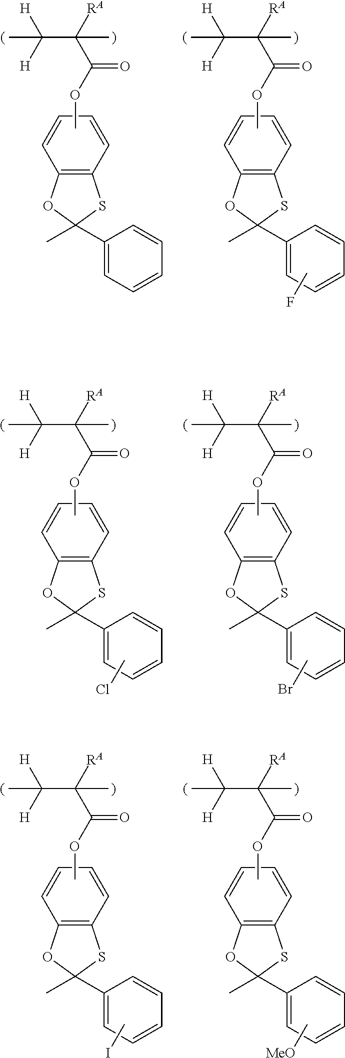

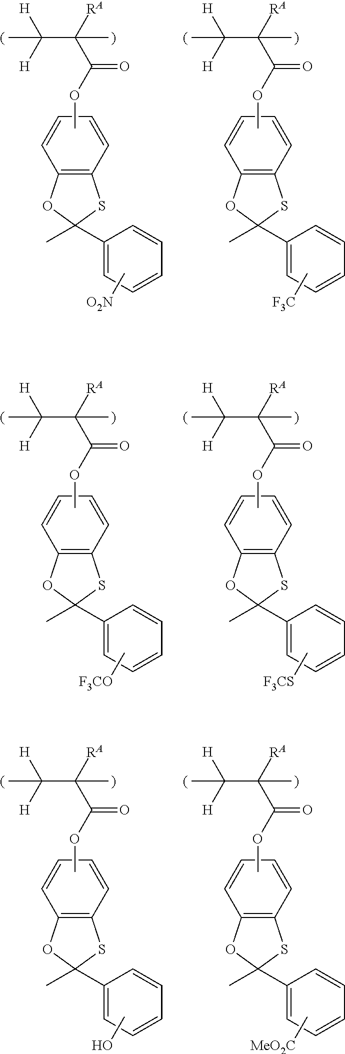

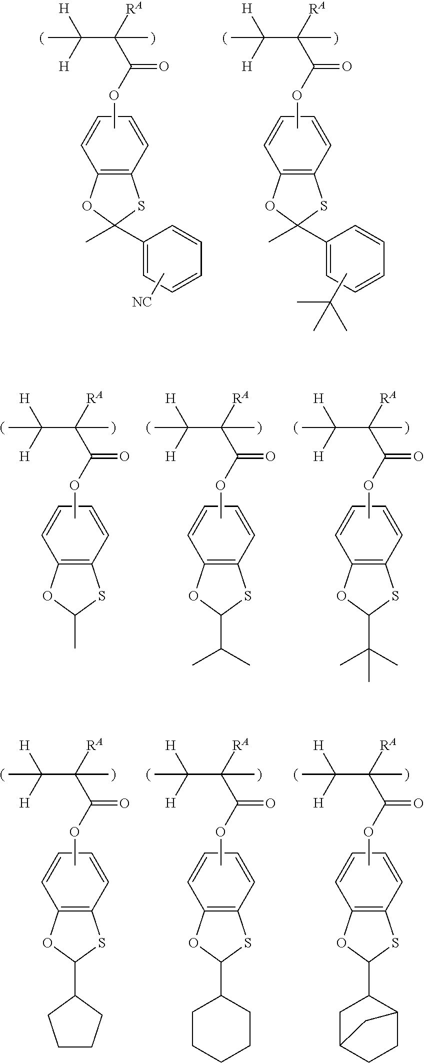

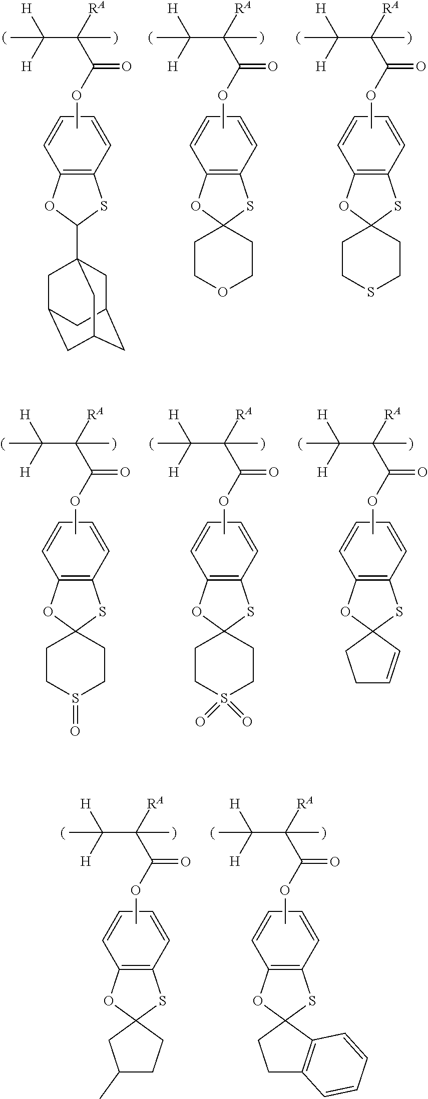

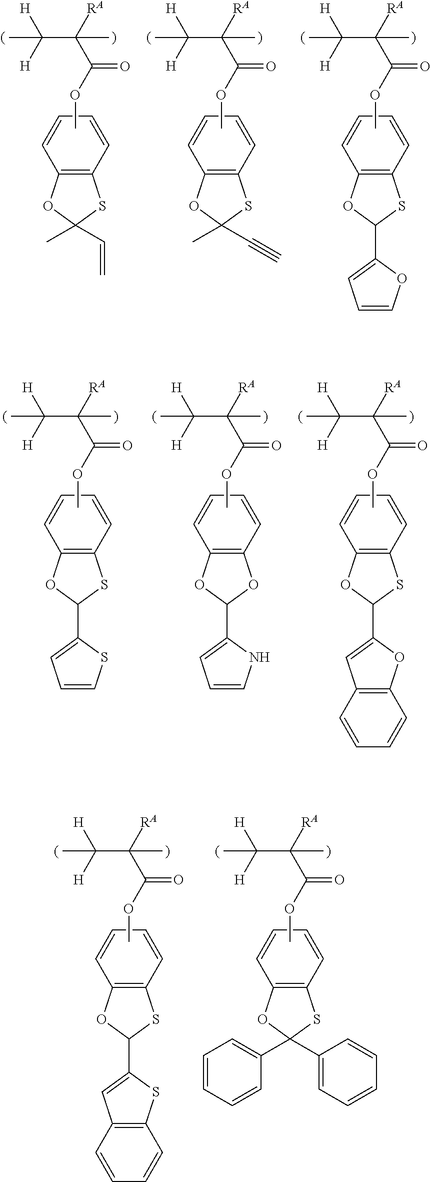

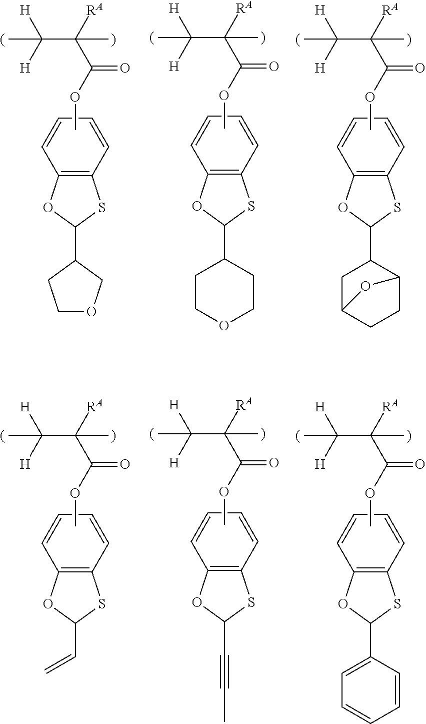

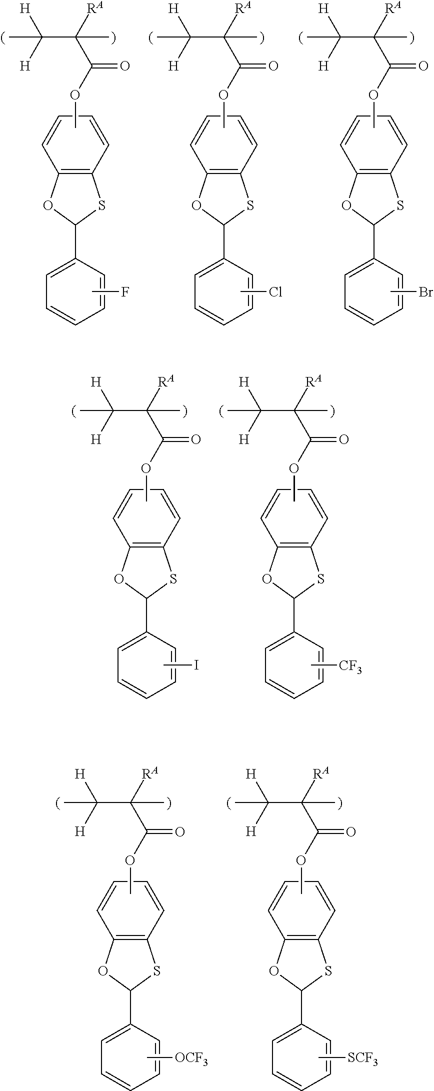

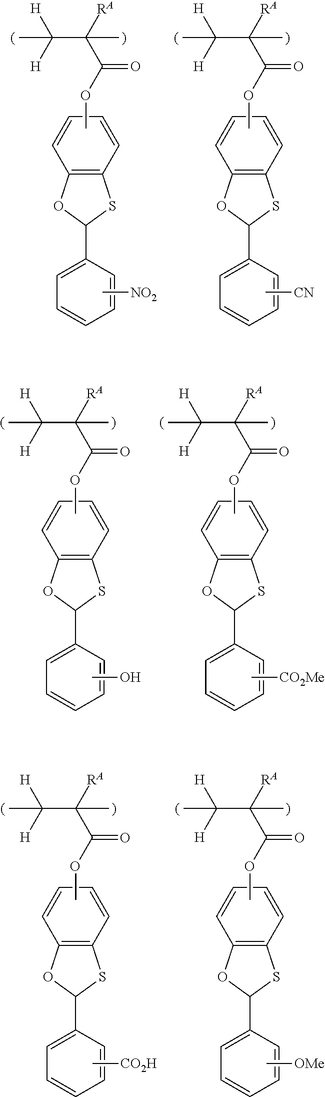

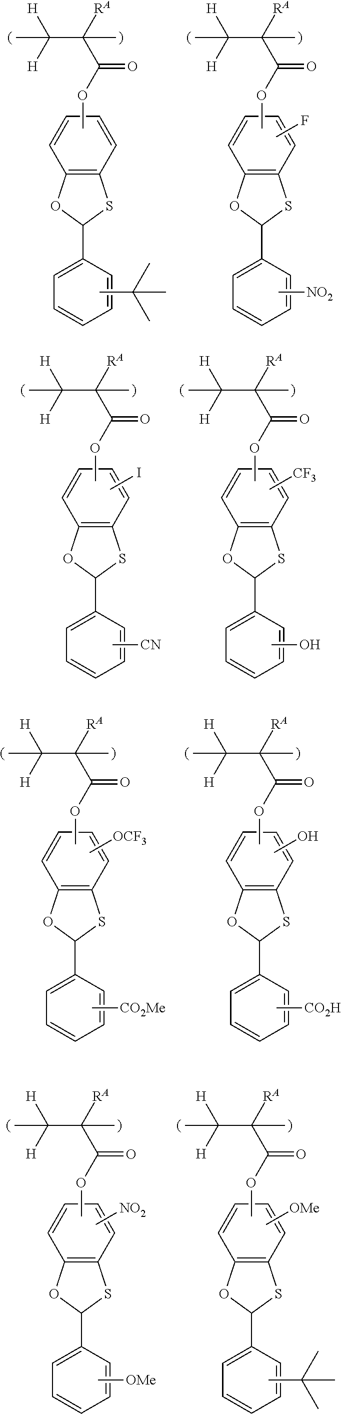

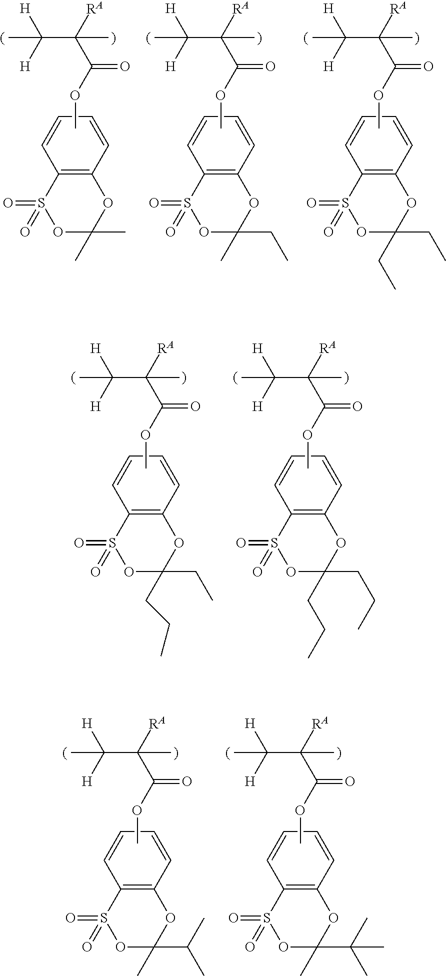

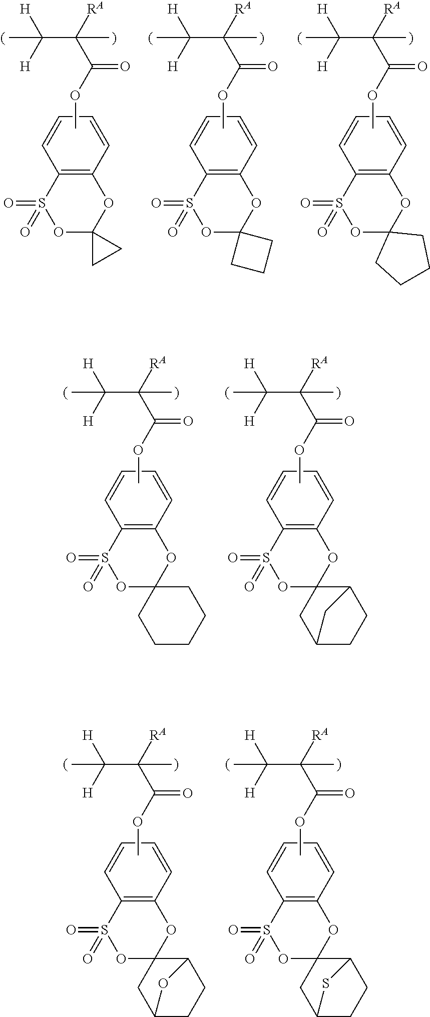

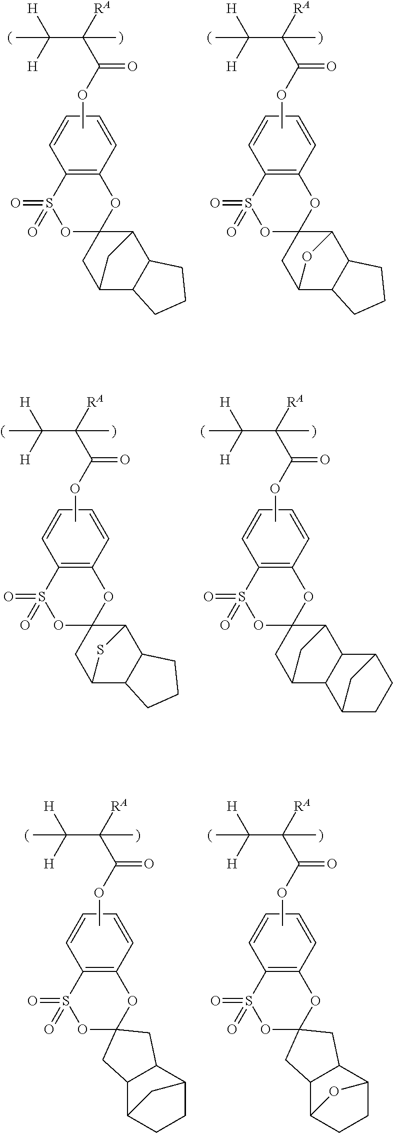

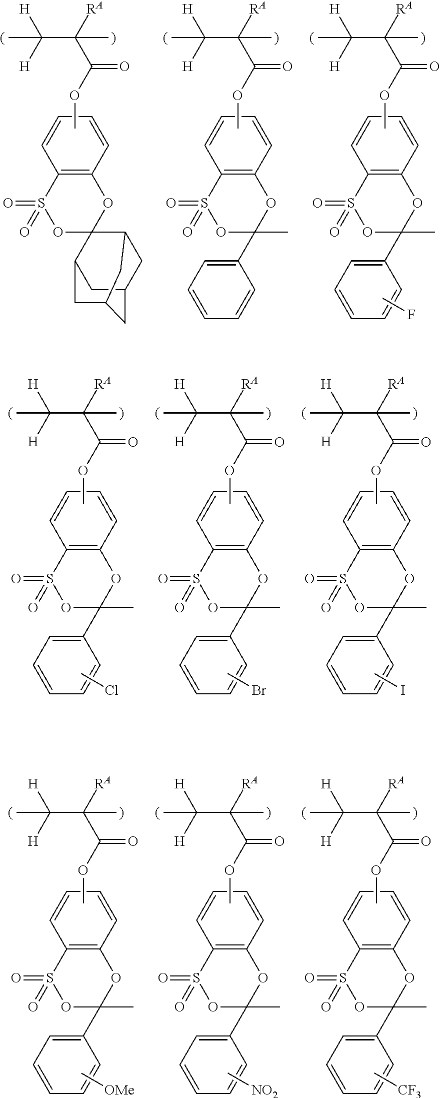

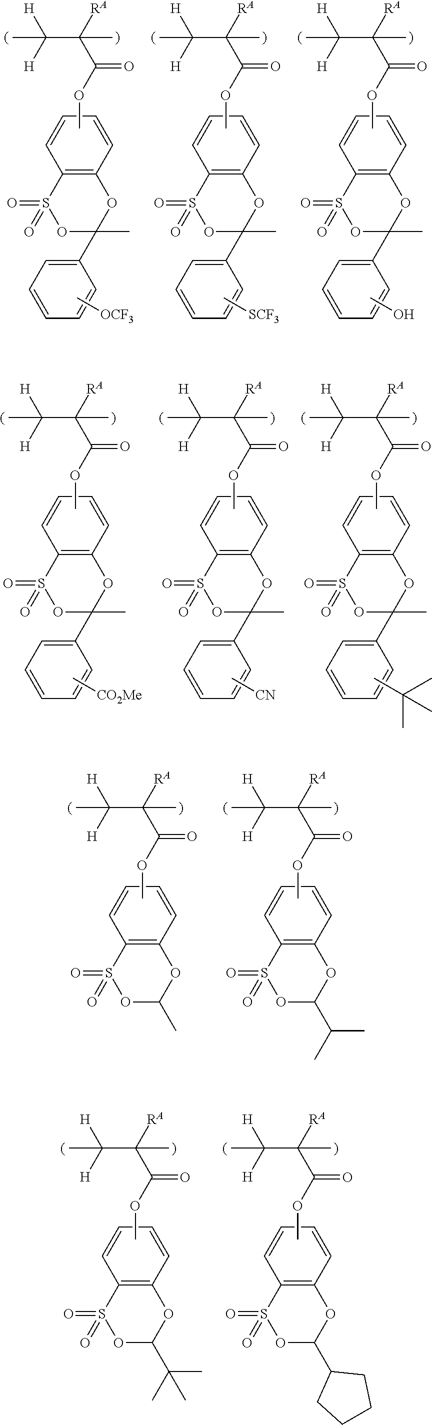

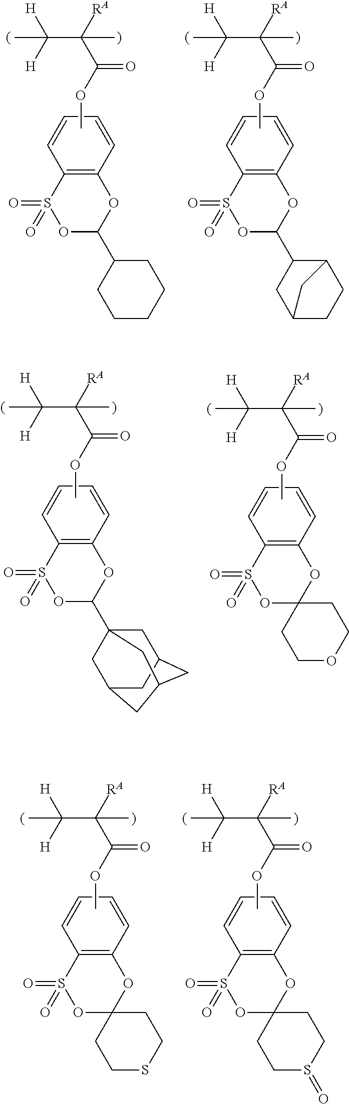

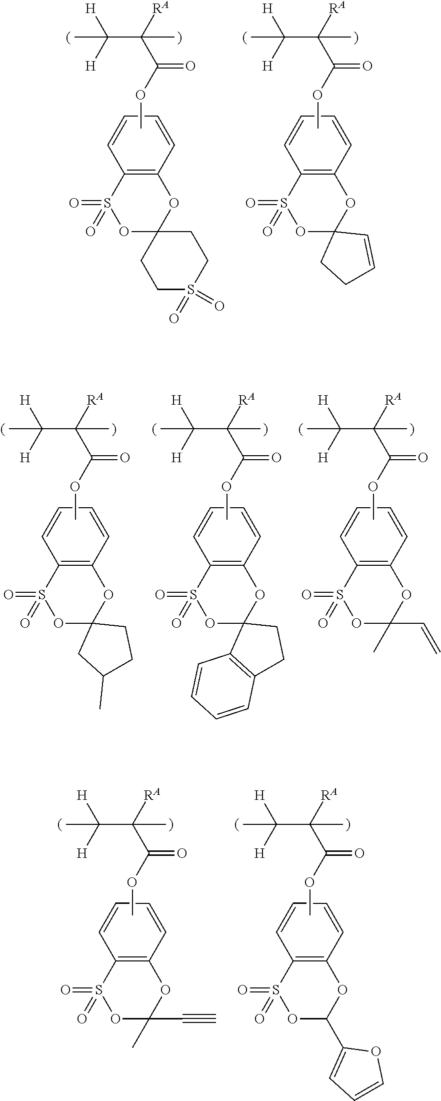

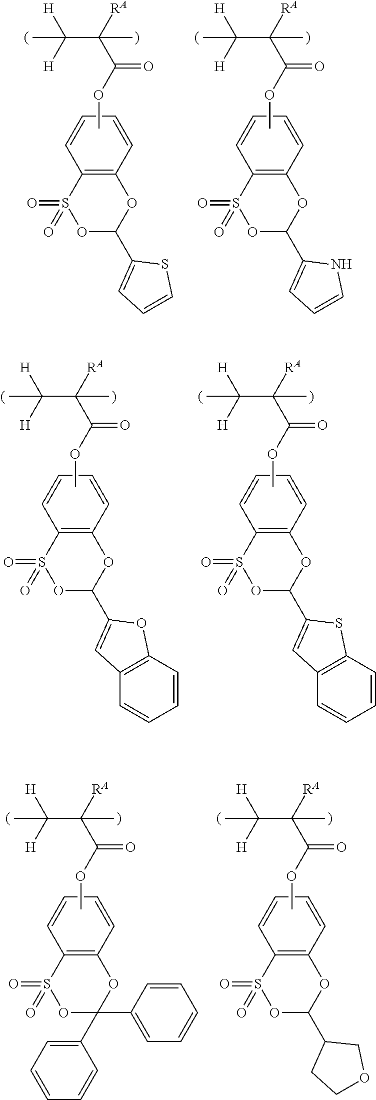

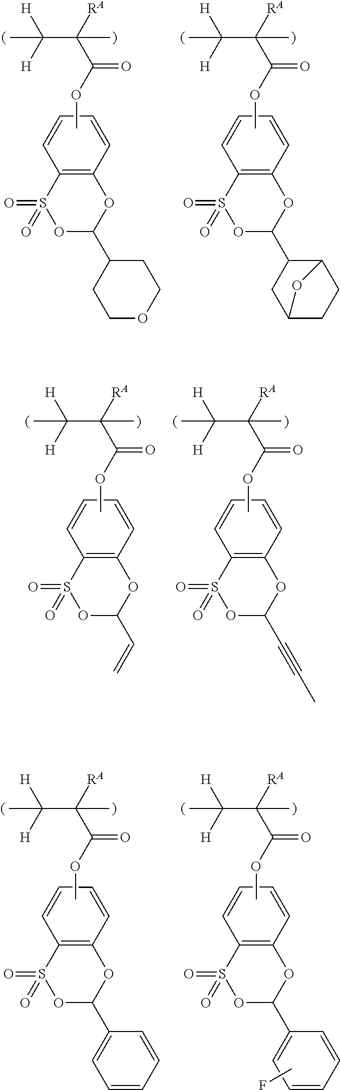

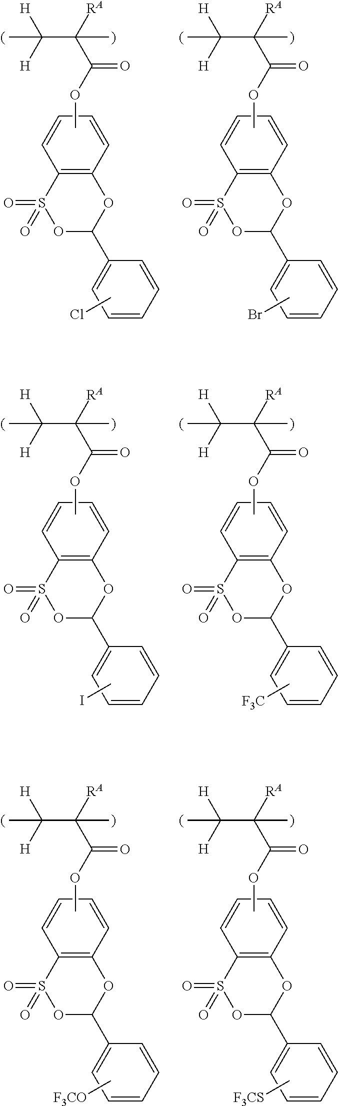

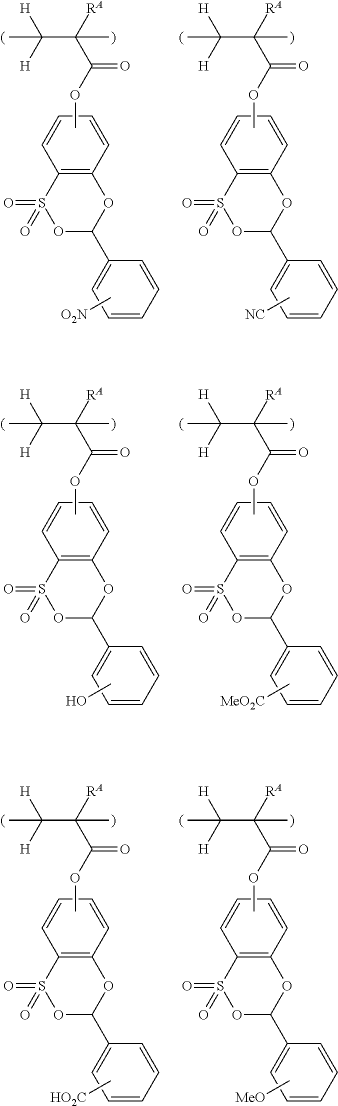

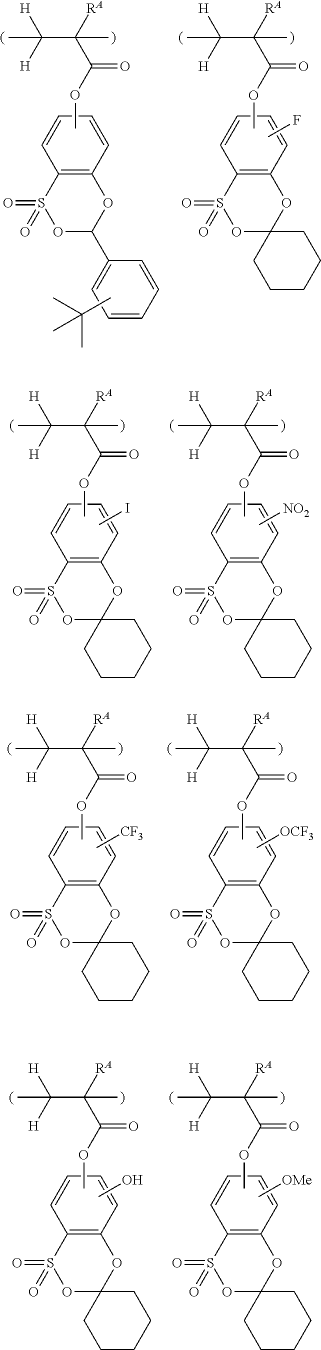

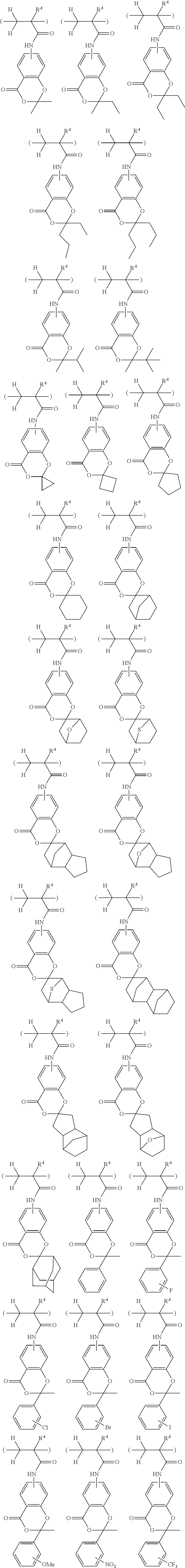

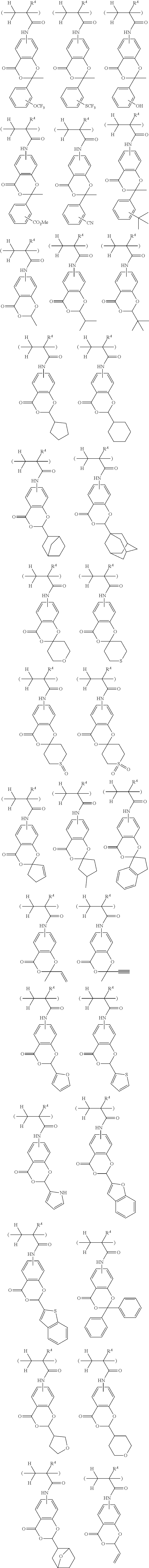

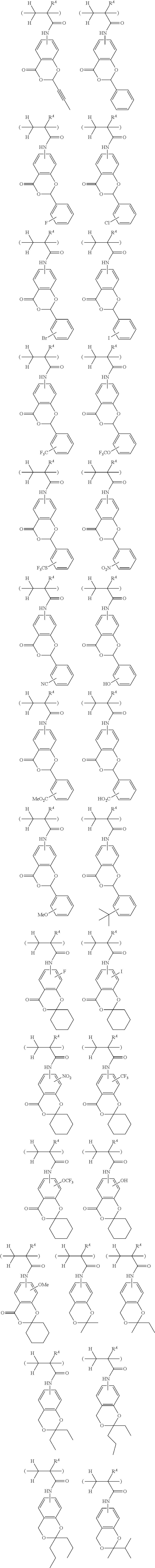

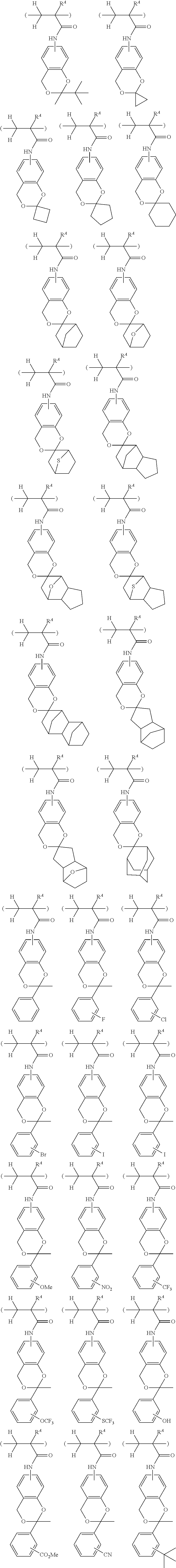

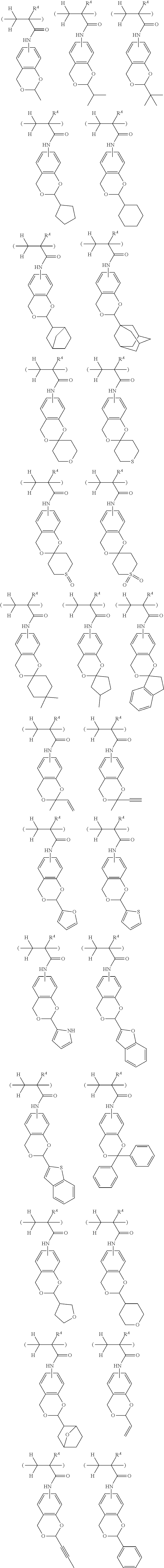

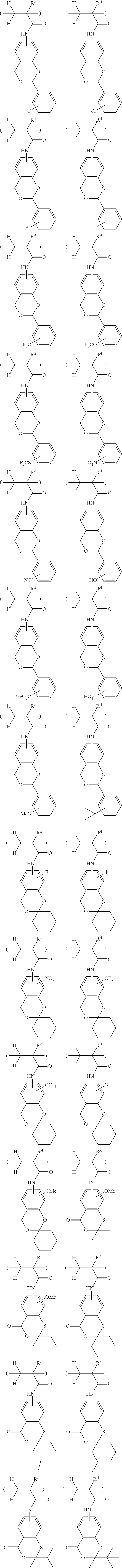

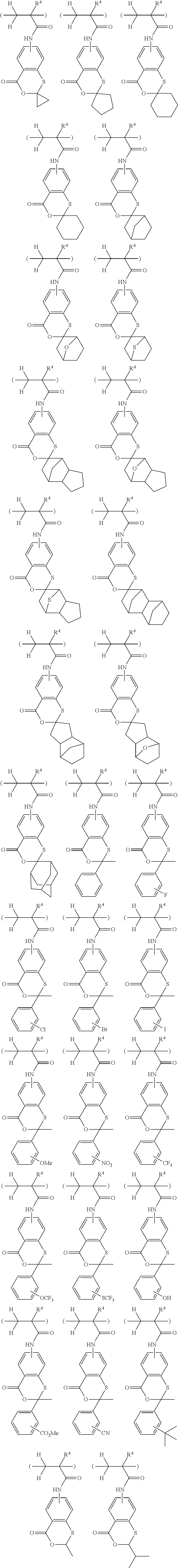

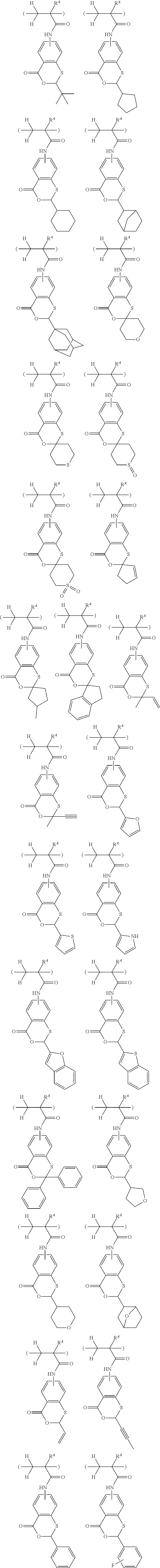

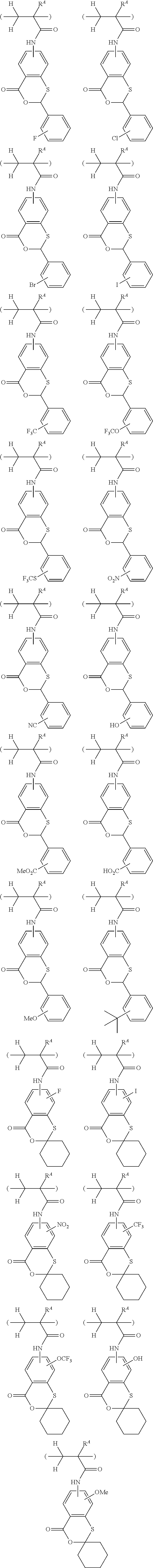

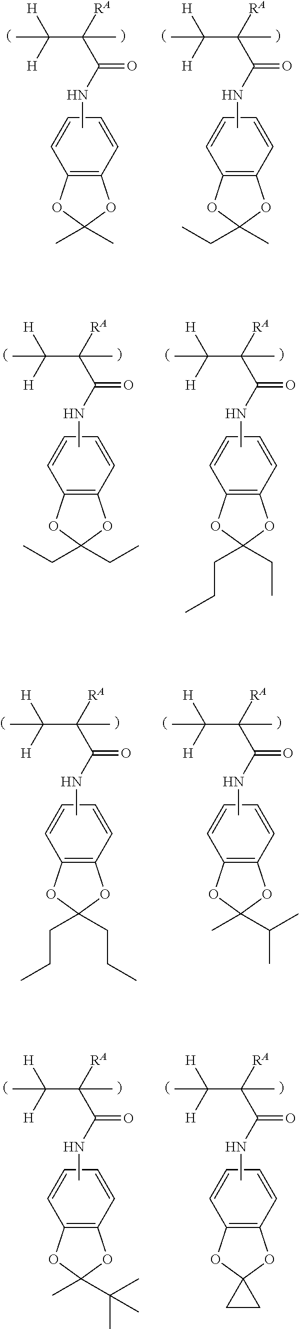

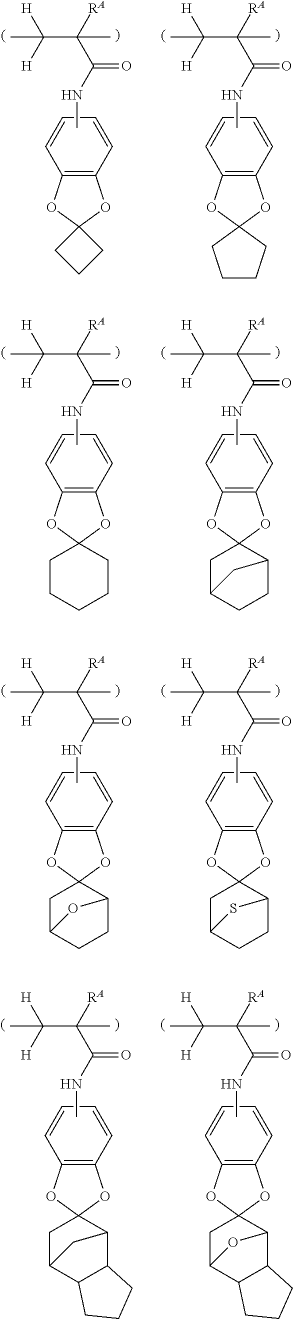

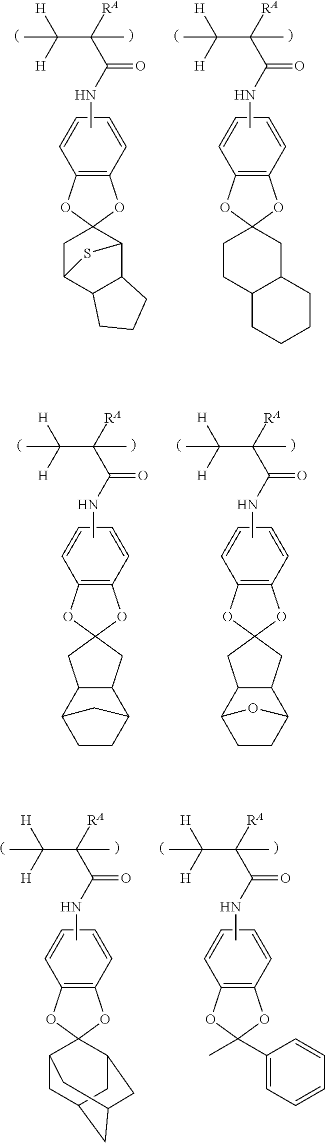

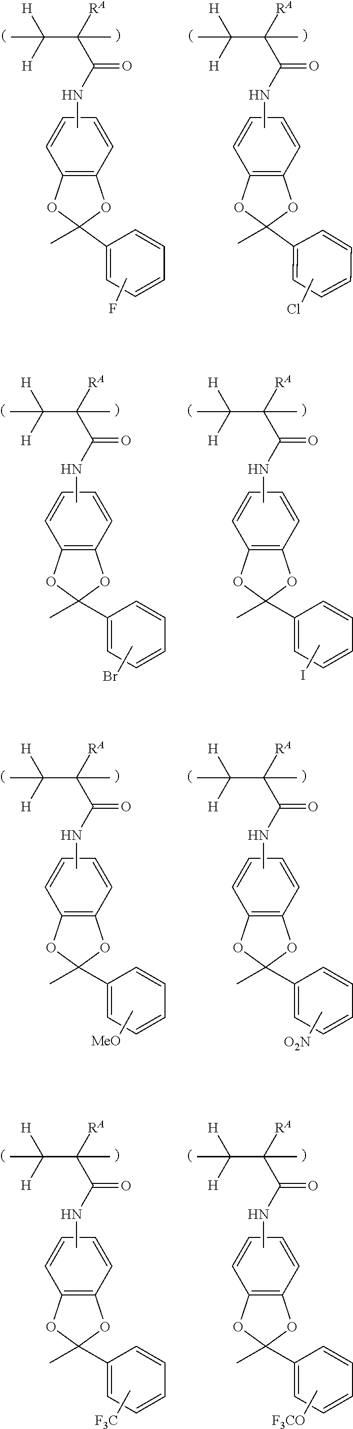

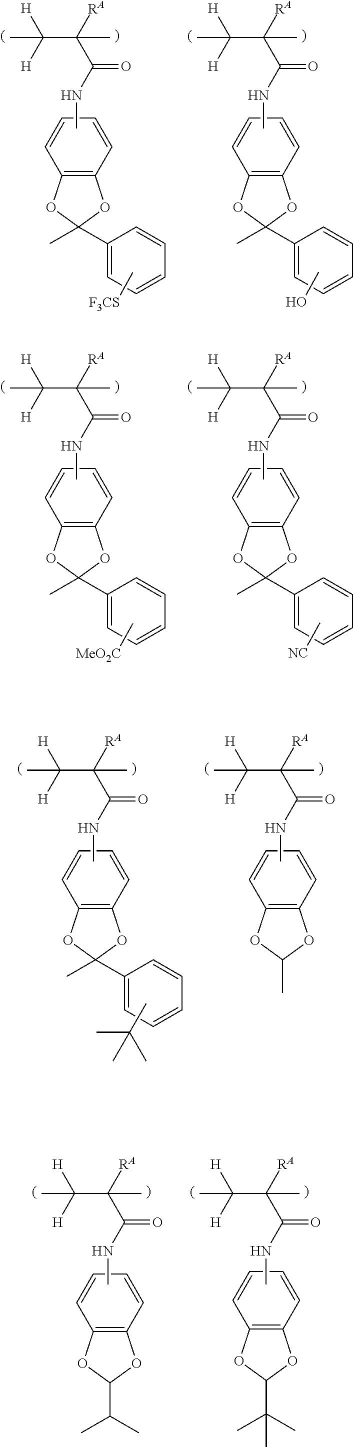

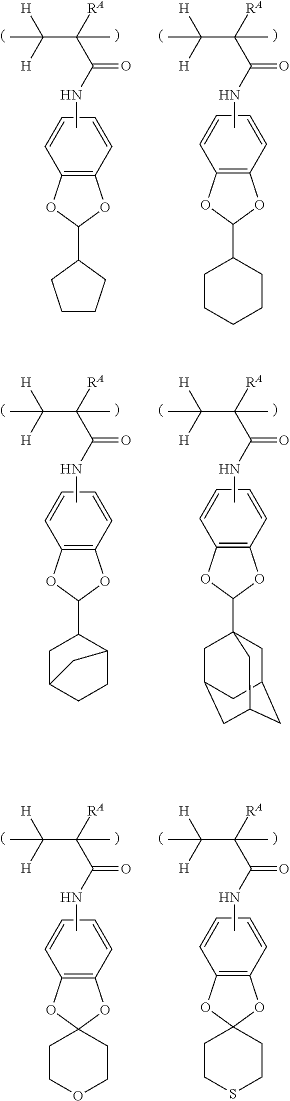

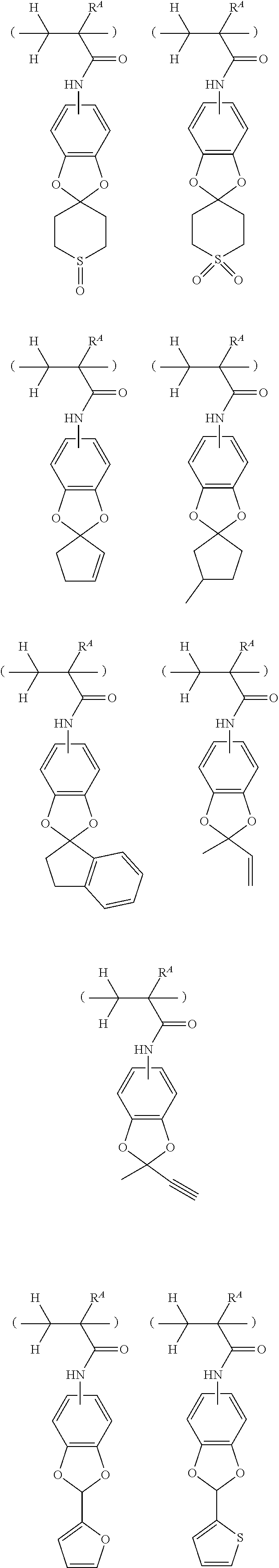

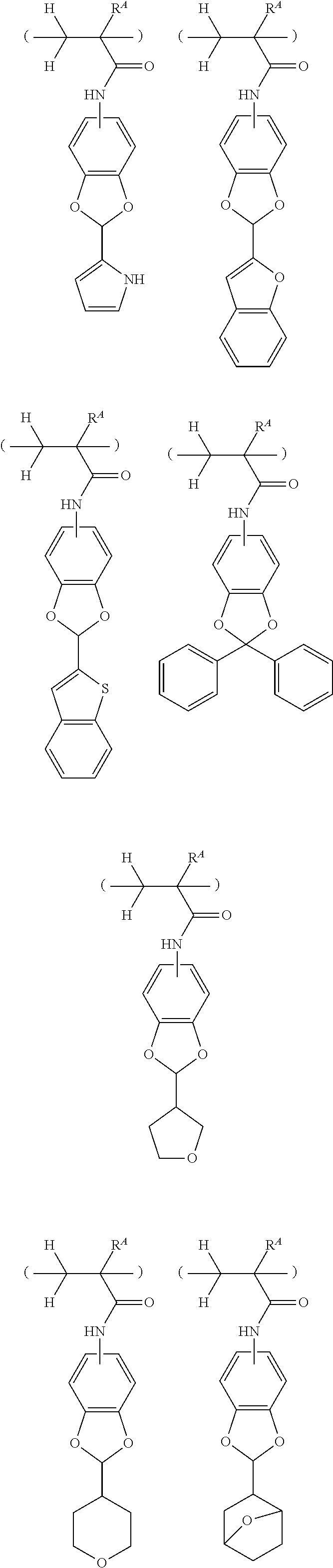

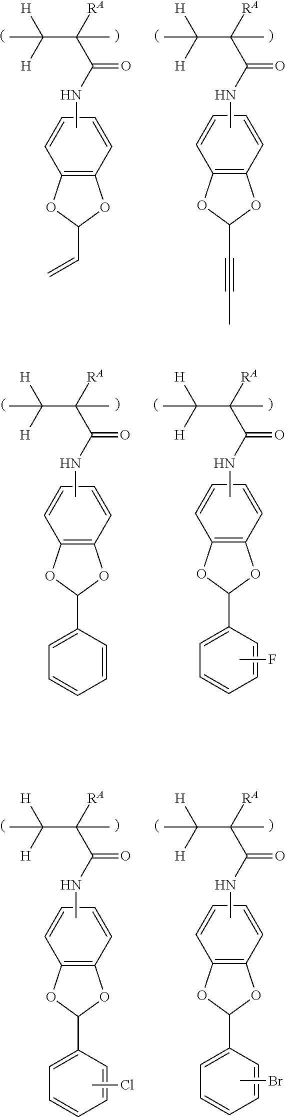

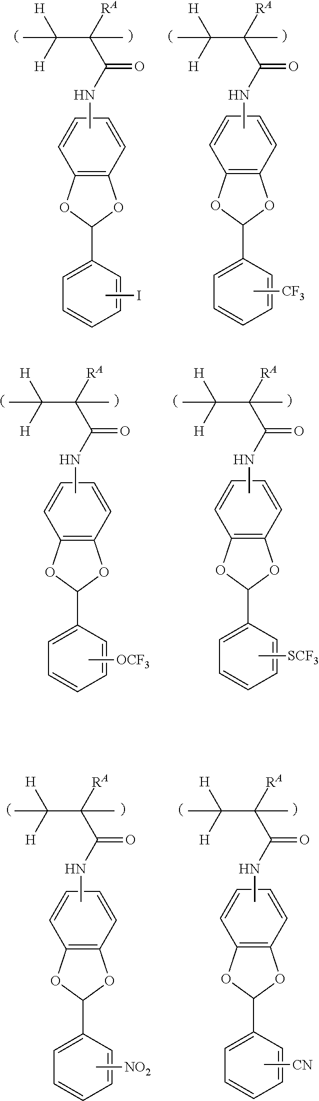

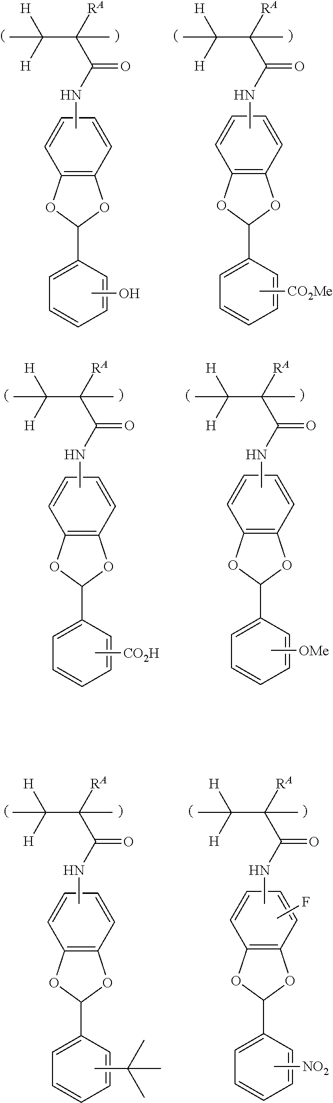

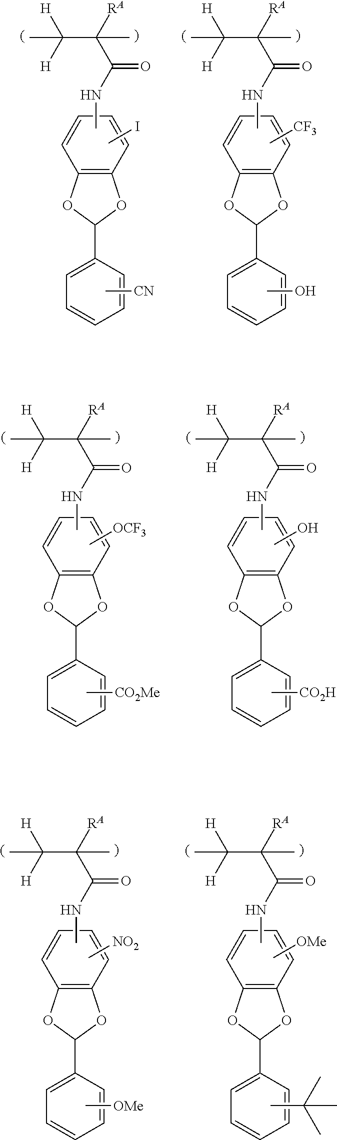

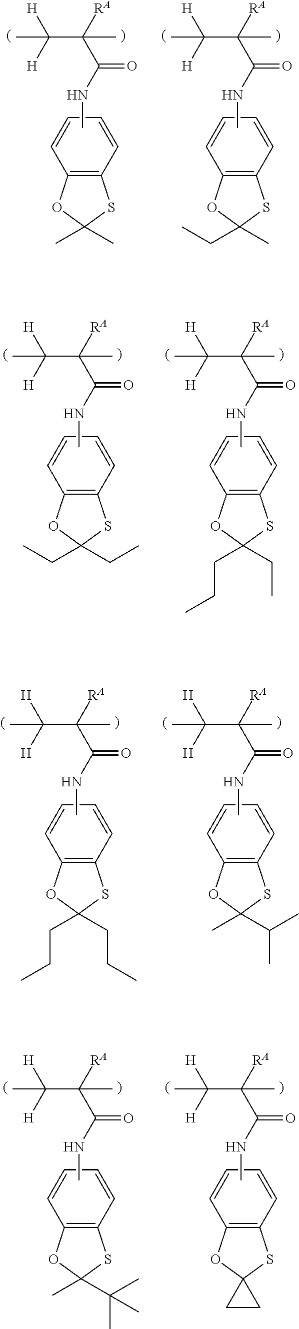

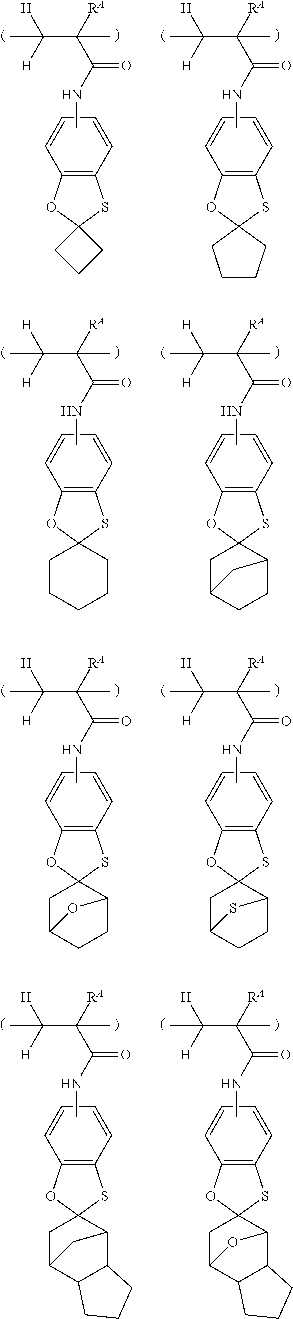

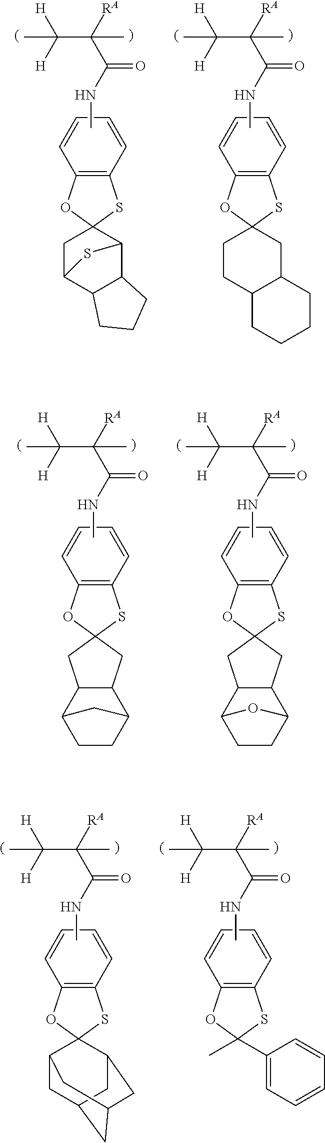

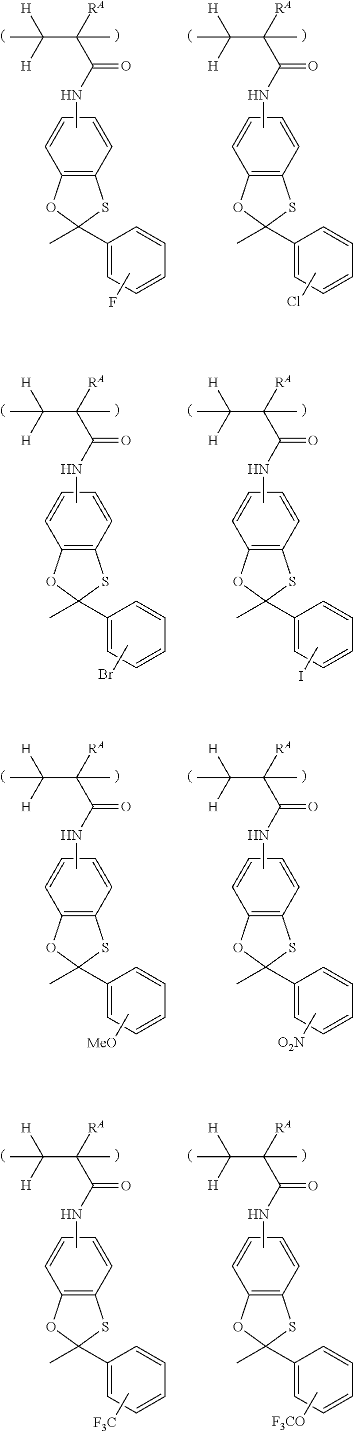

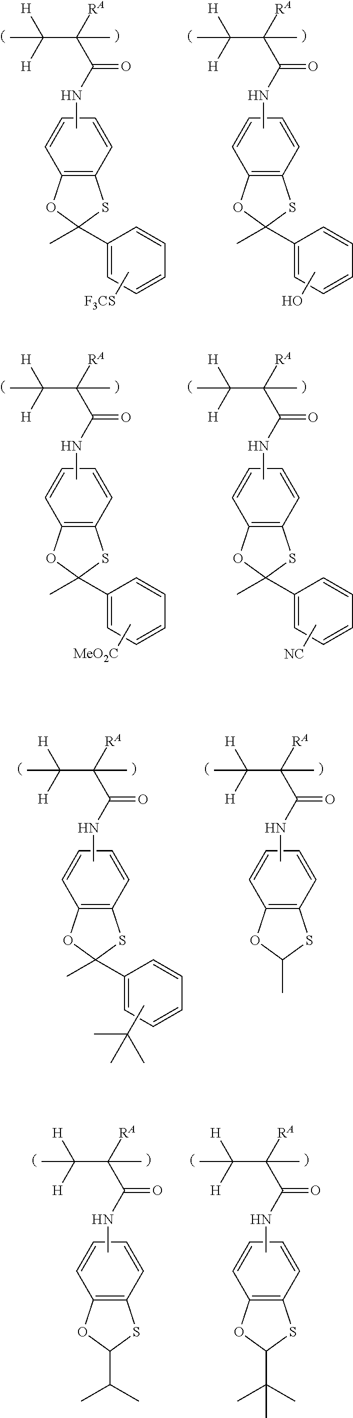

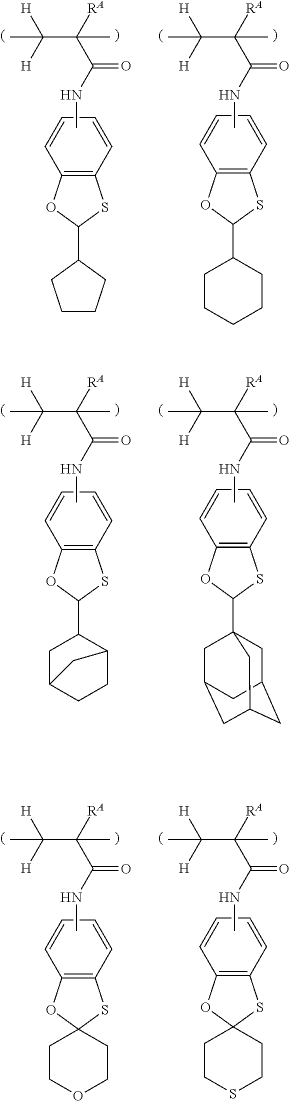

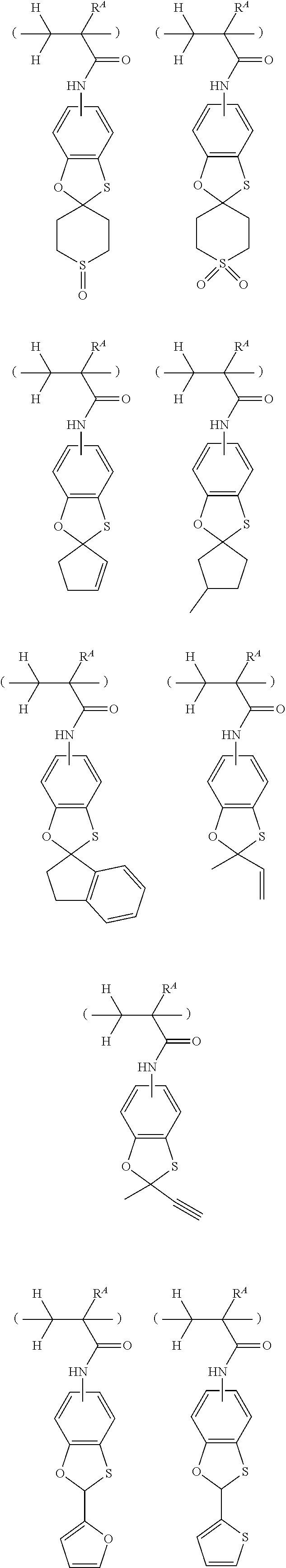

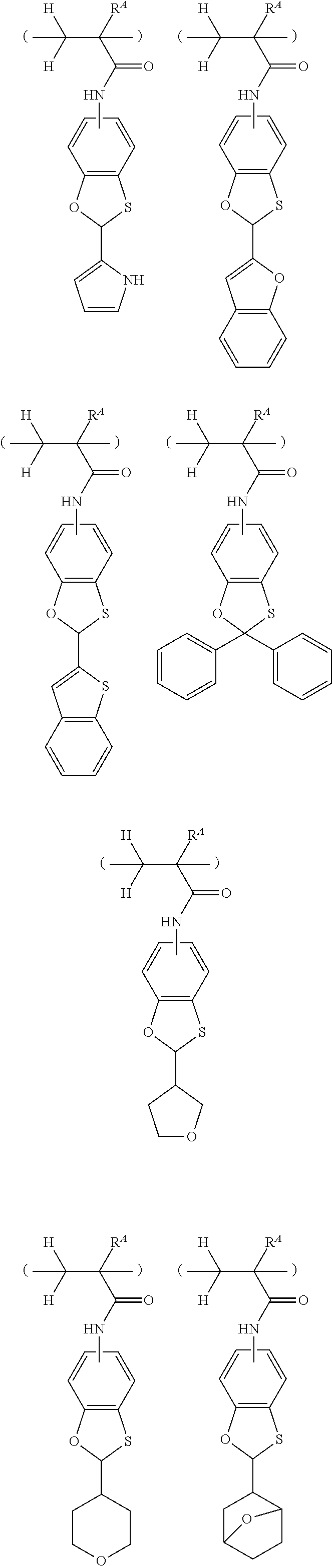

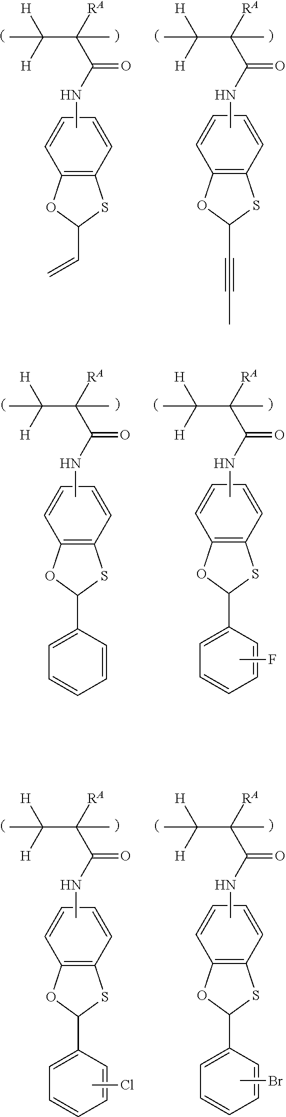

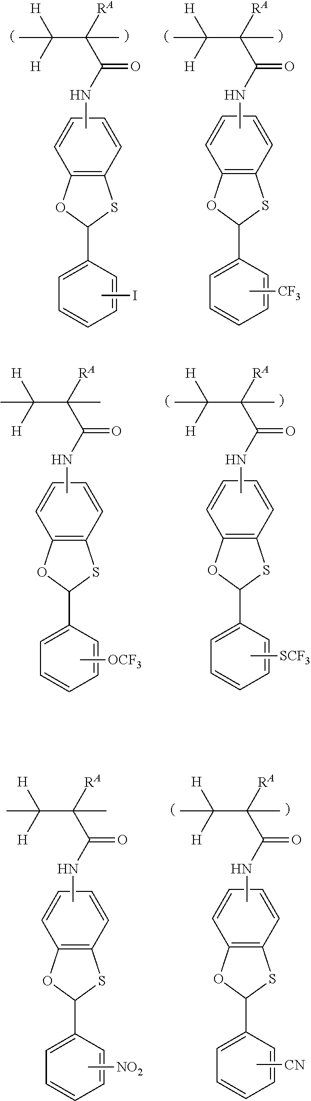

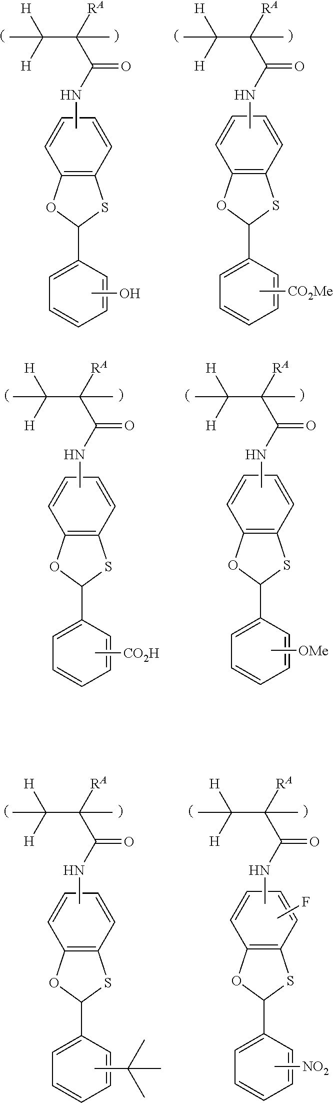

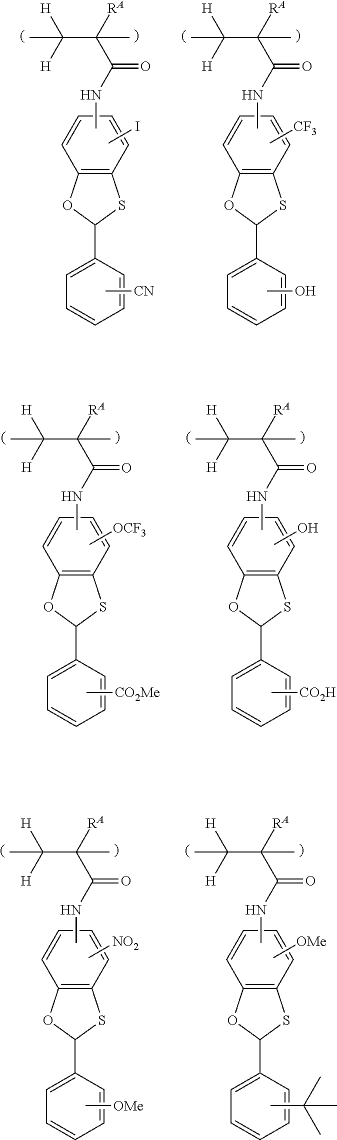

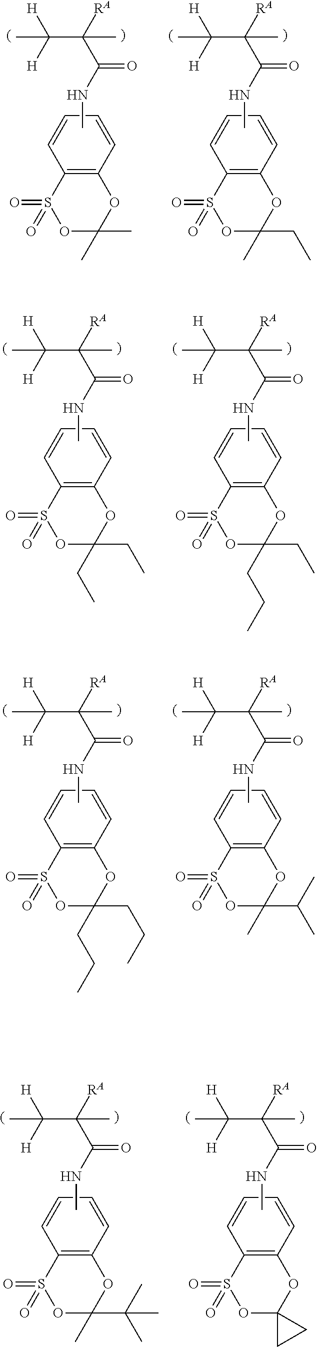

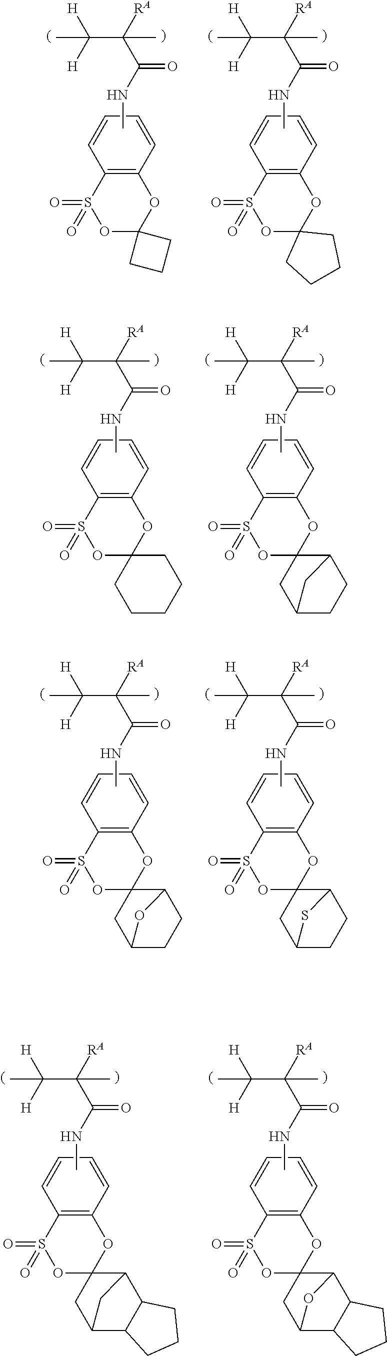

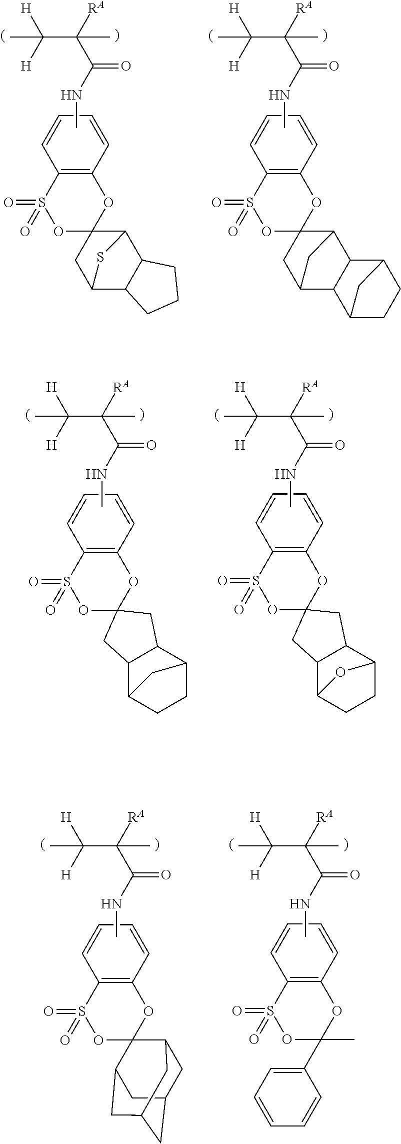

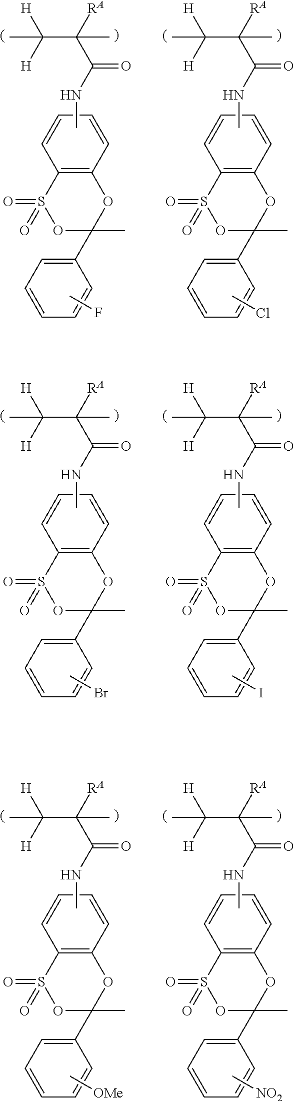

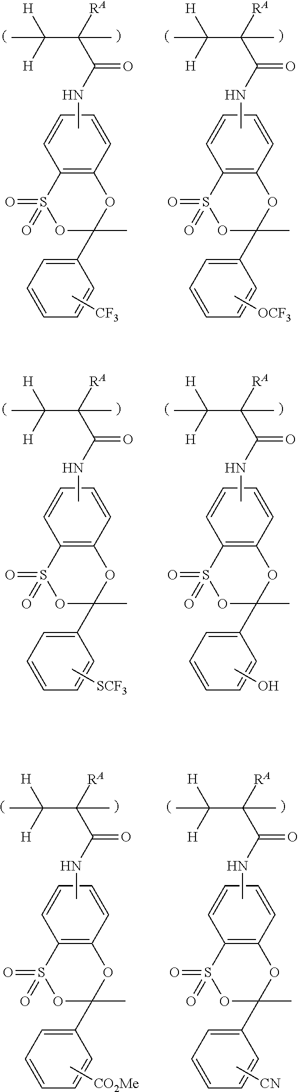

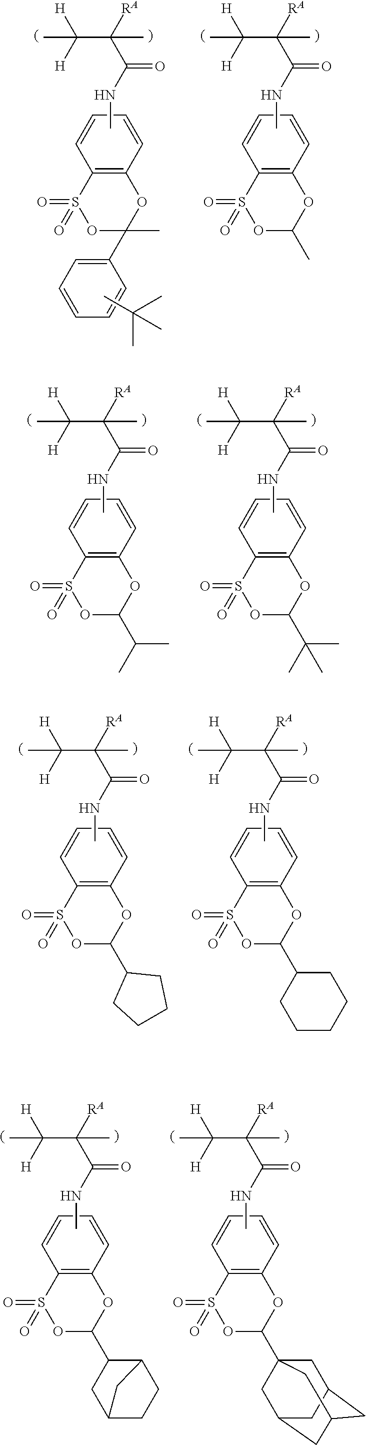

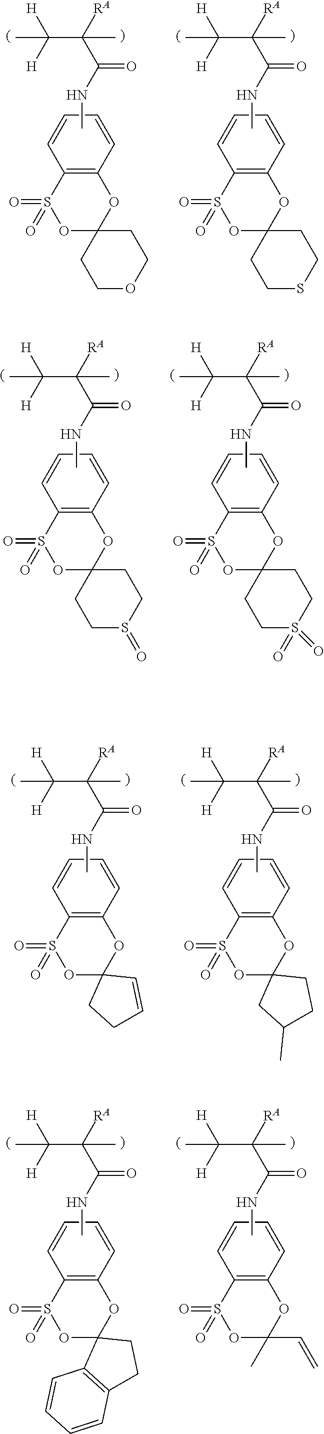

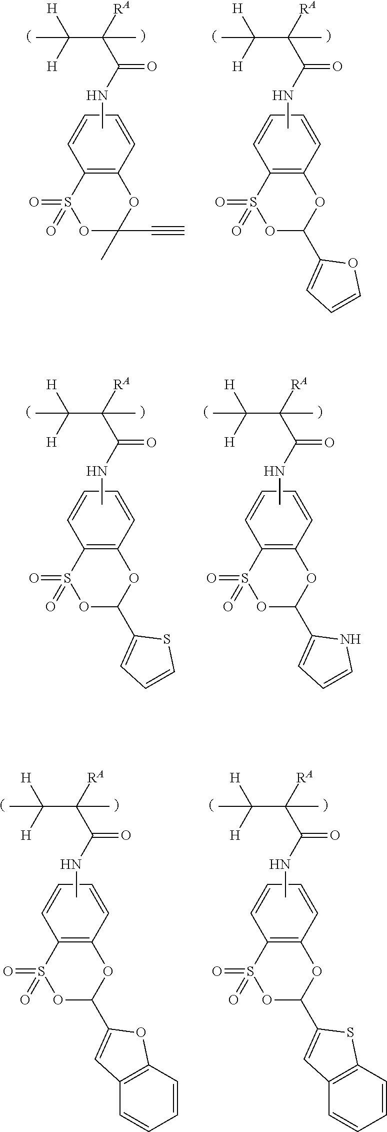

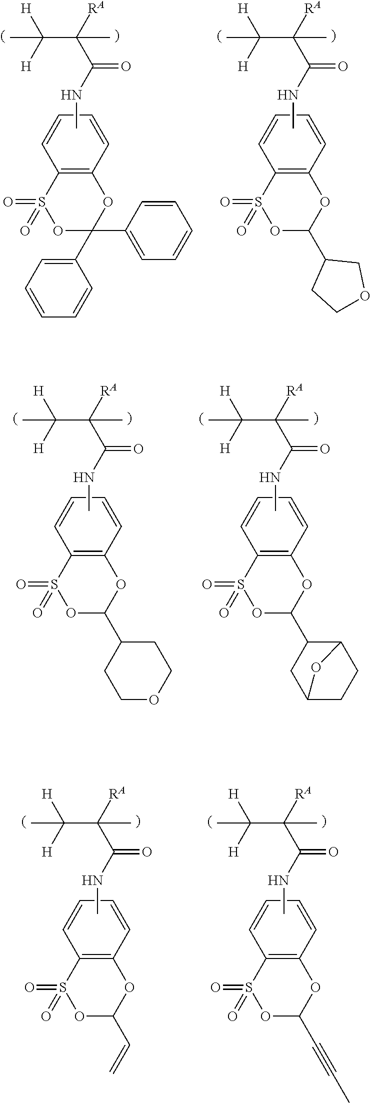

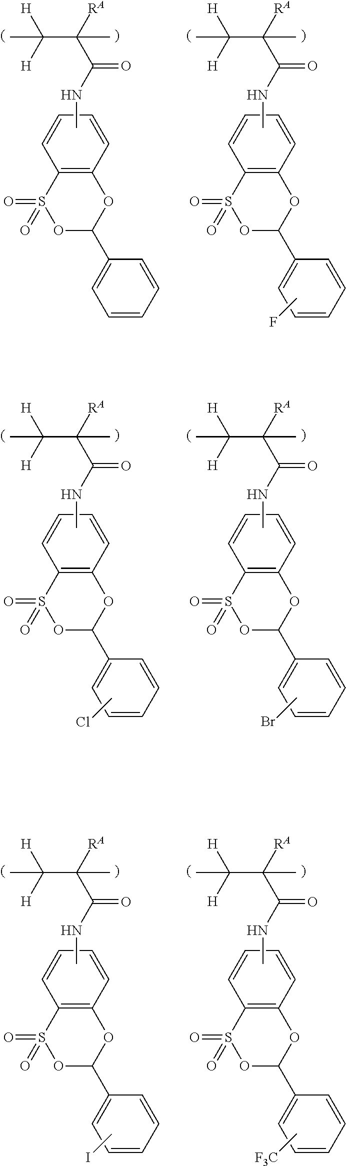

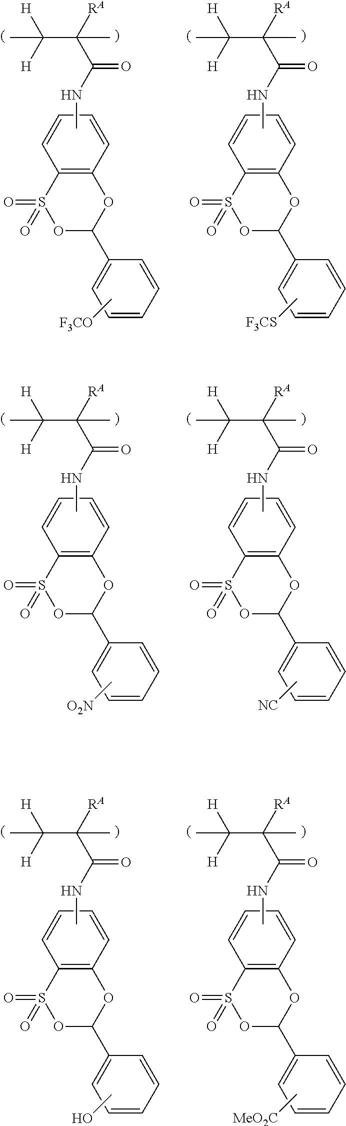

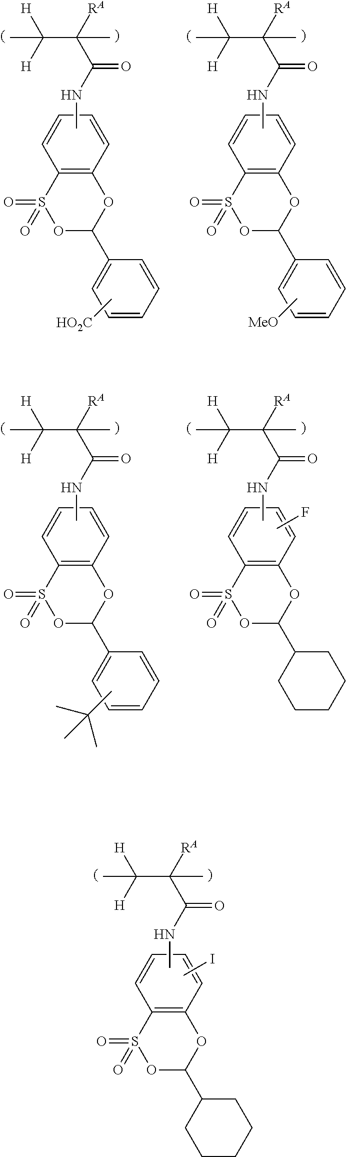

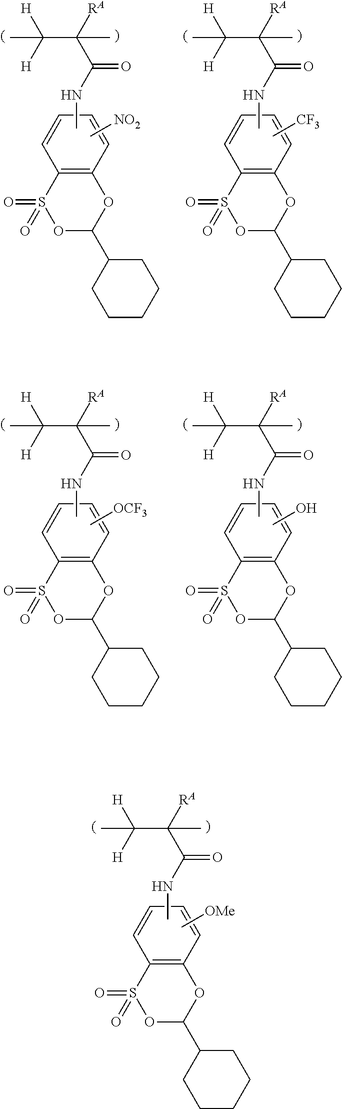

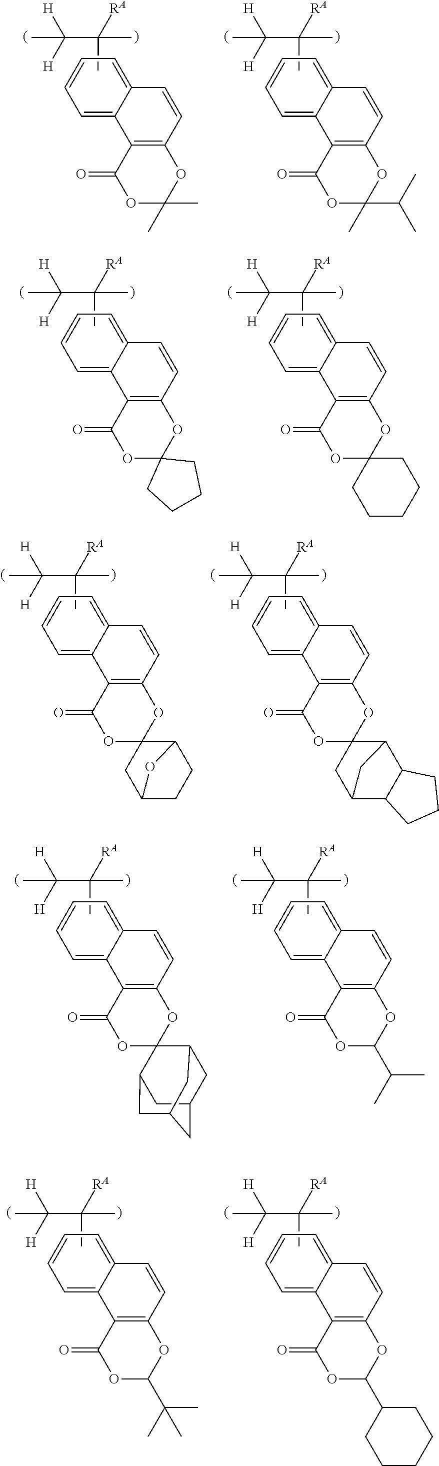

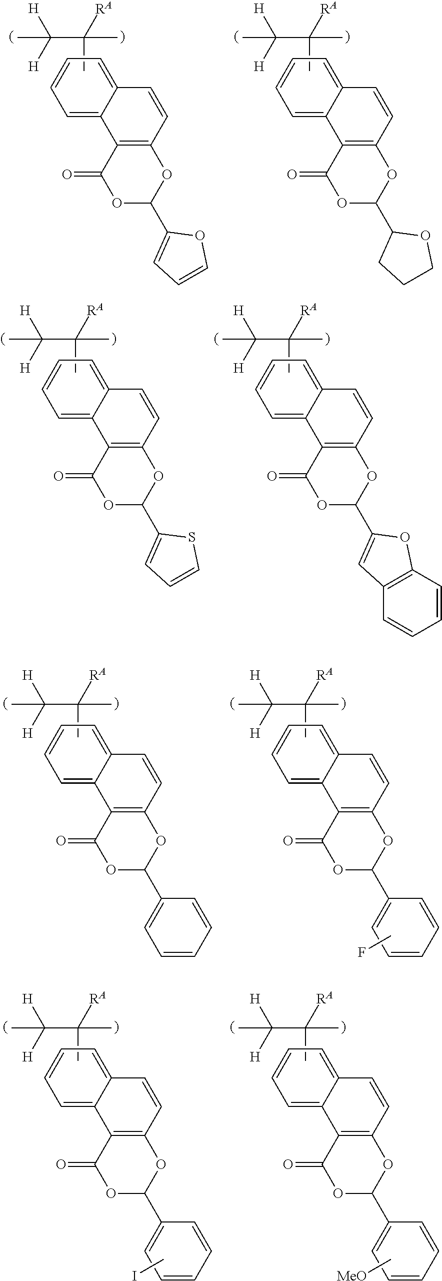

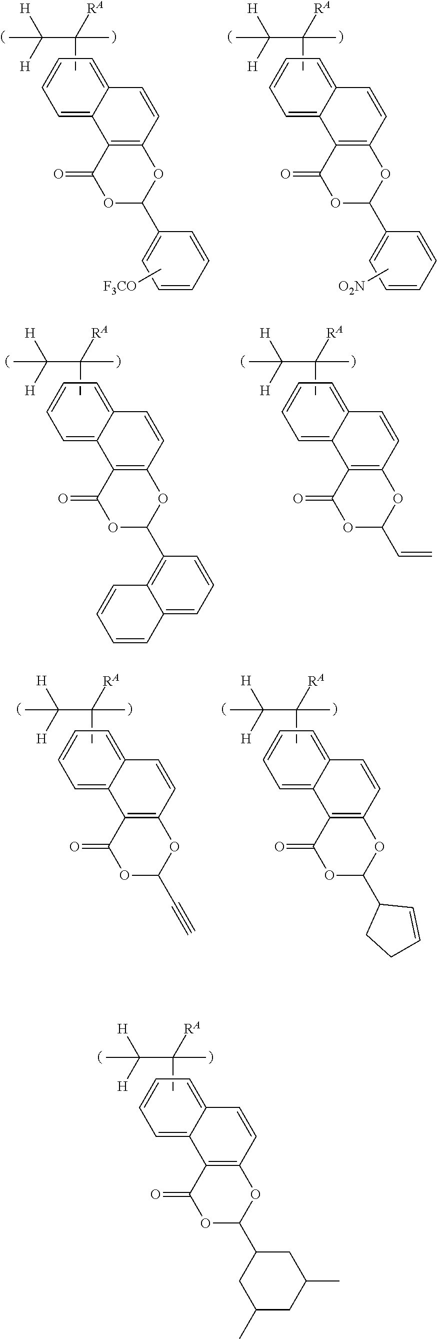

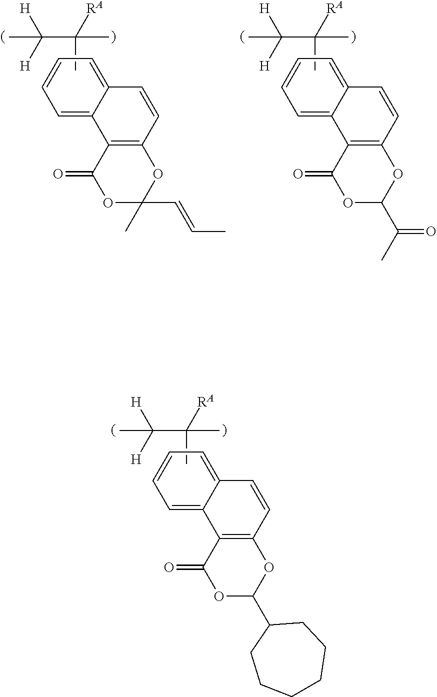

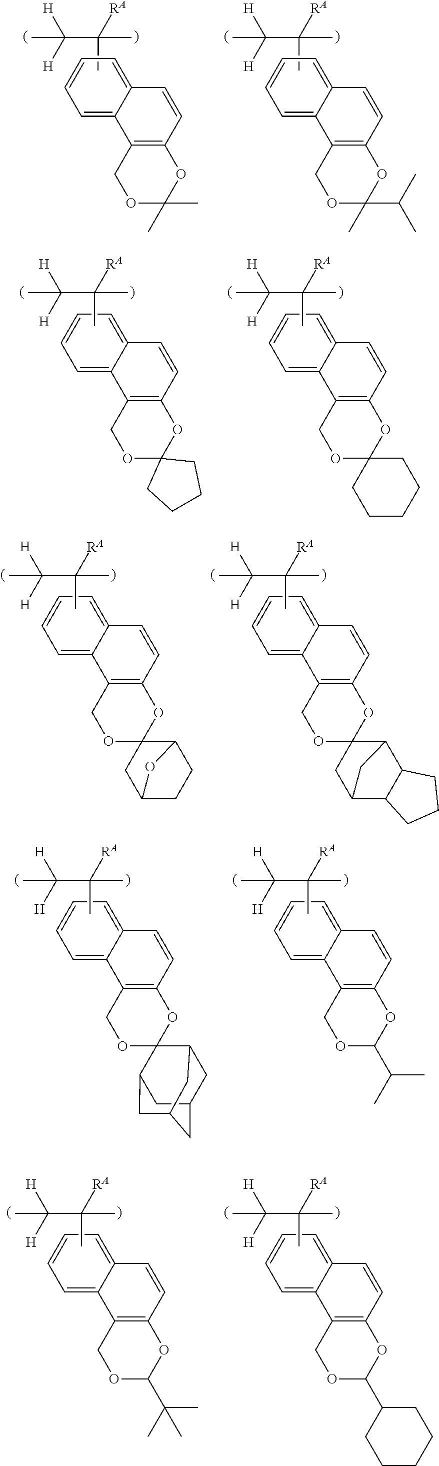

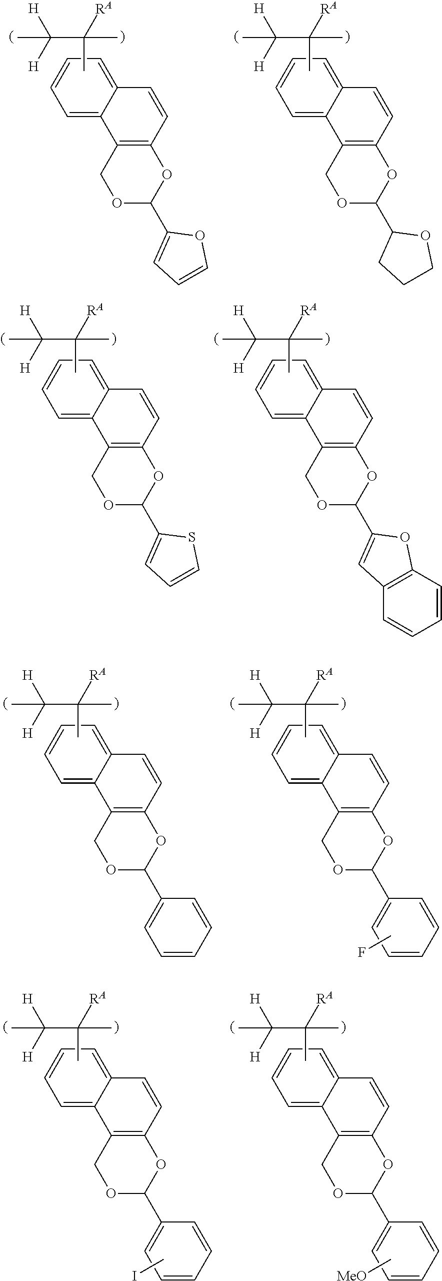

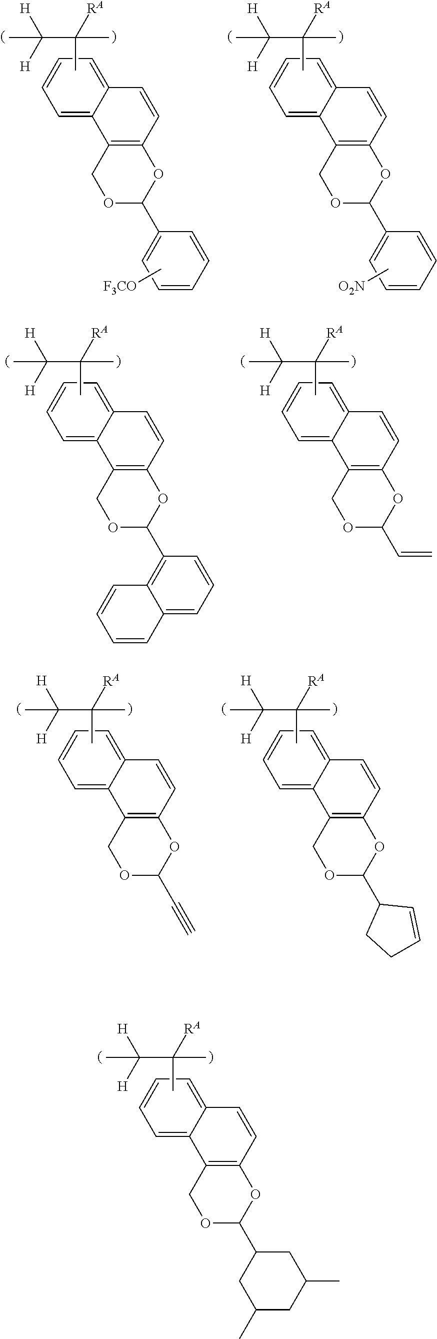

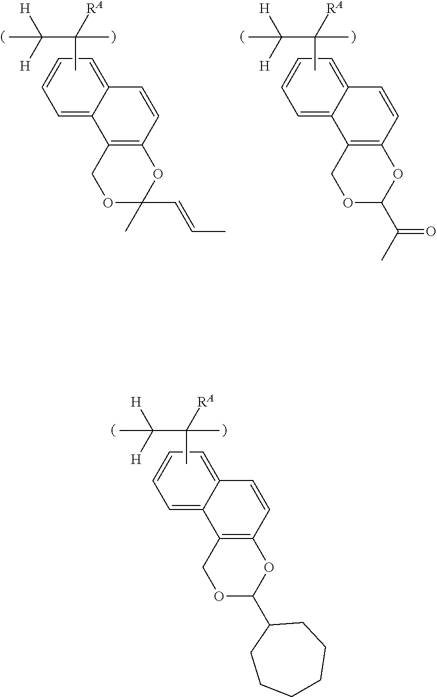

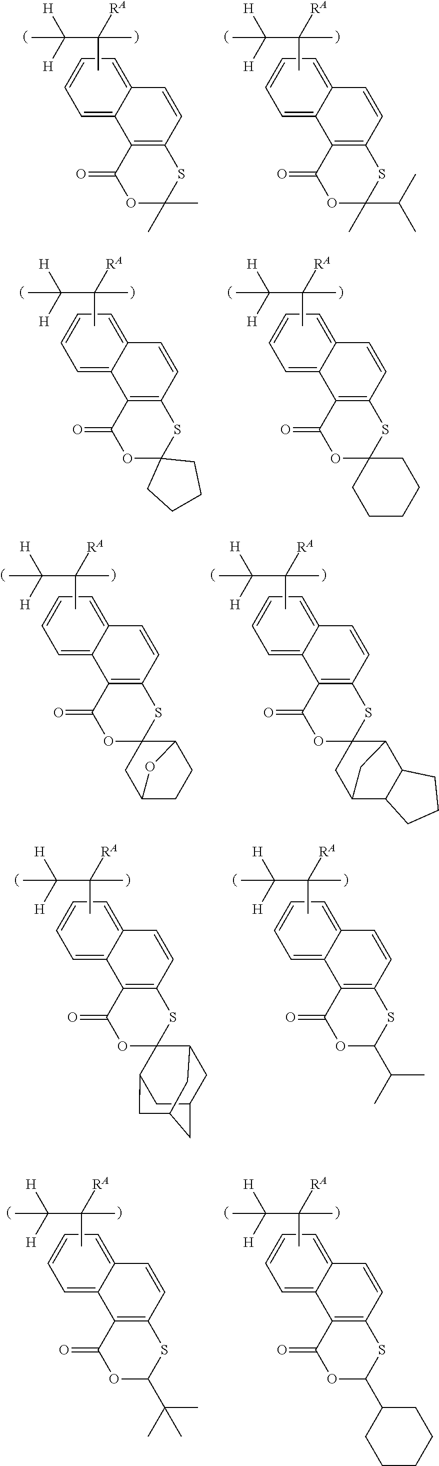

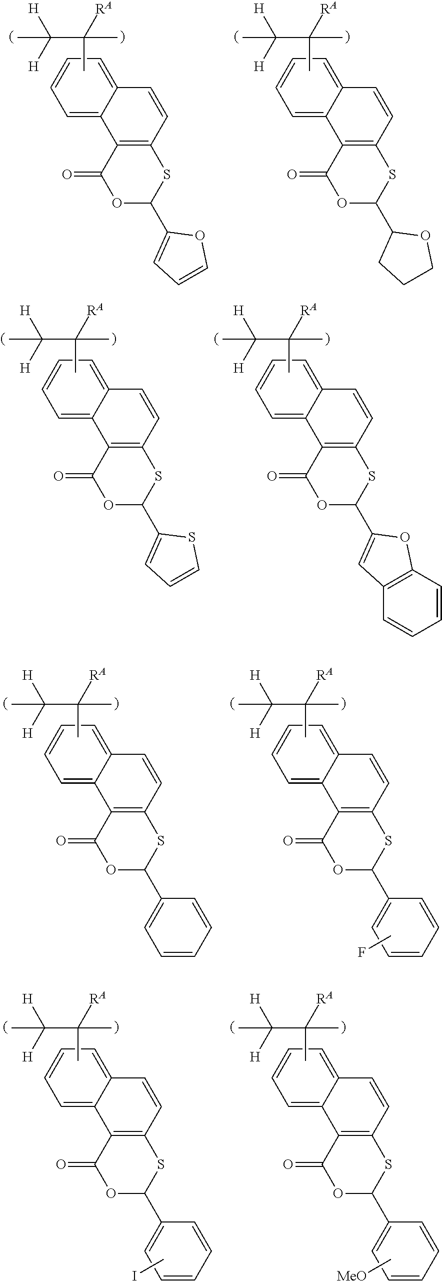

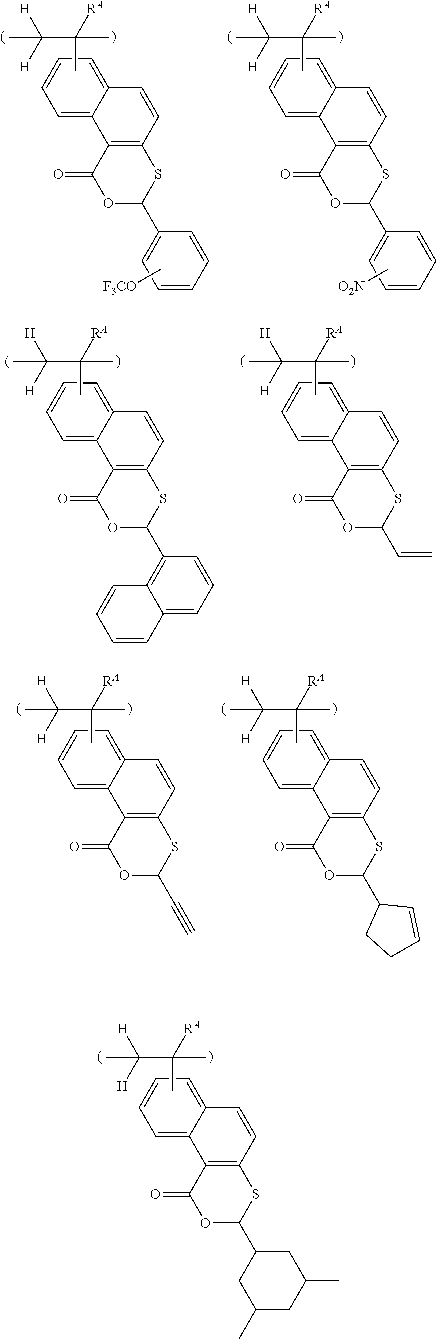

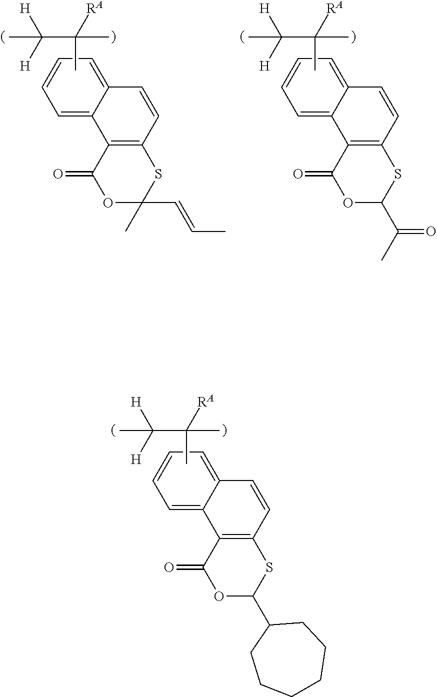

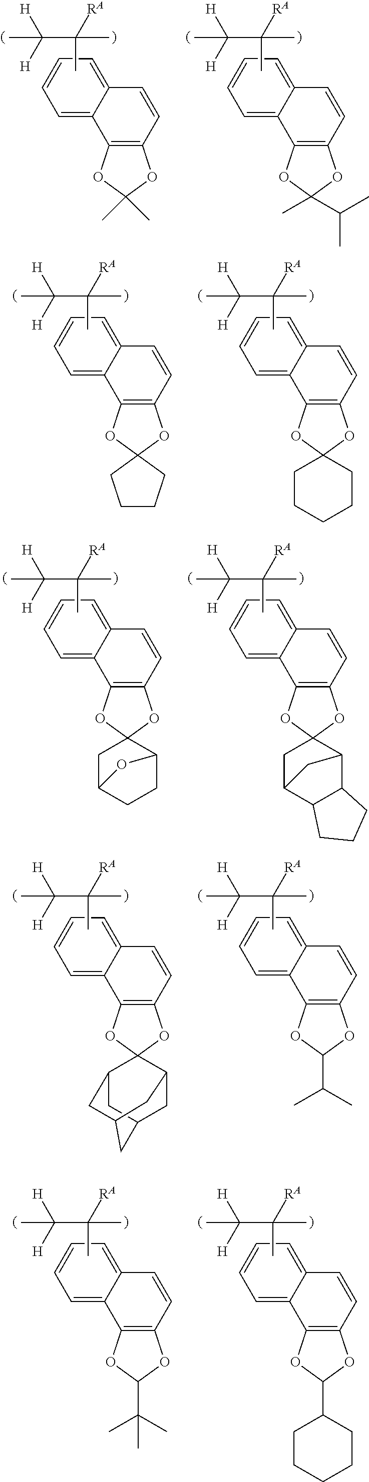

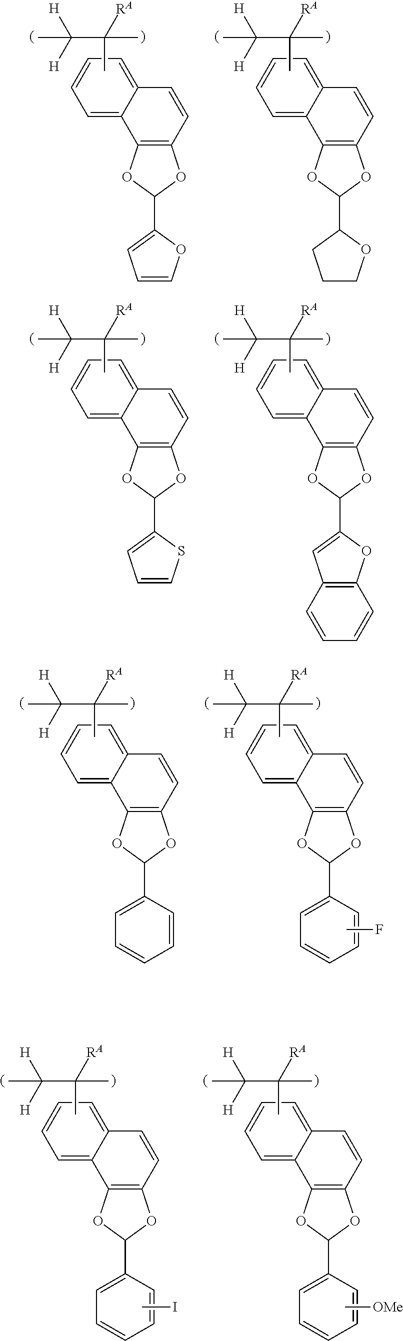

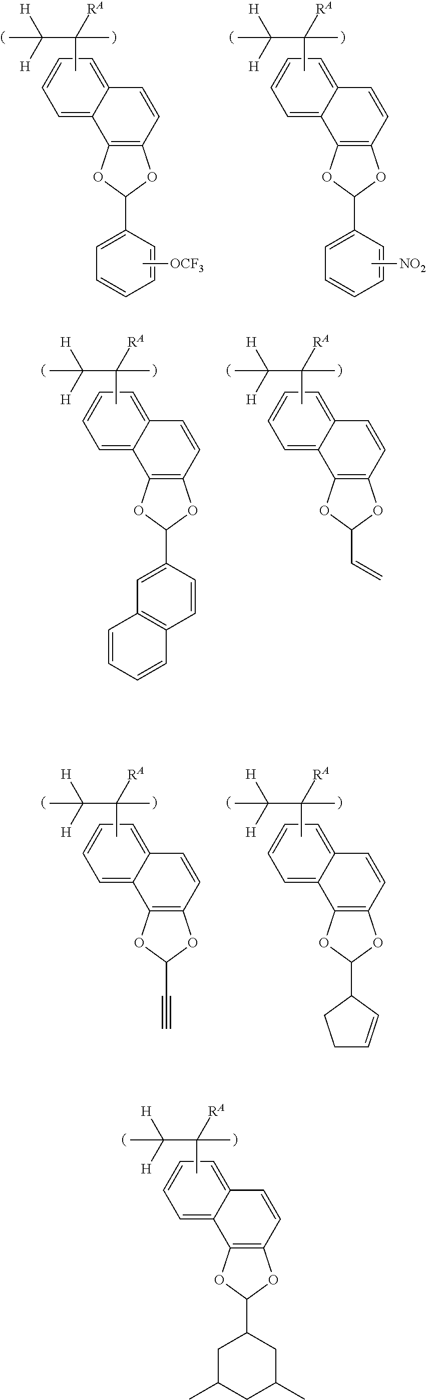

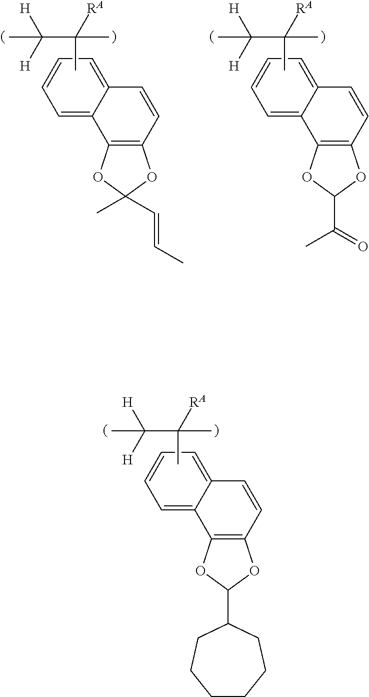

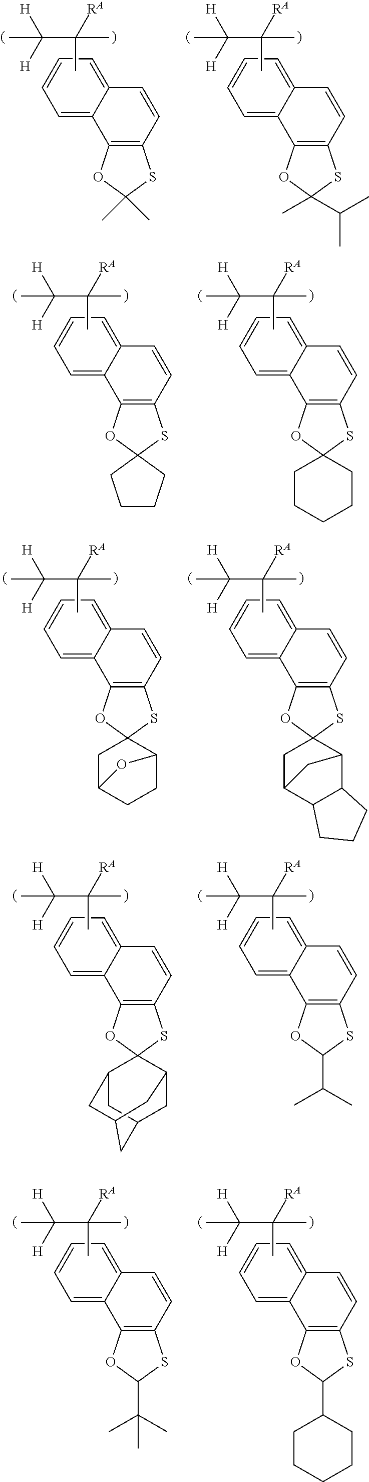

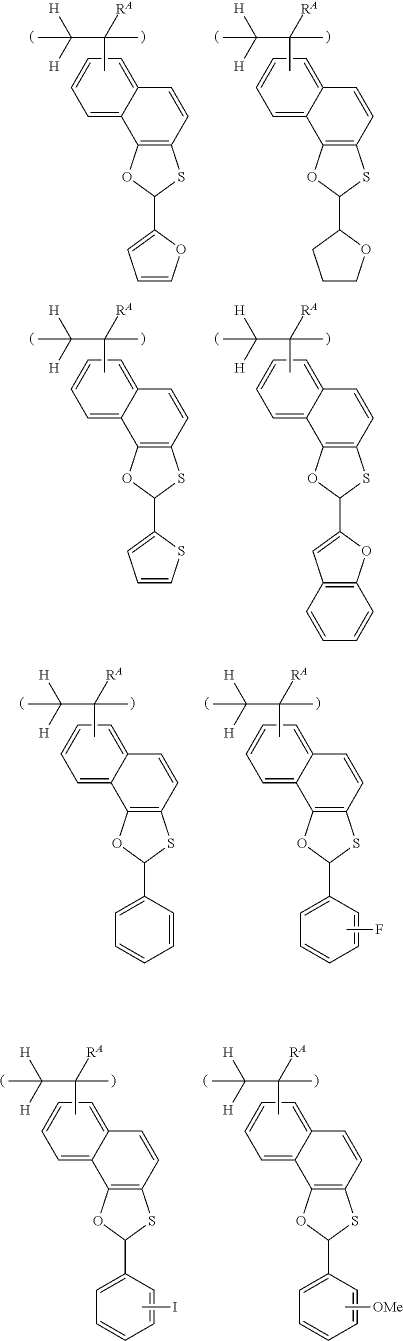

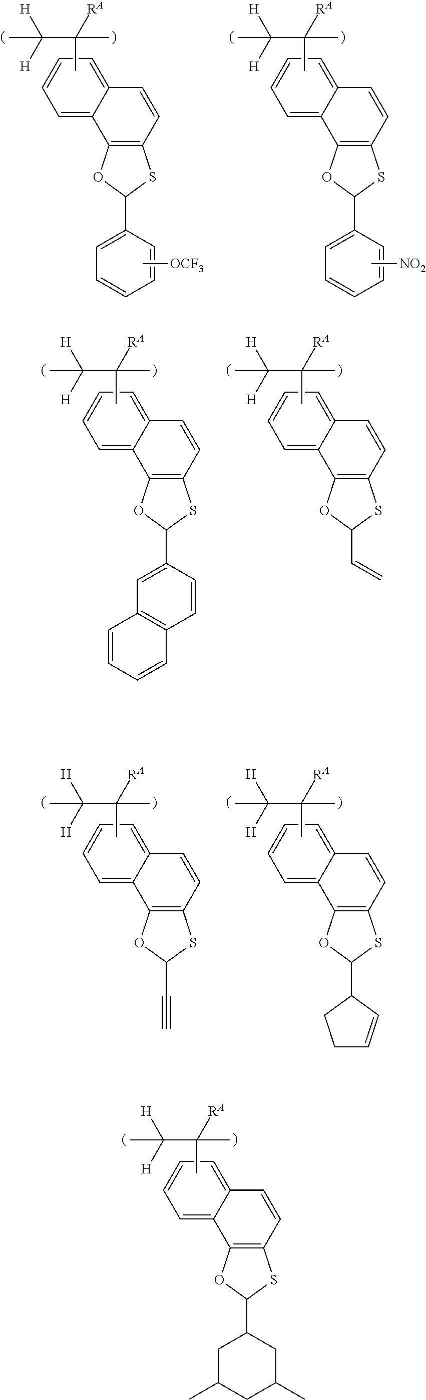

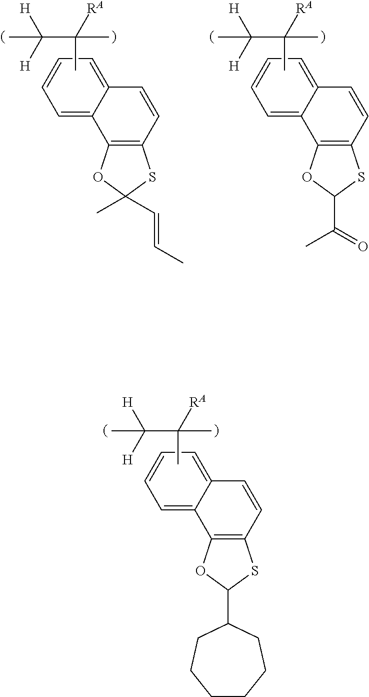

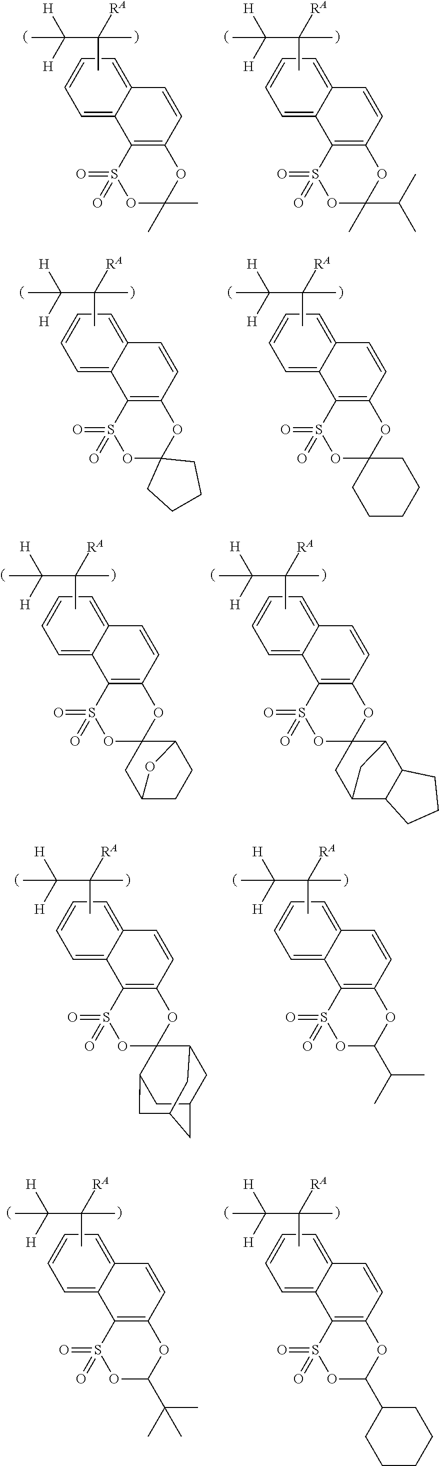

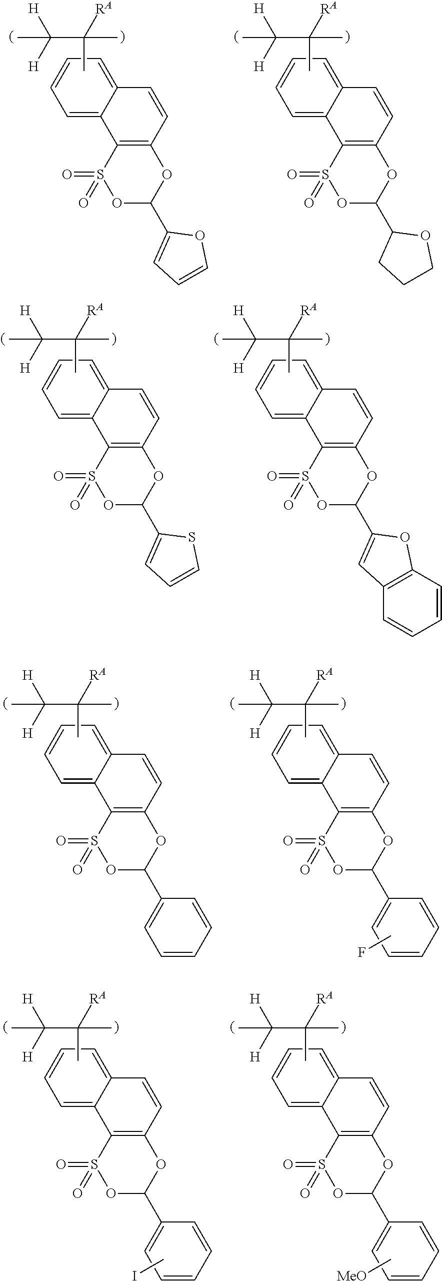

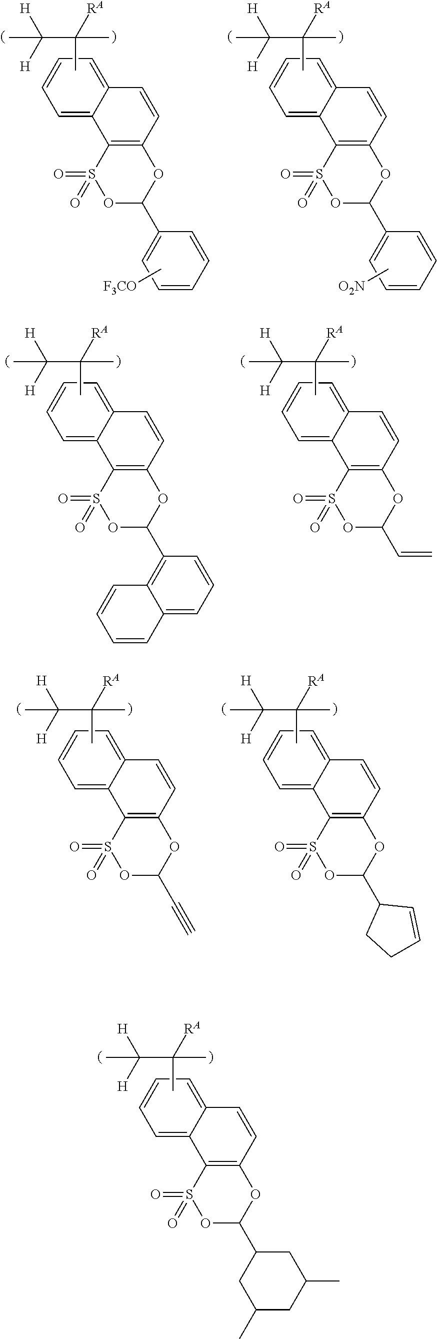

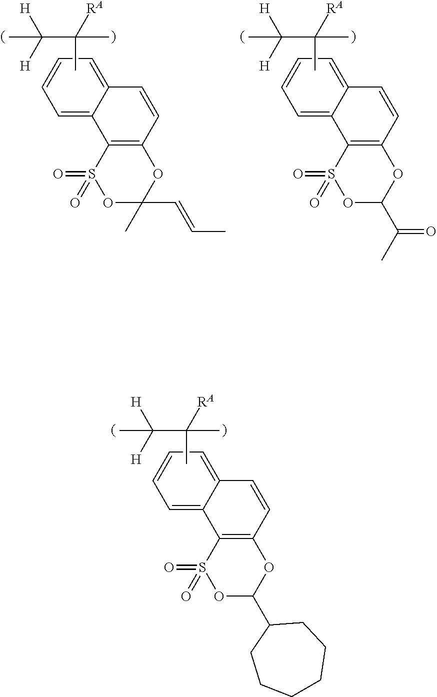

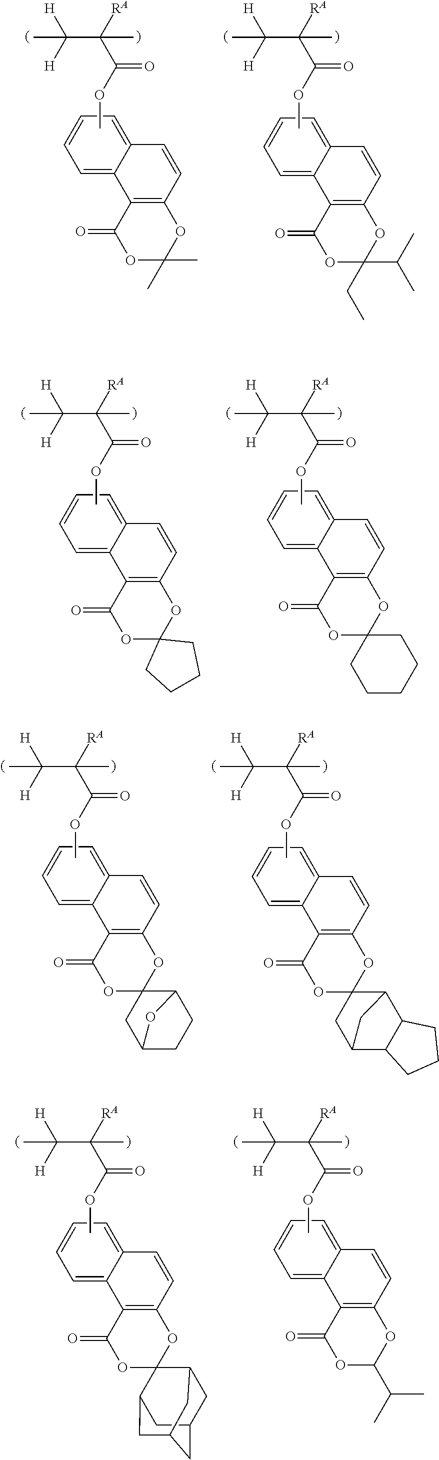

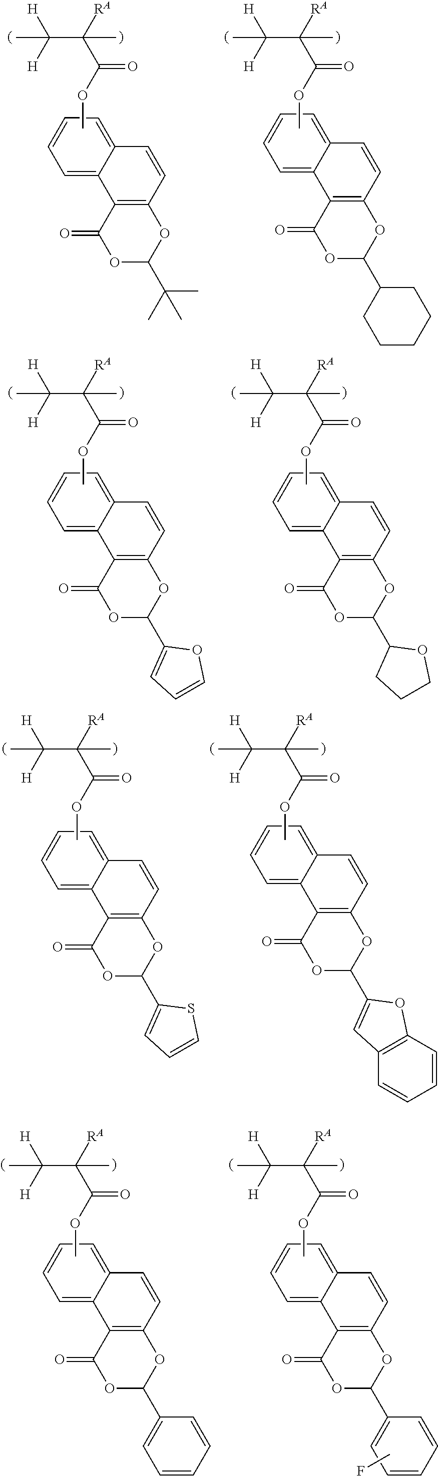

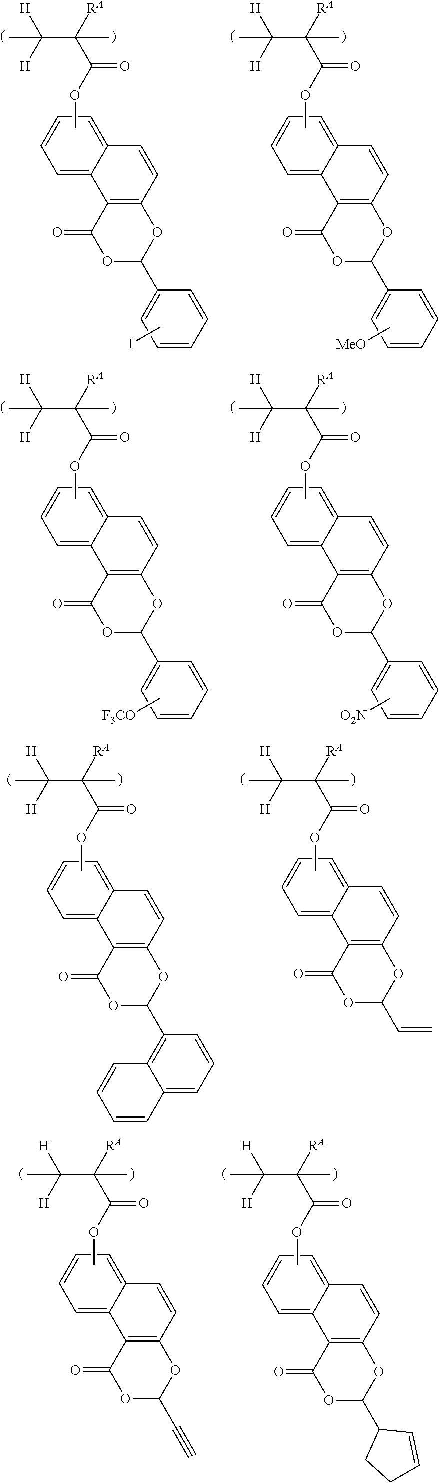

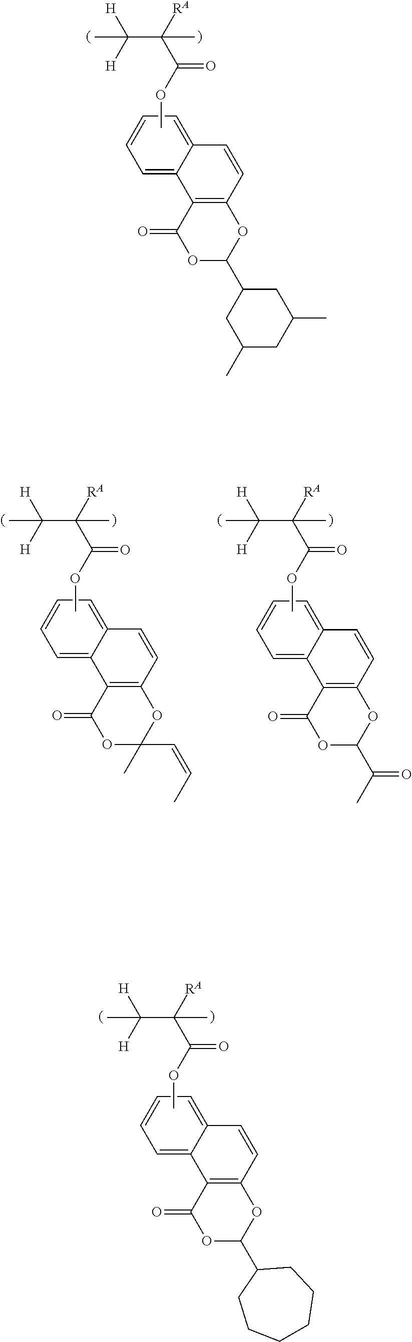

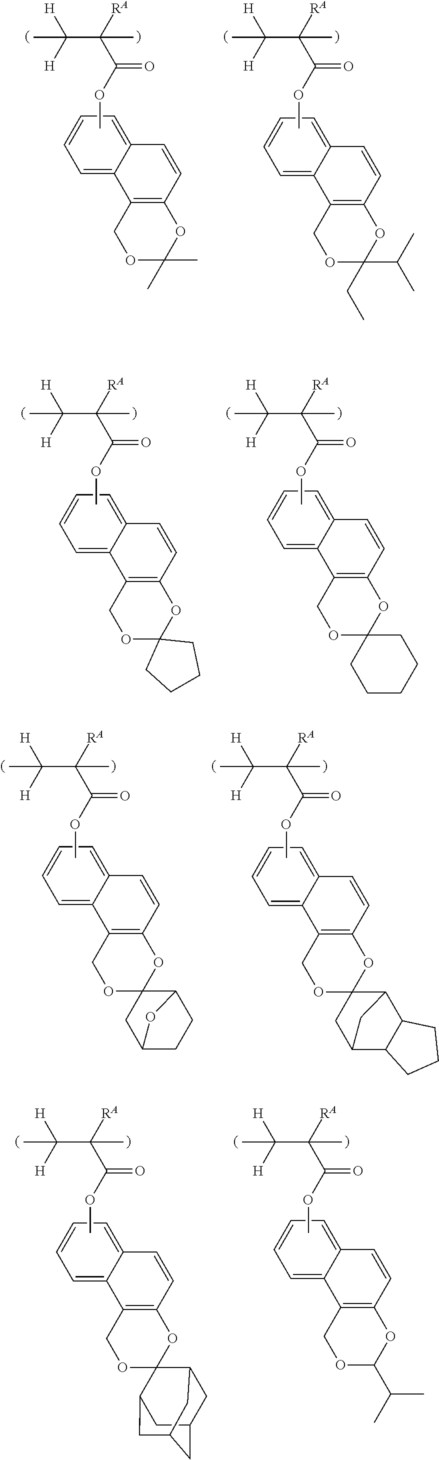

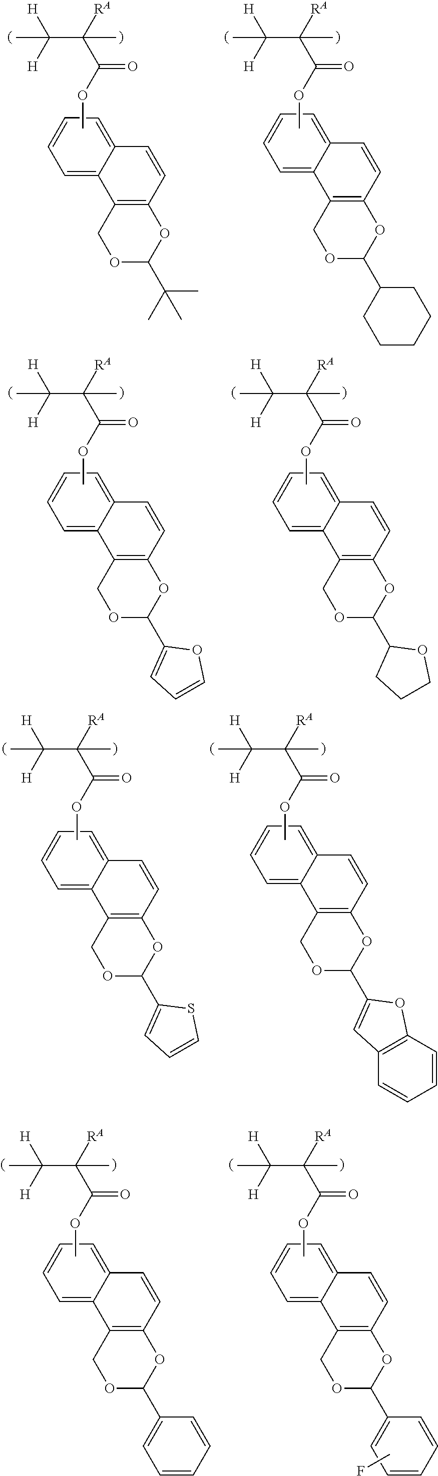

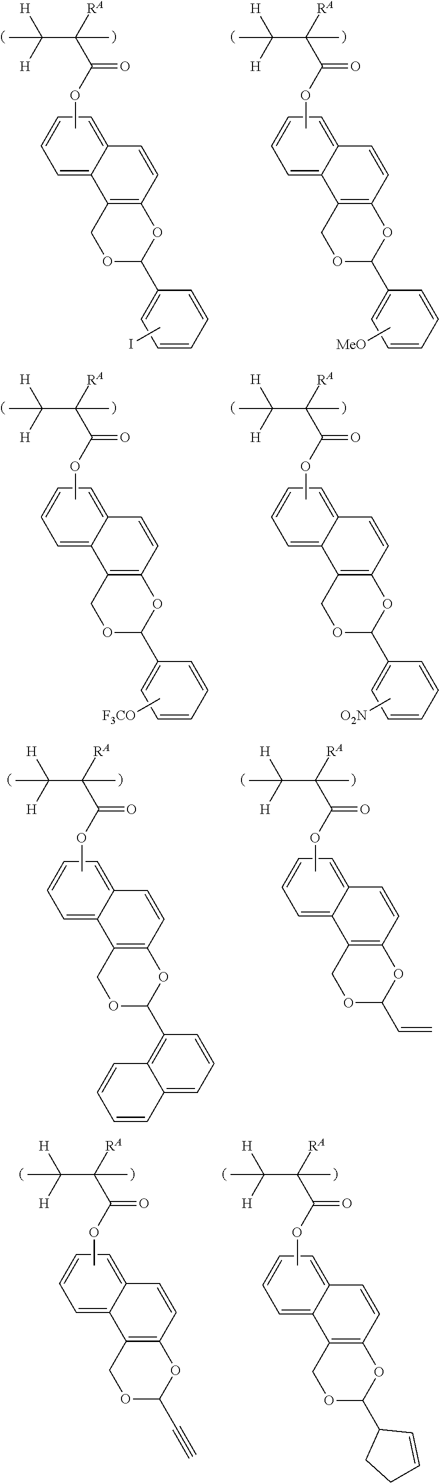

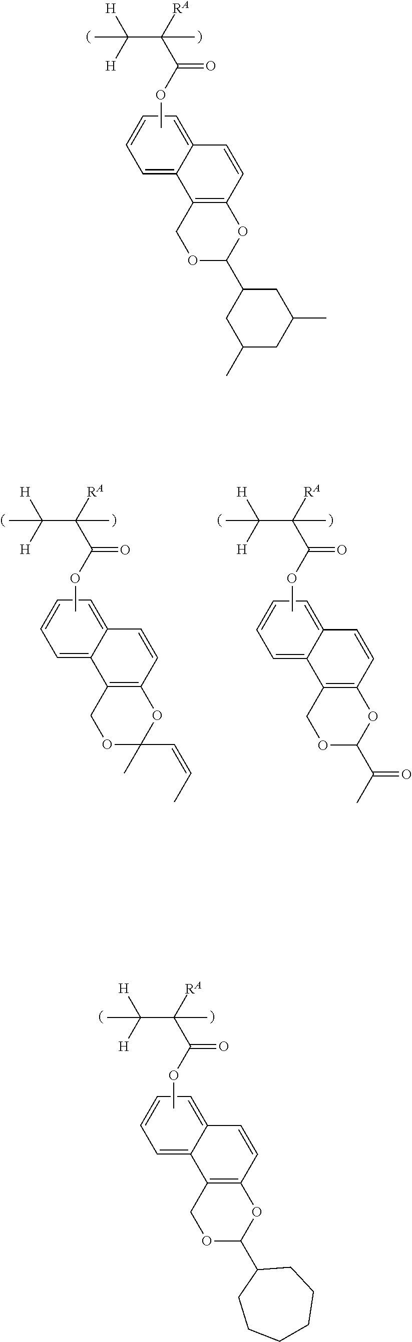

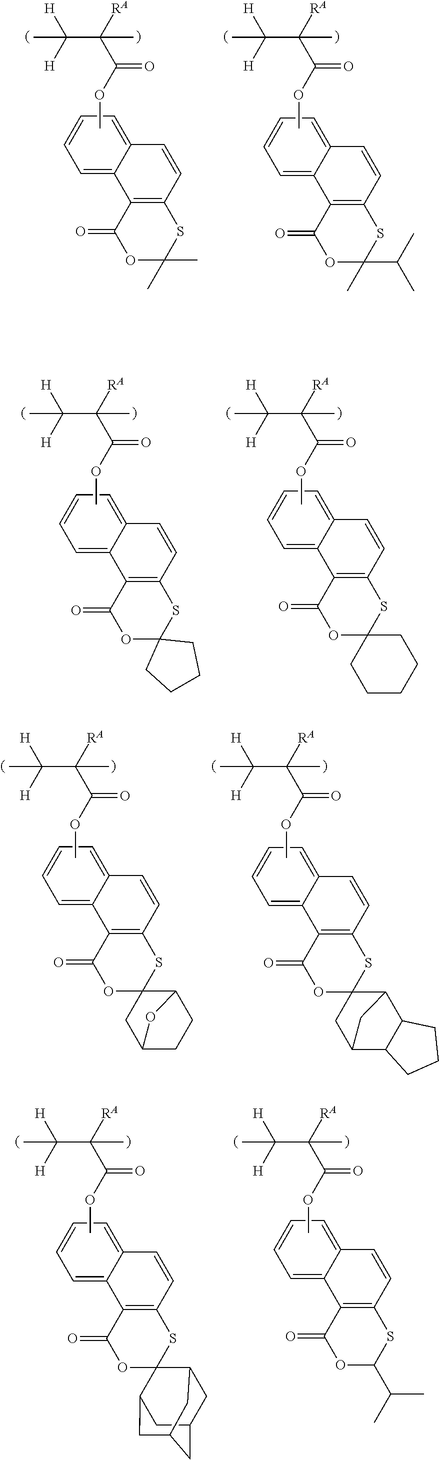

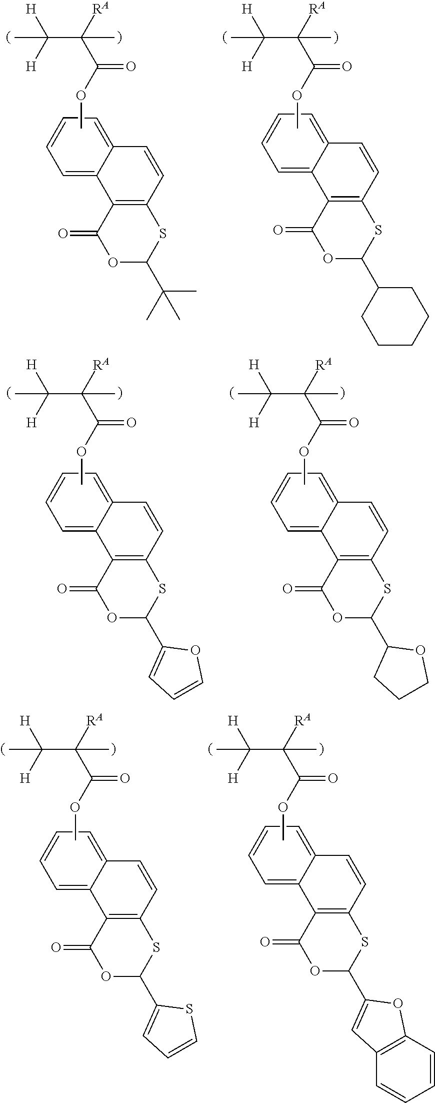

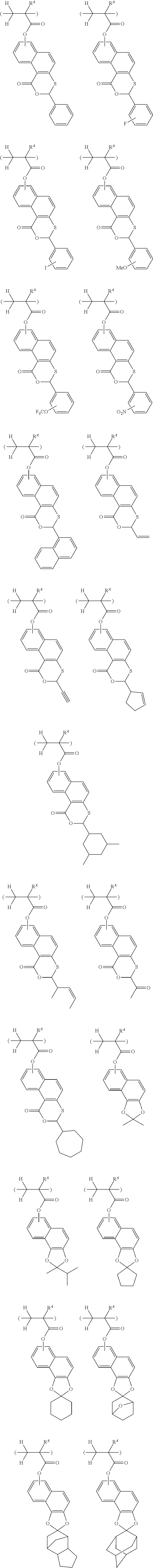

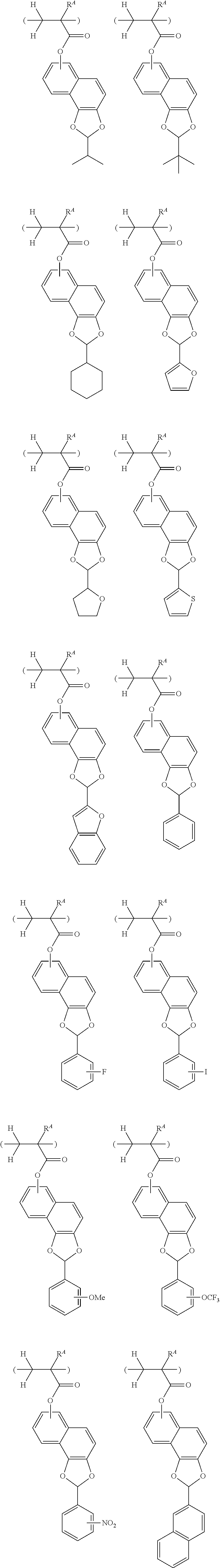

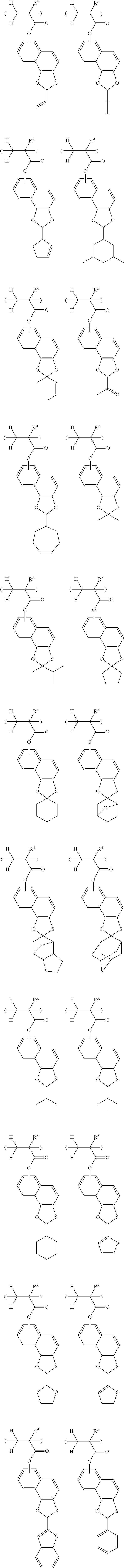

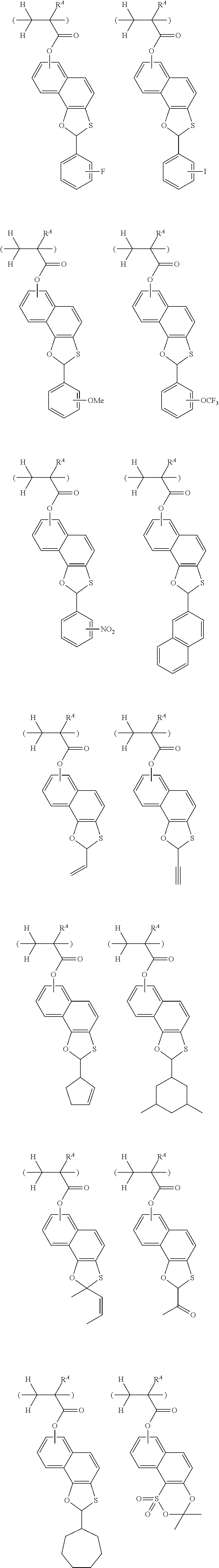

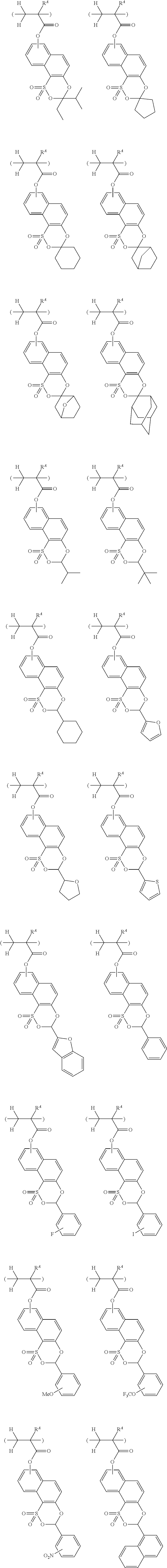

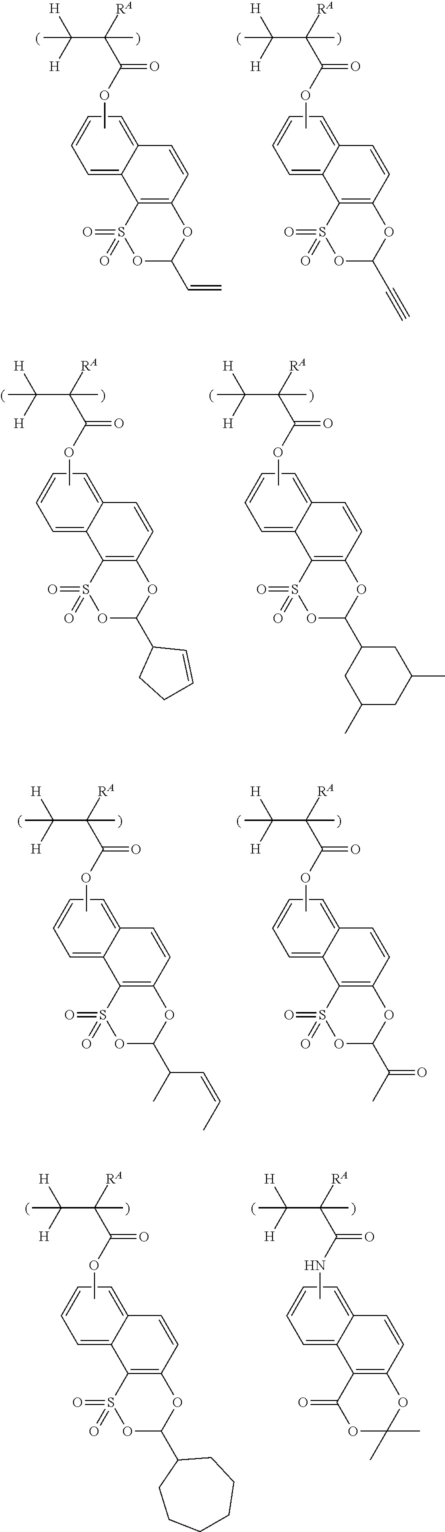

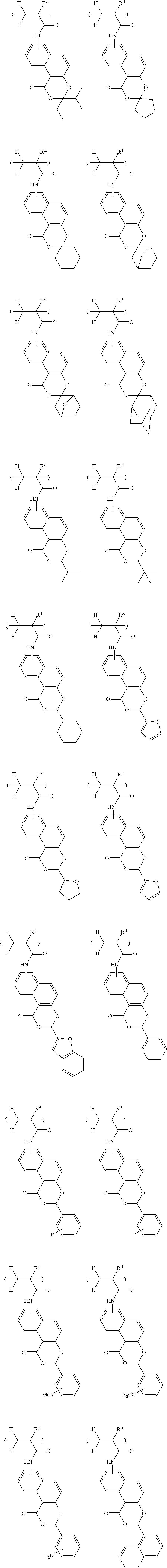

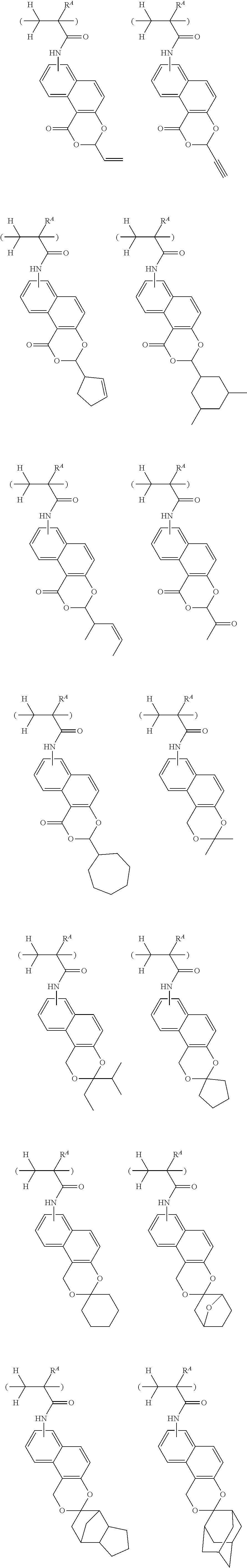

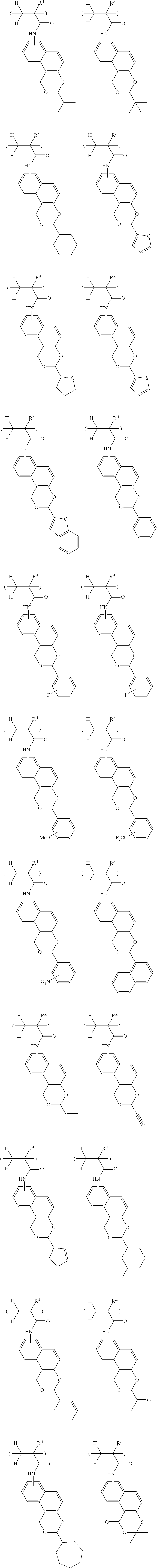

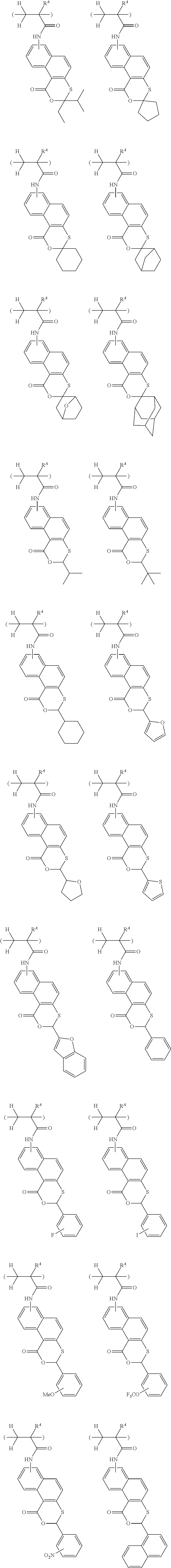

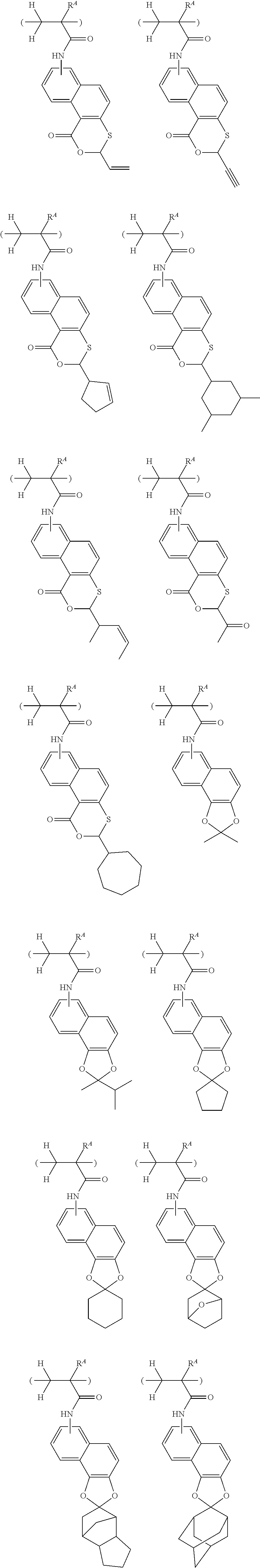

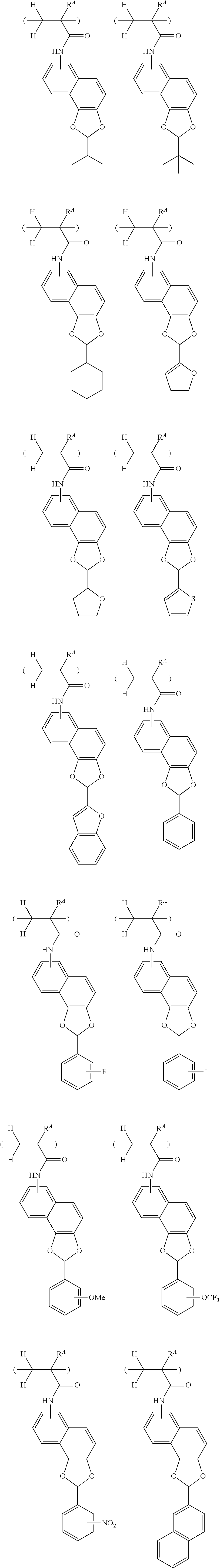

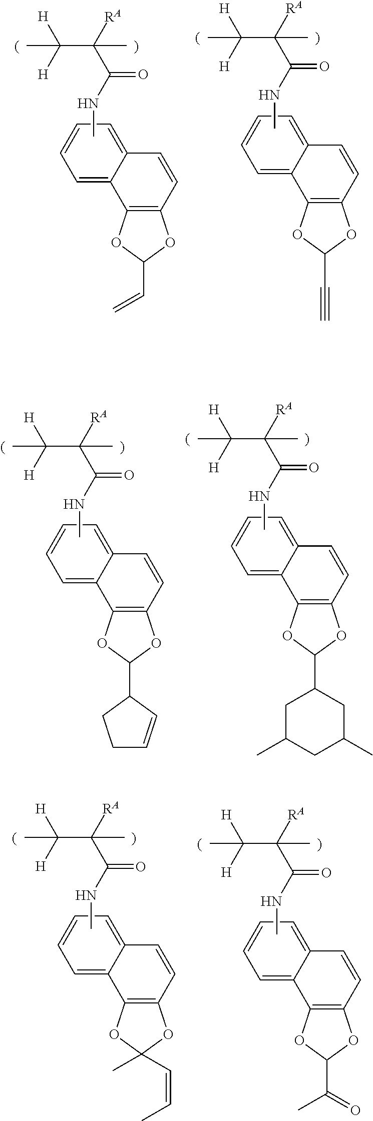

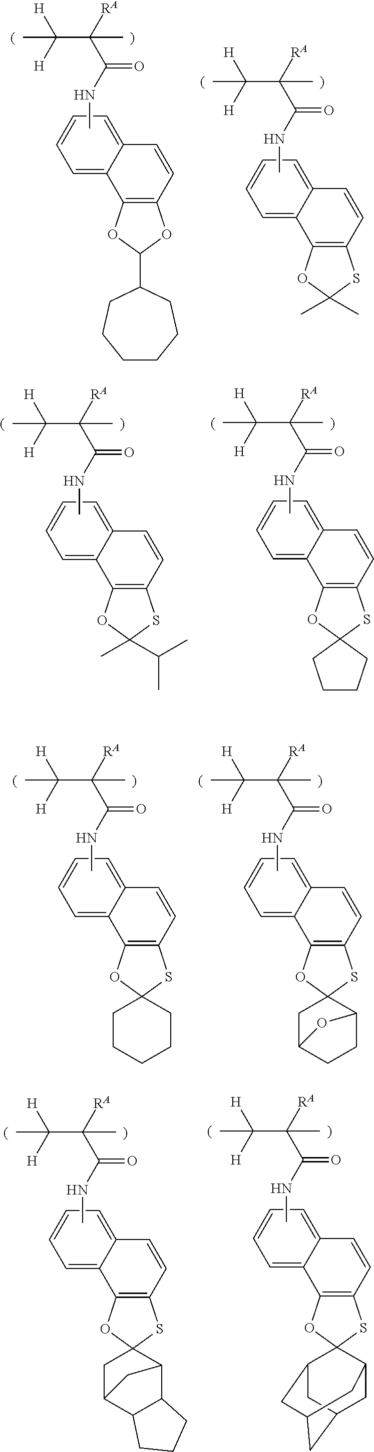

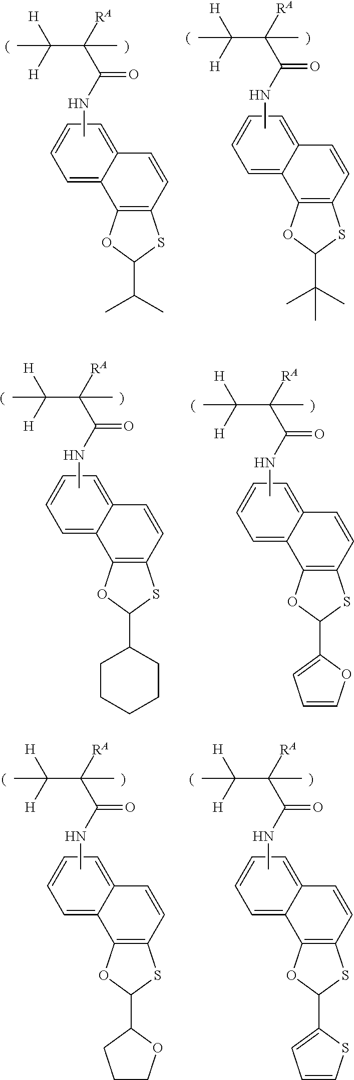

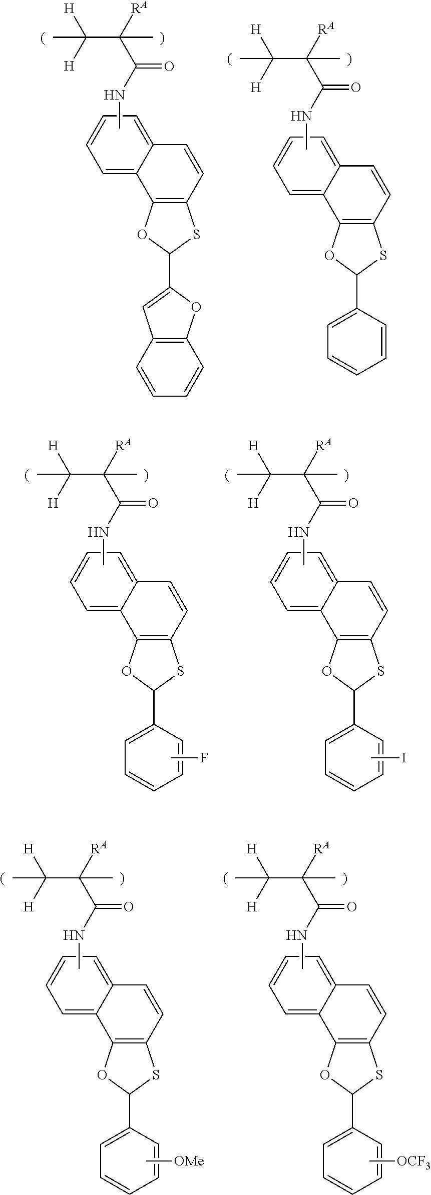

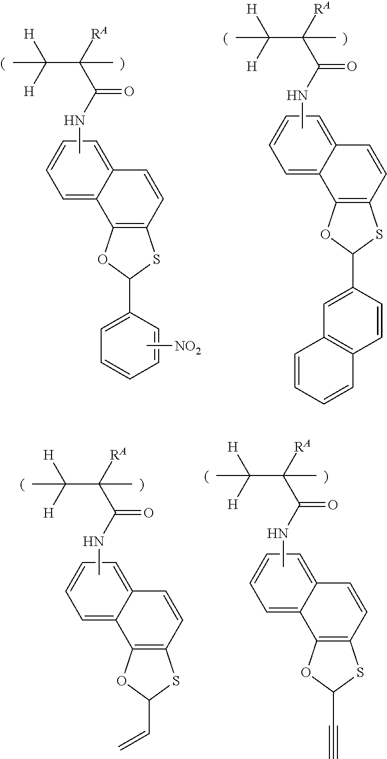

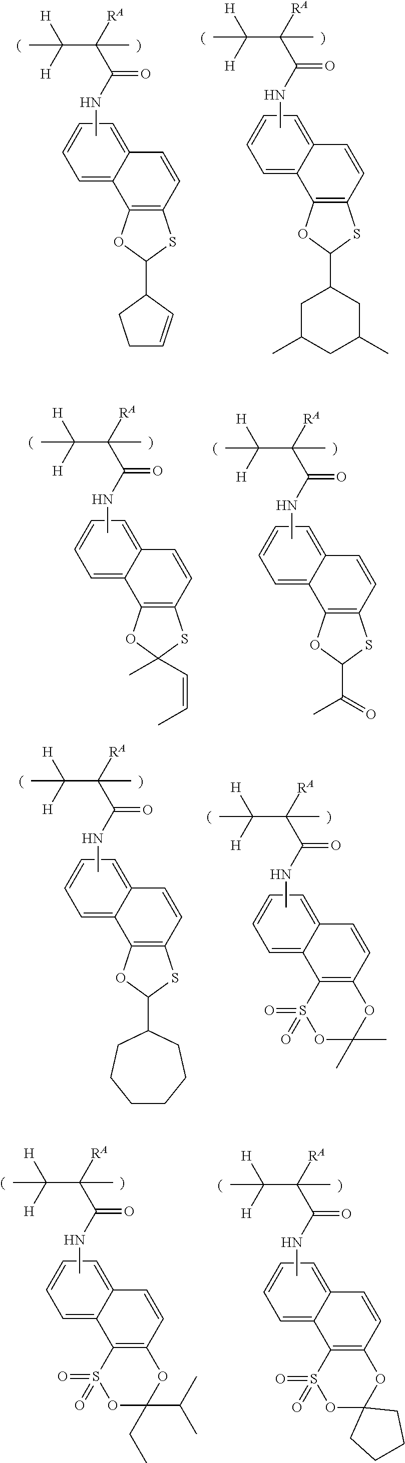

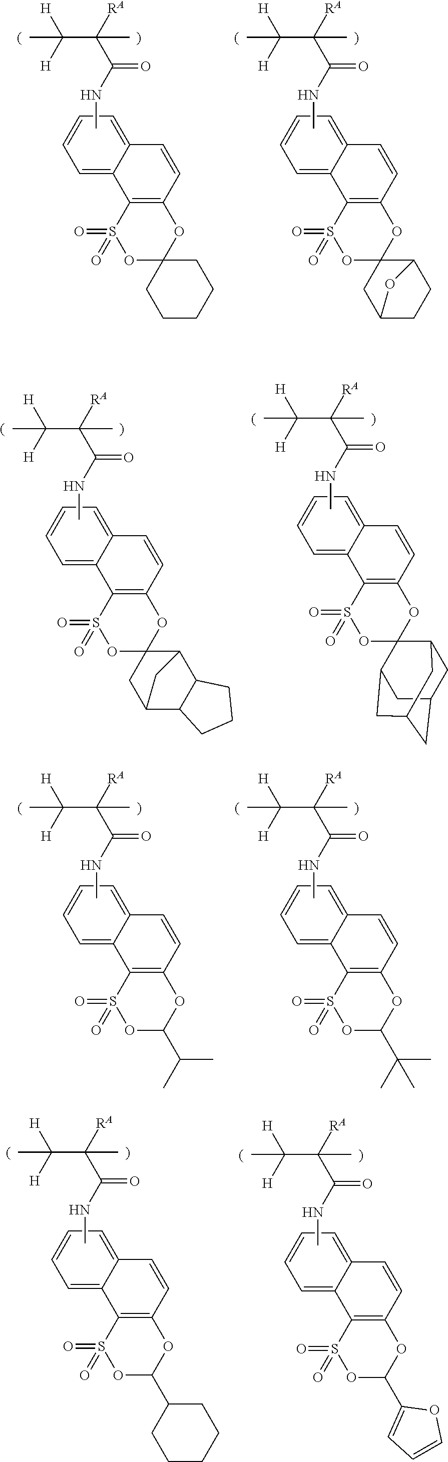

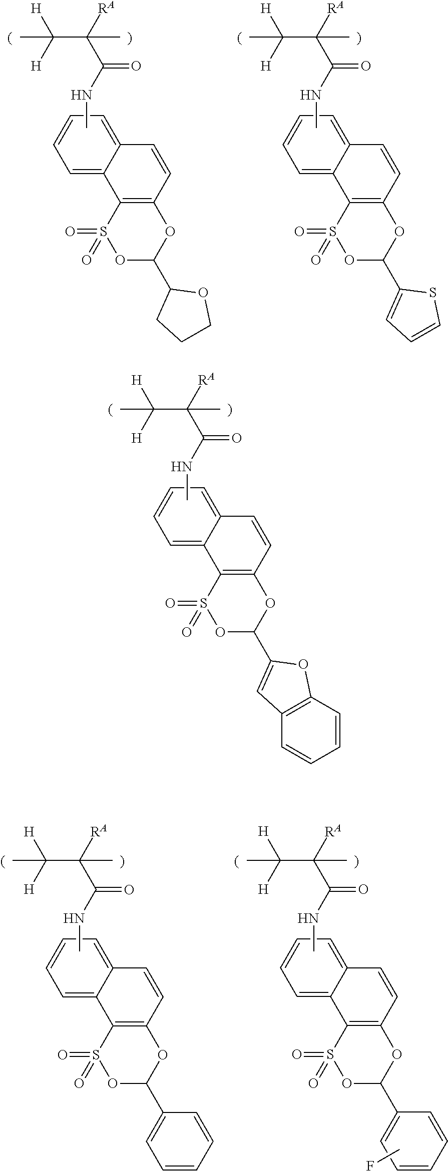

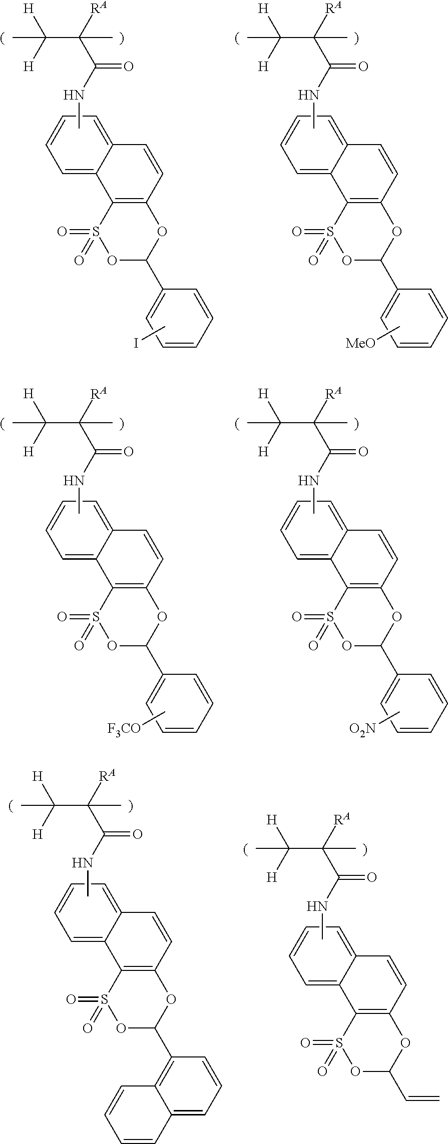

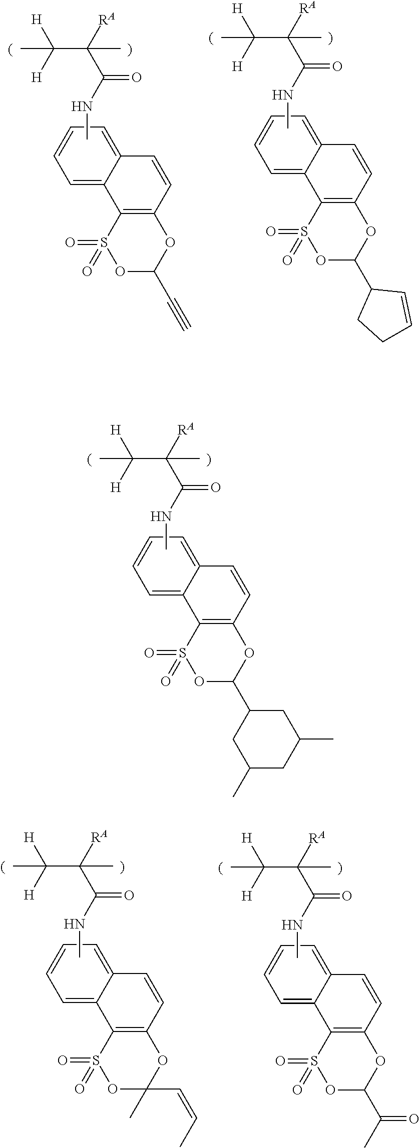

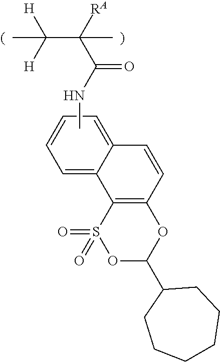

The base polymer preferably comprises repeat units of at least one type selected from repeat units having the formula (b1) (hereinafter, also referred to as repeat units b1) and repeat units having the formula (b2) (hereinafter, also referred to as repeat units b2).

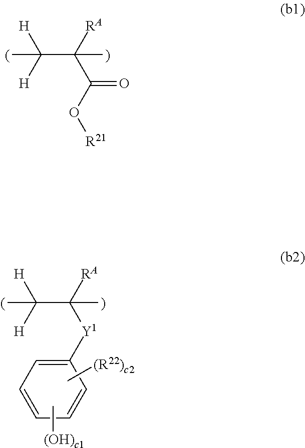

In formulae (b1) and (b2), R^{A} is each independently a hydrogen atom, a fluorine atom, a methyl group, or a trifluoromethyl group. Y¹ is a single bond or *—C(═O)—O—. * designates a point of attachment to the carbon atom in the backbone. R²¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—). R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom. A plurality of R²² may be identical or different when c2 is 2, 3, or 4. c1 is 1, 2, 3, or 4. c2 is 0, 1, 2, 3, or 4. Provided that 1≤c1+c2≤5.

Specific examples of the repeat units b1 are shown below, but are not limited thereto. Herein R^{A} is as defined above.

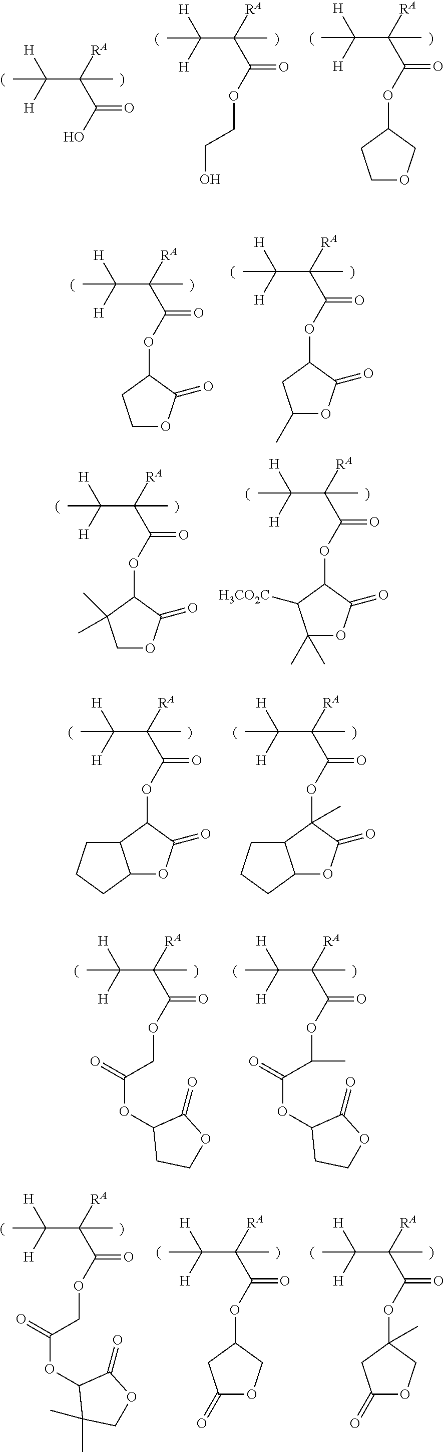

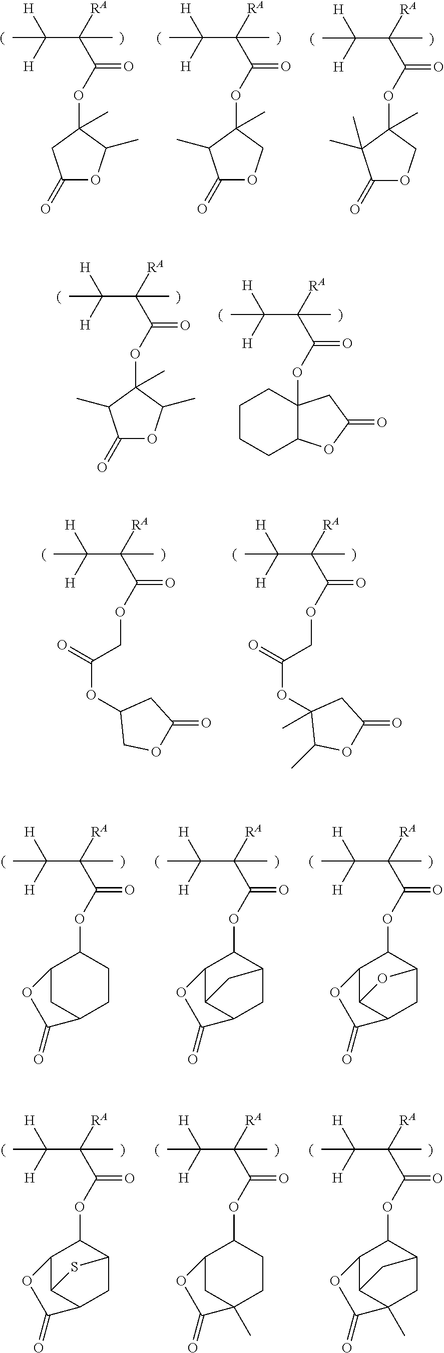

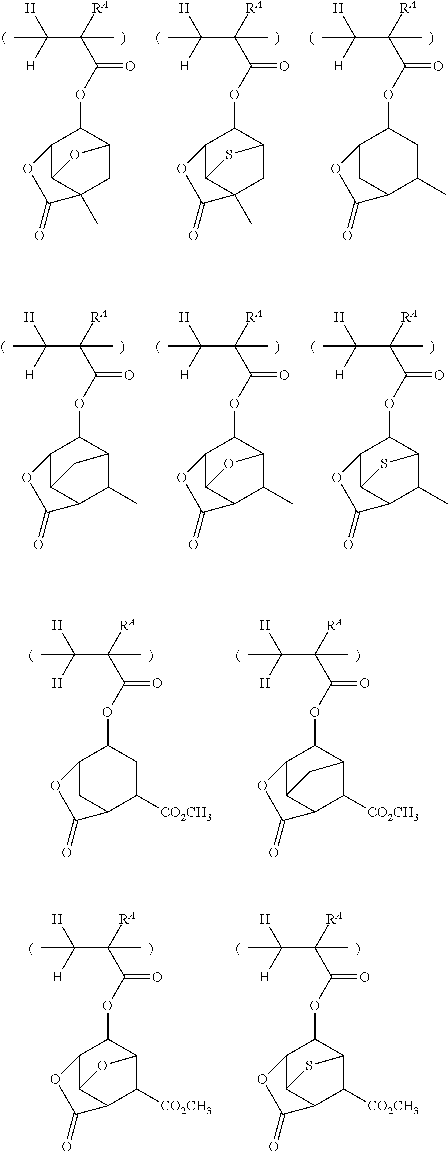

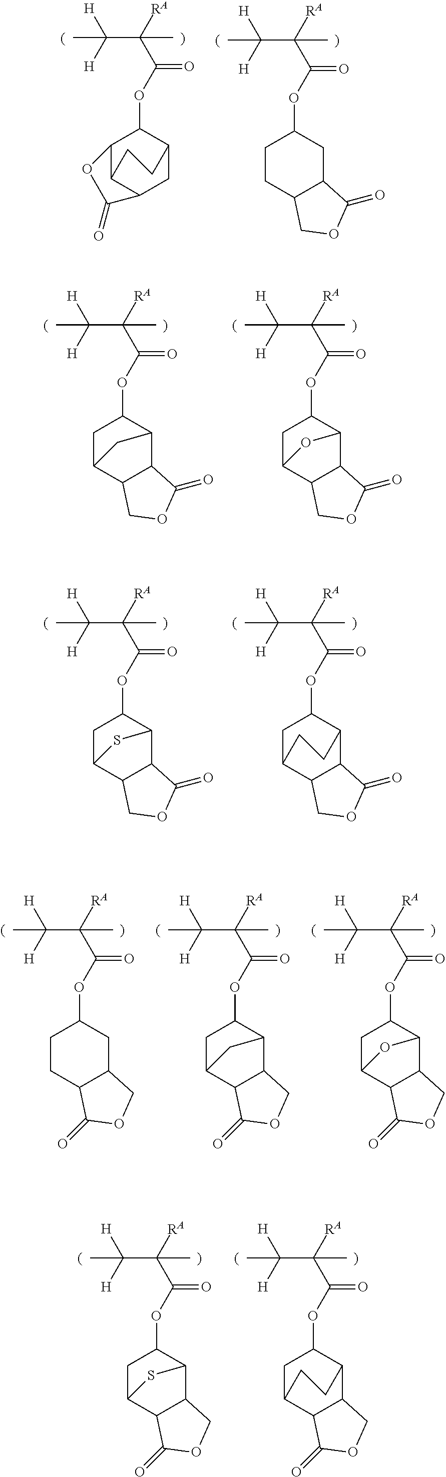

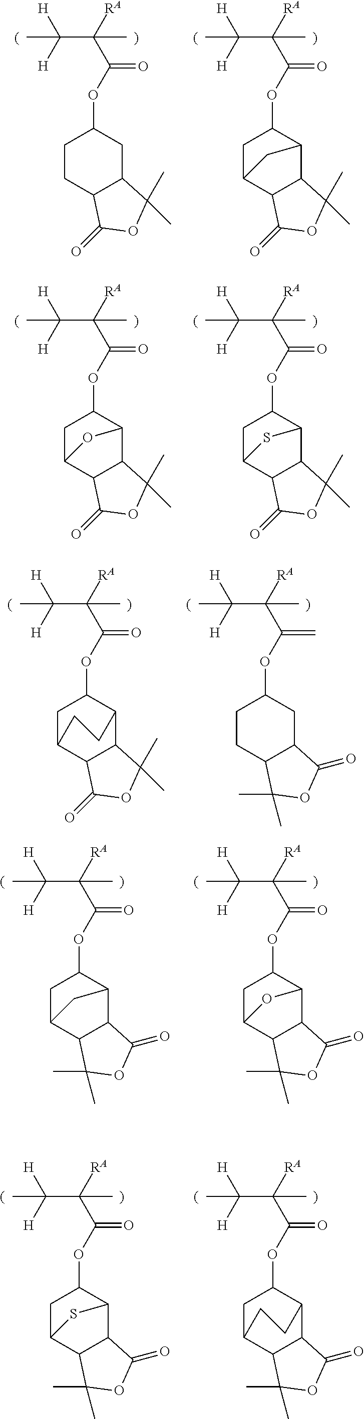

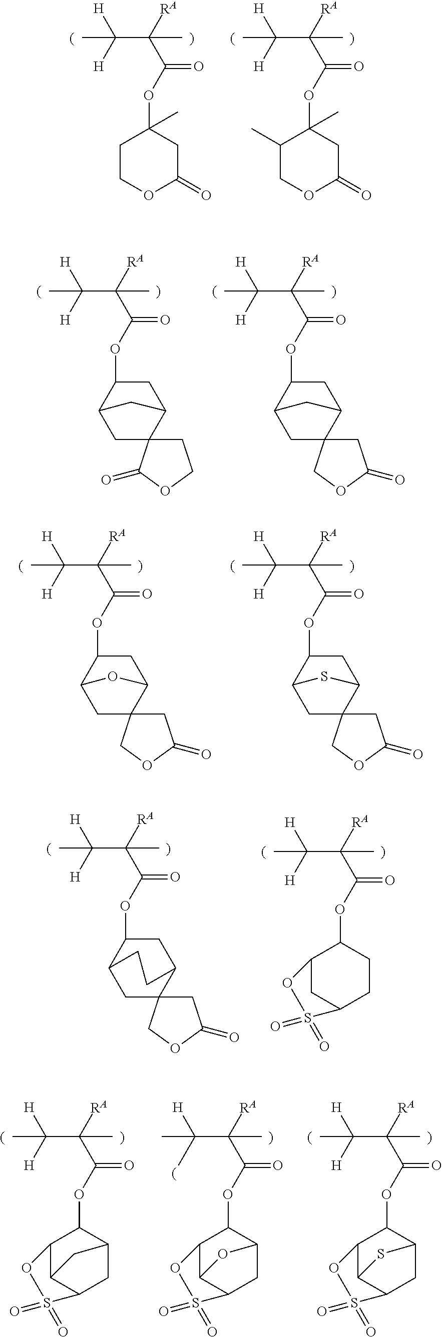

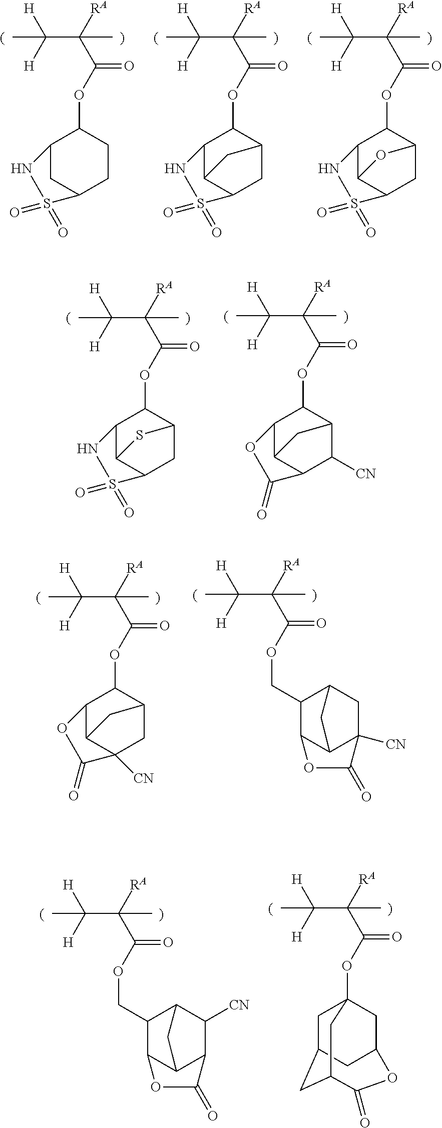

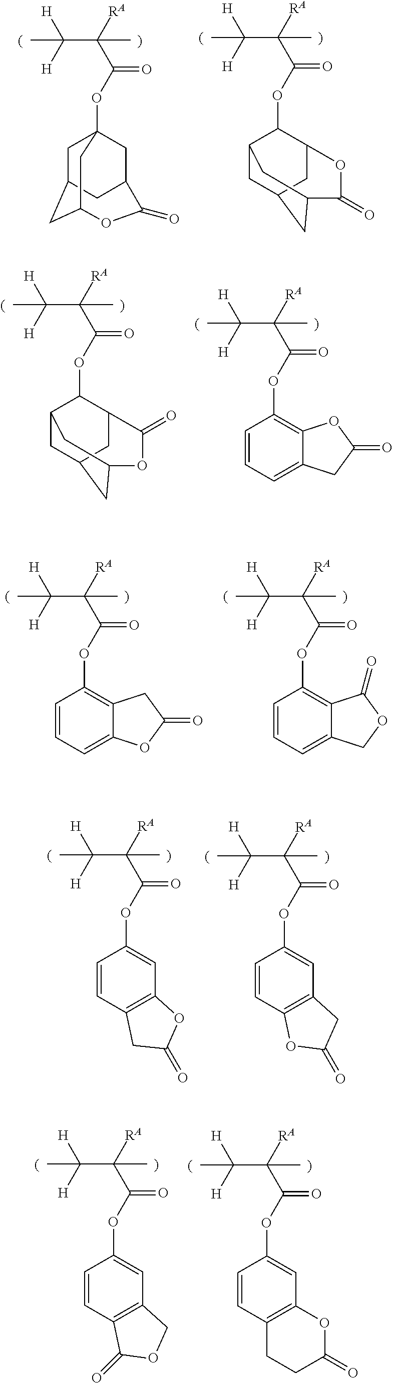

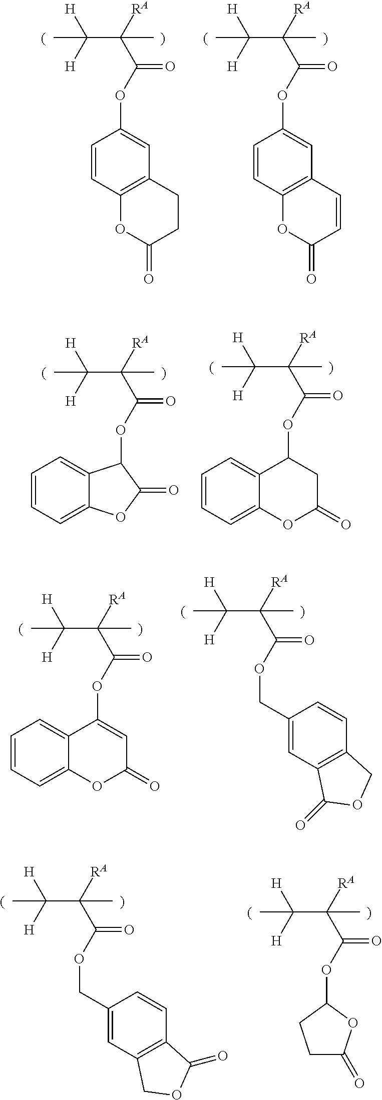

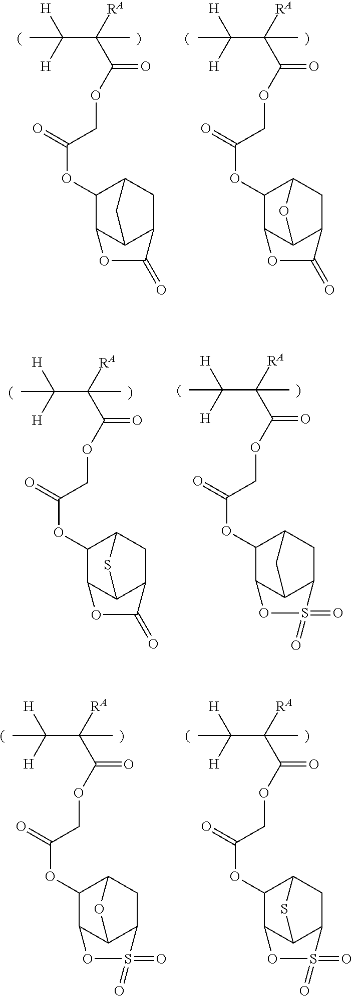

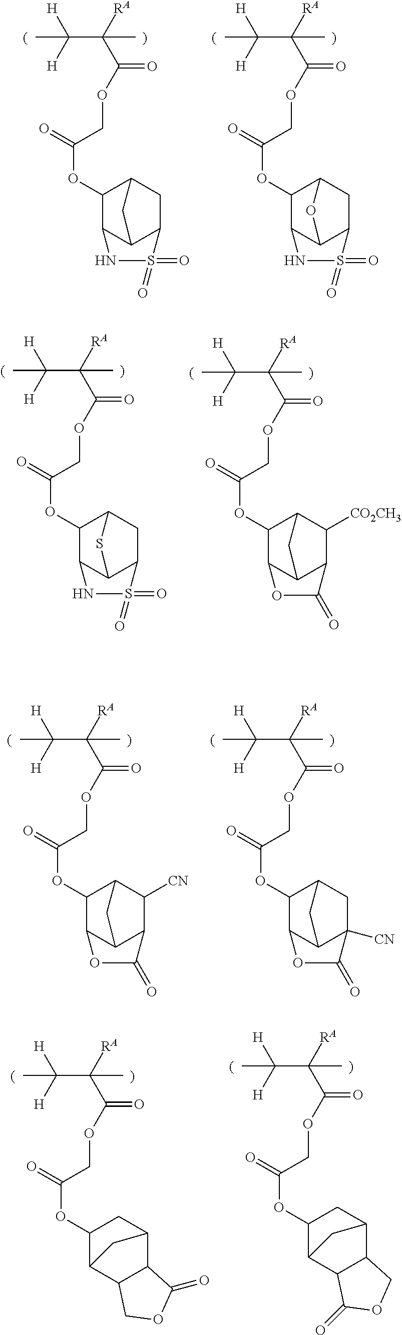

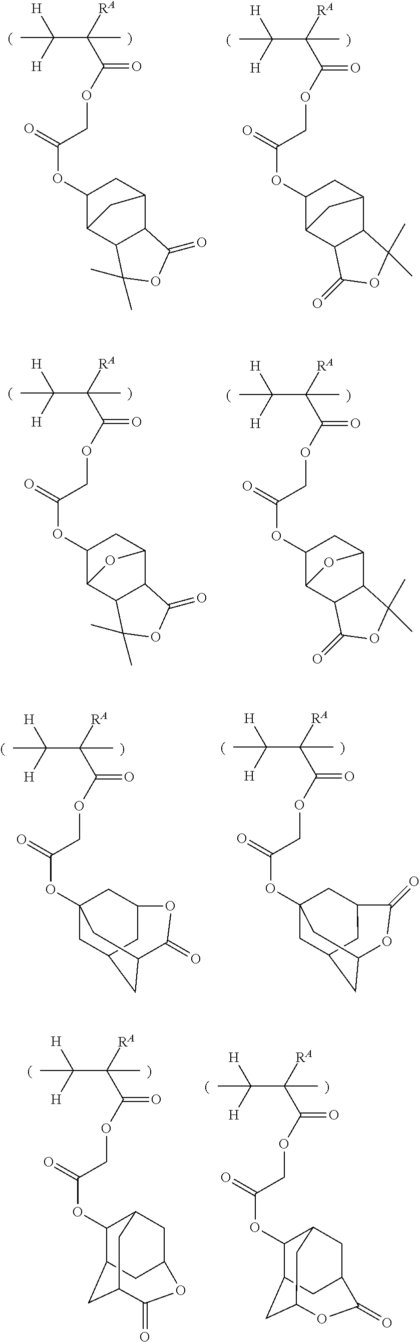

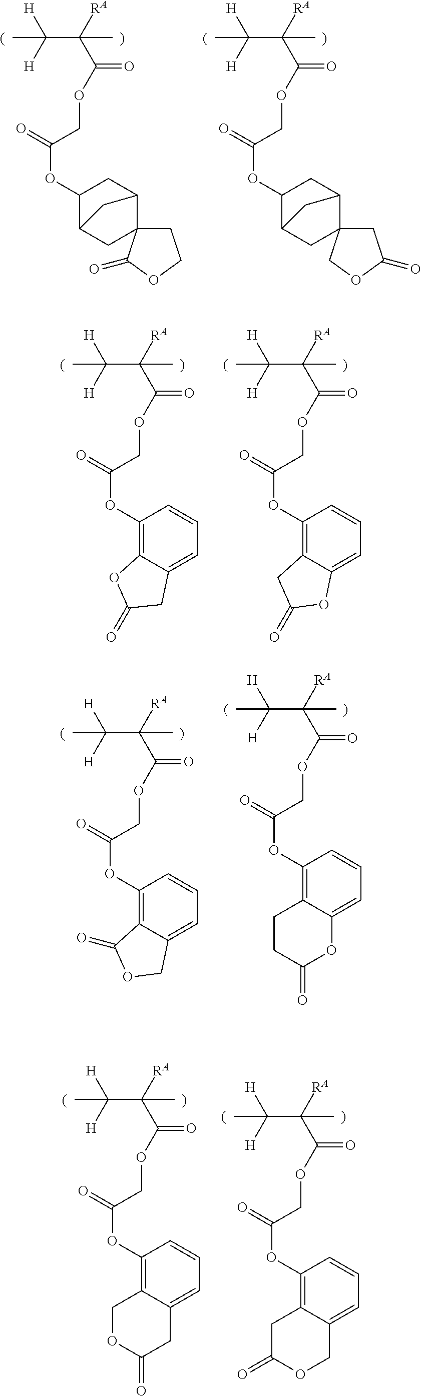

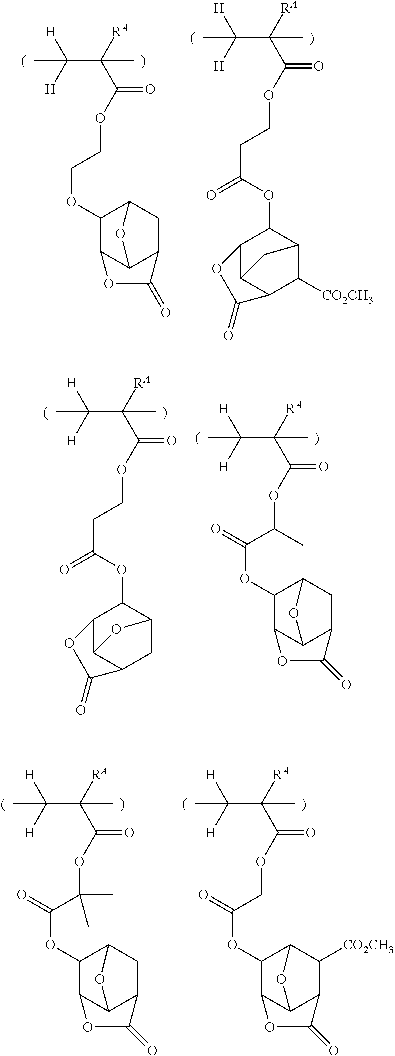

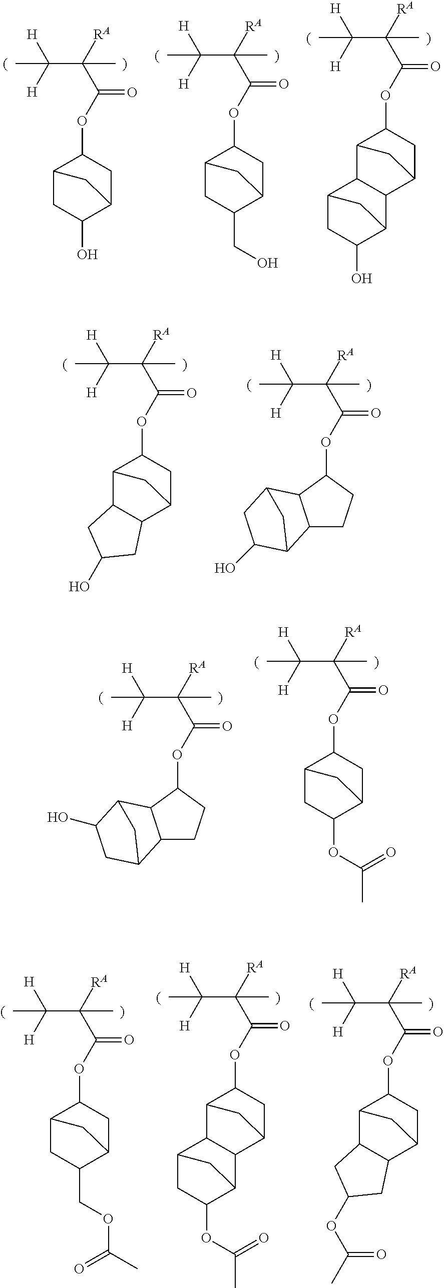

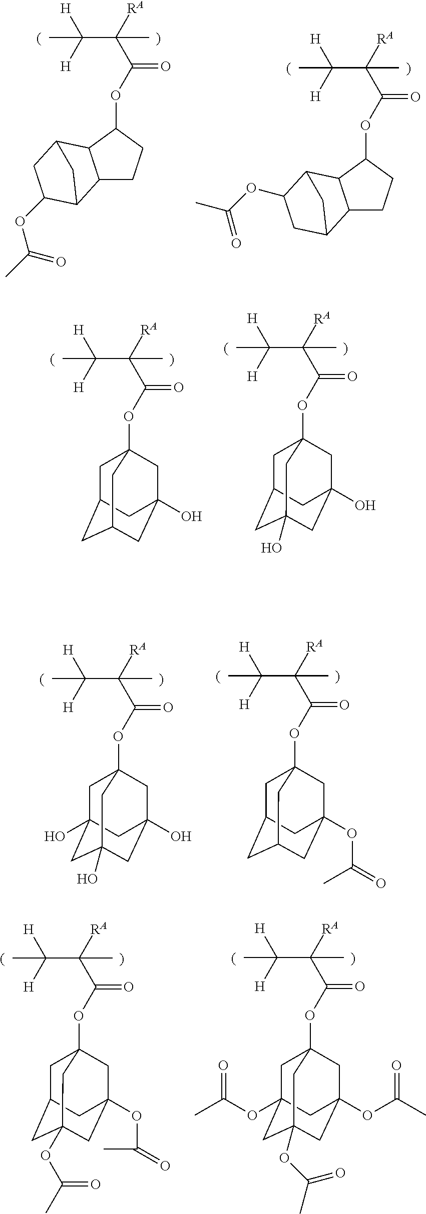

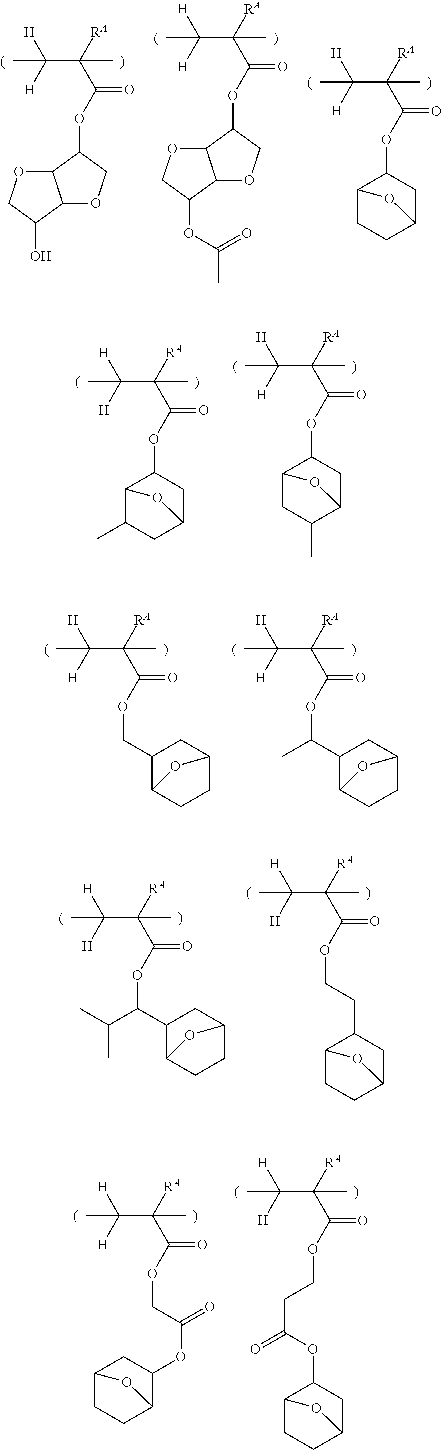

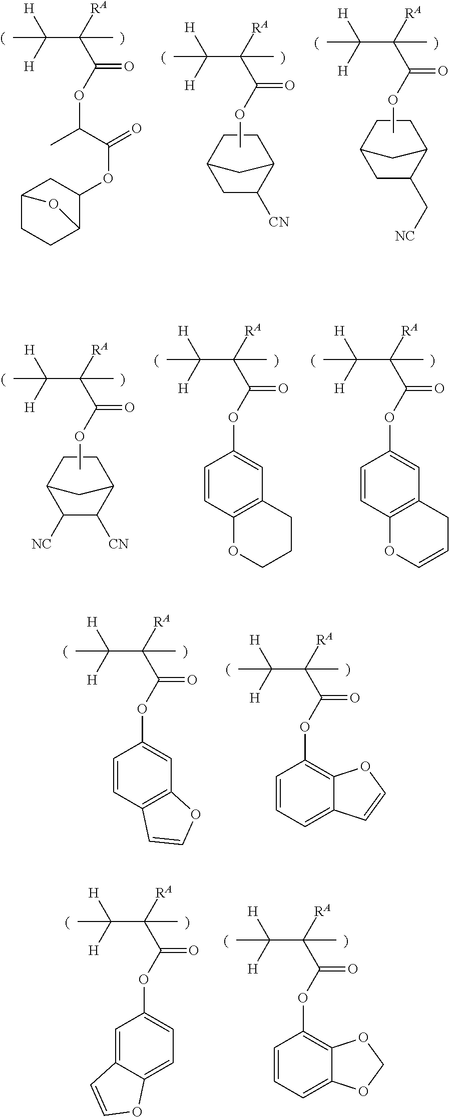

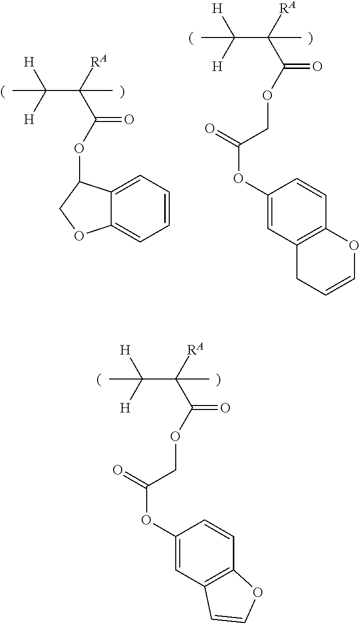

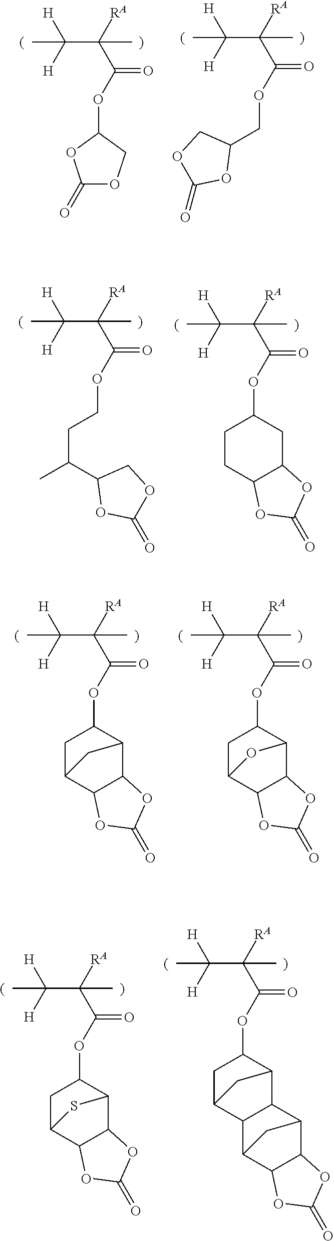

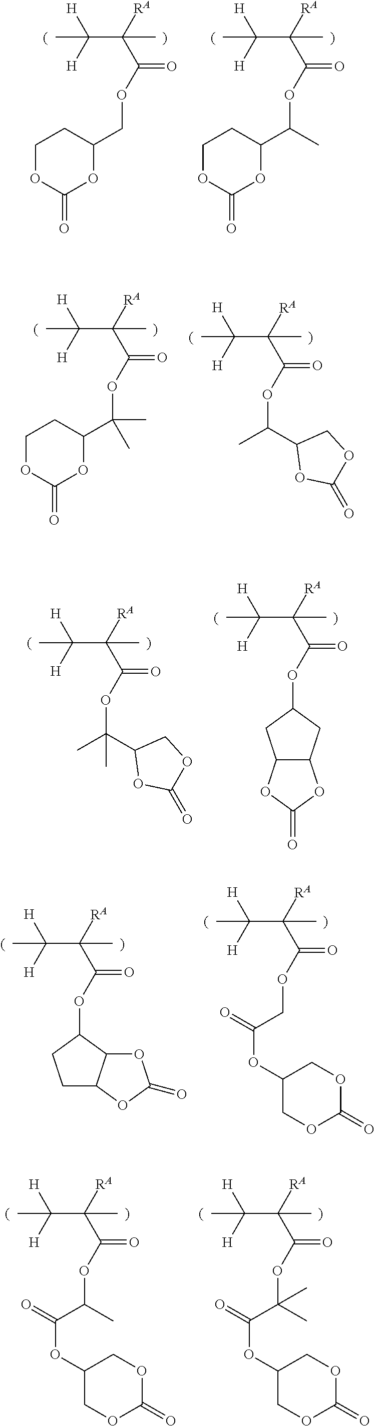

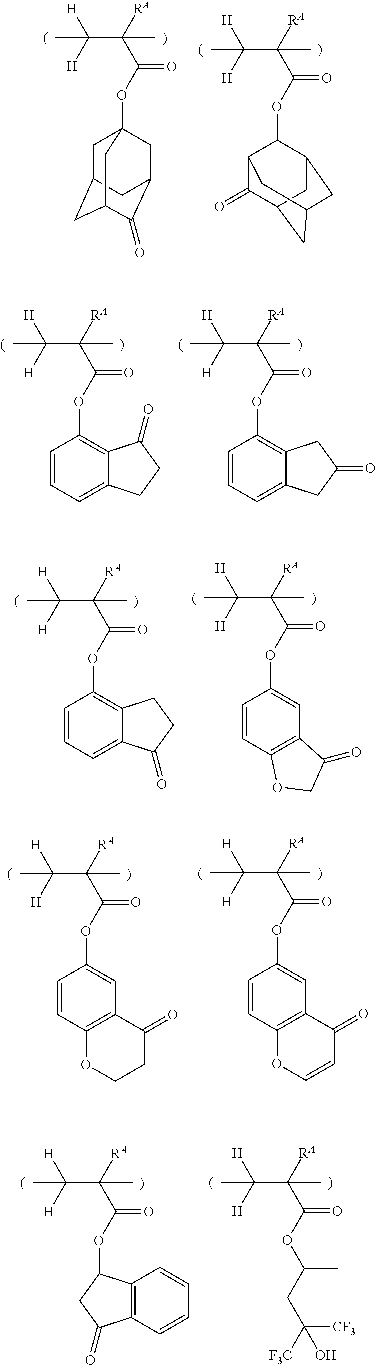

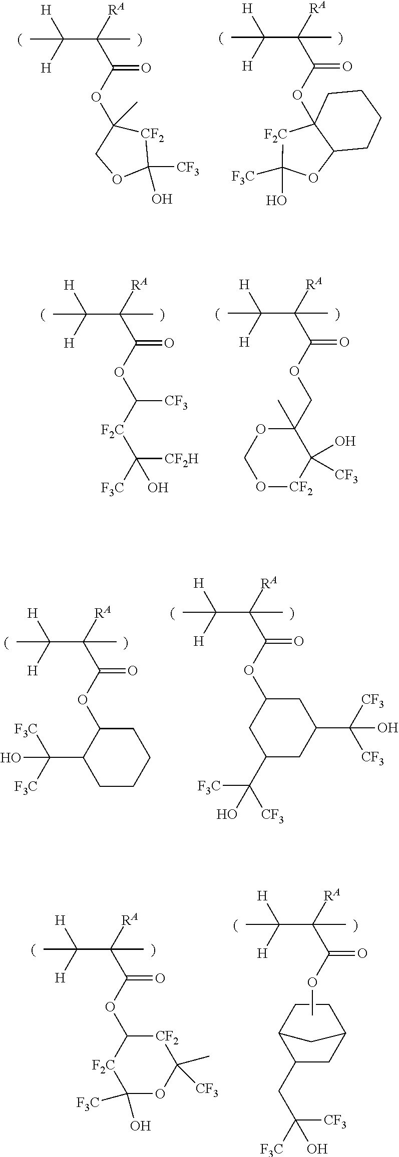

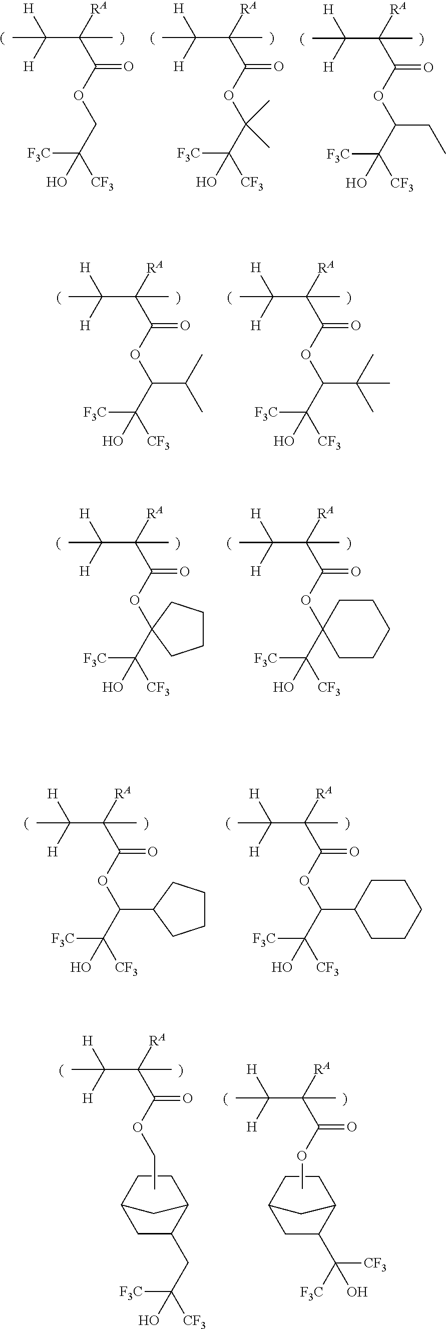

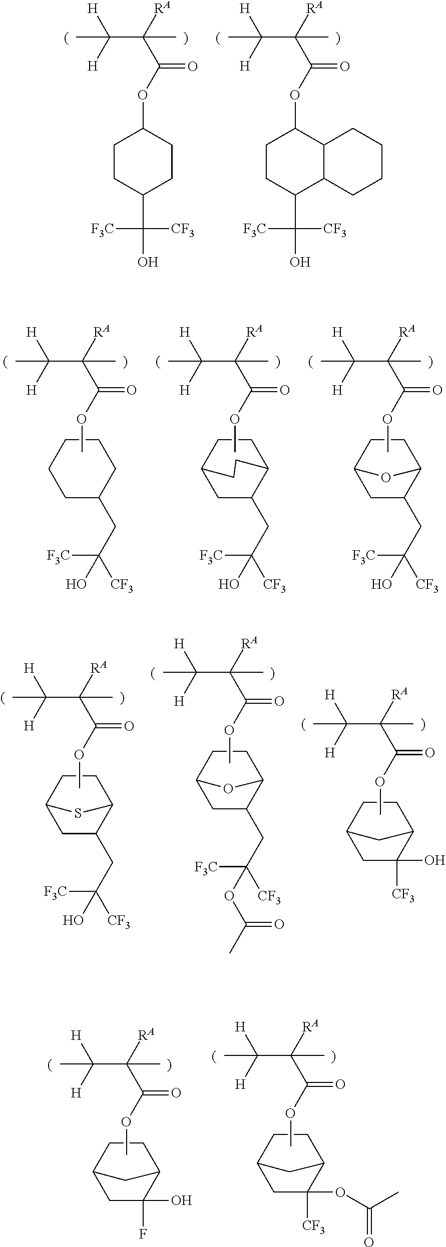

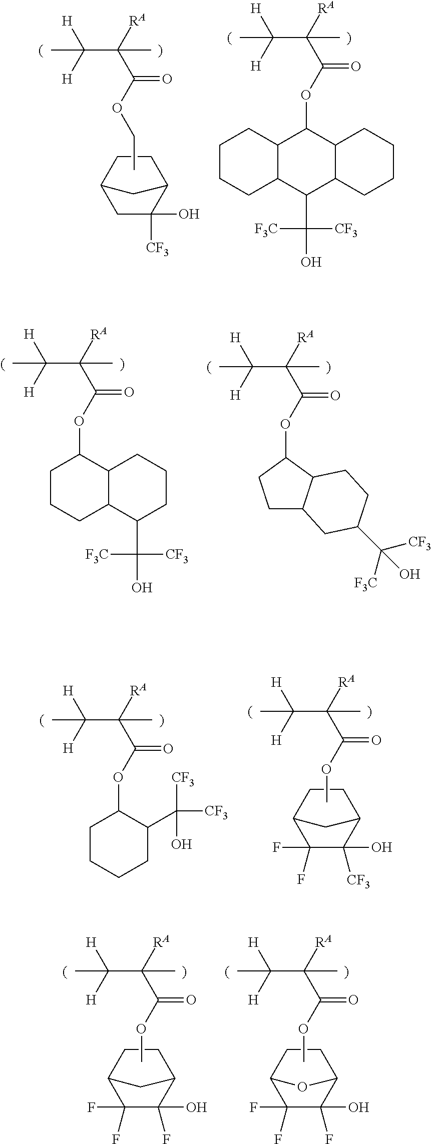

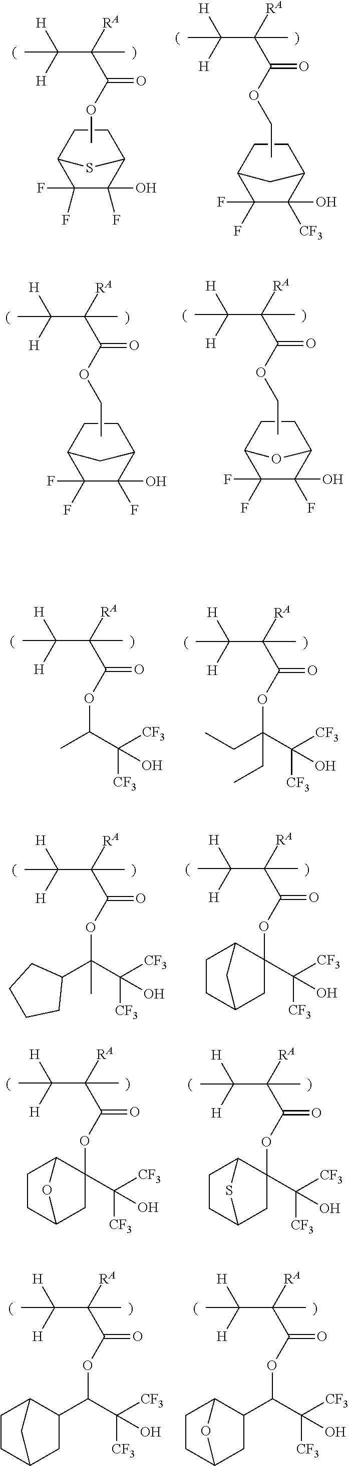

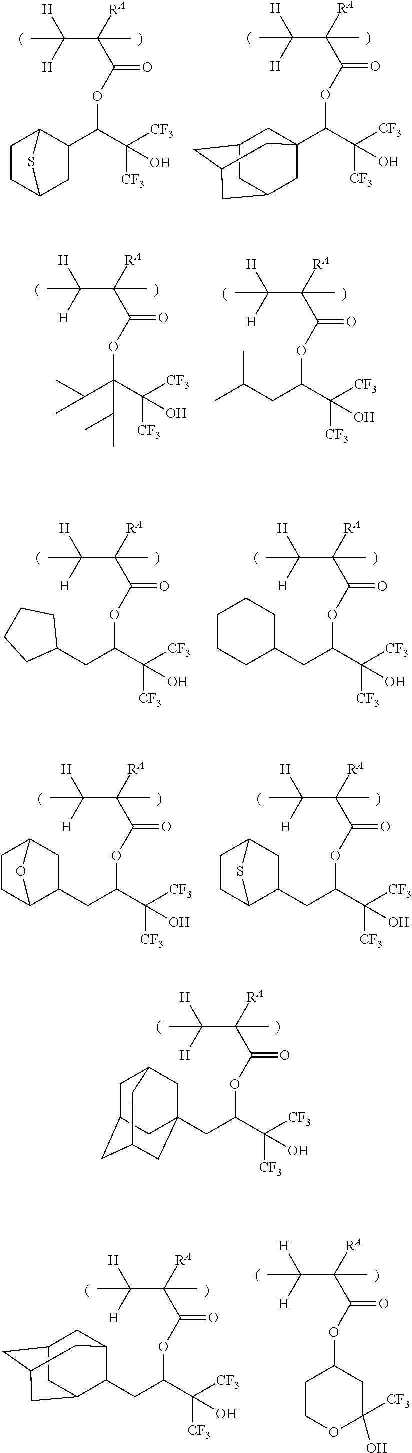

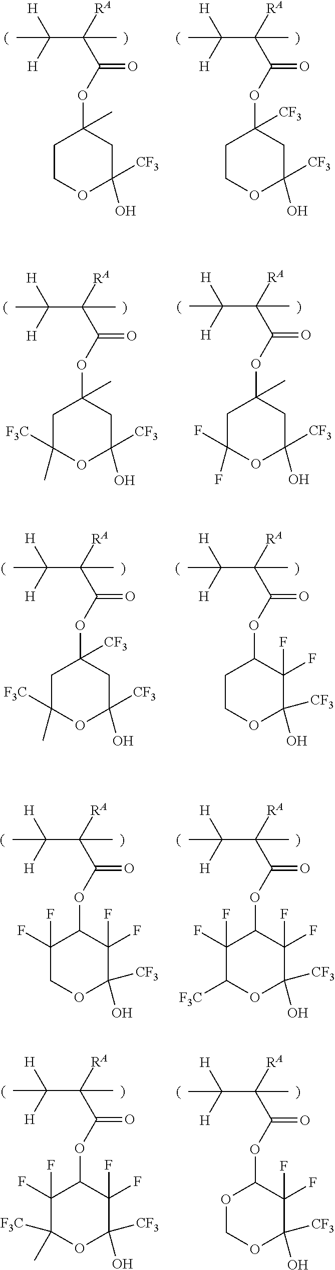

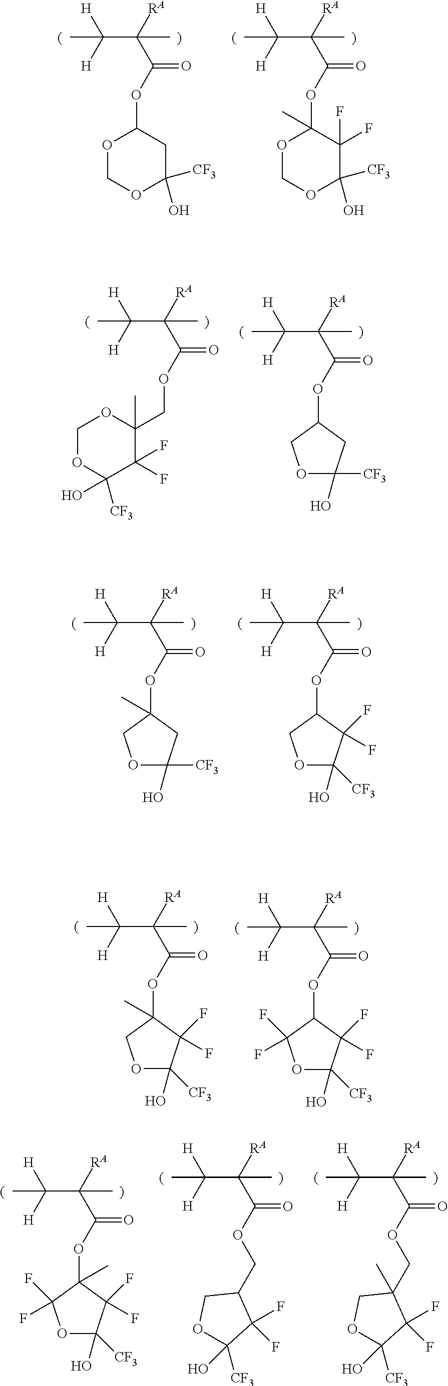

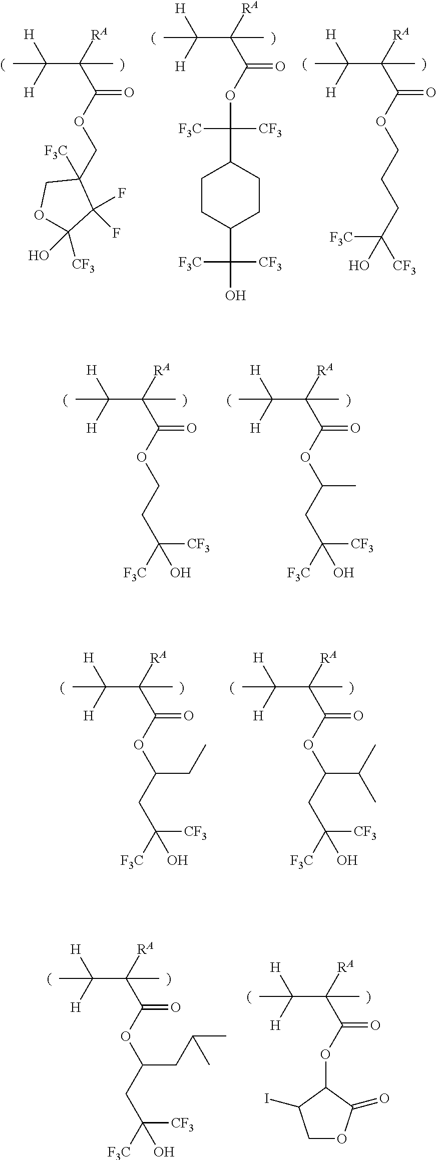

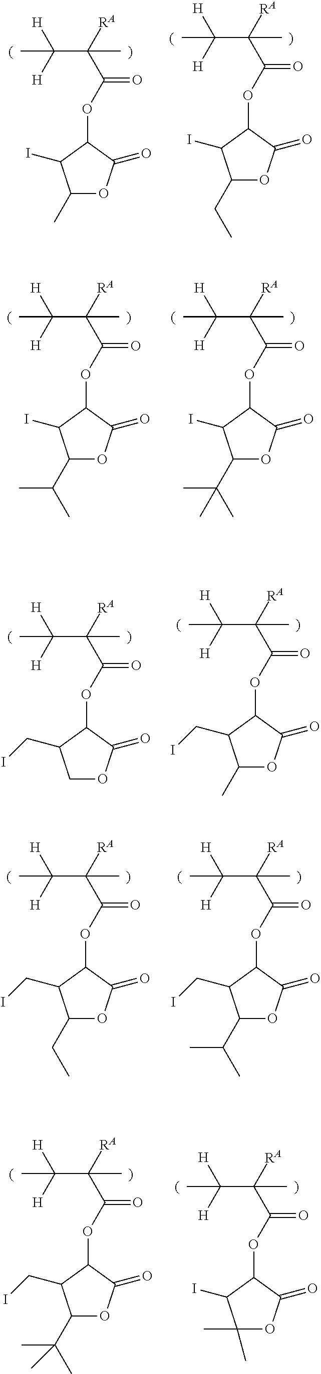

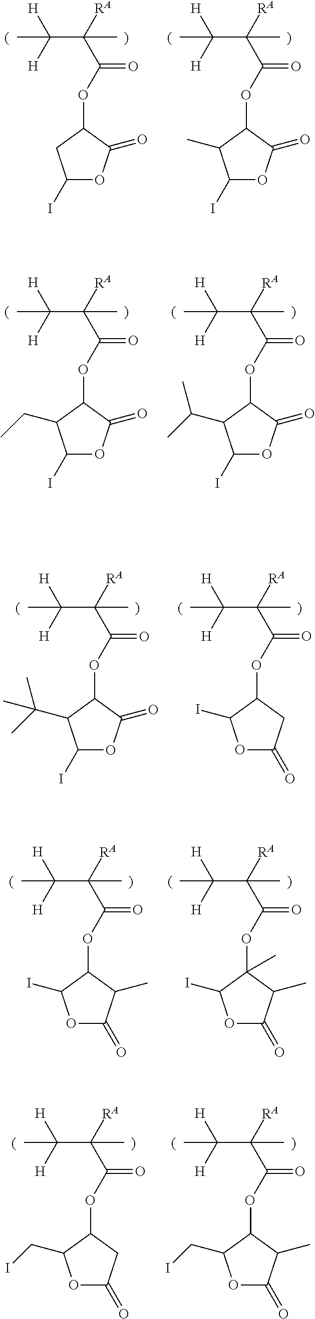

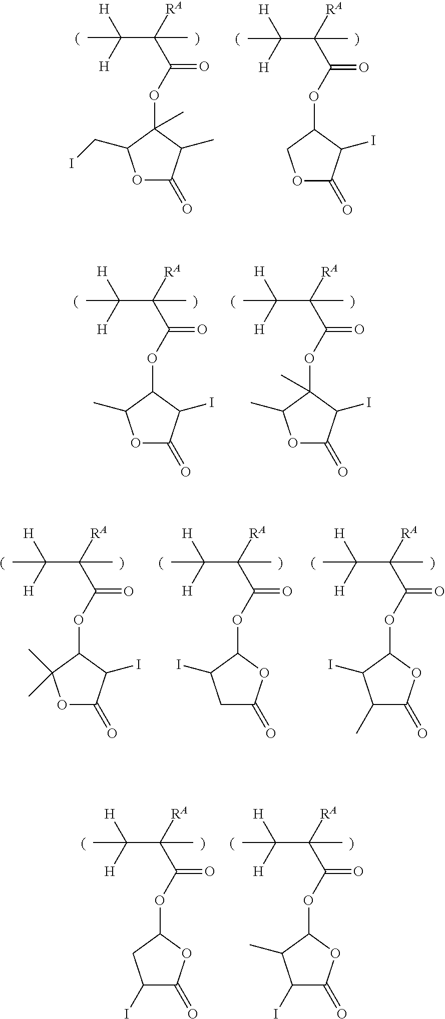

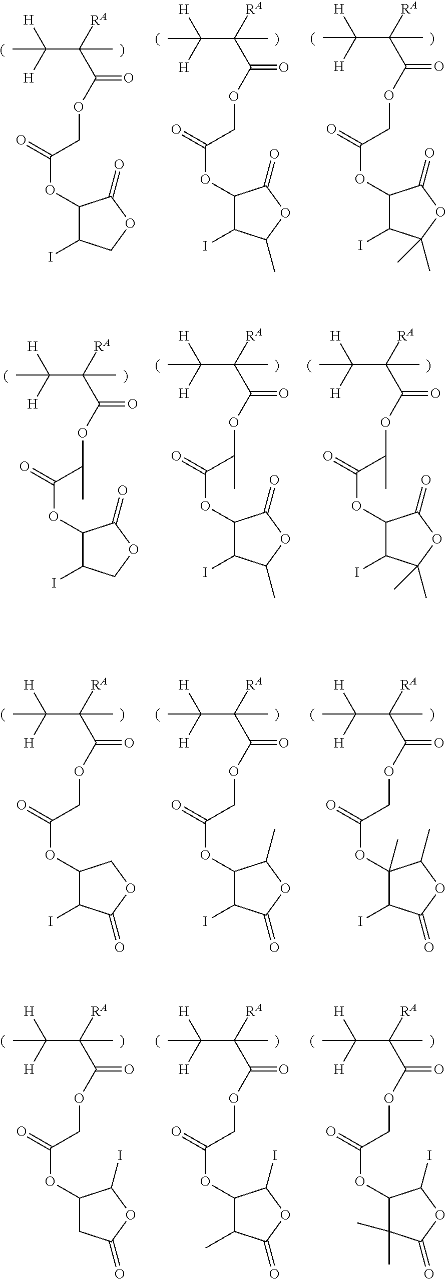

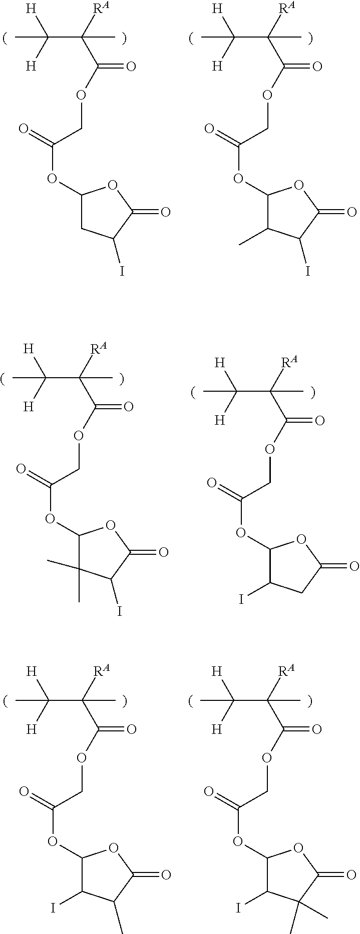

Specific examples of the repeat units b2 are shown below, but are not limited thereto. Herein R^{A} is as defined above.

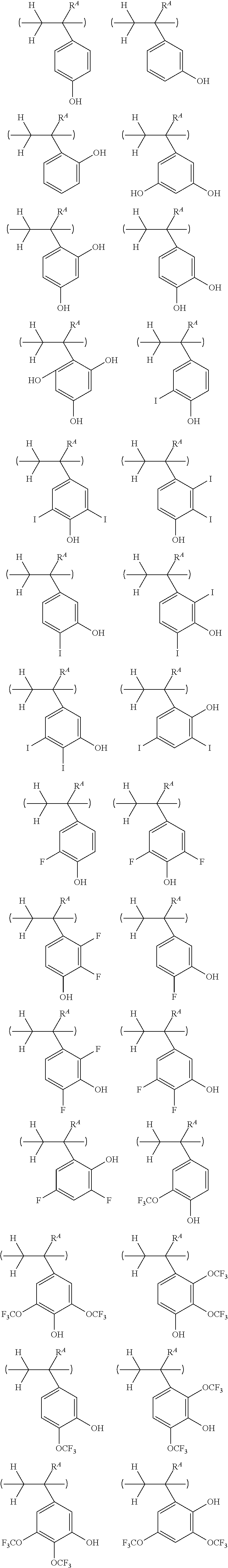

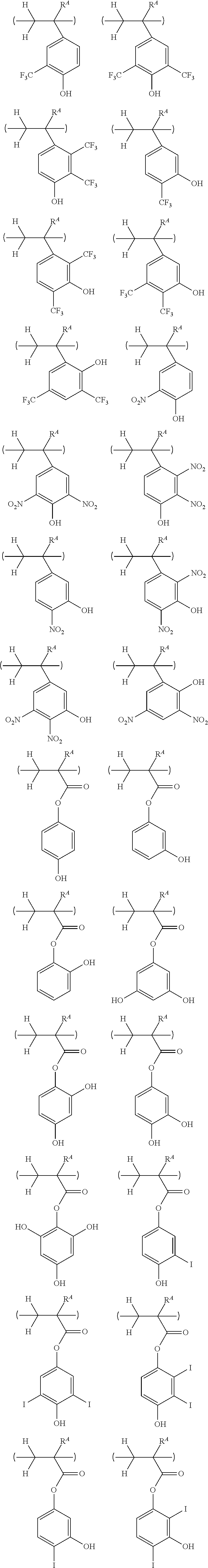

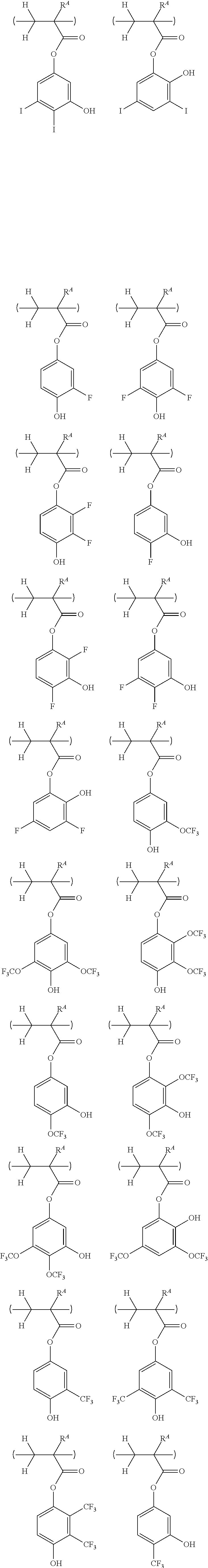

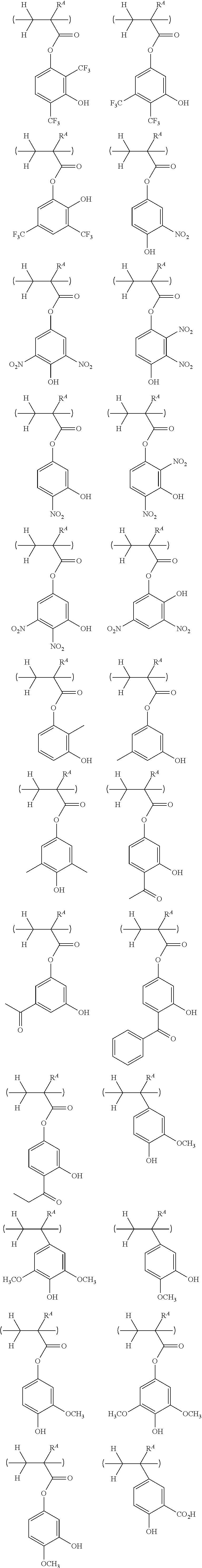

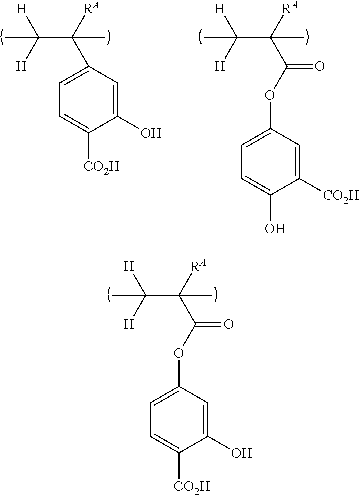

The repeat units b1 or b2 are particularly preferably those units having a lactone ring as the polar group in ArF lithography. and preferably those units having a phenol site in the KrF. EB and EUV lithography.

The base polymer may comprise repeat units of at least one type selected from repeat units having the formula (c1) (hereinafter, also referred to as repeat units c1), repeat units having the formula (c2) (hereinafter, also referred to as repeat units c2), repeat units having the formula (c3) (hereinafter, also referred to as repeat units c3), repeat units having the formula (c4) (hereinafter, also referred to as repeat units c4), and repeat units having the formula (c5) (hereinafter, also referred to as repeat units c5).

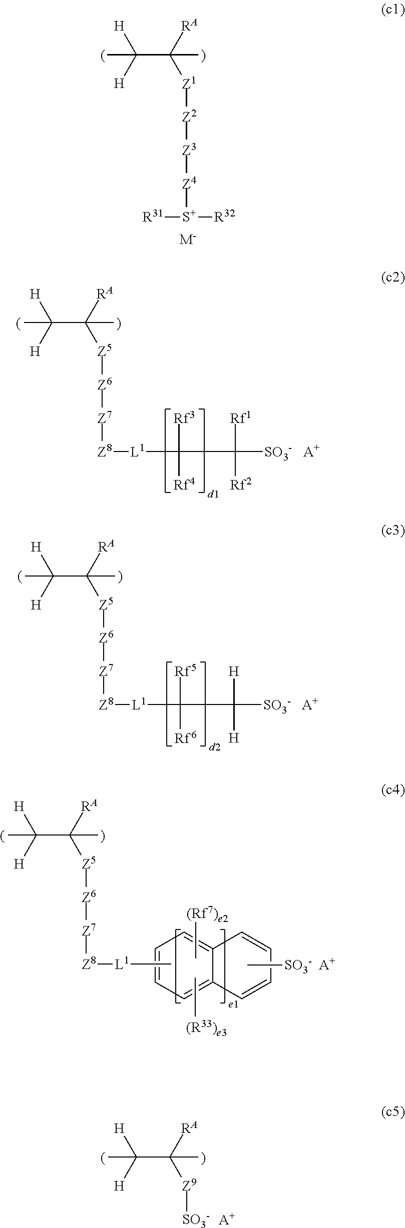

In formulae (c1) to (c5), R^{A} is each independently a hydrogen atom, a fluorine atom, a methyl group, or a trifluoromethyl group. Z¹ is a single bond or an optionally substituted phenylene group. Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining the foregoing, which may contain a halogen atom, a carbonyl group, an ester bond, an ether bond, or a hydroxy group. Z³ is a single bond, an ether bond, an ester bond, an amide bond, a sulfonate ester bond, a sulfonamide bond, a carbonate bond, or a carbamate bond. Z⁴ is a single bond, or a C₁-C₆ aliphatic hydrocarbylene group, a phenylene group, or a divalent group obtained by combining the foregoing, which may contain a halogen atom, a carbonyl group, an ester bond, an ether bond, or a hydroxy group. Z⁵ is each independently a single bond, an optionally substituted phenylene group, a naphthylene group, or *—C(═O)—O—Z⁵¹—. Z⁵¹ is a C₁-C₁₀ aliphatic hydrocarbylene group, a phenylene group, or a naphthylene group which may contain a halogen atom, a hydroxy group, an ether bond, an ester bond, or a lactone ring. Z⁶ is a single bond, an ether bond, an ester bond, an amide bond, a sulfonate ester bond, a sulfonamide bond, a carbonate bond, or a carbamate bond. Z⁷ is each independently a single bond, ***—Z⁷¹—C(═O)—O—, —C(═O)—NH—Z⁷¹—, or ***—O—Z⁷¹—. Z⁷¹ is a C₁-C₂₀ hydrocarbylene group which may contain a heteroatom. Z⁸ is each independently a single bond, ****—Z⁸¹—C(═O)—O—, * ***—C(═O)—NH—Z⁸¹—, or ****—O—Z⁸¹—. Z⁸¹ is a C₁-C₂₀ hydrocarbylene group which may contain a heteroatom. Z° is a single bond, a methylene group, an ethylene group, a phenylene group, a fluorinated phenylene group, a trifluoromethyl-substituted phenylene group, *—C(═O)—O—Z⁹¹—, *—C(═O)—N(H)—Z⁹¹—, or *—O—Z⁹¹—. Z⁹¹ is a C₁-C₆ aliphatic hydrocarbylene group, a phenylene group, a fluorinated phenylene group, or a trifluoromethyl-substituted phenylene group, which may contain a carbonyl group, an ester bond, an ether bond, or a hydroxy group. * designates a point of attachment to the carbon atom in the backbone. ** designates a point of attachment to Z¹. *** designates a point of attachment to Z⁶. **** designates a point of attachment to Z⁷.

The aliphatic hydrocarbylene groups Z²¹, Z⁵¹, and Z⁹¹ may be straight, branched, or cyclic. Specific examples thereof include alkanediyl groups such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-1,1-diyl group, a butane-1,2-diyl group, a butane-1,3-diyl group, a butane-2,3-diyl group, a butane-1,4-diyl group, a 1,1-dimethylethane-1,2-diyl group, a pentane-1,5-diyl group, a 2-methylbutane-1,2-diyl group, and a hexane-1,6-diyl group; cycloalkanediyl groups such as a cyclopropanediyl group, a cyclobutanediyl group, a cyclopentanediyl group, and a cyclohexanediyl group; and combinations thereof.

The hydrocarbylene group which may contain a heteroatom, represented by Z⁷¹ and Z⁸¹, may be saturated or unsaturated and straight, branched, or cyclic. Specific examples thereof are shown below, but are not limited thereto.

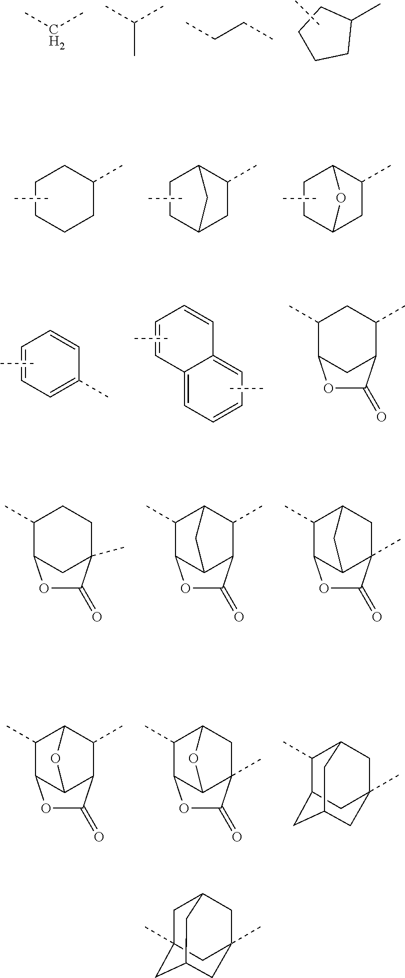

Herein the broken line designates a point of attachment.

In formula (c1), R³¹ and R³² are each independently a C₁-C₂₀ hydrocarbyl group which may contain a heteroatom. The hydrocarbyl group may be saturated or unsaturated and straight, branched, or cyclic. Specific examples thereof include C₁-C₂₀ alkyl groups such as a methyl group, an ethyl group, an n-propyl group, an isopropyl group, an n-butyl group, an isobutyl group, a sec-butyl group, and a tert-butyl group; C₃-C₂₀ Cyclic saturated hydrocarbyl groups such as a cyclopropyl group, a cyclopentyl group, a cyclohexyl group, a cyclopropylmethyl group, a 4-methylcyclohexyl group, a cyclohexylmethyl group, a norbornyl group, and an adamantyl group; C₂-C₂₀ alkenyl groups such as a vinyl group, a 1-propenyl group, a 2-propenyl group, a butenyl group, and a hexenyl group; C₃-C₂₀ cyclic unsaturated hydrocarbyl groups such as a cyclohexenyl group; C₆-C₂₀ aryl groups such as a phenyl group, a naphthyl group, and a thienyl group; C₇-C₂₀ aralkyl groups such as a benzyl group, a 1-phenylethyl group, and a 2-phenylethyl group; and combinations thereof, and aryl groups are preferred. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

R³¹ and R³² may bond together to form a ring with the sulfur atom to which they are attached. Specific examples of the ring are shown below.

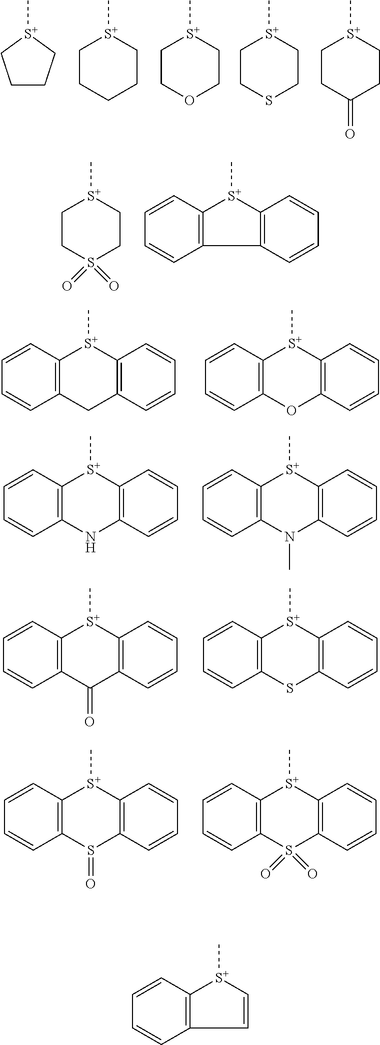

Herein the broken line designates a point of attachment to Z⁴.

Specific examples of the cation of the repeat units e1 are shown below, but are not limited thereto. Herein R^{A} is as defined above.

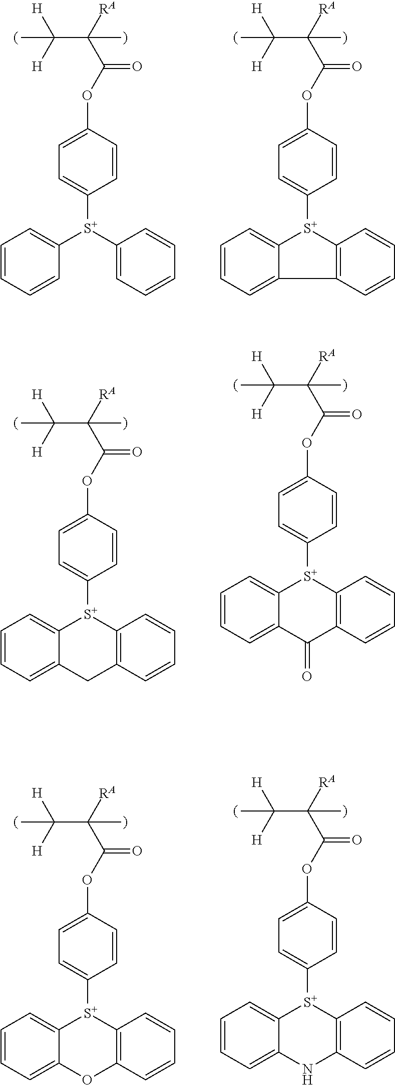

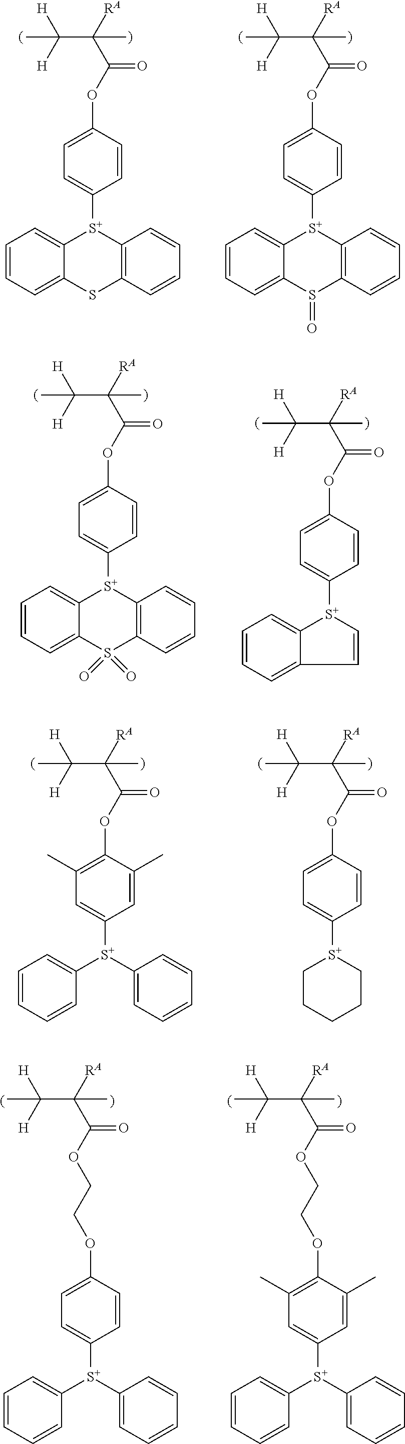

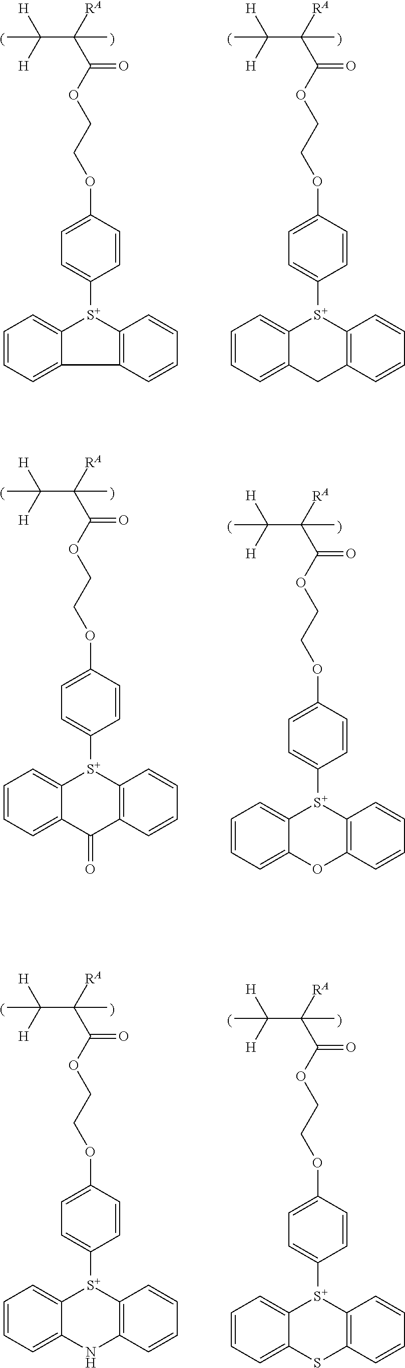

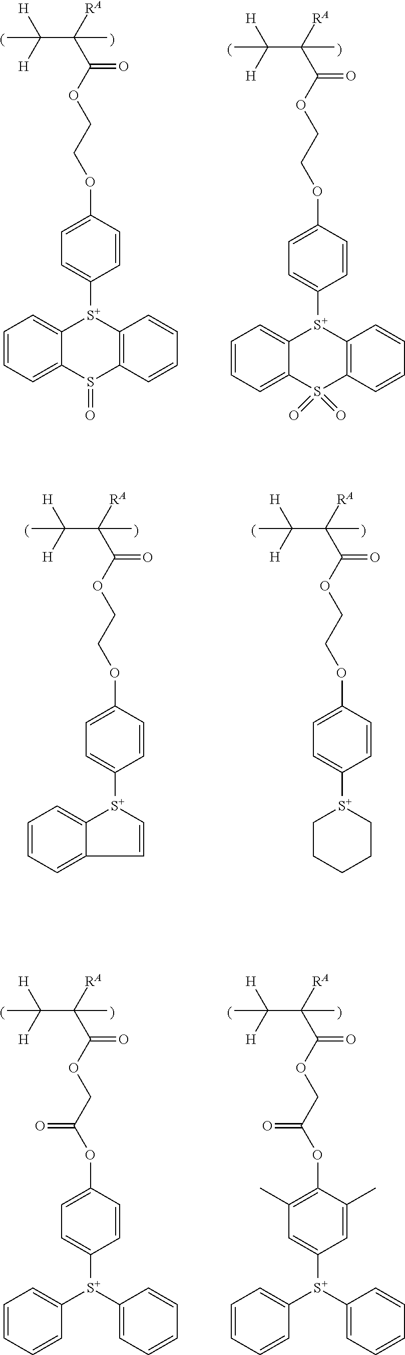

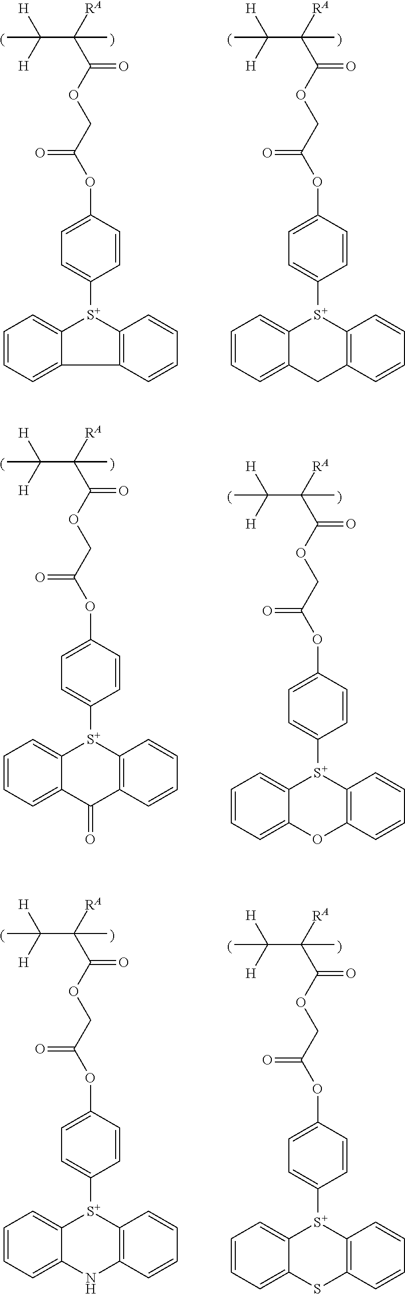

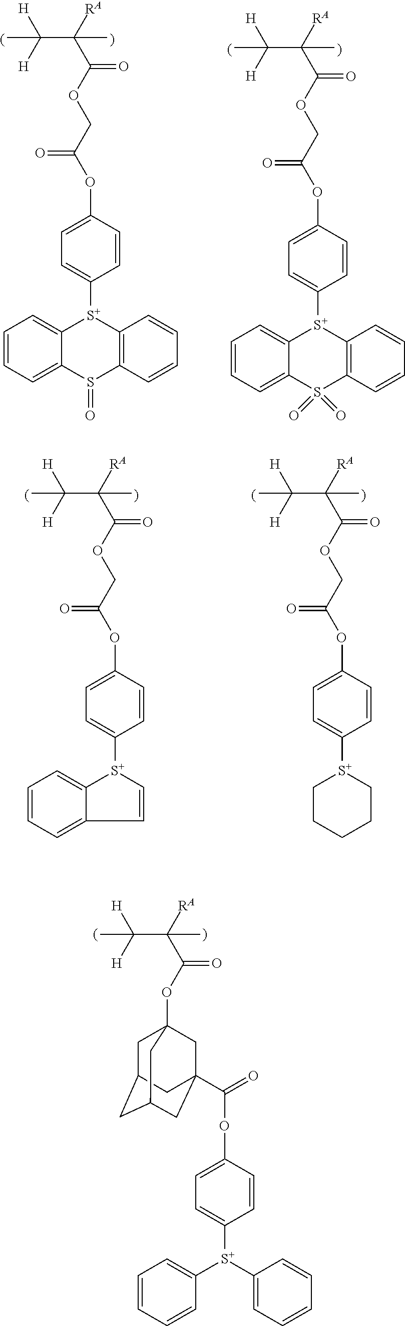

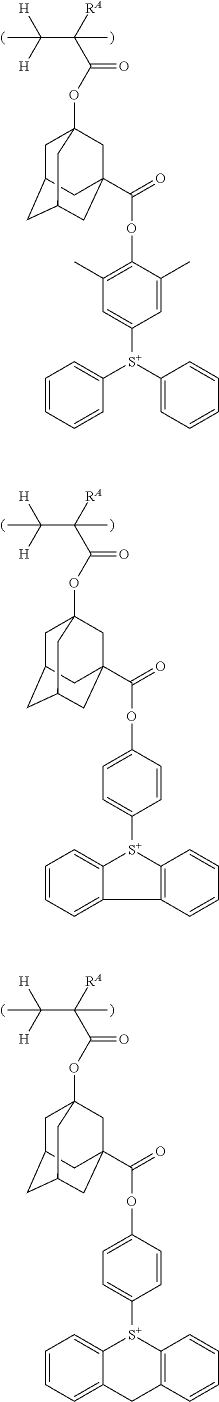

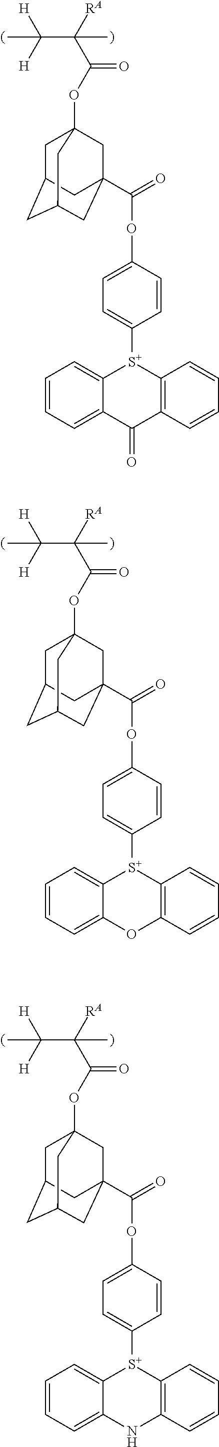

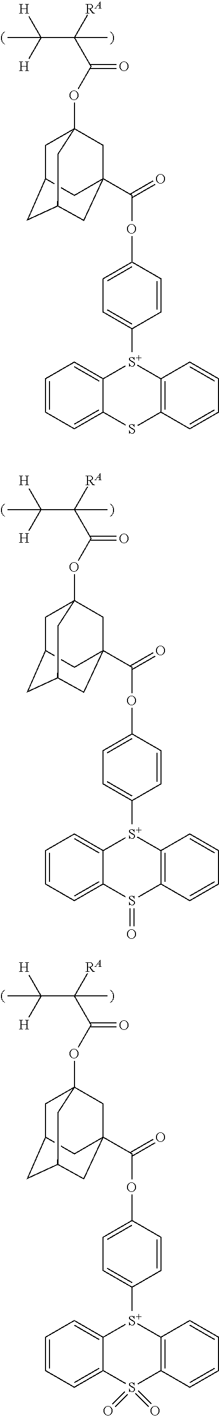

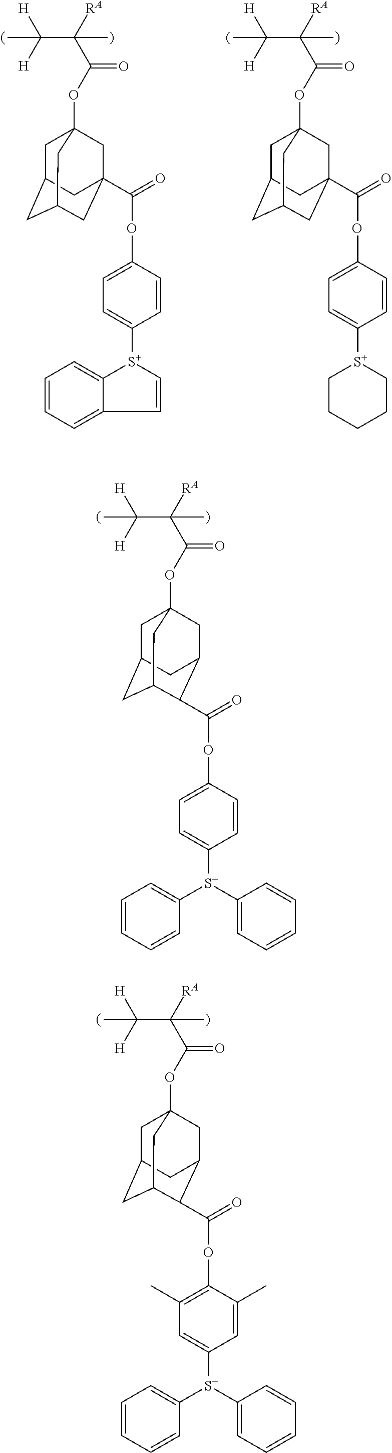

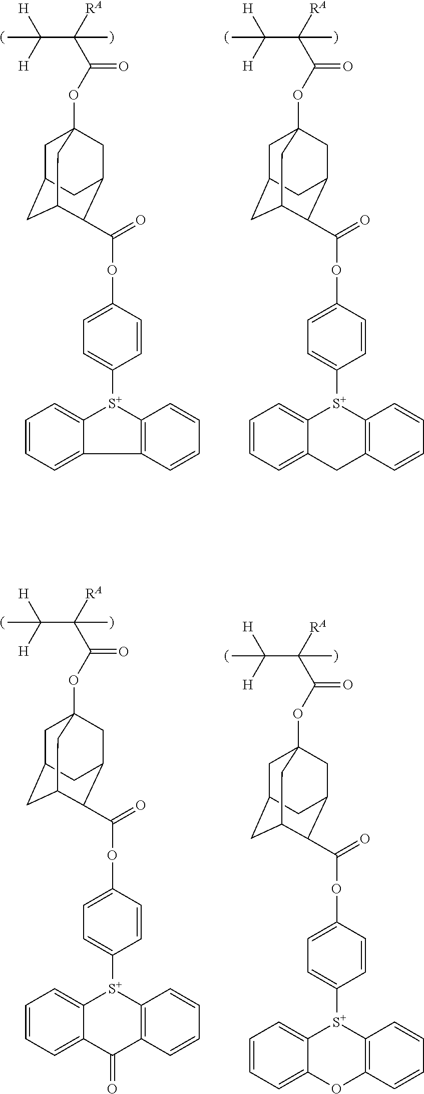

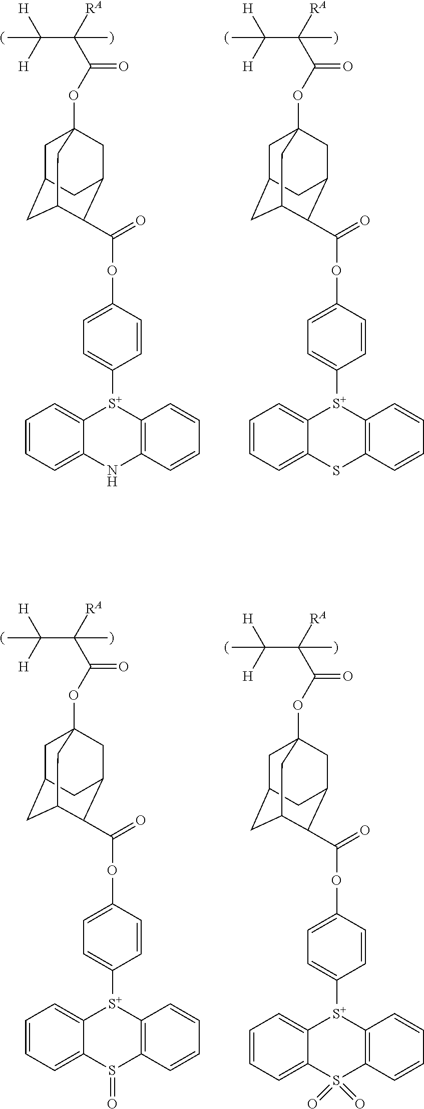

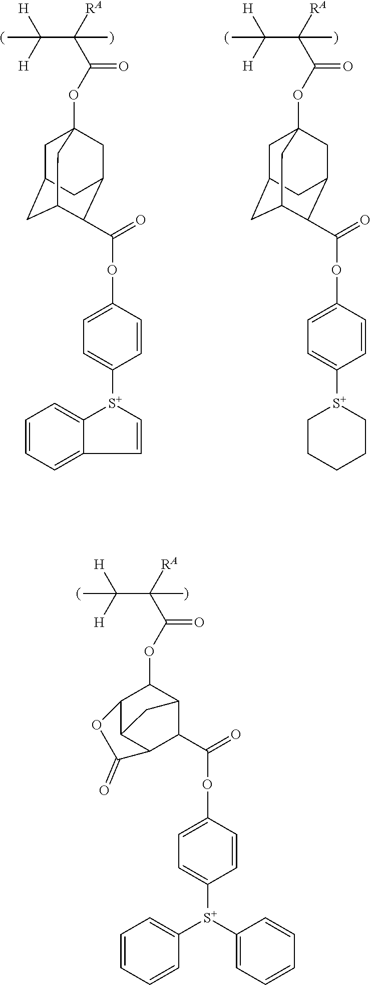

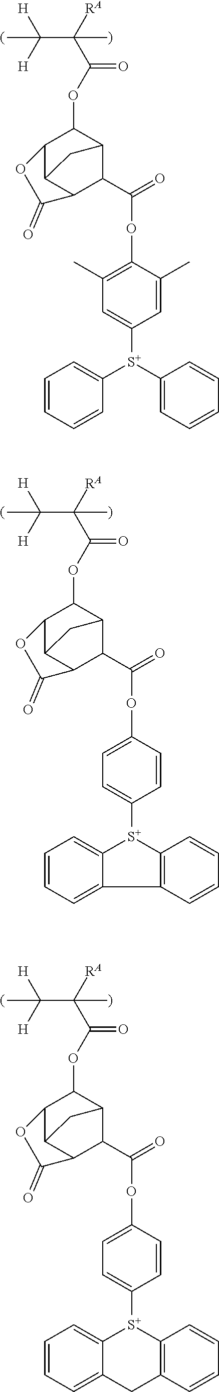

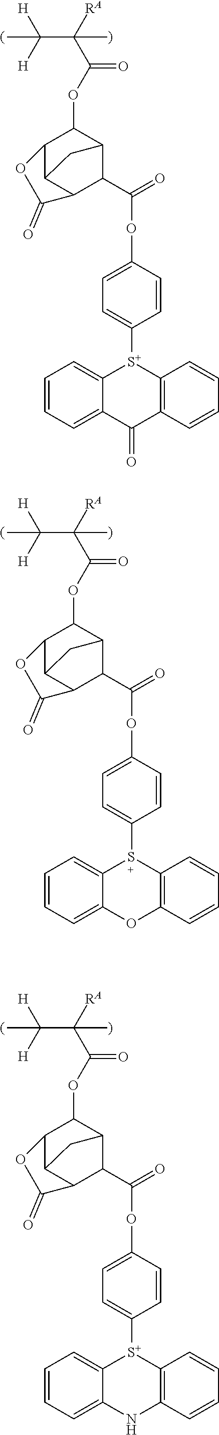

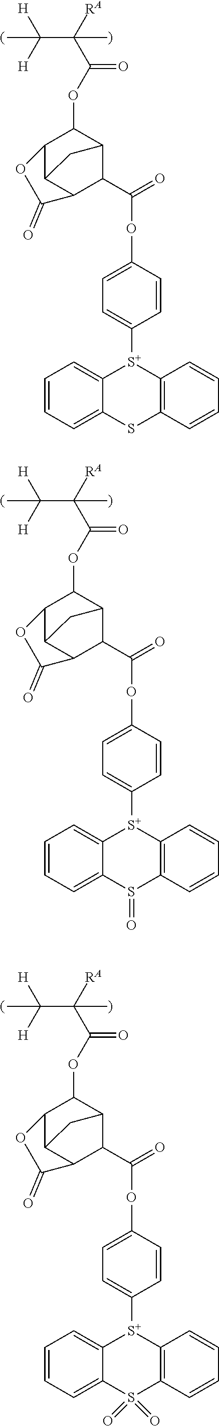

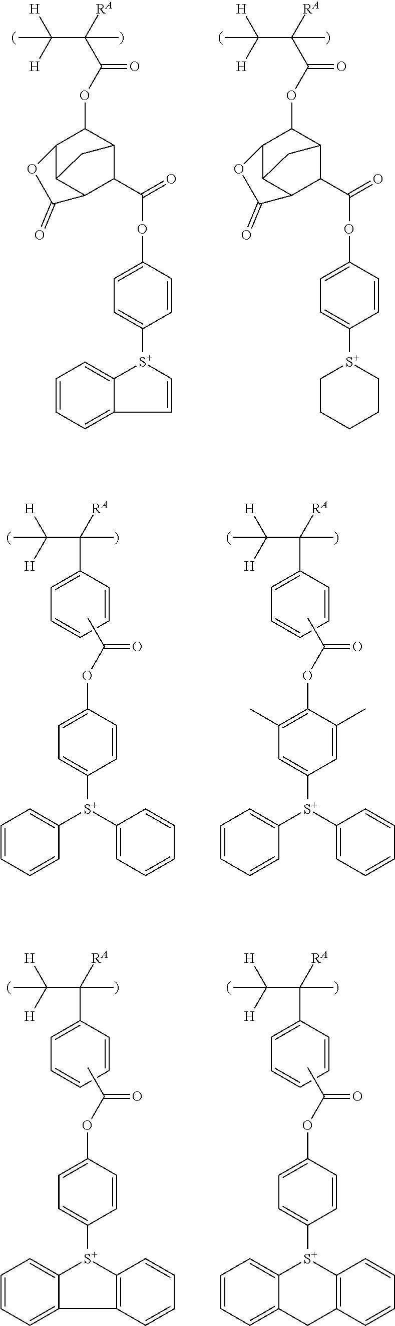

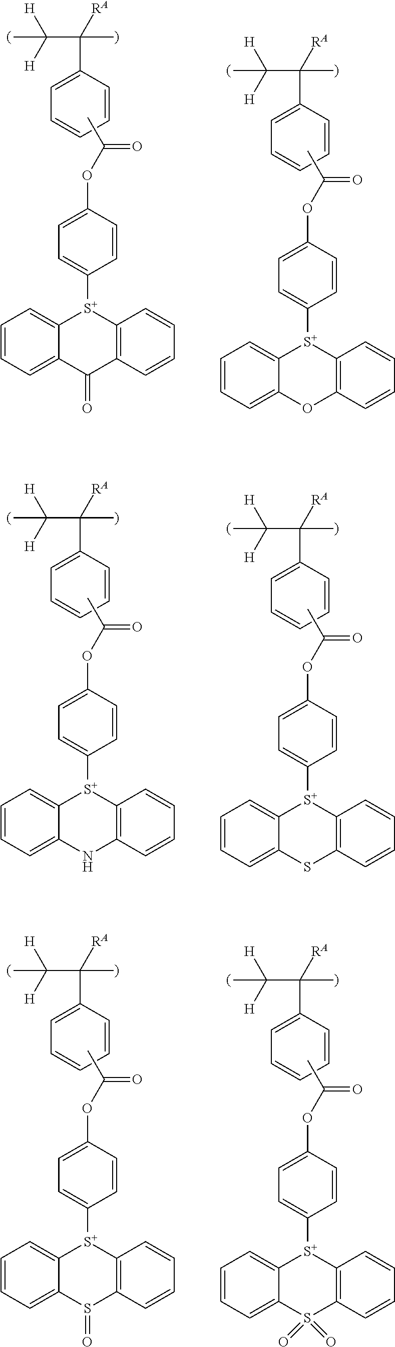

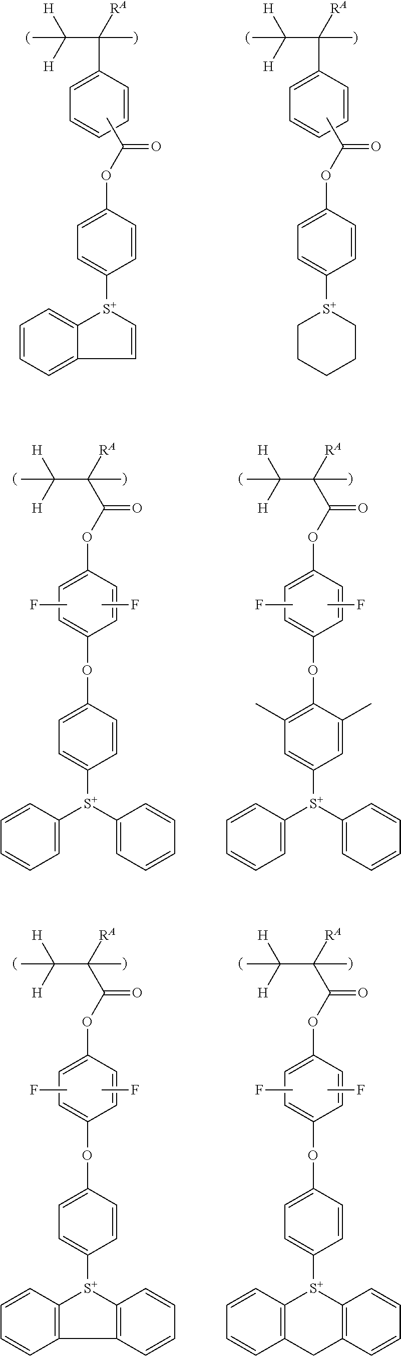

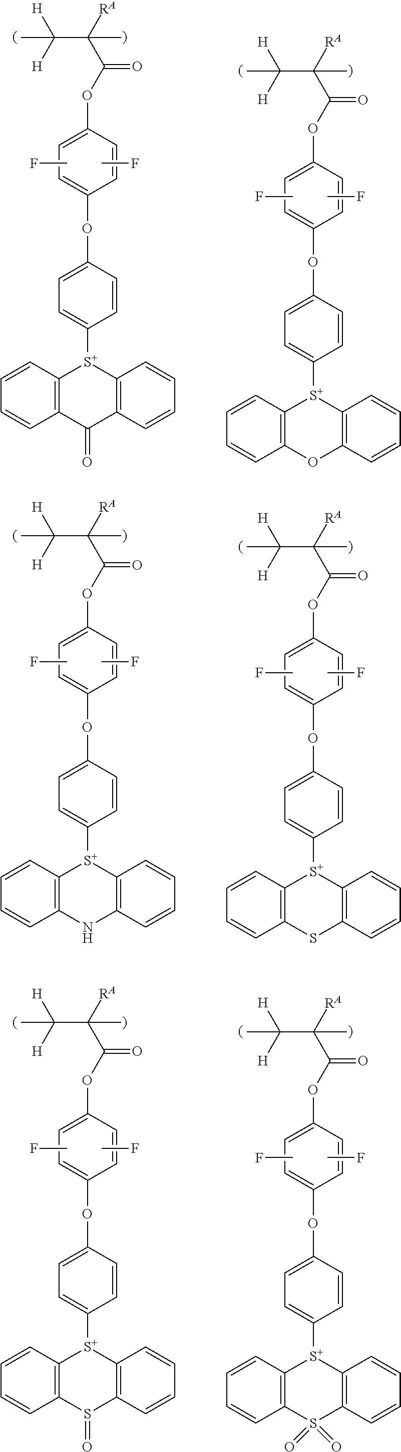

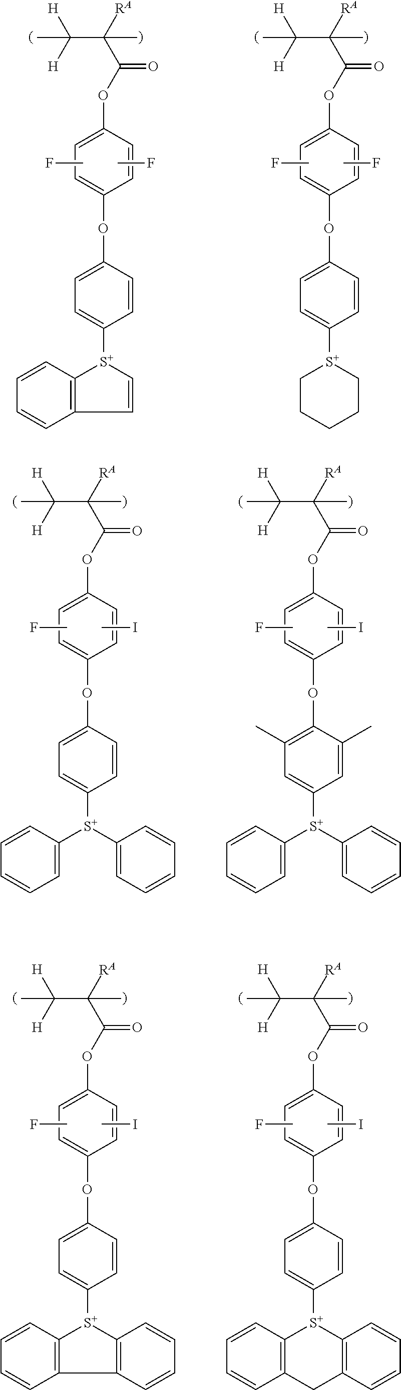

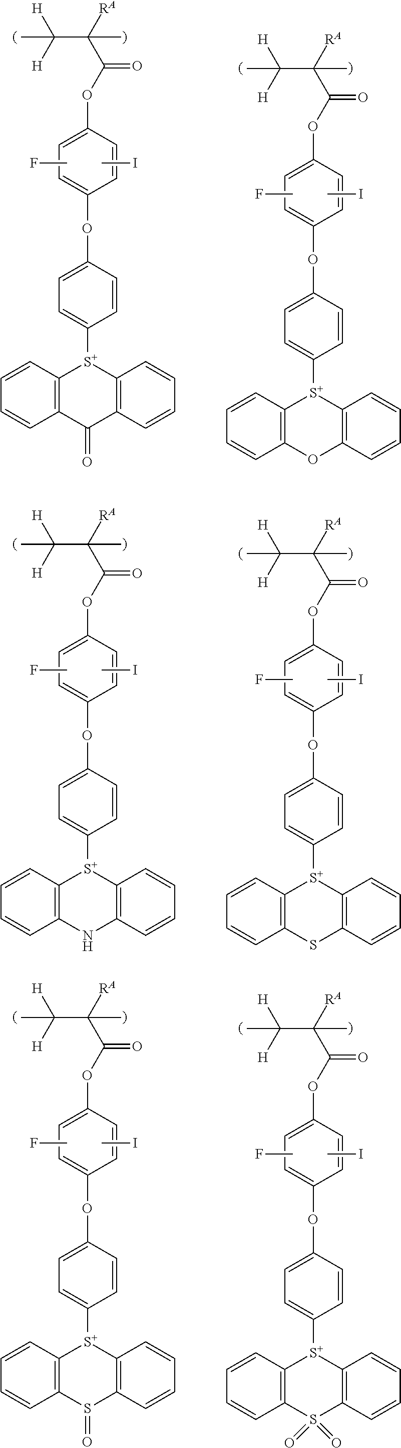

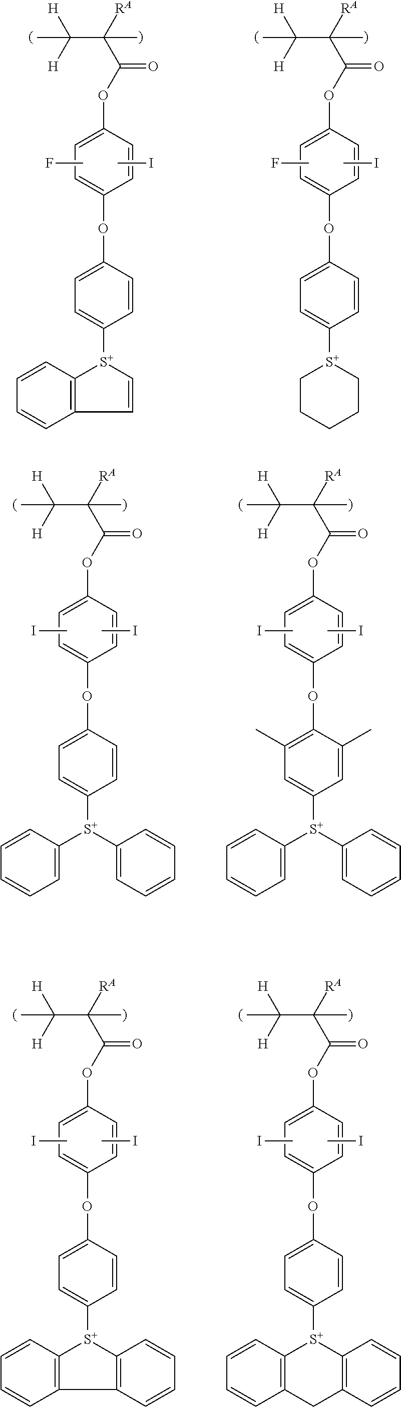

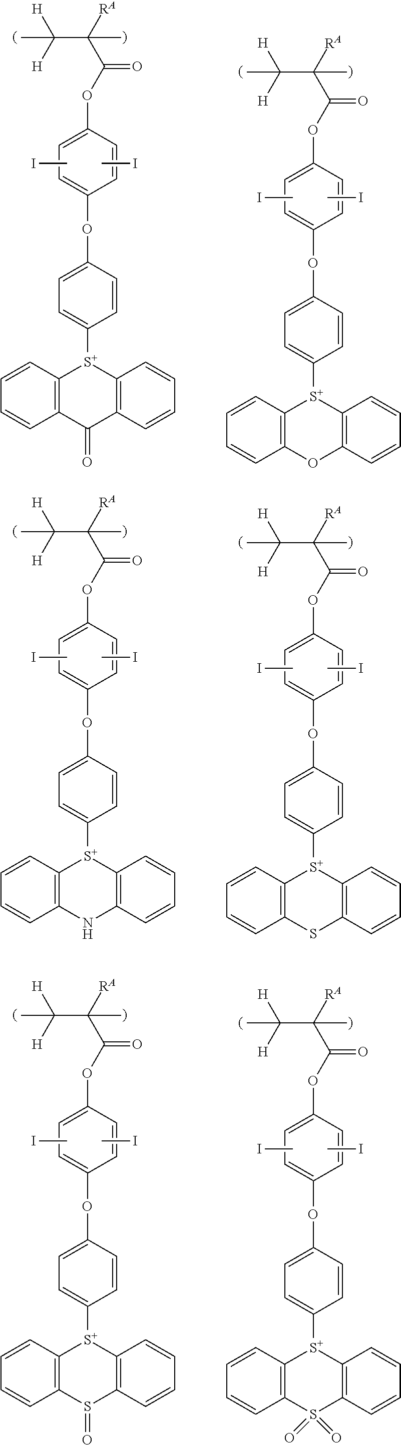

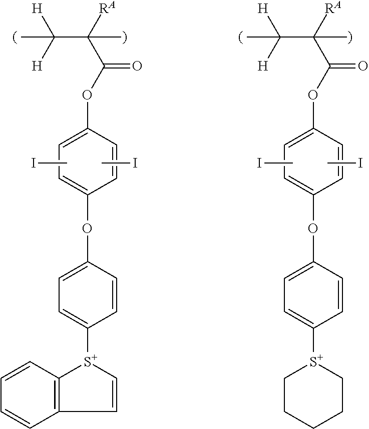

In formula (c1), M⁻ is a non-nucleophilic counter ion. The non-nucleophilic counter ion is preferably a halide ion, a sulfonate anion, an imide anion, or a methide anion. Specific examples of the halide ion include a chloride ion and a bromide ion. Specific examples of the sulfonate anion (sulfonate ion) include fluoroalkylsulfonate ions such as a triflate ion, a 1,1,1-trifluoroethanesulfonate ion, and a nonafluorobutanesulfonate ion; arylsulfonate ions such as a tosylate ion, a benzenesulfonate ion, a 4-fluorobenzenesulfonate ion, and a 1,2,3,4,5-pentafluorobenzenesulfonate ion; alkylsulfonate ions such as a mesylate ion and a butanesulfonate ion. Specific examples of the imide anion (imide ion) include a bis(trifluoromethylsulfonyl)imide ion, a bis(perfluoroethylsulfonyl)imide, and a bis(perfluorobutylsulfonyl)imide ion. Specific examples of the methide anion (methide ion) include a tris(trifluoromethylsulfonyl)methide ion and a tris(perfluoroethylsulfonyl)methide ion.

Other examples of the non-nucleophilic counter ion include anions having the formulae (c1-1) to (c1-4).

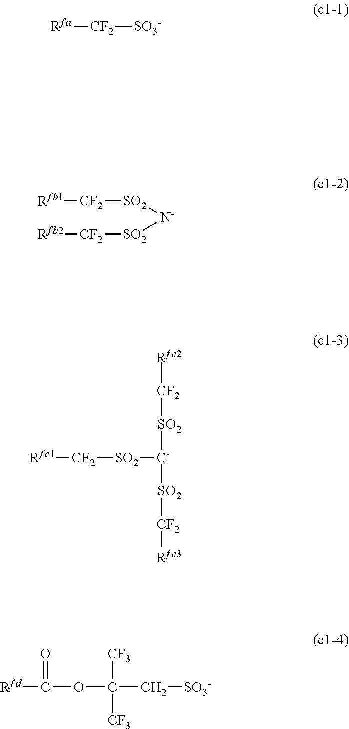

In formula (c1-1), R^{fa} is a fluorine atom or a C₁-C₄₀ hydrocarbyl group which may contain a heteroatom. The hydrocarbyl group may be saturated or unsaturated and straight, branched, or cyclic. Specific examples thereof are as exemplified below for the hydrocarbyl group R^{fa1} in formula (c1-1-1).

The anion having formula (c1-1) is preferably an anion having the formula (c1-1-1).

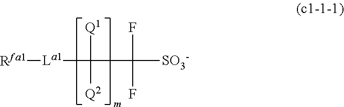

In formula (c1-1-1), Q¹ and Q² are each independently a hydrogen atom, a fluorine atom, or a C₁-C₆ fluorinated saturated hydrocarbyl group, and at least one of Q¹ and Q² is preferably a trifluoromethyl group for improving solvent solubility. m is 0, 1, 2, 3, or 4, and is particularly preferably 1. R^{fa1} is a C₁-C₃₅ hydrocarbyl group which may contain a heteroatom. The heteroatom is preferably an oxygen atom, a nitrogen atom, a sulfur atom, a halogen atom, or the like, and more preferably an oxygen atom. As the hydrocarbyl group, a C₆-C₃₀ hydrocarbyl group is particularly preferred from the viewpoint of obtaining high resolution in small-size pattern formation.

In formula (c1-1-1), the C₁-C₃₅ hydrocarbyl group R^{fa1} may be saturated or unsaturated and straight, branched, or cyclic. Specific examples thereof include C₁-C₃₅ alkyl groups such as a methyl group, an ethyl group, an n-propyl group, an isopropyl group, an n-butyl group, an isobutyl group, a sec-butyl group, a tert-butyl group, a pentyl group, a neopentyl group, a hexyl group, a heptyl group, a 2-ethylhexyl group, a nonyl group, an undecyl group, a tridecyl group, a pentadecyl group, a heptadecyl group, and an icosyl group; C₃-C₃₅ cyclic saturated hydrocarbyl groups such as a cyclopentyl group, a cyclohexyl group, a 1-adamantyl group, a 2-adamantyl group, a 1-adamantylmethyl group, a norbornyl group, a norbornylmethyl group, a tricyclodecyl group, a tetracyclododecyl group, a tetracyclododecylmethyl group, and a dicyclohexylmethyl group; C₂-C₃₅ unsaturated aliphatic hydrocarbyl groups such as a 2-propenyl group and a 3-cyclohexenyl group; C₆-C₃₅ aryl groups such as a phenyl group, a 1-naphthyl group, a 2-naphthyl group, and a 9-fluorenyl group; C₇-C₃₅ aralkyl groups such as a benzyl group and a diphenylmethyl group; and combinations thereof.

In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a nitro group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. Specific examples of the hydrocarbyl group containing a heteroatom include a tetrahydrofuryl group, a methoxymethyl group, an ethoxymethyl group, a methylthiomethyl group, an acetamidomethyl group, a trifluoroethyl group, a (2-methoxyethoxy)methyl group, an acetoxymethyl group, a 2-carboxy-1-cyclohexyl group, a 2-oxopropyl group, a 4-oxo-1-adamantyl group, and a 3-oxocyclohexyl group.

In formula (c1-1-1), L^{a1} is a single bond, an ether bond, an ester bond, a sulfonate ester bond, a carbonate bond, or a carbamate bond, and from the viewpoint of synthesis, an ether bond or an ester bond is preferred, and an ester bond is more preferred.

Specific examples of the anion of having formula (c1-1) are shown below, but are not limited thereto. Herein Q¹ is as defined above, and Ac is an acetyl group.

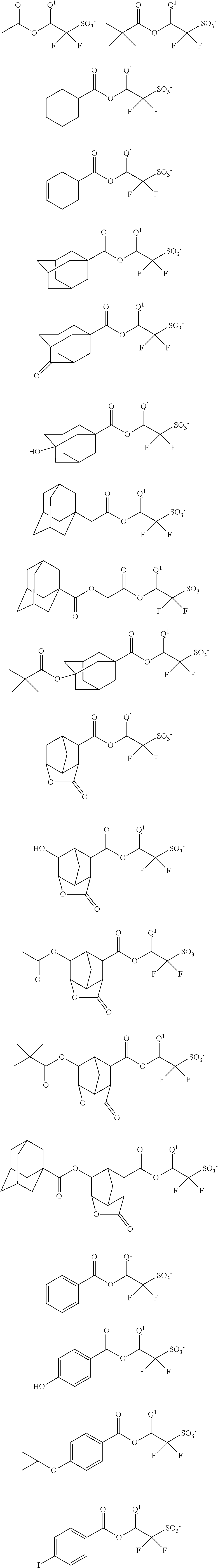

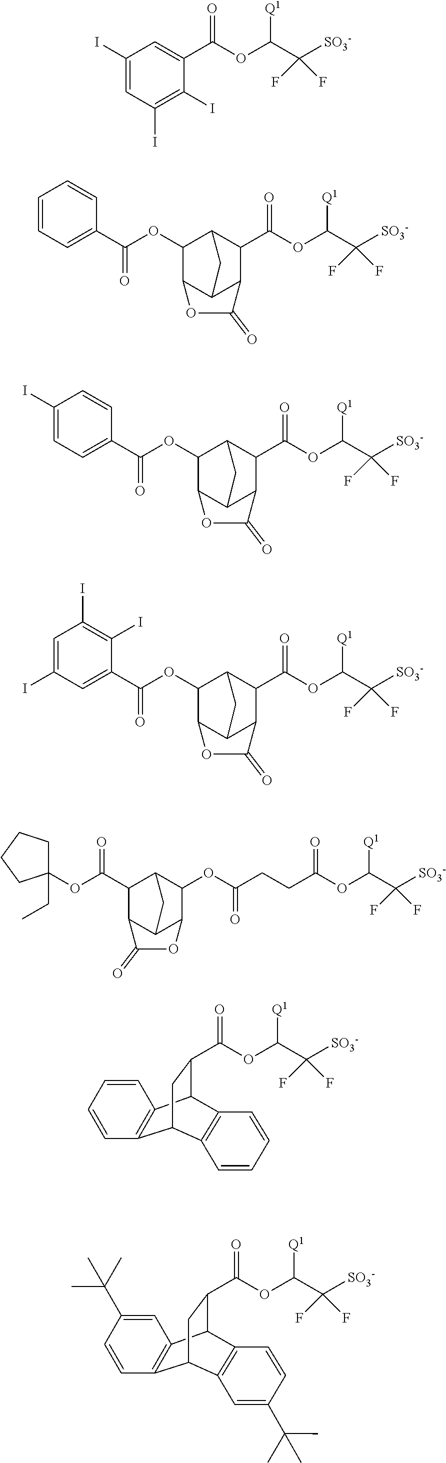

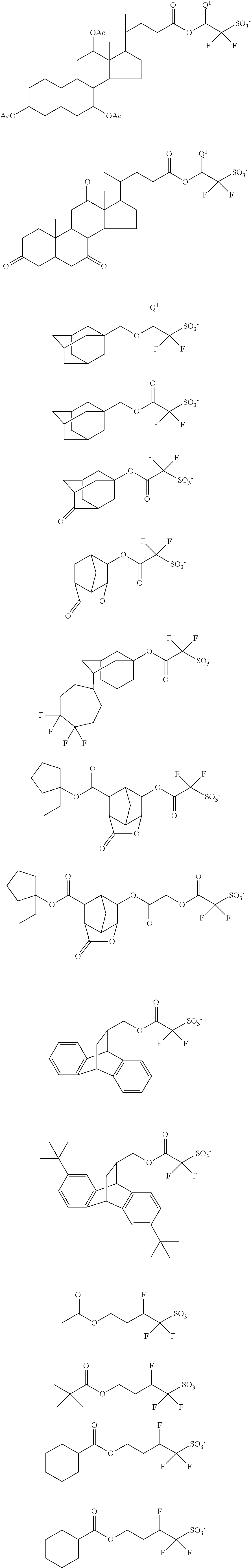

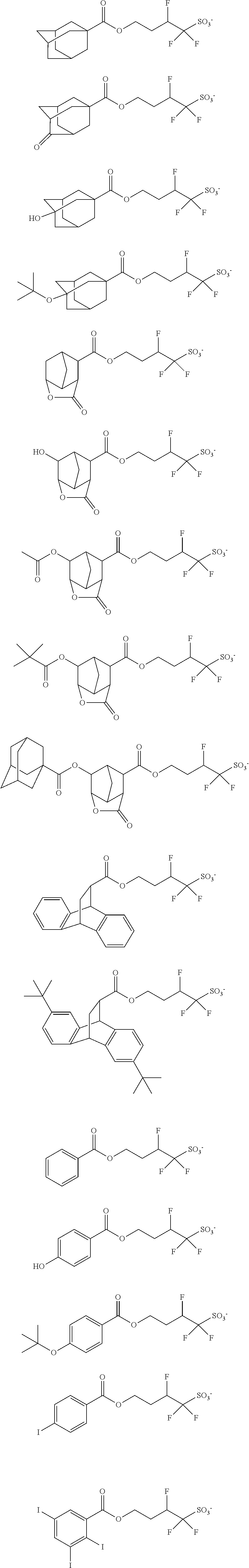

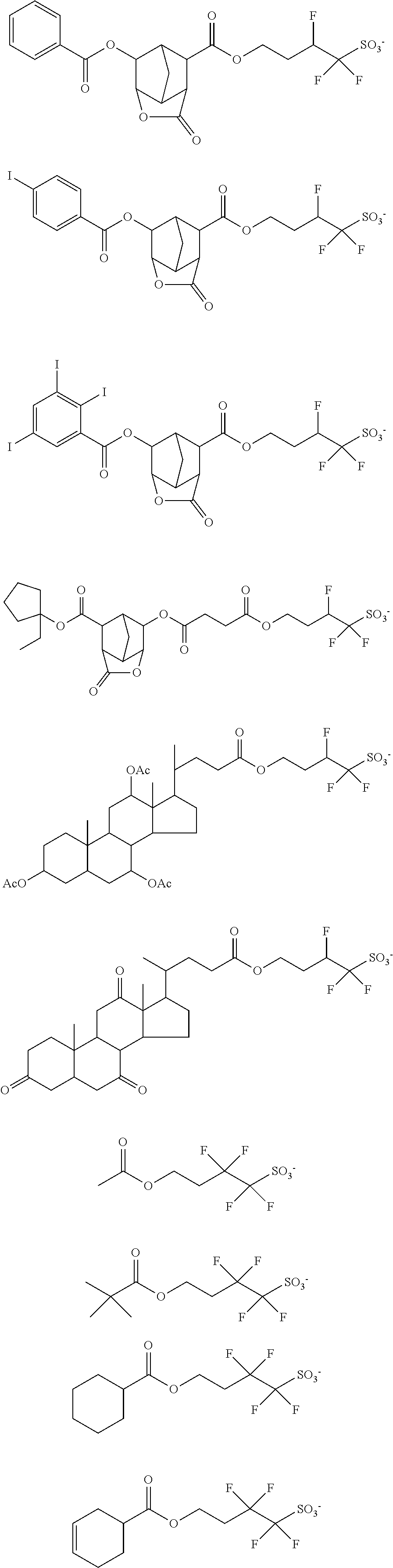

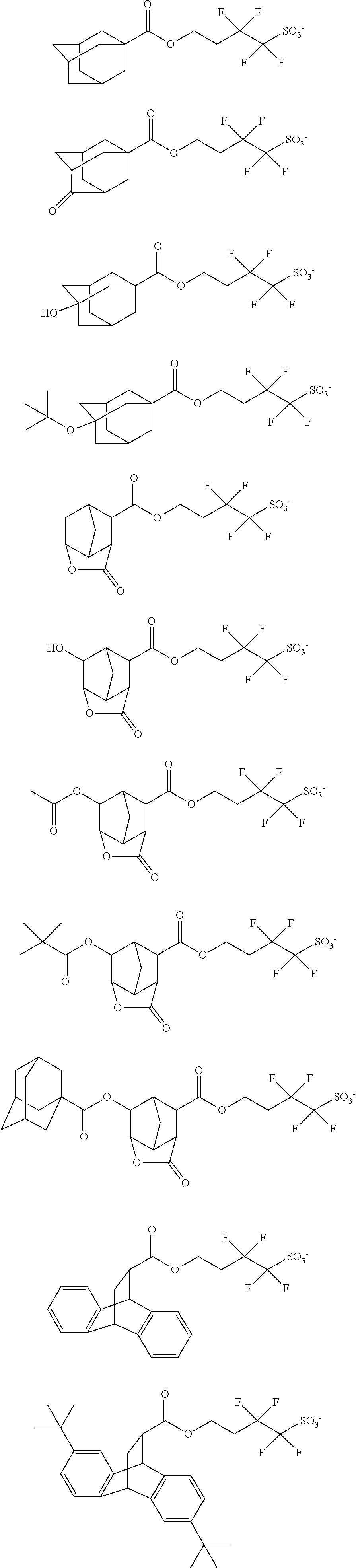

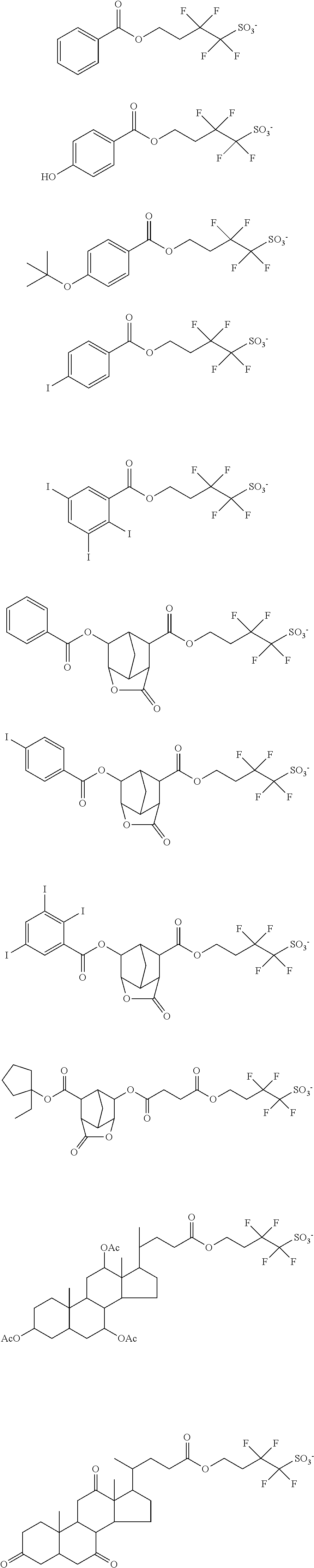

In formula (c1-2), R^{fb1} and R^{fb2} are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified for the hydrocarbyl group R^{fa1} in formula (c1-1-1). R^{fb1} and R^{fb2} are preferably a fluorine atom or a C₁-C₄ straight fluorinated alkyl group. R^{fb1} and R^{fb2} may bond together to form a ring with the group (—CF₂—SO₂—N⁻—SO₂—CF₂—) to which they are attached. In this case, the group obtained by bonding R^{fb1} and R^{fb2} together is preferably a fluorinated ethylene group or a fluorinated propylene group.

In formula (c1-3), R^{fc1}, R^{fc2}, and R^{fc3} are each independently a fluorine atom or a C₁-C₄₀ hydrocarbyl group which may contain a heteroatom. The hydrocarbyl group may be saturated or unsaturated and straight, branched, or cyclic. Specific examples thereof are as exemplified for the hydrocarbyl group R^{fa1} in formula (c1-1-1). R^{fc1}, R^{fc2}, and R^{fc3} are preferably a fluorine atom or a C₁-C₄ straight fluorinated alkyl group. R^{fc1} and R^{fc2} may bond together to form a ring with the group (—CF₂—SO₂—C⁻—SO₂—CF₂—) to which they are attached. In this case, the group obtained by bonding R^{fc1} and R^{fc2} together is preferably a fluorinated ethylene group or a fluorinated propylene group.

In formula (c1-4), R^{fd} is a C₁-C₄₀ hydrocarbyl group which may contain a heteroatom. The hydrocarbyl group may be saturated or unsaturated and straight, branched, or cyclic. Specific examples thereof are as exemplified for the hydrocarbyl group R^{fa1} in formula (c1-1-1).

Specific examples of the anion of having formula (c1-4) are shown below, but are not limited thereto.

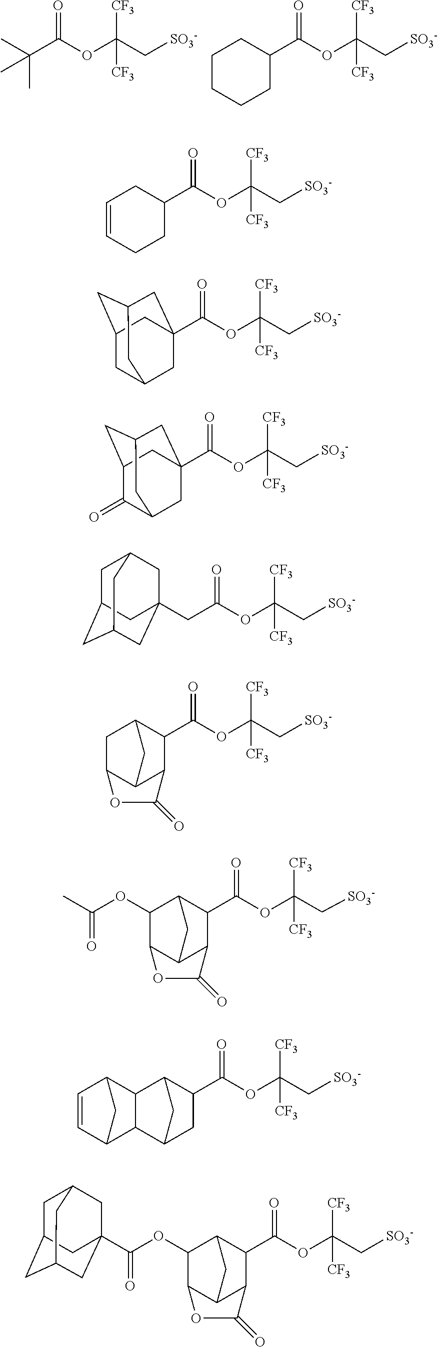

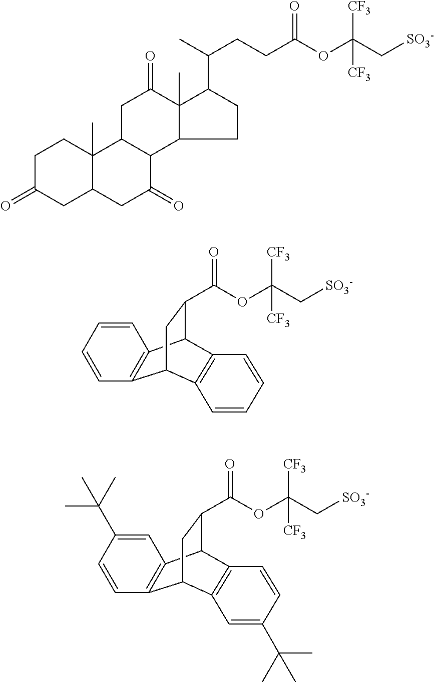

Examples of the non-nucleophilic counter ion further include an anion having an aromatic ring substituted by an iodine atom or a bromine atom. Specific examples of the anion include those having the formula (c1-5).

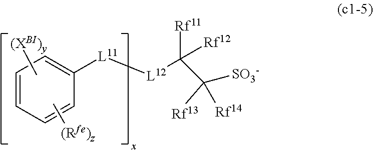

In formula (c1-5), x is 1, 2, or 3, y is 1, 2, 3, 4, or 5, z is 0, 1, 2, or 3. Provided that 1≤y+z≤5. y is preferably 1, 2, or 3, and more preferably 2 or 3. z is preferably 0, 1, or 2.

In formula (c1-5), X^{BI} is an iodine atom or a bromine atom, and a plurality of X^{BI} may be identical or different when x and/or y is 2 or more.

In formula (c1-5), L11 is a single bond, an ether bond, an ester bond, or a C₁-C₆ saturated hydrocarbylene group which may contain an ether bond or ester bond. The saturated hydrocarbylene group may be straight, branched, or cyclic.

In formula (c1-5), L12 is a single bond or a C₁-C₂₀ divalent linking group when x=1, or a C₁-C₂₀ (x+1)-valent linking group when x=2 or 3. The linking group may contain an oxygen atom, a sulfur atom, or a nitrogen atom.

In formula (c1-5), R^{fe} is a hydroxy group, a carboxy group, a fluorine atom, a chlorine atom, a bromine atom, an amino group, or a C₁-C₂₀ hydrocarbyl group, a C₁-C₂₀ hydrocarbyloxy group, a C₂-C₂₀ hydrocarbylcarbonyl group, a C₂-C₂₀ hydrocarbyloxycarbonyl group, a C₂-C₂₀ hydrocarbylcarbonyloxy group, or a C₁-C₂₀ hydrocarbylsulfonyloxy group, which may contain a fluorine atom, a chlorine atom, a bromine atom, a hydroxy group, an amino group, or an ether bond, or —N(R^{feA})(R^{feB}), —N(R^{feC})—C(═O)—R^{feD}, or —N(R^{feC})—C(═O)—O—R^{feD}. R^{feA} and R^{feB} are each independently a hydrogen atom or a C₁-C₆ saturated hydrocarbyl group. R^{feC} is a hydrogen atom, or a C₁-C₆ saturated hydrocarbyl group which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. R^{feD} is a C₁-C₁₆ aliphatic hydrocarbyl group, a C₆-C₁₂ aryl group, or a C₇-Cis aralkyl group, which may contain a halogen atom, a hydroxy group, a C₁-C₆ saturated hydrocarbyloxy group, a C₂-C₆ saturated hydrocarbylcarbonyl group, or a C₂-C₆ saturated hydrocarbylcarbonyloxy group. The aliphatic hydrocarbyl group may be saturated or unsaturated and straight, branched, or cyclic. The hydrocarbyl group, the hydrocarbyloxy group, the hydrocarbylcarbonyl group, the hydrocarbyloxycarbonyl group, the hydrocarbylcarbonyloxy group, and the hydrocarbylsulfonyloxy group may be straight, branched, or cyclic. A plurality of R^{fe} may be identical or different when x and/or z is 2 or more. Among them, R^{fe} is preferably a hydroxy group, —N(R^{feC})—C(═O)—R^{feD}, —N(R^{feC})—C(═O)—O—R^{feD}, a fluorine atom, a chlorine atom, a bromine atom, a methyl group, or a methoxy group.

In formula (c1-5), Rf¹¹ to Rf¹⁴ are each independently a hydrogen atom, a fluorine atom, or a trifluoromethyl group, and at least one of Rf¹¹ to Rf¹⁴ is a fluorine atom or a trifluoromethyl group. Rf¹¹ and Rf¹², taken together, may form a carbonyl group. In particular, both Rf¹³ and Rf¹⁴ are preferably fluorine atoms.

Specific examples of the anion of having formula (c1-5) are shown below, but are not limited thereto. Herein X^{BI} is as defined above.

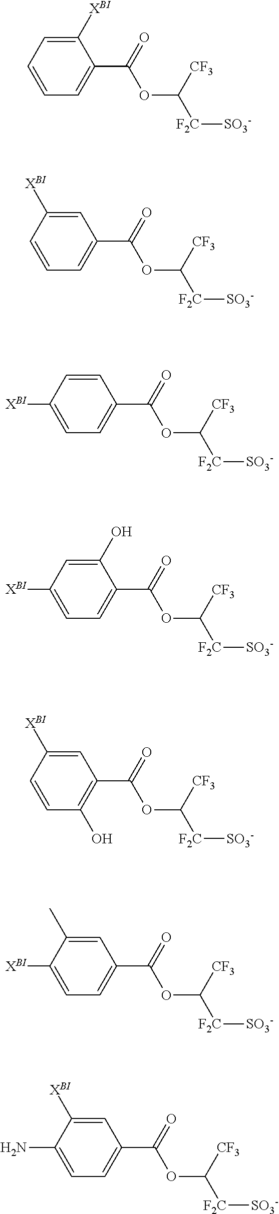

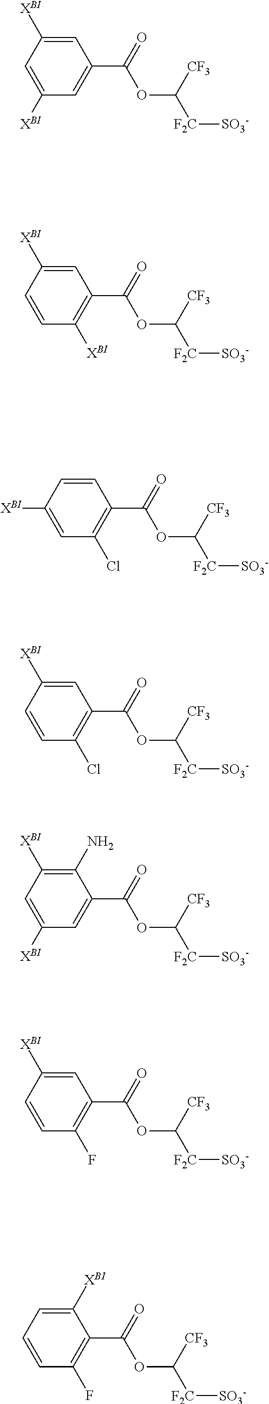

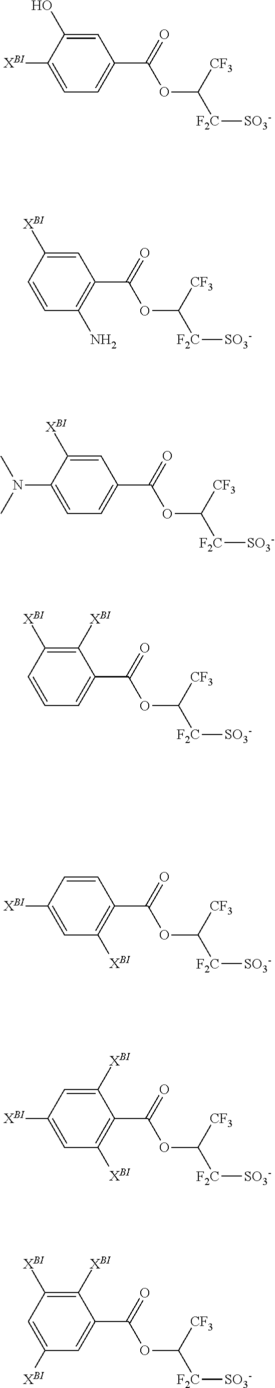

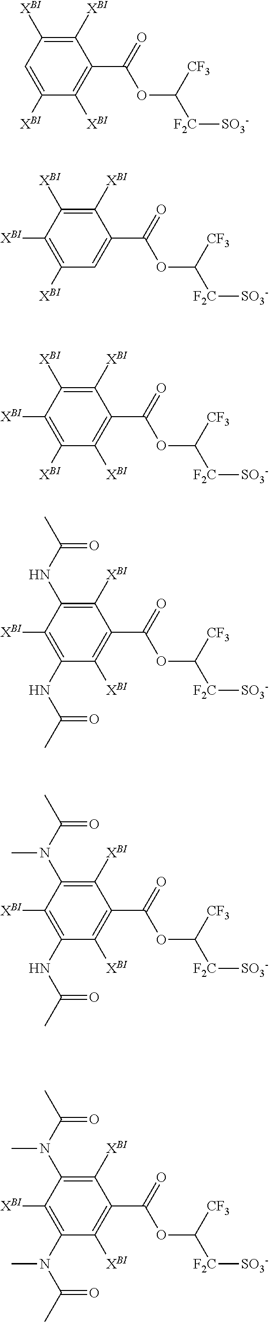

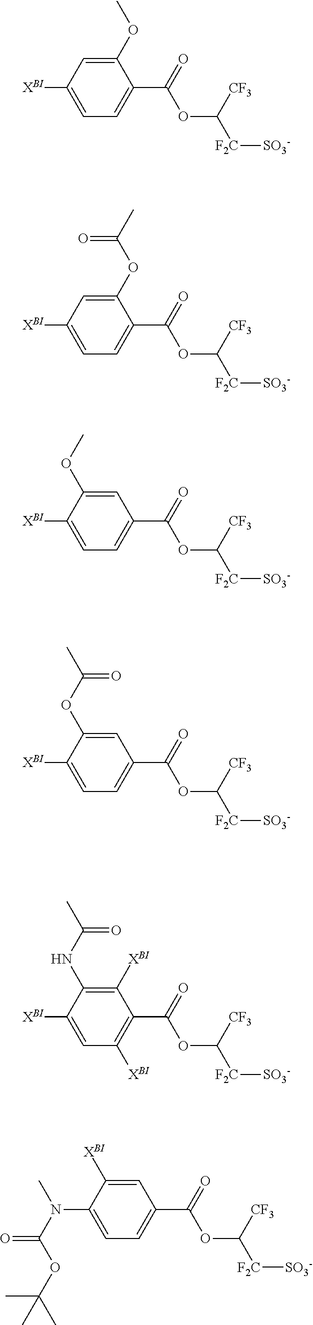

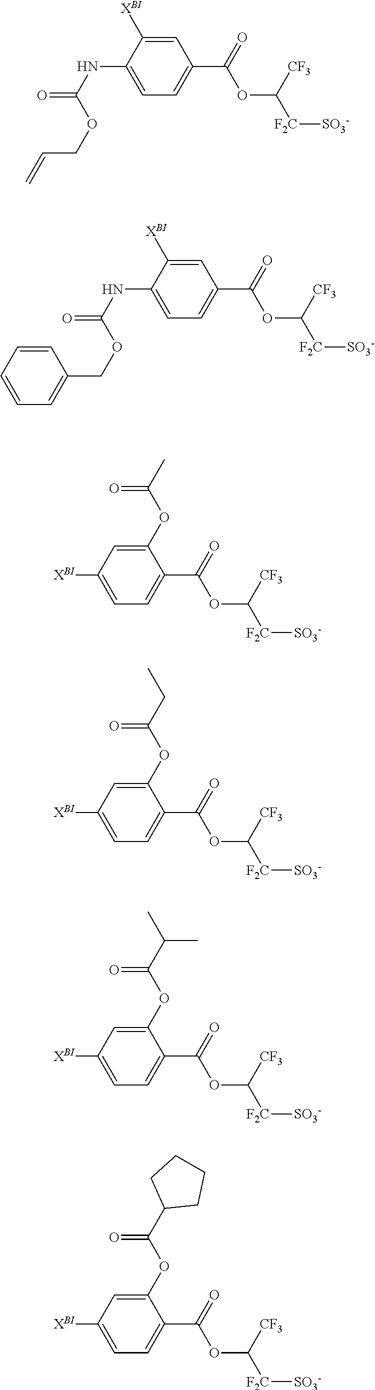

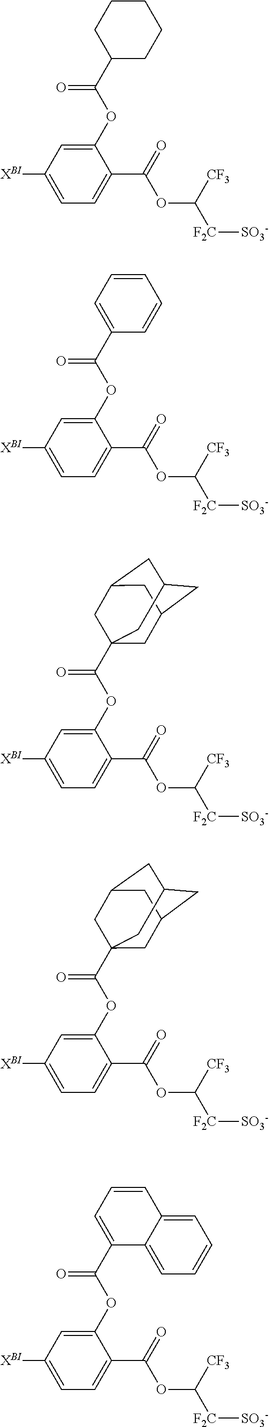

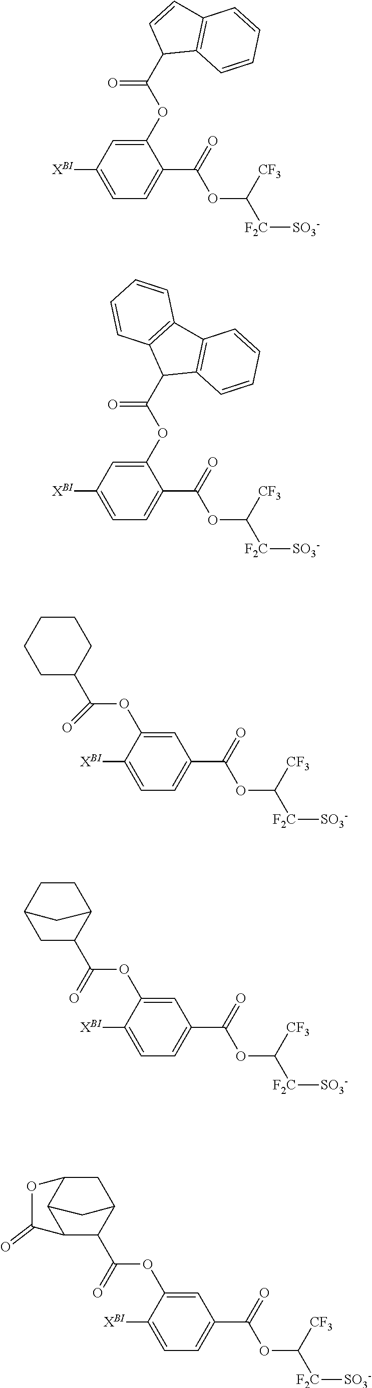

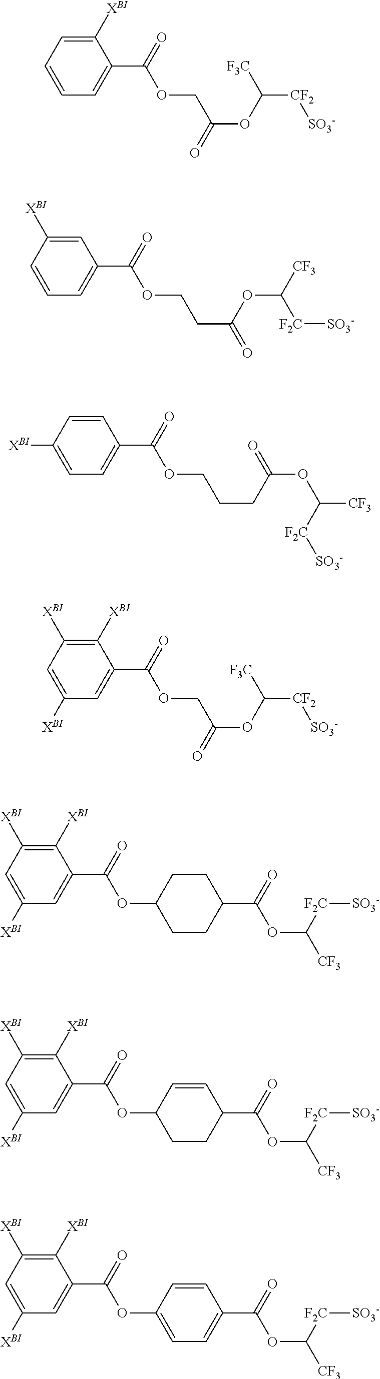

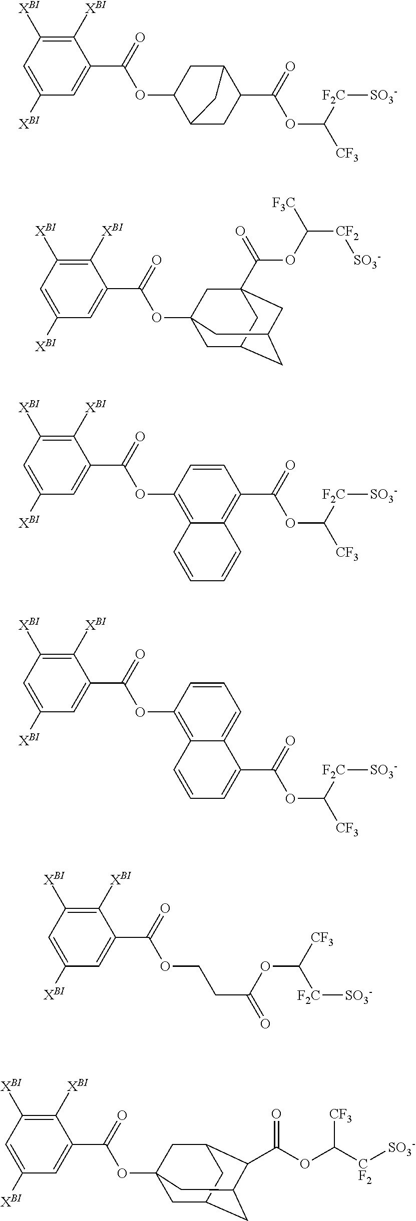

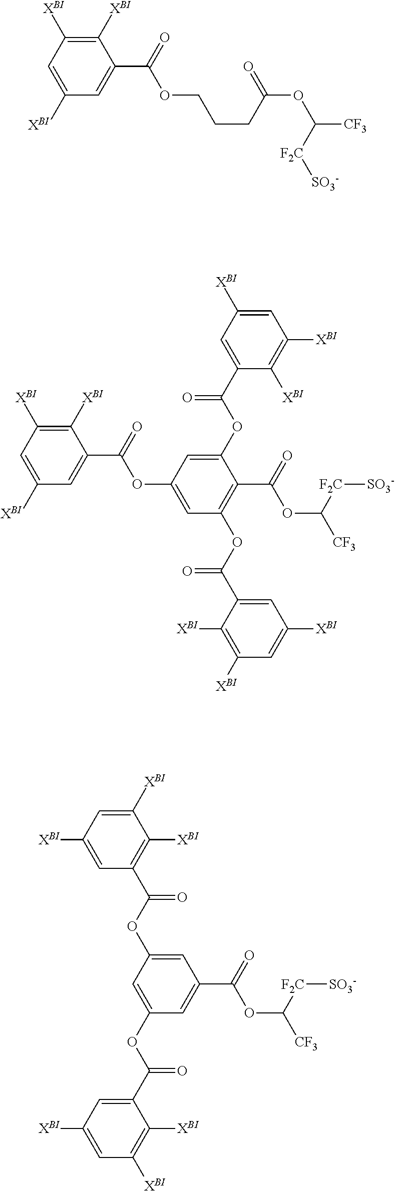

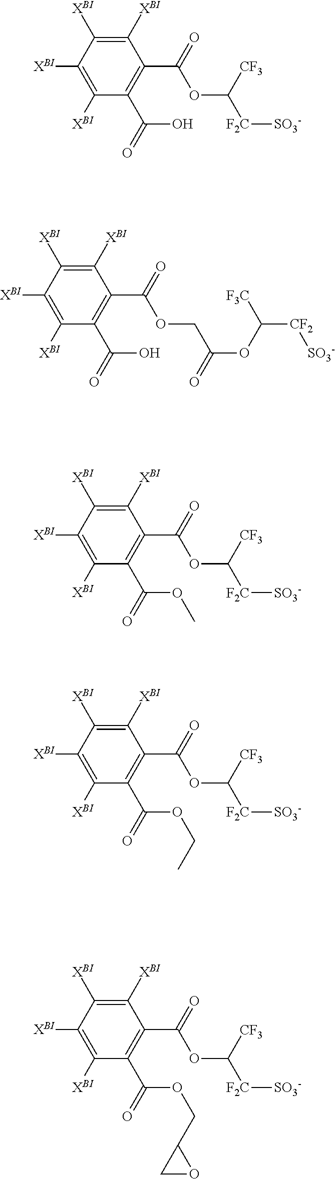

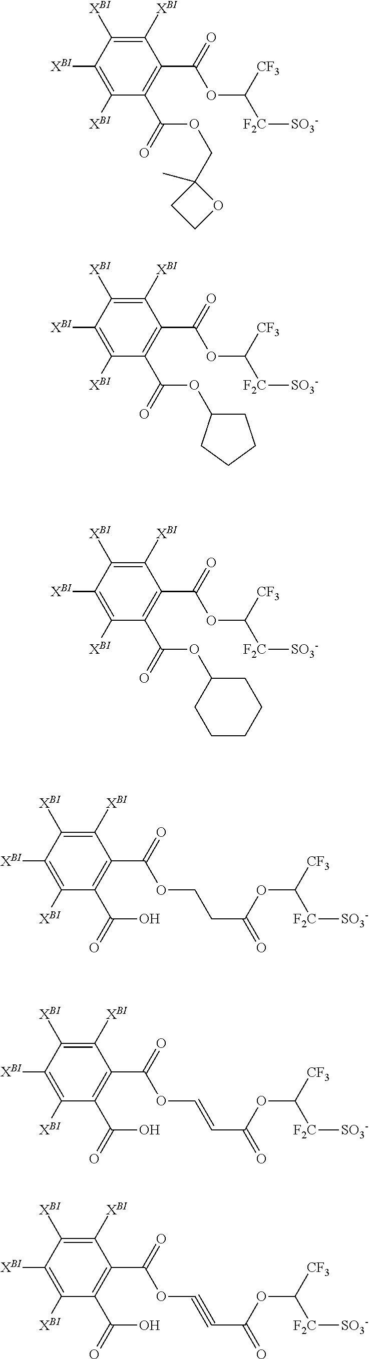

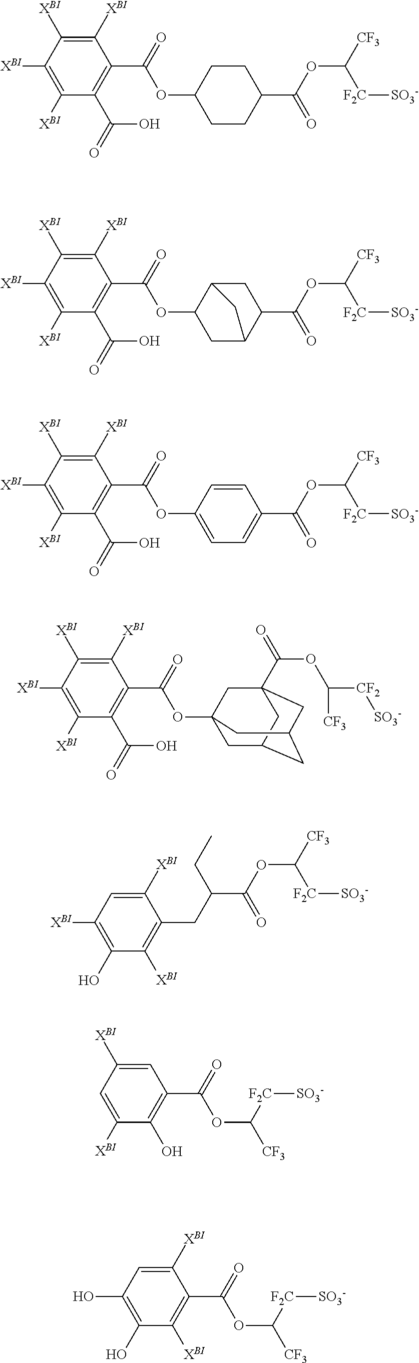

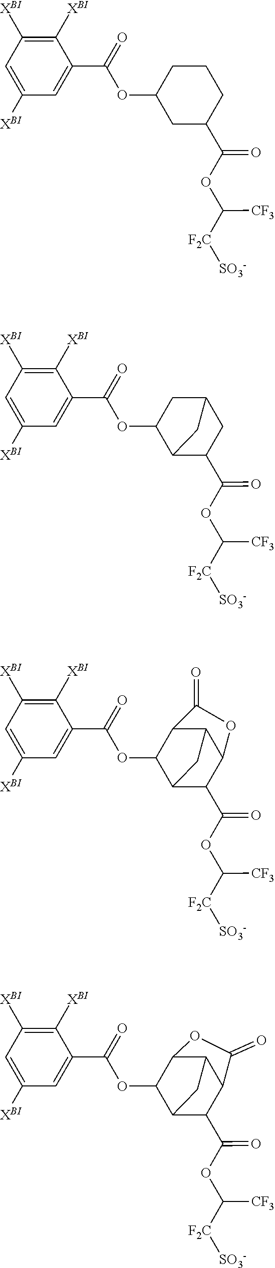

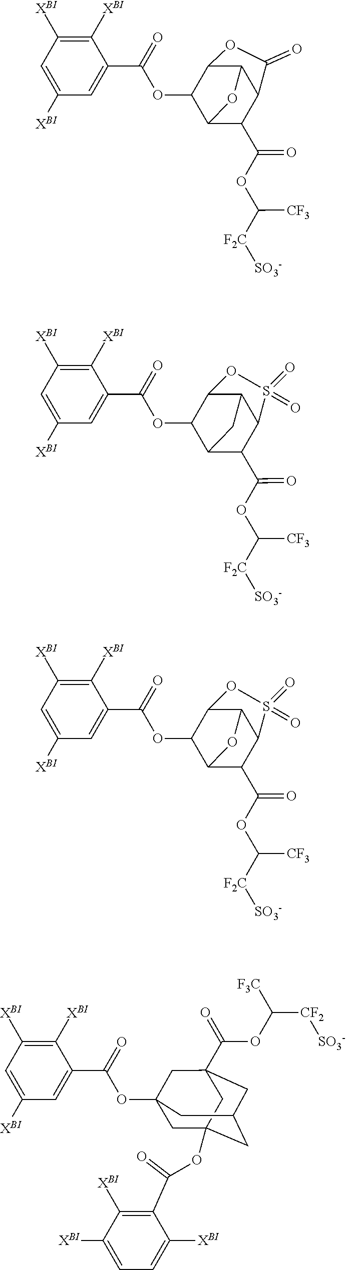

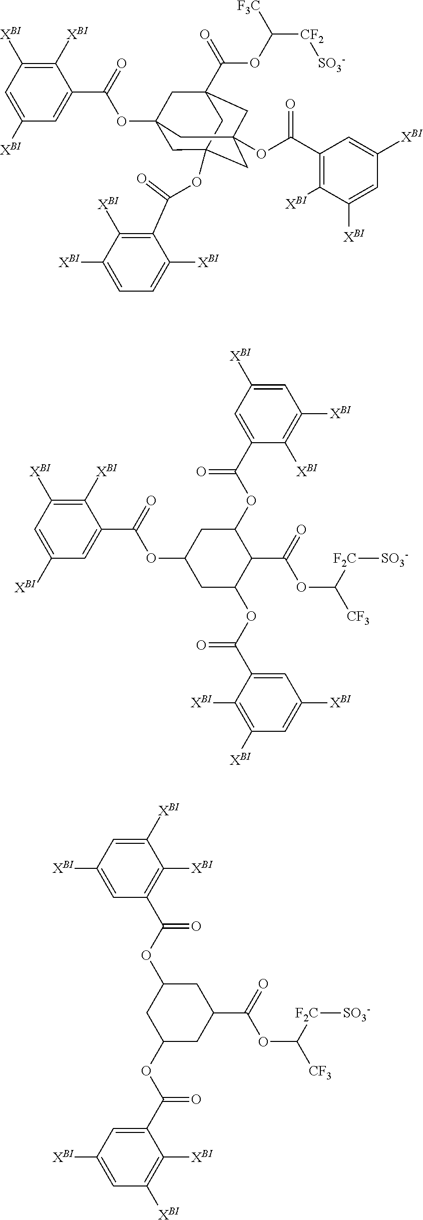

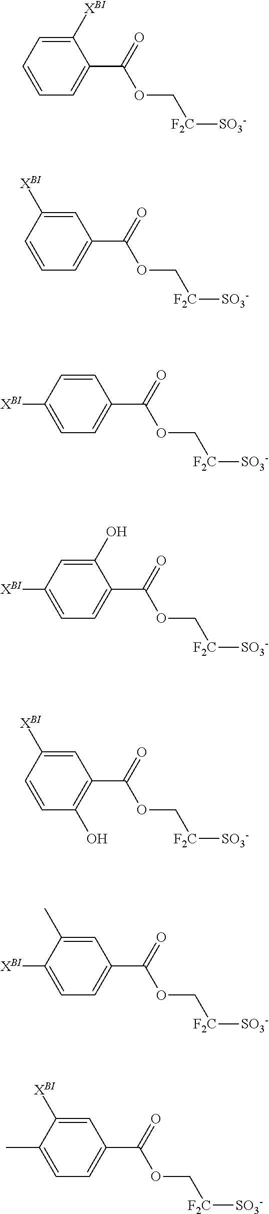

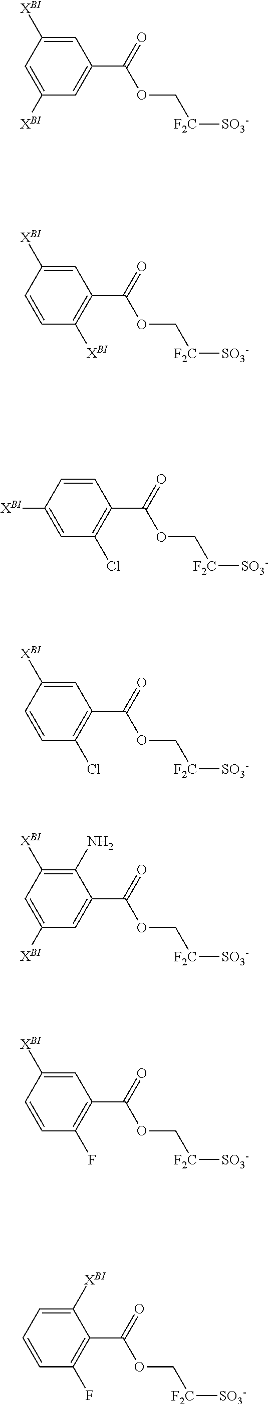

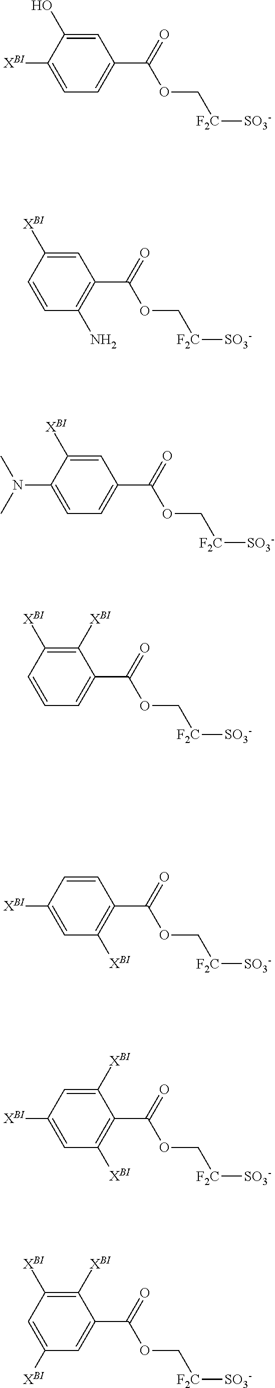

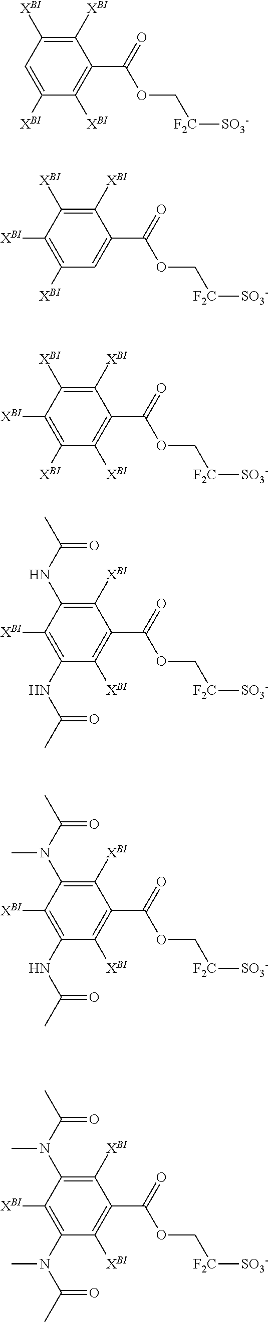

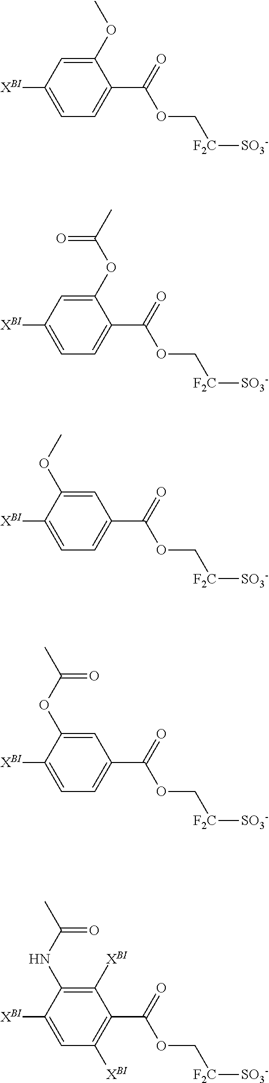

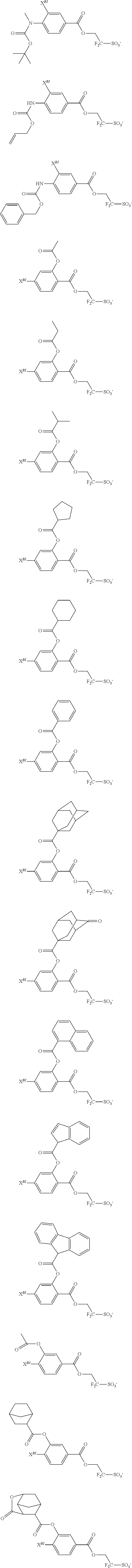

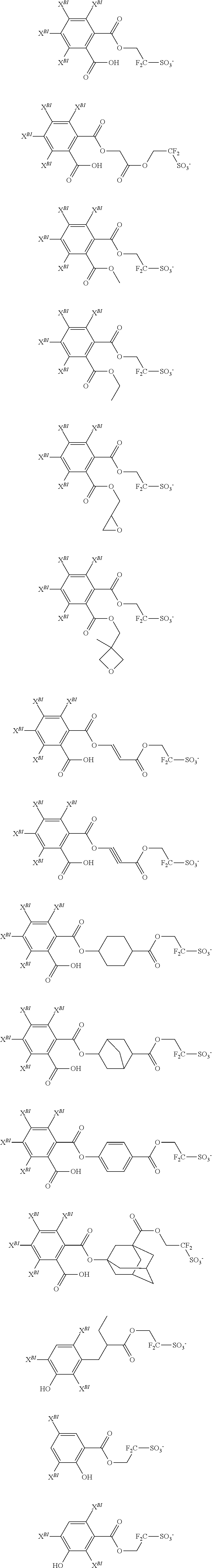

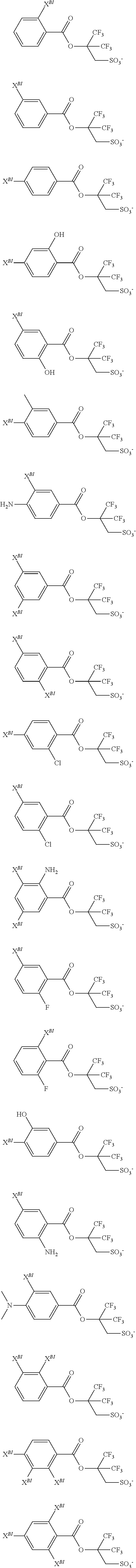

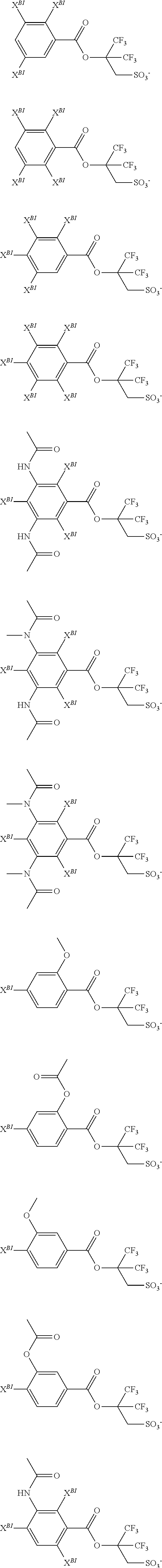

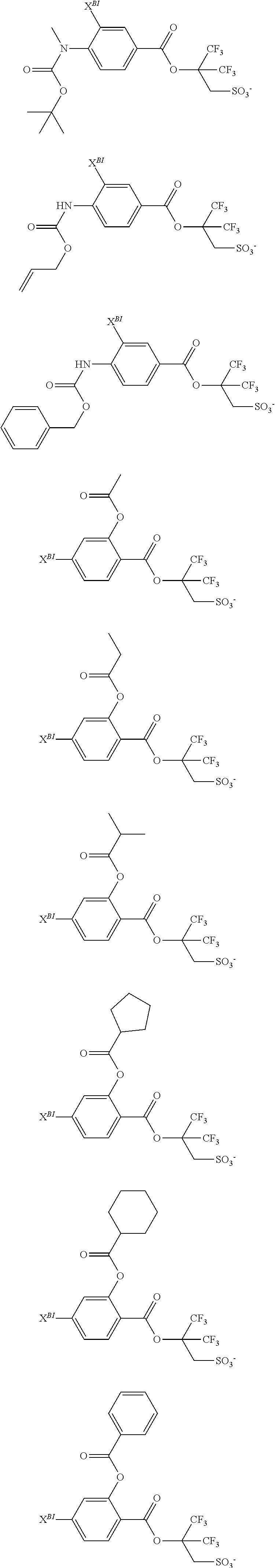

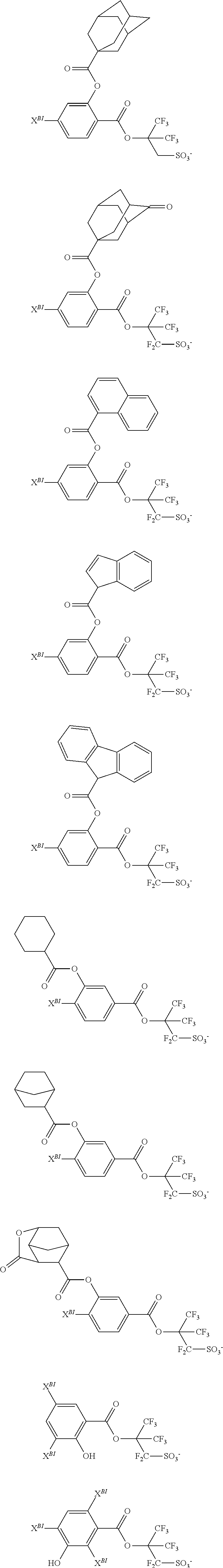

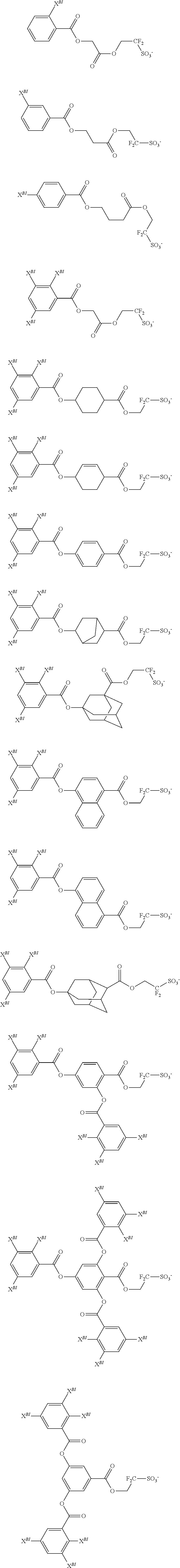

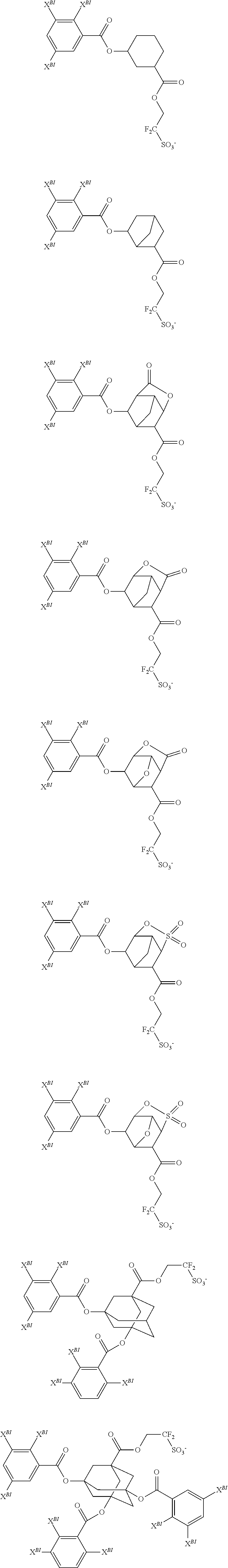

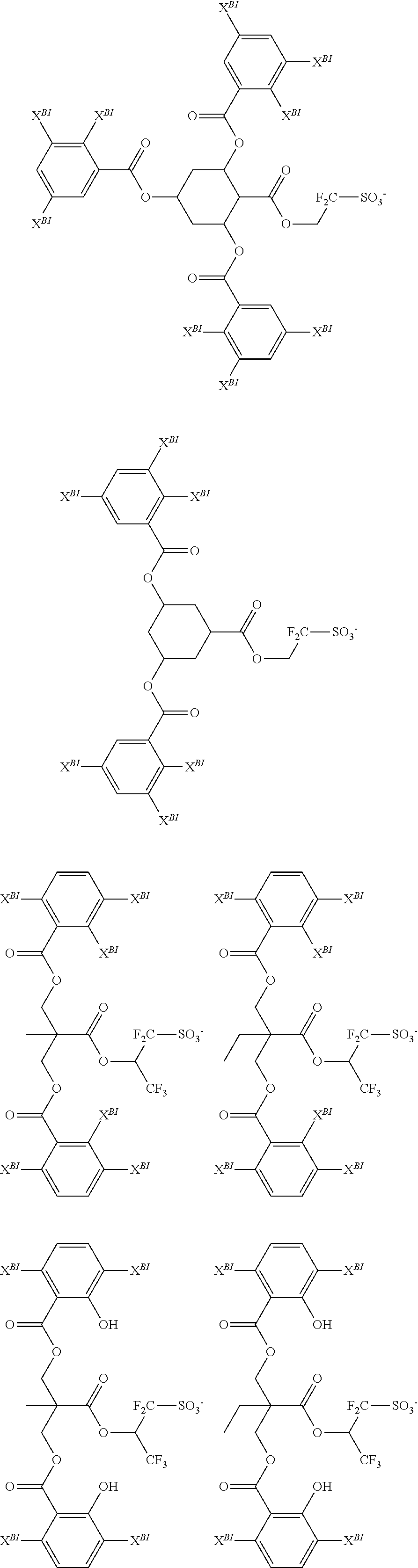

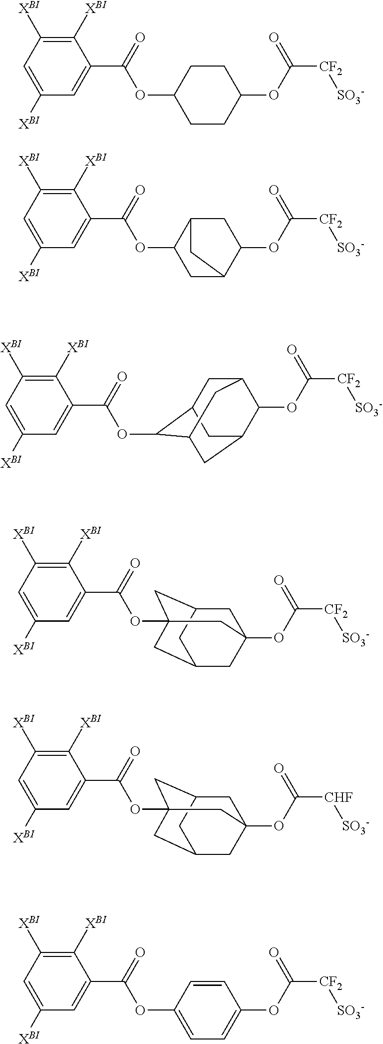

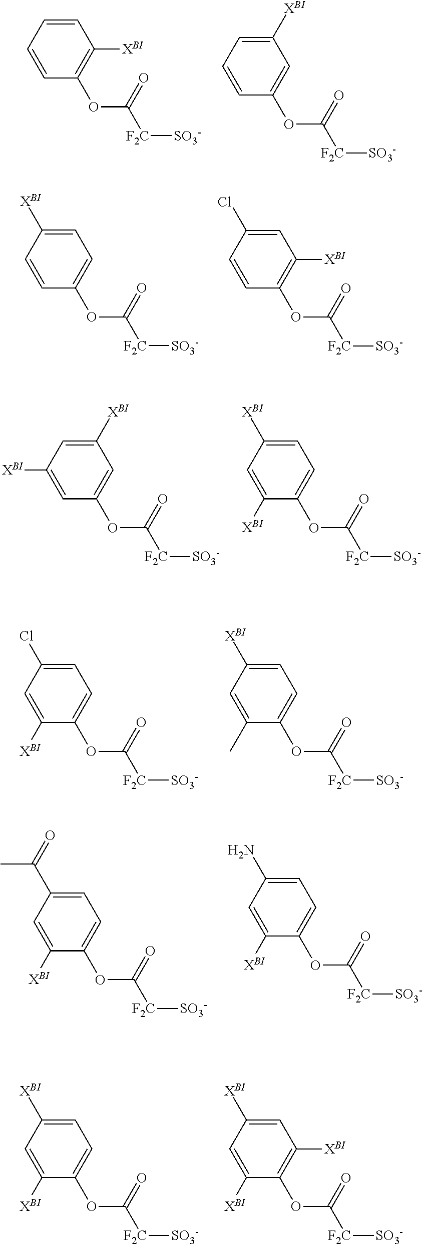

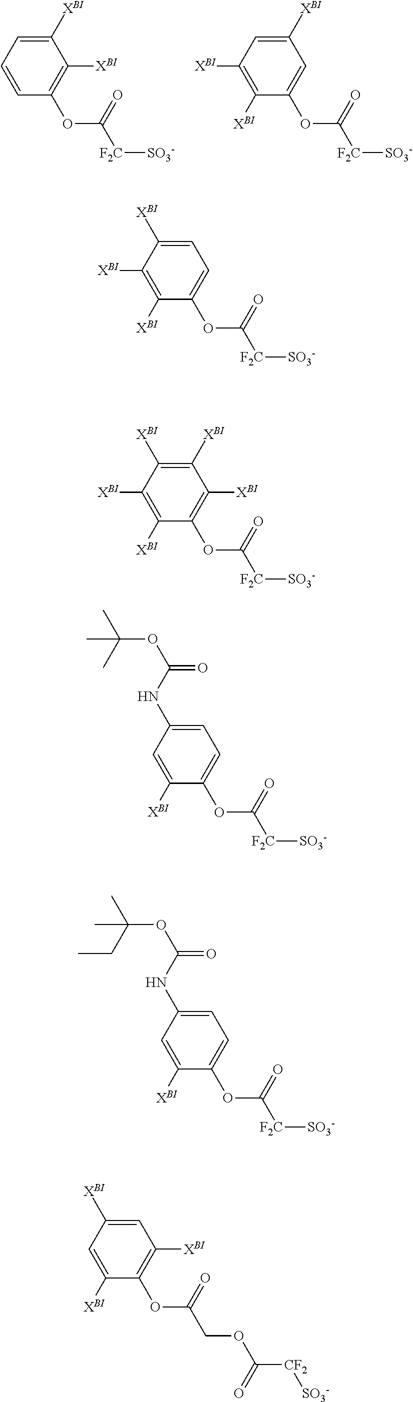

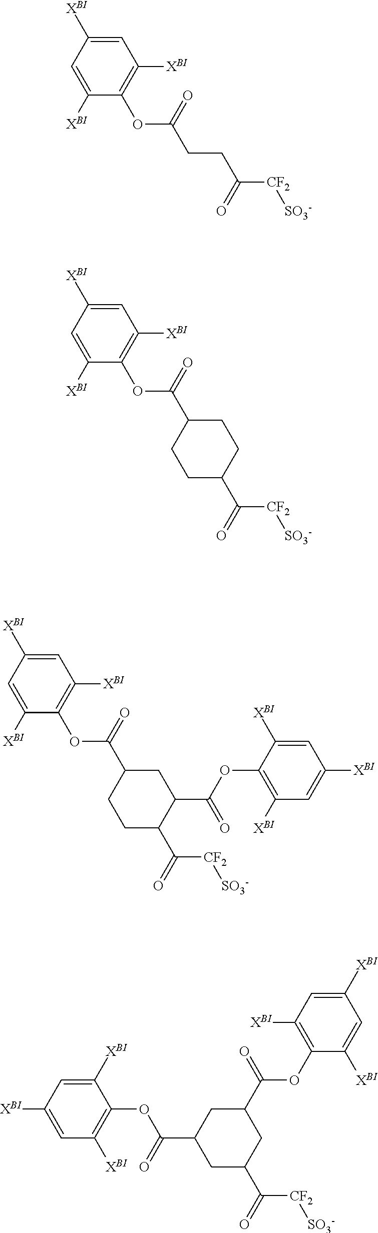

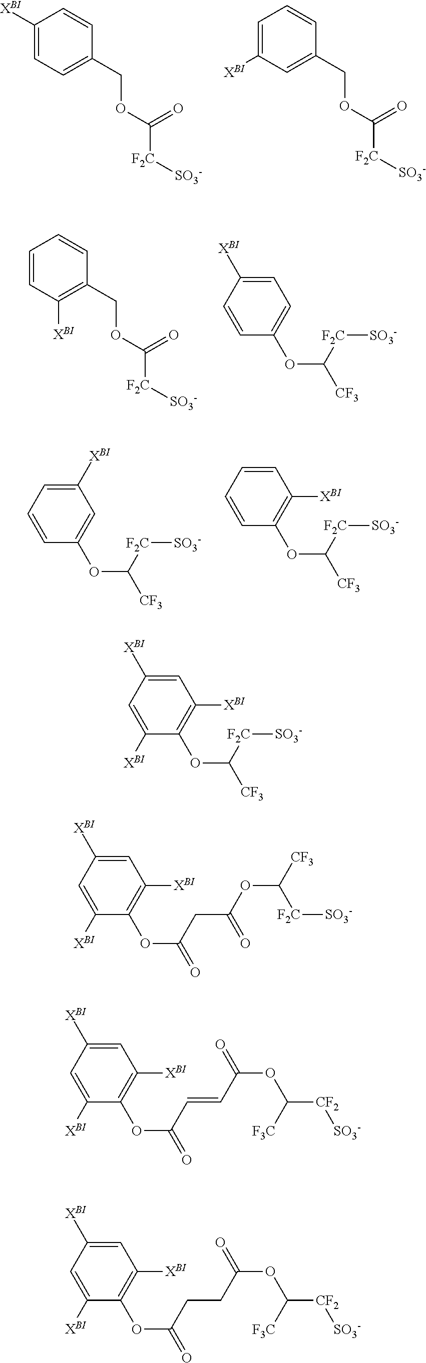

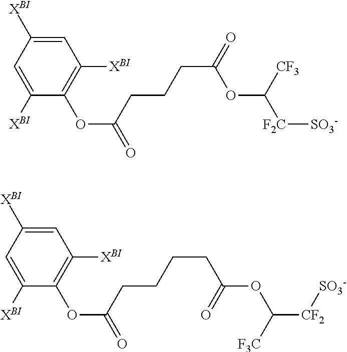

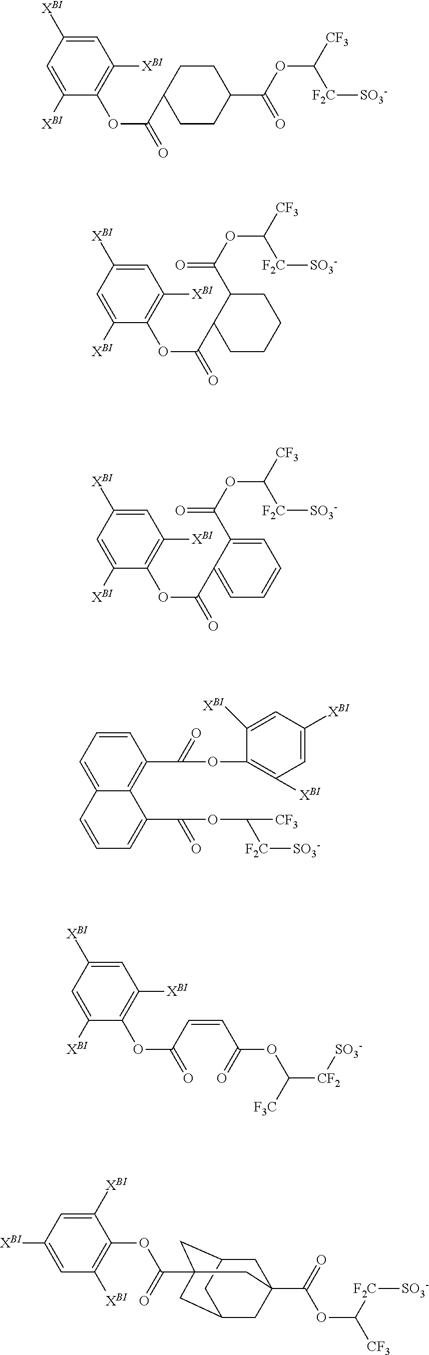

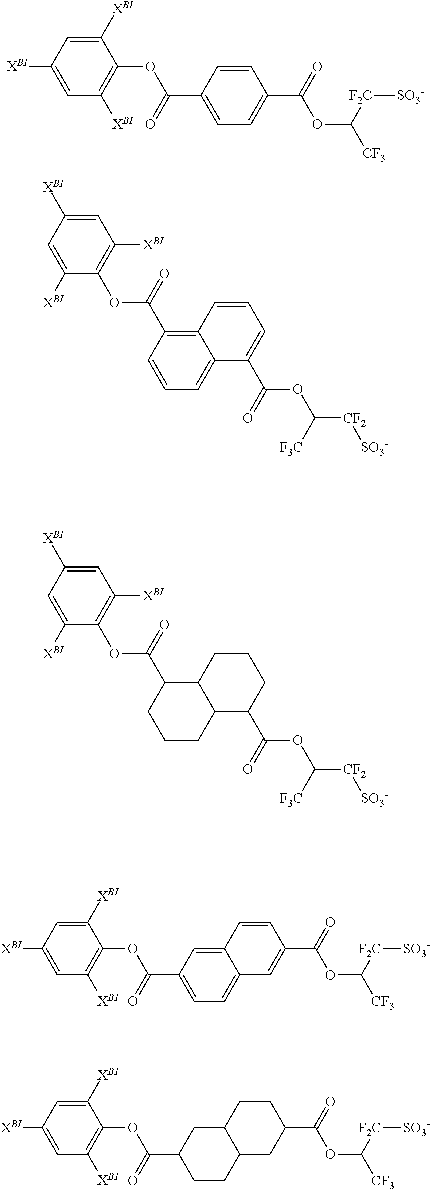

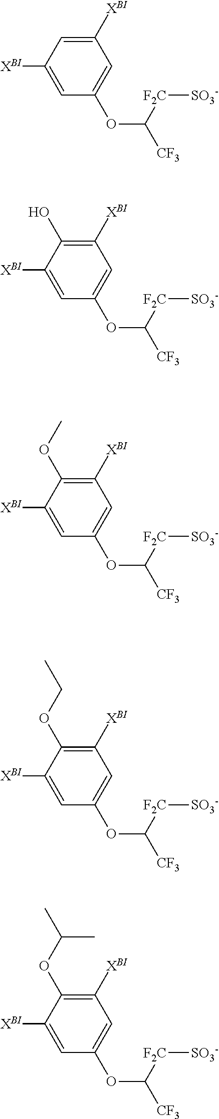

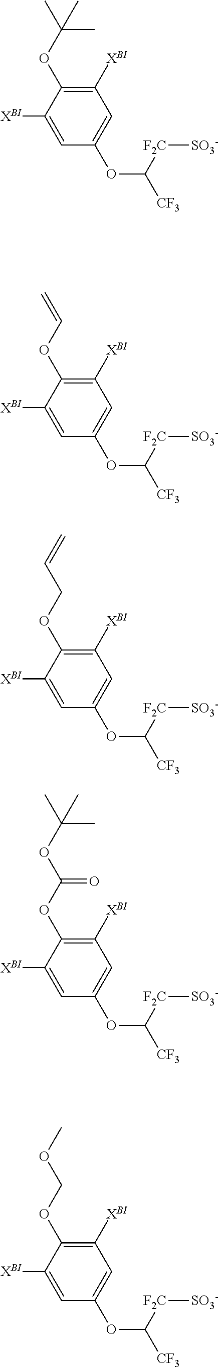

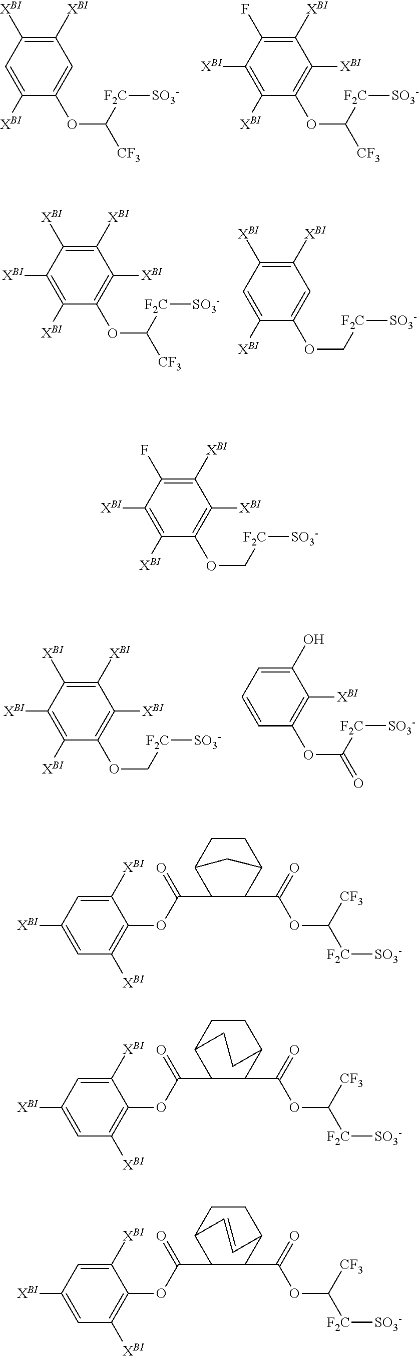

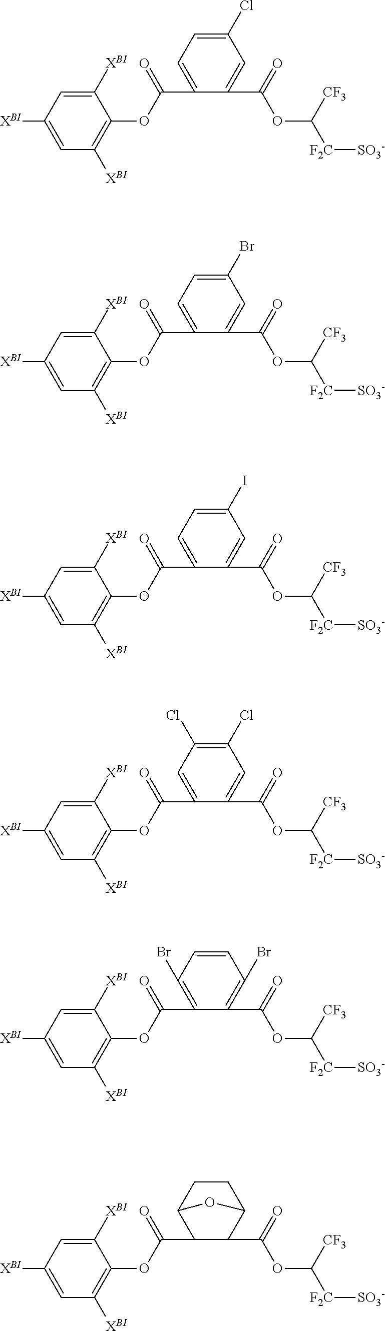

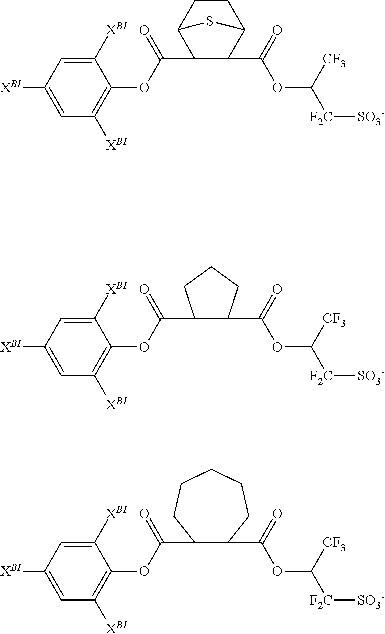

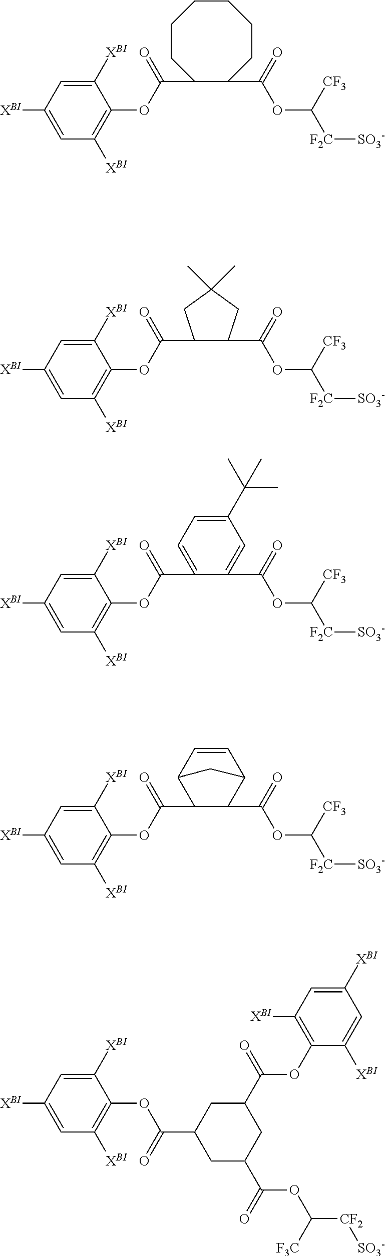

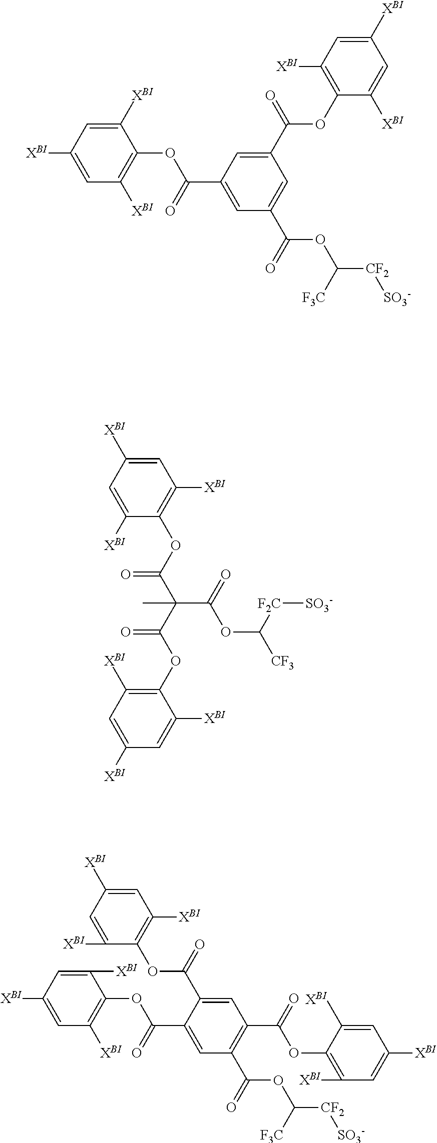

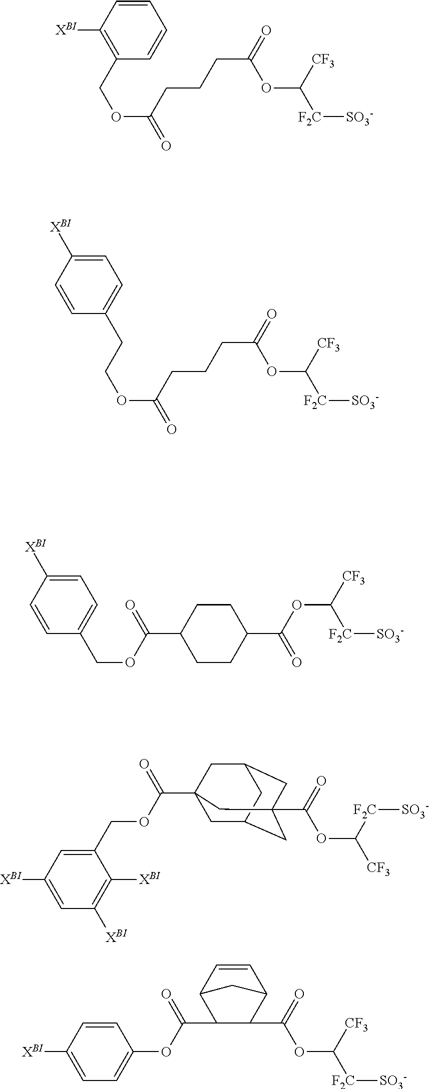

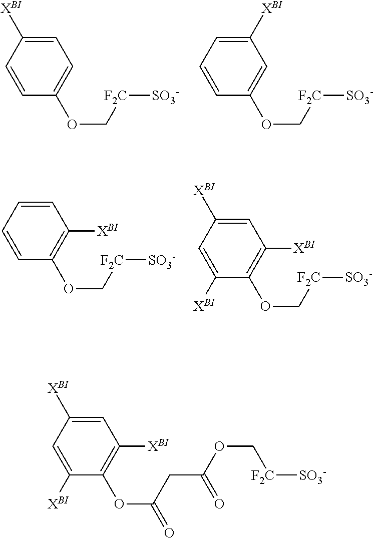

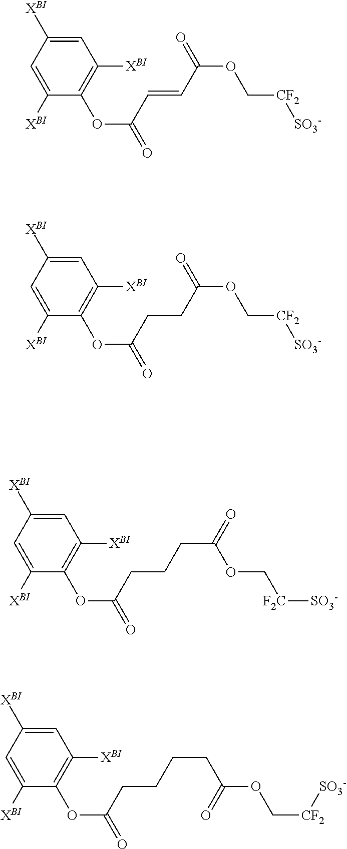

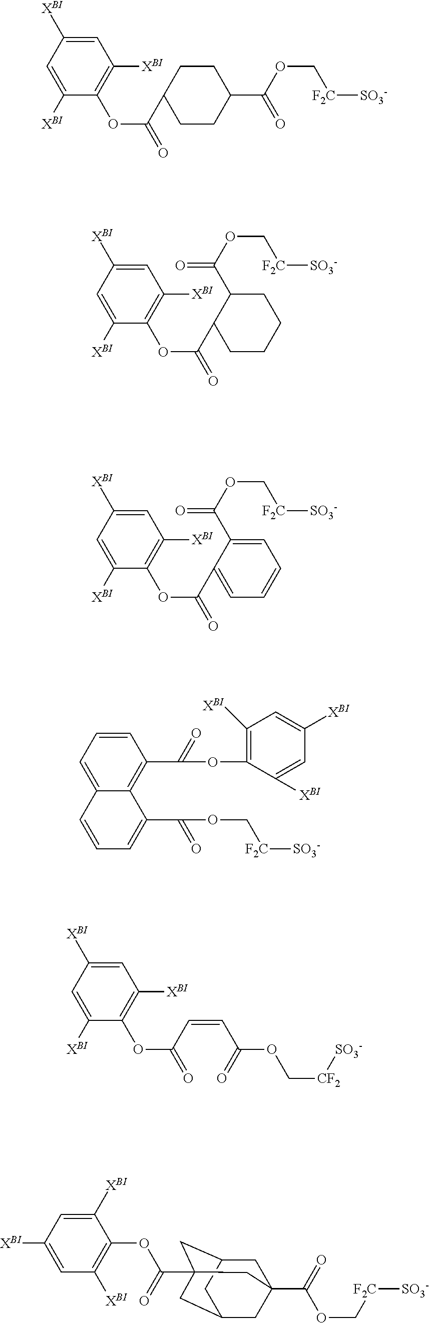

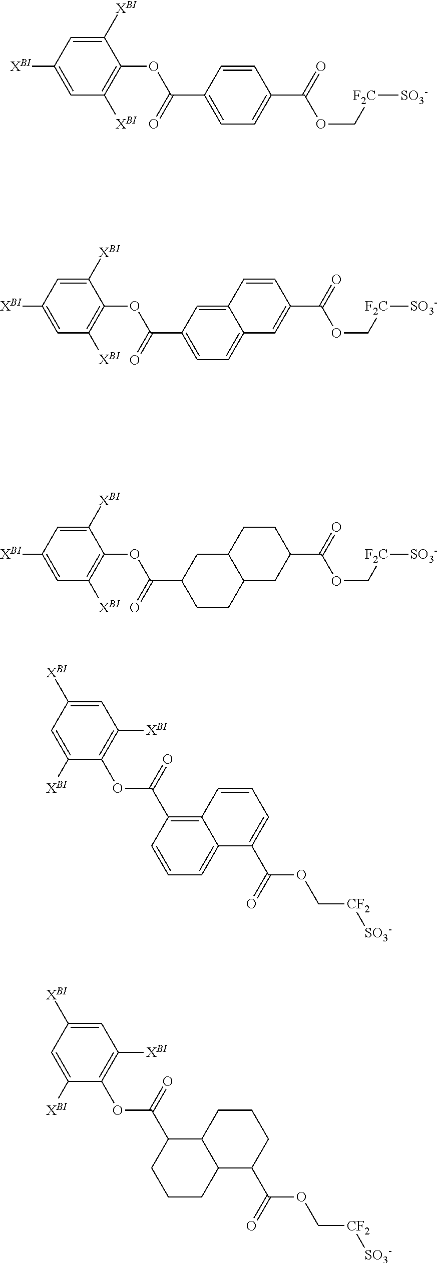

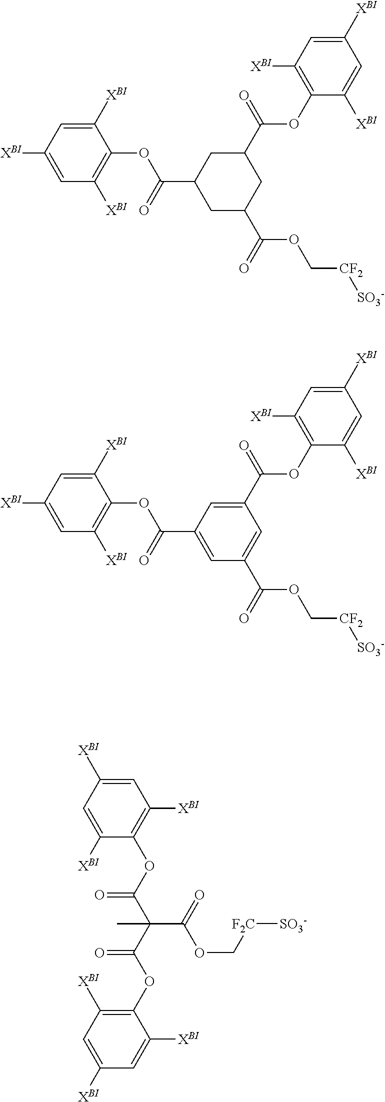

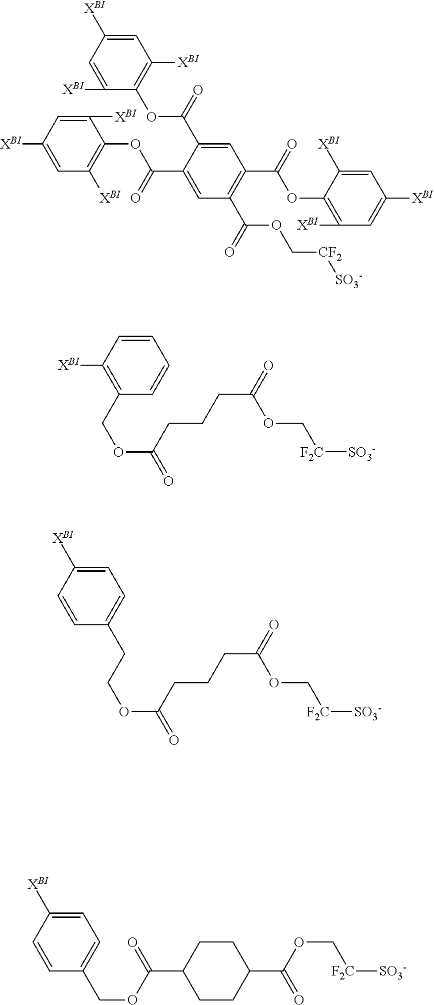

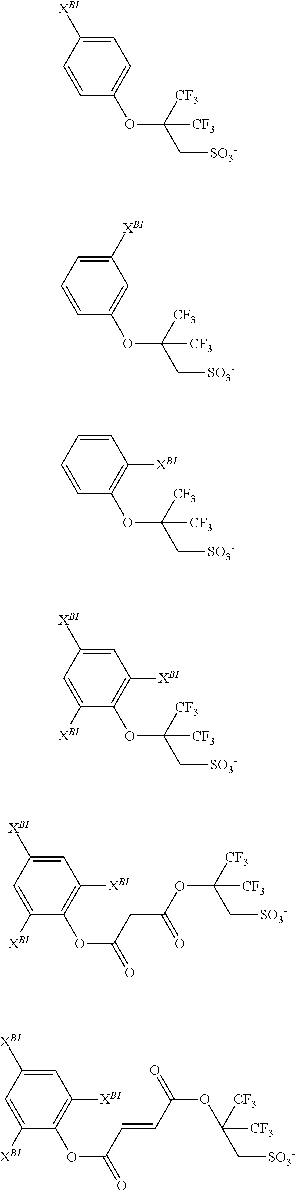

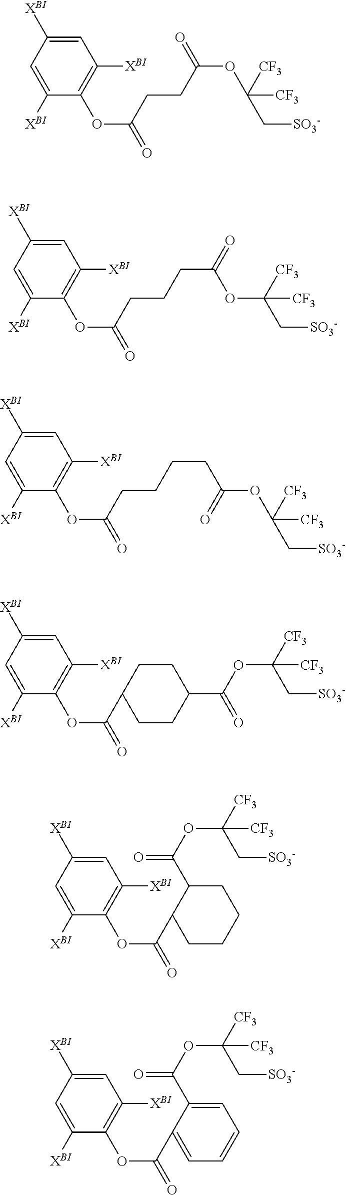

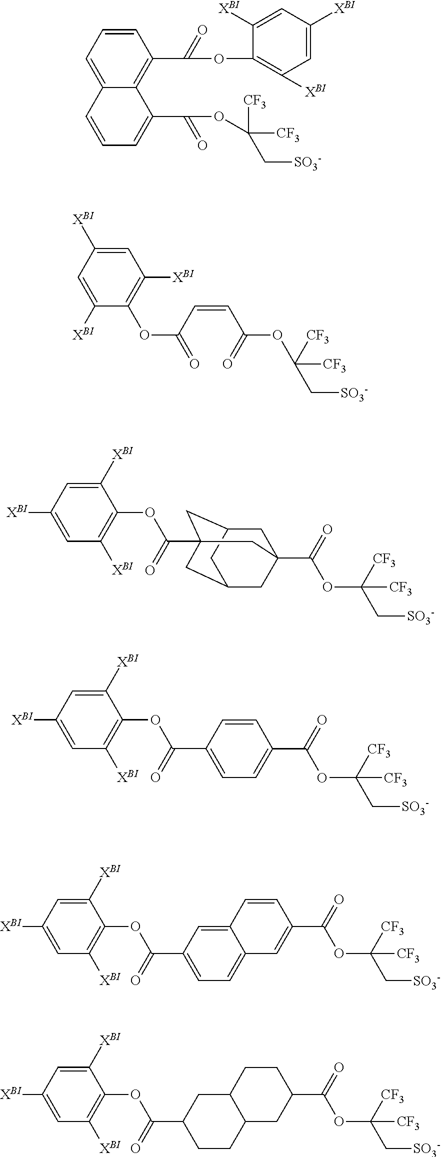

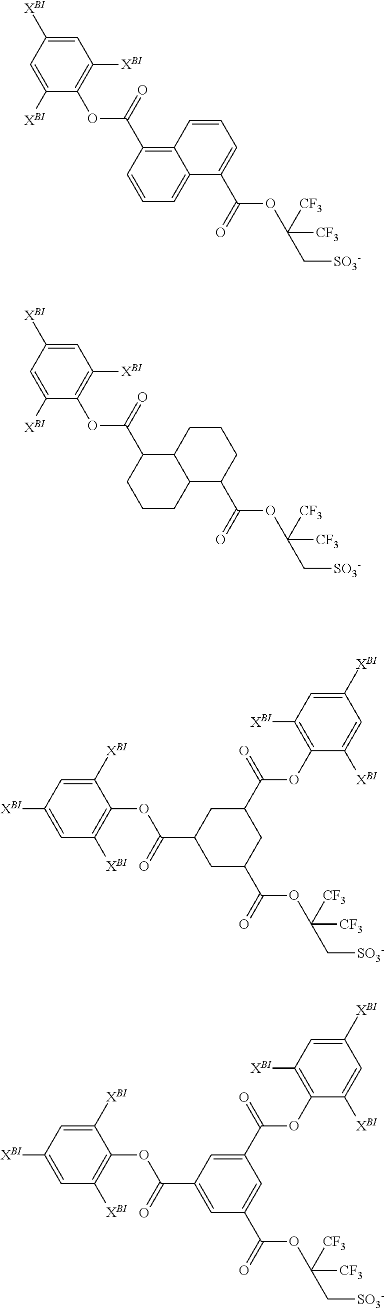

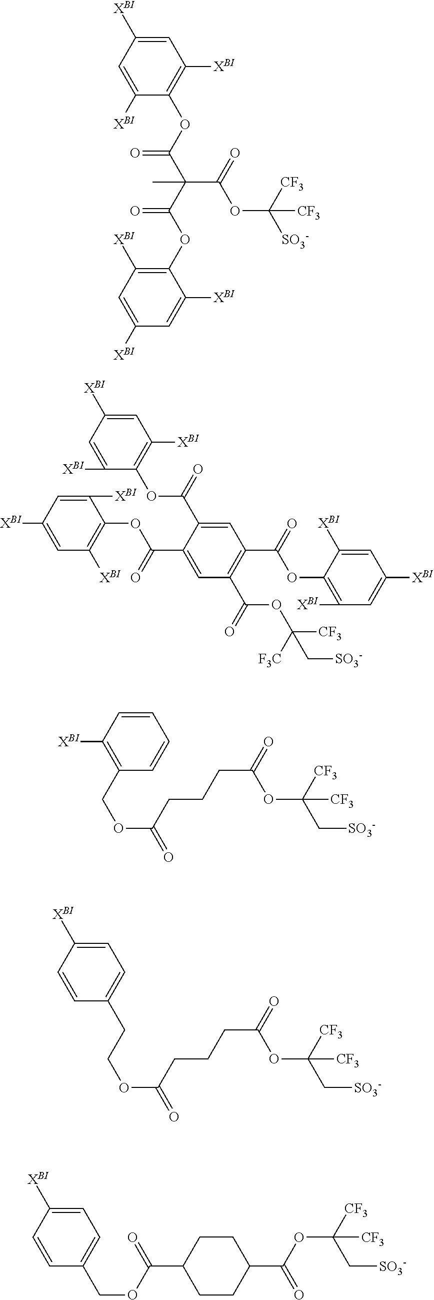

Other useful examples of the non-nucleophilic counter ion include fluorobenzenesulfonic acid anions having an iodized aromatic ring bonded thereto as described in JP 6648726, anions having an acid-catalyzed decomposition mechanism as described in WO 2021/200056 and JP-A 2021-70692, anions having a cyclic ether group as described in JP-A 2018-180525 and JP-A 2021-35935, and anions as described in JP-A 2018-92159.

Further useful examples of the non-nucleophilic counter ion include bulky fluorine free benzenesulfonic acid anions as described in JP-A 2006-276759, JP-A 2015-117200, JP-A 2016-65016, and JP-A 2019-202974; and fluorine-free benzenesulfonic acid or alkylsulfonic acid anions having an iodized aromatic group bonded thereto as described in JP 6645464.

As the non-nucleophilic counter ion, bissulfonic acid anions as described in JP-A 2015-206932, sulfonamide or sulfonimide anions having sulfonic acid side and different side as described in WO 2020/158366, and anions having a sulfonic acid side and a carboxylic acid side as described in JP-A 2015-24989 can be further used.

In formulae (c2) and (c3), d1 and d2 are each independently 0, 1, 2, or 3, and preferably 1.

In formula (c4), e1 is 0 or 1, e2 is 0, 1, 2, 3, or 4, e3 is 0, 1, 2, 3, or 4. Provided that 0≤e2+e3≤4 when e1 is 0 and 0≤e2+e3≤6 when e1 is 1.

In formulae (c2), (c3), and (c4), L1 is a single bond, an ether bond, an ester bond, a carbonyl group, a sulfonate ester bond, a sulfonamide bond, a carbonate bond, or a carbamate bond. Among them, from the viewpoint of synthesis, an ether bond, an ester bond, and a carbonyl group are preferred, and an ester bond and a carbonyl group are more preferred.

In formula (c2), Rf¹ and Rf² are each independently a fluorine atom or a C₁-C₆ fluorinated saturated hydrocarbyl group. Among them, Rf¹ and Rf² are each preferably a fluorine atom in order to increase the acid strength of the generated acid. Rf³ and Rf⁴ are each independently a hydrogen atom, a fluorine atom, or a C₁-C₆ fluorinated saturated hydrocarbyl group. Among them, at least one of Rf³ and Rf⁴ is preferably a trifluoromethyl group for improving solvent solubility.

In formula (c3), Rf⁵ and Rf⁶ are each independently a hydrogen atom, a fluorine atom, or a C₁-C₆ fluorinated saturated hydrocarbyl group. It is excluded that all Rf⁵ and Rf⁶ are hydrogen atoms at the same time. Among them, at least one of Rf⁵ and Rf⁶ is preferably a trifluoromethyl group for improving solvent solubility.

In formula (c4), Rf⁷ is a fluorine atom, a C₁-C₆ fluorinated alkyl group, a C₁-C₆ fluorinated alkoxy group, or a C₁-C₆ fluorinated alkylthio group. Rf⁷ is preferably a fluorine atom, a trifluoromethyl group, a difluoromethyl group, a trifluoromethoxy group, a difluoromethoxy group, a trifluoromethylthio group, or a difluoromethylthio group, and more preferably a fluorine atom, a trifluoromethyl group, or a trifluoromethoxy group. A plurality of Rf⁷ may be identical or different when f is 2, 3, or 4.

In formula (c4), R³³ is a halogen atom other than a fluorine atom or a C₁-C₂₀ hydrocarbyl group which may contain a heteroatom. The hydrocarbyl group may be saturated or unsaturated and straight, branched, or cyclic. Specific examples thereof are as exemplified above for the hydrocarbyl group R¹ in the description of formula (1), but are not limited thereto. A plurality of R³³ may be identical or different when e3 is 2, 3, or 4.

The plurality of R³³ may bond together to form a ring with the carbon atoms to which they are attached when e3 is 2, 3, or 4. Specific examples of the ring thus formed include a cyclopropane ring, a cyclobutane ring, a cyclopentane ring, a cyclohexane ring, a norbornane ring, and an adamantane ring. In the ring, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Specific examples of the anion of the repeat units c2 are shown below, but are not limited thereto. Herein R^{A} is as defined above, and Me is a methyl group.

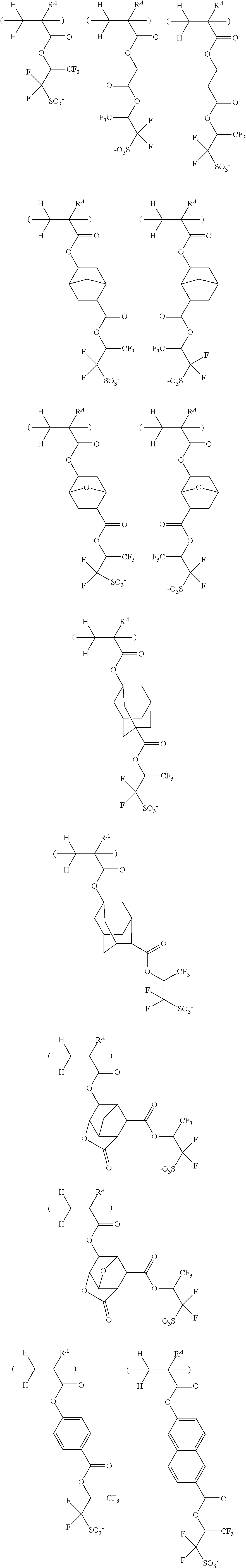

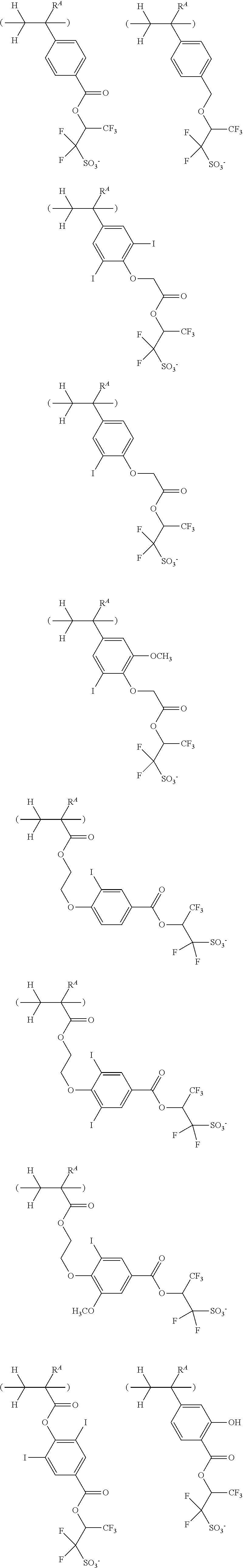

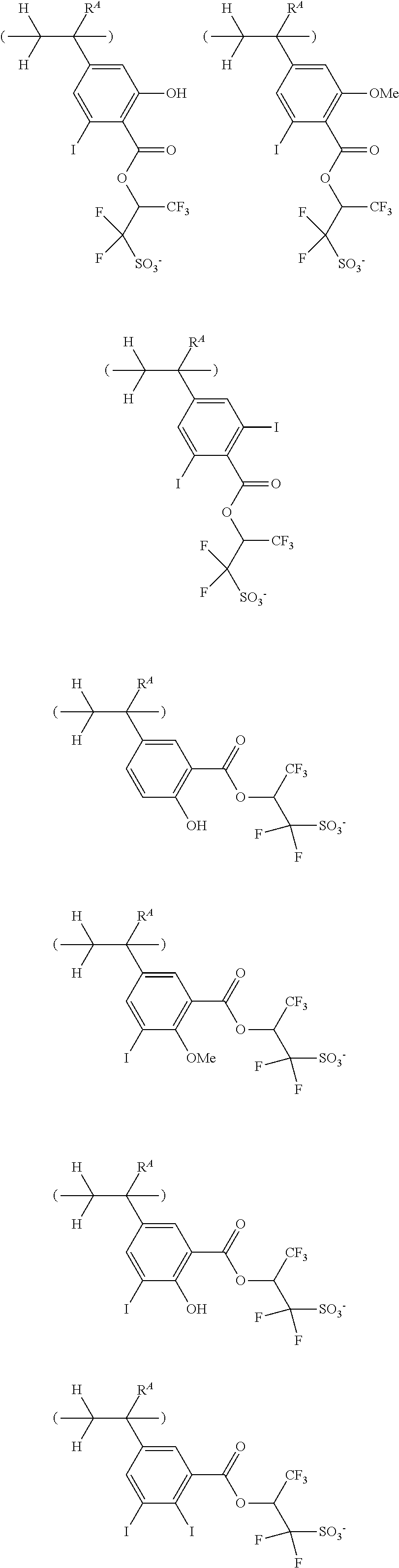

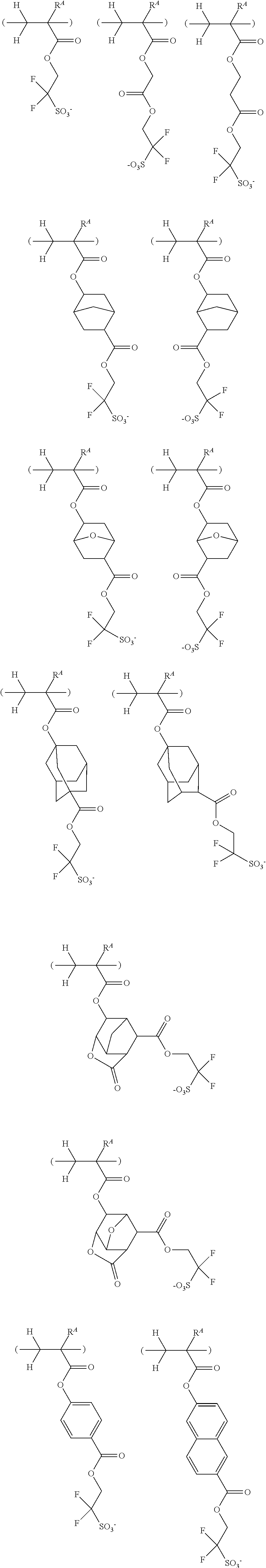

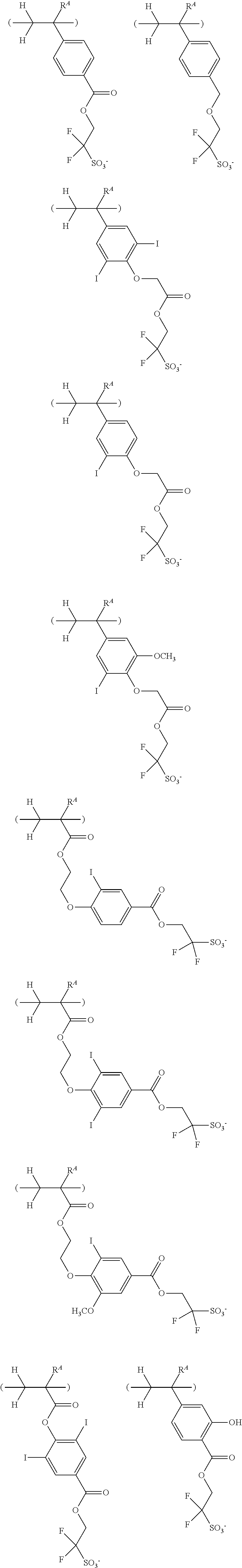

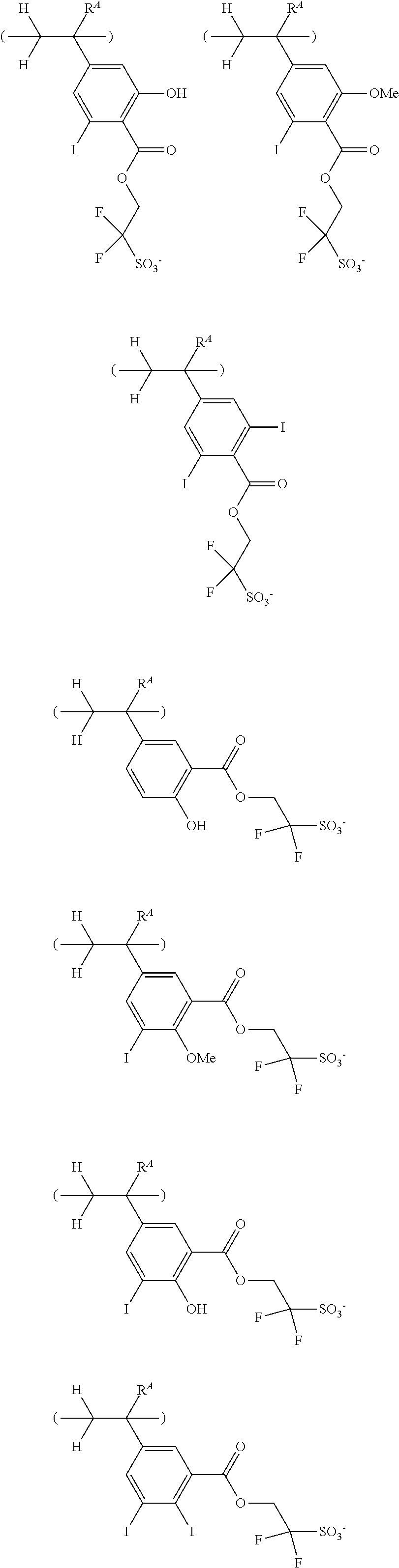

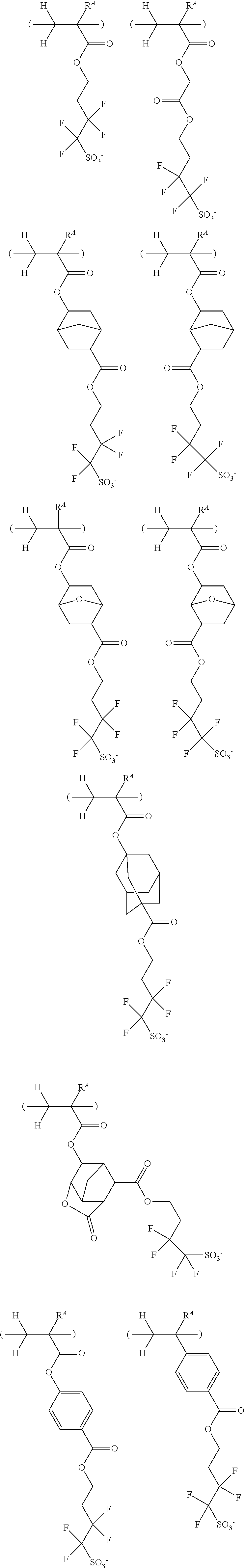

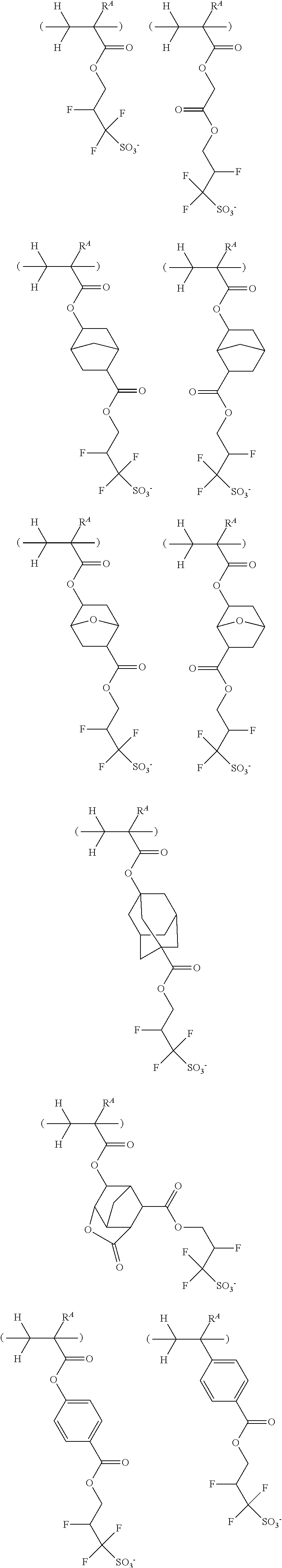

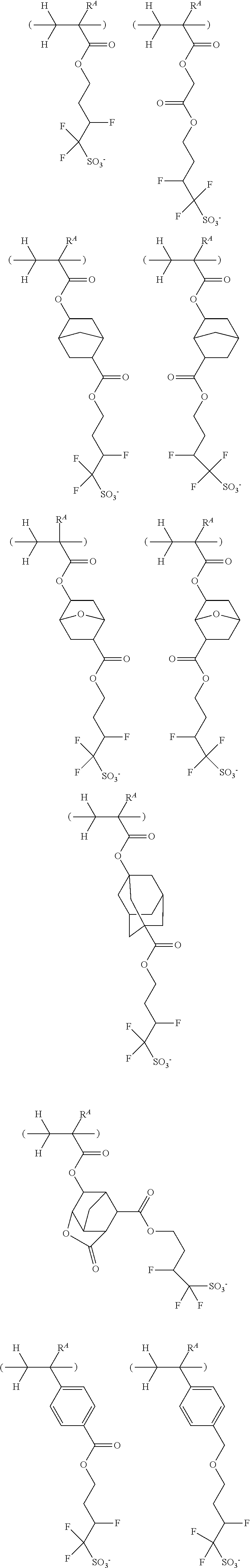

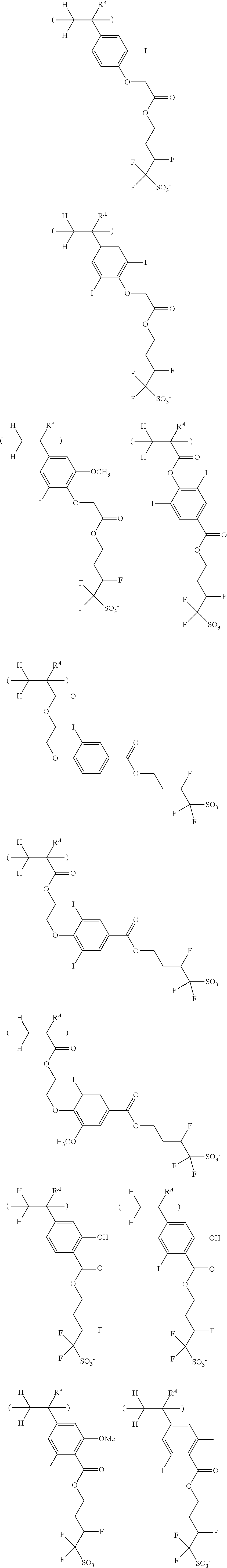

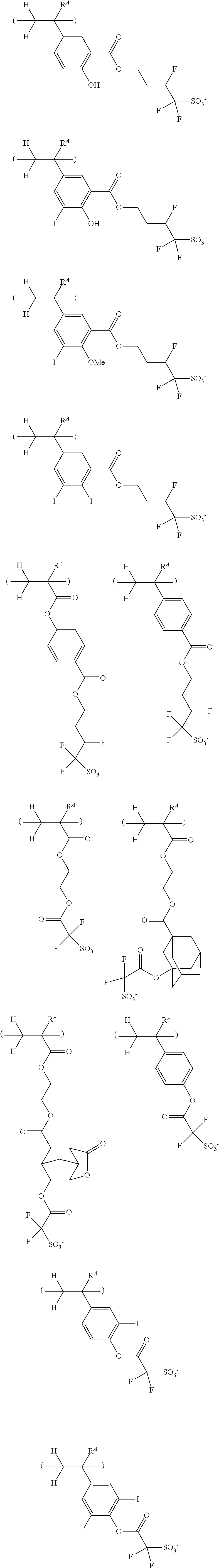

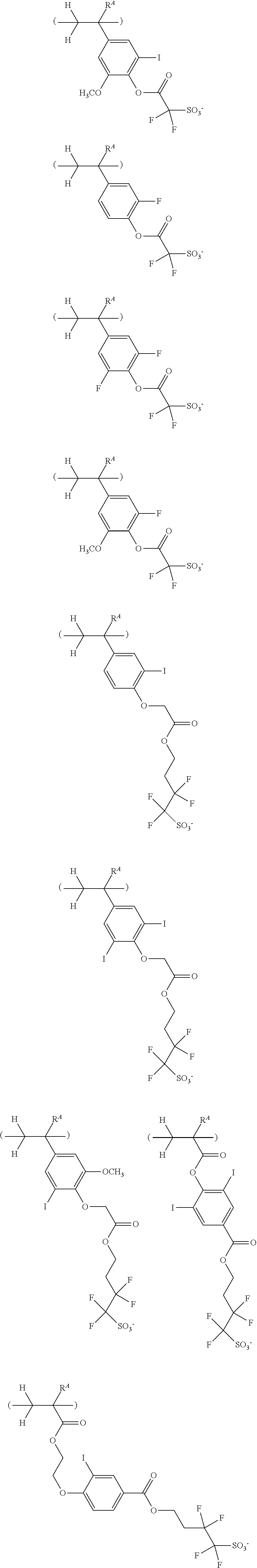

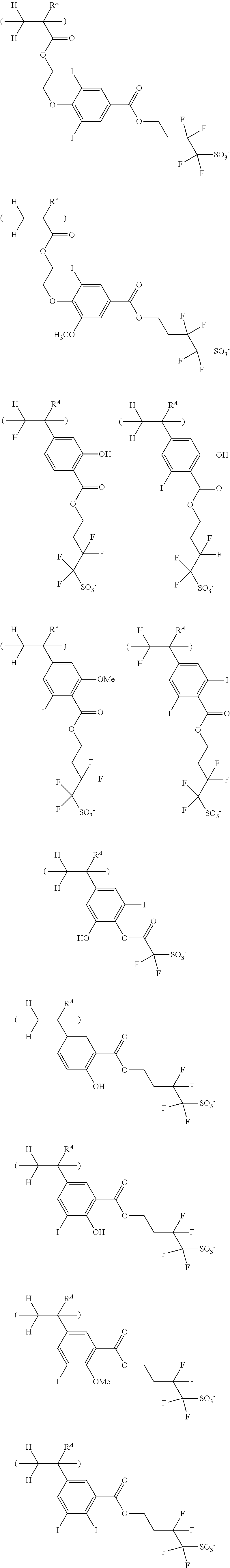

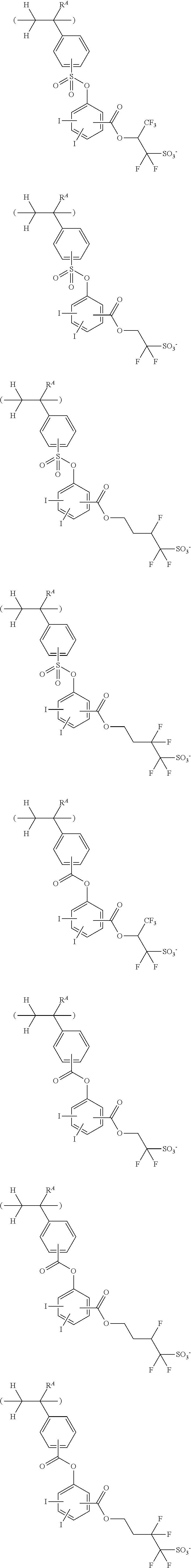

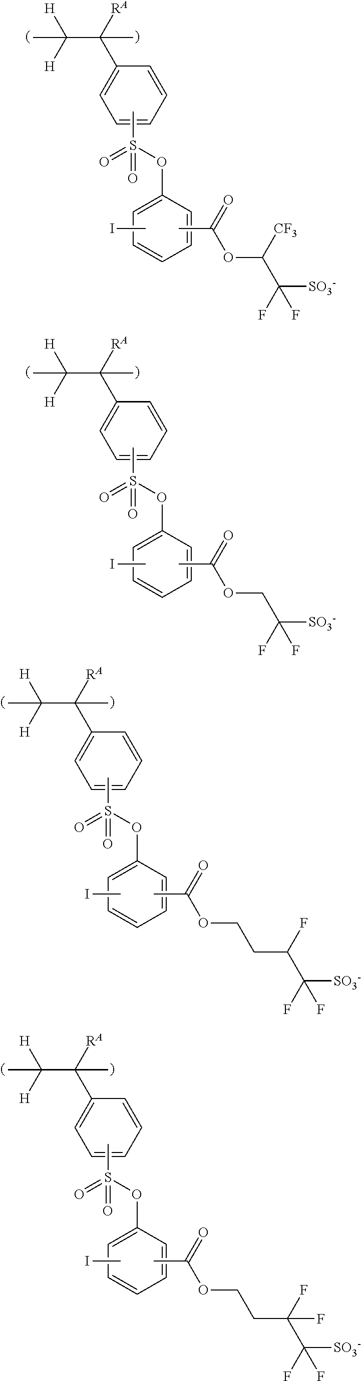

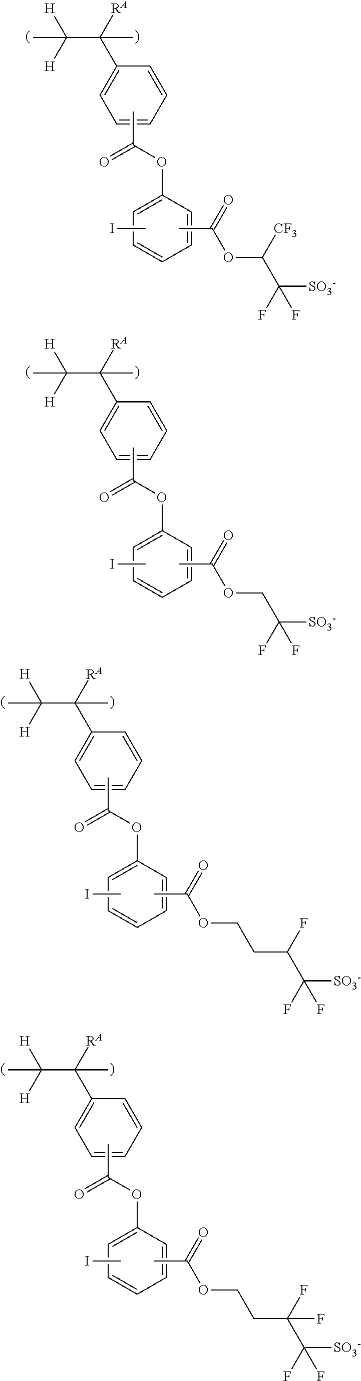

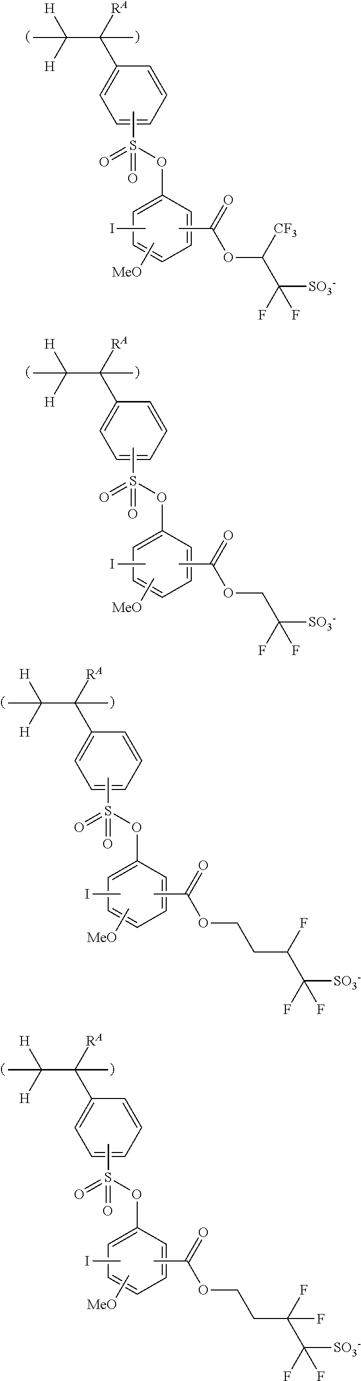

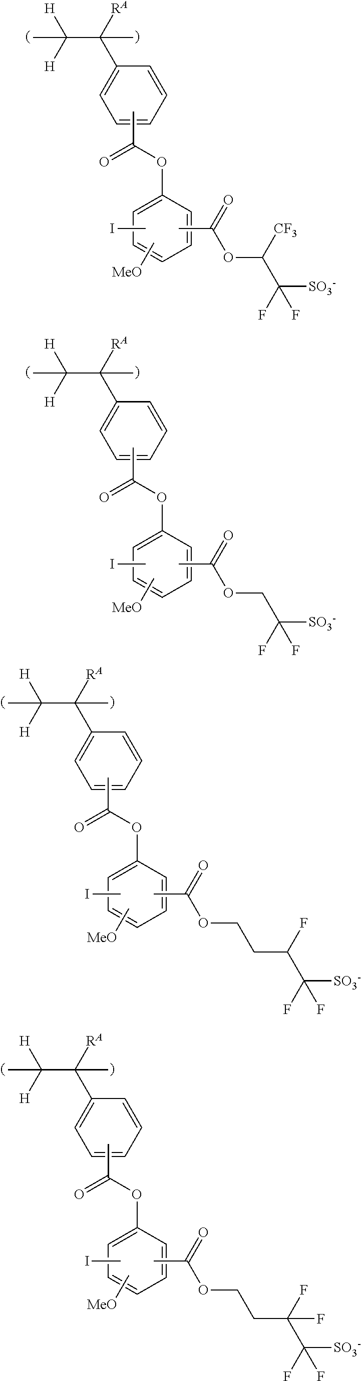

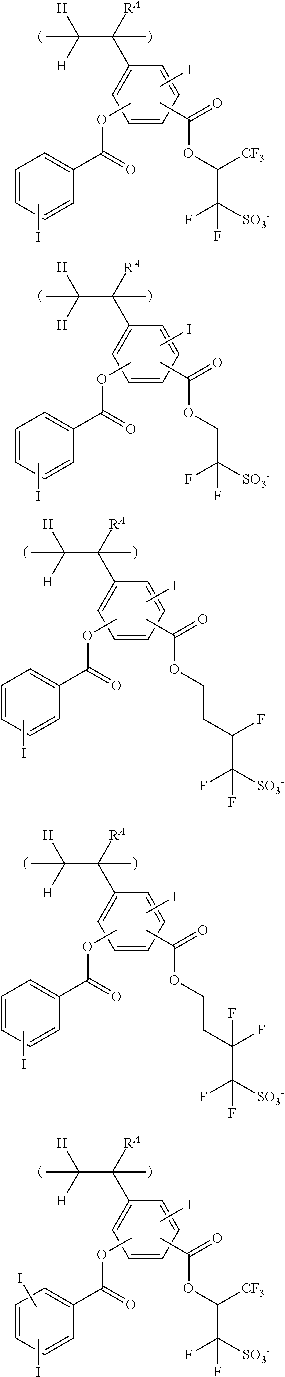

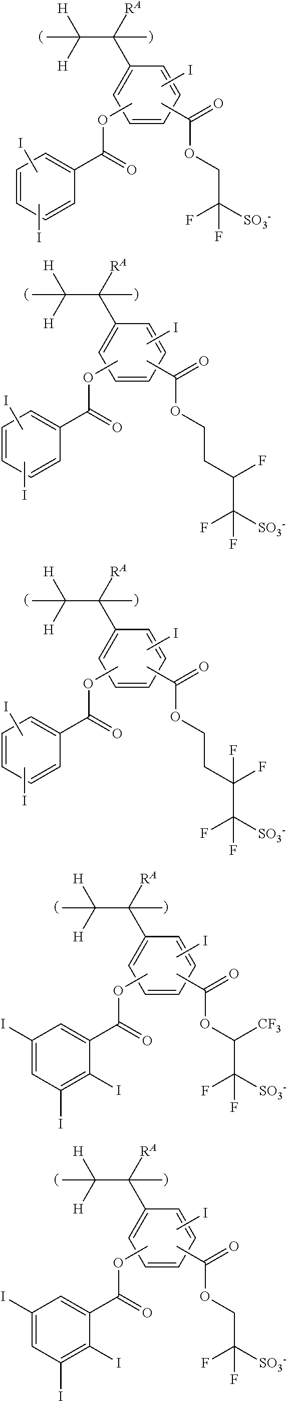

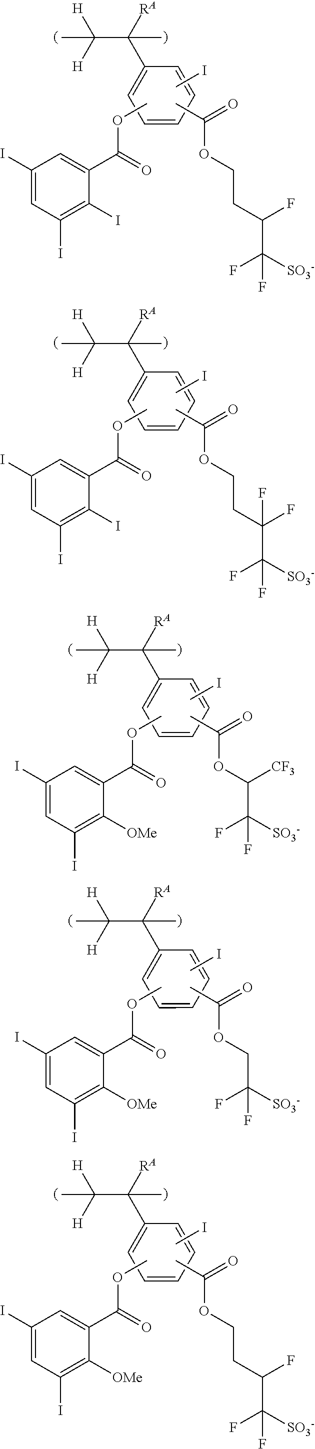

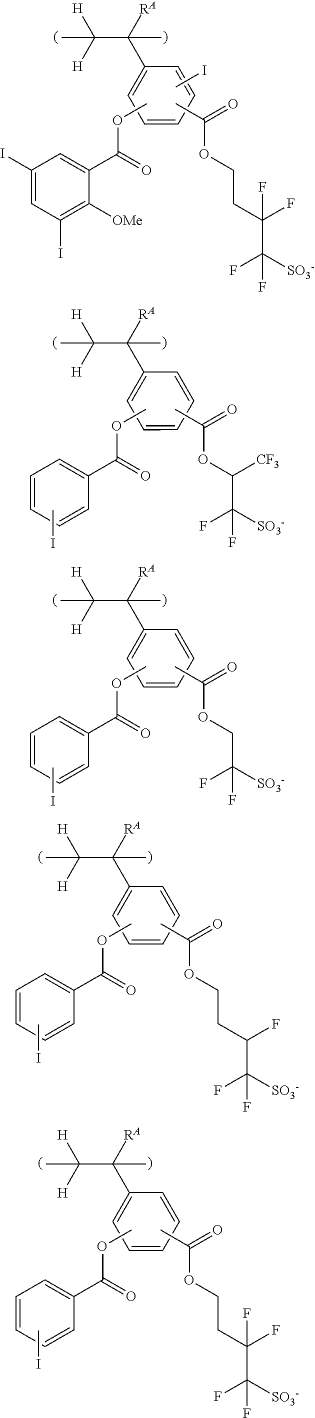

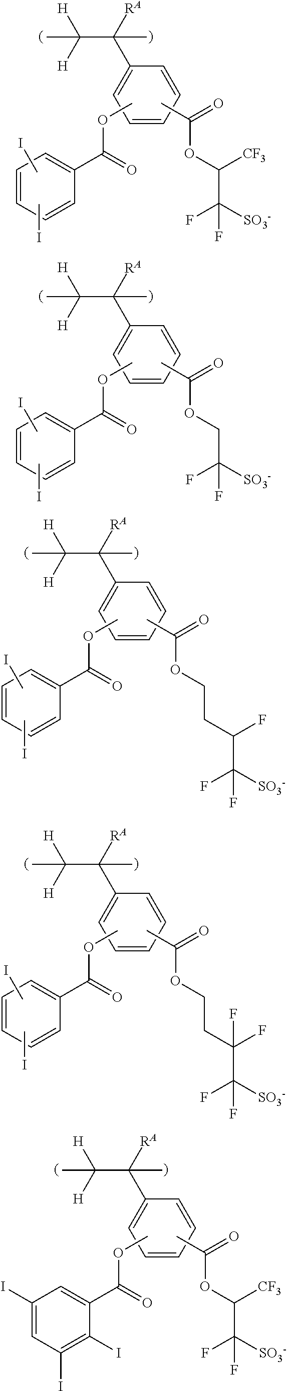

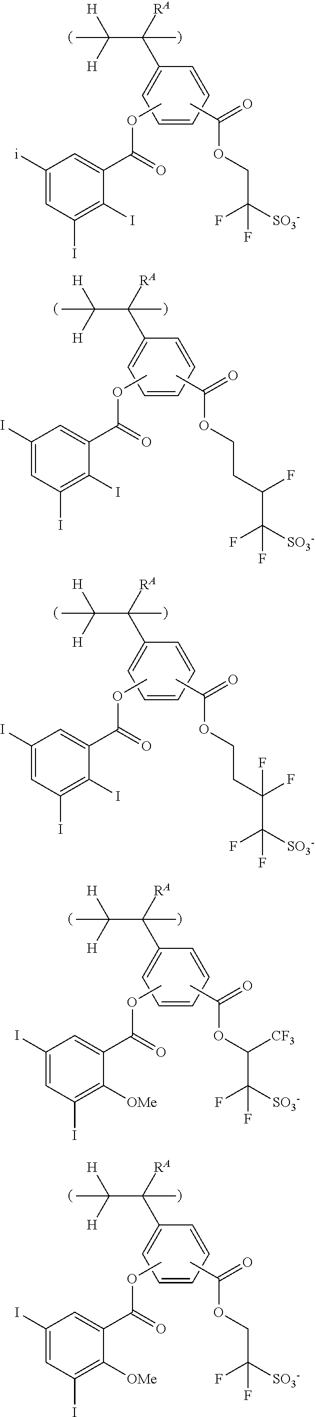

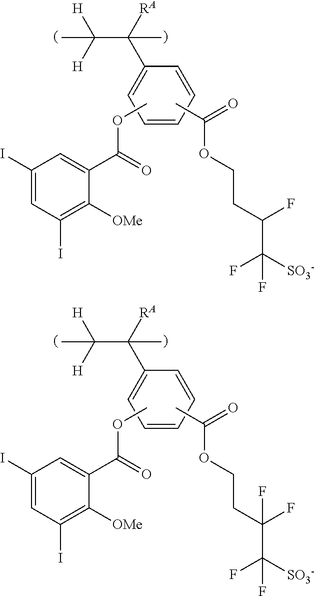

Specific examples of the anion of the repeat units c3 are shown below, but are not limited thereto. Herein R^{A} is as defined above.

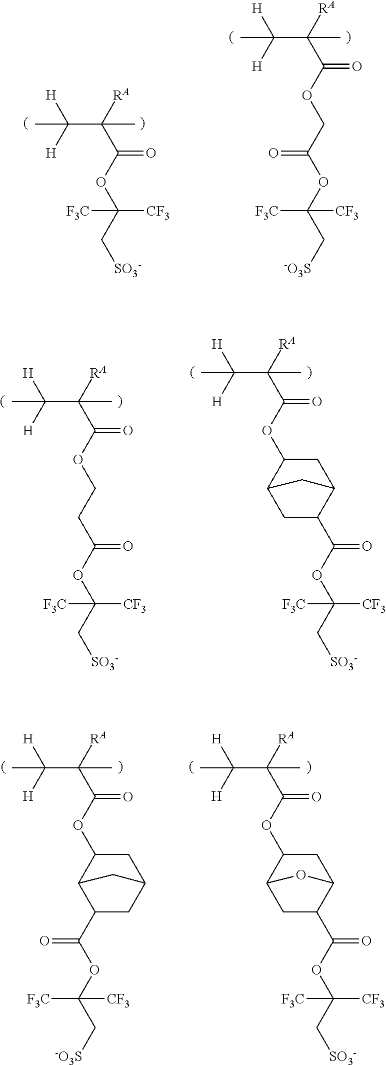

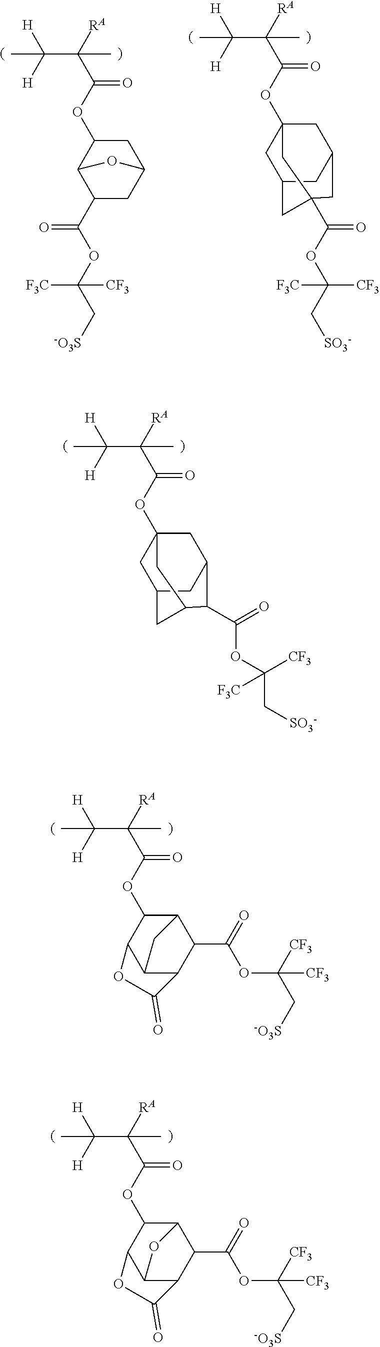

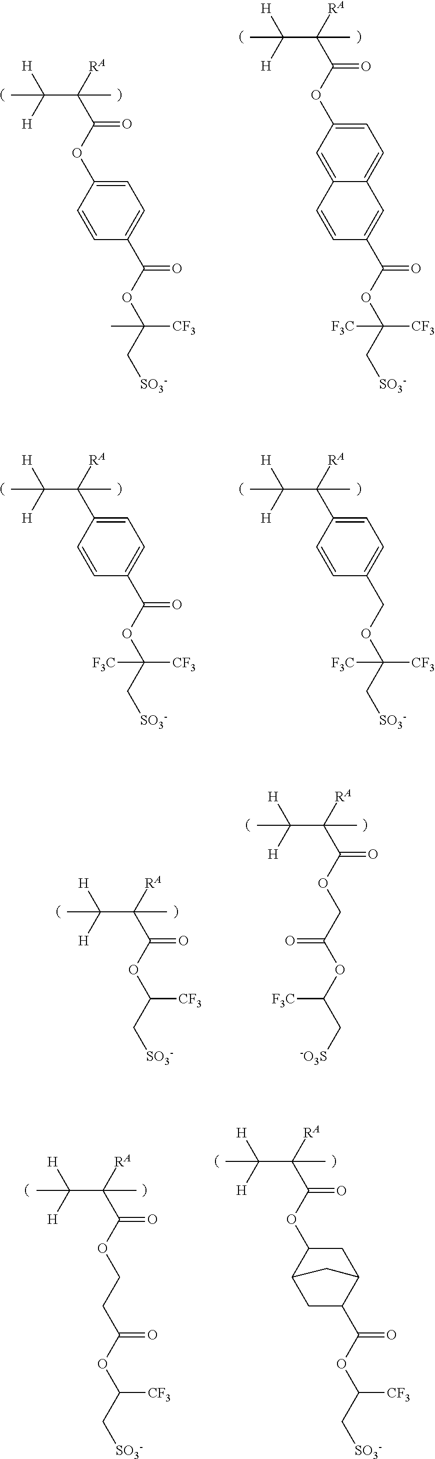

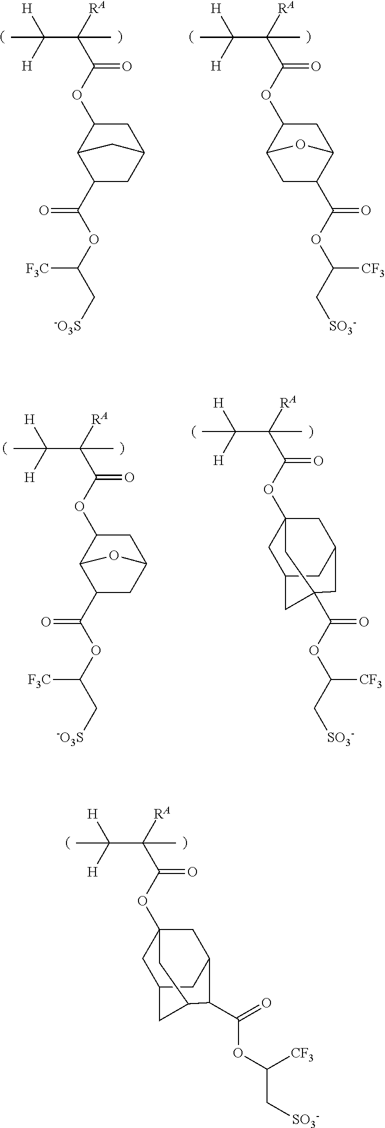

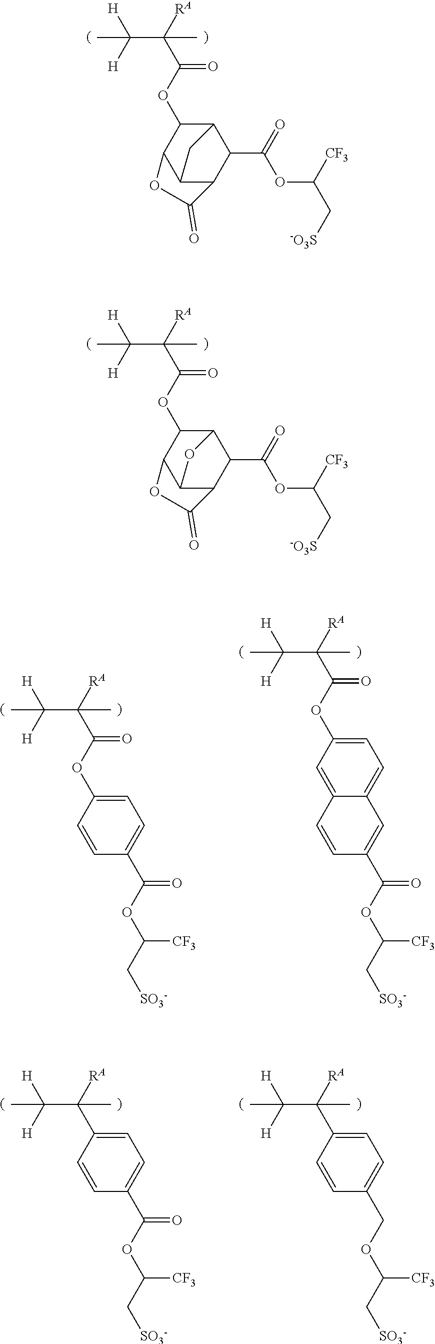

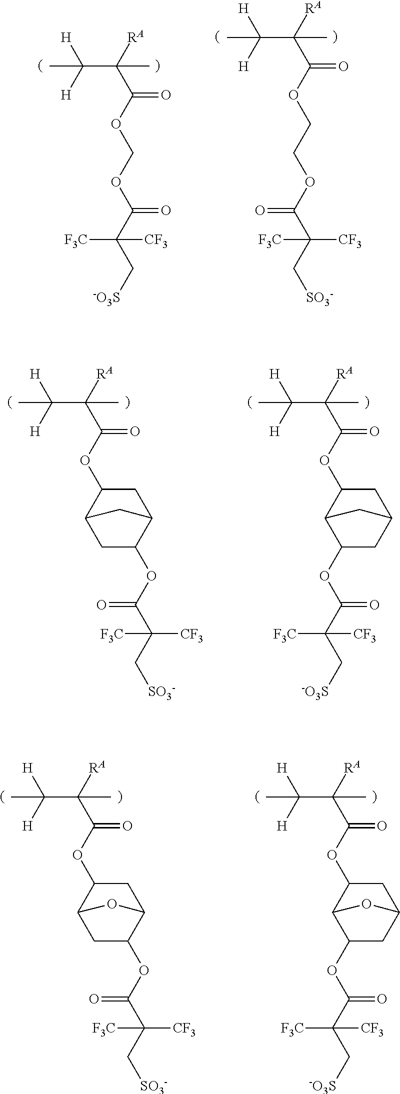

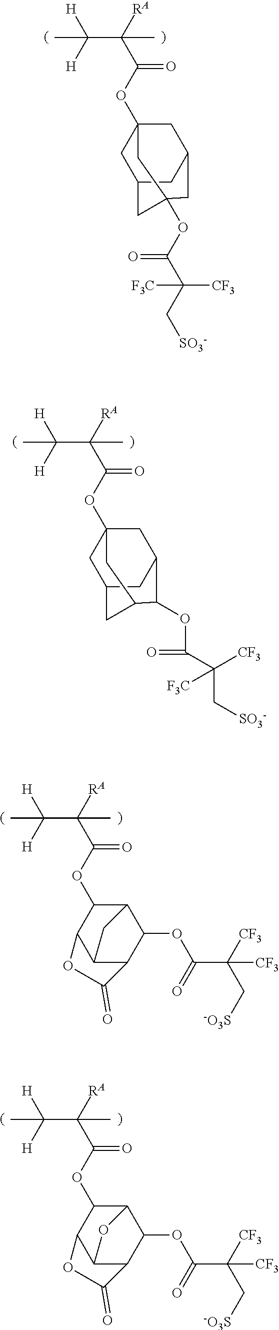

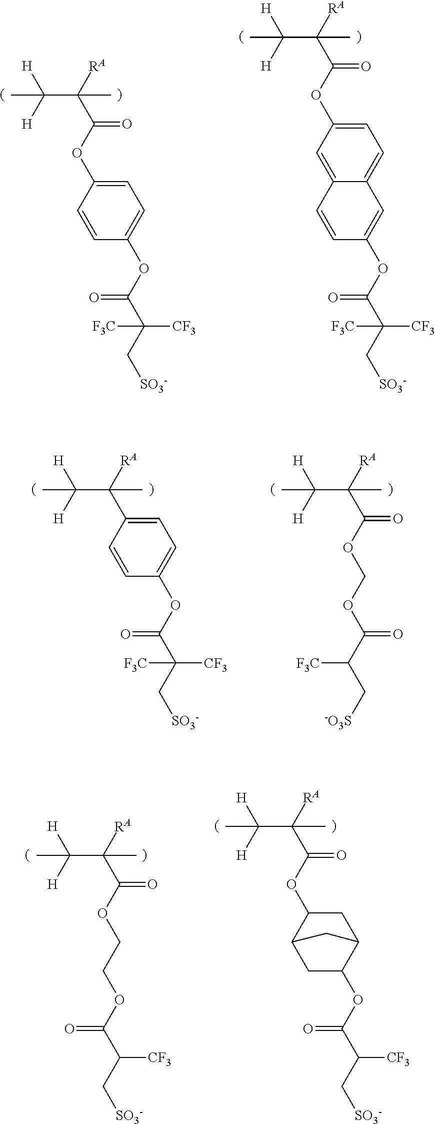

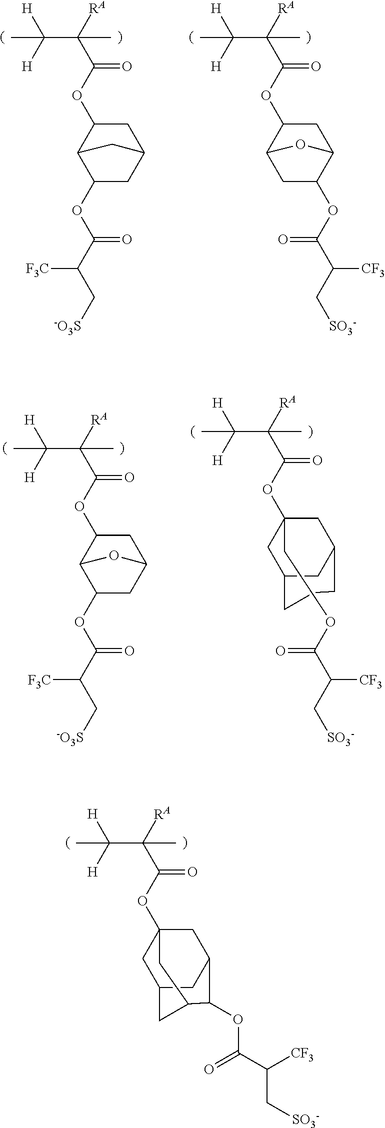

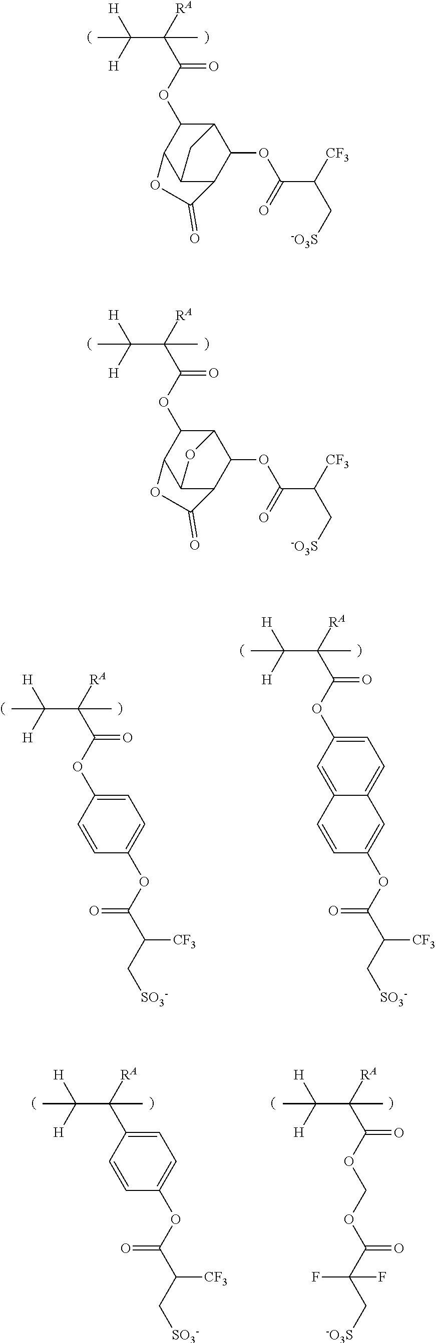

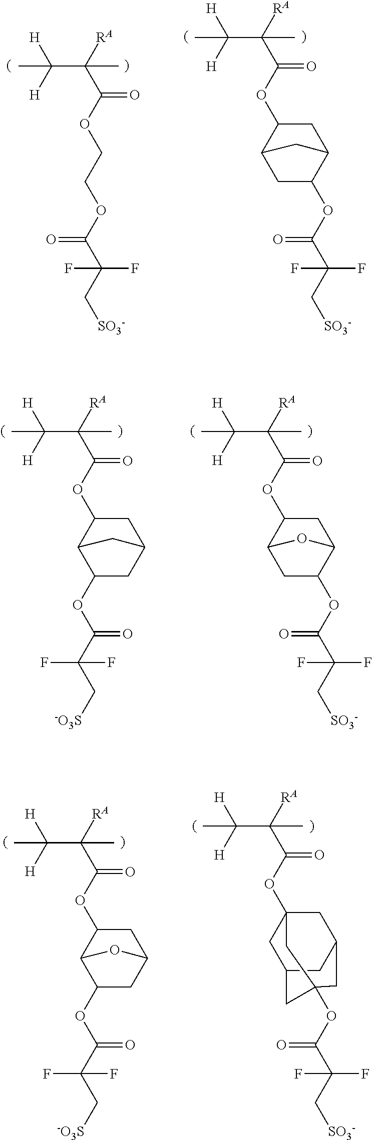

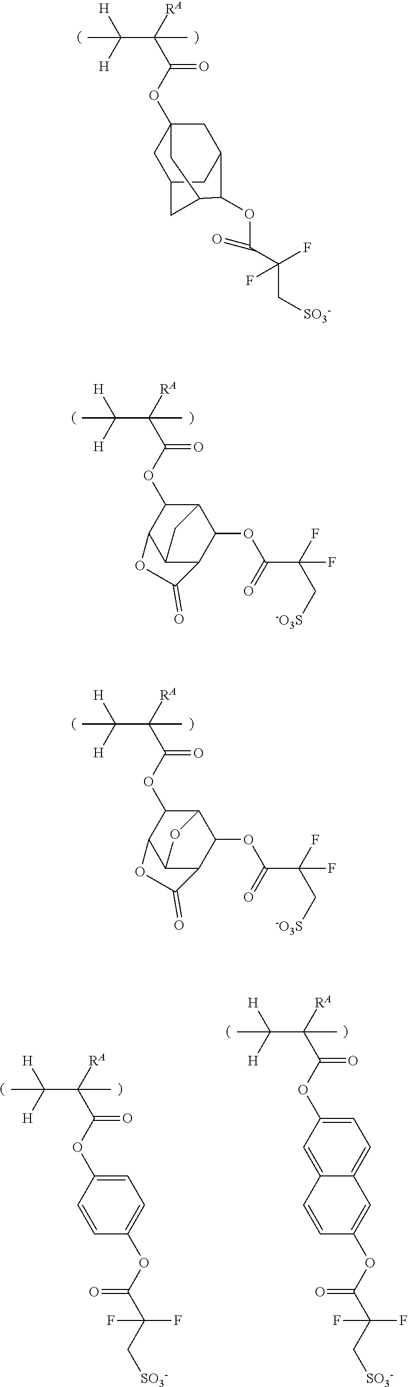

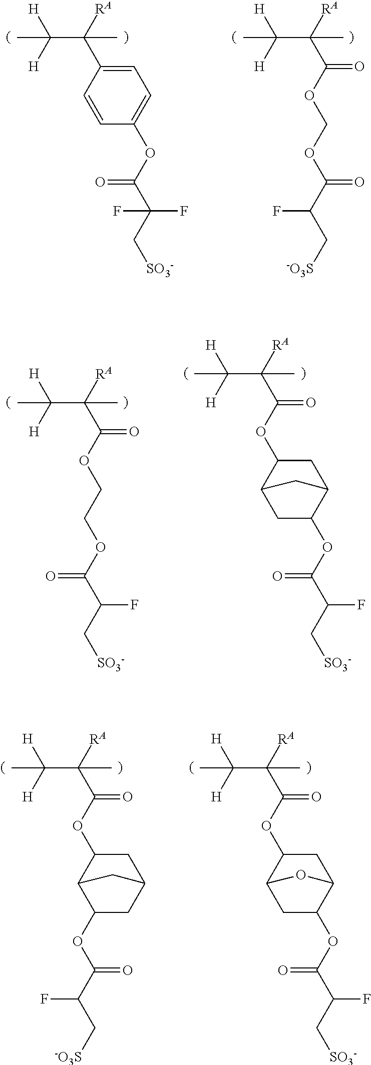

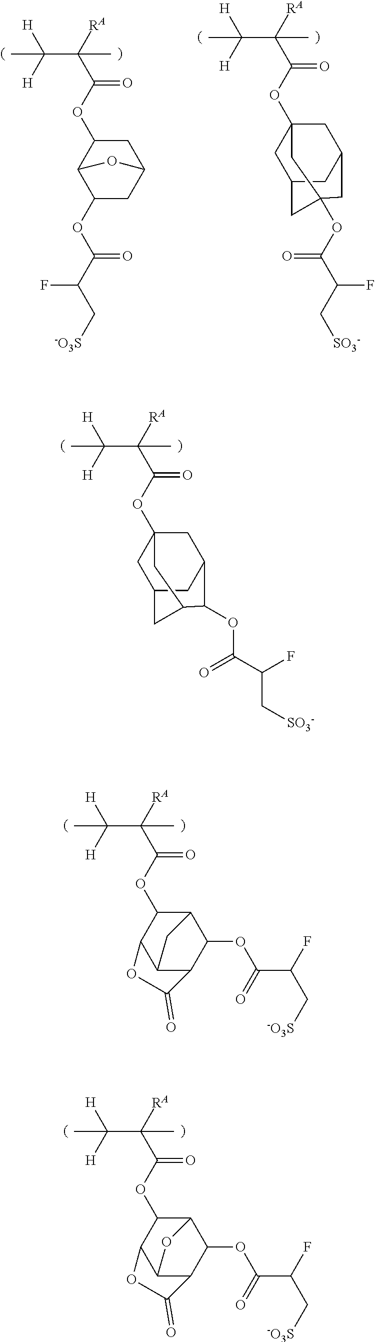

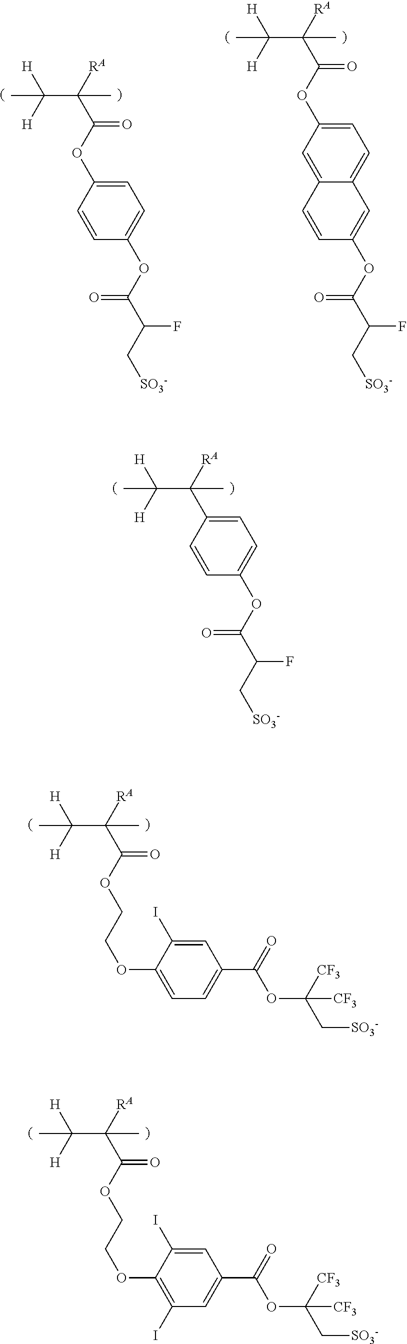

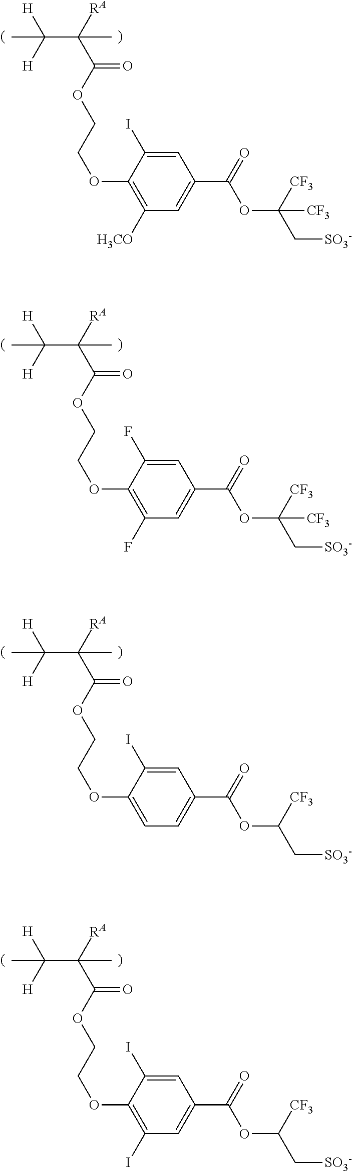

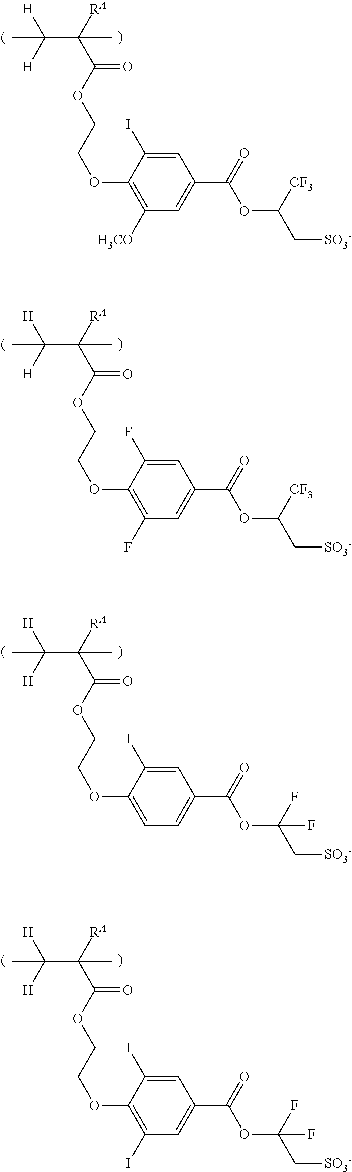

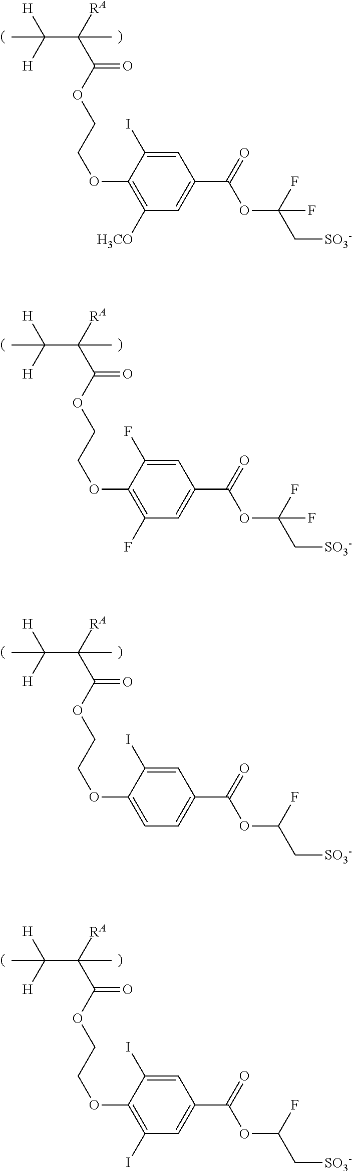

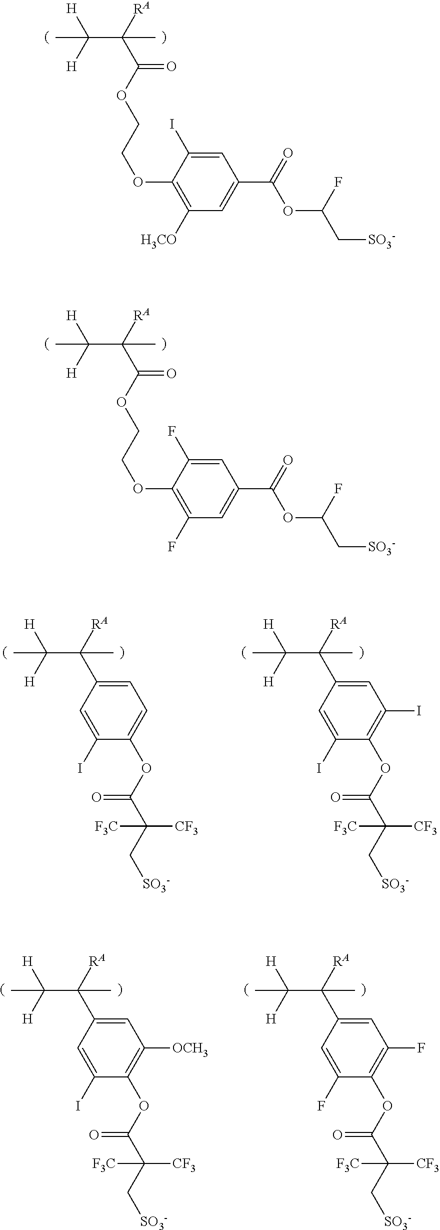

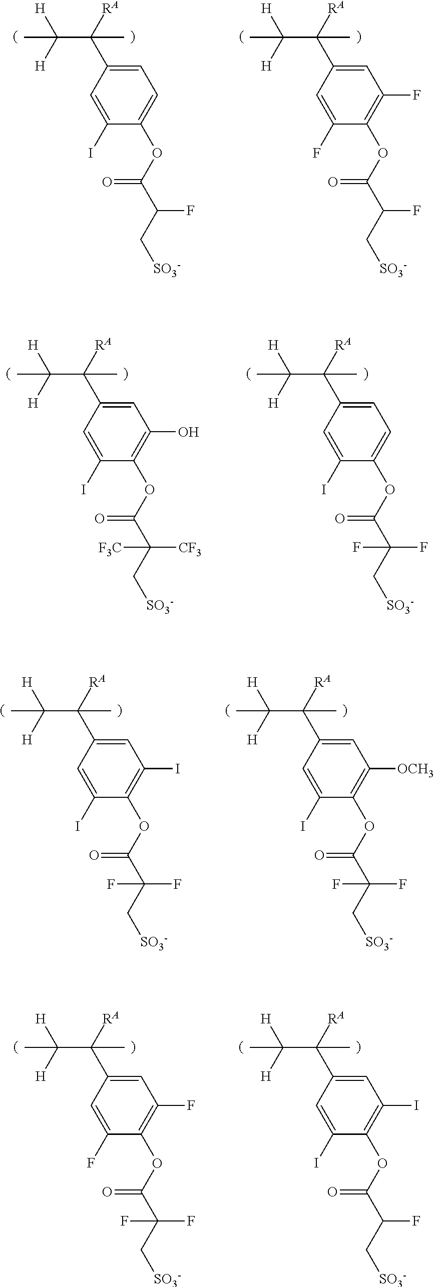

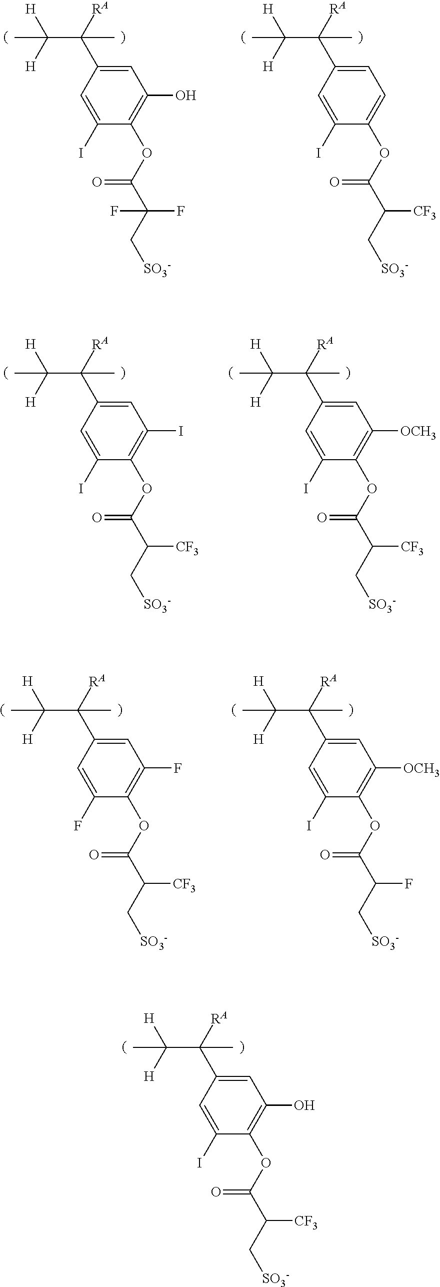

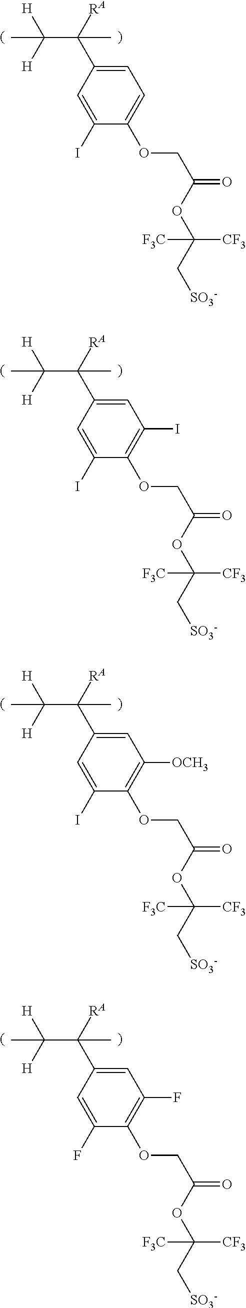

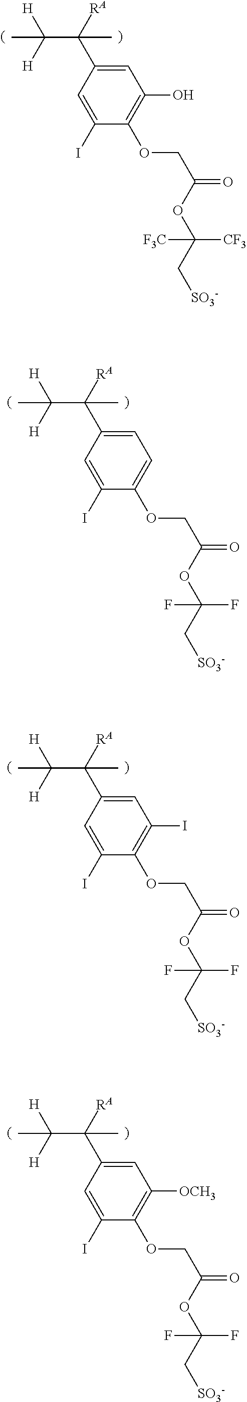

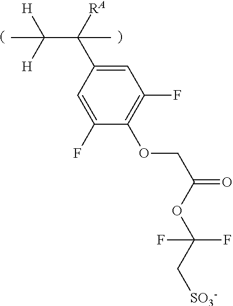

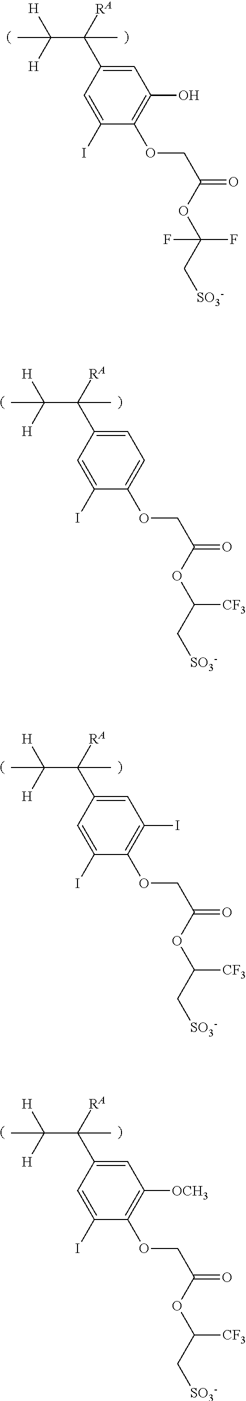

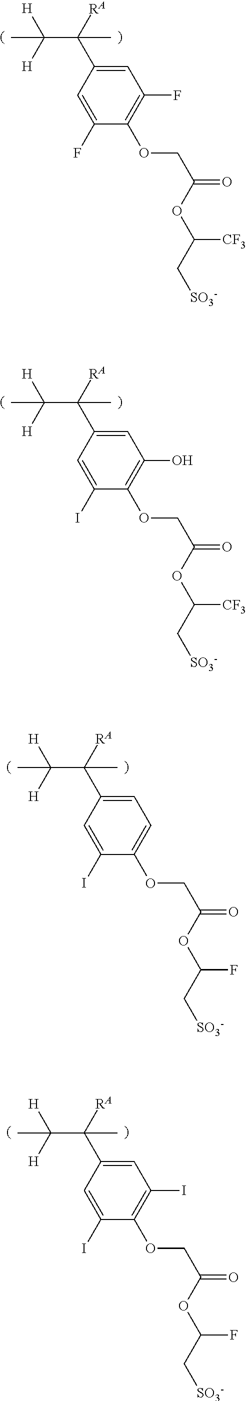

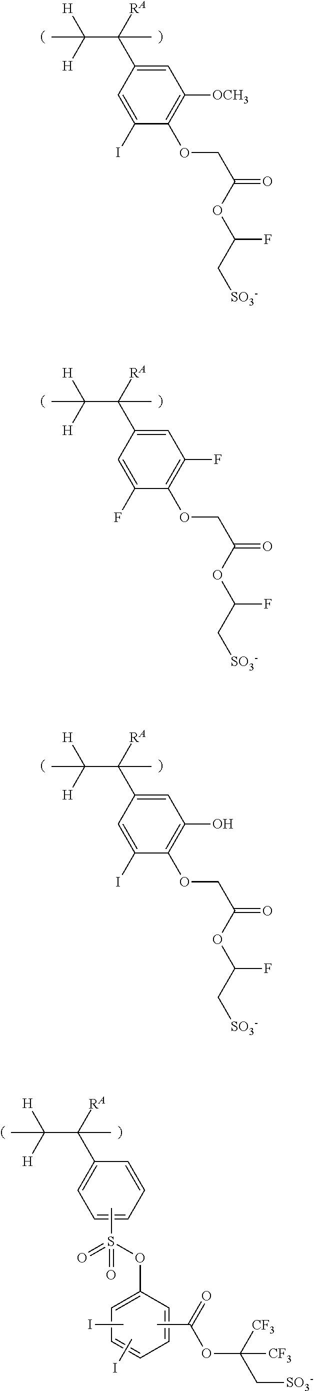

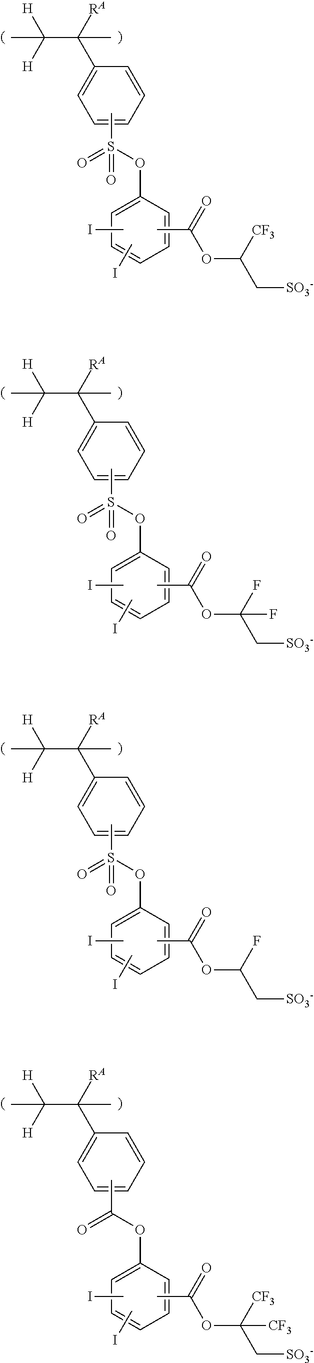

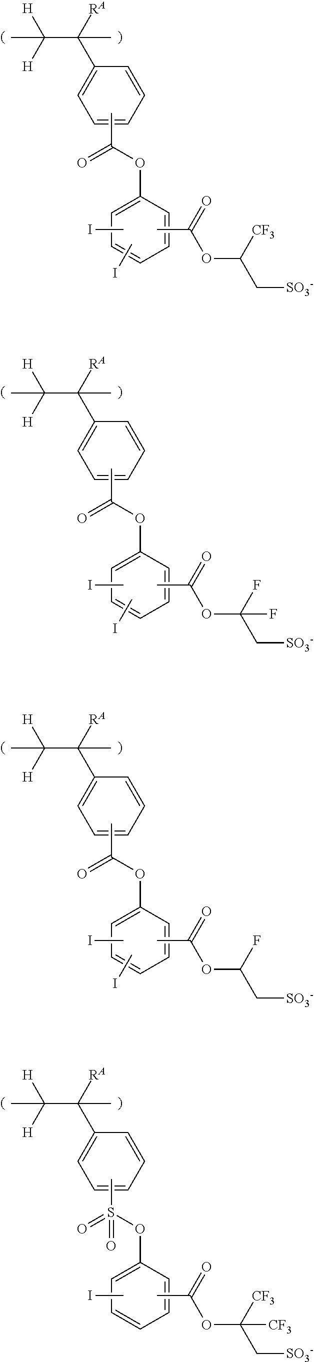

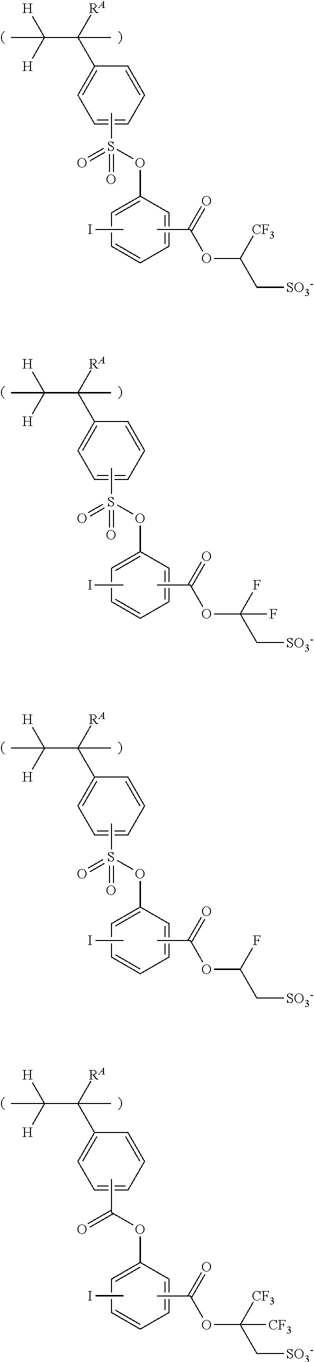

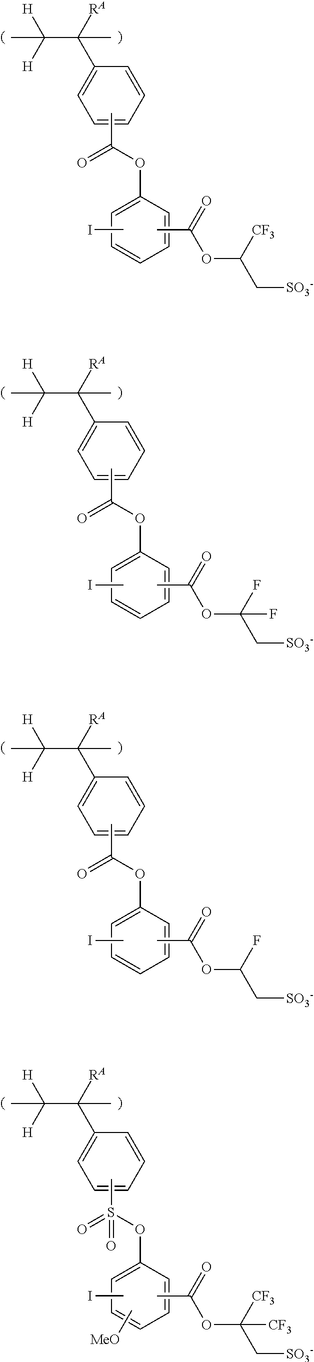

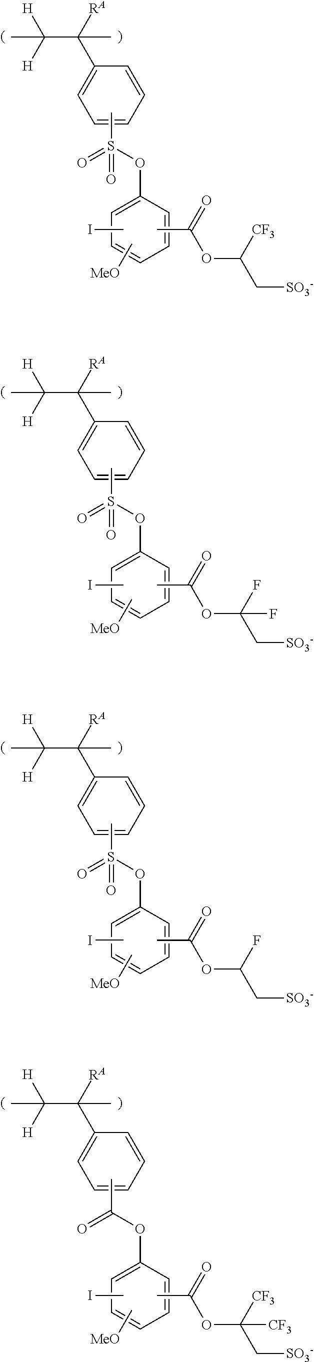

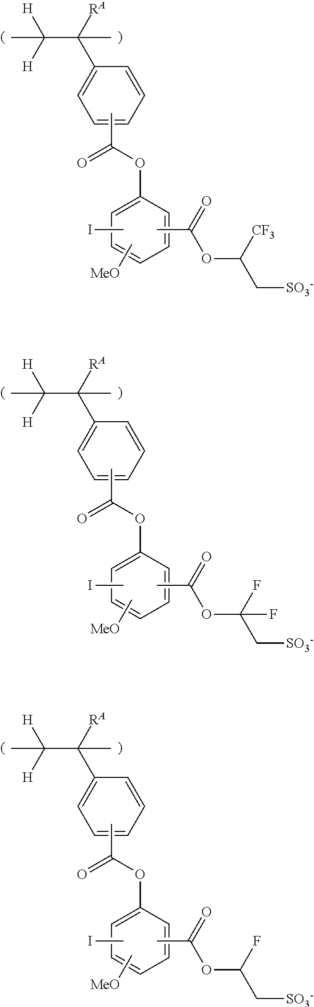

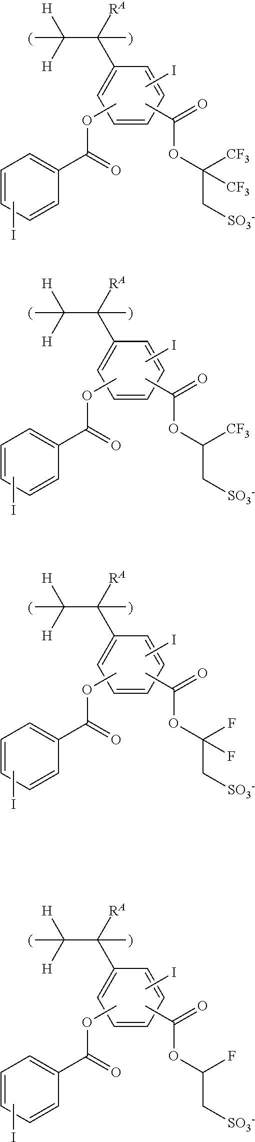

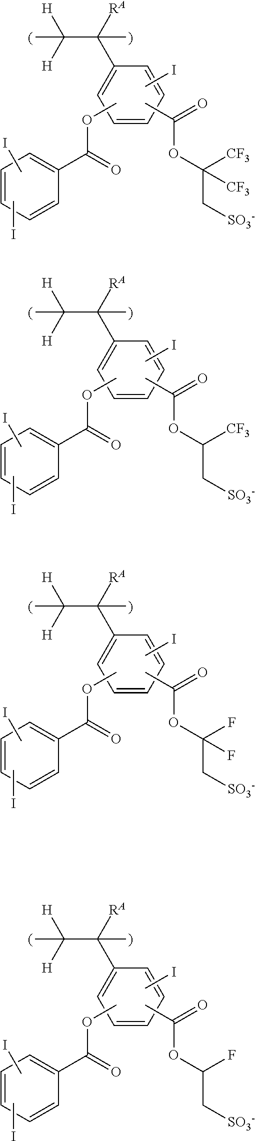

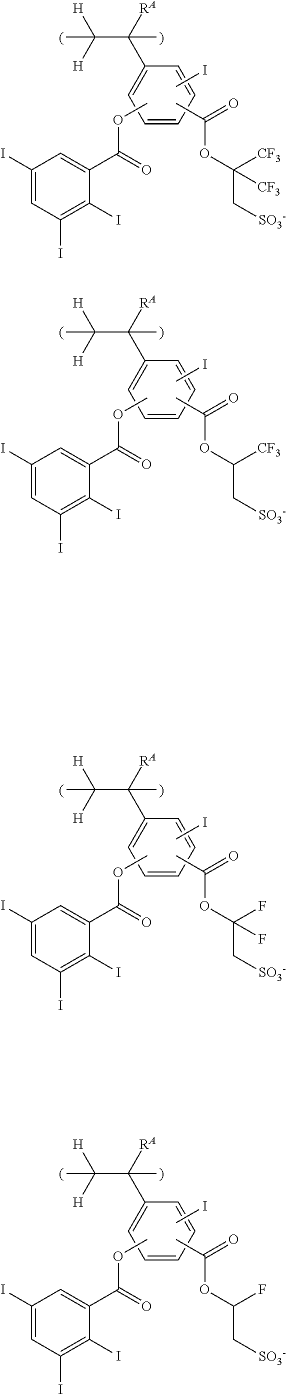

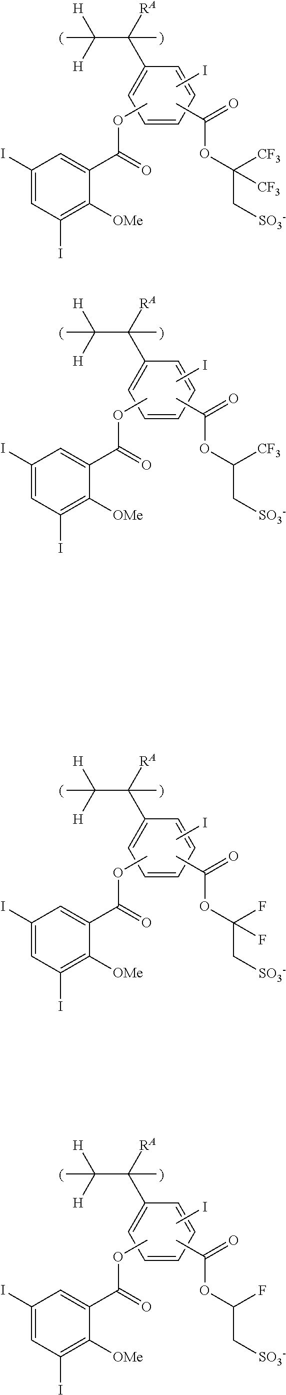

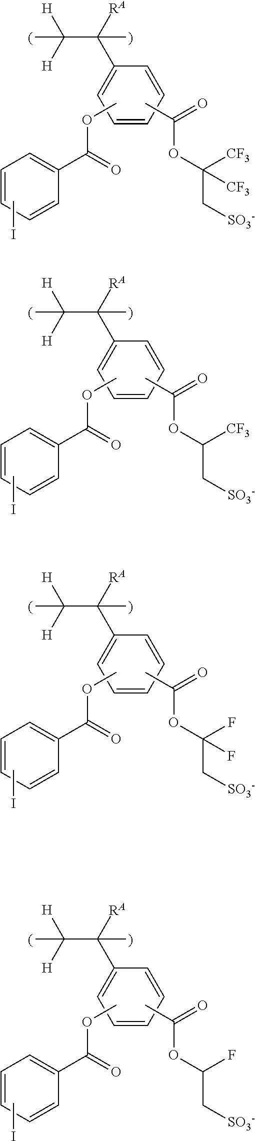

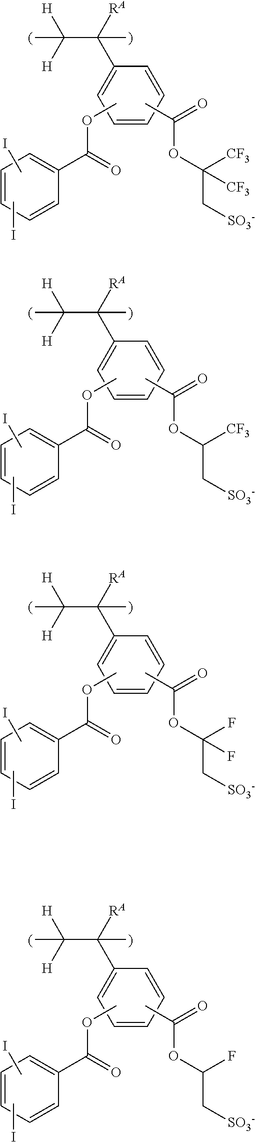

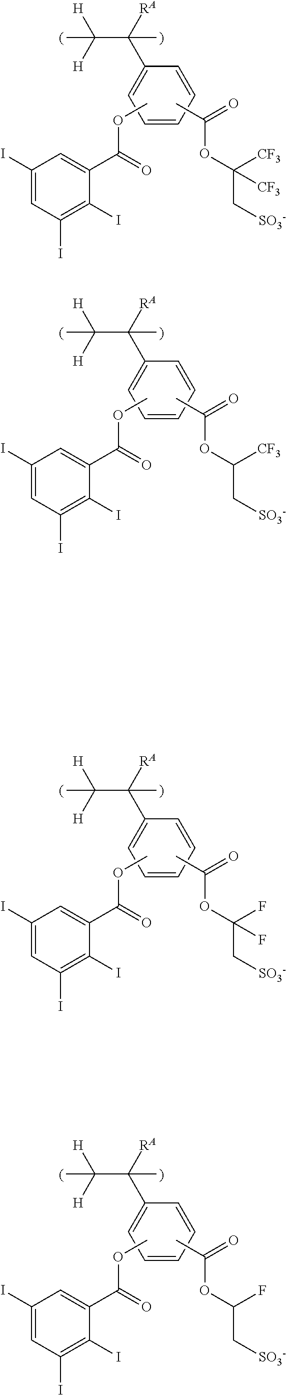

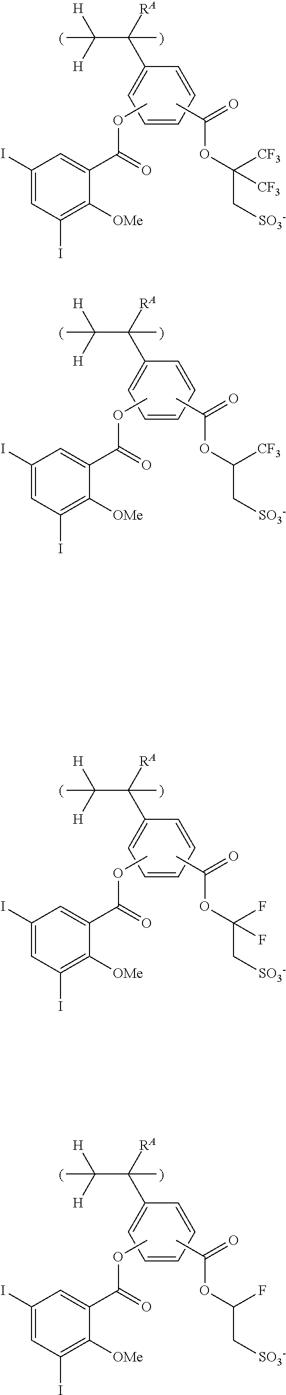

Specific examples of the anion of the repeat units c4 are shown below, but are not limited thereto. Herein R^{A} is as defined above.

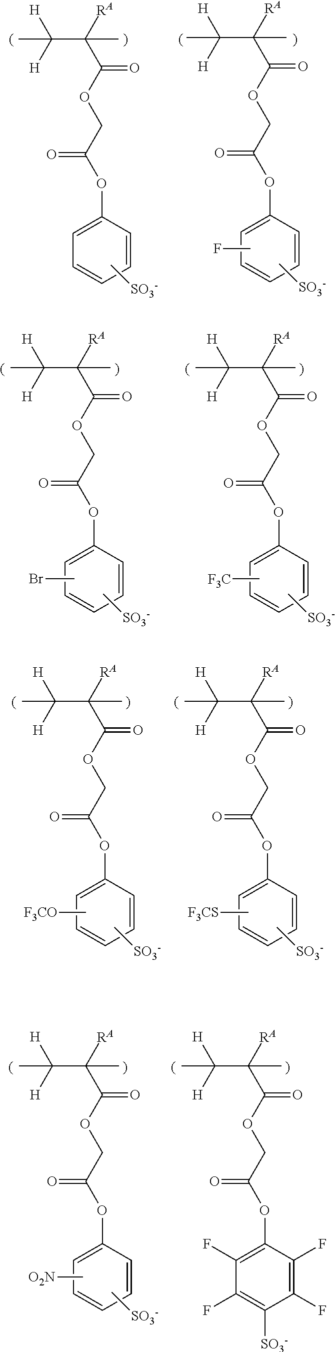

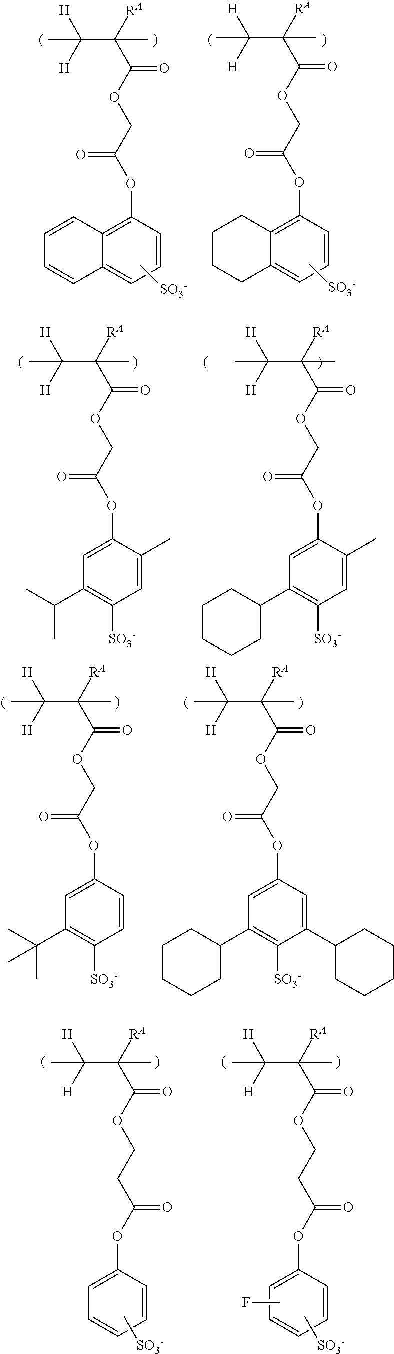

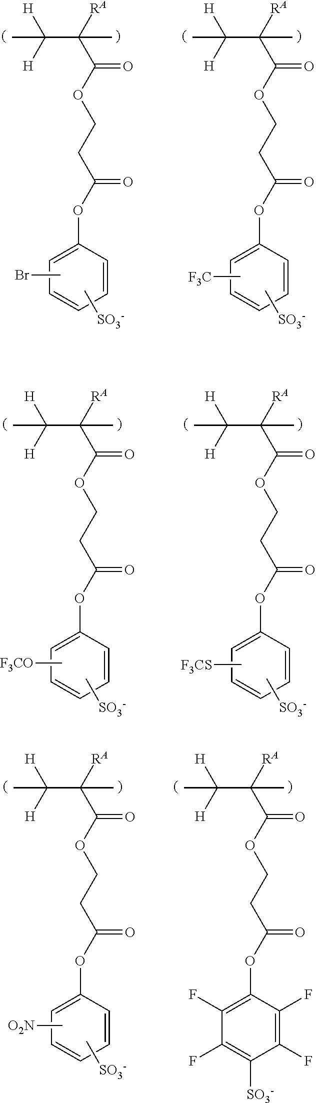

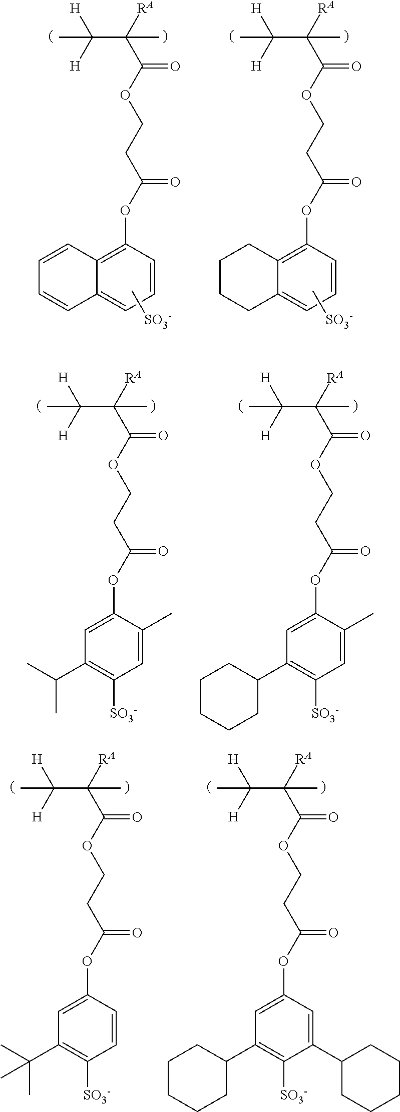

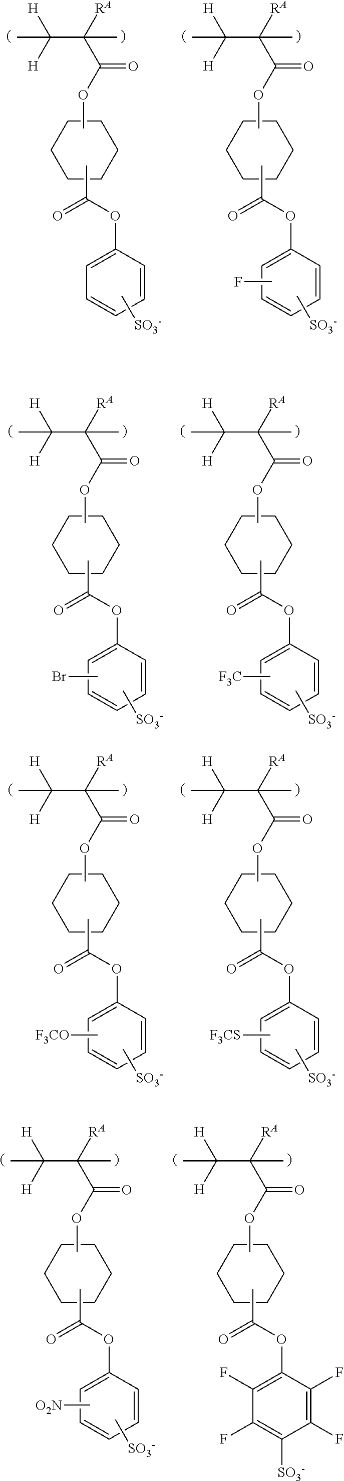

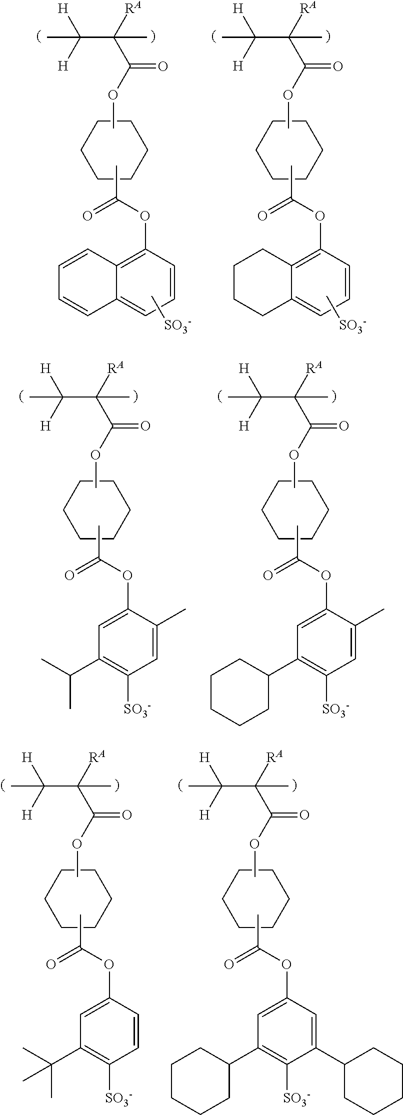

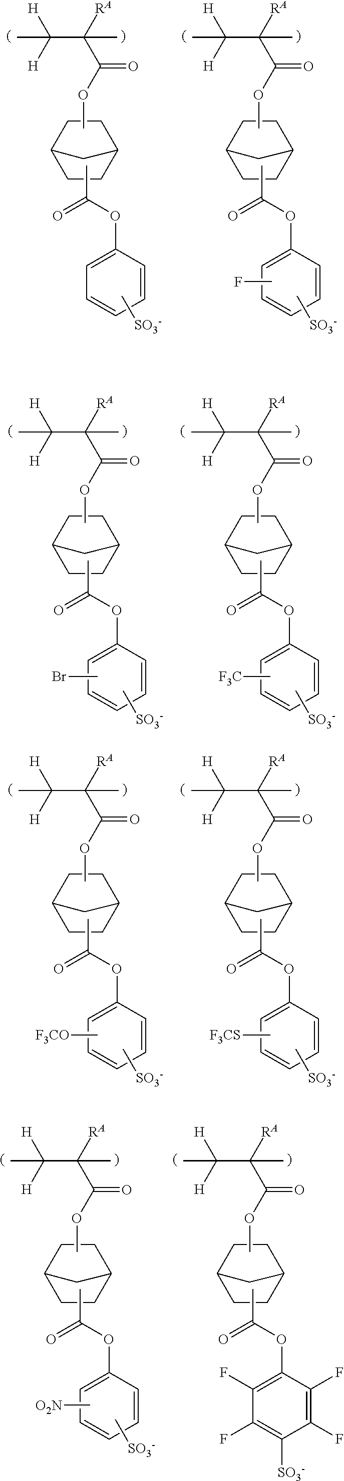

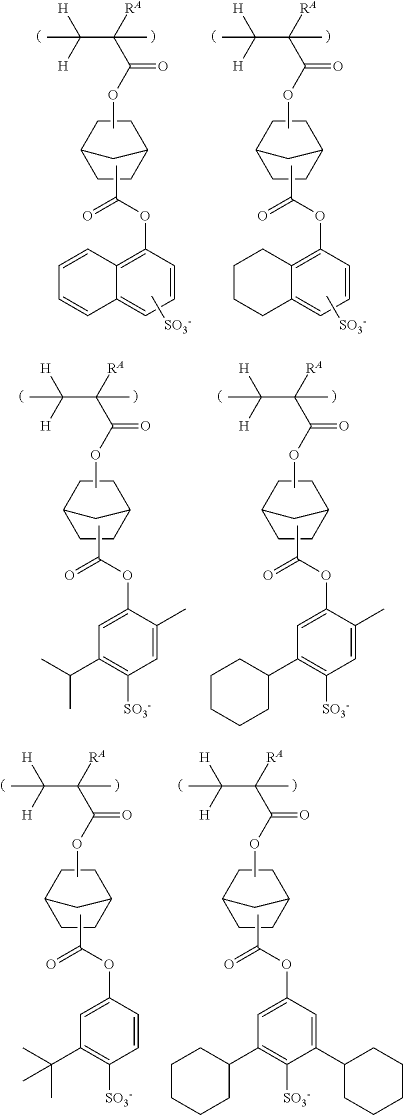

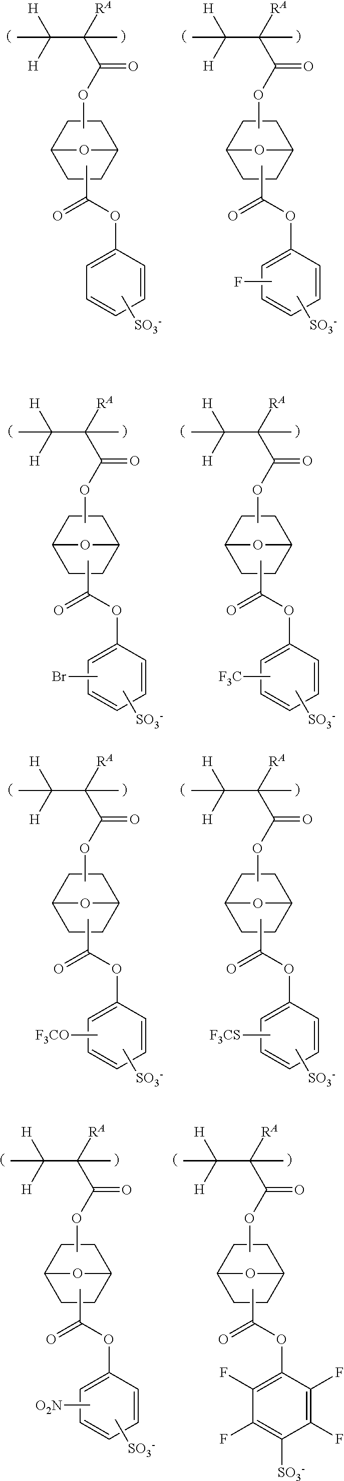

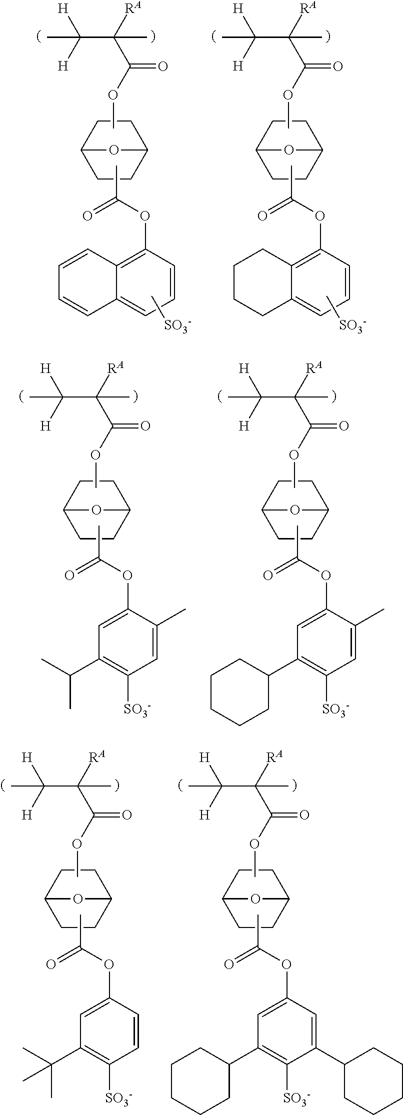

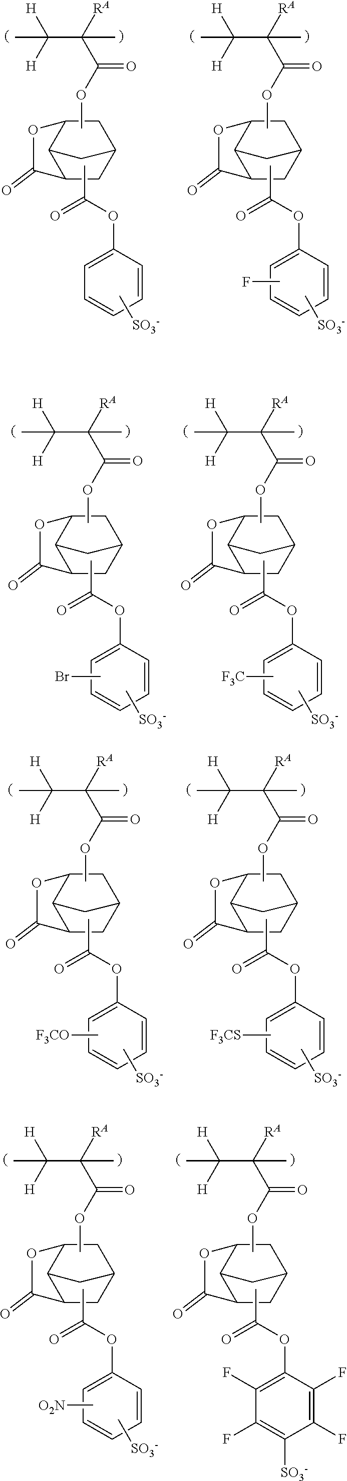

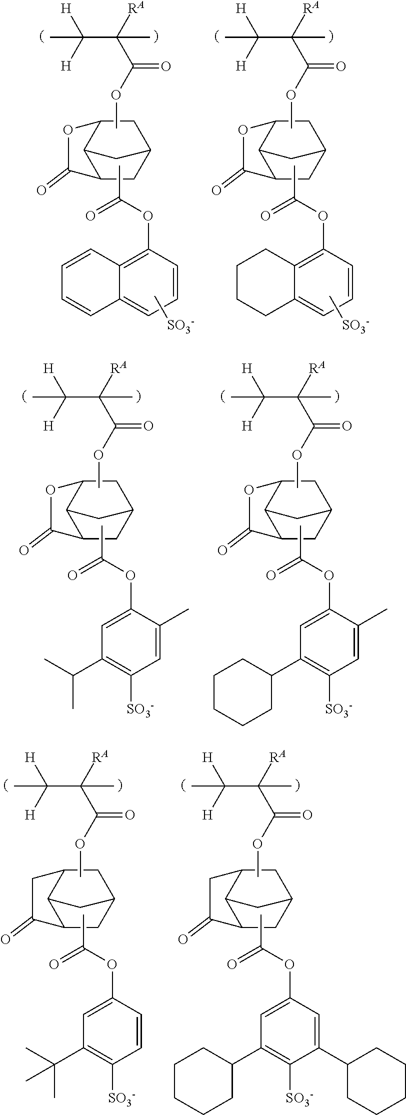

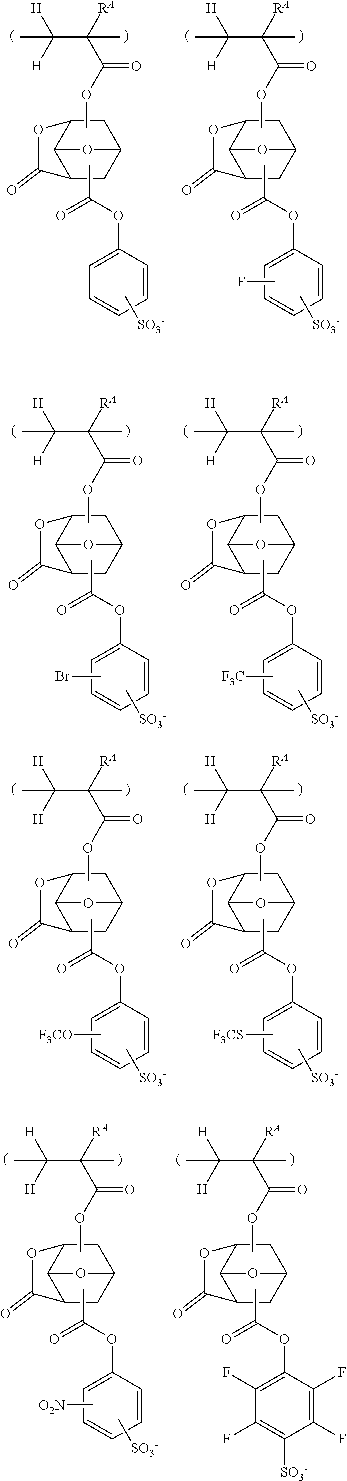

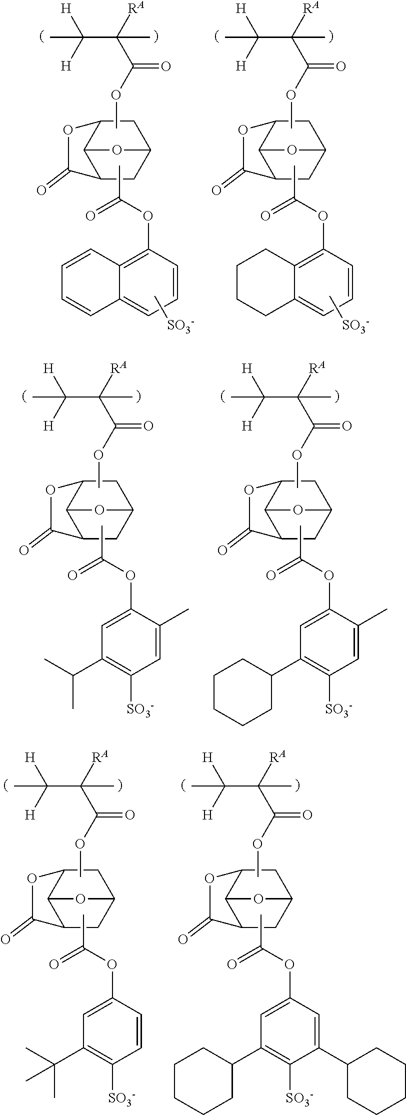

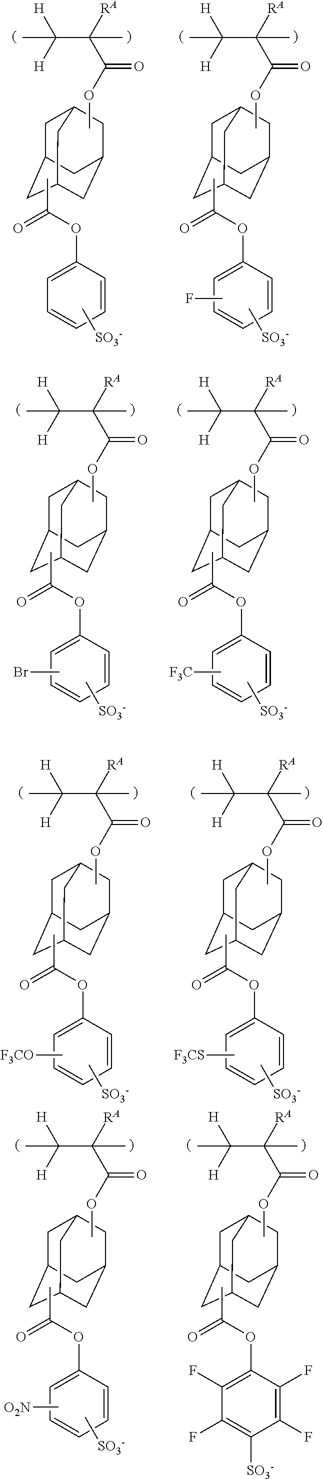

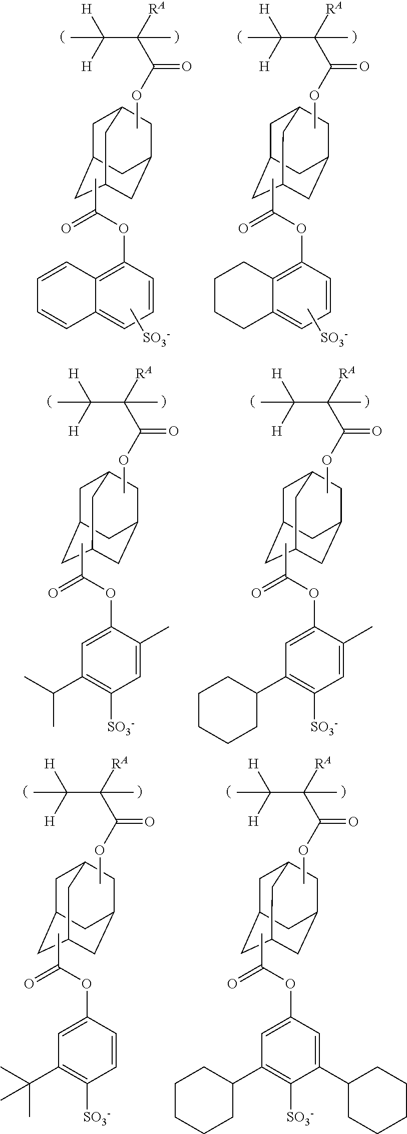

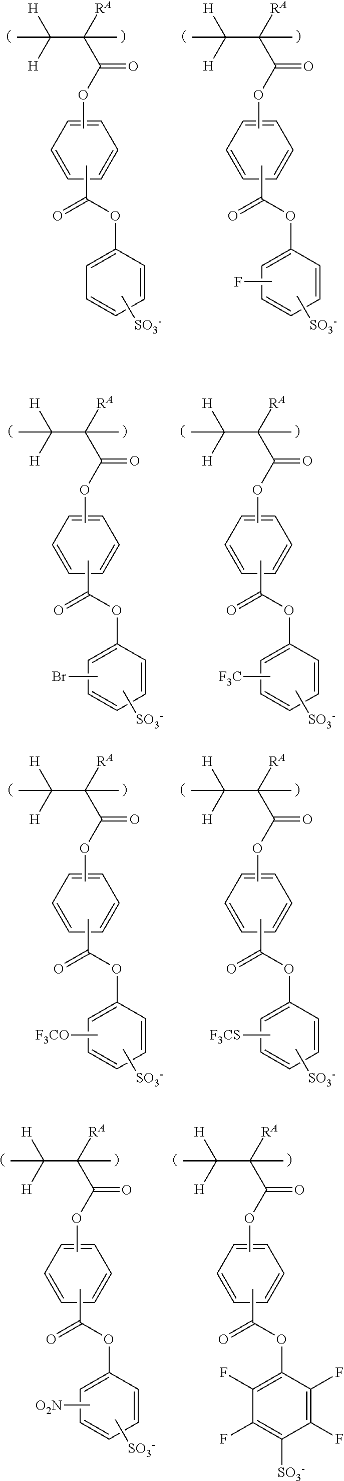

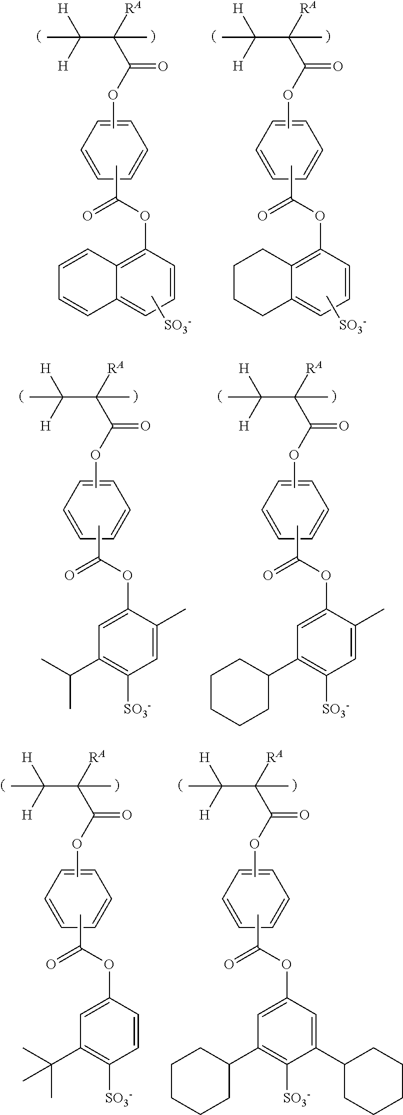

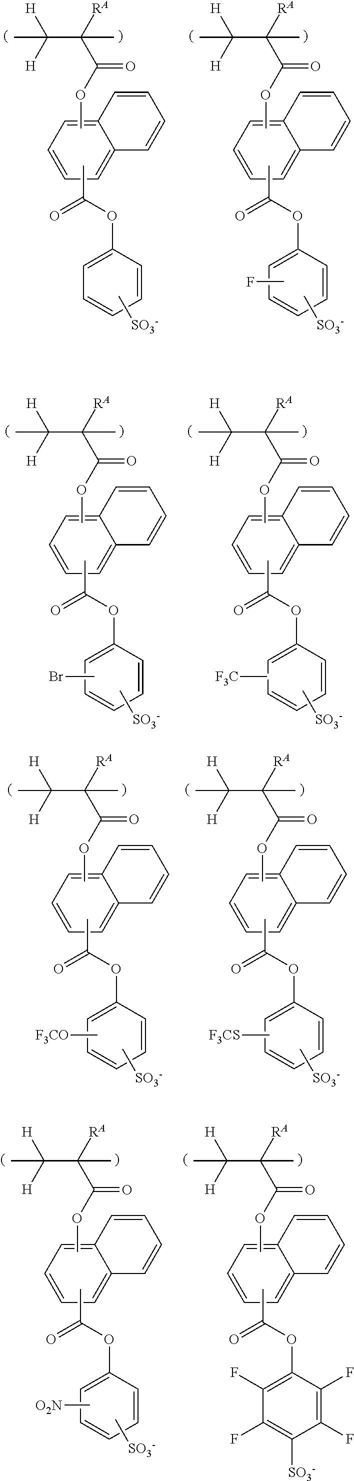

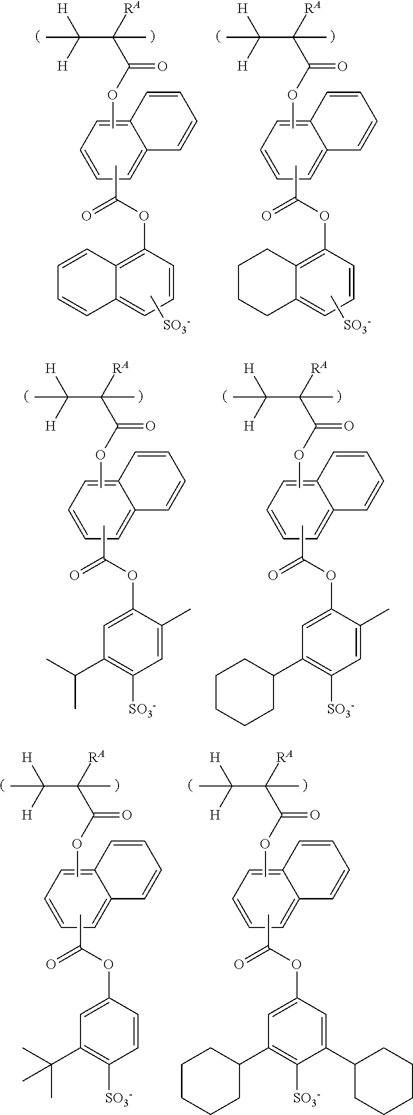

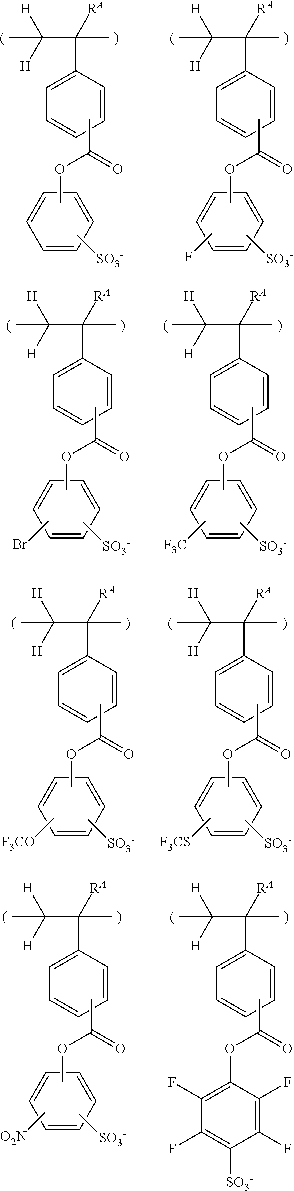

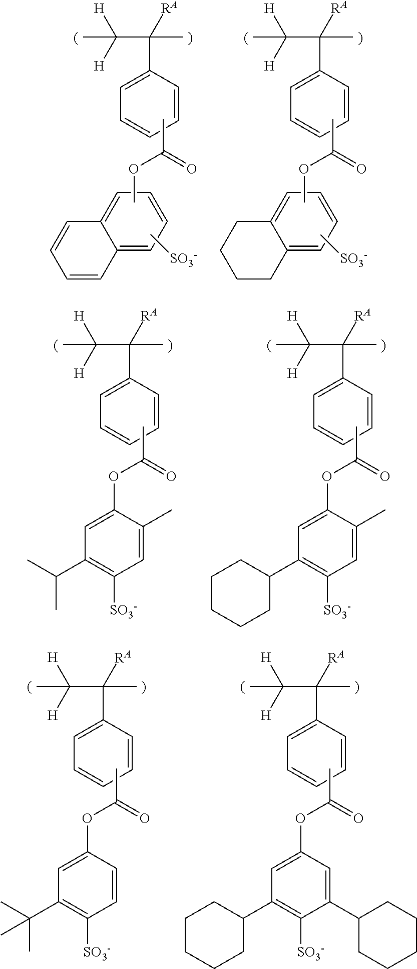

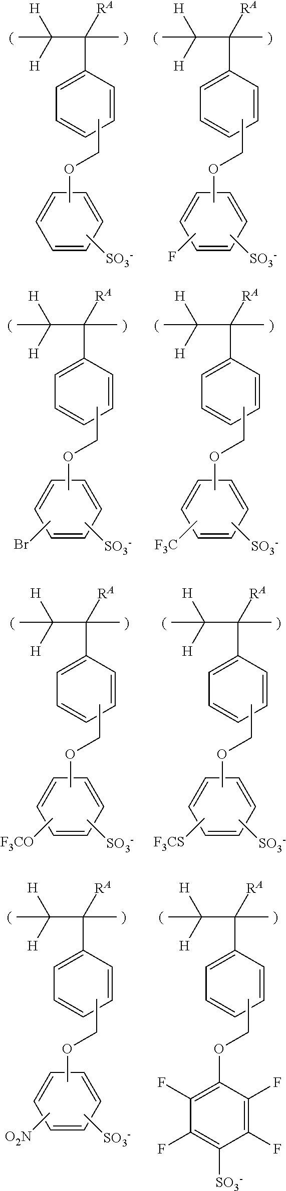

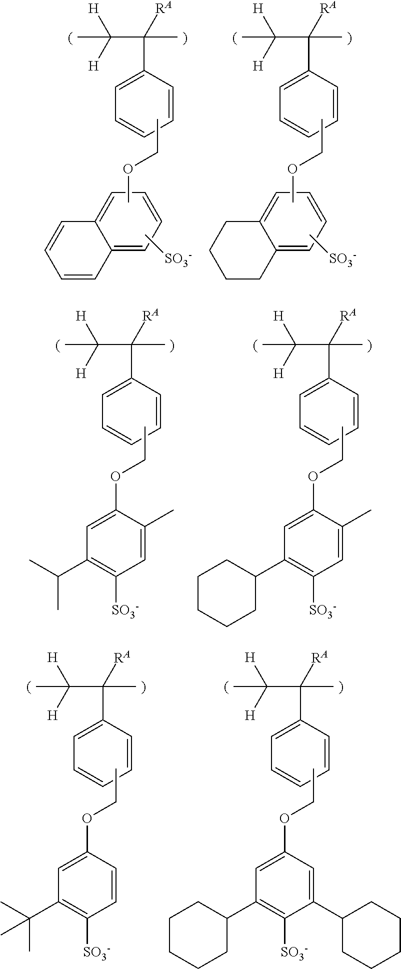

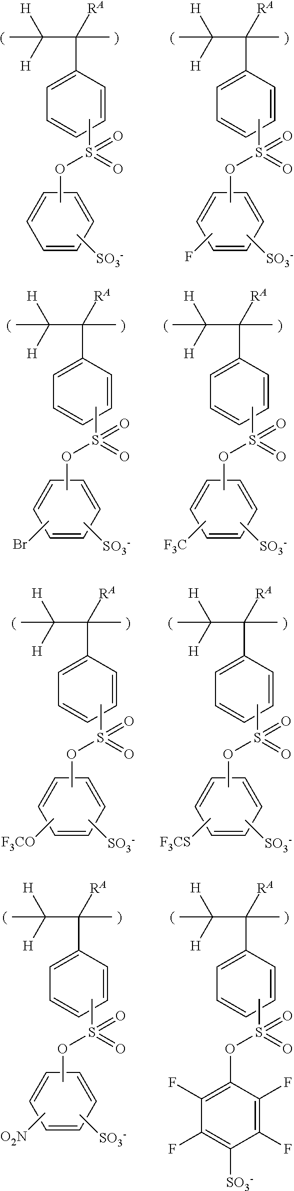

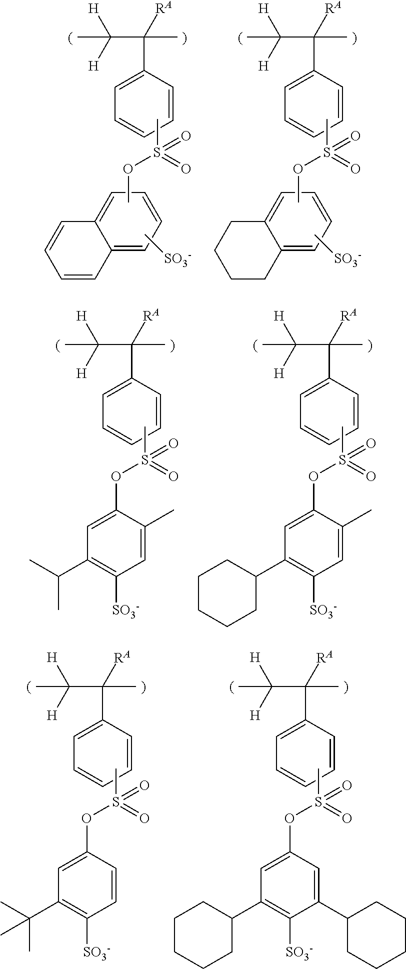

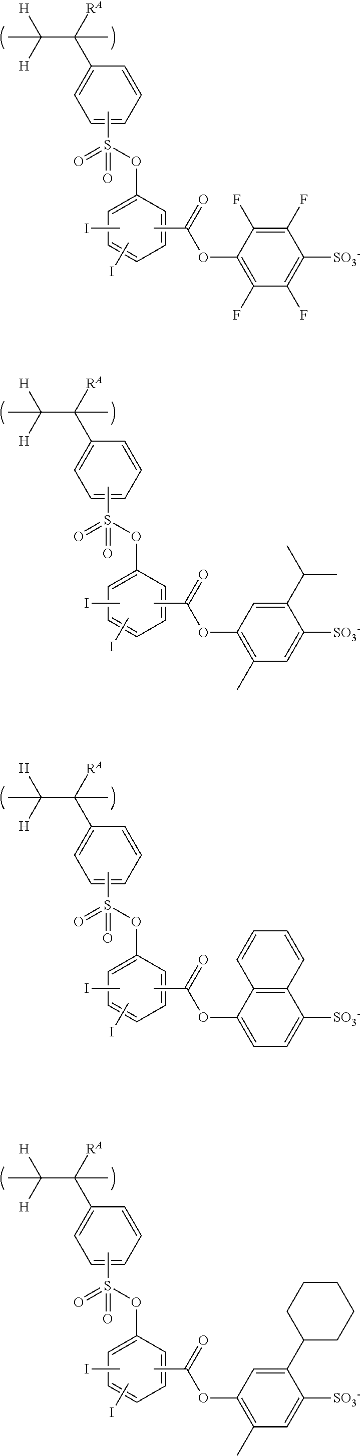

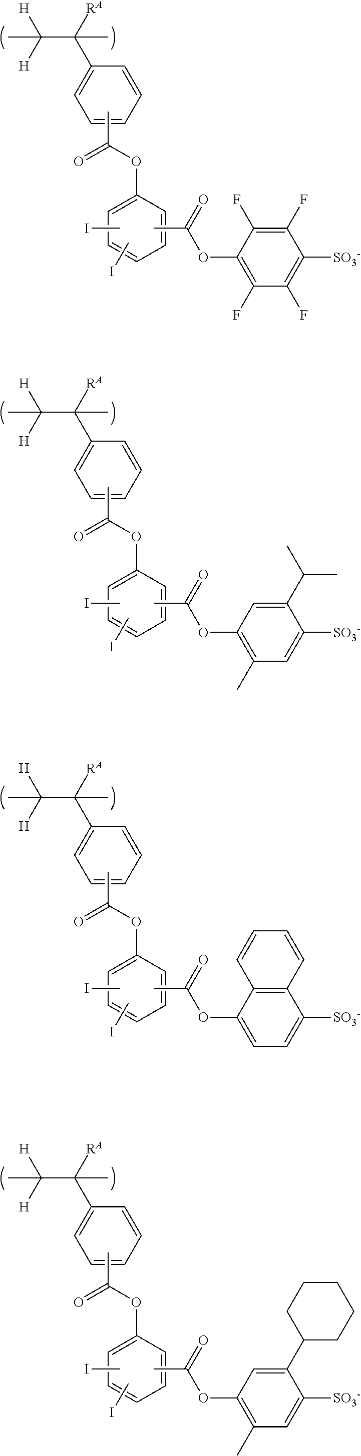

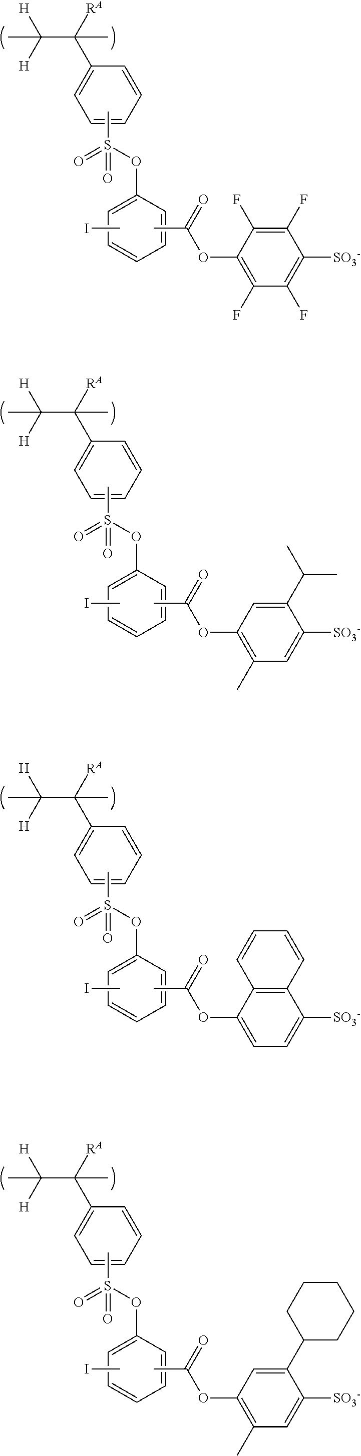

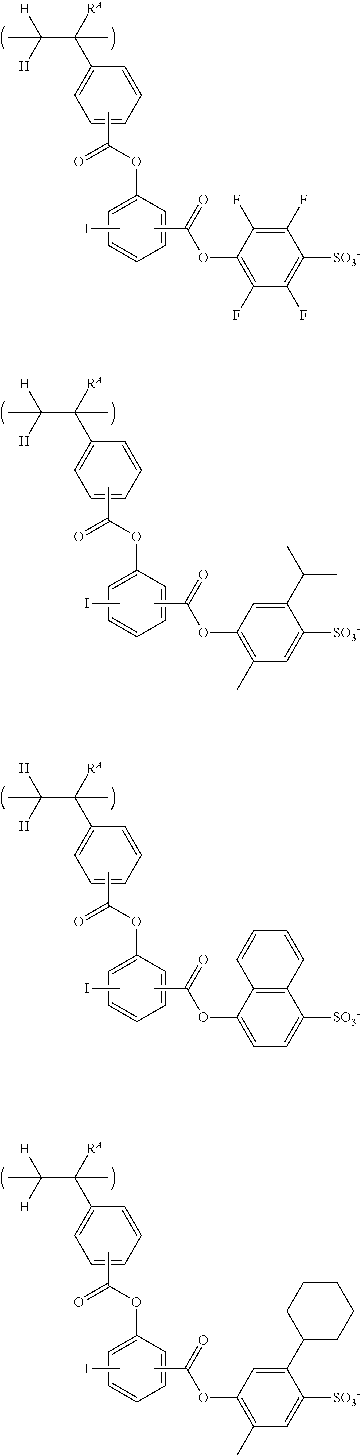

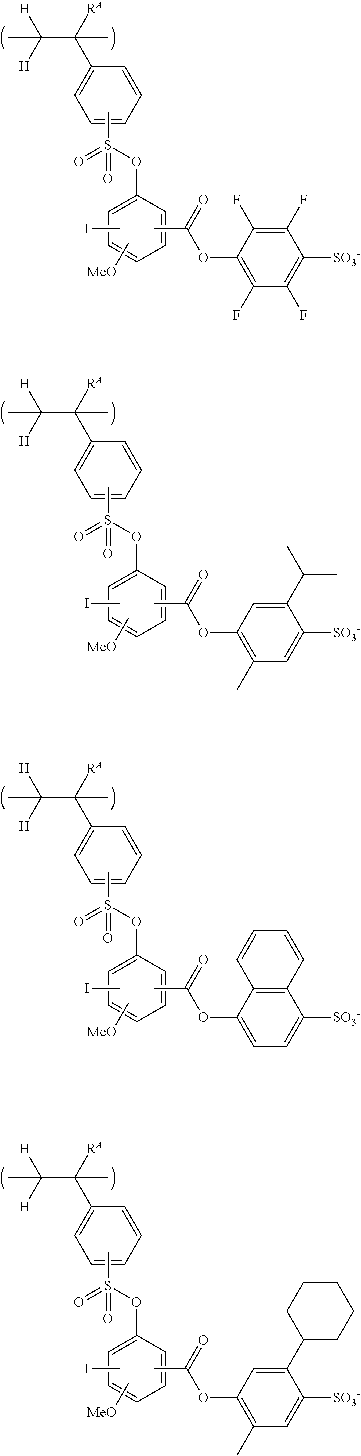

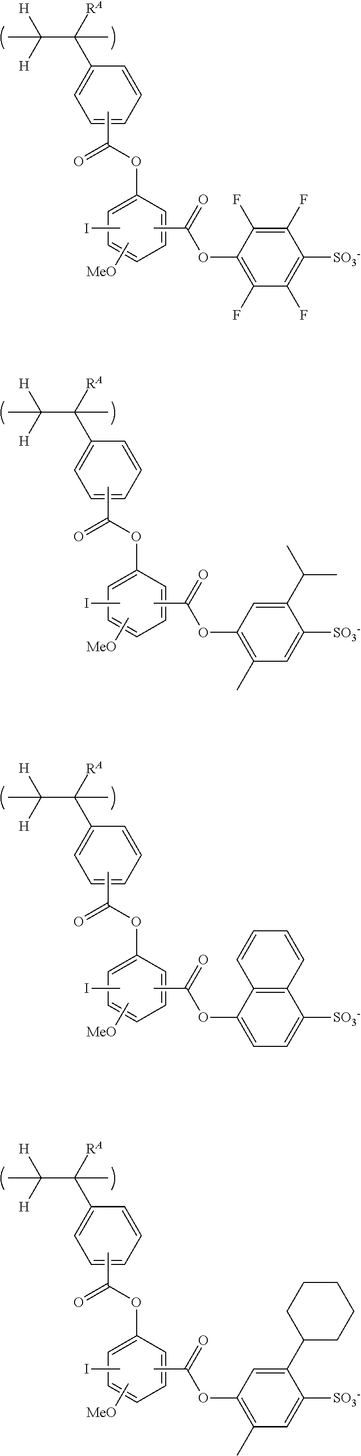

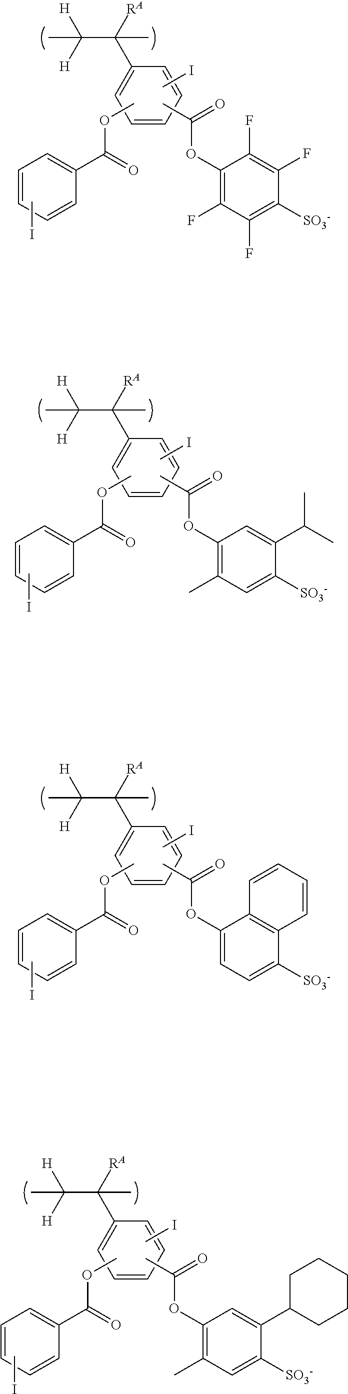

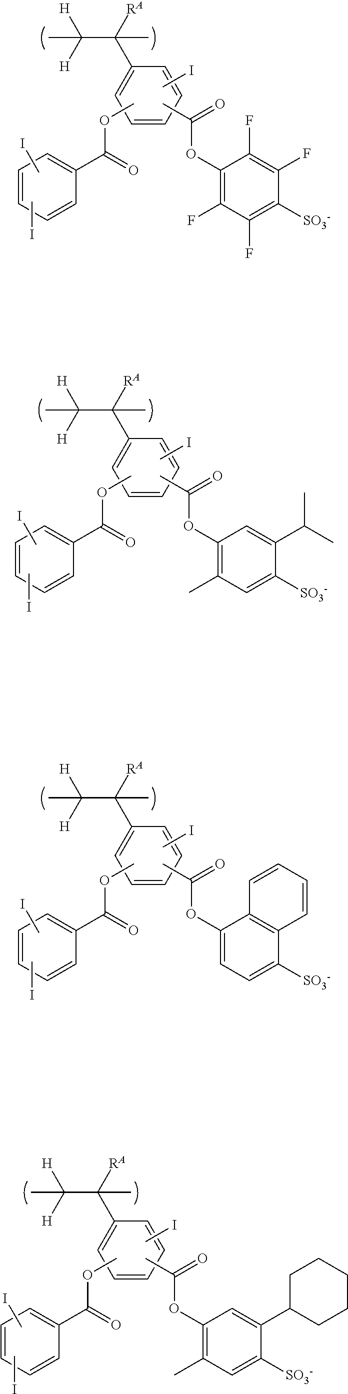

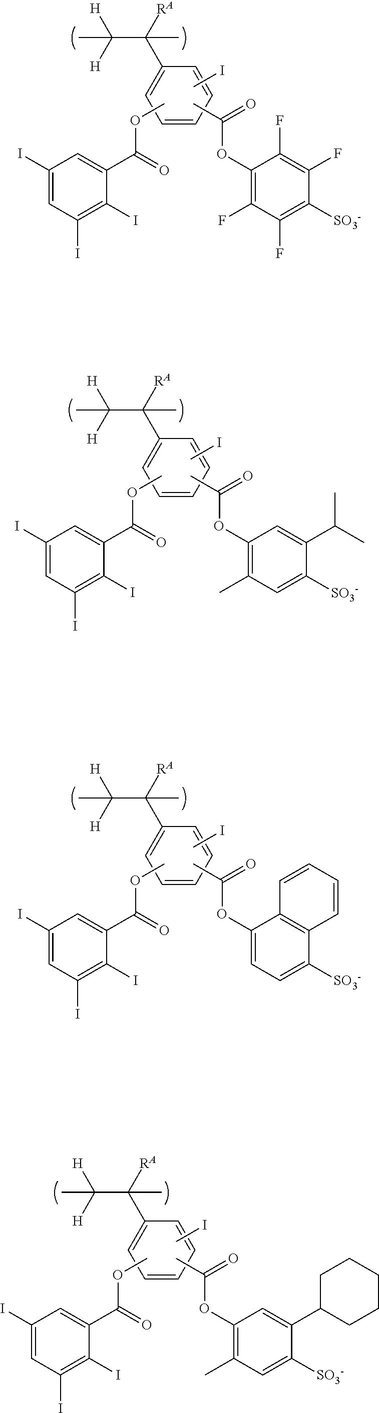

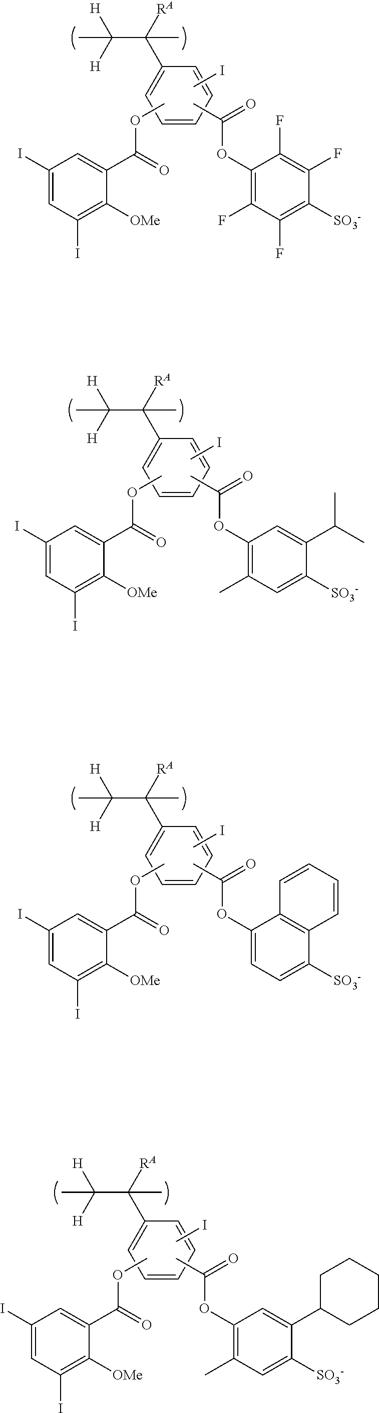

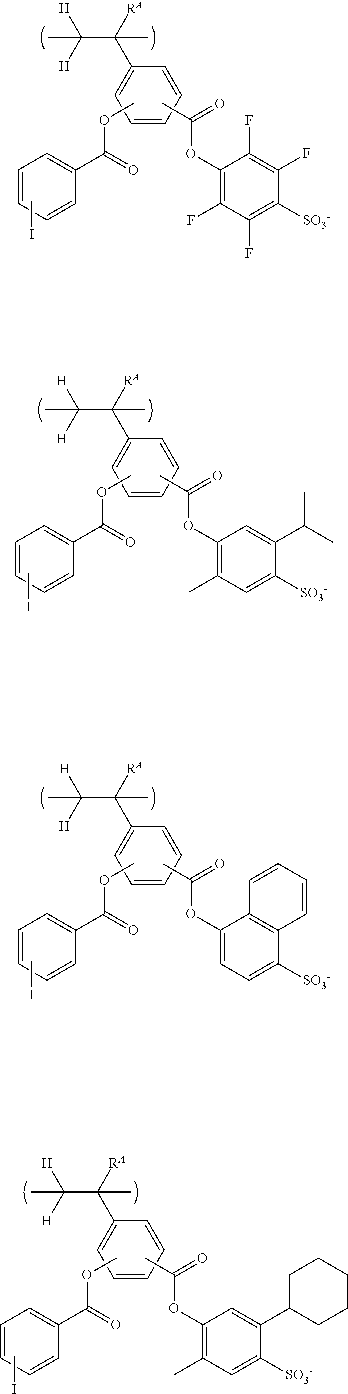

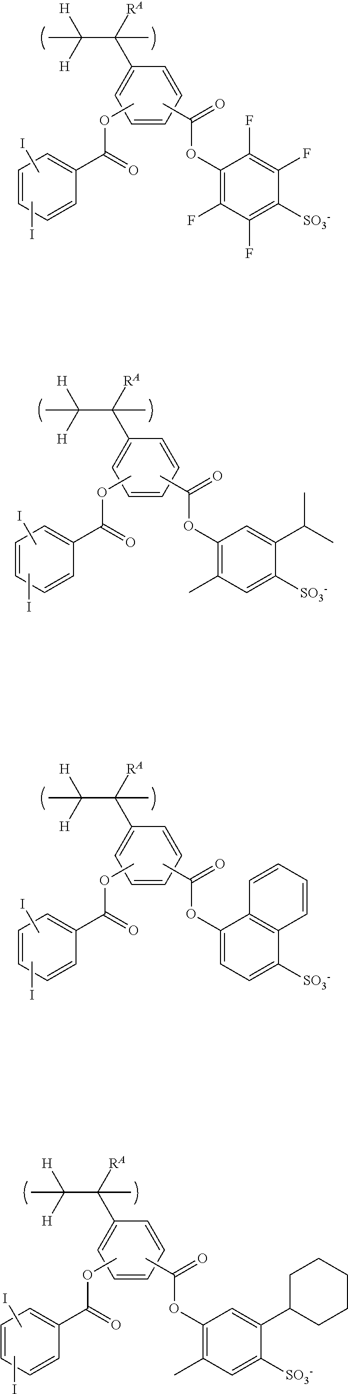

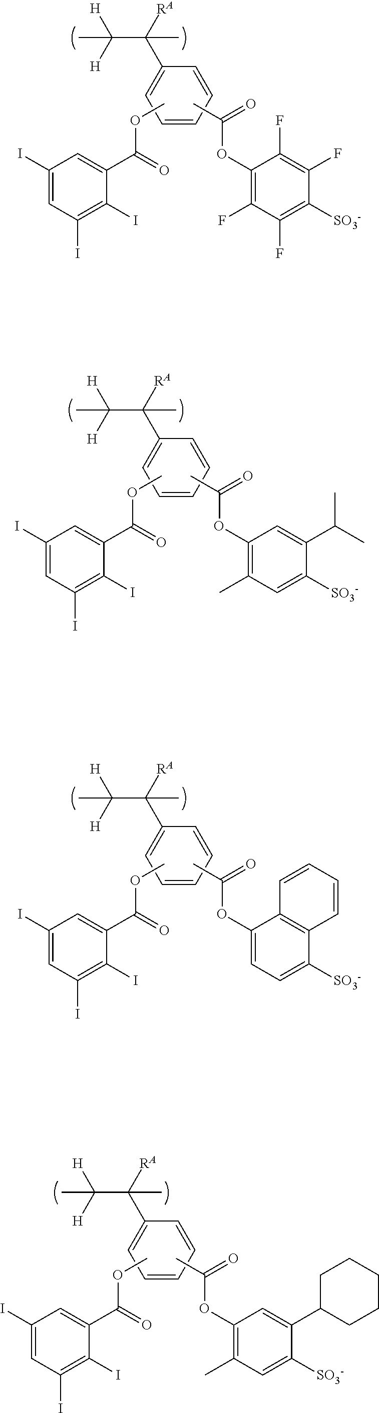

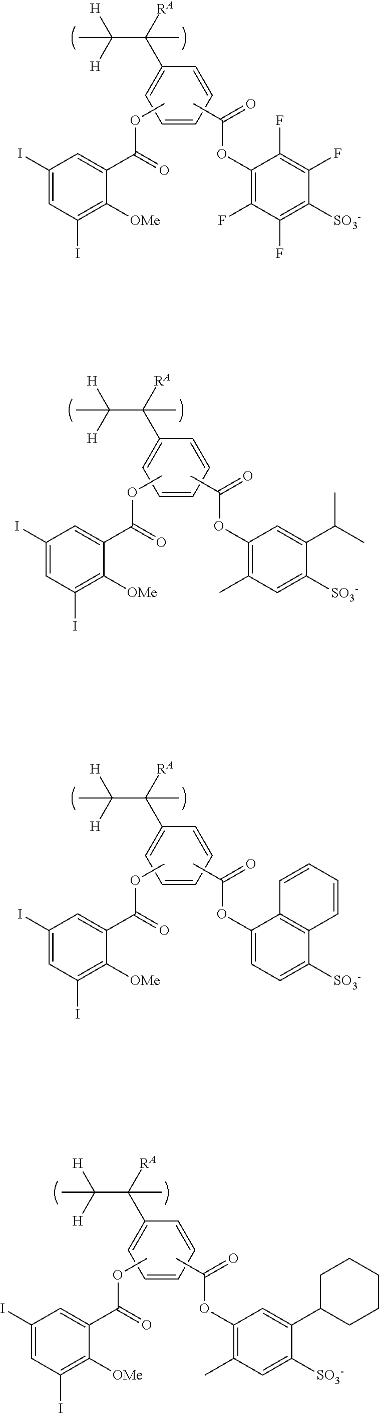

Specific examples of the anion of the repeat units c5 are shown below, but are not limited thereto. Herein R^{A} is as defined above.

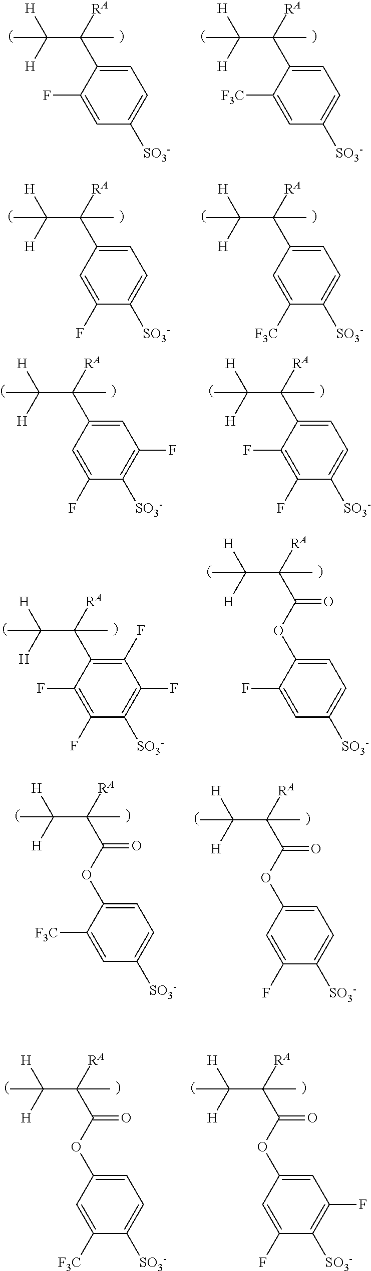

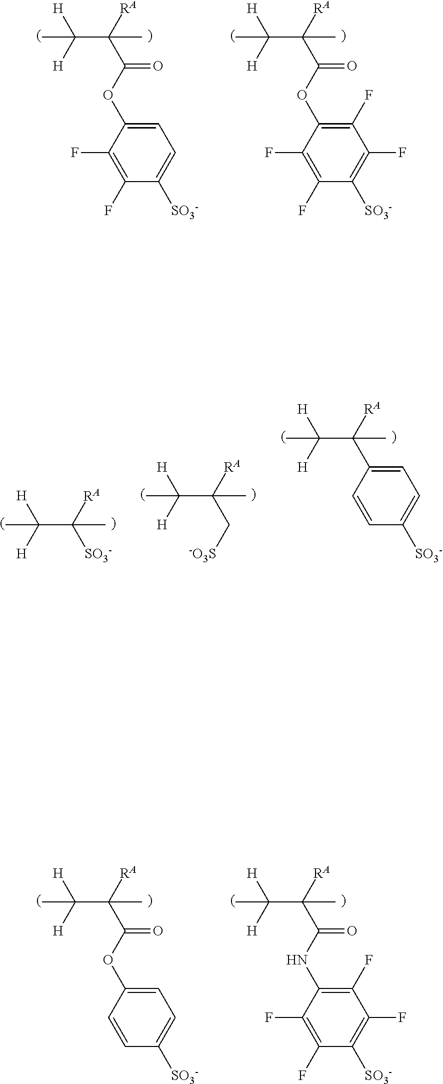

In formulae (c2) to (c5), A⁺ is an onium cation. Examples of the onium cation include a sulfonium cation, an iodonium cation, and an ammonium cation, and a sulfonium cation and an iodonium cation are preferred. Specific examples of the sulfonium cation include those having formula (Z-1), those described in paragraphs to of JP-A 2024-003744, those described in paragraphs to of JP-A 2023-169812, and those having formula (Z-3), but are not limited thereto. Specific examples of the iodonium cation include those having formula (Z-2) and those described in paragraph of JP-A 2024-000259, but are not limited thereto. Specific examples of the ammonium cation include those exemplified as the ammonium cation having the formula (am-1) described below.

Specific structures of the repeat units c1 to c5 include any combination of the anion and the cation described above.

Among the repeat units c1 to c5, the repeat units c2 to c5 are preferred from the viewpoint of controlling acid diffusion, the repeat units c2, c4, and c5 are more preferred from the viewpoint of acid strength of the generated acid, and the repeat units c2 are still more preferred from the viewpoint of solvent solubility.

The base polymer may comprise repeat units having a structure in which a hydroxy group is protected with an acid labile group (hereinafter, also referred to as repeat units d). The repeat unit d is not particularly limited as long as the unit has one or two or more structures having a hydroxy group protected with a protective group such that the protective group is decomposed to generate the hydroxy group under the action of acid. Repeat units having the formula (d1) are preferred.

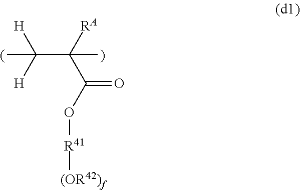

In formula (d1), R^{A} is as defined above. R⁴¹ is a C₁-C₃₀ (f+1)-valent hydrocarbon group which may contain a heteroatom. R⁴² is an acid labile group. f is 1, 2, 3, or 4.

In formula (d1), the acid labile group R⁴² is deprotected under the action of acid so that a hydroxy group is generated. The structure of R⁴² is not particularly limited. An acetal structure, a ketal structure, an alkoxycarbonyl group, and an alkoxymethyl group having the formula (d2) are preferred, and an alkoxymethyl group having the formula (d2) is particularly preferred.

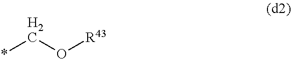

Herein * designates a point of attachment. R⁴³ is a C₁-C₁₅ hydrocarbyl group.

Specific examples of the acid labile group R⁴², the alkoxymethyl group having formula (d2), and the repeat units d are as exemplified for the repeat units d in JP-A 2020-111564.

The base polymer may comprise repeat units e derived from indene, benzofuran, benzothiophene, acenaphthylene, chromone, coumarin, norbornadiene, and derivatives thereof. Specific examples of the monomer from which repeat units e are derived are shown below, but are not limited thereto.

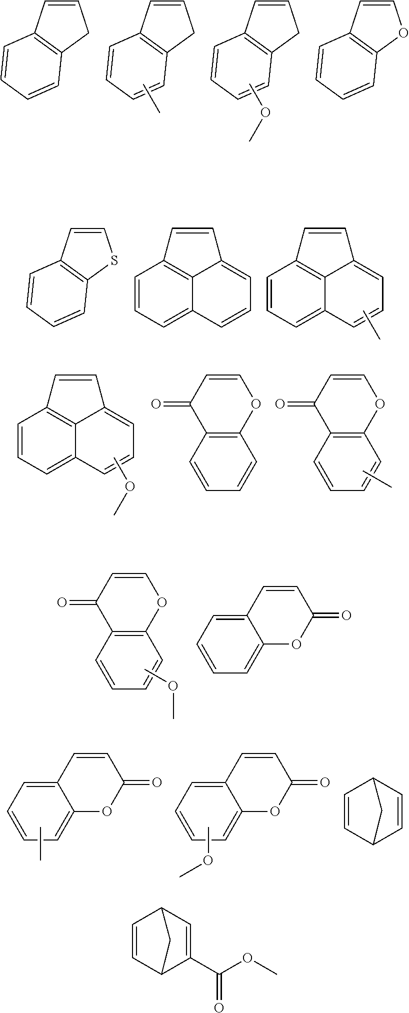

The base polymer may comprise repeat units f derived from indane, vinylpyridine, or vinylcarbazole.

The base polymer for the resist composition essentially comprises any of the repeat units a1 to a3 containing an acid labile group. In this case, in the base polymer, repeat units a1, a2, a3, b1, b2, c1 to c5, d, e, and f are incorporated in a content ratio of preferably 0<a1≤0.8, 0≤a2≤0.8, 0≤a3≤0.6, 0≤b1≤0.6, 0≤b2≤0.6, 0≤c1≤0.4, 0≤c2≤0.4, 0≤c3≤0.4, 0≤c4≤0.4, 0≤c5≤0.4, 0≤d≤0.5, 0≤e≤0.3, and 0≤f≤0.3, and more preferably 0<a1≤0.7, 0≤a2≤0.7, 0≤a3≤0.5, 0≤b1≤0.5, 0≤b2≤0.5, 0≤c1≤0.3, 0≤c2≤0.3, 0≤c3≤0.3, 0≤c4≤0.3, 0≤c5≤0.3, 0≤d≤0.3, 0≤e≤0.3, and 0≤f≤0.3. Provided that a1+a2+a3+b1+b2+c1+c2+c3+c4+c5+d+e+f≤1.0.

A weight average molecular weight (Mw) of the polymer is preferably 1,000 to 500,000, and more preferably 3,000 to 100,000. When the Mw is in the range, sufficient etching resistance is obtained, and there is no possibility of resolution decline due to a failure to ensure a difference in dissolution rate before and after exposure. It is noted that the Mw as used in the present invention is measured by gel permeation chromatography (GPC) versus polystyrene standards using THF or N,N-dimethylformamide (DMF) as a solvent.

The influence of Mw/Mn becomes stronger as the pattern rule becomes finer. Therefore, the polymer should preferably have a narrow dispersity (Mw/Mn) of 1.0 to 2.0 in order to provide a resist composition suitable for micropatterning to a small feature size. Mw/Mn in the above range ensures that the contents of lower and higher molecular weight polymer fractions are low and eliminates a possibility that foreign matter is left on the pattern or the pattern profile is degraded.

The polymer may be synthesized, for example, by dissolving a monomer or monomers corresponding to the above-mentioned repeat units in an organic solvent, adding a radical polymerization initiator, and heating for polymerization.

Specific examples of the organic solvent which can be used for polymerization include toluene, benzene, THF, diethyl ether, dioxane, cyclohexane, cyclopentane, methyl ethyl ketone (MEK), propylene glycol monomethyl ether acetate (PGMEA), and γ-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The initiator is preferably added in an amount of 0.01 to 25 mol % based on the total of monomers to be polymerized. The reaction temperature is preferably 50 to 150° C., and more preferably 60 to 100° C. The reaction time is preferably 2 to 24 hours, and more preferably 2 to 12 hours from the viewpoint of production efficiency.

The polymerization initiator may be fed to the reactor either by adding the initiator to the monomer solution and feeding the solution to the reactor, or by dissolving the initiator in a solvent to form an initiator solution and feeding the initiator solution and the monomer solution independently to the reactor. Because of a possibility that in the standby duration, the initiator generates a radical which triggers polymerization reaction to form an ultra high molecular-weight polymer, it is preferred from the standpoint of quality control to prepare the monomer solution and the initiator solution separately and add them dropwise. The acid labile group that has been incorporated in the monomer may be kept as such, or polymerization may be followed by protection or partial protection. During the polymer synthesis, any known chain transfer agent such as dodecyl mercaptan or 2-mercaptoethanol may be added for molecular weight control purpose. The amount of chain transfer agent added is preferably 0.01 to 20 mol % based on the total of monomers.

When a hydroxy-containing monomer is copolymerized, the hydroxy group is substituted by an acetal group which is susceptible to deprotection with acid, typically an ethoxyethoxy group, prior to polymerization, and the polymerization is followed by deprotection with weak acid and water. Alternatively, the hydroxy group is substituted by an acetyl group, a formyl group, or a pivaloyl group group prior to polymerization, and the polymerization is followed by alkaline hydrolysis.

When hydroxystyrene or hydroxyvinylnaphthalene is copolymerized, one method is dissolving hydroxystyrene or hydroxyvinylnaphthalene and other monomers in an organic solvent, adding a radical polymerization initiator thereto, and heating the solution for polymerization. In an alternative method, acetoxystyrene or acetoxyvinylnaphthalene is used instead, and after polymerization, the acetoxy group is deprotected by alkaline hydrolysis, for thereby converting the polymer product to polyhydroxystyrene or polyhydroxyvinylnaphthalene.

For alkaline hydrolysis, a base such as aqueous ammonia or triethylamine may be used. The reaction temperature is preferably −20 to 100° C., and more preferably 0 to 60° C. The reaction time is preferably 0.2 to 100 hours, and more preferably 0.5 to 20 hours.

The amounts of monomers in the monomer solution may be determined appropriate so as to provide the preferred fractions of repeat units.

It is now described how to use the polymer obtained by the above preparation method. The reaction solution resulting from polymerization reaction may be used as the final product. Alternatively, the polymer may be recovered in powder form through a purifying step such as re-precipitation step of adding the polymerization solution to a poor solvent and letting the polymer precipitate as powder, after which the polymer powder is used as the final product. It is preferred from the standpoints of operation efficiency and consistent quality to handle a polymer solution which is obtained by dissolving the powder polymer resulting from the purifying step in a solvent, as the final product.

The solvents which can be used herein are described in paragraphs [0144]-[0145] of JP-A 2008-111103. Specific examples thereof include ketones such as cyclohexanone and methyl-2-n-pentyl ketone; alcohols such as 3-methoxybutanol, 3-methyl-3-methoxybutanol, 1-methoxy-2-propanol, and 1-ethoxy-2-propanol; ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as γ-butyrolactone (GBL); keto-alcohols such as diacetone alcohol (DAA); high-boiling alcohols such as diethylene glycol, propylene glycol, glycerol, 1,4-butanediol, and 1,3-butanediol; and mixed solvents thereof.

A concentration of the polymer in the polymer solution is preferably 0.01 to 30 wt %, and more preferably 0.1 to 20 wt %.

Prior to use, the reaction solution or polymer solution is preferably filtered through a filter. Filtration is effective for consistent quality because foreign matter and gel which can cause defects are removed.

Suitable materials of which the filter is made include fluorocarbon, cellulose, nylon, polyester, and hydrocarbon base materials. Preferred for the filtration of a resist composition are filters made of fluorocarbons commonly known as Teflon®, hydrocarbons such as polyethylene and polypropylene, and nylon. While the pore size of the filter may be selected appropriate to comply with the desired cleanness, the filter preferably has a pore size of 100 nm or less, and more preferably 20 nm or less. A single filter may be used or a plurality of filters may be used in combination. Although the filtering method may be single pass of the solution, preferably the filtering step is repeated by flowing the solution in a circulating manner. In the polymer preparation process, the filtering step may be carried out any times, in any order and in any stage. The reaction solution as polymerized or the polymer solution may be filtered, preferably both are filtered.

The base polymer (B) may be used alone or as a blend of two or more polymers which differ in compositional ratio, Mw, and/or Mw/Mn. The base polymer (B) may contain a hydrogenated product of a ring-opening metathesis polymer in addition to the polymer described above. For the ring-opening metathesis polymer reference is made to JP-A 2003-66612.

### (C) Organic Solvent

The chemically amplified resist composition of the present invention may comprise an organic solvent as a component (C). The organic solvent (C) is not particularly limited as long as the foregoing and other components are soluble therein. Specific examples of the organic solvent include ketones such as cyclopentanone, cyclohexanone, and methyl-2-n-pentyl ketone; alcohols such as 3-methoxybutanol, 3-methyl-3-methoxybutanol, 1-methoxy-2-propanol, and 1-ethoxy-2-propanol; keto-alcohols such as DAA; ethers such as PGME, ethylene glycol monomethyl ether, propylene glycol monoethyl ether, ethylene glycol monoethyl ether, propylene glycol dimethyl ether, and diethylene glycol dimethyl ether; esters such as PGMEA, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; lactones such as GBL; and mixed solvents thereof.

Among these organic solvents, 1-ethoxy-2-propanol, PGMEA, cyclohexanone, GBL, DAA, and mixed solvents thereof are preferred because the base polymer (B) is most soluble therein.

In the chemically amplified resist composition of the present invention, the content of the organic solvent (C) is preferably 200 to 5,000 parts by weight, and more preferably 400 to 3,500 parts by weight per 80 parts by weight of the base polymer (B). The organic solvent (C) may be used alone or in admixture.

### (D) Photoacid Generator

The chemically amplified resist composition of the present invention may comprise a photoacid generator as a component (D). The photoacid generator is not particularly limited as long as it is a compound capable of generating an acid having an acid strength higher than that of a sulfonic acid in which a quencher of the component (A) is generated by high-energy radiation, and a photoacid generator capable of generating an acid having a pKa of the generated acid of pKa≤−2.0 is preferred.

The preferred photoacid generator is a salt having the formula (2) or (3).

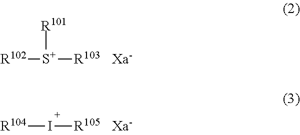

In formula (2), R¹⁰¹ to R¹⁰⁵ are each independently a C₁-C₂₀ hydrocarbyl group which may contain a heteroatom. Any two of R¹⁰¹, R¹⁰², and R¹⁰³ may bond together to form a ring with the sulfur atoms to which they are attached. Specific examples of the hydrocarbyl group are as exemplified above for the hydrocarbyl groups R^{ct1} to R^{ct5} in the descriptions of formulae (Z-1) and (Z-2).

Specific examples of the cation of the sulfonium salt having formula (2) are as exemplified above for the sulfonium cation having formula (Z-1). Specific examples of the cation of the iodonium salt having formula (3) are as exemplified above for the iodonium cation having formula (Z-2).

In formulae (2) and (3), Xa⁻ is an anion of a strong acid. Specific examples of the anion of the strong acid include those having any of formulae (c1-1) to (c1-5).

The photoacid generator as the component (D) is also preferably a photoacid generator having the formula (4).

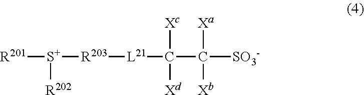

In formula (4), R²⁰¹ and R²⁰² are each independently a C₁-C₃₀ hydrocarbyl group which may contain a heteroatom. R²⁰³ is a C₁-C₃₀ hydrocarbylene group which may contain a heteroatom. Any two of R²⁰¹, R²⁰², and R²⁰³ may bond together to form a ring with the sulfur atoms to which they are attached.

The C₁-C₃₀ hydrocarbyl groups R²⁰¹ and R²⁰² may be saturated or unsaturated and straight, branched, or cyclic. Specific examples thereof include C₁-C₃₀ alkyl groups such as a methyl group, an ethyl group, an n-propyl group, an isopropyl group, an n-butyl group, an isobutyl group, a sec-butyl group, a tert-butyl group, a tert-pentyl group, an n-pentyl group, an n-hexyl group, an n-octyl group, a 2-ethylhexyl group, an n-nonyl group, and an n-decyl group; C₃-C₃₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, an oxanorbornyl group, a tricyclo[5.2.1.0^{2,6}]decyl group, and an adamantyl group; C₆-C₃₀ aryl groups such as a phenyl group, a methylphenyl group, an ethylphenyl group, an n-propylphenyl group, an isopropylphenyl group, an n-butylphenyl group, an isobutylphenyl group, a sec-butylphenyl group, a tert-butylphenyl group, a naphthyl group, a methylnaphthyl group, an ethylnaphthyl group, an n-propylnaphthyl group, an isopropylnaphthyl group, an n-butylnaphthyl group, an isobutylnaphthyl group, a sec-butylnaphthyl group, a tert-butylnaphthyl group, and an anthracenyl group; and combinations thereof. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

The C₁-C₃₀ hydrocarbylene group R²⁰³ may be saturated or unsaturated and straight, branched, or cyclic. Specific examples thereof include C₁-C₃₀ alkanediyl groups such as a methanediyl group, an ethane-1,1-diyl group, an ethane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, a dodecane-1,12-diyl group, a tridecane-1,13-diyl group, a tetradecane-1,14-diyl group, a pentadecane-1,15-diyl group, a hexadecane-1,16-diyl group, and a heptadecane-1,17-diyl group; C₃-C₃₀ cyclic saturated hydrocarbylene groups such as a cyclopentanediyl group, a cyclohexanediyl group, a norbornanediyl group, and an adamantanediyl group; and arylene groups such as a phenylene group, a methylphenylene group, an ethylphenylene group, an n-propylphenylene group, an isopropylphenylene group, an n-butylphenylene group, an isobutylphenylene group, a sec-butylphenylene group, a tert-butylphenylene group, a naphthylene group, a methylnaphthylene group, an ethylnaphthylene group, an n-propylnaphthylene group, an isopropylnaphthylene group, an n-butylnaphthylene group, an isobutylnaphthylene group, a sec-butylnaphthylene group, and a tert-butylnaphthylene group. In the hydrocarbylene group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a cyano group, a fluorine atom, a chlorine atom, a bromine atom, an iodine atom, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group. The heteroatom is preferably an oxygen atom.

In formula (4), L²¹ is a single bond, an ether bond, or a C₁-C₂₀ hydrocarbylene group which may contain a heteroatom. The hydrocarbylene group may be saturated or unsaturated and straight, branched, or cyclic. Specific examples thereof are as exemplified above for the hydrocarbylene group R²⁰³.

In formula (4), X^{a}, X^{b}, X^{c}, and X^{d} are each independently a hydrogen atom, a fluorine atom, or a trifluoromethyl group. Provided that at least one of X^{a}, X^{b}, X^{c}, and X^{d} is a fluorine atom or a trifluoromethyl group.

The photoacid generator having formula (4) is preferably a photoacid generator having the formula (4′).

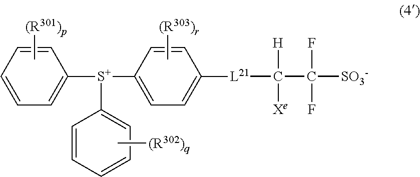

In formula (4′), L²¹ is as defined above. Xe is a hydrogen atom or a trifluoromethyl group, and preferably a trifluoromethyl group. R³⁰¹, R³⁰², and R³⁰³ are each independently a hydrogen atom or a C₁-C₂₀ hydrocarbyl group which may contain a heteroatom. The hydrocarbyl group may be saturated or unsaturated and straight, branched, or cyclic. Specific examples thereof are as exemplified for the hydrocarbyl group R^{fa1} in formula (c1-1-1). p and q are each independently 0, 1, 2, 3, 4, or 5, and r is 0, 1, 2, 3, or 4.

Specific examples of the photoacid generator having formula (4) include those exemplified for the photoacid generator having formula (2) in JP-A 2017-26980.

Among the photoacid generators, a photoacid generator containing an anion having formula (c1-1-1) or (c1-4) has small acid diffusion and excellent solubility in a solvent, and is particularly preferred. In addition, a photoacid generator having formula (4′) has extremely small acid diffusion, and is particularly preferred.

When the chemically amplified resist composition of the present invention contains the photoacid generator (D), the content thereof is preferably 0.1 to 40 parts by weight, and more preferably 0.5 to 20 parts by weight per 80 parts by weight of the base polymer (B). When the amount of the photoacid generator (D) added is in the above range, the resolution is excellent, and there is no possibility that a problem of foreign matter occurs after development or during peeling of the resist film, which is preferable. The photoacid generator (D) may be used alone or in admixture.

### (E) Other Quencher

The chemically amplified resist composition of the present invention may contain a quencher other than the component (A) as a component (E) (hereinafter, also referred to as other quencher).

Specific examples of the other quencher as the component (E) include onium salts having the formula (5) or (6).

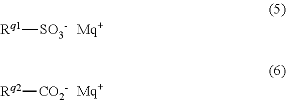

In formula (5), R^{q1} is a hydrogen atom or a C₁-C₄₀ hydrocarbyl group which may contain a heteroatom, exclusive of the group wherein a hydrogen atom bonded to the carbon atom at α-position relative to the sulfo group is substituted by a fluorine atom or a fluoroalkyl group. In formula (6), R^{q2} is a hydrogen atom or a C₁-C₄₀ hydrocarbyl group which may contain a heteroatom.

Examples of the C₁-C₄₀ hydrocarbyl group R⁴¹ include C₁-C₄₀ alkyl groups such as a methyl group, an ethyl group, an n-propyl group, an isopropyl group, an n-butyl group, an isobutyl group, a sec-butyl group, a tert-butyl group, an n-pentyl group, a tert-pentyl group, an n-hexyl group, an n-octyl group, a 2-ethylhexyl group, an n-nonyl group, and an n-decyl group; C₃-C₄₀ cyclic saturated hydrocarbyl groups such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decyl group, and an adamantyl group; and C₆-C₄₀ aryl groups such as a phenyl group, a naphthyl group, and an anthracenyl group. In the hydrocarbyl group, some or all hydrogen atoms may be substituted by a group containing a heteroatom such as an oxygen atom, a sulfur atom, a nitrogen atom, or a halogen atom, and some —CH₂— may be replaced by a group containing a heteroatom such as an oxygen atom, a sulfur atom, or a nitrogen atom so that the group may contain a hydroxy group, a fluorine atom, a chlorine atom, a bromine atom, an iodine atom, a cyano group, a carbonyl group, an ether bond, an ester bond, a sulfonate ester bond, a carbonate bond, a lactone ring, a sultone ring, a carboxylic anhydride (—C(═O)—O—C(═O)—), or a haloalkyl group.

Specific examples of the hydrocarbyl group R^{q2} include, in addition to the substituents exemplified as specific examples of R^{q1}, fluorinated saturated hydrocarbyl groups such as a trifluoromethyl group and a trifluoroethyl group, and fluorinated aryl groups such as a pentafluorophenyl group and a 4-trifluoromethylphenyl group.

Specific examples of the anion of the onium salt having formula (5) are shown below. but are not limited thereto.

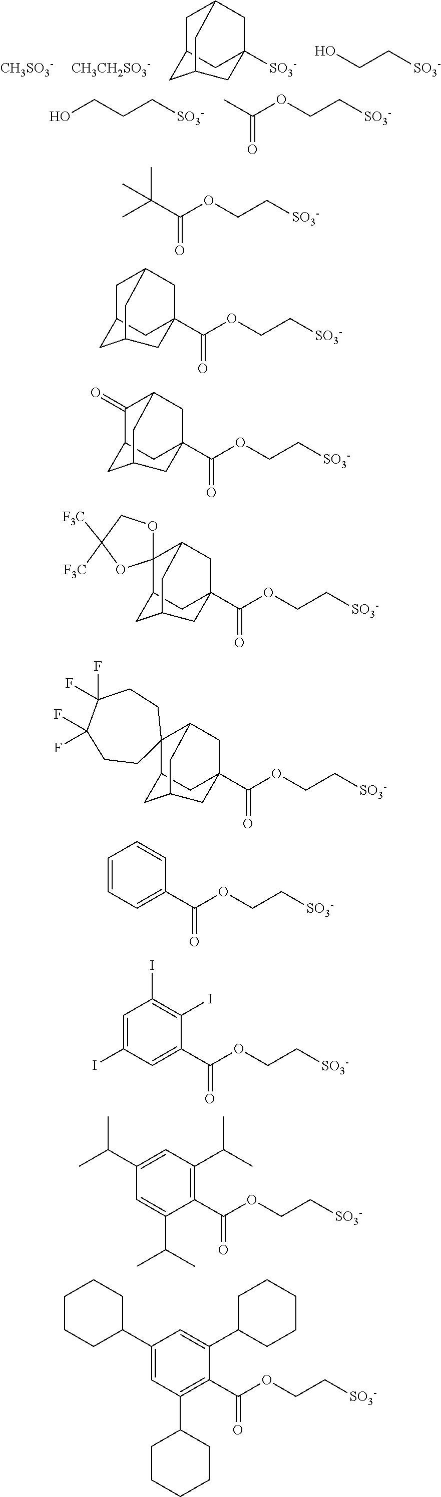

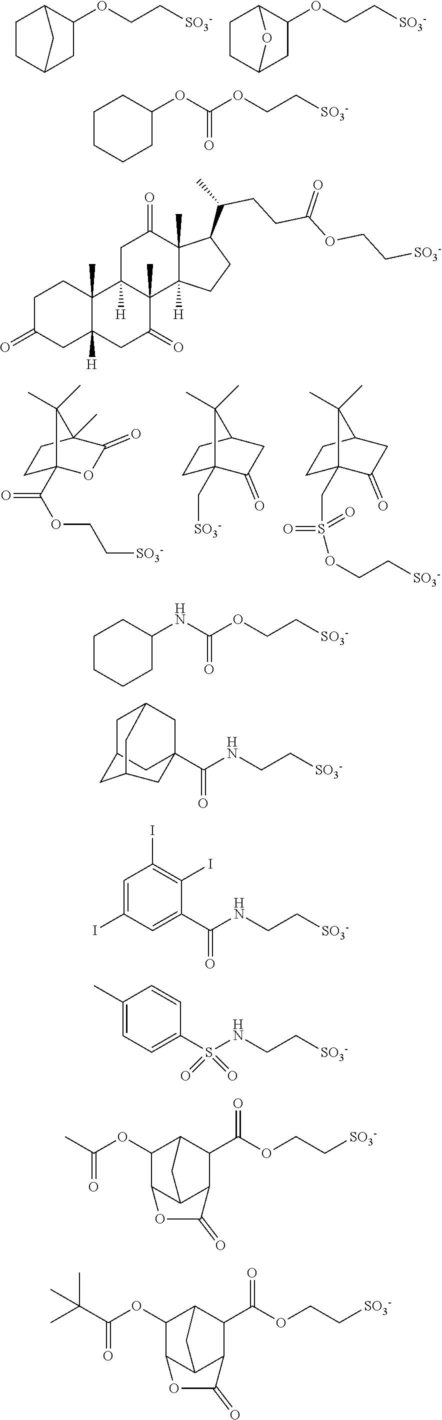

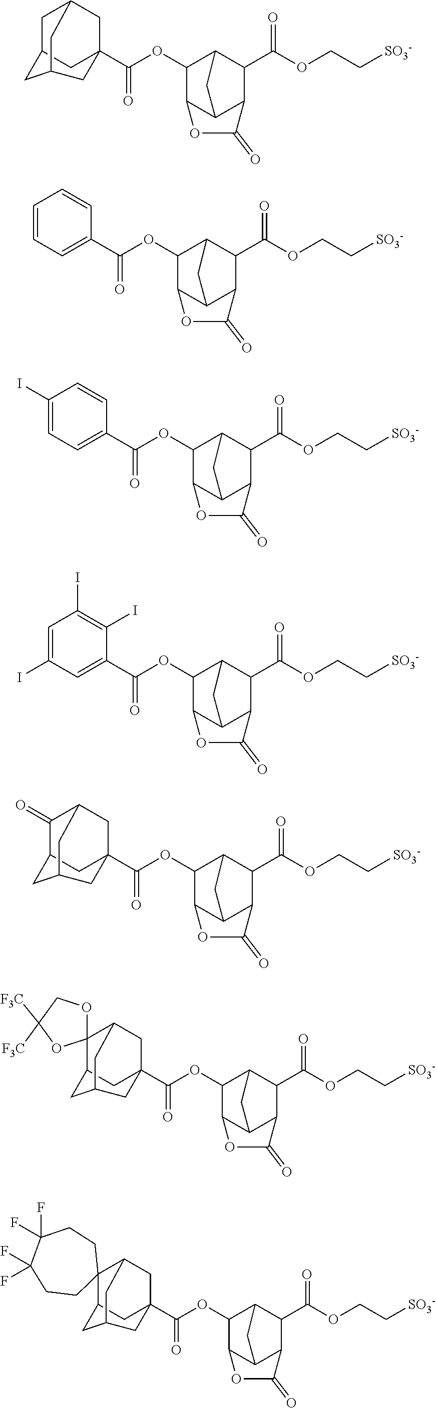

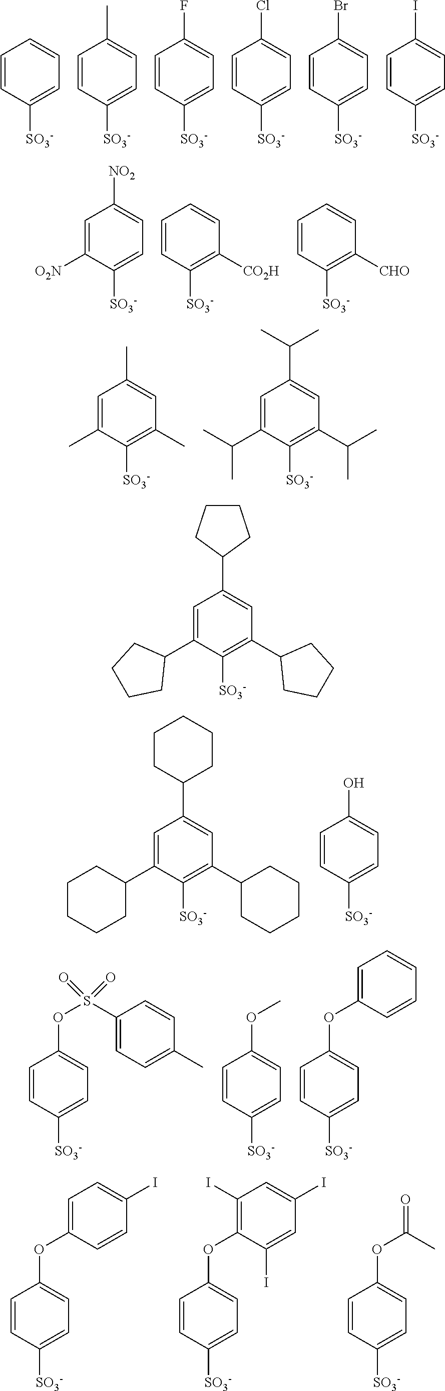

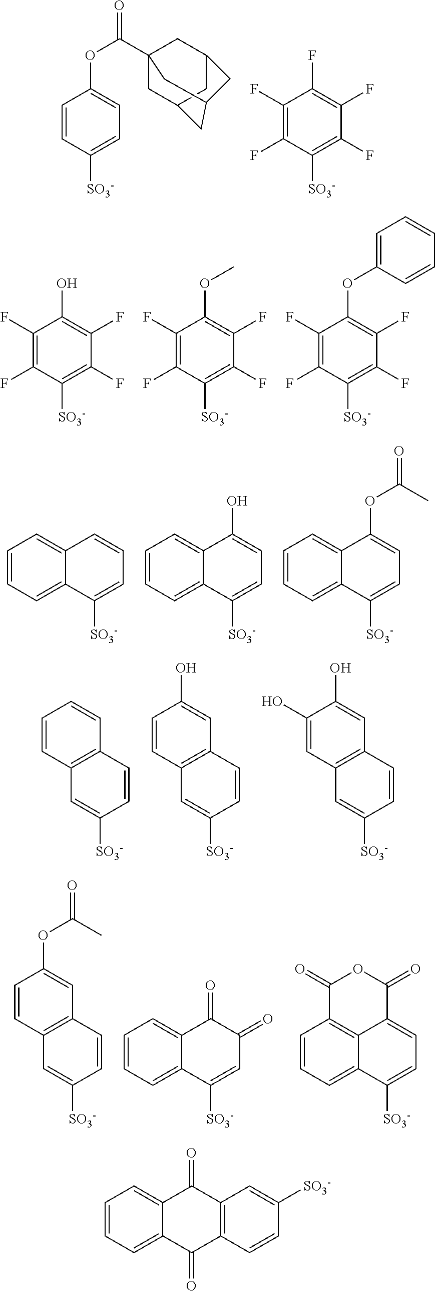

Specific examples of the anion of the onium salt having formula (6) are shown below. but are not limited thereto.

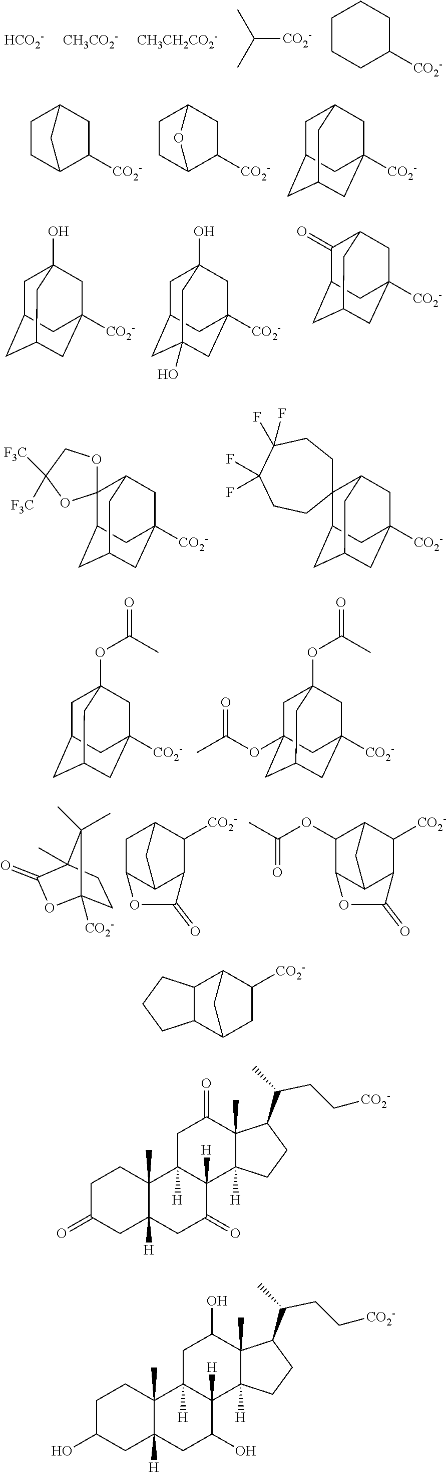

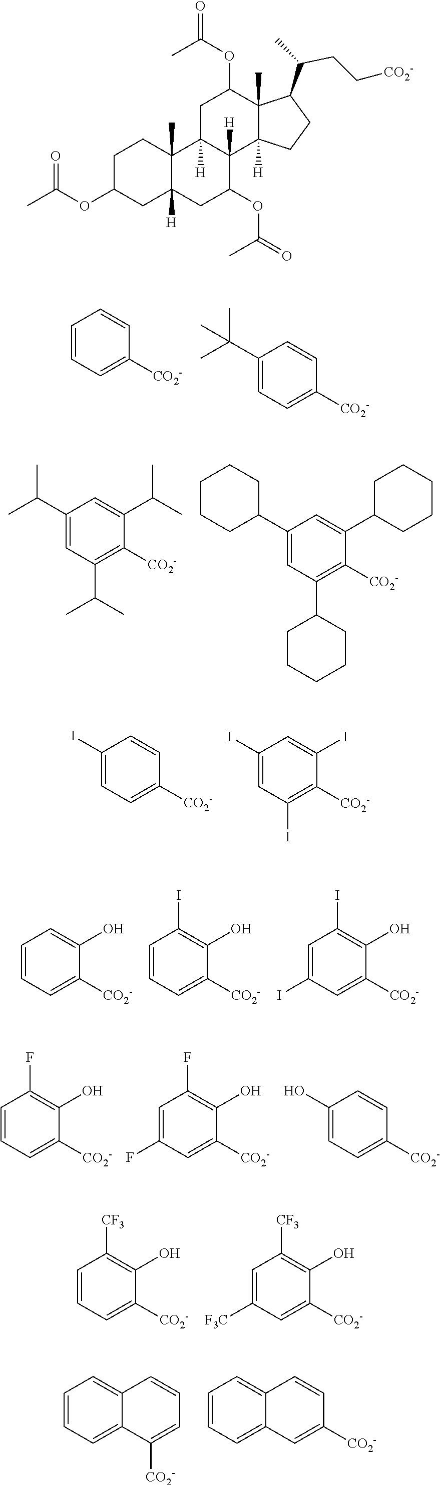

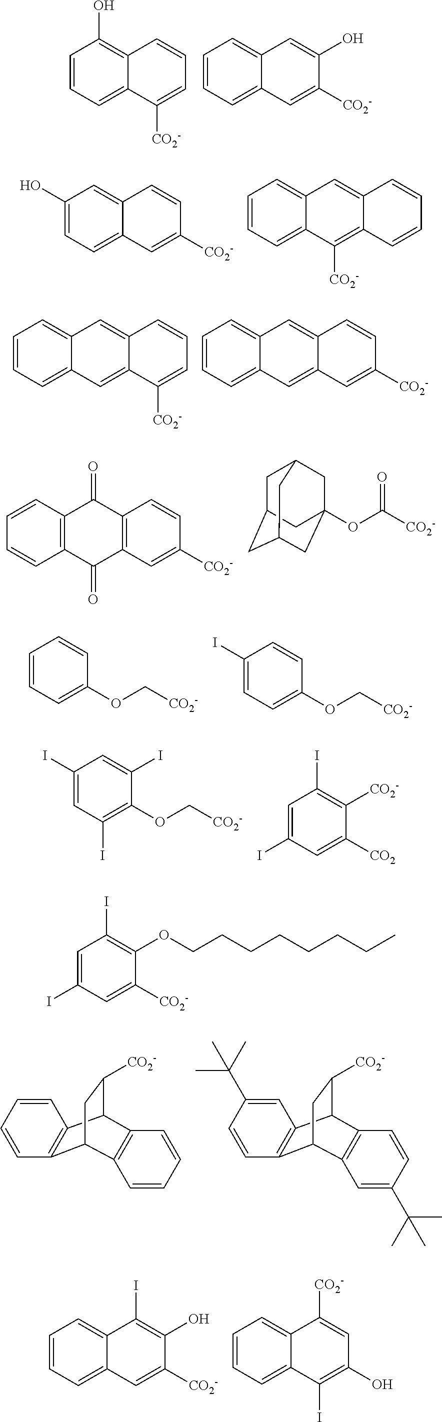

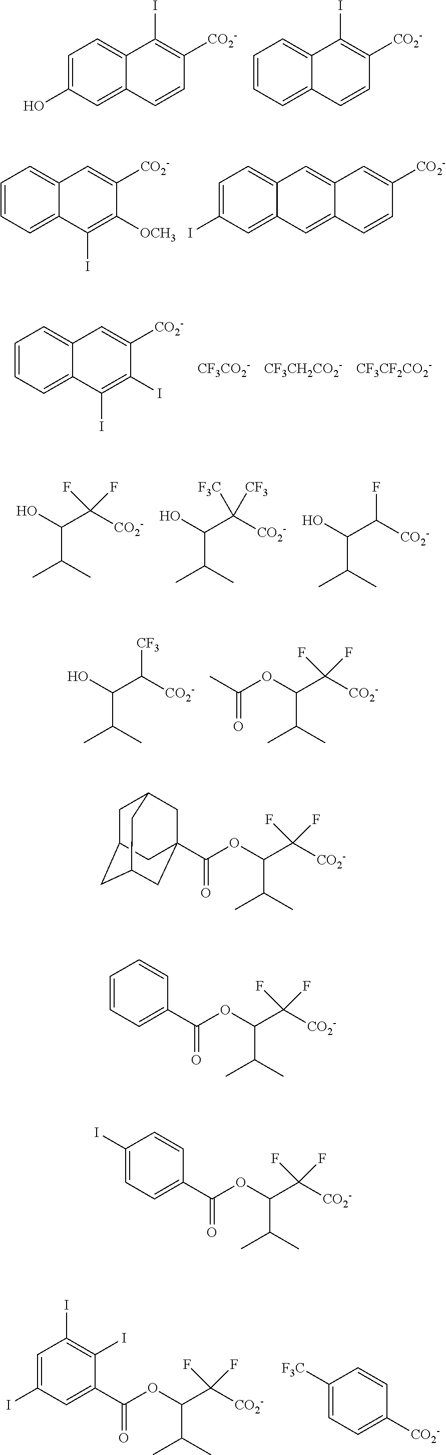

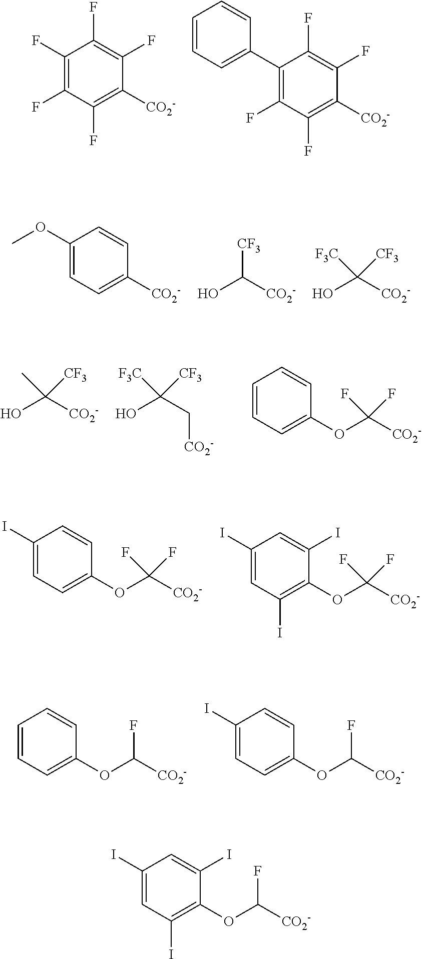

In formulae (5) and (6), Mq⁺ is an onium cation. The onium cation is preferably a sulfonium cation having the formula (Z-1), an iodonium cation having the formula (Z-2), or an ammonium cation having the formula (Z-3).

Specific examples of the onium salt having formula (5) or (6) include any combination of the anion and the cation described above. These onium salts are easily prepared by an ion exchange reaction using a known organic chemical method. For the ion exchange reaction, for example, JP-A 2007-145797 can be referred to.

The onium salt having formula (5) or (6) acts as a quencher in the chemically amplified resist composition of the present invention. This is because each counter anion of the onium salt is a conjugated base of a weak acid. As used herein, the weak acid indicates an acidity insufficient to deprotect an acid labile group from an acid labile group-containing unit in the base polymer. The onium salt having formula (5) or (6) functions as a quencher when used in combination with an onium salt type photoacid generator having a conjugated base of a strong acid (typically sulfonic acid) as the counter anion. In a system using a mixture of an onium salt capable of generating a strong acid (typically sulfonic acid) and an onium salt capable of generating a weak acid (typically carboxylic acid), if the strong acid generated from the photoacid generator upon exposure to high-energy radiation collides with the unreacted onium salt having a weak acid anion, then a salt exchange occurs whereby the weak acid is released and an onium salt having a strong acid anion is formed. In this course, the strong acid is exchanged into the weak acid having a low catalysis, incurring apparent deactivation of the acid for enabling to control acid diffusion.

As the other quencher as the component (E), onium salts having sulfonium cation and phenoxide anion sites in a common molecule as described in JP 6848776, onium salts having sulfonium cation and carboxylate anion sites in a common molecule as described in JP 6583136 and JP-A 2020-200311, and onium salts having iodonium cation and carboxylate anion sites in a common molecule as described in JP 6274755 can be used.

If a photoacid generator capable of generating a strong acid is an onium salt, an exchange from the strong acid generated upon exposure to high-energy radiation to a weak acid can take place as mentioned above, but it rarely happens that the weak acid generated upon exposure to high-energy radiation collides with the unreacted onium salt capable of generating a strong acid to induce a salt exchange. This is because of the phenomenon that an onium cation is more likely to form an ion pair with a stronger acid anion.

When the onium salt having formula (5) or (6) is used as the other quencher of the component (E) in the chemically amplified resist composition of the present invention, the content of the onium salt is preferably 0.1 to 20 parts by weight, and more preferably 0.1 to 10 parts by weight per 80 parts by weight of the base polymer (B). As long as the content of the onium salt type quencher is in the range, a satisfactory resolution is available without a substantial lowering of sensitivity. The onium salt having formula (5) or (6) may be used alone or in admixture.

The chemically amplified resist composition of the present invention may comprise a nitrogen-containing compound as the other quencher of the component (E). Specific examples of the nitrogen-containing compound include primary, secondary, and tertiary amine compounds, specifically amine compounds having a hydroxy group, an ether bond, an ester bond, a lactone ring, a cyano group, or a sulfonate ester bond, as described in paragraphs [0146]-[0164] of JP-A 2008-111103. In addition, as in the compound described in JP 3790649, a compound in which a primary or secondary amine is protected by a carbamate group can also be used.

A sulfonic acid sulfonium salt having a nitrogen-containing substituent may also be used as the nitrogen-containing compound. This compound functions as a quencher in the unexposed region, but as a so-called photo-degradable base in the exposed region because it loses the quencher function in the exposed region due to neutralization thereof with the acid generated by itself. Using a photo-degradable base, the contrast between exposed and unexposed regions can be further enhanced. With respect to the photo-degradable base, reference may be made to JP-A 2009-109595 and JP-A 2012-46501, for example.

When the nitrogen-containing compound is used as the other quencher of the component (E) in the chemically amplified resist composition of the present invention, the content of the nitrogen-containing compound is preferably 0.001 to 12 parts by weight, and more preferably 0.01 to 8 parts by weight per 80 parts by weight of the base polymer (B). The nitrogen-containing compound may be used alone or in admixture.

### (F) Surfactant

The chemically amplified resist composition of the present invention may further comprise a surfactant as a component (F). The surfactant as the component (F) is preferably a surfactant which is insoluble or substantially insoluble in water and soluble in an alkaline developer, and a surfactant which is insoluble or substantially insoluble in water and an alkaline developer. For the surfactant, reference should be made to those compounds described in JP-A 2010-215608 and JP-A 2011-16746.

As the surfactant which is insoluble or substantially insoluble in water and an alkaline developer, among the surfactants described in the patent documents, FC-4430 (3M), Surflon® S-381 (AGC Seimi Chemical Co., Ltd.), Olfine® E1004 (Nissin Chemical Co., Ltd.), KH-20 and KH-30 (AGC Seimi Chemical Co., Ltd.), and an oxetane ring-opening polymer having the formula (surf-1) are preferred.

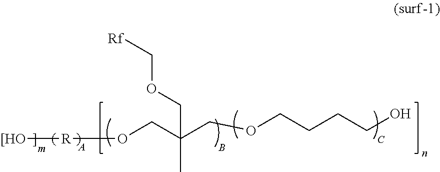

It is provided herein that R, Rf, A, B, C, m, and n are applied to only formula (surf-1), independent of their descriptions other than for the surfactant. R is a di- to tetra-valent C₂-C₅ aliphatic group. Examples of the divalent aliphatic group include an ethylene group, a 1,4-butylene group, a 1,2-propylene group, a 2,2-dimethyl-1,3-propylene group, and a 1,5-pentylene group. Examples of the tri- and tetra-valent group are shown below.

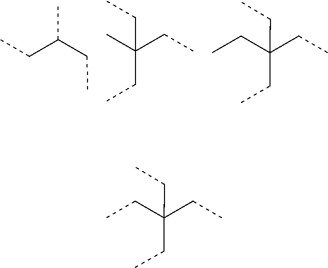

Herein the broken line denotes a point of attachment. These formulae are partial structures derived from glycerol, trimethylol ethane, trimethylol propane, and pentaerythritol, respectively.

Among them, a 1,4-butylene group and a 2,2-dimethyl-1,3-propylene group are preferred.

Rf is a trifluoromethyl group or a pentafluoroethyl group, and preferably a trifluoromethyl group. m is an integer of 0 to 3, n is an integer of 1 to 4, and the sum of m and n, which represents the valence of R, is an integer of 2 to 4. A is 1. B is an integer of 2 to 25, and preferably an integer of 4 to 20. C is an integer of 0 to 10, and preferably 0 or 1. The formula (surf-1) does not prescribe the arrangement of respective constituent units while they may be arranged either blockwise or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist film surface for achieving a function of minimizing water penetration or leaching. The surfactant is also effective for preventing water-soluble components from being leached out of the resist film for minimizing any damage to the exposure tool. The surfactant becomes solubilized during aqueous alkaline development following exposure and PEB, and thus forms few or no foreign matter which becomes defects. The preferred surfactant is a polymeric surfactant which is insoluble or substantially insoluble in water, but soluble in alkaline developer, also referred to as “hydrophobic resin” in this sense, and especially which is water repellent and enhances water sliding.

Specific examples of the polymeric surfactant include those containing repeat units of at least one type selected from the formulae (7A) to (7E).

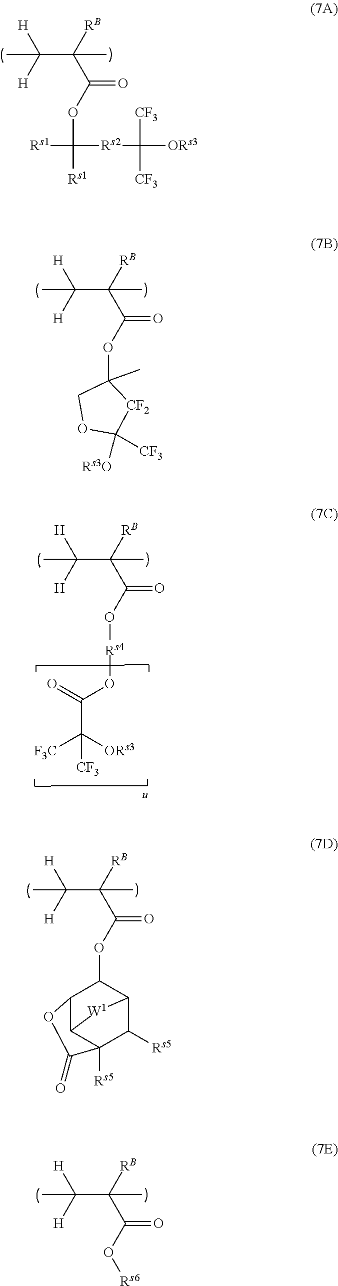

In formulae (7A) to (7E), RB is a hydrogen atom, a fluorine atom, a methyl group, or a trifluoromethyl group. W¹ is-CH₂—, —CH₂CH₂—, —O—, or two separate —H. R^{s1} is each independently a hydrogen atom or a C₁-C₁₀ hydrocarbyl group. R^{s2} is a single bond or a straight or branched C₁-C₈ hydrocarbylene group. R^{s3} is each independently a hydrogen atom, a C₁-C₁₅ hydrocarbyl group, a fluorinated hydrocarbyl group, or an acid labile group. When R^{s3} is a hydrocarbyl group or fluorinated hydrocarbyl group, an ether bond or a carbonyl group may intervene in a carbon-carbon bond. R^{s4} is a C₁-C₂₀ (u+1)-valent hydrocarbon or fluorinated hydrocarbon group. u is 1, 2, or 3. R^{s5} is each independently a hydrogen atom or a group: —C(═O)—O—R^{sa}. R^{sa} is a C₁-C₂₀ fluorinated hydrocarbyl group. When R^{s6} is a C₁-Cis hydrocarbyl group or fluorinated hydrocarbyl group, an ether bond or a carbonyl group may intervene in a carbon-carbon bond.

The C₁-C₁₀ hydrocarbyl group R^{s1} is preferably a saturated hydrocarbyl group, and may be straight, branched, or cyclic. Specific examples thereof include C₁-C₁₀ alkyl groups such as a methyl group, an ethyl group, an n-propyl group, an isopropyl group, an n-butyl group, an isobutyl group, a sec-butyl group, a tert-butyl group, an n-pentyl group, an n-hexyl group, an n-heptyl group, an n-octyl group, an n-nonyl group, and an n-decyl group; and C₃-C₁₀ cyclic saturated hydrocarbyl groups such as a cyclopropyl group, a cyclobutyl group, a cyclopentyl group, a cyclohexyl group, an adamantyl group, and a norbornyl group. Among them, C₁-C₆ hydrocarbyl groups are preferred.

The hydrocarbylene group R^{s2} is preferably a saturated hydrocarbylene group, and may be straight, branched, or cyclic. Specific examples thereof include a methylene group, an ethylene group, a propylene group, a butylene group, and a pentylene group.

The hydrocarbyl group R^{s3} or R^{s6} may be saturated or unsaturated and straight, branched, or cyclic. Specific examples thereof include aliphatic unsaturated hydrocarbyl groups such as a saturated hydrocarbyl group, an alkenyl group, and an alkynyl group, and a saturated hydrocarbyl group is preferred. Specific examples of the saturated hydrocarbyl group include undecyl group, a dodecyl group, a tridecyl group, a tetradecyl group, and a pentadecyl group, as well as those exemplified for the hydrocarbyl group represented by R^{s1}. Examples of the fluorinated hydrocarbyl group R^{s3} or R^{s6} include groups in which some or all of hydrogen atoms bonded to carbon atoms of the hydrocarbyl group described above are substituted by fluorine atoms. As described above, an ether bond or a carbonyl group may intervene in a carbon-carbon bond.

Specific examples of the acid labile group represented by R^{s3} include groups of the above formulae (AL-3) to (AL-5), a trialkylsilyl group in which each alkyl group is a C₁-C₆ alkyl group, and a C₄-C₂₀ oxo group-containing alkyl group.

The (u+1)-valent hydrocarbon or fluorinated hydrocarbon group represented by R^{s4} may be straight, branched, or cyclic and, specific examples thereof include the foregoing hydrocarbyl group or fluorinated hydrocarbyl group from which “u” number of hydrogen atoms are eliminated.

The fluorinated hydrocarbyl group represented by R^{sa} may be straight, branched, or cyclic, and is preferably saturated. Specific examples thereof include the foregoing hydrocarbyl groups in which some or all hydrogen atoms are substituted by fluorine atoms. Specific examples thereof include a trifluoromethyl group, a 2,2,2-trifluoroethyl group, a 3,3,3-trifluoro-1-propyl group, a 3,3,3-trifluoro-2-propyl group, a 2,2,3,3-tetrafluoropropyl group, a 1,1,1,3,3,3-hexafluoroisopropyl group, a 2,2,3,3,4,4,4-heptafluorobutyl group, a 2,2,3,3,4,4,5,5-octafluoropentyl group, a 2,2,3,3,4,4,5,5,6,6,7,7-dodecafluoroheptyl group, a 2-(perfluorobutyl)ethyl group, a 2-(perfluorohexyl)ethyl group, a 2-(perfluorooctyl)ethyl group, and a 2-(perfluorodecyl)ethyl group.

Specific examples of the repeat units having any one of formulae (7A) to (7E) are shown below, but are not limited thereto. Herein R^{B} is as defined above.

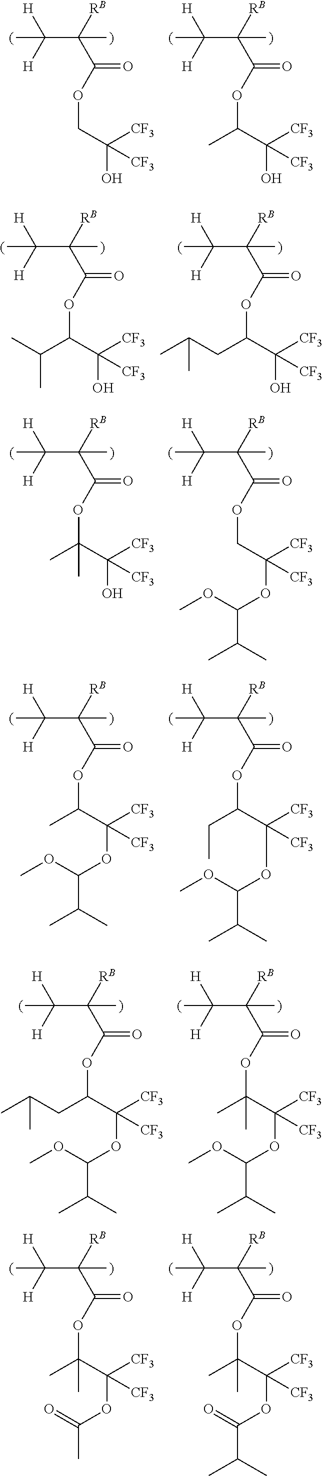

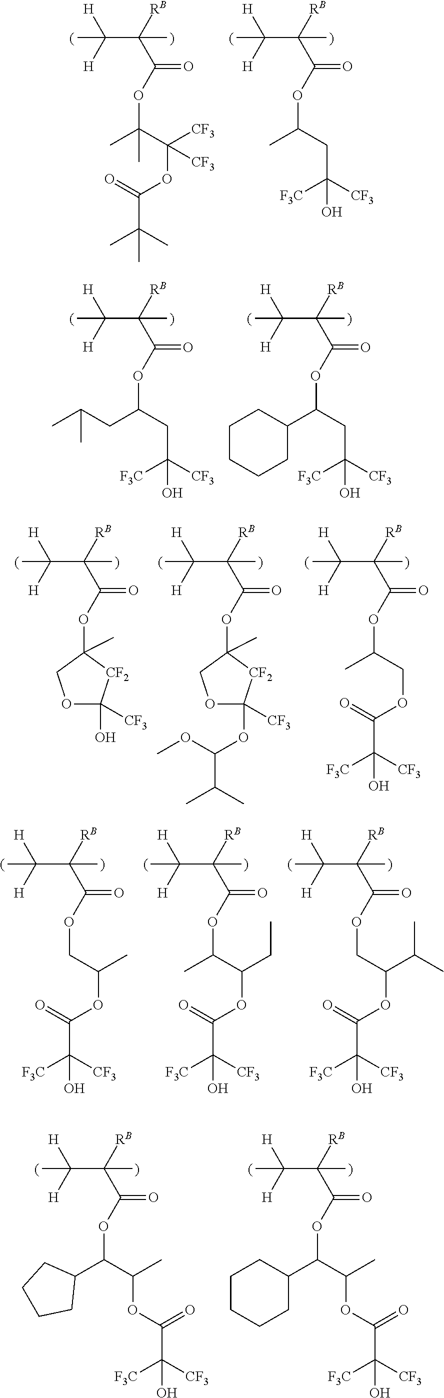

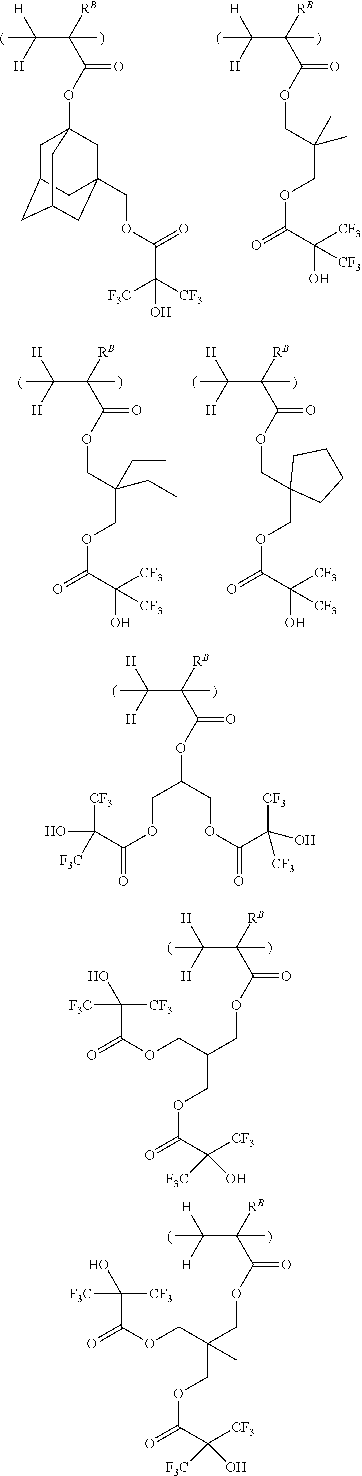

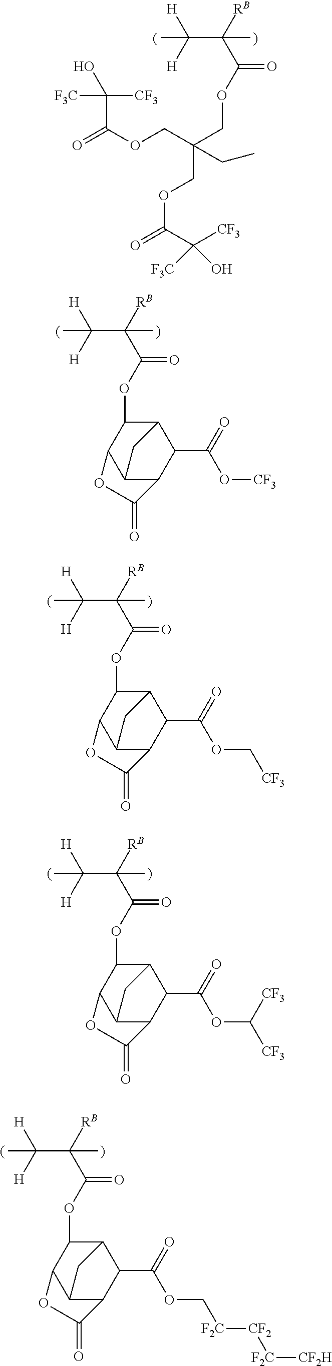

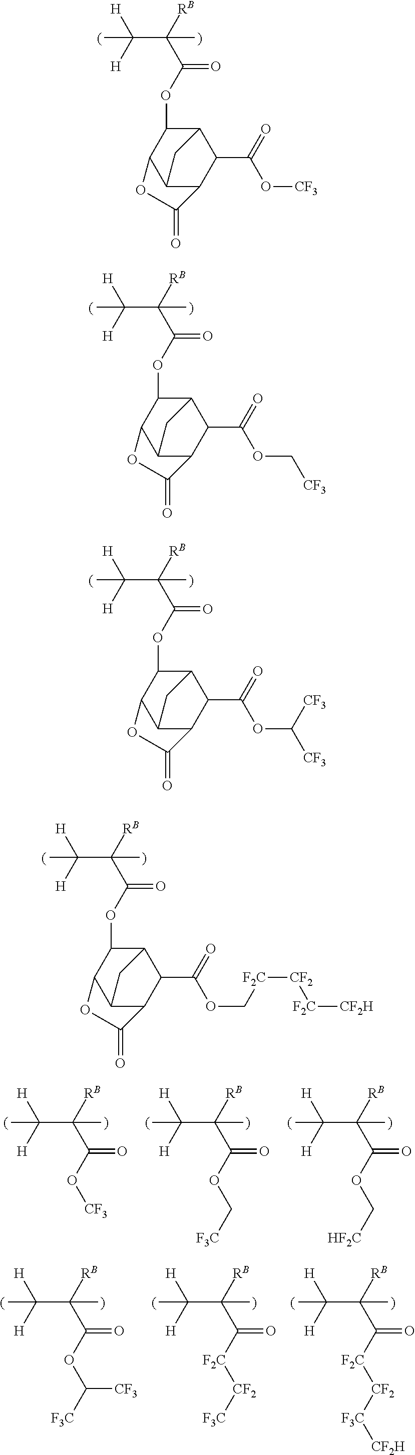

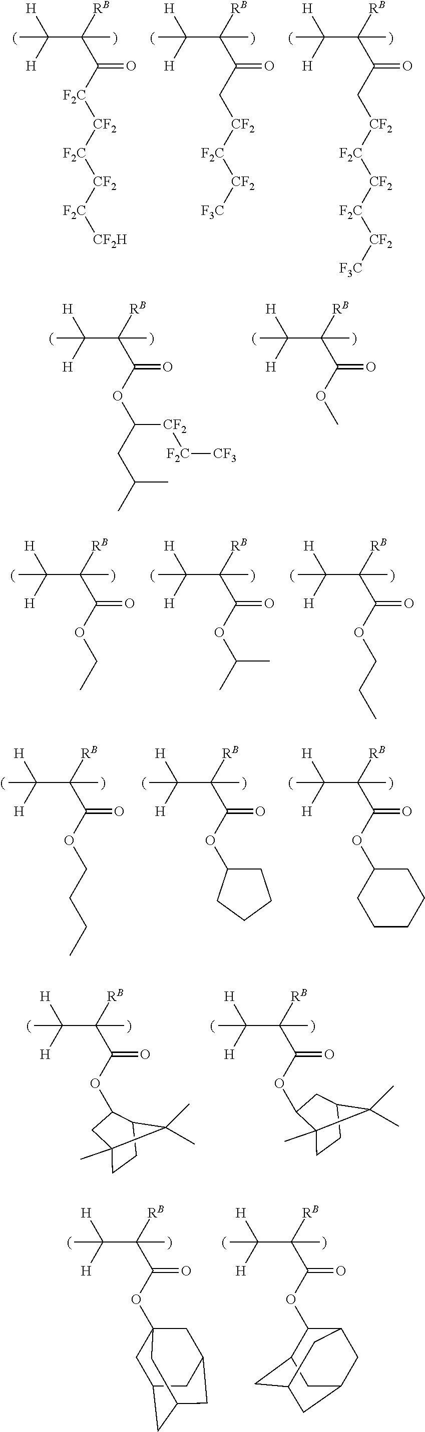

The polymeric surfactant may further contain repeat units other than the repeat units having formulae (7A) to (7E). Specific examples of the other repeat units include those derived from methacrylic acid and α-trifluoromethylacrylic acid derivatives. In the polymeric surfactant, the content of the repeat units having formulae (7A) to (7E) is preferably 20 mol % or more, more preferably 60 mol % or more, and still more preferably 100 mol % of the overall repeat units.

A Mw of the polymeric surfactant is preferably 1,000 to 500,000, and more preferably 3,000 to 100,000. Mw/Mn is preferably 1.0 to 2.0, and more preferably 1.0 to 1.6.

Examples of the method for synthesizing the polymeric surfactant include a method in which a monomer containing repeat units having formulae (7A) to (7E) and, if necessary, an unsaturated bond providing other repeat units is dissolved in an organic solvent, a radical initiator is added, and heating is performed for polymerization. Specific examples of the organic solvent used in the polymerization include toluene, benzene, THF, diethyl ether, and dioxane. Specific examples of the polymerization initiator include AIBN, 2,2′-azobis(2,4-dimethylvaleronitrile), dimethyl-2,2-azobis(2-methylpropionate), benzoyl peroxide, and lauroyl peroxide. The reaction temperature is preferably 50 to 100° C. The reaction time is preferably 4 to 24 hours. The acid labile group that has been incorporated in the monomer may be kept as such, or polymerization may be followed by protection or partial protection.

During the synthesis of the polymeric surfactant, any known chain transfer agent such as dodecyl mercaptan or 2-mercaptoethanol may be used for molecular weight control purpose. The amount of chain transfer agent added is preferably 0.01 to 10 mol % based on the total moles of monomers to be polymerized.

When the chemically amplified resist composition of the present invention contains the surfactant (F), the content thereof is preferably 0.1 to 50 parts by weight, and more preferably 0.5 to 10 parts by weight per 80 parts by weight of the base polymer (B). If the content of the surfactant (F) is 0.1 parts by weight or more, the receding contact angle between the surface of the resist film and water is sufficiently improved, and if the content of the surfactant is 50 parts by weight or less, a rate of dissolution of the surface of the resist film in the developer is low, and the height of a small-size pattern formed therein is sufficiently maintained. The surfactant (F) may be used alone or in admixture.

### (G) Other Components

The chemically amplified resist composition of the present invention may comprise, as (G) other components, a compound which is decomposed by an acid to generate an acid (acid amplifier compound), an organic acid derivative, a fluorinated alcohol, and a compound having a Mw of 3,000 or less which changes its solubility in developer by the action of an acid (dissolution inhibitor). As the acid amplifier compound, the compound described in JP-A 2009-269953 or JP-A 2010-215608 can be referred to. When the acid amplifier compound is contained, the content thereof is preferably 0 to 5 parts by weight, and more preferably 0 to 3 parts by weight per 80 parts by weight of the base polymer (B). When the content is too large, it is difficult to control acid diffusion, and resolution and pattern profile may be degraded. As the organic acid derivative, the fluorinated alcohol, and the dissolution inhibitor, the compound described in JP-A 2009-269953 or JP-A 2010-215608 can be referred to.

### Pattern Forming Process

A pattern forming process of the present invention comprises the steps of applying the chemically amplified resist composition described above onto a substrate to form a resist film thereon, exposing the resist film to high-energy radiation, and developing the exposed resist film in a developer.

The substrate used herein may be a substrate for integrated circuitry fabrication, for example, Si, SiO₂, SiN, SiON, TiN, WSi, BPSG, SOG, organic antireflective film, and the like, or a substrate for mask circuitry fabrication, for example, Cr, CrO, CrON, MoSi₂, SiO₂, and the like.

The resist film can be formed by, for example, applying the chemically amplified resist composition onto a substrate by a method such as spin coating so as to have a film thickness of preferably 0.05 to 2 μm, and performing prebaking on a hotplate at preferably 60 to 150° C. for 1 to 10 minutes, and more preferably 80 to 140° C. for 1 to 5 minutes.

Examples of the high-energy radiation used for exposure of the resist film include KrF excimer laser, ArF excimer laser, EB, and EUV of wavelength 3 to 15 nm. On use of KrF excimer laser, ArF excimer laser, or EUV, the resist film is exposed through a mask having a desired pattern, preferably in a dose of 1 to 200 mJ/cm², and more preferably 10 to 100 mJ/cm². On use of EB, a pattern may be written directly or through a mask having the desired pattern, preferably in a dose of 1 to 300 μC/cm², and more preferably 10 to 200 μC/cm².

The exposure can be performed by conventional lithography whereas the immersion lithography of holding a liquid having a refractive index of 1.0 or more between the resist film and the projection lens can be employed. In this case, a protective film which is insoluble in water may be formed on the resist film.

While the water-insoluble protective film serves to prevent any components from being leached out of the resist film and to improve water sliding on the film surface, it is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of 4 or more carbon atoms, an ether solvent of 8 to 12 carbon atoms or a mixture thereof. Alternatively, the aforementioned surfactant which is insoluble in water and soluble in an alkaline developer may be dissolved in an alcohol solvent of 4 or more carbon atoms, an ether solvent of 8 to 12 carbon atoms or a mixture thereof to form a material from which the protective film of the second type is formed.

After the exposure, the resist film may be subjected to PEB. PEB can be performed, for example, on a hot plate preferably at 60 to 150° C. for 1 to 5 minutes, and more preferably at 80 to 140° C. for 1 to 3 minutes.

The resist film is then developed with a developer in the form of an aqueous alkaline solution, for example, 0.1 to 5 wt %, preferably 2 to 3 wt % aqueous solution of tetramethylammonium hydroxide (TMAH) for 0.1 to 3 minutes, and preferably 0.5 to 2 minutes by conventional techniques such as dip, puddle, and spray techniques. In this way, the exposed region of the resist film is dissolved away, and a desired resist pattern is formed on the substrate.

After the resist film is formed, a step of rinsing with pure water may be introduced to extract the acid generator or the like from the film surface or wash away particles. After exposure, a step of rinsing may be introduced to remove any water remaining on the film after exposure.

Also, a double patterning process may be used for pattern formation. The double patterning process includes a trench process of processing an underlay to a 1:3 trench pattern by a first step of exposure and etching, shifting the position, and forming a 1:3 trench pattern by a second step of exposure, for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

In the pattern forming process of the present invention, negative tone development may also be used. That is, an organic solvent may be used instead of the aqueous alkaline solution as the developer for developing and dissolving away the unexposed region of the resist film.

The organic solvent used as the developer is preferably selected from 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, isopentyl acetate, butenyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, ethyl phenylacetate, benzyl formate, phenylethyl formate, methyl 3-phenylpropionate, benzyl propionate, and 2-phenylethyl acetate. These organic solvents may be used alone or in admixture.

### EXAMPLES

Synthesis Examples, Examples, and Comparative Examples are given below for specifically illustrating the present invention although the present invention is not limited thereto. The device used is as follows.
MALDI TOF-MS: S3000 by JEOL Ltd.

### [1] Synthesis of Onium Salt

### Example 1-1

### Synthesis of Onium Salt SQ-1

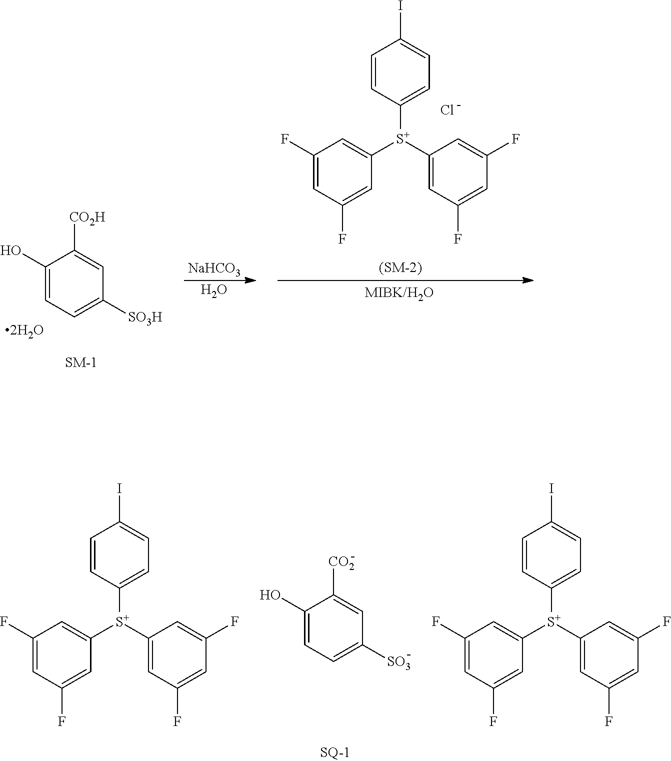

In a nitrogen atmosphere, 2.5 g of Reactant SM-1, 1.7 g of sodium hydrogencarbonate, and 30 g of water were added to a reactor, the internal temperature was raised to 50° C., and stirring was performed for 1 hour. Thereafter, the reaction solution was cooled to room temperature, 10.9 g of Reactant SM-1 and 60 g of methyl isobutyl ketone were added to the solution, and stirring was performed for 15 minutes. Thereafter, the organic layer was taken out, washed with water, and concentrated under reduced pressure. Diisopropyl ether was added to the concentrate, and the concentrate was washed to obtain target SQ-1 as oily matter (amount 9.6 g, yield 84%).

### MALDI TOF-MS:

POSITIVE M⁺461 (corresponding to C₁₈H₁₀F₄IS⁺)
NEGATIVE M⁻217 (corresponding to C₇H₅O₆S⁻)

### Examples 1-2 to 1-10

### Synthesis of Onium Salts SQ-2 to SQ-10

Onium salts SQ-2 to SQ-10 represented by the following formulae were synthesized using corresponding reactants and well-known organic synthesis reactions.

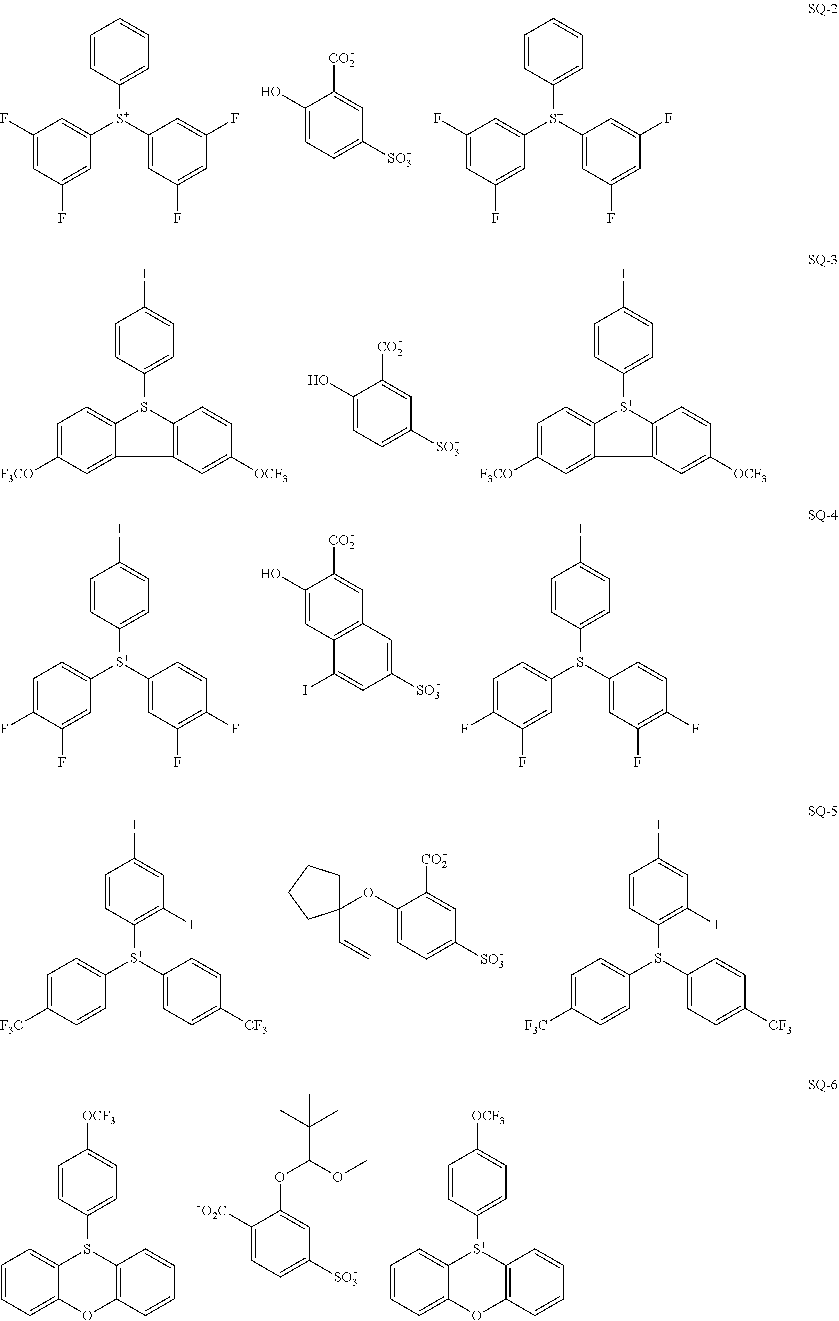

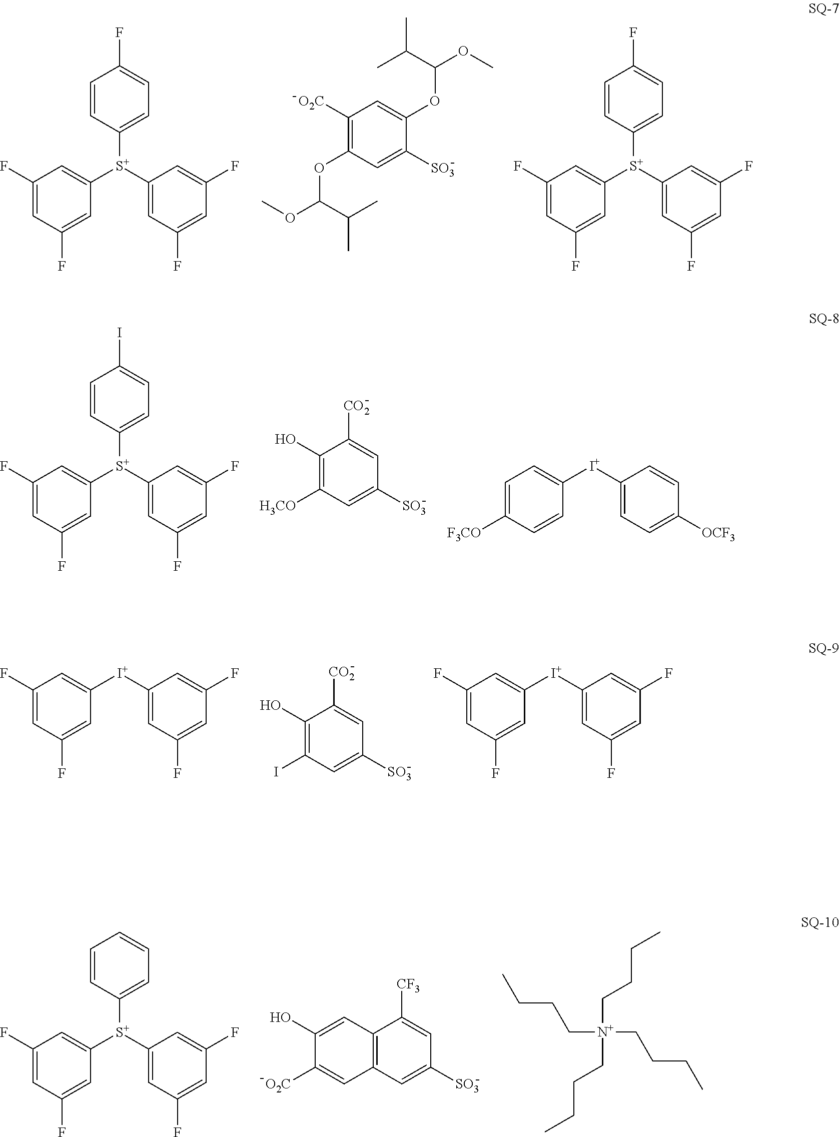

### Synthesis Example

### Synthesis of Base Polymers (P-1 to P-5)

Base polymers P-1 to P-5 were synthesized by combining monomers, performing copolymerization reaction in MEK solvent, pouring the reaction solution to hexane for precipitation, washing the solid precipitate with hexane, and performing isolation and drying. The base polymer was analyzed for composition by ¹H-NMR spectroscopy and for Mw and Mw/Mn by GPC versus polystyrene standards using THF solvent.

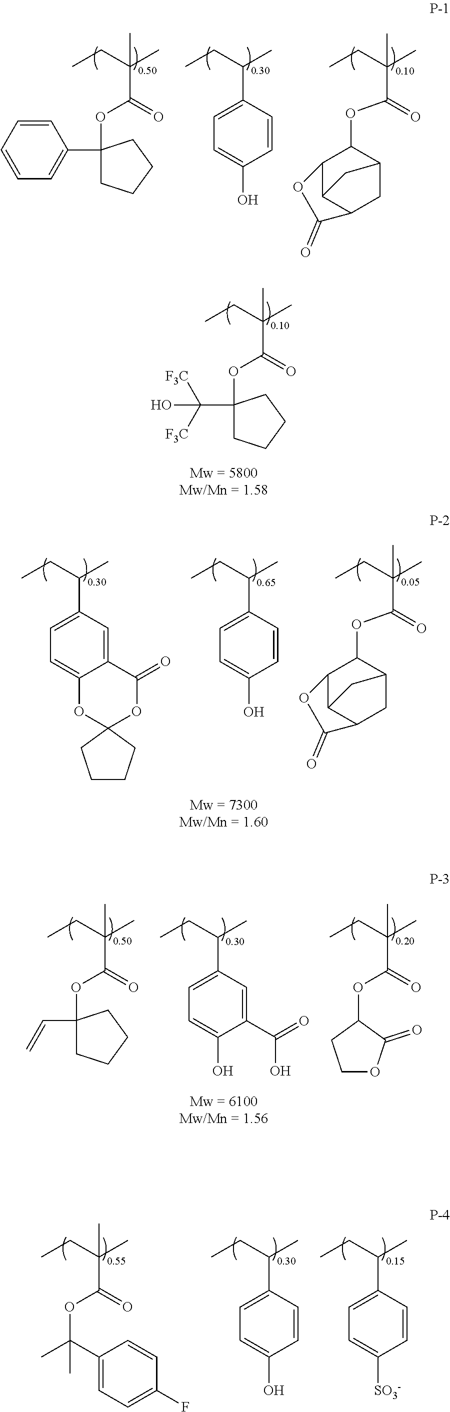

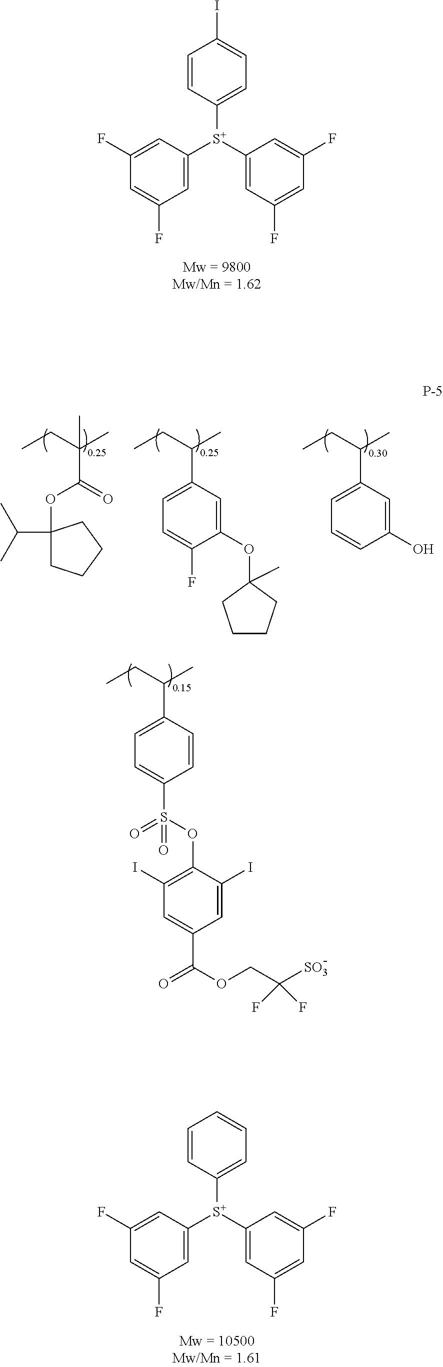

### [3] Preparation of Chemically Amplified Resist Compositions

### Examples 2-1 to 2-40 and Comparative Examples 1-1 to 1-30

Chemically amplified resist compositions (R-1 to R-40 and CR-1 to CR-30) in solution form were prepared by dissolving quenchers (SQ-1 to SQ-10) consisting of the onium salt of the present invention, comparative quenchers (SQ-A to SQ-D and AQ-A), photoacid generators (PAG-X and PAG-Y), and base polymers (P-1 to P-5) in a solvent containing 0.01 wt % of surfactant A (Omnova Solutions, Inc.) in accordance with the formulation shown in Tables 1 and 2, and filtering the solution through a Teflon® filter with a pore size of 0.2 μm.

TABLE 1

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Example | 2-1 | R-1 | P-1 (80) | SQ-1 (4.0) | PAG-X (24) | PGMEA (2250) | EL (2800) | DAA (550) |
| | 2-2 | R-2 | P-1 (80) | SQ-2 (4.0) | PAG-X (24) | PGMEA (2250) | EL (2800) | DAA (550) |
| | 2-3 | R-3 | P-1 (80) | SQ-3 (4.2) | PAG-X (24) | PGMEA (2250) | EL (2800) | DAA (550) |
| | 2-4 | R-4 | P-1 (80) | SQ-4 (4.0) | PAG-X (24) | PGMEA (2250) | EL (2800) | DAA (550) |
| | 2-5 | R-5 | P-1 (80) | SQ-5 (4.2) | PAG-X (24) | PGMEA (2250) | EL (2800) | DAA (550) |
| | 2-6 | R-6 | P-1 (80) | SQ-6 (4.0) | PAG-X (24) | PGMEA (2250) | EL (2800) | DAA (550) |
| | 2-7 | R-7 | P-1 (80) | SQ-7 (3.6) | PAG-X (22) | PGMEA (2250) | EL (2800) | DAA (550) |
| | 2-8 | R-8 | P-1 (80) | SQ-8 (3.8) | PAG-X (24) | PGMEA (2250) | EL (2800) | DAA (550) |
| | 2-9 | R-9 | P-1 (80) | SQ-9 (4.0) | PAG-X (24) | PGMEA (2250) | EL (2800) | DAA (550) |
| | 2-10 | R-10 | P-1 (80) | SQ-10 (4.0) | PAG-X (24) | PGMEA (2250) | EL (2800) | DAA (550) |
| | 2-11 | R-11 | P-1 (80) | SQ-1 (4.0) | PAG-Y (24) | PGMEA (2250) | EL (2800) | DAA (550) |
| | 2-12 | R-12 | P-1 (80) | SQ-2 (4.2) | PAG-Y (24) | PGMEA (2250) | EL (2800) | DAA (550) |
| | 2-13 | R-13 | P-1 (80) | SQ-3 (4.2) | PAG-Y (24) | PGMEA (2250) | EL (2800) | DAA (550) |
| | 2-14 | R-14 | P-1 (80) | SQ-4 (4.0) | PAG-Y (22) | PGMEA (2250) | EL (2800) | DAA (550) |
| | 2-15 | R-15 | P-1 (80) | SQ-5 (3.8) | PAG-Y (24) | PGMEA (2250) | EL (2800) | DAA (550) |
| | 2-16 | R-16 | P-1 (80) | SQ-6 (4.0) | PAG-Y (24) | PGMEA (2250) | EL (2800) | DAA (550) |
| | 2-17 | R-17 | P-1 (80) | SQ-7 (3.6) | PAG-Y (24) | PGMEA (2250) | EL (2800) | DAA (550) |
| | 2-18 | R-18 | P-1 (80) | SQ-8 (3.8) | PAG-Y (22) | PGMEA (2250) | EL (2800) | DAA (550) |
| | 2-19 | R-19 | P-1 (80) | SQ-9 (4.2) | PAG-Y (24) | PGMEA (2250) | EL (2800) | DAA (550) |
| | 2-20 | R-20 | P-1 (80) | SQ-10 (4.0) | PAG-Y (24) | PGMEA (2250) | EL (2800) | DAA (550) |
| | 2-21 | R-21 | P-2 (80) | SQ-1 (4.0) | PAG-X (22) | PGMEA (2250) | EL (2800) | DAA (550) |
| | 2-22 | R-22 | P-2 (80) | SQ-2 (4.0) | PAG-Y (24) | PGMEA (2250) | EL (2800) | DAA (550) |
| | 2-23 | R-23 | P-2 (80) | SQ-5 (4.2) | PAG-X (22) | PGMEA (2250) | EL (2800) | DAA (550) |
| | 2-24 | R-24 | P-2 (80) | SQ-6 (4.0) | PAG-Y (24) | PGMEA (2250) | EL (2800) | DAA (550) |
| | 2-25 | R-25 | P-2 (80) | SQ-7 (3.6) | PAG-X (22) | PGMEA (2250) | EL (2800) | DAA (550) |
| | 2-26 | R-26 | P-3 (80) | SQ-1 (4.0) | PAG-X (22) | PGMEA (2250) | EL (2800) | DAA (550) |
| | 2-27 | R-27 | P-3 (80) | SQ-2 (4.0) | PAG-X (22) | PGMEA (2250) | EL (2800) | DAA (550) |
| | 2-28 | R-28 | P-3 (80) | SQ-3 (4.2) | PAG-Y (24) | PGMEA (2250) | EL (2800) | DAA (550) |
| | 2-29 | R-29 | P-3 (80) | SQ-8 (3.8) | PAG-X (22) | PGMEA (2250) | EL (2800) | DAA (550) |
| | 2-30 | R-30 | P-3 (80) | SQ-9 (4.2) | PAG-Y (24) | PGMEA (2250) | EL (2800) | DAA (550) |
| | 2-31 | R-31 | P-4 (80) | SQ-1 (4.0) | — | PGMEA (2250) | EL (2800) | DAA (550) |
| | 2-32 | R-32 | P-4 (80) | SQ-2 (4.0) | — | PGMEA (2250) | EL (2800) | DAA (550) |
| | 2-33 | R-33 | P-4 (80) | SQ-4 (4.0) | — | PGMEA (2250) | EL (2800) | DAA (550) |
| | 2-34 | R-34 | P-4 (80) | SQ-7 (3.6) | — | PGMEA (2250) | EL (2800) | DAA (550) |
| | 2-35 | R-35 | P-4 (80) | SQ-10 (4.0) | PAG-Y (12) | PGMEA (2250) | EL (2800) | DAA (550) |
| | 2-36 | R-36 | P-5 (80) | SQ-1 (4.0) | — | PGMEA (2250) | EL (2800) | DAA (550) |
| | 2-37 | R-37 | P-5 (80) | SQ-2 (4.0) | — | PGMEA (2250) | EL (2800) | DAA (550) |
| | 2-38 | R-38 | P-5 (80) | SQ-5 (4.2) | — | PGMEA (2250) | EL (2800) | DAA (550) |
| | 2-39 | R-39 | P-5 (80) | SQ-7 (3.6) | — | PGMEA (2250) | EL (2800) | DAA (550) |
| | 2-40 | R-40 | P-5 (80) | SQ-9 (4.2) | PAG-X (8) | PGMEA (2250) | EL (2800) | DAA (550) |

TABLE 2

| | | | Base | | Photoacid | | | |
|---|---|---|---|---|---|---|---|---|
| | | Resist | polymer | Quencher | generator | Solvent 1 | Solvent 2 | Solvent 3 |
| | | composition | (pbw) | (pbw) | (pbw) | (pbw) | (pbw) | (pbw) |
| Comparative | 1-1 | CR-1 | P-1 (80) | SQ-A (8.0) | PAG-X (24) | PGMEA (2250) | EL (2800) | DAA (550) |
| Example | 1-2 | CR-2 | P-1 (80) | SQ-B (8.2) | PAG-X (24) | PGMEA (2250) | EL (2800) | DAA (550) |
| | 1-3 | CR-3 | P-1 (80) | SQ-C (8.0) | PAG-X (24) | PGMEA (2250) | EL (2800) | DAA (550) |
| | 1-4 | CR-4 | P-1 (80) | SQ-D (7.8) | PAG-X (24) | PGMEA (2250) | EL (2800) | DAA (550) |
| | 1-5 | CR-5 | P-1 (80) | SQ-A (8.0) | PAG-Y (24) | PGMEA (2250) | EL (2800) | DAA (550) |
| | 1-6 | CR-6 | P-1 (80) | SQ-B (7.6) | PAG-Y (24) | PGMEA (2250) | EL (2800) | DAA (550) |
| | 1-7 | CR-7 | P-1 (80) | SQ-C (8.2) | PAG-Y (24) | PGMEA (2250) | EL (2800) | DAA (550) |
| | 1-8 | CR-8 | P-1 (80) | SQ-D (7.8) | PAG-Y (24) | PGMEA (2250) | EL (2800) | DAA (550) |
| | 1-9 | CR-9 | P-1 (80) | SQ-A (4.0) | PAG-X (22) | PGMEA (2250) | EL (2800) | DAA (550) |
| | | | | SQ-C (3.6) | | | | |
| | 1-10 | CR-10 | P-1 (80) | SQ-B (4.0) | PAG-Y (24) | PGMEA (2250) | EL (2800) | DAA (550) |
| | | | | SQ-D (3.6) | | | | |
| | 1-11 | CR-11 | P-1 (80) | SQ-A (4.0) | PAG-Y (24) | PGMEA (2250) | EL (2800) | DAA (550) |
| | | | | AQ-A (3.6) | | | | |
| | 1-12 | CR-12 | P-2 (80) | SQ-A (8.0) | PAG-X (24) | PGMEA (2250) | EL (2800) | DAA (550) |
| | 1-13 | CR-13 | P-2 (80) | SQ-B (8.2) | PAG-Y (24) | PGMEA (2250) | EL (2800) | DAA (550) |
| | 1-14 | CR-14 | P-2 (80) | SQ-A (4.0) | PAG-X (22) | PGMEA (2250) | EL (2800) | DAA (550) |
| | | | | SQ-C (3.6) | | | | |
| | 1-15 | CR-15 | P-3 (80) | SQ-A (8.2) | PAG-Y (24) | PGMEA (2250) | EL (2800) | DAA (550) |
| | 1-16 | CR-16 | P-3 (80) | SQ-C (8.0) | PAG-X (20) | PGMEA (2250) | EL (2800) | DAA (550) |
| | 1-17 | CR-17 | P-3 (80) | SQ-B (4.0) | PAG-Y (24) | PGMEA (2250) | EL (2800) | DAA (550) |
| | | | | SQ-C (3.6) | | | | |
| | 1-18 | CR-18 | P-4 (80) | SQ-A (8.2) | — | PGMEA (2250) | EL (2800) | DAA (550) |
| | 1-19 | CR-19 | P-4 (80) | SQ-C (7.6) | — | PGMEA (2250) | EL (2800) | DAA (550) |
| | 1-20 | CR-20 | P-4 (80) | SQ-A (4.0) | PAG-Y (12) | PGMEA (2250) | EL (2800) | DAA (550) |
| | | | | SQ-D (3.6) | | | | |
| | 1-21 | CR-21 | P-5 (80) | SQ-A (8.2) | — | PGMEA (2250) | EL (2800) | DAA (550) |
| | 1-22 | CR-22 | P-5 (80) | SQ-B (7.8) | — | PGMEA (2250) | EL (2800) | DAA (550) |
| | 1-23 | CR-23 | P-5 (80) | SQ-C (8.0) | — | PGMEA (2250) | EL (2800) | DAA (550) |
| | 1-24 | CR-24 | P-5 (80) | SQ-D (8.2) | — | PGMEA (2250) | EL (2800) | DAA (550) |
| | 1-25 | CR-25 | P-5 (80) | AQ-A (8.0) | — | PGMEA (2250) | EL (2800) | DAA (550) |
| | 1-26 | CR-26 | P-5 (80) | SQ-D (4.8) | PAG-X (24) | PGMEA (2250) | EL (2800) | DAA (550) |
| | | | | AQ-A (3.6) | | | | |
| | 1-27 | CR-27 | P-5 (80) | SQ-A (8.2) | PAG-X (8) | PGMEA (2250) | EL (2800) | DAA (550) |
| | 1-28 | CR-28 | P-5 (80) | SQ-A (4.0) | — | PGMEA (2250) | EL (2800) | DAA (550) |
| | | | | SQ-C (3.6) | | | | |
| | 1-29 | CR-29 | P-5 (80) | SQ-B (4.0) | — | PGMEA (2250) | EL (2800) | DAA (550) |
| | | | | SQ-D (3.6) | | | | |
| | 1-30 | CR-30 | P-5 (80) | SQ-B (4.0) | — | PGMEA (2250) | EL (2800) | DAA (550) |
| | | | | SQ-C (3.6) | | | | |

The solvents, photoacid generators PAG-X and PAG-Y, comparative quenchers SQ-A to SQ-D and AQ-A, and surfactant A in Tables 1 and 2 are identified below.

### Solvent:

PGMEA (propylene glycol monomethyl ether acetate)
EL (ethyl lactate)
DAA (diacetone alcohol)

### Photoacid Generators: PAG-X and PAG-Y

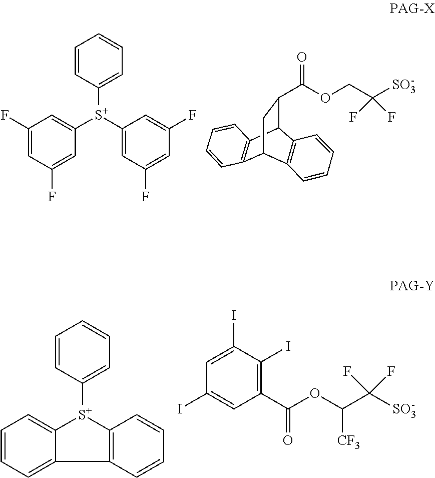

### Comparative Quenchers: SQ-A to SQ-D and AQ-A

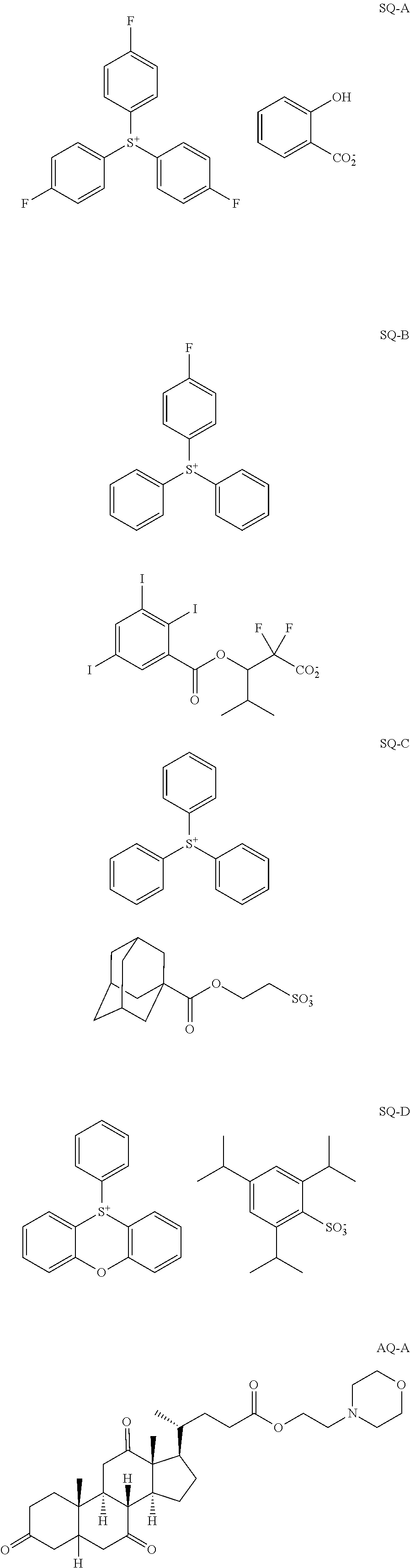

### Surfactant A:

3-methyl-3-(2,2,2-trifluoroethoxymethyl) oxetane/tetrahydrofuran/
2,2-dimethyl-1,3-propanediol copolymer (Omnova Solutions, Inc.)

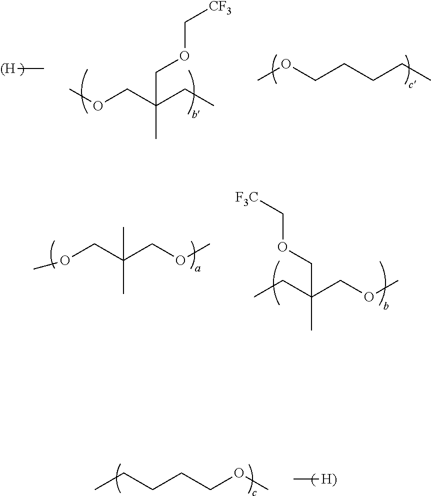

a:(b+b′):(c+c′)=1:4-7:0.01-1 (molar ratio)
Mw=1,500

### [4] EUV Lithography Test (1)

### Examples 3-1 to 3-40 and Comparative Examples 2-1 to 2-30

Each of the chemically amplified resist compositions (R-1 to R-40 and CR-1 to CR-30) was spin coated on a silicon substrate having a 20-nm coating of silicon-containing spin-on hard mask SHB-A940 (Shin-Etsu Chemical Co., Ltd., silicon content 43 wt %) and prebaked on a hot plate at 100° C. for 60 seconds to form a resist film of 50 nm thick. Using an EUV scanner NXE3400 (ASML, NA 0.33, σ 0.9/0.6, dipole illumination), the resist film was exposed to EUV through a mask bearing a LS pattern having a size of 18 nm and a pitch of 36 nm (on-wafer size) while varying the dose and focus (dose pitch: 1 mJ/cm², focus pitch: 0.020 μm), and after exposure, the resist film was baked (PEB) at room temperature shown in Tables 4 and 5 for 60 seconds. Thereafter, the resist film was puddle developed in a 2.38 wt % TMAH aqueous solution for 30 seconds, rinsed with a surfactant-containing rinse fluid, and spin dried to form a positive pattern.

The obtained LS pattern was observed under CD-SEM (CG6300, Hitachi High-Technologies Corp.) whereupon sensitivity, EL, LWR, DOF, and collapse limit were evaluated by the following methods. The results are shown in Tables 3 and 4.

### [Evaluation of Sensitivity]

The optimum dose Eₒₚ (mJ/cm²) which provided a LS pattern with a line width of 18 nm and a pitch of 36 nm was determined as an index of sensitivity. A smaller value indicates a higher sensitivity.

### [Evaluation of EL]

The exposure dose which provided a LS pattern with a space width of 18 nm±10% (i.e., 16.2 to 19.8 nm) was determined. EL (unit: %) was calculated from the exposure doses according to the following equation. A greater value indicates better performance.

$$EL ( \% ) = ( \vert {E}_{1} - {E}_{2} \vert / Eop ) \times 100$$

E₁: optimum exposure dose which provides LS pattern with line width of 16.2 nm and pitch of 36 nm
E₂: optimum exposure dose which provides LS pattern with line width of 19.8 nm and pitch of 36 nm
Eₒₚ: optimum exposure dose which provides LS pattern with line width of 18 nm and pitch of 36 nm

### [Evaluation of LWR]

For the LS pattern formed by exposure at the optimum dose Eop, the line width was measured at 10 longitudinally spaced apart points, from which a 3-fold value (30) of the standard deviation (σ) was determined and reported as LWR. A smaller value of 30 indicates a pattern having small roughness and uniform line width.

### [Evaluation of DOF]

As an index of DOF, a range of focus which provided a LS pattern with a size of 18 nm±10% (i.e., 16.2 to 19.8 nm) was determined. A greater value indicates a wider DOF.

### [Evaluation of Collapse Limit of Line Pattern]

For the LS pattern formed by exposure at the dose corresponding to the optimum focus, the line width was measured at 10 longitudinally spaced apart points. The minimum line size above which lines could be resolved without collapse was determined and reported as collapse limit. A smaller value indicates better collapse limit.

TABLE 3

| | | Resist | PEB temp. | Eop | EL | LWR | DOF | Collapse limit |
|---|---|---|---|---|---|---|---|---|
| | | composition | (° C.) | (mJ/cm²) | (%) | (nm) | (nm) | (nm) |
| Example | 3-1 | R-1 | 95 | 31 | 18 | 2.2 | 120 | 10.7 |
| | 3-2 | R-2 | 100 | 33 | 19 | 2.4 | 110 | 10.8 |
| | 3-3 | R-3 | 100 | 32 | 17 | 2.3 | 110 | 11.2 |
| | 3-4 | R-4 | 95 | 32 | 18 | 2.5 | 110 | 10.8 |
| | 3-5 | R-5 | 105 | 33 | 17 | 2.4 | 110 | 11.2 |
| | 3-6 | R-6 | 100 | 31 | 18 | 2.4 | 100 | 11.3 |
| | 3-7 | R-7 | 95 | 32 | 18 | 2.3 | 110 | 10.8 |
| | 3-8 | R-8 | 95 | 33 | 17 | 2.2 | 120 | 10.9 |
| | 3-9 | R-9 | 100 | 32 | 16 | 2.4 | 110 | 11.2 |
| | 3-10 | R-10 | 100 | 32 | 19 | 2.5 | 110 | 11.3 |
| | 3-11 | R-11 | 100 | 33 | 17 | 2.4 | 110 | 11.2 |
| | 3-12 | R-12 | 95 | 34 | 18 | 2.3 | 100 | 11.0 |
| | 3-13 | R-13 | 105 | 32 | 17 | 2.3 | 110 | 10.8 |
| | 3-14 | R-14 | 100 | 34 | 18 | 2.4 | 120 | 10.8 |
| | 3-15 | R-15 | 95 | 33 | 18 | 2.2 | 110 | 10.9 |
| | 3-16 | R-16 | 95 | 33 | 17 | 2.3 | 110 | 10.8 |
| | 3-17 | R-17 | 100 | 32 | 17 | 2.5 | 120 | 11.1 |
| | 3-18 | R-18 | 95 | 31 | 16 | 2.4 | 120 | 11.3 |
| | 3-19 | R-19 | 95 | 32 | 17 | 2.5 | 110 | 11.5 |
| | 3-20 | R-20 | 100 | 33 | 18 | 2.4 | 120 | 11.2 |
| | 3-21 | R-21 | 100 | 32 | 17 | 2.2 | 110 | 10.9 |
| | 3-22 | R-22 | 100 | 34 | 19 | 2.3 | 120 | 10.7 |
| | 3-23 | R-23 | 95 | 31 | 18 | 2.3 | 100 | 11.1 |
| | 3-24 | R-24 | 95 | 32 | 17 | 2.4 | 110 | 11.2 |
| | 3-25 | R-25 | 100 | 34 | 17 | 2.3 | 100 | 10.7 |
| | 3-26 | R-26 | 100 | 33 | 17 | 2.5 | 110 | 10.9 |
| | 3-27 | R-27 | 100 | 31 | 18 | 2.4 | 120 | 10.7 |
| | 3-28 | R-28 | 95 | 32 | 17 | 2.3 | 110 | 11.2 |
| | 3-29 | R-29 | 95 | 32 | 19 | 2.2 | 100 | 10.9 |
| | 3-30 | R-30 | 100 | 33 | 18 | 2.4 | 120 | 11.1 |
| | 3-31 | R-31 | 100 | 33 | 17 | 2.3 | 110 | 11.3 |
| | 3-32 | R-32 | 100 | 35 | 18 | 2.3 | 110 | 11.2 |
| | 3-33 | R-33 | 95 | 34 | 18 | 2.4 | 100 | 11.1 |
| | 3-34 | R-34 | 95 | 33 | 17 | 2.5 | 120 | 11.0 |
| | 3-35 | R-35 | 100 | 33 | 17 | 2.5 | 110 | 10.9 |
| | 3-36 | R-36 | 100 | 32 | 17 | 2.4 | 110 | 11.1 |
| | 3-37 | R-37 | 100 | 34 | 18 | 2.2 | 120 | 10.8 |
| | 3-38 | R-38 | 95 | 33 | 17 | 2.3 | 110 | 11.0 |
| | 3-39 | R-39 | 95 | 33 | 19 | 2.4 | 100 | 10.9 |
| | 3-40 | R-40 | 100 | 32 | 18 | 2.3 | 120 | 10.8 |

TABLE 4

| | | Resist | PEB temp. | Eop | EL | LWR | DOF | Collapse limit |
|---|---|---|---|---|---|---|---|---|
| | | composition | (° C.) | (mJ/cm²) | (%) | (nm) | (nm) | (nm) |
| Comparative | 2-1 | CR-1 | 95 | 37 | 14 | 2.7 | 90 | 11.5 |
| Example | 2-2 | CR-2 | 100 | 38 | 13 | 2.9 | 80 | 11.6 |
| | 2-3 | CR-3 | 100 | 38 | 14 | 3.0 | 90 | 12.4 |
| | 2-4 | CR-4 | 100 | 39 | 12 | 3.2 | 70 | 12.8 |
| | 2-5 | CR-5 | 95 | 37 | 15 | 2.8 | 90 | 11.9 |
| | 2-6 | CR-6 | 100 | 38 | 14 | 2.9 | 80 | 11.8 |
| | 2-7 | CR-7 | 100 | 38 | 13 | 2.9 | 70 | 11.9 |
| | 2-8 | CR-8 | 100 | 39 | 12 | 3.1 | 70 | 12.2 |
| | 2-9 | CR-9 | 95 | 40 | 14 | 2.9 | 90 | 12.3 |
| | 2-10 | CR-10 | 100 | 39 | 13 | 2.8 | 80 | 11.9 |
| | 2-11 | CR-11 | 100 | 40 | 14 | 2.9 | 90 | 12.1 |
| | 2-12 | CR-12 | 100 | 37 | 15 | 3.0 | 80 | 12.4 |
| | 2-13 | CR-13 | 95 | 38 | 12 | 3.1 | 90 | 12.2 |
| | 2-14 | CR-14 | 105 | 39 | 13 | 3.2 | 100 | 11.9 |
| | 2-15 | CR-15 | 100 | 38 | 14 | 2.9 | 80 | 12.4 |
| | 2-16 | CR-16 | 95 | 38 | 13 | 2.7 | 90 | 12.2 |
| | 2-17 | CR-17 | 95 | 39 | 13 | 2.7 | 70 | 12.2 |
| | 2-18 | CR-18 | 100 | 37 | 15 | 2.8 | 90 | 12.3 |
| | 2-19 | CR-19 | 95 | 40 | 14 | 3.1 | 80 | 11.9 |
| | 2-20 | CR-20 | 95 | 39 | 14 | 2.9 | 90 | 12.1 |
| | 2-21 | CR-21 | 100 | 40 | 13 | 2.8 | 80 | 11.7 |
| | 2-22 | CR-22 | 100 | 39 | 12 | 3.0 | 90 | 11.8 |
| | 2-23 | CR-23 | 100 | 38 | 14 | 2.2 | 70 | 12.0 |
| | 2-24 | CR-24 | 100 | 39 | 14 | 2.9 | 80 | 12.4 |
| | 2-25 | CR-25 | 95 | 38 | 12 | 2.8 | 80 | 11.9 |
| | 2-26 | CR-26 | 95 | 39 | 13 | 2.9 | 90 | 12.1 |
| | 2-27 | CR-27 | 95 | 38 | 14 | 2.8 | 80 | 12.3 |
| | 2-28 | CR-28 | 95 | 39 | 13 | 2.7 | 80 | 11.9 |
| | 2-29 | CR-29 | 95 | 38 | 13 | 2.9 | 90 | 12.3 |
| | 2-30 | CR-30 | 95 | 38 | 14 | 3.0 | 70 | 12.1 |

From the results shown in Tables 3 and 4, it was found that the chemically amplified resist composition of the present invention comprising a quencher consisting of the onium salt of the present invention has a high sensitivity and improved values of EL, LWR, and DOF. Small values of collapse limit attest that in forming a small-size pattern, the pattern is resistant to collapse. The chemically amplified resist compositions of the present invention are useful in the EUV lithography process.

### [5] EUV Lithography Test (2)

### Examples 4-1 to 4-40 and Comparative Examples 3-1 to 3-30

Each of the chemically amplified resist compositions (R-1 to R-40 and CR-1 to CR-30) was spin coated on a silicon substrate having a 20-nm coating of silicon-containing spin-on hard mask SHB-A940 (Shin-Etsu Chemical Co., Ltd., silicon content 43 wt %) and prebaked on a hot plate at 105° C. for 60 seconds to form a resist film of 50 nm thick. Using an EUV scanner NXE3400 (ASML, NA 0.33, σ 0.9/0.6, quadrupole illumination), the resist film was exposed to EUV through a mask bearing a hole pattern having a pitch of 46 nm +20% bias (on-wafer size). The resist film was baked (PEB) on a hot plate at the temperature shown in Tables 5 and 6 for 60 seconds and developed in a 2.38 wt % TMAH aqueous solution for 30 seconds to form a hole pattern having a size of 23 nm.

The pattern as developed was observed under CD-SEM (CG6300, Hitachi High-Technologies Corp.). The dose at which a pattern with a hole size of 23 nm was printed was determined as an index of sensitivity. The size of 50 holes was measured, from which a 3-fold value (36) of the standard deviation (6) was determined as CDU. The results are shown in Tables 5 and 6.

TABLE 5

| | | Resist | PEB temp. | Eop | CDU |
|---|---|---|---|---|---|
| | | composition | (° C.) | (mJ/cm²) | (nm) |
| Example | 4-1 | R-1 | 90 | 22 | 2.3 |
| | 4-2 | R-2 | 95 | 23 | 2.2 |
| | 4-3 | R-3 | 95 | 24 | 2.3 |
| | 4-4 | R-4 | 90 | 23 | 2.4 |
| | 4-5 | R-5 | 90 | 24 | 2.5 |
| | 4-6 | R-6 | 95 | 25 | 2.4 |
| | 4-7 | R-7 | 90 | 23 | 2.3 |
| | 4-8 | R-8 | 95 | 22 | 2.2 |
| | 4-9 | R-9 | 95 | 24 | 2.4 |
| | 4-10 | R-10 | 90 | 23 | 2.5 |
| | 4-11 | R-11 | 95 | 22 | 2.4 |
| | 4-12 | R-12 | 95 | 24 | 2.5 |
| | 4-13 | R-13 | 90 | 25 | 2.4 |
| | 4-14 | R-14 | 90 | 23 | 2.3 |
| | 4-15 | R-15 | 90 | 23 | 2.3 |
| | 4-16 | R-16 | 85 | 24 | 2.4 |
| | 4-17 | R-17 | 95 | 23 | 2.3 |
| | 4-18 | R-18 | 95 | 22 | 2.5 |
| | 4-19 | R-19 | 95 | 24 | 2.3 |
| | 4-20 | R-20 | 95 | 23 | 2.2 |
| | 4-21 | R-21 | 95 | 24 | 2.4 |
| | 4-22 | R-22 | 95 | 22 | 2.2 |
| | 4-23 | R-23 | 95 | 23 | 2.3 |
| | 4-24 | R-24 | 90 | 23 | 2.3 |
| | 4-25 | R-25 | 95 | 25 | 2.5 |
| | 4-26 | R-26 | 95 | 23 | 2.3 |
| | 4-27 | R-27 | 95 | 24 | 2.4 |
| | 4-28 | R-28 | 95 | 25 | 2.3 |
| | 4-29 | R-29 | 90 | 24 | 2.2 |
| | 4-30 | R-30 | 90 | 25 | 2.4 |
| | 4-31 | R-31 | 95 | 23 | 2.5 |
| | 4-32 | R-32 | 90 | 22 | 2.3 |
| | 4-33 | R-33 | 95 | 24 | 2.4 |
| | 4-34 | R-34 | 95 | 23 | 2.2 |
| | 4-35 | R-35 | 95 | 23 | 2.4 |
| | 4-36 | R-36 | 95 | 24 | 2.3 |
| | 4-37 | R-37 | 95 | 25 | 2.2 |
| | 4-38 | R-38 | 90 | 23 | 2.5 |
| | 4-39 | R-39 | 95 | 22 | 2.4 |
| | 4-40 | R-40 | 90 | 24 | 2.4 |

TABLE 6

| | | Resist | PEB temp. | Eop | CDU |
|---|---|---|---|---|---|
| | | composition | (° C.) | (mJ/cm²) | (nm) |
| Comparative | 3-1 | CR-1 | 95 | 28 | 2.8 |
| Example | 3-2 | CR-2 | 95 | 29 | 2.9 |
| | 3-3 | CR-3 | 95 | 28 | 2.7 |
| | 3-4 | CR-4 | 90 | 27 | 3.1 |
| | 3-5 | CR-5 | 95 | 29 | 2.9 |
| | 3-6 | CR-6 | 95 | 30 | 2.8 |
| | 3-7 | CR-7 | 90 | 28 | 2.9 |
| | 3-8 | CR-8 | 90 | 29 | 3.1 |
| | 3-9 | CR-9 | 90 | 28 | 2.9 |
| | 3-10 | CR-10 | 95 | 29 | 2.8 |
| | 3-11 | CR-11 | 95 | 30 | 2.9 |
| | 3-12 | CR-12 | 95 | 28 | 2.8 |
| | 3-13 | CR-13 | 90 | 29 | 2.9 |
| | 3-14 | CR-14 | 95 | 27 | 3.0 |
| | 3-15 | CR-15 | 95 | 29 | 2.8 |
| | 3-16 | CR-16 | 95 | 28 | 2.9 |
| | 3-17 | CR-17 | 95 | 29 | 2.9 |
| | 3-18 | CR-18 | 95 | 28 | 2.8 |
| | 3-19 | CR-19 | 90 | 29 | 2.8 |
| | 3-20 | CR-20 | 95 | 30 | 3.0 |
| | 3-21 | CR-21 | 95 | 28 | 2.9 |
| | 3-22 | CR-22 | 90 | 29 | 3.1 |
| | 3-23 | CR-23 | 90 | 27 | 2.8 |
| | 3-24 | CR-24 | 95 | 29 | 2.9 |
| | 3-25 | CR-25 | 95 | 30 | 3.1 |
| | 3-26 | CR-26 | 95 | 29 | 2.8 |
| | 3-27 | CR-27 | 95 | 30 | 2.7 |
| | 3-28 | CR-28 | 95 | 29 | 2.8 |
| | 3-29 | CR-29 | 95 | 29 | 2.8 |
| | 3-30 | CR-30 | 95 | 30 | 2.9 |

From the results shown in Tables 5 and 6, it was confirmed that the chemically amplified resist composition of the present invention comprising a quencher consisting of the onium salt of the present invention has a high sensitivity and an improved value of CDU.

Japanese Patent Application No. 2024-118718 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. An onium salt having the formula (1): 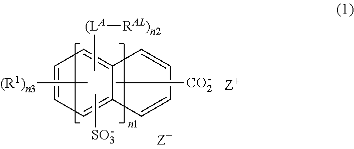
wherein n1 is 0 or 1, n2 is 1, 2, or 3, n3 is 0, 1, or 2, provided that 1≤n2+n3≤4 when n1 is 0, and 1≤n2+n3≤6 when n1 is 1,
R¹ is a halogen atom, a nitro group, a cyano group, a pentafluorosulfanyl group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when n3 is 2, two R¹ may be identical or different, and two R¹ may bond together to form a ring with the carbon atoms to which they are attached,
R^{AL} is a hydrogen atom or an acid labile group,
L^{A} is an oxygen atom when R^{AL} is a hydrogen atom, and is an ether bond, an ester bond, or a carbonate bond when R^{AL} is an acid labile group, and
Z⁺ is each independently an onium cation.

2. The onium salt of claim 1 wherein R^{AL} is an acid labile group having the formula (AL-1) or (AL-2): 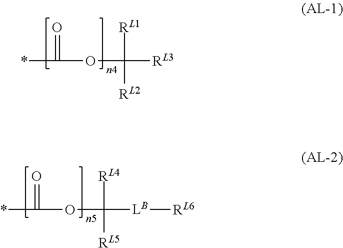
wherein n4 is 0 or 1, n5 is 0 or 1,
R^{L1}, R^{L2}, and R^{L3} are each independently a C₁-C₁₂ hydrocarbyl group in which some —CH₂— may be replaced by —O— or —S—, when the hydrocarbyl group contains an aromatic ring, some or all of the hydrogen atoms on the aromatic ring may be substituted by a halogen atom, a cyano group, a nitro group, an optionally halogenated C₁-C₄ alkyl group, or an optionally halogenated C₁-C₄ alkoxy group, R^{L1} and R^{L2} may bond together to form a ring with the carbon atom to which they are attached, some —CH₂— in the ring may be replaced by —O— or —S—,
R^{L4} and R^{L5} are each independently a hydrogen atom or a C₁-C₁₀ hydrocarbyl group, R^{L6} is a C₁-C₂₀ hydrocarbyl group in which some —CH₂— may be replaced by —O— or —S—, R^{L5} and R^{L6} may bond together to form a C₃-C₂₀ heterocyclic group with the carbon atom and L^{B} to which they are attached, some —CH₂— in the heterocyclic group may be replaced by —O— or —S—,
L^{B} is-O— or —S—, and
* designates a point of attachment to the adjacent —O—.

3. The onium salt of claim 1 having the formula (1A): 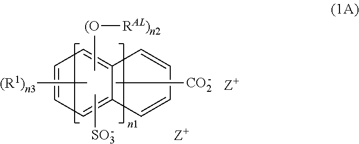
wherein R¹, R^{AL}, n1 to n3, and Z⁺ are as defined above.

4. The onium salt of claim 3 having the formula (1B): 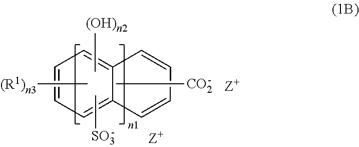
wherein R¹, n1 to n3, and Z⁺ are as defined above.

5. The onium salt of claim 1 wherein Z⁺ is a sulfonium cation having the formula (Z-1), an iodonium cation having the formula (Z-2), or an ammonium cation having the formula (Z-3): 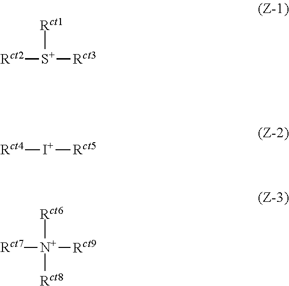
wherein R^{ct1} to R^{ct9} are each independently a halogen atom or a C₁-C₃₀ hydrocarbyl group which may contain a heteroatom, R^{ct1} and R^{ct2} may bond together to form a ring together with the sulfur atom to which they are attached, and any two of R^{ct6} to R^{ct9} may be bond together to form a ring together with the nitrogen atom to which they are attached.

6. The onium salt of claim 1 wherein Z⁺ is a sulfonium cation having the formula (Z-4): 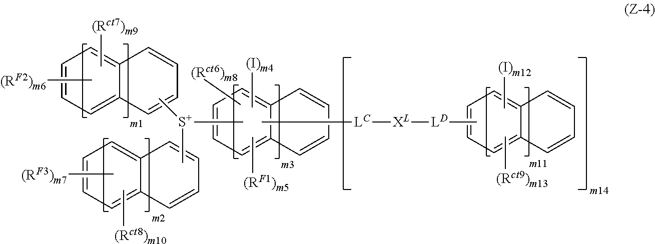
wherein m1 is 0 or 1, m2 is 0 or 1, m3 is 0 or 1, m4 is 0, 1, 2, 3, or 4, m5 is 0, 1, 2, 3, or 4, m6 is 0, 1, 2, 3, 4, 5, or 6, m7 is 0, 1, 2, 3, 4, 5, or 6, m8 is 0, 1, or 2, m9 is 0, 1, or 2, m10 is 0, 1, or 2, m11 is 0 or 1, m12 is 0, 1, 2, 3, or 4, m13 is 0, 1, or 2, m14 is 0, 1, or 2, provided that 0≤m6+m9≤4 when m1 is 0 and 0≤m6+m9≤6 when m1 is 1, 0≤m7+m10≤4 when m2 is 0 and 0≤m7+m10≤6 when m2 is 1, 1≤m4+m5+m8+m14≤4 when m3 is 0 and 1≤m4+m5+m8+m14≤6 when m3 is 1, 0≤m12+m13≤4 when m11 is 0 and 0≤m12+m13≤6 when m11 is 1, and m4+m12≥1,
R^{F1} to R^{F3} are each independently a fluorine atom, a C₁-C₆ fluorinated saturated hydrocarbyl group, a C₁-C₆ fluorinated saturated hydrocarbyloxy group, or a C₁-C₆ fluorinated saturated hydrocarbylthio group, a plurality of R^{F1} may be identical or different when m5 is 2 or more, a plurality of R^{F2} may be identical or different when m6 is 2 or more, a plurality of R^{F3} may be identical or different when m7 is 2 or more,
R^{ct6} to R^{ct9} each are a halogen atom other than an iodine atom and a fluorine atom, a nitro group, a cyano group, a C₁-C₂₀ hydrocarbyl group which may contain a heteroatom, a C₁-C₂₀ hydrocarbyloxy group which may contain a heteroatom, or a C₁-C₂₀ hydrocarbylthio group which may contain a heteroatom, when m8 is 2, two R^{ct6} may be identical or different and two R^{ct6} may bond together to form a ring with the carbon atoms to which they are attached, when m9 is 2, two R^{ct7} may be identical or different and two R^{ct7} may bond together to form a ring with the carbon atoms to which they are attached, when m10 is 2, two R^{ct8} may be identical or different and two R^{ct8} may bond together to form a ring with the carbon atoms to which they are attached, and when m13 is 2, two R^{ct9} may be identical or different and two R^{ct9} may bond together to form a ring with the carbon atoms to which they are attached,
the aromatic rings directly bonded to S⁺ in the sulfonium cation may bond together to form a ring with S⁺,
L^{C} and L^{D} are each independently a single bond, an ether bond, an ester bond, an amide bond, a sulfonate ester bond, a sulfonamide bond, a carbonate bond, or a carbamate bond, and
X^{L} is a single bond or a C₁-C₄₀ hydrocarbylene group which may contain a heteroatom.

7. The onium salt of claim 6 wherein the sulfonium cation having formula (Z-4) has the formula (Z-4-1): 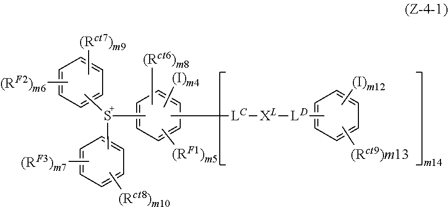
wherein m4 to m10, m12 to m14, R^{F1} to R^{F3}, R^{ct6} to R^{ct9}, L^{C}, L^{D}, and X^{L} are as defined above.

8. The onium salt of claim 7 wherein the sulfonium cation having formula (Z-4-1) has the formula (Z-4-2): 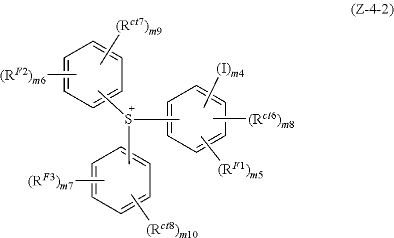
wherein m4 to m10, R^{F1} to R^{F3}, and R^{ct6} to R^{ct8} are as defined above.

9. A quencher comprising the onium salt of claim 1.

10. A chemically amplified resist composition comprising the quencher of claim 9.

11. The chemically amplified resist composition of claim 10, further comprising a base polymer comprising repeat units of at least one type selected from repeat units having the formula (a1), repeat units having the formula (a2), and repeat units having the formula (a3): 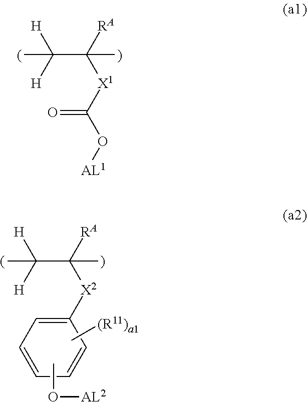
wherein R^{A} is each independently a hydrogen atom, a fluorine atom, a methyl group, or a trifluoromethyl group,
X¹ is a single bond, a phenylene group, a naphthylene group, *—C(═O)—O—X¹¹—, or *—C(═O)—NH—X¹¹—, the phenylene group or the naphthylene group may be substituted by a hydroxy group, a nitro group, a cyano group, an optionally fluorinated C₁-C₁₀ saturated hydrocarbyl group, an optionally fluorinated C₁-C₁₀ saturated hydrocarbyloxy group, or a halogen atom, X¹¹ is a C₁-C₁₀ saturated hydrocarbylene group, a phenylene group, or a naphthylene group which may contain a hydroxy group, an ether bond, an ester bond, or a lactone ring,
X² is a single bond, *—C(═O)—O—, or *—C(═O)—NH—,
* designates a point of attachment to the carbon atom in the backbone,
R¹¹ is a halogen atom, a cyano group, a hydroxy group, a nitro group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R¹¹ may be identical or different when a1 is 2, 3, or 4,
AL¹ and AL² are each independently an acid labile group,
a1 is 0, 1, 2, 3, or 4, 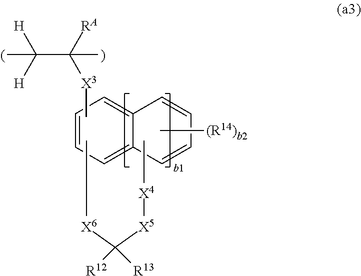
wherein b1 is 0 or 1, b2 is 0, 1, 2, or 3 when b1 is 0, and is 0, 1, 2, 3, 4, or 5 when b1 is 1,
R^{A} is a hydrogen atom, a fluorine atom, a methyl group, or a trifluoromethyl group,
X³ is a single bond, *—C(═O)—O—, or *—C(═O)—NH—, * designates a point of attachment to the carbon atom in the backbone,
X⁴ is a single bond, a C₁-C₄ aliphatic hydrocarbylene group, a carbonyl group, a sulfonyl group, or a group obtained by combining the foregoing,
X⁵ and X⁶ are each independently an oxygen atom or a sulfur atom, provided that X⁴ and X⁶ are bonded to vicinal carbon atoms on the aromatic ring,
R¹² and R¹³ are each independently a hydrogen atom or a C₁-C₂₀ hydrocarbyl group which may contain a heteroatom, R¹² and R¹³ may bond together to form a ring with the carbon atom to which they are attached,
R¹⁴ is a halogen atom, a hydroxy group, a cyano group, a nitro group, a C₁-C₂₀ hydrocarbyl group which may contain a heteroatom, a C₁-C₂₀ hydrocarbyloxy group which may contain a heteroatom, a C₂-C₂₀ hydrocarbyloxycarbonyl group which may contain a heteroatom, a C₁-C₂₀ hydrocarbylthio group which may contain a heteroatom, or —N(R^{14A})(R^{14B}), R^{14A} and R^{14B} are each independently a hydrogen atom or a C₁-C₆ hydrocarbyl group, and a plurality of R¹⁴ may be identical or different and the plurality of R¹⁴ may bond together to form a ring with the carbon atoms on the aromatic ring to which they are attached when b2 is 2 or more.

12. The chemically amplified resist composition of claim 11 wherein the base polymer comprises repeat units of at least one type selected from repeat units having the formula (b1) and repeat units having the formula (b2): 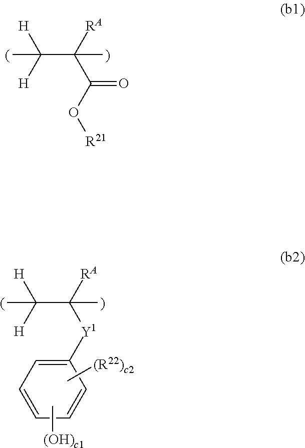
wherein R^{A} is each independently a hydrogen atom, a fluorine atom, a methyl group, or a trifluoromethyl group,
Y¹ is a single bond or *—C(═O)—O—, * designates a point of attachment to the carbon atom in the backbone,
R²¹ is a hydrogen atom or a C₁-C₂₀ group containing at least one structure selected from a hydroxy group other than a phenolic hydroxy group, a cyano group, a carbonyl group, a carboxy group, an ether bond, an ester bond, a sulfonate ester bond, a carbonate bond, a lactone ring, a sultone ring, and a carboxylic anhydride (—C(═O)—O—C(═O)—),
R²² is a halogen atom, a hydroxy group, a carboxy group, a nitro group, a cyano group, a C₁-C₂₀ hydrocarbyl group which may contain a heteroatom, a C₁-C₂₀ hydrocarbyloxy group which may contain a heteroatom, a C₂-C₂₀ hydrocarbylcarbonyl group which may contain a heteroatom, a C₂-C₂₀ hydrocarbylcarbonyloxy group which may contain a heteroatom, or a C₂-C₂₀ hydrocarbyloxycarbonyl group which may contain a heteroatom, a plurality of R²² may be identical or different when c2 is 2, 3, or 4,
c1 is 1, 2, 3, or 4, c2 is 0, 1, 2, 3, or 4, and provided that 1≤c1+c2≤5.

13. The chemically amplified resist composition of claim 11 wherein the base polymer comprises repeat units of at least one type selected from repeat units having the formula (c1), repeat units having the formula (c2), repeat units having the formula (c3), repeat units having the formula (c4), and repeat units having the formula (c5): 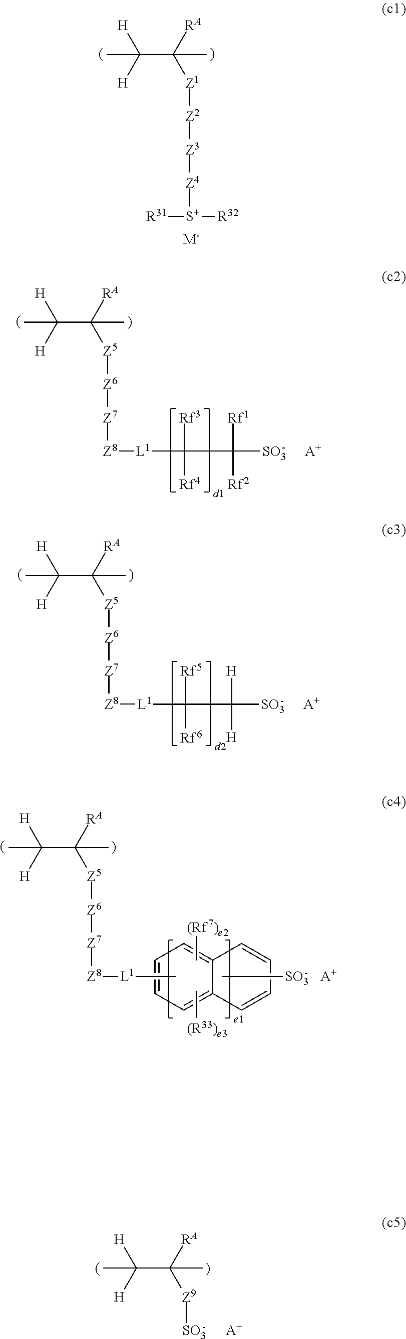
wherein d1 and d2 are each independently 0, 1, 2, or 3, e1 is 0 or 1, e2 is 0, 1, 2, 3, or 4, e3 is 0, 1, 2, 3, or 4, provided that 0≤e2+e3≤4 when e1 is 0 and 0≤e2+e3≤6 when e1 is 1,
R^{A} is each independently a hydrogen atom, a fluorine atom, a methyl group, or a trifluoromethyl group,
Z¹ is a single bond or an optionally substituted phenylene group,
Z² is a single bond, **—C(═O)—O—Z²¹—, **—C(═O)—NH—Z²¹—, or **—O—Z²¹—, Z²¹ is a C₁-C₆ aliphatic hydrocarbylene group, a phenylene group, or a divalent group obtained by combining the foregoing, which may contain a halogen atom, a carbonyl group, an ester bond, an ether bond, or a hydroxy group,
Z³ is a single bond, an ether bond, an ester bond, an amide bond, a sulfonate ester bond, a sulfonamide bond, a carbonate bond, or a carbamate bond,
Z⁴ is a single bond, or a C₁-C₆ aliphatic hydrocarbylene group, a phenylene group, or a divalent group obtained by combining the foregoing, which may contain a halogen atom, a carbonyl group, an ester bond, an ether bond, or a hydroxy group,
Z⁵ is each independently a single bond, an optionally substituted phenylene group, a naphthylene group, or *—C(═O)—O—Z⁵¹—, Z⁵¹ is a C₁-C₁₀ aliphatic hydrocarbylene group, a phenylene group, or a naphthylene group which may contain a halogen atom, a hydroxy group, an ether bond, an ester bond, or a lactone ring,
Z⁶ is a single bond, an ether bond, an ester bond, an amide bond, a sulfonate ester bond, a sulfonamide bond, a carbonate bond, or a carbamate bond,
Z⁷ is each independently a single bond, ***—Z⁷¹—C(═O)—O—, ***—C(═O)—NH—Z⁷¹—, or ***—O—Z⁷¹—, Z⁷¹ is a C₁-C₂₀ hydrocarbylene group which may contain a heteroatom,
Z⁸ is each independently a single bond, ****—Z⁸¹—C(═O)—O—, ****—C(═O)—NH—Z⁸¹—, or ****—O—Z⁸¹—, Z⁸¹ is a C₁-C₂₀ hydrocarbylene group which may contain a heteroatom,
Z⁹ is a single bond, a methylene group, an ethylene group, a phenylene group, a fluorinated phenylene group, a trifluoromethyl-substituted phenylene group, *—C(═O)—O—Z⁹¹—, *—C(═O)—N(H)—Z⁹¹—, or *—O—Z⁹¹—, Z⁹¹ is a C₁-C₆ aliphatic hydrocarbylene group, a phenylene group, a fluorinated phenylene group, or a trifluoromethyl-substituted phenylene group, which may contain a carbonyl group, an ester bond, an ether bond, or a hydroxy group,
* designates a point of attachment to the carbon atom in the backbone, ** designates a point of attachment to Z¹, *** designates a point of attachment to Z⁶, **** designates a point of attachment to Z⁷,
L¹ is a single bond, an ether bond, an ester bond, a carbonyl group, a sulfonate ester bond, a sulfonamide bond, a carbonate bond, or a carbamate bond,
Rf¹ and Rf² are each independently a fluorine atom or a C₁-C₆ fluorinated saturated hydrocarbyl group,
Rf³ and Rf⁴ are each independently a hydrogen atom, a fluorine atom, or a C₁-C₆ fluorinated saturated hydrocarbyl group,
Rf⁵ and Rf⁶ are each independently a hydrogen atom, a fluorine atom, or a C₁-C₆ fluorinated saturated hydrocarbyl group, excluding that all Rf⁵ and Rf⁶ are hydrogen atoms at the same time,
Rf⁷ is a fluorine atom, a C₁-C₆ fluorinated alkyl group, a C₁-C₆ fluorinated alkoxy group, or a C₁-C₆ fluorinated alkylthio group,
R³¹ and R³² are each independently a C₁-C₂₀ hydrocarbyl group which may contain a heteroatom, R³¹ and R³² may bond together to form a ring with the sulfur atom to which they are attached,
R³³ is a halogen atom other than a fluorine atom or a C₁-C₂₀ hydrocarbyl group which may contain a heteroatom, when e3 is 2, 3, or 4, a plurality of R³³ may be identical or different, and the plurality of R³³ may bond together to form a ring with the carbon atoms to which they are attached,
M⁻ is a non-nucleophilic counter ion, and
A⁺ is an onium cation.

14. The chemically amplified resist composition of claim 10, further comprising an organic solvent.

15. The chemically amplified resist composition of claim 10, further comprising an acid generator capable of generating an acid with pKa≤−2.0.

16. The chemically amplified resist composition of claim 10, further comprising a quencher other than the quencher.

17. The chemically amplified resist composition of claim 10, further comprising a surfactant.

18. A pattern forming process comprising the steps of applying the chemically amplified resist composition of claim 10 onto a substrate to form a resist film thereon, exposing the resist film to high-energy radiation, and developing the exposed resist film in a developer.

19. The pattern forming process of claim 18 wherein the high-energy radiation is KrF excimer laser radiation, ArF excimer laser radiation, EB, or EUV of wavelength 3 to 15 nm.
